SEQUENCE LISTING (165 sequences)

SEQ ID NO: 1 - DNA, 15 bp, Unknown
  Core region of the wildtype att site
gcttttttatactaa

SEQ ID NO: 2 - DNA, 21 bp, Unknown
  Reference sequence for att site seven base pair overlap region
caacttttttatacaaagttg

SEQ ID NO: 3 - DNA, 25 bp, Artificial Sequence
  attB1 site
agcctgcttttttgtacaaacttgt

SEQ ID NO: 4 - DNA, 233 bp, Artificial Sequence
  attP1 site
tacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttg

SEQ ID NO: 5 - DNA, 100 bp, Artificial Sequence
  attL1 site
caaataatgattttattttgactgatagtgacctgttcgttgcaacaaattgataagcaatgcttttttataatgccaactttgtacaaaaaagcaggct

SEQ ID NO: 6 - DNA, 125 bp, Artificial Sequence
  attR1 site
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 7 - DNA, 27 bp, Artificial Sequence
  attB0 site
agcctgcttttttatactaacttgagc

SEQ ID NO: 8 - DNA, 27 bp, Artificial Sequence
  attP0 site
gttcagcttttttatactaagttggca

SEQ ID NO: 9 - DNA, 27 bp, Artificial Sequence
  attL0 site
agcctgcttttttatactaagttggca

SEQ ID NO: 10 - DNA, 27 bp, Artificial Sequence
  attR0 site
gttcagcttttttatactaacttgagc

SEQ ID NO: 11 - DNA, 25 bp, Artificial Sequence
  attB1 site
agcctgcttttttgtacaaacttgt

SEQ ID NO: 12 - DNA, 27 bp, Artificial Sequence
  attP1 site
gttcagcttttttgtacaaagttggca

SEQ ID NO: 13 - DNA, 27 bp, Artificial Sequence
  attL1 site
agcctgcttttttgtacaaagttggca

SEQ ID NO: 14 - DNA, 25 bp, Artificial Sequence
  attR1 site
gttcagcttttttgtacaaacttgt

SEQ ID NO: 15 - DNA, 25 bp, Artificial Sequence
  attB2 site
acccagctttcttgtacaaagtggt

SEQ ID NO: 16 - DNA, 27 bp, Artificial Sequence
  attP2 site
gttcagctttcttgtacaaagttggca

SEQ ID NO: 17 - DNA, 27 bp, Artificial Sequence
  attL2 site
acccagctttcttgtacaaagttggca

SEQ ID NO: 18 - DNA, 25 bp, Artificial Sequence
  attR2 site
gttcagctttcttgtacaaagtggt

SEQ ID NO: 19 - DNA, 22 bp, Artificial Sequence
  attB5 site
caactttattatacaaagttgt

SEQ ID NO: 20 - DNA, 27 bp, Artificial Sequence
  attP5 site
gttcaactttattatacaaagttggca

SEQ ID NO: 21 - DNA, 24 bp, Artificial Sequence
  attL5 site
caactttattatacaaagttggca

SEQ ID NO: 22 - DNA, 25 bp, Artificial Sequence
  attR5 site
gttcaactttattatacaaagttgt

SEQ ID NO: 23 - DNA, 22 bp, Artificial Sequence
  attB11 site
caacttttctatacaaagttgt

SEQ ID NO: 24 - DNA, 27 bp, Artificial Sequence
  attP11 site
gttcaacttttctatacaaagttggca

SEQ ID NO: 25 - DNA, 24 bp, Artificial Sequence
  attL11 site
caacttttctatacaaagttggca

SEQ ID NO: 26 - DNA, 25 bp, Artificial Sequence
  attR11 site
gttcaacttttctatacaaagttgt

SEQ ID NO: 27 - DNA, 22 bp, Artificial Sequence
  attB17 site
caacttttgtatacaaagttgt

SEQ ID NO: 28 - DNA, 27 bp, Artificial Sequence
  attP17 site
gttcaacttttgtatacaaagttggca

SEQ ID NO: 29 - DNA, 24 bp, Artificial Sequence
  attL17 site
caacttttgtatacaaagttggca

SEQ ID NO: 30 - DNA, 25 bp, Artificial Sequence
  attR17 site
gttcaacttttgtatacaaagttgt

SEQ ID NO: 31 - DNA, 22 bp, Artificial Sequence
  attB19 site
caactttttcgtacaaagttgt

SEQ ID NO: 32 - DNA, 27 bp, Artificial Sequence
  attP19 site
gttcaactttttcgtacaaagttggca

SEQ ID NO: 33 - DNA, 24 bp, Artificial Sequence
  attL19 site
caactttttcgtacaaagttggca

SEQ ID NO: 34 - DNA, 25 bp, Artificial Sequence
  attR19 site
gttcaactttttcgtacaaagttgt

SEQ ID NO: 35 - DNA, 22 bp, Artificial Sequence
  attB20 site
caactttttggtacaaagttgt

SEQ ID NO: 36 - DNA, 27 bp, Artificial Sequence
  attP20 site
gttcaactttttggtacaaagttggca

SEQ ID NO: 37 - DNA, 24 bp, Artificial Sequence
  attL20 site
caactttttggtacaaagttggca

SEQ ID NO: 38 - DNA, 25 bp, Artificial Sequence
  attR20  site
gttcaactttttggtacaaagttgt

SEQ ID NO: 39 - DNA, 22 bp, Artificial Sequence
  attB21 site
caactttttaatacaaagttgt

SEQ ID NO: 40 - DNA, 27 bp, Artificial Sequence
  attP21 site
gttcaactttttaatacaaagttggca

SEQ ID NO: 41 - DNA, 24 bp, Artificial Sequence
  attL21 site
caactttttaatacaaagttggca

SEQ ID NO: 42 - DNA, 25 bp, Artificial Sequence
  attR21 site
gttcaactttttaatacaaagttgt

SEQ ID NO: 43 - DNA, 21 bp, Artificial Sequence
  att system core integrase binding site
caactttnnnnnnnaaagttg

SEQ ID NO: 44 - DNA, 21 bp, Artificial Sequence
  attB core integrase binding site
caactttnnnnnnnaaacaag

SEQ ID NO: 45 - DNA, 20 bp, Unknown
  T7 promoter or priming site
taatacgactcactataggg

SEQ ID NO: 46 - DNA, 21 bp, Unknown
  V5 reverse priming site
accgaggagagggttagggat

SEQ ID NO: 47 - DNA, 24 bp, Unknown
  pAd forward priming site
gactttgaccgtttacgtggagac

SEQ ID NO: 48 - DNA, 24 bp, Unknown
  pAd reverse priming site
ccttaagccacgcccacacatttc

SEQ ID NO: 49 - protein, 14 aa, SV5 paramyxovirus
GKPIPNPLLGLDST

SEQ ID NO: 50 - protein, 6 aa, Artificial Sequence
  6x His tag
HHHHHH

SEQ ID NO: 51 - DNA, 30 bp, Unknown
  pIB Neg For oligonucleotide
tgagtcaagggctgccgggctgcagcactg

SEQ ID NO: 52 - DNA, 32 bp, Unknown
  pIB Neg Rev oligonucleotide
cggaacaagggcatgaccaaaatcccttaacg

SEQ ID NO: 53 - DNA, 33 bp, Unknown
  gp64 For oligonucleotide
gactcaaagggcttgcttgtgtgttccttattg

SEQ ID NO: 54 - DNA, 34 bp, Unknown
  gp64s Rev oligonucleotide
gttccgaagggttgtgtcacgtaggccagataac

SEQ ID NO: 55 - DNA, 38 bp, Unknown
  gp64L Rev oligonucleotide
gttccgaagggaataatcgatttaagggtgtaatactc

SEQ ID NO: 56 - DNA, 33 bp, Unknown
  pe38 For oligonucleotide
gactcaaagggtttgcttattggcaggctctcc

SEQ ID NO: 57 - DNA, 31 bp, Unknown
  pe34s Rev oligonucleotide
gttccgaagggtatctgtcccccactcaggc

SEQ ID NO: 58 - DNA, 32 bp, Unknown
  pe38L Rev oligonucleotide
gttccgaagggtaaagttgatgcggcgacggc

SEQ ID NO: 59 - DNA, 27 bp, Unknown
  EcoRI sense primer
gaattccagctgagcgccggtcgctac

SEQ ID NO: 60 - DNA, 33 bp, Unknown
  BglII antisense primer
agatcttcattcattctcaccactttgtacaag

SEQ ID NO: 61 - DNA, 27 bp, Unknown
  V5/His 5 prime primer
agatctggggaagcctatccctaaccc

SEQ ID NO: 62 - DNA, 32 bp, Unknown
  V5/His 3 prime primer
agatcttcaatggtgatggtgatgatgaccgg

SEQ ID NO: 63 - DNA, 28 bp, Unknown
  Topo D1 oligonucleotide
aattgatcccttcaccgacatagtacag

SEQ ID NO: 64 - DNA, 12 bp, Unknown
  Topo D2 oligonucleotide
ggtgaagggatc

SEQ ID NO: 65 - DNA, 22 bp, Unknown
  Topo D6 oligonucletide
tcgagcccttgacatagtacag

SEQ ID NO: 66 - DNA, 21 bp, Unknown
  CMV forward primer
cgcaaatgggcggtaggcgtg

SEQ ID NO: 67 - DNA, 21 bp, Unknown
  V5 reverse primer
accgaggagagggttagggat

SEQ ID NO: 68 - DNA, 22 bp, Unknown
  UB forward primer
tcagtgttagactagtaaattg

SEQ ID NO: 69 - DNA, 15 bp, Unknown
  DNA sequence of the N-terminus of a theoretical protein
atgggatctgataaa

SEQ ID NO: 70 - DNA, 19 bp, Unknown
  Proposed PCR primer for theoretical protein
caccatgggatctgataaa

SEQ ID NO: 71 - DNA, 27 bp, Unknown
  DNA sequence of the C terminus of a theoretical protein
aagtcggagcactcgacgacggtgtag

SEQ ID NO: 72 - DNA, 17 bp, Unknown
  Proposed reverse PCR primer for C terminus of the theoretical protein
aaacaccgtcgtcgagt

SEQ ID NO: 73 - DNA, 33 bp, Unknown
  Sequence for the C-terminus of a theoretical protein
gcggttaagtcggagcactcgacgactgcatag

SEQ ID NO: 74 - DNA, 24 bp, Unknown
  Reverse primer for fusing the ORF of a theoretical protein in frame with the C terminal tag in pLenti6 V5 D TOPO
tgcagtcgtcgagtgctccgactt

SEQ ID NO: 75 - DNA, 27 bp, Unknown
  Reverse primer for avoiding fusing the ORF of a theoretical protein in frame with the C terminal tag in pLenti6 V5 D TOPO
ctatgcagtcgtcgagtgctccgactt

SEQ ID NO: 76 - DNA, 39 bp, Unknown
  tetO2 forward primer
gactcgagtctccctatcagtgatagagatctcgaggtc

SEQ ID NO: 77 - DNA, 39 bp, Unknown
  tetO2 reverse primer
gacctcgagatctctatcactgatagggagactcgagtc

SEQ ID NO: 78 - DNA, 24 bp, Unknown
  Forward PCR primer
caccatggagaaaaaaatcactgg

SEQ ID NO: 79 - DNA, 19 bp, Unknown
  Reverse PCR primer
ctgctacgccccgccctgc

SEQ ID NO: 80 - DNA, 37 bp, Unknown
  Forward PCR primer
caccgaattctctagagatgtctgtgaaaagaaacat

SEQ ID NO: 81 - DNA, 37 bp, Unknown
  Reverse PCR primer
atataagcttactagtccggatttcctctacccgaga

SEQ ID NO: 82 - DNA, 22 bp, Unknown
  GFP reverse priming site
gggtaagctttccgtatgtagc

SEQ ID NO: 83 - DNA, 36686 bp, Artificial Sequence
  pAd CMV V5 DEST
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 84 - DNA, 34226 bp, Artificial Sequence
  pAd GW TO tRNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctttaaaggaaccaattcagtcgactctagaggatcgaaaccatcctctgctatatggccgcatatattttacttgaagactaggaccctacagaaaaggggttttaaagtaggcgtgctaaacgtcagcggacctgacccgtgtaagaatccacaaggtatcctggtggaaatgcgcatttgtaggcttcaatatctgtaatcctactaattaggtgtggagagctttcagccagtttcgtaggtttggagaccatttaggggttggcgtgtggccccctcgtaaagtctttcgtacttcctacatcagacaagtcttgcaatttgcaatatctcttttagccaatatctaaatctttaaaattttgattttgttttttacccaggatgagagacattccagagttgttaccttgtcaaaataaacaaatttaaagatgtctgtgaaaagaaacatatattcctcatgggaatatatccaggttgttgaaggaggtacgacctcgagatctctatcactgatagggagactcgagtgtagtcgtggccgagtggttaaggcgatggactctaaatccattggggtctccccgcgcaggttcgaatcctgccgactacggcgtgctttttttactctcgggtagaggaaatccggtgcactacctgtgcaatcacacagaataacatggagtagtactttttattttcctgttattatctttctccataaaagtggaaccagataattttagttcttttgtgtaacaagactagagattttttgaagtgttacattggaaagcacttgaaaacacaagtaatttctgacactgctataaaaatgatggaaaaacgctcaagttgttttgcctttcagtcttcttgaaatgctgtctccctatctgaaatccagctcacgtctgacttccaaaaccgtgcttgcctttaacttatggaataaatatctcaaacagatccccgggcgagctcgaattcgcggccgcactcgagatatctagacccagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 85 - DNA, 34226 bp, Artificial Sequence; identical to SEQ ID NO: 84
  pAdenoTAG tRNA

SEQ ID NO: 86 - DNA, 954 bp, Unknown
  Sau3A fragment used to construct vectors comprising suppressor tRNA sequences
ctagaggatcgaaaccatcctctgctatatggccgcatatattttacttgaagactaggaccctacagaaaaggggttttaaagtaggcgtgctaaacgtcagcggacctgacccgtgtaagaatccacaaggtatcctggtggaaatgcgcatttgtaggcttcaatatctgtaatcctactaattaggtgtggagagctttcagccagtttcgtaggtttggagaccatttaggggttggcgtgtggccccctcgtaaagtctttcgtacttcctacatcagacaagtcttgcaatttgcaatatctcttttagccaatatctaaatctttaaaattttgattttgttttttaaccaggatgagagacattccagagttgttaccttgtcaaaataaacaaatttaaagatgtctgtgaaaagaaacatatattcctcatgggaatatatccaggttgttgaaggaggtacactcgagtctccctatcagtgatagagatctcgaggtcgtagtcgtggccgagtggttaaggcgatggactctaaatccattggggtctccccgcgcaggttcgaatcctgccgactacggcgtgctttttttactctcgggtagaggaaatccggtgcactacctgtgcaatcacacagaataacatggagtagtactttttattttcctgttattatctttctccataaaagtggaaccagataattttagttcttttgtgtaacaagactagagattttttgaagtgttacattggaaagcacttgaaaacacaagtaatttctgacactgctataaaaatgatggaaaaacgctcaagttgttttgcctttcagtcttcttgaaatgctgtctccctatctgaaatccagctcacgtctgacttccaaaaccgtgcttgcctttaacttatggaataaatatctcaaacagatcccc

SEQ ID NO: 87 - DNA, 34864 bp, Artificial Sequence
  pAd/PL-DEST
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 88 - DNA, 37567 bp, Artificial Sequence
  pAd/CMV/V5-GW/lacZ.PL-DEST
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctccgcggccgcccccttcaccatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggagttccagctgagcgccggtcgctaccattaccagttggtctggtgtcaaaaaactaagggtgggcgcgccgacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 89 - DNA, 5038 bp, Artificial Sequence
  pIB/V5-His-DEST
catgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcacgtgaccggacacgaggcgcccgtcccgcttatcgcgcctataaatacagcccgcaacgatctggtaaacacagttgaacagcatctgttcgaatttaaagcttgatatcgaattcctgcagcccagcgctggatcctcgatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgacccgggtctagagggcccgcggttcgaaggtaagcctatccctaaccctctcctcggtctcgattctacgcgtaccggtcatcatcaccatcaccattgagtttatctgactaaatcttagtttgtattgtcatgttttaatacaatatgttatgtttaaatatgtttttaataaattttataaaataatttcaacttttattgtaacaacattgtccatttacacactcctttcaagcgcgtgggatcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcccttgttccgaagggttgtgtcacgtaggccagataacggtcgggtatataagatgcctcaatgctactagtaaatcagtcacaccaaggcttcaataaggaacacacaagcaagccctttgagtcaagggctgccgggctgcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccggcgcagctctctctagcgacggccgcatcttcactggtgtcaatgtatatcattttactgggggaccttgcgcagaactcgtggtgctgggcactgctgctgctgcggcagctggcaacctgacttgtatcgtcgcgatcggaaatgagaacaggggcatcttgagcccctgcggacggtgccgacaggttcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtcccgggagatctgcatgtctactaaactcacaaattagagcttcaatttaattatatcagttattacccattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatct

SEQ ID NO: 90 - DNA, 5693 bp, Artificial Sequence
  V5-His DEST cassette
ataagtattttactgttttcgtaacagttttgtaataaaaaaacctataaatattccggattattcataccgtcccaccatcgggcgcggatccccgggtaccgatatcacaagtttgtacaaaaaagctgaacgagaaacgtaaaatgatataaatatcaatatattaaattagattttgcataaaaaacagactacataatactgtaaaacacaacatatccagtcactatggcggccgctccctaacccacggggcccgtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacatgcatgggacggaggcgtttggccaagacgtccaaagcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgcccccgaaagctgtccccaatcctcccgccacgacccgccgccctgcagataccgcaccgtattggcaagcagcccataaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgttgccggcgagggcgcaacgccgtacgtcggttgttatggccgcgagaacgcgcagcctggtcgaacgcagacgcgtgttgatggcaggggtacgaagccatagatcccgttatcaattacttatactatccggcgcgcaagcgagcgtgtgcgccggagcacaattgatactgatttacgagttgggcaaacgggctttatatagcctgtcccctccacagccctagtgccgtgcgcaaagtgcctacgtgaccaggctctcctacgcatatacaatcttatctctatagataaggtttccatatataaagcctctcgatggctgaacgtgcacagtatcgtgttgatttctgagtgctaactaacagttacaatgaaccgtttttttcgagagaataacatttttgacgcgccaaggaccgggggcaagggtcgtgccaaatctttgccagcgcctgccgccaactcgccgccgtcgcctgttcgtccgccgccaaaatctaacatcaaaccacctacgcgcatctctccgcctaaacagcctatgtgcacctctccggccaagccgttggagcacagcagcattgtaagtaaaaaaccagtcgtcaacagaaaagatggatattttgtgccgcccgagtttgggaacaagtttgaaggtttgcccgcgtacagcgacaaactggatttcaaacaagagcgcgatctacgtacctgcaggcccgggctcaacccaacacaatatattatagttaaataagaattattatcaaatcatttgtatattaattaaaatactatactgtaaattacattttatttacaattcactctagaatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataatgactgcaggtcgaccatagtgactggatatgttgtgttttacagtattatgtagtctgttttttatgcaaaatctaatttaatatattgatatttatatcattttacgtttctcgttcagctttcttgtacaaagtggtgagaatgaatgaagatctggggaagcctatccctaaccctctcctcggtctcgattctacgcgtaccggtcatcatcaccatcaccattga

SEQ ID NO: 91 - protein, 1024 aa, Artificial Sequence
  V5-His DEST cassette
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 92 - protein, 23 aa, Artificial Sequence
  V5-His DEST cassette
GKPIPNPLLGLDSTRTGHHHHHH

SEQ ID NO: 93 - protein, 376 aa, Herpesvirus sp.
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGAAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 94 - DNA, 5763 bp, Artificial Sequence
  Mel/V5-His DEST cassette
ataagtattttactgttttcgtaacagttttgtaataaaaaaacctataaatattccggattattcataccgtcccaccatcgggcgcggatcctataaatatgaaattcttagtcaacgttgcccttgtttttatggtcgtatacatttcttacatctatgcggcatggtcgaatcaaacaagtttgtacaaaaaagctgaacgagaaacgtaaaatgatataaatatcaatatattaaattagattttgcataaaaaacagactacataatactgtaaaacacaacatatccagtcactatggcggccgctccctaacccacggggcccgtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacatgcatgggacggaggcgtttggccaagacgtccaaagcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgcccccgaaagctgtccccaatcctcccgccacgacccgccgccctgcagataccgcaccgtattggcaagcagcccataaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgttgccggcgagggcgcaacgccgtacgtcggttgttatggccgcgagaacgcgcagcctggtcgaacgcagacgcgtgttgatggcaggggtacgaagccatagatcccgttatcaattacttatactatccggcgcgcaagcgagcgtgtgcgccggagcacaattgatactgatttacgagttgggcaaacgggctttatatagcctgtcccctccacagccctagtgccgtgcgcaaagtgcctacgtgaccaggctctcctacgcatatacaatcttatctctatagataaggtttccatatataaagcctctcgatggctgaacgtgcacagtatcgtgttgatttctgagtgctaactaacagttacaatgaaccgtttttttcgagagaataacatttttgacgcgccaaggaccgggggcaagggtcgtgccaaatctttgccagcgcctgccgccaactcgccgccgtcgcctgttcgtccgccgccaaaatctaacatcaaaccacctacgcgcatctctccgcctaaacagcctatgtgcacctctccggccaagccgttggagcacagcagcattgtaagtaaaaaaccagtcgtcaacagaaaagatggatattttgtgccgcccgagtttgggaacaagtttgaaggtttgcccgcgtacagcgacaaactggatttcaaacaagagcgcgatctacgtacctgcaggcccgggctcaacccaacacaatatattatagttaaataagaattattatcaaatcatttgtatattaattaaaatactatactgtaaattacattttatttacaattcactctagaatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataatgactgcaggtcgaccatagtgactggatatgttgtgttttacagtattatgtagtctgttttttatgcaaaatctaatttaatatattgatatttatatcattttacgtttctcgttcagctttcttgtacaaagtggtgagaatgaatgaagatctggggaagcctatccctaaccctctcctcggtctcgattctacgcgtaccggtcatcatcaccatcaccattga

SEQ ID NO: 95 - protein, 21 aa, Artificial Sequence
  Mel/V5-His DEST cassette
MKFLVNVALVFMVVYISYIYA

SEQ ID NO: 96 - protein, 1024 aa, Artificial Sequence; identical to SEQ ID NO: 91
  Mel/V5-His DEST cassette

SEQ ID NO: 97 - protein, 23 aa, Artificial Sequence
  Mel/V5-His DEST cassette
GKPIPNPLLGLDSTRTGHHHHHH

SEQ ID NO: 98 - DNA, 1021 bp, Unknown
  AcMNPV ORF 25  promoter sequence
ggtgtcttcattagtatgccaatcacgtacgcaacagtcgcaaaagaaacacacagtttcgtctccgcgacccgtgtaaaaaagtcccgcttccgcaatgtttgtaatcatgtcacgcaatgcggcaggccaaaagttaacaaacgtatccatacgcgactgtaaattggacatgcatctgtacacacacttgggtttgccttctttcactagtacagcgttgatggtaatgttgtcgccaaacgattcacgctcggcgatcttgttagcatacgcgcaatacggcgacaaggttacgtgtgcatattcaatacactcgtcttcggaccaattttttatttctgcttcgcaatactcgcacacaacgtgatcgtcaacttgattgtatttaaacccgttaacgatcaagctgttaataaacgccgtgttttcaatgggataattttcaaacgaactatgtctttctattaacatgtcgaatacgtgttcggcggtgttgtcgcgaaagttgtcacacacgctgataaaataaaacgggggcgtgtcctcgttcattttagctcgttaaagttacggtcaaaatgagcacgtttgcgtcgttttggtttagcgacacgtttatatggcccagtttggtttttgtttcggcgttaatgacgtgcactgtggacaaatcgtgttctaaaactacaaactcgtactcgaaaatgtttgatatgtagttggttagccgatctatcttaaaattaaacttttgcaactcgctgatagagcacacgtccacatacttgtcgataaacccgttgctcaaccgcttcaaaacggtgtaattttgtagcttgaaaggggcgcatttggaatgactaaaaggaatatttttcaataaatcgtcagtagtgtacgcaaacgcgttgtctacgcacatgctggcaacagagtcgtccatatttattatatatcttatattctgtgaaacacttcaattagacttgaaccacagcagacagcgcacgtcggtagc

SEQ ID NO: 99 - DNA, 1033 bp, Unknown
  AcMNPV lef 3 promoter
ccgagaagaaggcggtttgtataaaacccatttttcgaaatggttaacaaacttgtttagcatttggatcgtttcgtgttcaaacgcgtcgaaaacttttaaaacgcaattgccgccgggacgcaggcaaattaaaattagctgcgtctcgcacatgatcaaatcaaagttgagacgttcttgttcgttttcgcgtccattaacgtcaaccgagccatctgccaacaccagatcgcacgcgttgccacacttgatgctaatctcaaatacaacatttttatcaaacacgtcgcctgacttgtcgggccccgtaatggttgtgaaatttttgcgtttgcgcactgtcggtttgtacacgcacaccgagttgtttgtcaacgtgacgccatacgctttgcaaagcgggttcaacgacatggtatagttggcaaactcgcccggtccgccgcacaaatccaaaaacgtgtcaacgtgtcggcaaacgtgaaactttttgtcgatctctgatagttttcgccaacatctaggtctgcgcgttgggcgtttgtcaaataattttgagcgagcgcaaaccaccgacttgctgctgaacgtgttcaaaccatctttgagtttatttaatttttgctgcaacatttttactcttcgtgtcggtcgcaatgtttgtgtcgaaaaagacggccaacacgctcagcaaaactatacaaataaagaacaaaaatacgtacgcaatattaacattgaccgtttgatcgttaaatcggacgggtctgttcagagccgctcttattctctcgttgtacattgttaaagtttttgtttttaaattgtacacaatcggcgtgttgtagtcgaaattttcaaaatcggctttttgaaacattgttctgaacgtgttgtcgagcggcgtgttgctggccacgtttataatcaactccctccacgctaacgaacggtgctctggcgacacttcgatttcgtcgccattcagtatttgccatcggatagattcccacatatcgacaacagcaat

SEQ ID NO: 100 - DNA, 1053 bp, Unknown
  AcMNPV TLP promoter
tgctagcccaattggccactgttgtacgaaatatcgtcgtcaacgtgtttgaatacatgttggcccgtaccgttgggtaaatctatgcatctggagtcgccggaacactcgtactggttgtcagagtttctgatccggttgatgcacgttatcagttgtgactcgttattattcaaacatttgaaatattgcgtgtcgccgatatcggccgttatgtacgtgtgtccggcgccgttaaacgcgcacggatgcgcttccacgcacgacattaagttgcgatcaaatattttattcgcggggcattcgcccaccacgtggcgcccatttacgcactgcataaactggttgacgagcaaattggagggaaagtatgatagtatatagccgtctggcctgttttcacacaattcgttaactttacactggccggtttccgcgtcaaacgtgtaattatctggacattcttcgactgcgtgcgctccgtttgcaaaacacctaagatagaacgtgggatgatacaagtgcgcgttggtagaataatctttgtccaagtgttggttcaacaccaacgtgtccagcaaacgctcgtccatgggataaagaccggcagacttgttgtcgcacggcggcacgggaacacattttagttgtgcgtaatcaaagttaaaatatgcggggcatttcatggtcacgtcggccttgtcgccgctcaaaataaactcgttgggattttcatcatttgctctaacgcgatcgtgtacgattcgatcaacaggttgaaatttttgatttaagaaatcaaaaatttcaatccggtcatcatgcacgctttcgtgataggtggaaaggtcgacggtgttgaaccacgttacaatataagtgttttgcataatatccgacacgtagcctattacgtcgggtgtgggttcgtctgcgttggtgcgcttcacatattcagtcatcacttggagccgcttggtgaaagtcgtttcgtcaaattcaaaataaattgccaaatacattaaagtaaacgctattataagaaaaaagctt

SEQ ID NO: 101 - DNA, 507 bp, Unknown
  AcMNPV hr5 sequence
gttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaacaaatgactcatcaatcgatcgtgcgtacacgtagaattctactcgtaaagcgagtttatgagccgtgtgcaaaacatgacatcatctcgatttgaaaaacaaatgacatcatccactgatcgtgcgttacaagtagaattctactcgtaaagccagttcggttatgagccgtgtgcaaaacatgacatcagcttatgactcgtacttgattgtgttttacgcgtagaattctactcgtaaagc

SEQ ID NO: 102 - DNA, 507 bp, Unknown; identical to SEQ ID NO: 101
  AcMNPV IE-1 promoter

SEQ ID NO: 103 - DNA, 1746 bp, Unknown
  AcMPNV IE-1 coding sequence
atgacgcaaattaattttaacgcgtcgtacaccagcgcttcgacgccgtcccgagcgtcgttcgacaacagctattcagagttttgtgataaacaacccaacgactatttaagttattataaccatcccaccccggatggagccgacacggtgatatctgacagcgagactgcggcagcttcaaactttttggcaagcgtcaactcgttaactgataatgatttagtggaatgtttgctcaagaccactgataatctcgaagaagcagttagttctgcttattattcggaatcccttgagcagcctgttgtggagcaaccatcgcccagttctgcttatcatgcggaatcttttgagcattctgctggtgtgaaccaaccatcggcaactggaactaaacggaagctggacgaatacttggacaattcacaaggtgtggtgggccagtttaacaaaattaaattgaggcctaaatacaagaaaagcacaattcaaagctgtgcaacccttgaacagacaattaatcacaacacgaacatttgcacggtcgcttcaactcaagaaattacgcattattttactaatgattttgcgccgtatttaatgcgtttcgacgacaacgactacaattccaacaggttctccgaccatatgtccgaaactggttattacatgtttgtggttaaaaaaagtgaagtgaagccgtttgaaattatatttgccaagtacgtgagcaatgtggtttacgaatatacaaacaattattacatggtagataatcgcgtgtttgtggtaacttttgataaaattaggtttatgatttcgtacaatttggttaaagaaaccggcatagaaattcctcattctcaagatgtgtgcaacgacgagacggctgcacaaaattgtaaaaaatgccatttcgtcgatgtgcaccacacgtttaaagctgctctgacttcatattttaatttagatatgtattacgcgcaaaccacatttgtgactttgttacaatcgttgggcgaaagaaaatgtgggtttcttttgagcaagttgtacgaaatgtatcaagataaaaatttatttactttgcctattatgcttagtcgtaaagagagtaatgaaattgagactgcatctaataatttctttgtatcgccgtatgtgagtcaaatattaaagtattcggaaagtgtgcagtttcccgacaatcccccaaacaaatatgtggtggacaatttaaatttaattgttaacaaaaaaagtacgctcacgtacaaatacagcagcgtcgctaatcttttgtttaataattataaatatcatgacaatattgcgagtaataataacgcagaaaatttaaaaaaggttaagaaggaggacggcagcatgcacattgtcgaacagtatttgactcagaatgtagataatgtaaagggtcacaattttatagtattgtctttcaaaaacgaggagcgattgactatagctaagaaaaacaaagagttttattggatttctggcgaaattaaagatgtagacgttagtcaagtaattcaaaaatataatagatttaagcatcacatgtttgtaatcggtaaagtgaaccgaagagagagcactacattgcacaataatttgttaaaattgttagctttaatattacagggtctggttccgttgtccgacgctataacgtttgcggaacaaaaactaaattgtaaatataaaaaattcgaatttaat

SEQ ID NO: 104 - protein, 582 aa, Unknown
  AcMNPV IE-1 protein sequence
MTQINFNASYTSASTPSRASFDNSYSEFCDKQPNDYLSYYNHPTPDGADTVISDSETAAASNFLASVNSLTDNDLVECLLKTTDNLEEAVSSAYYSESLEQPVVEQPSPSSAYHAESFEHSAGVNQPSATGTKRKLDEYLDNSQGVVGQFNKIKLRPKYKKSTIQSCATLEQTINHNTNICTVASTQEITHYFTNDFAPYLMRFDDNDYNSNRFSDHMSETGYYMFVVKKSEVKPFEIIFAKYVSNVVYEYTNNYYMVDNRVFVVTFDKIRFMISYNLVKETGIEIPHSQDVCNDETAAQNCKKCHFVDVHHTFKAALTSYFNLDMYYAQTTFVTLLQSLGERKCGFLLSKLYEMYQDKNLFTLPIMLSRKESNEIETASNNFFVSPYVSQILKYSESVQFPDNPPNKYVVDNLNLIVNKKSTLTYKYSSVANLLFNNYKYHDNIASNNNAENLKKVKKEDGSMHIVEQYLTQNVDNVKGHNFIVLSFKNEERLTIAKKNKEFYWISGEIKDVDVSQVIQKYNRFKHHMFVIGKVNRRESTTLHNNLLKLLALILQGLVPLSDAITFAEQKLNCKYKKFEFN

SEQ ID NO: 105 - DNA, 8688 bp, Artificial Sequence
  pLenti6/V5-DEST
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 106 - DNA, 6964 bp, Artificial Sequence
  pLenti6/V5-D-TOPO
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattgatcccttcaccaagggctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 107 - DNA, 8634 bp, Artificial Sequence
  pLenti4/V5-DEST
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 108 - DNA, 9320 bp, Artificial Sequence
  pLenti6/UbC/V5-DEST
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccactagtccagtgtggtggaattctgcagatatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 109 - DNA, 8889 bp, Artificial Sequence
  pLP1
ttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgggatcccctgagggggcccccatgggctagaggatccggcctcggcctctgcataaataaaaaaaattagtcagccatgagc

SEQ ID NO: 110 - DNA, 4180 bp, Artificial Sequence
  pLP2
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgatgtacgggccagatatacgcgtatctgaggggactagggtgtgtttaggcgaaaagcggggcttcggttgtacgcggttaggagtcccctcaggatatagtagtttcgcttttgcatagggaggggga

SEQ ID NO: 111 - DNA, 5821 bp, Artificial Sequence
  pLP/VSVG
ttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgggatcccctgagggggcccccatgggctagaggatccggcctcggcctctgcataaataaaaaaaattagtcagccatgagc

SEQ ID NO: 112 - DNA, 7341 bp, Artificial Sequence
  pcDNA6.2/V5-DEST
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 113 - DNA, 7995 bp, Artificial Sequence
  pcDNA6.2/GFP-DEST
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggttgatctagagggccccgcggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgtcacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaatagtaatgagtccacgtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 114 - protein, 265 aa, Unknown
  Amino acid sequence of a polypeptide having beta-lactamase activity
MGHPETLVKVKDAEDQLGARVGYIELDLNSGKILESFRPEERFPMMSTFKVLLCGAVLSRDDAGQEQLGRRIHYSQNDLVEYSPVTEKHLTDGMTVRELCSAAITMSDNTAANLLLTTIGGPKELTAFLHNMGDHVTRLDHWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 115 - DNA, 8599 bp, Artificial Sequence
  pLenti4TO/V5-DEST
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgactagtccagtgtggtggaattctgcagatatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 116 - DNA, 8355 bp, Artificial Sequence
  pLenti6/TR
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatagcttggtacccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattctagagggcccgcggttcgaacaaaaactcatctcagaagaggatctgaatatgcataccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 117 - DNA, 6975 bp, Artificial Sequence
  pLenti6/V5
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 118 - DNA, 7428 bp, Artificial Sequence
  pLenti3/V5-TREx
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagaggatccctaccggtgatatcctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggcctcaattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagtttaaactggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 119 - DNA, 1474 bp, Unknown
  Nucleic acid fragment containing the tetracycline repressor coding sequence
agcttggtacccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattctagagggcccgcggttcgaacaaaaactcatctcagaagaggatctgaatatgcata

SEQ ID NO: 120 - DNA, 7056 bp, Artificial Sequence
  pRRL6/V5 also referred to as pLenti6/V5
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatccagcacagtggcggccgctcgagtctagagggcccgcggttcgaaggtaagcctatccctaaccctctcctcggtctcgattctacgcgtaccggttagtaatgagtttggcctgctgccggctctgcggcctcttccgcgtcttcgccttcgccctcagacgagtcggatctccctttgggccgcctccccgcctggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 121 - DNA, 172 bp, Artificial Sequence
  Recombination region of pAd/CMV/V5-DEST
ttgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctn

SEQ ID NO: 122 - DNA, 135 bp, Artificial Sequence
  Recombination region of pAd/CMV/V5-DEST
nacccagctttcttgtacaaagtggttgatctagagggcccgcggttcgaaggtaagcctatccctaaccctctcctcggtctcgattctacgcgtaccggttagtaatgagtttaaacgggggaggctaactga

SEQ ID NO: 123 - protein, 33 aa, Artificial Sequence
  Recombination region of pAd/CMV/V5-DEST
PAFLYKVVDLEGPRFEGKPIPNPLLGLDSTRTG

SEQ ID NO: 124 - DNA, 197 bp, Artificial Sequence
  Recombination region of pAd/PL-DEST
tatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctn

SEQ ID NO: 125 - DNA, 90 bp, Artificial Sequence
  Recombination region of pAd/PL-DEST
nacccagctttcttgtacaaagtggtgatcgattcgacagatcactgaaatgtgtgggcgtggcttaagggtgggaaagaatatataagg

SEQ ID NO: 126 - DNA, 560 bp, Unknown
  OpIE2 promoter sequence
ggatcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaattta

SEQ ID NO: 127 - DNA, 147 bp, Artificial Sequence
  Recombination region of pIB/V5-His-DEST
cttatcgcgcctataaatacagcccgcaacgatctggtaaacacagttgaacagcatctgttcgaatttaaagcttgatatcgaattcctgcagcccagcgctggatcctcgatcacaagtttgtacaaaaaagcaggctnnnnnnn

SEQ ID NO: 128 - DNA, 184 bp, Artificial Sequence
  Recombination region of pIB/V5-His-DEST
nacccaccagctttcttgtacaaagtggtgatcgacccgggtctagagggcccgcggttcgaaggtaagcctatccctaaccctctcctcggtctcgattctacgcgtaccggtcatcatcaccatcaccattgagtttatctgactaaatcttagttgtattgtcatgttttaatacaatatg

SEQ ID NO: 129 - protein, 43 aa, Artificial Sequence
  Recombination region of pIB/V5-His-DEST
PPAFLYKVVIDPGLEGPRFEGKPIPNPLLGLDSTRTGHHHHHH

SEQ ID NO: 130 - DNA, 215 bp, Artificial Sequence
  Recombination region of pLenti6/V5-DEST
tcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaacaagtttgtacaaaaaagcaggctn

SEQ ID NO: 131 - DNA, 142 bp, Artificial Sequence
  Recombination region of pLenti6/V5-DEST
nacccagctttcttgtacaaagtggttgatatccagcacagtggcggccgctcgagtctagagggcccgcggttcgaaggtaagcctatccctaaccctctcctcggtctcgattctacgcgtaccggttagtaatgagttt

SEQ ID NO: 132 - protein, 42 aa, Artificial Sequence
  Recombination region of pLenti6/V5-DEST
PAFLYKVVDIQHSGGRSSLEGPRFEGKPIPNPLLGLDSTRTG

SEQ ID NO: 133 - DNA, 217 bp, Artificial Sequence
  Recombination region of the expression clone resulting from pLenti6/UbC/V5-DEST x entry clone
ttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccactagtccagtgtggtggaattctgcagatatcaacaagtttgtacaaaaaagcaggctn

SEQ ID NO: 134 - DNA, 142 bp, Artificial Sequence; identical to SEQ ID NO: 131
  Recombination region of the expression clone resulting from pLenti6/UbC/V5-DEST x entry clone

SEQ ID NO: 135 - protein, 42 aa, Artificial Sequence; identical to SEQ ID NO: 132
  Recombination region of the expression clone resulting from pLenti6/UbC/V5-DEST x entry clone

SEQ ID NO: 136 - DNA, 1226 bp, Unknown
  Sequence of the UbC promoter
cggatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgg

SEQ ID NO: 137 - DNA, 32 bp, Unknown
  Directional cloning product of Figure 47
cccttcaccatgnnnnnnnnnnnnnnnaaggg

SEQ ID NO: 138 - DNA, 192 bp, Artificial Sequence
  Cloning region of pLenti6/V5-D-Topo
tcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattgatcccttcaccatg

SEQ ID NO: 139 - DNA, 101 bp, Artificial Sequence
  Cloning region of pLenti6/V5-D-Topo
aagggctcgagtctagagggcccgcggttcgaaggtaagcctatccctaaccctctcctcggtctcgattctacgcgtaccggttagtaatgagtttggaa

SEQ ID NO: 140 - protein, 28 aa, Artificial Sequence
  Cloning region of pLenti6/V5-D-Topo
KGSSLEGPRFEGKPIPNPLLGLDSTRTG

SEQ ID NO: 141 - DNA, 166 bp, Artificial Sequence
  Recombination region of pcDNA6.2/V5-DEST
caaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctn

SEQ ID NO: 142 - DNA, 144 bp, Artificial Sequence
  Recombination region of pcDNA6.2/V5-DEST
tagnacccagctttcttgtacaaagtggttgatctagagggcccgcggttcgaaggtaagcctatccctaaccctctcctcggtctcgattctacgcgtaccggttagtaatgagtttaaacgggggaggctaactgaaacacg

SEQ ID NO: 143 - protein, 33 aa, Artificial Sequence; identical to SEQ ID NO: 123
  Recombination region of pcDNA6.2/V5-DEST

SEQ ID NO: 144 - DNA, 166 bp, Artificial Sequence; identical to SEQ ID NO: 141
  Recombination region of pcDNA6.2/GFP-DEST

SEQ ID NO: 145 - DNA, 213 bp, Artificial Sequence
  Recombination region of pcDNA6.2/GFP-DEST
tagnacccagctttcttgtacaaagtggttgatctagagggccccgcggctagcaaaggagaagaacttttcactggaggtgtcccaattcttgttgaattagatggtgatgttaatgggcacaaattttctgtcagtggagagggtgaaggtgatgctacatacggaaagcttacccttaaatttatttgcactactggaaaactacctgtt

SEQ ID NO: 146 - protein, 69 aa, Artificial Sequence
  Recombination region of pcDNA6.2/GFP-DEST
PAFLYKVVDLEGPAASKGEELFTGGVPILVELDGDVNGHKFSVSGEGEGDATYGKLTLKFICTTGKLPV

SEQ ID NO: 147 - DNA, 307 bp, Artificial
  Recombination region of pAd/CMV/V5 DEST
ttgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctnnacccagctttcttgtacaaagtggttgatctagagggcccgcggttcgaaggtaagcctatccctaaccctctcctcggtctcgattctacgcgtaccggttagtaatgagtttaaacgggggaggctaactga

SEQ ID NO: 148 - DNA, 287 bp, Artificial
  Recombination region of pAd/PL DEST
tatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctnnacccagctttcttgtacaaagtggtgatcgattcgacagatcactgaaatgtgtgggcgtggcttaagggtgggaaagaatatataagg

SEQ ID NO: 149 - DNA, 325 bp, Artificial
  Recombination region of pIB/V5 His DEST
cttatcgcgcctataaatacagcccgcaacgatctggtaaacacagttgaacagcatctgttcgaatttaaagcttgatatcgaattcctgcagcccagcgctggatcctcgatcacaagtttgtacaaaaaagcaggctnnacccaccagctttcttgtacaaagtggtgatcgacccgggtctagagggcccgcggttcgaaggtaagcctatccctaaccctctcctcggtctcgattctacgcgtaccggtcatcatcaccatcaccattgagtttatctgactaaatcttagttgtattgtcatgttttaatacaatatg

SEQ ID NO: 150 - DNA, 357 bp, Artificial
  Recombination region of pLenti6/V5 DEST
tcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaacaagtttgtacaaaaaagcaggctnnacccagctttcttgtacaaagtggttgatatccagcacagtggcggccgctcgagtctagagggcccgcggttcgaaggtaagcctatccctaaccctctcctcggtctcgattctacgcgtaccggttagtaatgagttt

SEQ ID NO: 151 - DNA, 359 bp, Artificial
  Recombination region of the expression clone resulting from pLenti6/UbC/V5 DEST x entry clone
ttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccactagtccagtgtggtggaattctgcagatatcaacaagtttgtacaaaaaagcaggctnnacccagctttcttgtacaaagtggttgatatccagcacagtggcggccgctcgagtctagagggcccgcggttcgaaggtaagcctatccctaaccctctcctcggtctcgattctacgcgtaccggttagtaatgagttt

SEQ ID NO: 152 - DNA, 293 bp, Artificial
  Cloning region of pLenti6/V5 D Topo
tcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattgatcccttcaccatgaagggctcgagtctagagggcccgcggttcgaaggtaagcctatccctaaccctctcctcggtctcgattctacgcgtaccggttagtaatgagtttggaa

SEQ ID NO: 153 - DNA, 310 bp, Artificial
  Recombination region of pcDNA6.2/V5 DEST
caaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctntagnacccagctttcttgtacaaagtggttgatctagagggcccgcggttcgaaggtaagcctatccctaaccctctcctcggtctcgattctacgcgtaccggttagtaatgagtttaaacgggggaggctaactgaaacacg

SEQ ID NO: 154 - DNA, 379 bp, Artificial
  Recombination region of pcDNA6.2/GFP DEST
caaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctntagnacccagctttcttgtacaaagtggttgatctagagggccccgcggctagcaaaggagaagaacttttcactggaggtgtcccaattcttgttgaattagatggtgatgttaatgggcacaaattttctgtcagtggagagggtgaaggtgatgctacatacggaaagcttacccttaaatttatttgcactactggaaaactacctgtt

SEQ ID NO: 155 - DNA, 5 bp, Artificial
  Topoisomerase recognition site
ncctt

SEQ ID NO: 156 - DNA, 7 bp, Artificial
  Topoisomerase recognition site for type IA E. coli topoisomerase III
gcaactt

SEQ ID NO: 157 - DNA, 7 bp, Artificial
  Overlap region; bases 6-12 in the core region
tttatac

SEQ ID NO: 158 - DNA, 7 bp, Artificial
  Consensus sequence
nnnatac

SEQ ID NO: 159 - DNA, 7 bp, Artificial
  Kozak consensus sequence
nnnatgg

SEQ ID NO: 160 - DNA, 17 bp, Artificial
  Proposed Reverse PCR primer sequence
tgagctgctgccacaaa

SEQ ID NO: 161 - DNA, 7 bp, Artificial
  Seven base pair inverted repeat region
caacttt

SEQ ID NO: 162 - DNA, 7 bp, Artificial
  Seven base pair inverted repeat region
aaagttg

SEQ ID NO: 163 - DNA, 4 bp, Artificial
  PCR forward primer addition
cacc

SEQ ID NO: 164 - DNA, 4 bp, Artificial
  Overhang in cloning vector
gtgg

SEQ ID NO: 165 - protein, 7 aa, Artificial
  C-terminal polyhistidine tag and free carboxyl group
HHHHHHX

(19) United States
(12) Patent Application Publication
(10) Pub. No.: US 2004/0219516 A1
(43) Pub. Date: Nov. 4, 2004

(54) Viral vectors containing recombination sites

(75) Inventors: Robert P. Bennett, Encinitas, CA (US); Peter J. Welch, San Diego, CA (US); Steven Harwood, Oceanside, CA (US); Knut Madden, Carlsbad, CA (US); Kenneth Frimpong, San Diego, CA (US); Kenneth E. Franke, Davis, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/622,088

(22) Filed: Jul. 18, 2003

Related U.S. Application Data
(60) Provisional application No. 60/474,940, filed on Jun. 3, 2003.
(60) Provisional application No. 60/427,231, filed on Nov. 19, 2002.
(60) Provisional application No. 60/456,496, filed on Mar. 24, 2003.
(60) Provisional application No. 60/398,617, filed on Jul. 26, 2002.
(60) Provisional application No. 60/396,335, filed on Jul. 18, 2002.

(51) Int. Cl. C12Q 1/70; C07H 21/04; C07K 14/155

(52) U.S. Cl. 435/5; 435/69.3; 435/320.1; 435/456; 435/325; 536/23.72; 530/350

(57) ABSTRACT

The present invention provides compositions and methods for the construction of nucleic acids comprising all or portion of a viral genome. Nucleic acid molecules of the invention may be constructed to contain multiple recombination and/or topoisomerase recognition sites. The compositions include vectors having multiple recombination sites with unique specificity that contain all or a portion of a viral genome. The methods permit the insertion of a sequence of interest into a viral genome using recombinational and/or topoisomerase-mediated cloning. The present invention also provides methods of constructing recombinant virus, methods of expressing polypeptides, and methods of expressing fusion polypeptides.

### BACKGROUND OF THE INVENTION

1. Field of the Invention

The present invention relates to the fields of biotechnology and molecular biology. In particular, the present invention relates to nucleic acids comprising multiple recombination sites and comprising all or a portion of a viral genome as well as viruses and/or plasmids containing multiple recombination sites and their uses.

2. Related Art

Recombinant viruses are currently used in wide variety of applications. Viruses may be used for medical applications, for example, in gene therapy applications and/or as vaccines. Viruses may also be used in biotechnology applications, for example, as vectors to clone nucleic acids of interests and/or to produce proteins. Examples of recombinant viruses that have been used include, but are not limited to, herpes viruses (see, for example, U.S. Pat. No. 5,672,344, issued to Kelly, et al), pox viruses such as vaccinia virus (see, for example, Moss, et al., 1997, in Current Protocols in Molecular Biology, Chapters 16.15-16.18, John Wiley & Sons), papilloma viruses (see, for example, U.S. Pat. No. 6,342,224, issued to Bruck, et al.), retroviruses (see, for example U.S. Pat. No. 6,300,118, issued to Chavez, et al.), adenoviruses (see, for example, U.S. Pat. No. 6,261,807, issued to Crouzet, et al.), adeno-associated viruses (AAV, see for example, U.S. Pat. No. 5,252,479, issued to Srivastava), and coxsackie viruses (see, for example, U.S. Pat. No. 6,323,024).

When the viral nucleic acid is not infectious—for example, pox viruses—construction of recombinant viruses may involve in vivo homologous recombination in a virus-infected cell between the viral genome and concomitantly transfected plasmid bearing a sequence of interest flanked by viral sequences. When the viral nucleic acid is infectious—for example, adenovirus—a modified viral nucleic acid may be prepared and transfected into a host cell. Either methodology requires the preparation of a nucleic acid molecule containing a sequence of interest and some or all of the viral sequence. The preparation of this nucleic acid molecule may be a time-consuming, laborious process.

Adenoviruses are non-enveloped viruses with a 36 kb DNA genome that encodes more than 30 proteins. At the ends of the genome are inverted terminal repeats (ITRs) of approximately 100-150 base pairs. A sequence of approximately 300 base pairs located next to the 5′-ITR is required for packaging of the genome into the viral capsid. The genome as packaged in the virion has terminal proteins covalently attached to the ends of the linear genome.

The genes encoded by the adenoviral genome are divided into early and late genes depending upon the timing of their expression relative to the replication of the viral DNA. The early genes are expressed from four regions of the adenoviral genome termed E1-E4 and are transcribed prior to onset of DNA replication. Multiple genes are transcribed from each region. Portions of the adenoviral genome may be deleted without affecting the infectivity of the deleted virus. The genes transcribed from regions E1, E2, and E4 are essential for viral replication while those from the E3 region may be deleted without affecting replication. The genes from the essential regions can be supplied in trans to allow the propagation of a defective virus. For example, deletion of the E1 region of the adenoviral genome results in a virus that is replication defective. Viruses deleted in this region are grown on 293 cells that express the viral E1 genes from the genome of the cell.

In addition to permitting the construction of a safer, replication-defective viruses, deletion and complementation in trans of portions of the adenoviral genome and/or deletion of non-essential regions make space in the adenoviral genome for the insertion of heterologous DNA sequences. The packaging of viral DNA into a viral particle is size restricted with an upper limit of approximately 38 kb of DNA. In order to maximize the amount of heterologous DNA that may be inserted and packaged, viruses have been constructed that lack all of the viral genome except the ITRs and packaging sequence (see, U.S. Pat. No. 6,228,646). All of the viral functions necessary for replication and packaging are provided in trans from a defective helper virus that is deleted in the packaging signal.

Recombinant adenoviruses have been used as a gene transfer vectors both in vitro and in vivo. Their principal attractions as a gene transfer vector are their ability to infect a wide variety of cells including dividing and non-dividing cells and their ability to be grown in cell culture to high titers. A number of systems to insert heterologous DNA into the adenoviral genome have been developed. The adenoviral genome has been inserted into a yeast artificial chromosome (YAC, see Ketner, et al., PNAS 91:6186-90, 1994). Mutations may be introduced into the genome by transfecting a mutation-containing plasmid into a yeast cell that contains the adenoviral YAC. Homologous recombination between the YAC and the plasmid introduces the mutation into the adenoviral genome. The adenoviral genome can be removed from the YAC by restriction digest and the genome released by restriction digest is infectious when transfected into host cells. A similar system using two plasmids has been developed in E. coli (see Crouzet, et al., PNAS 94:1414-1419, 1997, and U.S. Pat. No. 6,261,807). In this system, the adenoviral genome is introduced into a inc-P derived replicon. Mutations are introduced by homologous recombination with a plasmid containing a ColE1 origin of replication. The ITRs in the inc-P plasmid are flanked by a restriction site not present in the rest of the viral genome, thus, infectious DNA can be liberated from the plasmid by restriction digest.

A number of viruses containing recombination site sequences and/or encoding recombinases have been prepared. For example, the Cre recombinase has been expressed from recombinant adenovirus and used to excise fragments from a mouse genome that were flanked with lox sites (see, Wang, et al., PNAS 93:3932-3936, 1996). U.S. Pat. No. 6,156,497 describes a system for constructing adenoviral genomes utilizing a first nucleic acid having an ITR, packaging signal, DNA of interest, and recombination site and a second nucleic acid having a recombination site and an ITR to which is bound a terminal protein. In the presence of recombinase, the two molecules are joined to form an infectious viral DNA.

Baculoviruses are large, enveloped viruses that infect arthropods. Baculoviral genomes are double-stranded DNA molecules of approximately 130 kbp in length. Baculoviruses have gained widespread use as systems in which to express proteins, particularly proteins from eukaryotic organisms (e.g., mammals), as the insect cells used to culture the virus may more closely mimic the post-translational modifications (e.g., glycosylation, acylation, etc.) of the native organism.

Numerous expression systems utilizing recombinant baculoviruses have been developed. General methods for constructing recombinant baculoviruses for expression of heterologous proteins may be found in Piwnica-Worms, et al., (1997) Expression of Proteins in Insect Cells Using Baculovirus Vectors, in Current Protocols in Molecular Biology, Chapter 16, pp. 16.9.1 to 16.11.12, Ausubel, et al. Eds., John Wiley & Sons, Inc. Other expression systems are known, for example, U.S. Pat. No. 6,255,060, issued to Clark, et al. discloses a baculoviral expression system for expressing nucleotide sequences that include a tag. U.S. Pat. No. 5,244,805, issued to Miller, discloses a baculoviral expression system that utilizes a modified promoter not naturally found in baculoviruses. U.S. Pat. No. 5,169,784, issued to Summers, et al. discloses a baculoviral expression system that utilizes dual promoters (e.g., a baculoviral early promoter and a baculoviral late promoter). U.S. Pat. No. 5,162,222, issued to Guarino, et al. discloses a baculoviral expression system that can be used to create stable cells lines or infectious viruses expressing heterologous proteins from a baculoviral immediate-early promoter (i.e., IEN). U.S. Pat. No. 5,155,037, issued to Summers, et al. discloses a baculoviral expression system that utilizes insect cell secretion signal to improve efficiency of processing and secretion of heterologous genes. U.S. Pat. No. 5,077,214, issued to Guarino, et al. discloses the use of baculoviral early gene promoters to construct stable cell lines expression heterologous genes. U.S. Pat. No. 4,879,239, issued to Smith, et al. discloses a baculoviral expression system that utilizes the baculoviral polyhedrin promoter to control the expression of heterologous genes.

Various methods of constructing recombinant baculoviruses have been used. A frequently used method involves transfecting baculoviral DNA and a plasmid containing baculoviral sequences flanking a heterologous sequence. Homologous recombination between the plasmid and the baculoviral genome results in a recombinant baculovirus containing the heterologous sequences. This results in a mixed population of recombinant and non-recombinant viruses. Recombinant baculoviruses may be isolated from non-recombinant by plaque purification. Viruses produced in this fashion may require several rounds of plaque purification to obtain a pure strain. Methods to reduce the background of non-recombinant viruses produced by homologous recombination methods have been developed. For example, a linearized baculoviral genome containing a lethal deletion, BaculoGold™, is commercially available from BD Biosciences, San Jose, Calif. The lethal deletion is rescued by homologous recombination with plasmids containing baculoviral sequences from the polyhedrin locus.

Methods utilizing direct insertion of foreign sequences into a baculoviral genome are also known. For example, Peakman, et al. (Nucleic Acids Res 20(3):495-500, 1992) disclose the construction of baculoviruses having a lox site in the genome. Heterologous sequences may be moved into the genome by in vitro site-specific recombination between a plasmid having a lox site and the baculoviral genome in the presence of Cre recombinase. U.S. Pat. No. 5,348,886, issued to Lee, et al. discloses a baculoviral expression system that utilizes a bacmid (a hybrid molecule comprising a baculoviral genome and a prokaryotic origin of replication and selectable marker) containing a recombination site for Tn7 transposon. Prokaryotic cells carrying the bacmid are transformed with a plasmid having a Tn7 recombination site and with a plasmid expressing the activities necessary to catalyze recombination between the Tn7 sites. Heterologous sequences present on the plasmid are introduced into the bacmid by site-specific recombination between the Tn7 sites. The recombinant bacmid may be purified from the prokaryotic host and introduced into insect cells to initiate an infection. Recombinant viruses carrying the heterologous sequence are produced by the cells transfected with the bacmid.

The family Retroviridae contains three subfamilies: 1) oncovirinae; 2) spumavirinae; and 3) lentivirinae. Retroviruses (e.g., lentiviruses) are viruses having an RNA genome that replicate through a DNA intermediate. A retroviral particle contains two copies of the RNA genome and viral replication enzymes in a RNA-protein viral core. The core is surrounded by a viral envelop made up of virally encoded glycoproteins and host cell membrane. In the early steps of infection, retroviruses deliver the RNA-protein complex into the cytoplasm of the target cell. The RNA is reverse transcribed into double-stranded cDNA and a pre-integration complex containing the cDNA and the viral factors necessary to integrate the cDNA into the target cell genome is formed. The complex migrates to the nucleus of the target cell and the cDNA is integrated into the genome of the target cell. As a consequence of this integration, the DNA corresponding to the viral genome (and any heterologous sequences contained in the viral genome) is replicated and passed on to daughter cells. This makes it possible to permanently introduce heterologous sequences into cells.

A wide variety of retroviruses are known, for example, leukemia viruses such as a Moloney Murine Leukemia Virus (MMLV) and immunodeficiency viruses such as the Human Immunodeficiency Virus (HIV). Representative examples of retroviruses include, but are not limited to, the Gibbon Ape Leukemia virus (GALV), Avian Sarcoma-Leukosis Virus (ASLV), which includes but is not limited to Rous Sarcoma Virus (RSV), Avian Myeloblastosis Virus (AMV), Avian Erythroblastosis Virus (AEV) Helper Virus, Avian Myelocytomatosis Virus, Avian Reticuloendotheliosis Virus, Avian Sarcoma Virus, Rous Associated Virus (RAV), and Myeloblastosis Associated Virus (MAV).

Retroviruses have found widespread use as gene therapy vectors. To reduce the risk of transmission of the gene therapy vector, gene therapy vectors have been developed that have modifications that prevent the production of replication competent viruses once introduced into a target cell. For example, U.S. Pat. No. 5,741,486 issued to Pathak, et al. describes retroviral vectors comprising direct repeats flanking a sequence that is desired to be deleted (e.g., a cis-acting packing signal) upon reverse transcription in a host cell. Deletion of the packing signal prevents packaging of the recombinant viral genome into retroviral particles, thus preventing spread of retroviral vectors to non-target cells in the event of infection with replication competent viruses. U.S. Pat. Nos. 5,686,279, 5,834,256, 5,858,740, 5,994,136, 6,013,516, 6,051,427, 6,165,782, and 6,218,187 describe a retroviral packaging system for preparing high titer stocks of recombinant retroviruses. Plasmids encoding the retroviral functions required to package a recombinant retroviral genome are provided in trans. The packaged recombinant retroviral genomes may be harvested and used to infect a desired target cell.

The family Herpesviridae contains three subfamilies 1) alphaherpesvirinae, containing among others human herpesvirus 1; 2) betaherpesvirinae, containing the cytomegaloviruses; and 3) gammaherpesvirinae. Herpesviruses are enveloped DNA viruses. Herpesviruses form particles that are approximately spherical in shape and that contain one molecule of linear dsDNA and approximately 20 structural proteins. Numerous herpesviruses have been isolated from a wide variety of hosts. For example, U.S. Pat. No. 6,121,043 issued to Cochran, et al. describes recombinant herpesvirus of turkeys comprising a foreign DNA inserted into a non-essential region of the herpesvirus of turkeys genome; U.S. Pat. No. 6,410,311 issued to Cochran, et al. describes recombinant feline herpesvirus comprising a foreign DNA inserted into a region corresponding to a 3.0 kb EcoRI-SalI fragment of a feline herpesvirus genome, U.S. Pat. No. 6,379,967 issued to Meredith, et al., describes herpesvirus saimiri, (HVS; a lymphotropic virus of squirrel monkeys) as a viral vector; and U.S. Pat. No. 6,086,902 issued to Zamb, et al. describes recombinant bovine herpesvirus type 1 vaccines.

Herpesviruses have been used as vectors to deliver exogenous nucleic acid material to a host cell. In addition to the examples above, U.S. Pat. No. 4,859,587, issued to Roizman describes recombinant herpes simplex viruses, vaccines and methods, U.S. Pat. No. 5,998,208 issued to Fraefel, et al., describes a helper virus-free herpesvirus vector packaging system, U.S. Pat. No. 6,342,229 issued to O'Hare, et al., describes herpesvirus particles comprising fusion protein and their preparation and use and U.S. Pat. No. 6,319,703 issued to Speck describes recombinant virus vectors that include a double mutant herpesvirus such as an herpes simplex virus-1 (HSV-1) mutant lacking the essential glycoprotein gH gene and having a mutation impairing the function of the gene product VP 16.

RNA viruses, such as those of the families Flaviviridae and Togaviridae have also been used to deliver exogenous nucleic acids to target cells. For example, members of the genus alphavirus in the family Togaviridae have been engineered for the high level expression of heterologous RNAs and polypeptides (Frolov et al., Proc. Natl. Acad. Sci. U.S.A. 93: 11371-11377 (1996)). Alphaviruses are positive stranded RNA viruses. A single genomic RNA molecule is packaged in the virion. RNA replication occurs by synthesis of a full-length minus strand RNA intermediate that is used as a template for synthesis of positive strand genomic RNA as well for synthesis of a positive strand sub-genomic RNA initiated from an internal promoter. The sub-genomic RNA can accumulate to very high levels in infected cells making alphaviruses attractive as transient expression systems. Examples of alphaviruses are Sindbis virus and Semliki Forest Virus. Kunjin virus is an example of a flavivirus. Sub-genomic replicons of Kunjin virus have been engineered to express heterologous polypeptides (Khromykh and Westaway, J. Virol. 71: 1497-1505 (1997)). The genomic RNA of both flaviviruses and togaviruses are infectious; transfection of the naked genomic RNA results in production of infective virus particles.

Methods for constructing recombinant viruses are typically laborious and time consuming. There remains a need in the art for materials and methods for the rapid and precise and rapid construction of recombinant viruses containing a nucleic acid region of interest. This need and others are met by the present invention.

### BRIEF SUMMARY OF THE INVENTION

The present invention provides, in part, a nucleic acid molecule comprising all or a portion of a viral genome (e.g., an adenovirus genome, a baculovirus genome, a herpesvirus genome, a pox virus genome, an adeno-associated virus genome, a retrovirus genome, a flavivirus genome, a togavirus genome, an alphavirus genome, an RNA virus genome, etc.). Nucleic acid molecules of the invention may further comprise at least two recombination sites (e.g., three, four, five, six, seven, eight, nine, ten, etc.) that, in most instances, do not recombine with each other. In particular embodiments, the viral genome may be an adenoviral genome, a baculoviral genome, a retroviral genome (e.g., a lentiviral genome), an RNA virus genome or a herpesvirus genome. In some embodiments, the viral genome is not an adenoviral genome, is not a baculoviral genome, is not a retroviral genome (e.g., a lentiviral genome), and/or is not a herpesvirus genome. In some embodiments, the viral genome is not from a virus that infects prokaryotic organisms. In some embodiments, one or more of the two or more recombination sites is not a lox site. In some embodiments, nucleic acid molecules comprising one or more sequences of interest are combined with nucleic acid molecules comprising all or a portion of a viral genome using a recombination system that does not use a recombination system derived from a transposon (e.g., Tn7). In some embodiments, nucleic acid molecules of the invention may not contain a lox site.

Optionally, nucleic acid molecules of the invention may comprise one or more features that confer desired characteristics on the nucleic acid molecules. Examples of features include, but are not limited to, promoters, viral terminal repeats (e.g., long terminal repeats (LTRs)), splice sites (e.g., 5′-splice doneor sites and/or 3′-splice acceptor sites), packaging signals, nucleic acid sequences responsive to one or more viral proteins (e.g., rev response element (RRE)), recognition sites (e.g., restriction enzyme recognition sites), recombination sites, sequences encoding marker proteins or polypeptides (e.g., antibiotic resitance enzymes, toxic proteins, etc.), sequences encoding epitopes recognizable by an antibody (e.g., V5 epitope), origins of replication (which may function in prokaryotic and/or eukaryotic cells), intervening sequences (e.g., β-globin intron), internal ribosome entry sequences (IRES), and polyadenylation signals (e.g., SV40 polyadenylation signal). Additional examples of such nucleic acid molecules include those which contain at least (1) one or more (e.g., one, two, three, four, five, six, seven, eight, nine, etc.) component of one or more of the vectors represented in FIGS. 1, 2, 4, 5, 6, 7, 8, 9, 10, 15, 18, 20, 22, 34, 36, 37, 49, 57, 58, 59, 60, 69, 70, 71 or 72; or (2) one or more components of such vectors which confer the same or similar feature upon a nucleic acid molecule. As a specific example, a nucleic acid molecule of the invention may be a vector which comprises, in addition to recombination sites, at least one blasticidin resistance marker (see, e.g., FIG. 22), at least one GP64 promoter (see, e.g., FIG. 22), at least one RSV promoter (see, e.g., FIG. 36A), at least one beta-globin intron (see, e.g., FIG. 37A), at least one ampicillin resistance marker (see, e.g., FIG. 37A), and at least one bacterial origin of replication (see, e.g., FIG. 37A). In most instances, the combinations of components selected for inclusion in a nucleic acid molecule will be designed to provide activities intended for a particular use. For example, a vector which is capable of expressing a nucleic acid insert in more than one type of eukaryotic cells (e.g., human cells and insect cells) and is replicable in prokaryotic cells (e.g., E. coli cells) may be desired. Thus, the components which are selected for inclusion in nucleic acid molecules of the invention will typically be determined by the particular use for which it is designed. The invention further includes methods for making and using such nucleic acid molecules as described, for example, elsewhere herein.

Viruses produced using nucleic acids of the present invention may be used as viral vectors (e.g., viruses containing at least one heterologous sequence), for example, to deliver exogenous sequences to cells or organisms. The present invention also contemplates compositions comprising nucleic acids and/or viruses of the invention, as well as methods of making and using such nucleic acids, viruses, and compositions.

Viral genomes that may be used with the present invention (e.g., retroviral genomes, adenoviral genomes, herpesvirus genomes, genomes of RNA viruses, and/or baculoviral genomes) may be wild type or may contain one or more mutations, insertions and/or deletions. In some embodiments, viral genomes for use in the practice of the present invention may be adenoviral genomes containing one or more deletions. Deleted adenoviral genomes may be deleted in one or more regions of the genome. Regions of the adenoviral genome that may be deleted, include, but are not limited to, the E1 and E3 regions.

Adenoviral genomes for use in the present invention may be infectious. In some embodiments, an adenoviral genome may be infectious when introduced into cells expressing one or more adenoviral proteins (e.g., the E1 proteins as in 293 cells). In some embodiments, a viral genome used in the invention is an Ad5 viral genome.

Baculoviral genomes that may be used in the practice of the present invention may be entire genomes or may contain one or more deletions, for example, at the polyhedrin locus. Suitable genomes include those from any virus in the family Baculoviridae. Suitable viral genomes include, but are not limited to, those from occluded baculoviruses (e.g., nuclear polyhedrosis viruses (NPV) such as Autographa californica nuclear polyhedrosis virus (AcMNPV), Choristoneura fumiferana MNPV (CfMNPV), Mamestra brassicae MNPV (MbMNPV), Orgyia pseudotsugata MNPV (OPMNPV), Bombyx mori S Nuclear Polyhedrosis Virus (BmNPV), Heliothis zea SNPV (HzSnpv), and Trichoplusia ni SNPV (TnSnpv) and granulosis viruses (GV) (e.g., Plodia interpunctella granulosis virus (PiGV), Trichoplusia ni granulosis virus (TnGV), Pieris brassicae granulosis virus (PbGV), Artogeia rapae granulosis virus (ArGV), and Cydia pomonella granulosis virus (CpGV)). Suitable genomes also include, but are not limited to, those from non-occluded baculoviruses (NOB) (e.g., Heliothis zea NOB (HzNOB), Oryctes rhinoceros virus), etc.

In some embodiments, viral genomes for use in the practice of the present invention may be retroviral genomes containing one or more deletions. Deleted retroviral genomes may be deleted in one or more regions of the genome. Regions of the retroviral genome that may be deleted, include, but are not limited to, the gag, pol, env, and rev regions. In some embodiments, a retroviral genome may be deleted of all retroviral sequences except the 5′-LTR, 3′-LTR and packaging signal (Ψ). In some embodiments, retroviral genomes of the present invention may comprise one or more heterologous sequences (e.g., sequences derived from another organism such as another virus). In a particular embodiment, nucleic acid molecules of the invention may comprise a deleted retroviral genome and may also comprise one or more heterologous sequences that may be promoter sequences. In some embodiments, nucleic acid molecules of the invention may comprise a deleted retroviral genome and may further comprise the CMV promoter.

In some embodiments, nucleic acid molecules of the present invention may be in the form of plasmids and/or bacmids comprising one or more origins of replication and, optionally, one or more selectable markers. In certain embodiments, nucleic acid molecules of the invention (e.g., plasmids and/or bacmids) may comprise one or more recognition sequences (e.g., recombination sequences, topoisomerase sequences, restriction enzyme sequences, etc.), which may be recognized by the same or different enzymes. For example, in some embodiments, plasmids comprising all or a portion of the viral genome may comprise one or more recombination sites that may not recombine with each other. In certain embodiments, nucleic acid molecules of the invention (e.g., plasmids and/or bacmids) may comprise restriction enzyme recognition sequences, which may be recognized by the same or different restriction endonucleases, arranged such that digestion with one or more restriction enzymes that recognize the recognition sequences produces a linear molecule comprising the viral genome. In some embodiments, digestion with a restriction enzyme may remove a portion of plasmid and/or bacmid. For example, in some embodiments, plasmids comprising all or a portion of the adenoviral genome may be digested so as to remove the origin of replication and, optionally, the selectable marker from the plasmid. In another example, a nucleic acid molecule comprising all or a portion of a baculoviral genome may be digested with a restriction enzyme that linearizes the baculoviral genome, for example, by cleaving the nucleic acid molecule at a recognition site located between two recombination sites (see FIG. 20). In embodiments of this type, the baculoviral genome may be re-circularized by recombination with a second nucleic acid molecule having recombination sites that are capable of recombining with those in the nucleic acid molecule comprising all or a portion of the baculoviral genome. In particular embodiments, the restriction enzyme recognition sites may be recognized by two different restriction enzymes. Thus, the invention includes methods for selecting recombinant nucleic acid molecules (e.g., recombinant baculoviral vectors). The method may comprise recombining a first nucleic acid molecule, which may be linearized, with a second nucleic acid molecule to produce a circularized molecule that is capable of replicating when introduced into a suitable host cell. The method may also comprise selecting against re-circularized first nucleic acid molecule that did not undergo recombination with the second nucleic acid molecule. In some embodiments, the first nucleic acid molecule may be a linearized baculoviral genome.

A nucleic acid sequence of interest may be inserted into the nucleic acid molecule of the invention using recombinational cloning techniques. In some embodiments, a nucleic acid molecule of the invention may comprise a heterologous promoter (e.g., the CMV promoter) and one or more recombination sites arranged such that a nucleic acid sequence of interest can be inserted into the nucleic acid molecule of the invention by recombination with one or more of the recombination sites and, after insertion, the nucleic acid sequence of interest may be operably linked to the heterologous promoter. In some embodiments, a nucleic acid molecule of the invention may have a heterologous promoter located adjacent to two recombination sites that do not recombine with each other. A nucleic acid sequence of interest can be inserted into the nucleic acid molecule of the invention between the two recombination sites and may then be operably linked to the heterologous promoter.

Any nucleic acid sequence of interest may be placed between the recombination sites present in the nucleic acids of the present invention. For example, the nucleic acid sequence between the recombination sites may encode one or more polypeptides of interest. The viral vectors of the present invention may be used to express libraries of sequences, for example, genomic libraries or cDNA libraries. A sequence of interest may be a sequence coding for a polypeptide or may be a sequence that does not encode a polypeptide. Examples of sequences of interest that do not encode a polypeptide include, but are not limited to, sequences encoding tRNA sequences (e.g., suppressor tRNA sequences), sequences encoding ribozyme sequences, promoter sequences, enhancer sequences, repressor sequences and the like. In some embodiments, the sequence of interest may encode one or more polypeptides and may further comprise one or more stop codons in the sequence. In some embodiments, the nucleic acid between the recombination sites comprises at least one selectable marker. In some embodiments, the sequence of interest comprises a sequence encoding at least one suppressor tRNA and/or at least one aminoacyl-tRNA synthetase.

In some embodiments, the present invention provides nucleic acid molecules comprising all or a potion of more than one viral genome. For example, a nucleic acid molecule of the invention may comprise all or a portion of a first viral genome (e.g., a retroviral genome) and all or a portion of one or more additional viral genomes (e.g., an adenoviral genome, a baculoviral genome, a herpesvirus genome, a pox virus genome, an RNA virus genome, etc). In some embodiments, the nucleic acid molecules of the invention may comprise nucleic acid sequences from more than one virus. Nucleic acid molecules of this type may comprise viral sequences that permit the replication of the nucleic acid in more than one type of organism (e.g., mammalian cells and insect cells) and may also include sequences capable of functioning as transcriptional regulatory sequences (e.g., promoters, enhancers, etc.) that function in more than one cell type. For example, one viral sequence may function as a promoter in one cell type (e.g., mammalian) while another viral sequence may function as a promoter in another cell type (e.g., insect).

In another aspect, the present invention provides a method of constructing a nucleic acid molecule comprising all or a portion of one or more viral genomes (e.g., a recombinant virus such as a viral vector). In some embodiments, methods of the invention may comprise providing at least a first nucleic acid molecule comprising all or a portion of at least one viral genome and at least a first and a second recombination site that do not recombine with each other. Methods of the invention may also entail contacting at least a first nucleic acid molecule with at least a second nucleic acid molecule comprising at least one sequence of interest flanked by at least a third and a fourth recombination site under conditions causing recombination between the first and third recombination site and between the second and fourth recombination site. In some embodiments, the viral genome may be an adenoviral genome, for example, an Ad5 adenoviral genome. In some embodiments, the viral genome may be a baculoviral genome, for example, an Autographa califomica multiple nuclear polyhedrosis virus (ACMNPV) genome. In some embodiments, the viral genome may be a retroviral genome (e.g., a lentiviral genome).

In some embodiments, a first nucleic acid molecule comprising all or a portion of a viral genome for use in the methods of the invention may be a plasmid that may comprise an origin of replication and a selectable marker. The first nucleic acid molecule may, optionally, contain two restriction enzyme recognition sequences, which may be for the same or different restriction enzymes, arranged such that digestion with the appropriate restriction enzyme or restriction enzymes produces a linear molecule comprising the viral genome (e.g., adenoviral genome) and lacking the origin of replication and/or the selectable marker.

In some embodiments, the first nucleic acid molecule may comprise at least a first and a second recombination site, which may or may not recombine with each other, and the portion of the first nucleic acid molecule between the first and second recombination sites may comprise a sequence encoding at least one selectable marker. In some embodiments, a second nucleic acid molecule, which may or may not comprise viral sequences, may comprise at least a third and a fourth recombination site and a sequence of interest between the third and fourth recombination site. The sequence of interest may be any sequence, for example, a sequence encoding a polypeptide or a sequence of a functional RNA (e.g., a suppressor tRNA sequence). In some embodiments, the first and second nucleic acid molecules may be contacted with one or more recombination proteins such that the sequence of interest is transferred to the first nucleic acid molecule resulting in a first nucleic acid molecule comprising all or a portion of a viral genome and further comprising at least one sequence of interest (e.g., a polypeptide coding region, a tRNA coding sequence etc.). The present invention also contemplates compositions comprising a nucleic acid molecule comprising all or a portion of a viral genome and further comprising at least one sequence of interest, as well as methods of making and using such nucleic acids and compositions. In some embodiments, the sequence of interest may be a tRNA coding sequence.

In some embodiments, a first nucleic acid molecule comprising all or a portion of a viral genome for use in the methods of the invention may be a bacmid that may comprise an origin of replication and a selectable marker. The first nucleic acid molecule may, optionally, contain a restriction enzyme recognition sequence, located such that digestion with the appropriate restriction enzyme produces a linear molecule comprising the viral genome (e.g., baculoviral genome). In some embodiments, the first nucleic acid molecule may comprise at least a first and a second recombination site, which may or may not recombine with each other, and the recognition site for the restriction enzyme may be located between the recombination sites. Optionally, the portion of the first nucleic acid molecule between the first and second recombination sites may comprise a sequence encoding at least one selectable marker. In some embodiments, a second nucleic acid molecule, which may or may not comprise viral sequences, may comprise at least a third and a fourth recombination site and the sequence between the third and fourth recombination site comprises a sequence of a functional RNA (e.g., a suppressor tRNA sequence). In some embodiments, the first and second nucleic acid molecules may be contacted with one or more recombination proteins such that the functional sequence (e.g., a sequence encoding a suppressor tRNA sequence) is transferred to the first nucleic acid molecule resulting in the first nucleic acid molecule re-circularizing and further comprising at least one functional sequence (e.g., a sequence encoding a tRNA). The present invention also contemplates compositions comprising a nucleic acid molecule comprising all or a portion of a viral genome and further comprising at least one functional sequence, as well as methods of making and using such nucleic acids and compositions.

The present invention also provides, in part, materials and methods for joining or combining two or more (e.g., two, three, four, five, seven, ten, twelve, fifteen, twenty, thirty, fifty, seventy-five, one hundred, two hundred, etc.) nucleic acid segments and/or nucleic acid molecules by a recombination reaction between recombination sites—at least one of which is present on each molecule and/or segment—in order to construct a nucleic acid molecule comprising all or a portion of a viral genome (e.g., a retroviral genome, an adenoviral genome and/or a baculoviral genome). In embodiments of this type, one or more nucleic acid segments and/or nucleic acid molecules may comprise viral nucleic acid sequences. Such recombination reactions to join multiple nucleic acid segments and/or nucleic acid molecules according to the invention may be conducted in vivo (e.g., within a cell, tissue, organ or organism) or in vitro (e.g., cell-free systems). The invention also relates to hosts and host cells comprising the viral vectors and/or nucleic acid molecules of the invention. The invention also relates to kits for carrying out methods of the invention, and to compositions for carrying out methods of the invention, as well as to compositions used in and made while carrying out the methods of the invention.

Nucleic acid molecules prepared by methods of the invention may be used for any purpose known to those skilled in the art. For example, nucleic acid molecules of the invention may be used to express proteins or peptides encoded by these nucleic acid molecules and may also be used to create novel fusion proteins by expressing different nucleic acid sequences linked by the methods of the invention. Nucleic acids of the invention may also be used to produce RNA molecules that are not translated into polypeptides or proteins, for example, tRNAs, anti-sense molecules, interfering RNA and/or ribozymes.

Nucleic acid molecules of the invention may be used as part of a system to generate replication-defective viral particles. For example, nucleic acid molecules of the invention may be packaged into a viral particle using techniques known in the art. Packaging may be accomplished by providing requisite packaging activities in trans, for example, on a different nucleic acid molecule and/or in the genome of a cell. In a particular example, nucleic acid molecules of the invention may be used to construct a replication-defective lentivirus. In a particular embodiment, nucleic acid molecules of the invention may comprise lentiviral long terminal repeats and packaging signal and other activities required to package the nucleic acid molecule of the invention may be provided in trans, for example, may be expressed from one or more plasmids.

In some aspects, methods of the present invention may comprise introducing a nucleic acid molecule of the invention into a cell or population of cells and detecting the presence or absence of the nucleic acid molecule. Such detection may be accomplished, for example, by detecting the presence or absence of one or more selectable marker present on the nucleic acid molecule. Optionally, a selectable marker may be a nucleic acid sequence encoding a polypeptide having β-lactamase activity. Detection may be accomplished by contacting a cell or population of cells with a fluorogenic substrate for β-lactamase activity and detecting fluorescence of the cell or population of cells. In a specific embodiment, the fluorogenic substrate may be CCF2/AM and fluorescence may be detected by illuminating the cell with light having a wavelength of 405 nm and detecting fluorescence at a wavelength of approximately 450 nm and at a wavelength of approximately 520 nm. Methods may also comprise comparing the amount of fluorescence observed at 450 nm and 520 mn, for example, by determining a ratio between the observed fluorescence amounts. Methods may also comprise physically separating cells having a desired nucleic acid molecule by fluorescent activated cell sorting (FACS).

The present invention provides methods for infecting, transfecting, transducing and/or otherwise introducing the nucleic acid molecules of the invention into host cells and, optionally, expressing one or more sequences of interest present on the nucleic acid molecule of the invention. Suitable host cells may be dividing or non-dividing cells. In a particular embodiment, host cells using in connection with the methods of the invention are non-dividing cells. For example, one or more nucleic acid molecule of the invention may be introduced into one or more non-dividing cells. One or more of the nucleic acid molecules may comprise a sequence of interest that may encode a polypeptide or an untranslated RNA. The methods of the invention may result in the production in the non-dividing cells of a polypeptide or untranslated RNA encoded by the sequence of interest. Nucleic acid molecules of the invention for use in the expression of a sequence of interest in a non-dividing cell may comprise one or more sequences from one or more viruses, for example, from an adenovirus and/or a lentivirus. A nucleic acid molecule of the invention for expression of a sequence of interest in a non-dividing cell may comprise one or more adenoviral sequences. A nucleic acid molecule of the invention for expression of a sequence of interest in a non-dividing cell may comprise one or more lentiviral sequences.

Recombination sites for use in the methods and/or compositions of the invention may be any recognition sequence on a nucleic acid molecule that participates in a recombination reaction mediated or catalyzed by one or more recombination proteins. In those embodiments of the present invention utilizing more than one (e.g., two, three, four, five, seven, ten, twelve, fifteen, twenty, thirty, fifty, etc.) recombination sites, such recombination sites may be the same or different and may recombine with each other or may not recombine or not substantially recombine with each other. Recombination sites contemplated by the invention also include mutants, derivatives or variants of wild-type or naturally occurring recombination sites. Desired modifications can also be made to the recombination sites to include changes to the nucleotide sequence of the recombination site that cause desired sequence changes to the transcription product (e.g., mRNA, tRNA, ribozyme, etc.) and/or desired amino acid changes in the translation product (e.g., polypeptide or protein) when transcription occurs across the modified recombination site.

Preferred recombination sites used in accordance with the invention include att sites, frt sites, dif sites, psi sites, cer sites, and lox sites or mutants, derivatives and variants thereof (or combinations thereof). Recombination sites contemplated by the invention also include portions of such recombination sites. Depending on the recombination site specificity used, the invention allows directional linking of nucleic acid molecules to provide desired orientations of the linked molecules or non-directional linking to produce random orientations of the linked molecules.

In certain embodiments, recombination proteins used in the practice of the invention comprise one or more proteins selected from the group consisting of Cre, Int, IHF, Xis, Flp, Fis, Hin, Gin, Cin, Tn3 resolvase, TndX, XerC, XerD, and ΦC31. In specific embodiments, the recombination sites comprise one or more recombination sites selected from the group consisting of lox sites; psi sites; dif sites; cer sites; fit sites; att sites; and mutants, variants, and derivatives of these recombination sites that retain the ability to undergo recombination.

In a specific aspect, the invention provides nucleic acid molecules and/or viral vectors that permit controlled expression of fusion polypeptides by suppression of one or more stop codons. According to the invention, a nucleic acid molecule, which may be any nucleic acid molecule, for example, a plasmid and/or a nucleic acid molecule comprising all or a portion of a viral genome and/or a viral vector produced by the methods of the invention, may comprise a sequence of interest that may comprise one or more stop codons (e.g., TAG, TAA, and/or TGA) that may be suppressed. In embodiments of this type, mRNA is transcribed from the nucleic acid molecule. The transcribed mRNA molecule comprises at least a first coding sequence corresponding to the sequence of interest and at least one additional sequence containing a second coding region separated from the first coding sequence by a stop codon. Suppression of the stop codon allows expression of both the first and second coding sequences in a single polypeptide molecule. The nucleic acid sequence corresponding to the additional sequence may be contained on the sequence of interest or may be contained in a recombination site or on the nucleic acid molecule. One or more suppressor tRNA molecules may be provided, for example, from any nucleic acid molecule such as a plasmid, a nucleic acid molecule comprising all or a portion of a viral genome and/or a viral vector of the invention.

Some embodiments of the present invention allow selective or controlled fusion protein expression by varying the suppression of selected stop codons. For example, a nucleic acid molecule, which may be a viral vector of the invention, may comprise three coding regions of interest separated by regions comprising stop codons. One or more of the coding regions of interest may be flanked by recombination sites. By suppressing the stop codon between the first and second coding regions a fusion polypeptide may be produced comprising amino acids encoded by the first and second coding region but not containing the amino acids encoded by the third region. Thus, use of different stop codons and variable control of suppression allows production of various fusion proteins or portions thereof encoded by all or different portions of the nucleic acid sequence of interest. In some embodiments, one or more of the coding regions in the sequence of interest may encode a polypeptide that comprises a sequence (preferably an N-terminal and/or a C-terminal tag sequence) encoding all or a portion of one or more of the following: the Fc portion of an immunoglobin, an antibody, a β-glucuronidase, a β-lactamase, a β-galactosidase, a fluorescent protein (e.g., green fluorescent protein, yellow fluorescent protein, red fluorescent protein, cyan fluorescent protein, etc.), a transcription activation domain, a protein or domain involved in translation, protein localization tag, a protein stabilization or destabilization sequence, a protein interaction domains, a binding domain for DNA, a protein substrate, a purification tag (e.g., an epitope tag, maltose binding protein, a six histidine tag, glutathione S-transferase, etc.), and an epitope tag.

In one aspect, a stop codon may be included anywhere within the sequence of interest or within a recombination site contained by nucleic acid molecules, which may be nucleic acid molecules comprising all or a portion of a viral genome. Preferably, stop codons are located at or near the termini of the sequence of interest, although stop codons may be included internally within the sequence. In another aspect, the sequence of interest may comprise the coding sequence of all or a portion of a target gene or open reading frame (ORF) of interest wherein the coding sequence is followed by a stop codon. The stop codon may then be followed by a recombination site allowing joining the sequence of interest to another nucleic acid molecule, which may be a nucleic acid molecule comprising all or a portion of a viral genome. After joining the sequence of interest with the nucleic acid molecule to form a recombinant nucleic acid molecule, the stop codon may be optionally suppressed by a suppressor tRNA molecule. In some embodiments of this type, one or more genes coding for one or more suppressor tRNA molecules (that may be the same or different) may be provided on the same nucleic acid molecule, or on another nucleic acid molecule. One or more genes coding for one or more suppressor tRNA molecules (that may be the same or different) may be provided on a different nucleic acid molecule, for example, a viral genome, a plasmid, a bacmid, a cosmid, a BAC, a YAC, a chromosome of the host cell into which the nucleic acid molecule of the invention is inserted, or any other nucleic acid molecule known to those skilled in the art. In some embodiments, one or more sequences encoding suppressor tRNAs may be provided on a nucleic acid molecule comprising all or a portion of a viral genome. In some embodiments, more than one copy (e.g., two, three, four, five, seven, ten, twelve, fifteen, twenty, thirty, fifty, etc. copies) of the gene encoding the suppressor tRNA may be provided. In some embodiments, the transcription of the suppressor tRNA may be under the control of a regulatable (e.g., inducible or repressible) promoter. In other embodiments, the transcription of the suppressor tRNA may be under the control of a constitutive promoter. When more than one gene encoding a suppressor tRNA is provided, the genes may be the same or different and may be expressed from the same or different promoters.

The sequence of interest may comprise a ORF of interest that may be provided with translation initiation signals (e.g., Shine-Delgamo sequences, Kozak sequences and/or IRES sequences) in order to permit the expression of a polypeptide from the ORF with a native N-terminus when the stop codon is not suppressed. Further, the sequence of interest may be constructed by recombinational cloning of two or more different sequences resulting in recombination sites within the sequence of interest. Recombination sites that reside between nucleic acid segments that encode components of fusion proteins may be designed either to not encode stop codons or to not encode stop codons in the fusion protein reading frame. A sequence of interest encoding a polypeptide may also be provided with a stop codon (e.g., a suppressible stop codon) at the 3′ end of the coding sequence. Similarly, when a fusion protein is produced from multiple nucleic acid segments (e.g., three, four, five, six, eight, ten, etc. segments), nucleic acids sequences that encode stop codons can be omitted between each nucleic acid segment and/or nucleic acids that encodes a stop codon can be positioned at the 3′ end of one or more of the segments and/or at the 3′ end of the 3′-most segment of the fusion protein coding region.

In some embodiments, a tag sequence may be provided at both the N- and C-termini of the gene of interest. Optionally, the tag sequence at the N-terminus may be provided with a stop codon and an ORF of interest may be provided with a stop codon and the tag at the C-terminus may be provided with a stop codon. The stop codons may be the same or different.

In some embodiments, the stop codon of the N-terminal tag is different from the stop codon of the ORF of interest. In embodiments of this type, suppressor tRNAs corresponding to one or both of the stop codons may be provided. When both are provided, each of the suppressor tRNAs may be independently provided on the same vector (e.g., plasmid, virus, etc.), on a different viral vector or other vector, or in the host cell genome. The suppressor tRNAs need not both be provided in the same way, for example, one may be provided on the vector contain the gene of interest while the other may be provided in the host cell genome.

Depending on the location of the expression signals (e.g., promoters), suppression of the stop codon(s) during expression allows production of a fusion peptide having the tag sequence at the N- and/or C-terminus of the expressed protein. By not suppressing the stop codon(s), expression of the sequence of interest without the N- and/or C-terminal tag sequence may be accomplished. Thus, the invention allows through recombination efficient construction of vectors (e.g., viral vectors) containing one or more ORFs (e.g., one, two, three, four, five, six, ten, or more ORFs) or other sequence of interest (e.g., untranslated sequences such as RNAi, tRNAs, ribozymes, etc.) for controlled expression of fusion proteins depending on the need. Those skilled in the art will appreciate that suppression is not 100% effective. Thus, under suppressing conditions a mixture of polypeptides is produced, the mixture comprising polypeptides that terminate at the stop codon and polypeptides that contain amino acid sequences encoded after the stop codon. For example, in the case discussed above where three coding regions are separated by two stop codons, under conditions designed to suppress both stop codons, a mixture containing various amounts of the polypeptide encoded by the first coding region plus a polypeptide encoded by the first and the second coding regions and a polypeptide containing amino acids of all three coding regions might be produced.

The present invention provides methods of making stable cell lines and cell lines made by the methods of the invention. Stable cell lines may incorporate one or more sequences of interest that may be incorporated into the genome of the cell or may be maintained extra-chromasomally. Optionally, a sequence of interest may include one or more stop codons, one or more of which may be located at or near the 3′ end of a coding sequence present in the sequence of interest. A stable cell line of the invention may be contacted with one or more nucleic acid molecules comprising all or a portion of a viral genome under conditions causing suppression of one or more of the stop codons present in the sequence of interest. A nucleic acid molecule comprising all or a portion of a viral genome may also comprise one or more copies (e.g., two, three, four, five, six, seven, eight, nine, ten, fifteen, twenty, twenty five, etc.) of a sequence that produces a suppressor tRNA. In the absence of the nucleic acid molecule expressing a suppressor tRNA, for example, a nucleic acid molecule comprising all or a portion of a viral genome and comprising one or more sequence encoding a suppressor tRNA, a stable cell line of the invention may express a polypeptide encoded by a sequence of interest such that the polypeptide has a native primary structure. In the presence of a suppressor expressing nucleic acid molecule, for example, a nucleic acid molecule comprising all or a portion of a viral genome and comprising one or more sequence encoding a suppressor tRNA, a stable cell line of the invention may express a fusion protein incorporating the polypeptide encoded by the sequence of interest and some additional peptide sequence. A stable cell line of the invention may also comprise a suppressor tRNA encoding sequence in the genome of the cell, which sequence may be under the control of a promoter that is inducible (e.g., inducible by a nucleic acid molecule comprising all or a portion of a viral genome or a polypeptide encoded by such a nucleic acid molecule). Thus, contacting the cell with a nucleic acid molecule comprising all or a portion of a viral genome may result in production of a suppressor tRNA and suppression of one or more stop codons present in a sequence of interest.

The sequences of interest to be incorporated in the viral vectors and/or nucleic acids molecules of the invention may comprise at least one open reading frame (ORF) (e.g., one, two, three, four, five, seven, ten, twelve, or fifteen ORFs). Such sequences may also comprise functional sequences (e.g., primer binding sites, transcriptional or translation sites or signals), termination sites (e.g., stop codons that may be optionally suppressed), origins of replication, and the like, and often will comprise sequences that regulate gene expression including transcriptional regulatory sequences and sequences that function as internal ribosome entry sites (IRES). Often, either the sequence of interest and/or the portions of the nucleic acid comprising the viral genome adjacent to the sequence of interest comprise sequences that function as a promoter. Either or both the sequence of interest and/or nucleic acid comprising all or a part of a viral genome may also comprise transcription termination sequences, selectable markers, restriction enzyme recognition sites, and the like.

In some embodiments, nucleic acid molecules of the invention comprising all or a portion of a viral genome may comprise two copies of the same selectable marker, each copy flanked by two recombination sites. In other embodiments, these molecules may comprise two different selectable markers each flanked by two recombination sites. In some embodiments, one or more of these selectable markers may be a negative selectable marker (e.g., ccdB, kicB, Herpes simplex thymidine kinase, cytosine deaminase, etc.).

In one aspect, the present invention provides a composition comprising a recombinant viral vector which encodes one or more suppressor tRNAs. Such compositions may comprise any number of additional components, for example, cells, media, buffers, proteins, lipids, and the like. In some embodiments, the viral vector may be an adenovirus. A viral vector may encode one or more suppressor tRNAs that recognize one of the stop codons selected from TAG, TGA, or TAA. In some embodiments, the viral vector encodes a plurality of suppressor tRNAs, for example, eight suppressor tRNAs that recognize the stop codon TAG.

In some embodiments, the present invention provides compositions comprising a nucleic acid molecule comprising all or a portion of at least one viral genome and further comprising at least two recombination sites that do not substantially recombine with each other ; and a polypeptide. Any polypeptide may be included in compositions of this type, for example, the polypeptide may be a viral envelop polypeptide. A composition of this type may be in the form of a particle comprising the nucleic acid molecule and the polypeptide. All or a portion of any viral genome may be included on the nucleic acid molecule, for example, the viral genomes may be a lentiviral genome, for example an HIV genome (such as HIV-1). A polypeptide suitable for compositions of this type is vesicular stomatitis virus G-protein.

In another aspect, the present invention provides host cells comprising a first nucleic acid sequence encoding a fusion polypeptide, wherein the sequence comprises at least a first coding region, and a second coding region separated by a sequence comprising a stop codon, and a second nucleic acid sequence comprising one or more suppressor tRNAs that suppresses the stop codon. In some embodiments, at least one of the first and/or second nucleic acid sequence is present on a nucleic acid molecule comprising all or a portion of at least one viral genome (e.g., an adenoviral genome). In some embodiments, the one or more suppressor tRNAs are expressed from a nucleic acid molecule comprising all or a portion of at least one viral genome (e.g., an adenoviral genome). A nucleic acid molecule may encode one or more suppressor tRNAs that recognizes one of the stop codons selected from TAG, TGA, or TAA. In some embodiments, the nucleic acid molecule may encode a plurality of suppressor tRNAs. In some embodiments, the nucleic acid molecule may encode eight suppressor tRNAs that recognize the stop codon TAG and may comprise all or a portion of an adenoviral genome.

In one aspect, the present invention provides a host cell comprising a nucleic acid molecule comprising all or a portion of at least one viral genome and further comprising at least two recombination sites that do not substantially recombine with each other. In some embodiments, at least one of the viral genomes may be a lentiviral genome (e.g., an HIV genome). In some aspects, a nucleic acid molecule may be stably integrated into the genome of the host cell. In some embodiments, at least one of the viral genomes may be an RNA virus genome (e.g., of the family Togaviridae or Flaviviridae such as an alphavirus, a Sindbis virus and a Kunjin virus).

In one aspect, the present invention provides a method of expressing a polypeptide. Such methods may comprise contacting a cell with a nucleic acid molecule comprising a sequence encoding the polypeptide operably linked to a promoter and a repressor sequence, wherein the nucleic acid molecule comprises all or a portion of a viral genome, contacting the cell with a nucleic acid molecule encoding a protein that binds to the repressor sequence; and incubating the cell under conditions sufficient to express the polypeptide. In embodiments of this type, the viral genome may be a lentiviral genome (e.g., an HIV). In some aspects, the repressor sequence may be the tetracycline operator sequence and the protein may be the tetracycline repressor protein and conditions sufficient to express the polypeptide comprise incubating the cell in the presence of a compound that reduces binding of the protein to the repressor sequence (e.g., tetracycline).

In another aspect, the present invention provides a method of expressing a polypeptide, comprising contacting a cell with a nucleic acid molecule comprising a sequence encoding the polypeptide operably linked to a promoter and a repressor sequence, wherein the nucleic acid molecule comprises all or a portion of a viral genome and wherein the cell express a protein that binds to the repressor sequence; and incubating the cell under conditions sufficient to express the polypeptide. In embodiments of this type, the viral genome may be a lentiviral genome (e.g., an HIV). In some aspects, the repressor sequence may be the tetracycline operator sequence and the protein may be the tetracycline repressor protein and conditions sufficient to express the polypeptide comprise incubating the cell in the presence of a compound that reduces binding of the protein to the repressor sequence (e.g., tetracycline).

The present invention also relates to kits for carrying out methods of the invention, and particularly for use in creating recombinant viral vectors and/or nucleic acids molecules of the invention. Kits of the invention may also comprise further components for further manipulating nucleic acids and/or viral vectors produced by methods of the invention. Kits of the invention may comprise one or more nucleic acid molecules comprising all or a portion of a viral genome. Such kits may optionally comprise one or more additional components selected from the group consisting of one or more host cells (e.g., two, three, four, five etc.), one or more reagents for introducing (e.g., by transfection or transformation) molecules or compounds into one or more host cells, one or more nucleotides, one or more polymerases and/or reverse transcriptases (e.g., two, three, four, five, etc.), one or more suitable buffers (e.g., two, three, four, five, etc.), one or more primers (e.g., two, three, four, five, seven, ten, twelve, fifteen, twenty, thirty, fifty, etc.), one or more populations of molecules for creating combinatorial libraries (e.g., two, three, four, five, seven, ten, twelve, fifteen, twenty, thirty, fifty, etc.) and one or more combinatorial libraries (e.g., two, three, four, five, seven, ten, twelve, fifteen, twenty, thirty, fifty, etc.). Kits of the invention may also contain directions or protocols for carrying out one or more methods of the invention.

In another aspect the invention provides kits for joining, deleting, or replacing nucleic acid segments in the viral vectors and/or nucleic acids molecules of the invention, these kits comprising at least one component selected from the group consisting of (1) one or more recombination proteins; (2) one or more compositions comprising one or more recombination proteins; (3) at least one nucleic acid molecule comprising one or more recombination sites (preferably a vector having at least two different recombination specificities); (4) one or more nucleic acid molecules comprising all or a portion of a viral genome and one or more recombination sites; (5) one or more enzymes having ligase activity; (6) one or more enzymes having polymerase activity; (7) one or more enzymes having reverse transcriptase activity; (9) one or more enzymes having restriction endonuclease activity; (10) one or more primers; (11) one or more nucleic acid libraries; (12) one or more reagents for introducing macromolecules into cells; (13) one or more buffers; (14) one or more detergents or solutions containing detergents; (15) one or more nucleotides; (16) one or more terminating agents; (17) one or more transfection reagents; (18) one or more host cells; (19) one or more topoisomerases; (20) one or more nucleic acid molecules to which at least one topoisomerases is bound; (21) one or more nucleic acid molecules comprising at least one topoisomerases recognition sequence; and (22) instructions for using kit components.

Further, kits of the invention may contain one or more recombination proteins. Any recombination protein known to those skilled in the art may be provided in the kits of the invention. Examples of suitable recombination proteins include, but are not limited to, Cre, Int, IHF, Xis, Flp, Fis, Hin, Gin, Cin, Tn3 resolvase, ΦC31, TndX, XerC, and XerD.

In addition, kits of the invention may contain one or more nucleic acids having more than one recombination site (e.g., one or more recombination sites with different recombination specificities such as att sites with different seven base pair overlap regions). In specific embodiments, kits of the invention contain compositions comprising one or more recombination proteins capable of catalyzing recombination between recombination sites, e.g., between att sites. In related embodiments, these compositions comprise one or more recombination proteins capable of catalyzing attB×attP (BP) reactions, attL×attR (LR) reactions, or both BP and LR reactions.

The invention also relates to compositions for carrying out methods of the invention and to compositions created while carrying out methods of the invention. In particular, the invention includes recombinant viral vectors prepared by methods of the invention, methods for preparing host cells that contain these viral vectors, host cells prepared by these methods, and methods employing these host cells for producing products (e.g., RNA, protein, etc.) encoded by these viral vectors, and products encoded by these viral vectors (e.g., RNA, protein, etc.).

Compositions, methods and kits of the invention may be prepared and carried out using a phage-lambda site-specific recombination system, such as with the GATEWAY™ Recombinational Cloning System available from Invitrogen Corporation, Carlsbad, Calif. The GATEWAY™ Technology Instruction Manual (catalog number 12539-011, version C, Invitrogen Corporation, Carlsbad, Calif.) describes in more detail this system and is incorporated herein by reference in its entirety.

Other embodiments of the invention will be apparent to one or ordinary skill in the art in light of what is known in the art, in light of the following drawings and description of the invention, and in light of the claims.

### BRIEF DESCRIPTION OF THE FIGURES

FIG. 1 is a schematic representation of the basic recombinational cloning reaction.

FIG. 2 is a schematic representation of the use of the present invention to clone two nucleic acid segments by performing an LR recombination reaction.

FIGS. 3A to 3D illustrate various embodiments of compositions and methods of the invention for generating a covalently linked double-stranded recombinant nucleic acid molecule. Topoisomerase is shown as a solid circle, and is either attached to a terminus of a substrate nucleic acid molecule or is released following a linking reaction. As illustrated, the substrate nucleic acid molecules have 5′ overhangs, although they similarly can have 3′ overhangs or can be blunt ended. In addition, while the illustrated nucleic acid molecules are shown having the topoisomerases bound thereto (topoisomerase-charged), one or more of the termini shown as having a topoisomerase bound thereto also can be represented as having a topoisomerase recognition site, in which case the joining reaction would further require addition of one or more site specific topoisomerases, as appropriate.

FIG. 3A shows a first nucleic acid molecule having a topoisomerase linked to each of the 5′ terminus and 3′ terminus of one end, and further shows linkage of the first nucleic acid molecule to a second nucleic acid molecule.

FIG. 3B shows a first nucleic acid molecule having a topoisomerase bound to the 3′ terminus of one end, and a second nucleic acid molecule having a topoisomerase bound to the 3′ terminus of one end, and further shows a covalently linked double-stranded recombinant nucleic acid molecule generated due to contacting the ends containing the topoisomerase-charged substrate nucleic acid molecules.

FIG. 3C shows a first nucleic acid molecule having a topoisomerase bound to the 5′ terminus of one end, and a second nucleic acid molecule having a topoisomerase bound to the 5′ terminus of one end, and further shows a covalently linked double-stranded recombinant nucleic acid molecule generated due to contacting the ends containing the topoisomerase-charged substrate nucleic acid molecules.

FIG. 3D shows a nucleic acid molecule having a topoisomerase linked to each of the 5′ terminus and 3′ terminus of both ends, and further shows linkage of the topoisomerase-charged nucleic acid molecule to two nucleic acid molecules, one at each end. The topoisomerases at each of the 5′ termini and/or at each of the 3′ termini can be the same or different.

FIG. 4 is a schematic representation of one embodiment of the invention.

FIGS. 5A-5F are schematic representation of exemplary vectors of the invention. FIG. 5A depicts a vector that contains two different DNA inserts, the transcription of which is driven in different directions by promoters (e.g., polyhedrin, p10, T7, CMV, MMTV, metalothionine, RSV, SV40, hGH promoters). Depending on the type of transcripts which are to be produced, either of DNA-A and/or DNA-B may be in an orientation which results in the production of either sense or anti-sense RNA.

FIG. 5B is a schematic representation of an exemplary vector of the invention which contains one DNA insert, the transcription of which may proceed in either direction (or both directions) driven by two promoters which may be the same or different. Thus, RNA produced by transcription driven by one promoter will be sense RNA and RNA produced by transcription driven by the other promoter will be anti-sense RNA. RNA can be produced from both promoters, for example, to make small interfering RNA (siRNA).

FIG. 5C is a schematic representation of an exemplary vector of the invention which contains two different DNA inserts having the same nucleotide sequence (i.e., DNA-A), the transcription of which are driven in different directions by two separate promoters, which may be the same or different. In this example, RNA produced by transcription driven by one promoter will be sense RNA and RNA produced by transcription driven by the other promoter will be anti-sense RNA.

FIG. 5D is a schematic representation of an exemplary vector of the invention that contains two DNA inserts having the same nucleotide sequence (i.e., DNA-A) in opposite orientations, the transcription of which is driven by one promoter (e.g., CMV promoter). A transcription termination signal is not present between the two copies of DNA-A and the DNA-A inserts. Transcription of one segment produces a sense RNA and of the other produces an anti-sense RNA. The RNA produced from this vector will undergo intramolecular hybridization and, thus, will form a double-stranded molecule with a hairpin turn.

FIGS. 5E and 5F are schematic representations of two exemplary vectors of the invention, each of which contains a DNA insert having the same nucleotide sequence (i.e., DNA-A). Transcription of these inserts results in the production of sense and anti-sense RNA which may then hybridize to form double stranded RNA molecules.

FIG. 6 is a plasmid map of pAd/CMV/V5-DEST.

FIG. 7 is a plasmid map of pAd-GW-TO/tRNA.

FIG. 8 is a plasmid map of pAdenoTAG tRNA.

FIG. 9 is a plasmid map of pAd/PL-DEST.

FIG. 10 is a plasmid map of pAd/CMV/V5-GW/lacZ.

FIG. 11 shows the recombination region of pAd/CMV/V5-DEST.

FIG. 12 shows the recombination region of pAd/PL-DEST.

FIG. 13 shows a schematic representation of producing an exemplary adenoviral vector produced as described in Example 4.

FIGS. 14A-C show the cytopathic effect (CPE) in 293A cells transfected with Pac I-digested pAd/CMV/V5-GW/lacZ plasmid as described in Example 4. FIG. 14A shows 293A cells at days 4-6 post-transfection. At this early stage, cells producing adenovirus first appear as patches of rounding, dying cells. FIG. 14B shows 293A cells at day 6-8 post-transfection. As the infection proceeds, cells containing viral particles lyse and infect neighboring cells. A plaque begins to form. FIG. 14C shows cells at day 8-10 post-transfection At this late stage, infected neighboring cells lyse, forming a plaque that is clearly visible.

FIG. 15 is a plasmid map of pIB/V5-His-DEST.

FIG. 16 provides the nucleotide sequence of the OpIE2 promoter.

FIG. 17 shows the recombination region of pIB/V5-His-DEST.

FIG. 18 is a plasmid map of pIB/V5-His-GW/lacZ.

FIG. 19A shows a schematic representation of the BaculoDirect™ V5-His Dest cassette. FIG. 19B shows a schematic representation of the BaculoDirect™ Mel/V5-His Dest cassette.

FIG. 20 shows a schematic representation of the genome of a baculovirus of the invention and an entry clone to introduce a gene of interest into the baculoviral genome.

FIG. 21 shows a schematic representation of the topoisomerase mediate insertion of the gp64 promoter into pIB/V5-His.

FIG. 22 is a plasmid map of pIB/V5-His/gp64/DEST.

FIG. 23 is a bar graph showing the results of a transient transfection assay.

FIG. 24 is a Western blot showing protein expression levels of stably transfected cells and transiently transfected cells.

FIGS. 25A and 25B are Western blots showing protein expression levels of stably transfected cells.

FIG. 26 is a bar graph showing the results of a lacZ transfection assay.

FIG. 27A shows a schematic representation of the construction of BaculoDirect™ vector. FIG. 27B shows a schematic representation of an LR reaction between the BaculoDirect™ vector and an entry clone containing a gene of interest.

FIG. 28 shows a schematic representation of a high throughput cloning protocol using the baculoviruses of the present invention.

FIG. 29 shows the results of a comparison of the use of circular virus DNA and linear virus DNA in the initial LR clonase reaction.

FIG. 30 shows the results obtained in the presence of ganciclovir selection.

FIG. 31 shows the results of a Western blot of various polypeptides expressed using BaculoDirect™.

FIG. 32 shows a comparison of the titers of recombinant baculoviruses obtained using various techniques. Virus titer was obtained using the TCID₅₀ technique (upper panel) and by plaque assay (lower panel).

FIG. 33 shows a comparison of the cumulative time required to prepare a viral stock using Bac to Bac™ and BaculoDirect™.

FIG. 34 shows a schematic representation of plasmid pVL1393 GST p10 stop.

FIG. 35 shows a schematic representation of a method of making a nucleic acid molecule comprising all or a portion of a lentiviral genome.

FIG. 36 shows a schematic representation of plasmids for use in the present invention. FIG. 36A shows a schematic representation pLenti6/V5-DEST. FIG. 36B shows a schematic representation of pLenti6/V5-D-TOPO®. FIG. 36C shows a plasmid map of pLenti4/V5-DEST. FIG. 36D shows a plasmid map of pLenti6/UbC/V5-DEST.

FIG. 37 shows a schematic representation of plasmids for use in the present invention. FIG. 37A shows a schematic representation pLP1. FIG. 37B shows a schematic representation of pLP2. FIG. 37C shows a schematic representation of pLP/VSVG.

FIG. 38 shows the results of an experiment in which two LR reactions were performed with either pLenti6/V5-DEST alone or pLenti6/V5-DEST plus pENTR/CAT and 3 μl of each was transformed into TOP10 cells. 100 μl of the transformations were plated on regular LB-amp plates (no Bsd) or LB-amp containing 50 μg/ml blasticidin. FIG. 38A is photograph shown the observed colony morphologies. FIG. 38B shows the results in tabular form.

FIGS. 39A and 39B show the results of a Western blot with anti-lacZ antibody (FIG. 39A) and anti-V5-antibody (FIG. 39B).

FIG. 40 shows in tabular form the titers of lentiviral stocks prepared with inserts of varying size.

FIGS. 41A, 41B, and 41C show the expression of marker genes using the lentiviral expression system. FIG. 41A shows the expression of lacZ using the GATEWAY™ adapted lentiviral system. FIGS. 41B and 41C show the expression of GFP using the topoisomerase adapted lentiviral system.

FIGS. 42A and 42B show Western blots of the expression of various genes using the lentiviral expression system described herein. FIG. 42A shows the expression of lacZ, CAT and GFP. FIG. 42B shows the expression o PKC and GFP.

FIGS. 43A and 43B show the results of varying the multiplicity of infection on the observed expression level of lacZ using the lentiviral expression system of the invention. FIG. 43A shows photographs cells stained to detect β-galactosidase activity. FIG. 43B is a graph of β-galactosidase activity as a function of MOI.

FIGS. 44A and 44B show the results of transduction of various cell types with lentiviral vectors prepared according to the methods of the invention. FIG. 44A is a bar graph of β-galactosidase activity observed in various actively growing or G1/S arrested cell types. FIG. 44B provides photographs of contacted-inhibited primary foreskin cells transduced with lentiviral vectors and stained to detect lacZ activity.

FIGS. 45A and 45B show long term expression of genes from cells transduced with the nucleic acid molecules of the invention. FIG. 45A shows photographs of transduced cells stained for β-galactosidase activity after 10 days. FIG. 45B shows photographs of transduced cells stained for β-galactosidase activity after 6 weeks.

FIG. 46A shows the recombination region of pLenti6/V5-DEST.

FIG. 46B shows the recombination region of the expression clone resulting from pLenti6/UbC/V5-DEST×entry clone. FIG. 46C shows the complete sequence of the UbC promoter.

FIG. 47 is a schematic representation of directional topoisomerase cloning according to the invention.

FIG. 48 shows the cloning region of pLenti6/V5-D-TOPO®.

FIG. 49 shows a plasmid map of pCMVSPORT6TAg.neo.

FIG. 50 shows a schematic representation of the Tag-On-Demand™ method described in Example 14. A coding sequence of interest (GOI) is cloned with a TAG stop codon into an expression vector such that it is operably linked to a promoter (as an example, the CMV promoter is indicated in the figure). If its native stop codon is not TAG, it must be changed to TAG to be compatible with this particular method although by changing the anticodon on the suppressor tRNA molecule any stop codon can be used. Downstream of, and in frame with, the GOI is an epitope tag to be fused to the C-terminus of the protein of interest (e.g., V5, GFP, etc.). Under normal expression conditions (i.e., in the absence of tRNA suppressor (−tRNA^{TAG})), native protein is expressed. In the presence of the tRNA suppressor (+tRNA), the TAG stop codon is translated as a serine in this example, and translation continues along to produce a tagged protein. The expression vector contains at least one non-TAG stop codon (e.g., TAA or TGA) downstream of the C-terminal epitope tag to terminate translation of the fusion protein.

FIGS. 51A-B shows western blots from plasmid tRNA suppression using the V5 epitope and GFP Tag-On-Demand™ method described in Example 14. FIG. 51A shows the western blots of CHO cells that were co-transfected with one of three reporters: pcDNA3.2/V5-GW/CAT^{TAA}, -GW/CAT^{TAG} or -GW/CAT^{TGA} in the presence or absence of its cognate tRNA suppressor: pUC12-tRNA^{TAA}, pUC12-tRNA^{TAG} or pUC12-tRNA^{TAA}, as indicated. Forty-eight hours post transfection, 20 μg of cell lysate was analyzed by either anti-V5 or anti-CAT western blotting as indicated. A control transfection of pcDNA3.1/CAT was also included in each experiment (CAT lane). FIG. 5 B is the western blot of 293FT cells that were co-transfected with one of three reporters: pcDNA6.2/GFP-GW/CAT^{TAA}, -GW/CAT^{TAG} or -GW/CAT^{TGA} and one of the tRNA suppressors: pUCl2-tRNA ^{TAA}, pUC12-tRNA^{TAG} or pUC12-tRNA^{TGA}, as indicated. Forty-eight hours post transfection, 20 μg of cell lysate was analyzed by anti-CAT western blotting as indicated. A control transfection of pcDNA3.1/CAT was also included in each experiment (CAT lane).

FIG. 52 shows the stop codon specificity of tRNA suppression using plasmid tRNA suppression. CHO cells were co-transfected with pcDNA3.1/lacZ-stop^{TAG}-GFP and one of each of the three tRNA suppressors: pUC12-tRNA^{TAA}, pUC12-tRNA ^{TGA} and pUC12-tRNA^{TGA}. Forty-eight hours post-transfection, brightfield (upper panes) and fluorescent (lower panels) photographs were taken.

FIG. 53 shows the expression of the gene of interest after adenovirus delivery of the monomer vs. octamer tRNA^{TAG} construct. COS-7 cells were transduced with crude lysates of Adeno-tRNA^{TAG} (monomer) or Adeno-tRNA8^{TAG} (octamer) at an MOI of 50 for 6 hours, followed by an overnight transfection with pcDNA3.1/lacZ-stop^{TAG}-GFP. 72 hours post-transduction, fluorescent photographs (upper panels) and anti-lacZ western blotting (lower panel) were performed. Lane 1: mock, Lane 2: co-transfection of pUC 12-tRNA^{TAG} and reporter vector (positive control), Lane 3: Adeno-tRNA^{TAG} (monomer), Lane 4: Adeno-tRNA8^{TAG} (octamer).

FIG. 54 shows the expression of the indicated pENTR-ORF clone.

Three pENTR-ORF clones were taken from the Invitrogen Corporation, Carlsbad, Calif. human ORF collection and L×R crossed into either pcDNA6.2/GFP-DEST or pcDNA6.2/V5-DEST to create expression vectors. COS-7 cells were transduced with Ad-tRNA8^{TAG} (MOI 50) followed by transfection with the ORF expression vectors. Twenty-four hours post transfection, fluorescent photographs were taken (upper panels). V5-western blotting was performed on RIPA lysates following co-transfection of COS-7 cells with the ORF expression clone and the pUC12-tRNA^{TAG} (lower panel). ORF6 expresses a protein similar to CGI-130, ORF7 expresses a splicing factor and ORF12 expresses a truncated c-myc p64 protein. “lacZ” refers to pcDNA3.1/lacZ-stop^{TAG}-V5 and “GFP-V5” refers to constitutive GFP expression from pcDNA5/GFP.

FIGS. 55A and 55B shows western blots from cells transduced with adenovirus-tRNA^{TAG} for the suppression of either transient or stable target genes. FIG. 55A shows a western blot of the tRNA suppression of a stably-expressed target gene. FlpIn-CHO cells stably expressing a single copy of pcDNA6/FRT/lacZ-stop-^{TAG}-GFP were transduced with Adeno-tRNA8^{TAG} at various MOIs. 48 hours post-transduction, cell lysates were analyzed by anti-lacZ western blotting and percent suppression was determined by densitometry. The additional band present in the “stable GOI” western blot (indicated by *) is the endogenous lacZeo fusion protein present in the Flp-In CHO cell line. FIG. 55B shows a western blot of the tRNA suppression of a transiently-expressed target gene. COS-7 cells were transiently transfected with the plasmid pcDNA3.1 /lacZ-stop^{TAG}-GFP following transduction with CsCl purified Adeno-tRNA8^{TAG} at various MOIs. 48 hours post-transduction, cell lysates were analyzed by anti-lacZ western blotting and percent suppression was determined by densitometry.

FIG. 56 shows the use of the Tag-On-Demand™ method in five mammalian cell lines. BHK-21, CHO—S, COS-7, HeLa and HT1080 cells were transduced with CsCl purified Adeno-tRNA8^{TAG} at an MOI of 50 followed by a transfection with pcDNA3.1/lacZ-stop^{TAG}-GFP. Brightfield (upper panels) and fluorescent (lower panels) photographs were taken 48 hours post transduction.

FIG. 57 is a plasmid map of pcDNA™6.2/V5-DEST.

FIG. 58 is a plasmid map of pcDNA™6.2/GFP-DEST.

FIG. 59 is a plasmid map of pcDNA™6.2/V5-GW/p64^{TAG}.

FIG. 60 is a plasmid map of pcDNA™6.2/GFP-GW/p64^{TAG}.

FIGS. 61A and 61B provide the sequences of the recombination regions of vectors pcDNA™6.2/V5-DEST and pcDNA™6.2/GFP-DEST, respectively.

FIG. 62 provides a schematic representation of a method of using an adenovirus of the invention to produce C-terminal fusion proteins in a transient transfection experiment.

FIG. 63 provides a schematic representation of a method of using an adenovirus of the invention to produce C-terminal fusion proteins in a stable cell line containing an expression construct.

FIG. 64 shows fluorescent micrographs of GFP-fusion proteins made using the present invention.

FIG. 65 shows a schematic of the use of a fluorogenic substrate to assay β-lactamase activity according to one aspect of the invention.

FIG. 66 shows a comparison of sequential (left column) versus simultaneous (right column) transduction/transfection.

FIG. 67 shows Western blots showing the effects of various lipid/DNA ratios and MOI in a simultaneous transduction/transfection method (upper panels) and a sequential transduction/transfection method (lower panels).

FIG. 68 is a Western blot showing the results of an experiment in which COS-7 cells were transduced with an adenovirus expressing suppressor tRNA molecules at various MOIs and simultaneously transfected with the pcDNA™6.2/GFP-GW/p64^{TAG} plasmid.

FIG. 69 is a vector map of pLenti6/TR, a nucleic acid molecule of the invention that can be used to generate blasticidin resistant mammalian cells that stably express the tetracycline repressor, TetR.

FIG. 70 is a vector map of pLenti4/TO/V5-DEST, a nucleic acid molecule of the invention.

FIG. 71 is a vector map of pLenti6/V5.

FIG. 72 is a vector map of pLenti3/V5-TREx.

FIG. 73 shows a schematic representation of a method of attaching a topoisomerase to a nucleic acid molecule of the invention.

### DETAILED DESCRIPTION OF THE INVENTION

Definitions

In the description that follows, a number of terms used in recombinant nucleic acid technology are utilized extensively. In order to provide a clear and more consistent understanding of the specification and claims, including the scope to be given such terms, the following definitions are provided.

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Structural Gene: As used herein, the phrase “structural gene” refers to refers to a nucleic acid that is transcribed into messenger RNA that is then translated into a sequence of amino acids characteristic of a specific polypeptide.

Host: As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, operators (e.g., the tet operator), and the like.

Promoter: As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Target Nucleic Acid Molecule: As used herein, the phrase “target nucleic acid molecule” refers to a nucleic acid segment of interest, preferably nucleic acid that is to be acted upon using the compounds and methods of the present invention. Such target nucleic acid molecules may contain one or more (e.g., two, three, four, five, seven, ten, twelve, fifteen, twenty, thirty, fifty, etc.) genes or one or more portions of genes.

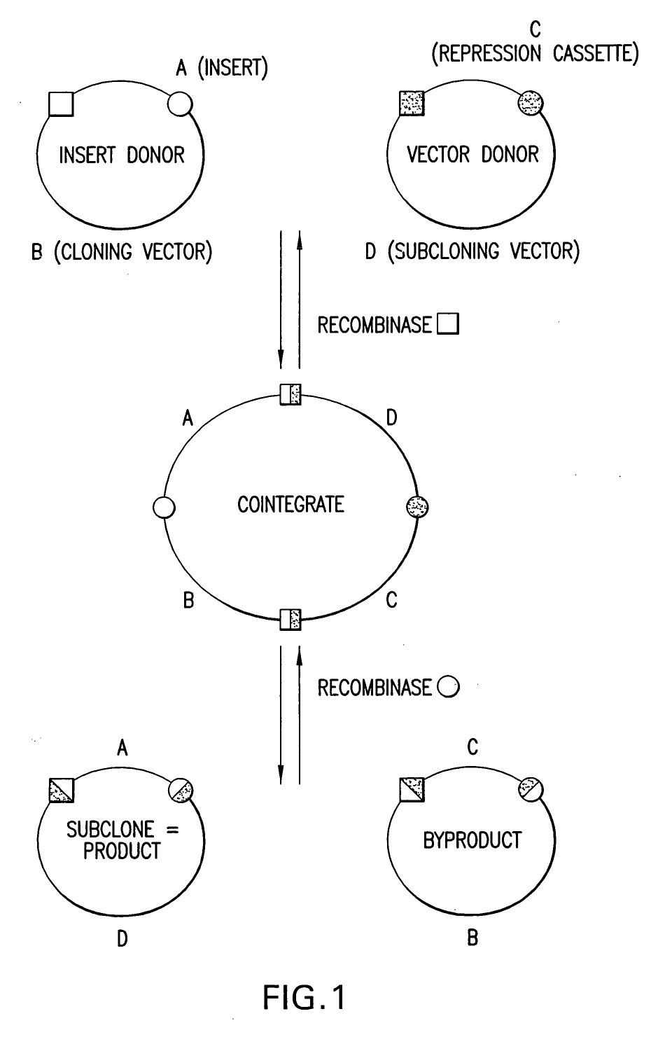

Insert Donor: As used herein, the phrase “Insert Donor” refers to one of the two parental nucleic acid molecules (e.g., RNA or DNA) of the present invention that carries the an insert (see FIG. 1). The Insert Donor molecule comprises the insert flanked on both sides with recombination sites. The Insert Donor can be linear or circular. In one embodiment of the invention, the Insert Donor is a circular nucleic acid molecule, optionally supercoiled, and further comprises a cloning vector sequence outside of the recombination signals. When a population of inserts or population of nucleic acid segments are used to make the Insert Donor, a population of Insert Donors result and may be used in accordance with the invention. An Insert Donor may be referred to as an Entry Clone.

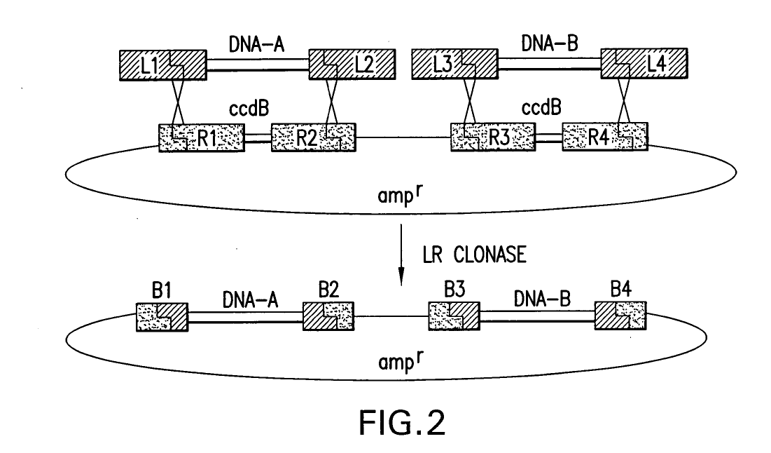

Insert: As used herein, the term “insert” refers to a desired nucleic acid segment that is a part of a larger nucleic acid molecule. In many instances, the insert will be introduced into the larger nucleic acid molecule. For example, the nucleic acid segments labeled ccdB, DNA-A, and DNA-B in FIG. 2, are nucleic acid inserts with respect to the larger nucleic acid molecule shown therein. In most instances, the insert will be flanked by recombination sites, topoisomerase sites and/or other recognition sequences (e.g. at least one recognition sequence will be located at each end). In certain embodiments, however, the insert will only contain a recognition sequence on one end.

Product: As used herein, the term “Product” refers to one the desired daughter molecules comprising the A and D sequences that is produced after the second recombination event during the recombinational cloning process (see FIG. 1). The Product contains the nucleic acid that was to be cloned or subcloned. In accordance with the invention, when a population of Insert Donors are used, the resulting population of Product molecules will contain all or a portion of the population of Inserts of the Insert Donors and preferably will contain a representative population of the original molecules of the Insert Donors.

Byproduct: As used herein, the term “Byproduct” refers to a daughter molecule (a new clone produced after the second recombination event during the recombinational cloning process) lacking the segment that is desired to be cloned or subcloned.

Cointegrate: As used herein, the term “Cointegrate” refers to at least one recombination intermediate nucleic acid molecule of the present invention that contains both parental (starting) molecules. Cointegrates may be linear or circular. RNA and polypeptides may be expressed from cointegrates using an appropriate host cell strain, for example E. coli DB3.1 (particularly E. coli LIBRARY EFFICIENCY® DB3.1™ Competent Cells), and selecting for both selection markers found on the cointegrate molecule.

Recognition Sequence: As used herein, the phrase “recognition sequence” or “recognition site” refers to a particular sequence to which a protein, chemical compound, DNA, or RNA molecule (e.g., restriction endonuclease, a modification methylase, topoisomerases, or a recombinase) recognizes and binds. In the present invention, a recognition sequence may refer to a recombination site or topoisomerases site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, SpCCE1, and ParA.

Recombinases: As used herein, the term “recombinases” is used to refer to the protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

Recombination Site: A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994)). Other examples of recombination sites include the attB, attP, attL, and attR sequences described in U.S. provisional patent applications 60/136,744, filed May 28, 1999, and 60/188,000, filed Mar. 9, 2000, and in co-pending U.S. patent applications Ser. Nos. 09/517,466 and 09/732,91—all of which are specifically incorporated herein by reference-and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site flanked by recombination sites.

Topoisomerase recognition site. As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372 also incorporated herein by reference). In comparison, the nucleotide sequence 5′-GCAACTT-3′ is the topoisomerase recognition site for type IA E. coli topoisomerase III.

Recombinational Cloning: As used herein, the phrase “recombinational cloning” refers to a method, such as that described in U.S. Pat. Nos. 5,888,732; 6,143,557; 6,171,861; 6,270,969; and 6,277,608 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466 filed Mar. 2, 2000, and in published U.S. application No. 2002 0007051-A1, all assigned to the Invitrogen Corporation, Carlsbad, Calif., the disclosures of which are specifically incorporated herein in their entirety. In brief, the GATEWAY™ Cloning System described in these patents and applications utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in GATEWAY™, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

Repression Cassette: As used herein, the phrase “repression cassette” refers to a nucleic acid segment that contains a repressor or a selectable marker present in the subcloning vector.

Selectable Marker: As used herein, the phrase “selectable marker” refers to a nucleic acid segment that allows one to select for or against a molecule (e.g., a replicon) or a cell that contains it and/or permits identification of a cell or organism that contains or does not contain the nucleic acid segment. Frequently, selection and/or identification occur under particular conditions and do not occur under other conditions.

Markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as β-lactamase, β-galactosidase, green fluorescent protein (GFP), yellow flourescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; and/or (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Selection and/or identification may be accomplished using techniques well known in the art. For example, a selectable marker may confer resistance to an otherwise toxic compound and selection may be accomplished by contacting a population of host cells with the toxic compound under conditions in which only those host cells containing the selectable marker are viable. In another example, a selectable marker may confer sensitivity to an otherwise benign compound and selection may be accomplished by contacting a population of host cells with the benign compound under conditions in which only those host cells that do not contain the selectable marker are viable. A selectable marker may make it possible to identify host cells containing or not containing the marker by selection of appropriate conditions. In one aspect, a selectable marker may enable visual screening of host cells to determine the presence or absence of the marker. For example, a selectable marker may alter the color and/or fluorescence characteristics of a cell containing it. This alteration may occur in the presence of one or more compounds, for example, as a result of an interaction between a polypeptide encoded by the selectable marker and the compound (e.g., an enzymatic reaction using the compound as a substrate). Such alterations in visual characteristics can be used to physically separate the cells containing the selectable marker from those not contain it by, for example, fluorescent activated cell sorting (FACS).

Multiple selectable markers may be simultaneously used to distinguish various populations of cells. For example, a nucleic acid molecule of the invention may have multiple selectable markers, one or more of which may be removed from the nucleic acid molecule by a suitable reaction (e.g., a recombination reaction). After the reaction, the nucleic acid molecules may be introduced into a host cell population and those host cells comprising nucleic acid molecules having all of the selectable markers may be distinguished from host cells comprising nucleic acid molecules in which one or more selectable markers have been removed (e.g., by the recombination reaction). For example, a nucleic acid molecule of the invention may have a blasticidin resistance marker outside a pair of recombination sites and a β-lactamase encoding selectable marker inside the recombination sites. After a recombination reaction and introduction of the reaction mixture into a cell population, cells comprising any nucleic acid molecule can be selected for by contacting the cell population with blasticidin. Those cell comprising a nucleic acid molecule that has undergone a recombination reaction can be distinguished from those containing an unreacted nucleic acid molecules by contacting the cell population with a fluorogenic β-lactamase substrate as described below and observing the fluorescence of the cell population. Optionally, the desired cells can be physically separated from undesirable cells, for example, by FACS.

In a specific embodiment of the invention, a selectable marker may be a nucleic acid sequence encoding a polypeptide having an enzymatic activity (e.g., β-lactamase activity). Assays for β-lactamase activity are known in the art. U.S. Pat. No. 5,955,604, issued to Tsien, et al. Sep. 21, 1999, U.S. Pat. No. 5,741,657 issued to Tsien, et al., Apr. 21, 1998, 6,031,094, issued to Tsien, et al., Feb. 29, 2000, U.S. Pat. No. 6,291,162, issued to Tsien, et al., Sep. 18, 2001, and U.S. Pat. No. 6,472,205, issued to Tsien, et al. Oct. 29, 2002, disclose the use of β-lactamase as a reporter gene and fluorogenic substrates for use in detecting β-lactamase activity and are specifically incorporated herein by reference. In one embodiment of the invention, a selectable marker may be a nucleic acid sequence encoding a polypeptide having β-lactamase activity and desired host cells may be identified by assaying the host cells for β-lactamase activity.

A β-lactamase catalyzes the hydrolysis of a β-lactam ring. Those skilled in the art will appreciate that the sequences of a number of polypeptides having β-lactamase activity are known. In addition to the specific β-lactamases disclosed in the Tsien, et al. patents listed above, any polypeptide having β-lactamase activity is suitable for use in the present invention.

β-lactamases are classified based on amino acid and nucleotide sequence (Ambler, R. P., Phil. Trans. R. Soc. Lond. [Ser.B.] 289: 321-331 (1980)) into classes A-D. Class A β-lactamases possess a serine in the active site and have an approximate weight of 29 kD. This class contains the plasmid-mediated TEM β-lactamases such as the RTEM enzyme of pBR322. Class B β-lactamases have an active-site zinc bound to a cysteine residue. Class C enzymes have an active site serine and a molecular weight of approximately 39 kD, but have no amino acid homology to the class A enzymes. Class D enzymes also contain an active site serine. Representative examples of each class are provided below with the accession number at which the sequence of the enzyme may be obtained in the indicated database.

Class A β-lactamases

| Bacteroides fragilis CS30 | L13472 | GenBank |
|---|---|---|
| Bacteroides uniformis WAL-7088 | P30898 | SWISS-PROT |
| PER-1, P. aeruginosa RNL-1 | P37321 | SWISS-PROT |
| Bacteroides vulgatus CLA341 | P30899 | SWISS-PROT |
| OHIO-1, Enterobacter cloacae | P18251 | SWISS-PROT |
| SHV-1, K. pneumoniae | P23982 | SWISS-PROT |
| LEN-1, K. pneumoniae LEN-1 | P05192 | SWISS-PROT |
| TEM-1, E. coli | POO810 | SWISS-PROT |
| Proteus mirabilis GN179 | P30897 | SWISS-PROT |
| PSE-4, P. aeruginosa Dalgleish | P16897 | SWISS-PROT |
| Rhodopseudomonas capsulatus SP108 | P14171 | SWISS-PROT |
| NMC, E. cloacae NOR-1 | P52663 | SWISS-PROT |
| Sme-1, Serratia marcescens S6 | P52682 | SWISS-PROT |
| OXY-2, Klebsiella oxytoca D488 | P23954 | SWISS-PROT |
| K. oxytoca E23004/SL781/SL7811 | P22391 | SWISS-PROT |
| S. typhimurium CAS-5 | X92507 | GenBank |
| MEN-1, E. coli MEN | P28585 | SWISS-PROT |
| Serratia fonticola CUV | P80545 | SWISS-PROT |
| Citrobacter diversus ULA27 | P22390 | SWISS-PROT |
| Proteus vulgaris 5E78-1 | P52664 | SWISS-PROT |
| Burkholderia cepacia 249 | U85041 | GenBank |
| Yersinia enterocolitica serotype O:3/Y-56 | Q01166 | SWISS-PROT |
| M. tuberculosis H37RV | Q10670 | SWISS-PROT |
| S. clavuligerusNRRL 3585 | Z54190 | GenBank |
| III, Bacillus cereus 569/H | P06548 | SWISS-PROT |
| B. licheniformis 749/C | P00808 | SWISS-PROT |
| I, Bacillus mycoides NI10R | P28018 | SWISS-PROT |
| I, B. cereus 569/H/9 | P00809 | SWISS-PROT |
| I, B. cereus 5/B | P10424 | SWISS-PROT |
| B. subtilis 168/6GM | P39824 | SWISS-PROT |
| 2, Streptomyces cacaoi DSM40057 | P14560 | SWISS-PROT |
| Streptomyces badius DSM40139 | P35391 | SWISS-PROT |
| Actinomadura sp. strain R39 | X53650 | GenBank |
| Nocardia lactamdurans LC411 | Q06316 | SWISS-PROT |
| S. cacaoi KCC S0352 | Q03680 | SWISS-PROT |
| ROB-1, H. influenzae F990/LNPB51/ | P33949 | SWISS-PROT |
| serotype A1 | | |
| Streptomyces fradiae DSM40063 | P35392 | SWISS-PROT |
| Streptomyces lavendulae DSM2014 | P35393 | SWISS-PROT |
| Streptomyces albus G | P14559 | SWISS-PROT |
| S. lavendulae KCCS0263 | D12693 | GenBank |
| Streptomyces aureofaciens | P10509 | SWISS-PROT |
| Streptomyces cellulosae KCCS0127 | Q06650 | SWISS-PROT |
| Mycobacterium fortuitum | L25634 | GenBank |
| S. aureus PC1/SK456/NCTC9789 | P00807 | SWISS-PROT |
| BRO-1, Moraxella catarrhalis ATCC | Z54181 | GenBank |
| 53879 | Q59514 | SWISS-PROT |

Class B β-lactamase

| | | |
|---|---|---|
| II, B. cereus 569/H | P04190 | SWISS-PROT |
| II, Bacillus sp. 170 | P10425 | SWISS-PROT |
| II, B. cereus 5/B/6 | P14488 | SWISS-PROT |
| Chryseobacterium meningosepticum | X96858 | GenBank |
| CCUG4310 | | |
| IMP-1, S. marcescens AK9373/TN9106 | P52699 | SWISS-PROT |
| B. fragilis TAL3636/TAL2480 | P25910 | SWISS-PROT |
| Aeromonas hydrophila AE036 | P26918 | SWISS-PROT |
| L1, Xanthomonas maltophilia IID 1275 | P52700 | SWISS-PROT |

Class C β-lactamase

| | | |
|---|---|---|
| Citrobacter freundii OS60/GN346 | P05193 | SWISS-PROT |
| E. coli K-12/MG1655 | P00811 | SWISS-PROT |
| P99, E. cloacae P99/Q908R/MHN1 | P05364 | SWISS-PROT |
| Y. enterocolitica IP97/serotype O:5B | P45460 | SWISS-PROT |
| Morganella morganii SLM01 | Y10283 | GenBank |
| A. sobria 163a | X80277 | GenBank |
| FOX-3, K. oxytoca 1731 | Y11068 | GenBank |
| K. pneumoniae NU2936 | D13304 | GenBank |
| P. aeruginosa PAO1 | P24735 | SWISS-PROT |
| S. marcescens SR50 | P18539 | SWISS-PROT |
| Psychrobacter immobilis A5 | X83586 | GenBank |

Class D β-lactamases

| | | |
|---|---|---|
| OXA-18, Pseudomonas aeruginosa Mus | U85514 | GenBank |
| OXA-9, Klebsiella pneumoniae | P22070 | SWISS-PROT |
| Aeromonas sobria AER 14 | X80276 | GenBank |
| OXA-1, Escherichia coli K10-35 | P13661 | SWISS-PROT |
| OXA-7, E. coli 7181 | P35695 | SWISS-PROT |
| OXA-11, P. aeruginosa ABD | Q06778 | SWISS-PROT |
| OXA-5, P. aeruginosa 76072601 | Q00982 | SWISS-PROT |
| LCR-1, P. aeruginosa 2293E | Q00983 | SWISS-PROT |
| OXA-2, Salmonella typhimurium type 1A | P05191 | SWISS-PROT |

For additional β-lactamases and a more detailed description of substrate specificities, consult Bush et al. (1995) Antimicrob. Agents Chemother. 39:1211-1233. Those skilled in the art will appreciate that the polypeptides having β-lactamase activity disclosed herein may be altered by for example, mutating, deleting, and/or adding one or more amino acids and may still be used in the practice of the invention so long as the polypeptide retains detectable β-lactamase activity. An example of a suitably altered polypeptide having β-lactamase activity is one from which a signal peptide sequence has been deleted and/or altered such that the polypeptide is retained in the cytosol of prokaryotic and/or eukaryotic cells. The amino acid sequence of one such polypeptide is provided in Table 30.

As described in the above-referenced United States patents, host cells to be assayed may be contacted with a fluorogenic substrate for β-lactamase activity. In the presence of β-lactamase, the substrate is cleaved and the fluorescence emission spectrum of the substrate is altered. As an example, un-cleaved substrate may fluoresce green (i.e., have an emission maxima at approximately 520 nm) when excited with light having a wavelength of 405 nm and the cleaved substrate may fluoresce blue (i.e., have an emission maxima at approximately 447 nm). By determining the ratio of green fluorescence intensity to blue fluorescence intensity it is possible to determine the amount of β-lactamase produced and from that, to calculate what % of the cells express β-lactamase. Kits for conducting a fluorescence-based β-lactamase assay are commercially available, for example, from PanVerra, LLC, Madison, Wis., catalog number K1032.

Preferred β-lactam fluorogenic substrates for use in the present invention include those which comprise a fluorescence donor moiety and a fluorescence acceptor moiety linked to a cephalosporin backbone such that, upon hydrolysis of the β-lactam, the acceptor moiety is released from the molecule. Before the β-lactam is hydrolyzed, the donor and acceptor moiety are positioned such that efficient fluorescence resonance energy transfer (FRET) occurs. Upon excitation with light of a suitable wavelength, fluorescence from the acceptor moiety is observed. After hydrolysis of the β-lactam, the acceptor moiety is released from the molecule and the FRET is disrupted resulting in a change in the fluorescence emission spectrum. An example of a suitable fluorescence donor molecule is a coumarin or derivative thereof (e.g., 6-chloro-7-hydroxycoumarin) and examples of suitable acceptor moieties include, but are not limited to, fluorescein, rhodol, or rhodamine or derivatives thereof. Examples of suitable substrates include CCF2 and the acetoxymethyl ester derivative thereof (CCF2/AM). Those skilled in the art will appreciate that CCF2/AM is membrane permeable and is converted to CCF2 inside a cell by the action of endogenous esterase enzymes. A schematic showing the result of hydrolysis of CCF2 by a β-lactamase is shown in FIG. 65.

Selection Scheme: As used herein, the phrase “selection scheme” refers to any method that allows selection, enrichment, or identification of a desired nucleic acid molecules or host cells containing them (in particular Product or Product(s) from a mixture containing an Entry Clone or Vector, a Destination Vector, a Donor Vector, an Expression Clone or Vector, any intermediates (e.g., a Cointegrate or a replicon), and/or Byproducts). In one aspect, selection schemes of the invention rely on one or more selectable markers. The selection schemes of one embodiment have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some preferred embodiments, the selection scheme results in selection of, or enrichment for, only one or more desired nucleic acid molecules (such as Products). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one embodiment, the selection schemes (which can be carried out in reverse) will take one of three forms, which will be discussed in terms of FIG. 1. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait.”)

Examples of such toxic gene products are well known in the art, and include, but are not limited to, restriction endonucleases (e.g., DpnI, Nla3, etc.); apoptosis-related genes (e.g., ASK1 or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from ΦX174 or bacteriophage T4; antibiotic sensitivity genes such as rpsL; antimicrobial sensitivity genes such as pheS; plasmid killer genes' eukaryotic transcriptional vector genes that produce a gene product toxic to bacteria, such as GATA-1; genes that kill hosts in the absence of a suppressing function, e.g., kicB, ccdB, ΦX174 E (Liu, Q., et al., Curr. Biol. 8:1300-1309 (1998)); and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention (see, e.g., U.S. Pat. No. 4,960,707 (DpnI and DpnII); U.S. Pat. Nos. 5,082,784 and 5,192,675 (KpnI); U.S. Pat. No. 5,147,800 (NgoAIII and NgoAI); U.S. Pat. No. 5,179,015 (FspI and HaeIII): U.S. Pat. No. 5,200,333 (HaeII and TaqI); U.S. Pat. No. 5,248,605 (HpaII); U.S. Pat. No. 5,312,746 (ClaI); U.S. Pat. Nos. 5,231,021 and 5,304,480 (XhoI and XhoII); U.S. Pat. No. 5,334,526 (AluI); U.S. Pat. No. 5,470,740 (NsiI); U.S. Pat. No. 5,534,428 (SstI/SacI); U.S. Pat. No. 5,202,248 (NcoI); U.S. Pat. No. 5,139,942 (NdeI); and U.S. Pat. No. 5,098,839 (Pacd). (See also Wilson, G. G., Nucl. Acids Res. 19:2539-2566 (1991); and Lunnen, K. D., et al., Gene 74:25-32 (1988)).

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-Specific Recombinase: As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)). Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Suppressor tRNAs. A tRNA molecule that results in the incorporation of an amino acid in a polypeptide in a position corresponding to a stop codon in the mRNA being translated.

Homologous Recombination: As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination that occurs, for example, at recombination sites such as att sites, is not considered to be “homologous recombination,” as the phrase is used herein.

Vector: As used herein, the term “vector” refers to a nucleic acid molecule (preferably DNA) that provides a useful biological or biochemical property to an insert. A vector may be a nucleic acid molecule comprising all or a portion of a viral genome. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule that includes, preferably, an appropriate replicon. In the present invention, the subcloning vector (segment D in FIG. 1) can also contain functional and/or regulatory elements that are desired to be incorporated into the final product to act upon or with the cloned nucleic acid insert (segment A in FIG. 1). The subcloning vector can also contain a selectable marker (preferably DNA).

Vector Donor: As used herein, the phrase “Vector Donor” refers to one of the two parental nucleic acid molecules (e.g., RNA or DNA) of the present invention that carries the nucleic acid segments comprising the nucleic acid vector that is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular. A Vector Donor may be referred to as a Destination Vector.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Adapter: As used herein, the term “adapter” refers to an oligonucleotide or nucleic acid fragment or segment (preferably DNA) that comprises one or more recombination sites (or portions of such recombination sites) that can be added to a circular or linear Insert Donor molecule as well as to other nucleic acid molecules described herein. When using portions of recombination sites, the missing portion may be provided by the Insert Donor molecule. Such adapters may be added at any location within a circular or linear molecule, although the adapters are preferably added at or near one or both termini of a linear molecule. Preferably, adapters are positioned to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule that contains the adapter(s) at the site of cleavage. In other aspects, adapters may be added by homologous recombination, by integration of RNA molecules, and the like. Alternatively, adapters may be ligated directly to one or more and preferably both termini of a linear molecule thereby resulting in linear molecule(s) having adapters at one or both termini. In one aspect of the invention, adapters may be added to a population of linear molecules, (e.g., a cDNA library or genomic DNA that has been cleaved or digested) to form a population of linear molecules containing adapters at one and preferably both termini of all or substantial portion of said population.

Adapter-Primer: As used herein, the phrase “adapter-primer” refers to a primer molecule that comprises one or more recombination sites (or portions of such recombination sites) that can be added to a circular or to a linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers are preferably added at or near one or both termini of a linear molecule. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

Template: As used herein, the term “template” refers to a double stranded or single stranded nucleic acid molecule that is to be amplified, synthesized or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, the term “incorporating” means becoming a part of a nucleic acid (e.g., DNA) molecule or primer.

Library: As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), that may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

Amplification: As used herein, the term “amplification” refers to any in vitro method for increasing the number of copies of a nucleic acid molecule with the use of one or more polypeptides having polymerase activity (e.g., one, two, three, four or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Nucleotide: As used herein, the term “nucleotide” refers to a base-sugar -phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids of any length. The terms “peptide,” “oligopeptide,” or “protein” may be used interchangeably herein with the term “polypeptide.”

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

Transduce: As used herein, “transduce” and “transduction” refer to a process of introducing a virus into a cell type that does not support replication of the virus and does not result in the production of infectious viral progeny. In contrast, “infect” or “infection” are used to indicate introduction of a virus into a cell type that supports replication and results in the production of infectious viral progeny.

Other terms used in the fields of recombinant nucleic acid technology and molecular and cell biology as used herein will be generally understood by one of ordinary skill in the applicable arts.

Overview

The present invention relates to methods, compositions and kits for the recombinational joining of two or more segments or nucleic acid molecules to produce a nucleic acid molecule comprising all or a portion of a viral genome, for example, a recombinant viral vector. Further, the present invention relates to methods, compositions and kits for the topoisomerase-mediated joining of two or more segments or nucleic acid molecules to produce a nucleic acid molecule comprising all or a portion of a viral genome, for example, a recombinant viral vector. The present invention also relates to methods, compositions and kits for the joining by other means (e.g., ligase) of two or more segments or nucleic acid molecules to produce a nucleic acid molecule comprising all or a portion of a viral genome, for example, a recombinant viral vector. The invention also includes methods for preparing such nucleic acid molecules, as well as compositions comprising such nucleic acid molecules.

The present invention also contemplates methods for using these molecules to generate host cells, methods of using these molecules to produce polypeptide and/or RNA expression products.

In one embodiment, at least two nucleic acid segments, each comprising at least one recombination site, are contacted with suitable recombination proteins to effect the joining of all or a portion of the two molecules, depending on the position in the molecules of the recombination sites that undergo recombination. Each individual nucleic acid segment may comprise a variety of sequences including, but not limited to viral sequences, sequences suitable for use as primer binding sites (e.g., sequences for which a primer such as a sequencing primer or amplification primer may hybridize to initiate nucleic acid synthesis, amplification or sequencing), transcription or translation signals or regulatory sequences such as promoters and/or enhancers, ribosomal binding sites, Kozak sequences, start codons, termination signals such as stop codons, origins of replication, recombination sites (or portions thereof), selectable markers, and genes or portions of genes to create protein fusions (e.g., N-terminal or C-terminal) such as GST, GUS, GFP, YFP, CFP, maltose binding protein, 6 histidines (HIS6), epitopes, haptens and the like and combinations thereof. The vectors used for cloning such segments may also comprise these functional sequences (e.g., promoters, primer sites, etc.).

After joining of the segments, the product molecule will often contain at least sufficient viral sequences to permit the packaging of the product molecule in a viral particle. In the case where the viral sequences are adenoviral sequences, the product molecule may contain a left ITR, a packaging sequence and a right ITR, and/or sufficient other sequences to result in a molecule of appropriate size for packaging. In some embodiments, the product molecule comprises sufficient viral sequences to be an infectious viral genome when introduced into a permissive host cell. In some embodiments, a recombinant adenoviral vector produced by the methods of the invention may comprise a left ITR, a packaging sequence a first recombination site, a sequence of interest, a second recombination site, and additional adenoviral sequences including a right ITR. In the case where the viral sequences are retroviral sequences, the product molecule may contain a 5′-LTR, a 3′-LTR and a packaging sequence (Ψ), and/or sufficient other sequences to result in a molecule of appropriate size for packaging. In some embodiments, the product molecule comprises sufficient retroviral sequences to integrate into the genome of host cell into which it is introduced but not enough viral sequences to produce an infectious virus in the host cell. In some embodiments, a recombinant retroviral vector produced by the methods of the invention may be a plasmid comprising a 5′-LTR, a packaging sequence a first recombination site, a sequence of interest, and a second recombination site, and additional retroviral sequences including a 3′-LTR.

Recombination Sites

Recombination sites for use in the invention may be any nucleic acid that can serve as a substrate in a recombination reaction. Such recombination sites may be wild-type or naturally occurring recombination sites, or modified, variant, derivative, or mutant recombination sites. Examples of recombination sites for use in the invention include, but are not limited to, phage-lambda recombination sites (such as attP, attB, attL, and attR and mutants or derivatives thereof) and recombination sites from other bacteriophages such as phi80, P22, P2, 186, P4 and P1 (including lox sites such as loxP and loxP511).

In some embodiments, recombination sites that may be used in the practice of the invention include recombination sites that undergo recombination with compatible recombination sites in the presence of one or more recombination proteins active in the phage lambda recombination system, for example, one or more of Int, IHF, FIS, and/or Xis. The invention also contemplates nucleic acid molecules comprising such recombination sites and compositions comprising such nucleic acid molecules. Preferred recombination proteins and mutant, modified, variant, or derivative recombination sites for use in the invention include those described in U.S. Pat. Nos. 5,888,732, 6,143,557, 6,171,861, 6,270,969, and 6,277,608 and in U.S. application Ser. No. 09/438,358 (filed Nov. 12, 1999), based upon U.S. provisional application No. 60/108,324 (filed Nov. 13, 1998). Mutated att sites (e.g., attB 1-10, attP 1-10, attR 1-10 and attL 1-10) are described in U.S. application Ser. No. 09/517,466, filed Mar. 2, 2000, and Ser. No. 09/732,914, filed Dec. 11, 2000 (published as 2002 0007051-A1) the disclosures of which are specifically incorporated herein by reference in their entirety. Other suitable recombination sites and proteins are those associated with the GATEWAY™ Cloning Technology available from Invitrogen Corporation, Carlsbad, Calif., and described in the product literature of the GATEWAY™ Cloning Technology, the entire disclosures of all of which are specifically incorporated herein by reference in their entireties.

Sites that may be used in the present invention include att sites. The 15 bp core region of the wildtype att site (GCTTTTTTAT ACTAA (SEQ ID NO:)), which is identical in all wildtype att sites, may be mutated in one or more positions. Other att sites that specifically recombine with other att sites can be constructed by altering nucleotides in and near the 7 base pair overlap region, bases 6-12 of the core region. Thus, recombination sites suitable for use in the methods, molecules, compositions, and vectors of the invention include, but are not limited to, those with insertions, deletions or substitutions of one, two, three, four, or more nucleotide bases within the 15 base pair core region (see U.S. application Ser. No. 08/663,002, filed Jun. 7, 1996 (now U.S. Pat. No. 5,888,732) and Ser. No. 09/177,387, filed Oct. 23, 1998, which describes the core region in further detail, and the disclosures of which are incorporated herein by reference in their entireties). Recombination sites suitable for use in the methods, compositions, and vectors of the invention also include those with insertions, deletions or substitutions of one, two, three, four, or more nucleotide bases within the 15 base pair core region that are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

As a practical matter, whether any particular nucleic acid molecule is at least 50%, 60%, 70%, 75%, 80%, 85%, 90%, 95%, 96%, 97%, 98% or 99% identical to, for instance, a given recombination site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

Analogously, the core regions in attB1, attP1, attL1 and attR1 are identical to one another, as are the core regions in attB2, attP2, attL2 and attR2. Nucleic acid molecules suitable for use with the invention also include those comprising insertions, deletions or substitutions of one, two, three, four, or more nucleotides within the seven base pair overlap region (TTTATAC, bases 6-12 in the core region). The overlap region is defined by the cut sites for the integrase protein and is the region where strand exchange takes place. Examples of such mutants, fragments, variants and derivatives include, but are not limited to, nucleic acid molecules in which (1) the thymine at position 1 of the seven bp overlap region has been deleted or substituted with a guanine, cytosine, or adenine; (2) the thymine at position 2 of the seven bp overlap region has been deleted or substituted with a guanine, cytosine, or adenine; (3) the thymine at position 3 of the seven bp overlap region has been deleted or substituted with a guanine, cytosine, or adenine; (4) the adenine at position 4 of the seven bp overlap region has been deleted or substituted with a guanine, cytosine, or thymine; (5) the thymine at position 5 of the seven bp overlap region has been deleted or substituted with a guanine, cytosine, or adenine; (6) the adenine at position 6 of the seven bp overlap region has been deleted or substituted with a guanine, cytosine, or thymine; and (7) the cytosine at position 7 of the seven bp overlap region has been deleted or substituted with a guanine, thymine, or adenine; or any combination of one or more (e.g., two, three, four, five, etc.) such deletions and/or substitutions within this seven bp overlap region. The nucleotide sequences of representative seven base pair core regions are set out below.

Altered att sites have been constructed that demonstrate that (1) substitutions made within the first three positions of the seven base pair overlap (TTTATAC) strongly affect the specificity of recombination, (2) substitutions made in the last four positions (TTTATAC) only partially alter recombination specificity, and (3) nucleotide substitutions outside of the seven bp overlap, but elsewhere within the 15 base pair core region, do not affect specificity of recombination but do influence the efficiency of recombination. Thus, nucleic acid molecules and methods of the invention include those comprising or employing one, two, three, four, five, six, eight, ten, or more recombination sites which affect recombination specificity, particularly one or more (e.g., one, two, three, four, five, six, eight, ten, twenty, thirty, forty, fifty, etc.) different recombination sites that may correspond substantially to the seven base pair overlap within the 15 base pair core region, having one or more mutations that affect recombination specificity. Particularly preferred such molecules may comprise a consensus sequence such as NNNATAC wherein “N” refers to any nucleotide (i.e., may be A, G, T/U or C). Preferably, if one of the first three nucleotides in the consensus sequence is a T/U, then at least one of the other two of the first three nucleotides is not a T/U.

The core sequence of each att site (attB, attP, attL and attR) can be divided into functional units consisting of integrase binding sites, integrase cleavage sites and sequences that determine specificity. Specificity determinants are defined by the first three positions following the integrase top strand cleavage site. These three positions are shown with underlining in the following reference sequence: CAACTTTTTTATAC AAAGTTG (SEQ ID NO:______). Modification of these three positions (64 possible combinations) can be used to generate att sites that recombine with high specificity with other att sites having the same sequence for the first three nucleotides of the seven base pair overlap region. The possible combinations of first three nucleotides of the overlap region are shown in Table 1.

TABLE 1

Modifications of the First Three Nucleotides of
the att Site Seven Base Pair Overlap Region that
Alter Recombination Specificity.

| | | | |
|---|---|---|---|
| AAA | CAA | GAA | TAA |
| AAC | CAC | GAC | TAC |
| AAG | CAG | GAG | TAG |
| AAT | CAT | GAT | TAT |
| ACA | CCA | GCA | TCA |
| ACC | CCC | GCC | TCC |
| ACG | CCG | GCG | TCG |
| ACT | CCT | GCT | TCT |
| AGA | CGA | GGA | TGA |
| AGC | CGC | GGC | TGC |
| AGG | CGG | GGG | TGG |
| AGT | CGT | GGT | TGT |
| ATA | CTA | GTA | TTA |
| ATC | CTC | GTC | TTC |
| ATG | CTG | GTG | TTG |
| ATT | CTT | GTT | TTT |

Representative examples of seven base pair att site overlap regions suitable for in methods, compositions and vectors of the invention are shown in Table 2. The invention further includes nucleic acid molecules comprising one or more (e.g., one, two, three, four, five, six, eight, ten, twenty, thirty, forty, fifty, etc.) nucleotides sequences set out in Table 2. Thus, for example, in one aspect, the invention provides nucleic acid molecules comprising the nucleotide sequence GAAATAC, GATATAC, ACAATAC, or TGCATAC.

TABLE 2

Representative Examples of Seven Base Pair att
Site Overlap Regions Suitable for use in the
recombination sites of the Invention.

| | | | |
|---|---|---|---|
| AAAATAC | CAAATAC | GAAATAC | TAAATAC |
| AACATAC | CACATAC | GACATAC | TACATAC |
| AAGATAC | CAGATAC | GAGATAC | TAGATAC |
| AATATAC | CATATAC | GATATAC | TATATAC |
| ACAATAC | CCAATAC | GCAATAC | TCAATAC |
| ACCATAC | CCCATAC | GCCATAC | TCCATAC |
| ACGATAC | CCGATAC | GCGATAC | TCGATAC |
| ACTATAC | CCTATAC | GCTATAC | TCTATAC |
| AGAATAC | CGAATAC | GGAATAC | TGAATAC |
| AGCATAC | CGCATAC | GGCATAC | TGCATAC |
| AGGATAC | CGGATAC | GGGATAC | TGGATAC |
| AGTATAC | CGTATAC | GGTATAC | TGTATAC |
| ATAATAC | CTAATAC | GTAATAC | TTAATAC |
| ATCATAC | CTCATAC | GTCATAC | TTCATAC |
| ATGATAC | CTGATAC | GTGATAC | TTGATAC |
| ATTATAC | CTTATAC | GTTATAC | TTTATAC |

As noted above, alterations of nucleotides located 3′ to the three base pair region discussed above can also affect recombination specificity. For example, alterations within the last four positions of the seven base pair overlap can also affect recombination specificity.

For example, mutated att sites that may be used in the practice of the present invention include attB1 (AGCCTGCTTT TTTGTACAAA CTTGT (SEQ ID NO: )), attP1 (TACAGGTCAC TAATACCATC TAAGTAGTTG ATTCATAGTG ACTGGATATG TTGTGTTTTA CAGTATTATG TAGTCTGTTT TTTATGCAAA ATCTAATTTA ATATATTGAT ATTTATATCA TTTTACGTTT CTCGTTCAGC TTTTTTGTAC AAAGTTGGCA TTATAAAAAA GCATTGCTCA TCAATTTGTT GCAACGAACA GGTCACTATC AGTCAAAATA AAATCATTAT TTG (SEQ ID NO: )), attL1 (CAAATAATGA TTTTATTTTG ACTGATAGTG ACCTGTTCGT TGCAACAAAT TGATAAGCAA TGCTTTTTTA TAATGCCAAC TTTGTACAAA AAAGCAGGCT (SEQ ID NO:)), and attR1 (ACAAGTTTGT ACAAAAAAGC TGAACGAGAA ACGTAAAATG ATATAAATAT CAATATATTA AATTAGATTT TGCATAAAAA ACAGACTACA TAATACTGTA AAACACAACA TATCCAGTCA CTATG (SEQ ID NO: )). Table 3 provides the sequences of the regions surrounding the core region for the wild type att sites (attB0, P0, R0, and L0) as well as a variety of other suitable recombination sites. Those skilled in the art will appreciated that the remainder of the site may be the same as the corresponding site (B, P, L, or R) listed above.

TABLE 3

| Nucleotide sequences of att sites. | | |
|---|---|---|
| attB0 | AGCCTGCTTT TTTATACTAA CTTGAGC | (SEQ ID NO: ) |
| attP0 | GTTCAGCTTT TTTATACTAA GTTGGCA | (SEQ ID NO: ) |
| attL0 | AGCCTGCTTT TTTATACTAA GTTGGCA | (SEQ ID NO: ) |
| attR0 | GTTCAGCTTT TTTATACTAA CTTGAGC | (SEQ ID NO: ) |
| attB1 | AGCCTGCTTT TTTGTACAAA CTTGT | (SEQ ID NO: ) |
| attP1 | GTTCAGCTTT TTTGTACAAA GTTGGCA | (SEQ ID NO: ) |
| attL1 | AGCCTGCTTT TTTGTACAAA GTTGGCA | (SEQ ID NO: ) |
| attR1 | GTTCAGCTTT TTTGTACAAA CTTGT | (SEQ ID NO: ) |
| attB2 | ACCCAGCTTT CTTGTACAAA GTGGT | (SEQ ID NO: ) |
| attP2 | GTTCAGCTTT CTTGTACAAA GTTGGCA | (SEQ ID NO: ) |
| attL2 | ACCCAGCTTT CTTGTACAAA GTTGGCA | (SEQ ID NO: ) |
| attR2 | GTTCAGCTTT CTTGTACAAA GTGGT | (SEQ ID NO: ) |
| attB5 | CAACTTTATT ATACAAAGTT GT | (SEQ ID NO: ) |
| attP5 | GTTCAACTTT ATTATACAAA GTTGGCA | (SEQ ID NO: ) |
| attL5 | CAACTTTATT ATACAAAGTT GGCA | (SEQ ID NO: ) |
| attR5 | GTTCAACTTT ATTATACAAA GTTGT | (SEQ ID NO: ) |
| attB11 | CAACTTTTCT ATACAAAGTT GT | (SEQ ID NO: ) |
| attP11 | GTTCAACTTT TCTATACAAA GTTGGCA | (SEQ ID NO: ) |
| attL11 | CAACTTTTCT ATACAAAGTT GGCA | (SEQ ID NO: ) |
| attR11 | GTTCAACTTT TCTATACAAA GTTGT | (SEQ ID NO: ) |
| attB17 | CAACTTTTGT ATACAAAGTT GT | (SEQ ID NO: ) |
| attP17 | GTTCAACTTT TGTATACAAA GTTGGCA | (SEQ ID NO: ) |
| attL17 | CAACTTTTGT ATACAAAGTT GGCA | (SEQ ID NO: ) |
| attR17 | GTTCAACTTT TGTATACAAA GTTGT | (SEQ ID NO: ) |
| attB19 | CAACTTTTTC GTACAAAGTT GT | (SEQ ID NO: ) |
| attP19 | GTTCAACTTT TTCGTACAAA GTTGGCA | (SEQ ID NO: ) |
| attL19 | CAACTTTTTC GTACAAAGTT GGCA | (SEQ ID NO: ) |
| attR19 | GTTCAACTTT TTCGTACAAA GTTGT | (SEQ ID NO: ) |
| attB20 | CAACTTTTTG GTACAAAGTT GT | (SEQ ID NO: ) |
| attP20 | GTTCAACTTT TTGGTACAAA GTTGGCA | (SEQ ID NO: ) |
| attL20 | CAACTTTTTG GTACAAAGTT GGCA | (SEQ ID NO: ) |
| attR20 | GTTCAACTTT TTGGTACAAA GTTGT | (SEQ ID NO: ) |
| attB21 | CAACTTTTTA ATACAAAGTT GT | (SEQ ID NO: ) |
| attP21 | GTTCAACTTT TTAATACAAA GTTGGCA | (SEQ ID NO: ) |
| attL21 | CAACTTTTTA ATACAAAGTT GGCA | (SEQ ID NO: ) |
| attR21 | GTTCAACTTT TTAATACAAA GTTGT | (SEQ ID NO: ) |

Other recombination sites having unique specificity (i.e., a first site will recombine with its corresponding site and will not substantially recombine with a second site having a different specificity) are known to those skilled in the art and may be used to practice the present invention.

Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., γδ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. No. 5,851,808 issued to Elledge and Liu which is specifically incorporated herein by reference.

The materials and methods of the invention may further encompass the use of “single use” recombination sites which undergo recombination one time and then either undergo recombination with low frequency (e.g., have at least five fold, at least ten fold, at least fifty fold, at least one hundred fold, or at least one thousand fold lower recombination activity in subsequent recombination reactions) or are essentially incapable of undergoing recombination. The invention also provides methods for making and using nucleic acid molecules which contain such single use recombination sites and molecules which contain these sites. Examples of methods which can be used to generate and identify such single use recombination sites are set out below. Further examples of methods which can be used to generate and identify such single use recombination sites are set out in PCT/US00/21623, published as WO 01/11058, which claims priority to U.S. provisional patent application 60/147,892, filed Aug. 9, 1999, both of which are specifically incorporated herein by reference.

The att system core integrase binding site comprises an interrupted seven base pair inverted repeat having the following nucleotide sequence:

------> . . . <------

caactttnnnnnnnaaagttg (SEQ ID NO:39),

as well as variations thereof which can comprise either perfect or imperfect repeats.

The repeat elements can be subdivided into two distal and/or proximal “domains” composed of caac/gttg segments (underlined), which are distal to the central undefined sequence (the nucleotides of which are represented by the letter “n”), and ttt/aaa segments, which are proximal to the central undefined sequence.

Alterations in the sequence composition of the distal and/or proximal domains on one or both sides of the central undefined region can affect the outcome of a recombination reaction. The scope and scale of the effect is a function of the specific alterations made, as well as the particular recombinational event (e.g., LR vs. BP reactions).

For example, it is believed that an attB site altered to have the following nucleotide sequence:

------> . . . <------

caactttnnnnnnnaaacaag (SEQ ID NO:40),

will functionally interact with a cognate attP and generate attL and attR. However, whichever of the latter two recombination sites acquires the segment containing “caag” (located on the left side of the sequence shown above) will be rendered non-functional to subsequent recombination events. The above is only one of many possible alterations in the core integrase binding sequence which can render att sites non-functional after engaging in a single recombination event. Thus, single use recombination sites may be prepared by altering nucleotides in the seven base pair inverted repeat regions which abut seven base pair overlap regions of att sites. This region is represented schematically as:

CAAC TTT [Seven Base Pair Overlap Region] AAA GTTG.

In generating single use recombination sites, one, two, three, four or more of nucleotides of the sequences CAACTTT or AAAGTTG (i.e., the seven base pair inverted repeat regions) may be substituted with other nucleotides or deleted altogether. These seven base pair inverted repeat regions represent complementary sequences with respect to each other. Thus, alterations may be made in either seven base pair inverted repeat region in order to generate single use recombination sites. Further, when DNA is double stranded and one seven base pair inverted repeat region is present, the other seven base pair inverted repeat region will also be present on the other strand.

Using the sequence CAACTTT for illustration, examples of seven base pair inverted repeat regions which can form single use recombination sites include, but are not limited to, nucleic acid molecules in which (1) the cytosine at position 1 of the seven base pair inverted repeat region has been deleted or substituted with a guanine, adenine, or thymine; (2) the adenine at position 2 of the seven base pair inverted repeat region has been deleted or substituted with a guanine, cytosine, or thymine; (3) the adenine at position 3 of the seven base pair inverted repeat region has been deleted or substituted with a guanine, cytosine, or thymine; (4) the cytosine at position 4 of the seven base pair inverted repeat region has been deleted or substituted with a guanine, adenine, or thymine; (5) the thymine at position 5 of the seven base pair inverted repeat region has been deleted or substituted with a guanine, cytosine, or adenine; (6) the thymine at position 6 of the seven base pair inverted repeat region has been deleted or substituted with a guanine, cytosine, or adenine; and (7) the thymine at position 7 of the seven base pair inverted repeat region has been deleted or substituted with a guanine, cytosine, or adenine; or any combination of one, two, three, four, or more such deletions and/or substitutions within this seven base pair region. Representative examples of nucleotide sequences of the above described seven base pair inverted repeat regions are set out below in Table 4.

TABLE 4

Representative examples of nucleotide sequences
of seven base pair inverted repeat regions.

| | | | |
|---|---|---|---|
| aagaaaa | aagagcg | aagagaa | aagatat |
| ccgccac | ccgcctc | ccgcaca | ccgcttt |
| ggtggga | ggtgctc | ggtgata | ggtgtat |
| ttctttg | ttctctc | ttctgaa | ttctttt |
| aatacac | aatagcg | aataaca | aatatat |
| cctcgga | cctcccg | cctcaca | cctcttt |
| ggcgaaa | ggcgccg | ggcggaa | ggcgtat |
| ttgtcac | ttgtgcg | ttgtaca | ttgtttt |
| acaagga | acaaccg | acaaata | acaattt |
| caccttg | caccaga | caccgaa | cacctat |
| gaggcac | gagggcg | gaggaca | gaggttt |
| tattgga | tattaga | tattaca | tatttat |
| agaaaaa | agaaaga | agaagaa | agaattt |
| cgcccac | cgccctc | cgccaca | cgccttt |
| gcgggga | gcgggcg | gcggata | gcggtat |
| tcttttg | tcttccg | tcttgaa | tcttttt |
| ataacac | ataactc | ataaaca | ataattt |
| ctccaaa | ctccgcg | ctccata | ctcctat |
| gtgggga | gtggccg | gtgggaa | gtggtat |
| tgttttg | tgttctc | tgttaca | tgttttt |

Representative examples of nucleotide sequences which form single use recombination sites may also be prepared by combining a nucleotide sequence set out in Table 5, Section 1, with a nucleotide sequence set out in Table 5, Section 2. Single use recombination sites may also be prepared by the insertion of one or more (e.g., one, two, three, four, five six, seven, etc.) nucleotides internally within these regions.

TABLE 5

Representative examples of nucleotide
sequences which form single use
recombination sites.

| | |
|---|---|
| Section 1 (CAAG) | Section 2 (TTT) |

| | |
|---|---|
| aaaa cccc gggg tttt | aaa cca ttc |
| aaac ccca ggga ttta | aac cac ttg |
| aaag ccct gggc tttc | aag cgc tat |
| aaat cccg gggt tttg | aat ctc tct |
| aaca ccac ggag ttat | aca ggg tgt |
| aaga ccgc ggtg ttct | aga gga |
| aata cctc ggcg ttgt | ata ggc |
| acaa cacc gagg tatt | caa ggt |
| agaa cgcc gcgg tctt | gaa gag |
| ataa ctcc gtgg tgtt | taa gcg |
| caaa accc aggg attt | ccc gtg |
| gaaa gccc CGG cttt | ccg ttt |
| taaa tccc tggg gttt | cct tta |

In most instances where one seeks to prevent recombination events with respect to a particular nucleic acid segment, the altered sequence will be located proximally to the nucleic acid segment. Using the following schematic for illustration:

=5′ Nucleic Acid Segment 3′=caac ttt (Seven Base Pair Overlap Region) AAA GTTG,

the lower case nucleotide sequence which represent a seven base pair inverted repeat region (i.e., caac ttt) will generally have a sequence altered by insertion, deletion, and/or substitution to form a single use recombination site when one seeks to prevent recombination at the 3′ end (i.e., proximal end with respect to the nucleic acid segment) of the nucleic acid segment shown. Thus, a single recombination reaction can be used, for example, to integrate the nucleic acid segments into another nucleic acid molecule, then the recombination site becomes effectively non-functional, preventing the site from engaging in further recombination reactions. Similarly, single use recombination sites can be position at both ends of a nucleic acid segment so that the nucleic acid segment can be integrated into another nucleic acid molecule, or circularized, and will remain integrated, or circularized even in the presence of recombinases.

A number of methods may be used to screen potential single use recombination sites for functional activity (e.g., undergo one recombination event followed by the failure to undergo subsequent recombination events). For example, with respect to the screening of recombination sites to identify those which become non-functional after a single recombination event, a first recombination reaction may be performed to generate a plasmid in which a negative selection marker is linked to one or more potentially defective recombination sites. The plasmid may then be reacted with another nucleic acid molecule which comprises a positive selection marker similarly linked to recombination sites. Thus, this selection system is designed such that molecules which recombine are susceptible to negative selection and molecules which do not recombine may be selected for by positive selection. Using such a system, one may then directly select for desired single use core site mutants.

As one skilled in the art would recognize, any number of screening assays may be designed which achieve the same results as those described above. In many instances, these assays will be designed so that an initial recombination event takes place and then recombination sites which are unable to engage in subsequent recombination events are identified or molecules which contain such recombination sites are selected for. A related screening assay would result in selection against nucleic acid molecule which have undergone a second recombination event. Further, as noted above, screening assays can be designed where there is selection against molecules which have engaged in subsequent recombination events and selection for those which have not engaged in subsequent recombination events.

Single use recombination sites are especially useful for either decreasing the frequency of or preventing recombination when either large number of nucleic acid segments are attached to each other or multiple recombination reactions are performed. Thus, the invention further includes nucleic acid molecules which contain single use recombination sites, as well as methods for performing recombination using these sites.

Recombination sites used with the invention may also have embedded functions or properties. An embedded functionality is a function or property conferred by a nucleotide sequence in a recombination site that is not directly associated with recombination efficiency or specificity. For example, recombination sites may contain protein coding sequences (e.g., intein coding sequences), intron/exon splice sites, origins of replication, and/or stop codons. Further, recombination sites that have more than one (e.g., two, three, four, five, etc.) embedded functions or properties may also be prepared.

In some instances it will be advantageous to remove either RNA corresponding to recombination sites from RNA transcripts or amino acid residues encoded by recombination sites from polypeptides translated from such RNAs. Removal of such sequences can be performed in several ways and can occur at either the RNA or protein level. One instance where it may be advantageous to remove RNA transcribed from a recombination site will be when constructing a fusion polypeptide between a polypeptide of interest and a coding sequence present on the vector. The presence of an intervening recombination site between the ORF of the polypeptide of interest and the vector coding sequences may result in the recombination site (1) contributing codons to the mRNA that result in the inclusion of additional amino acid residues in the expression product, (2) contributing a stop codon to the mRNA that prevents the production of the desired fusion protein, and/or (3) shifting the reading frame of the mRNA such that the two protein are not fused “in-frame.”

In one aspect, the invention provides methods for removing nucleotide sequences encoded by recombination sites from RNA molecules. One example of such a method employs the use of intron/exon splice sites to remove RNA encoded by recombination sites from RNA transcripts. Nucleotide sequences that encode intron/exon splice sites may be fully or partially embedded in the recombination sites used in the present invention and/or may encoded by adjacent nucleic acid sequence. Sequences to be excised from RNA molecules may be flanked by splice sites that are appropriately located in the sequence of interest and/or on the vector. For example, one intron/exon splice site may be encoded by a recombination site and another intron/exon splice site may be encoded by other nucleotide sequences (e.g., nucleic acid sequences of the vector or a nucleic acid of interest). Nucleic acid splicing is well known to those skilled in the art and is discussed in the following publications: R. Reed, Curr. Opin. Genet. Devel. 6:215-220 (1996); S. Mount, Nucl. Acids. Res. 10:459-472, (1982); P. Sharp, Cell 77:805-815, (1994); K. Nelson and M. Green, Genes and Devel. 23:319-329 (1988); and T. Cooper and W. Mattox, Am. J. Hum. Genet. 61:259-266 (1997).

Splice sites can be suitably positioned in a number of locations. For example, a Destination Vector designed to express an inserted ORF with an N-terminal fusion—for example, with a detectable marker—the first splice site could be encoded by vector sequences located 3′ to the detectable marker coding sequences and the second splice site could be partially embedded in the recombination site that separates the detectable marker coding sequences from the coding sequences of the ORF. Further, the second splice site either could abut the 3′ end of the recombination site or could be positioned a short distance (e.g., 2, 4, 8, 10, 20 nucleotides) 3′ to the recombination site. In addition, depending on the length of the recombination site, the second splice site could be fully embedded in the recombination site.

A modification of the method described above involves the connection of multiple nucleic acid segments that, upon expression, results in the production of a fusion protein. In one specific example, one nucleic acid segment encodes detectable marker—for example, GFP—and another nucleic acid segment that encodes an ORF of interest. Each of these segments is flanked by recombination sites. In addition, the nucleic acid segments that encodes the detectable marker contains an intron/exon splice site near its 3′ terminus and the nucleic acid segments that contains the ORF of interest also contains an intron/exon splice site near its 5′ terminus. Upon recombination, the nucleic acid segment that encodes the detectable marker is positioned 5′ to the nucleic acid segment that encodes the ORF of interest. Further, these two nucleic acid segments are separated by a recombination site that is flanked by intron/exon splice sites. Excision of the intervening recombination site thus occurs after transcription of the fuision mRNA. Thus, in one aspect, the invention is directed to methods for removing RNA transcribed from recombination sites from transcripts generated from nucleic acids described herein.

Splice sites may introduced into nucleic acid molecules to be used in the present invention in a variety of ways. One method that could be used to introduce intron/exon splice sites into nucleic acid segments is PCR. For example, primers could be used to generate nucleic acid segments corresponding to an ORF of interest and containing both a recombination site and an intron/exon splice site.

The above methods can also be used to remove RNA corresponding to recombination sites when the nucleic acid segment that is recombined with another nucleic acid segment encodes RNA that is not produced in a translatable format. One example of such an instance is where a nucleic acid segment is inserted into a vector in a manner that results in the production of antisense RNA. As discussed below, this antisense RNA may be fused, for example, with RNA that encodes a ribozyme. Thus, the invention also provides methods for removing RNA corresponding to recombination sites from such molecules.

The invention further provides methods for removing amino acid sequences encoded by recombination sites from protein expression products by protein splicing. Nucleotide sequences that encode protein splice sites may be fully or partially embedded in the recombination sites that encode amino acid sequences excised from proteins or protein splice sites may be encoded by adjacent nucleotide sequences. Similarly, one protein splice site may be encoded by a recombination site and another protein splice sites may be encoded by other nucleotide sequences (e.g., nucleic acid sequences of the vector or a nucleic acid of interest).

It has been shown that protein splicing can occur by excision of an intein from a protein molecule and ligation of flanking segments (see, e.g., Derbyshire, et al., Proc. Natl. Acad. Sci. (USA) 95:1356-1357 (1998)). In brief, inteins are amino acid segments that are post-translationally excised from proteins by a self-catalytic splicing process. A considerable number of intein consensus sequences have been identified (see, e.g., Perler, Nucleic Acids Res. 27:346-347 (1999)).

Similar to intron/exon splicing, N- and C-terminal intein motifs have been shown to be involved in protein splicing. Thus, the invention further provides compositions and methods for removing amino acid residues encoded by recombination sites from protein expression products by protein splicing. In particular, this aspect of the invention is related to the positioning of nucleic acid sequences that encode intein splice sites on both the 5′ and 3′ end of recombination sites positioned between two coding regions. Thus, when the protein expression product is incubated under suitable conditions, amino acid residues encoded these recombination sites will be excised.

Protein splicing may be used to remove all or part of the amino acid sequences encoded by recombination sites. Nucleic acid sequence that encode inteins may be fully or partially embedded in recombination sites or may adjacent to such sites. In certain circumstances, it may be desirable to remove considerable numbers of amino acid residues beyond the N- and/or C-terminal ends of amino acid sequences encoded by recombination sites. In such instances, intein coding sequence may be located a distance (e.g., 30, 50, 75, 100, etc. nucleotides) 5′ and/or 3′ to the recombination site.

While conditions suitable for intein excision will vary with the particular intein, as well as the protein that contains this intein, Chong, et al., Gene 192:271-281 (1997), have demonstrated that a modified Saccharomyces cerevisiae intein, referred to as Sce VMA intein, can be induced to undergo self-cleavage by a number of agents including 1,4-dithiothreitol (DTT), β-mercaptoethanol, and cysteine. For example, intein excision/splicing can be induced by incubation in the presence of 30 mM DTT, at 4° C. for 16 hours.

Topoisomerase Cloning

The present invention also relates to methods of using one or more topoisomerases to generate a recombinant nucleic acid molecules of the invention (e.g., molecules comprising all or a portion of a viral genome such as a viral vector) comprising two or more nucleotide sequences, any one or more of which may comprise, for example, all or a portion of a viral genome. Topoisomerases may be used in combination with recombinational cloning techniques described above. For example, a topoisomerase-mediated reaction may be used to attach one or more recombination sites to one or more nucleic acid segments. The segments may then be further manipulated and combined using, for example, recombinational cloning techniques.

In one aspect, the present invention provides methods for linking a first and at least a second nucleic acid segment (either or both of which may contain viral sequences and/or sequences of interest) with at least one (e.g., 1, 2, 3, 4, 5, 6, 7, 8, 9, 10, etc.) topoisomerase (e.g., a type IA, type IB, and/or type II topoisomerase) such that either one or both strands of the linked segments are covalently joined at the site where the segments are linked.

A method for generating a double stranded recombinant nucleic acid molecule covalently linked in one strand can be performed by contacting a first nucleic acid molecule which has a site-specific topoisomerase recognition site (e.g., a type IA or a type II topoisomerase recognition site), or a cleavage product thereof, at a 5′ or 3′ terminus, with a second (or other) nucleic acid molecule, and optionally, a topoisomerase (e.g., a type IA, type IB, and/or type II topoisomerase), such that the second nucleotide sequence can be covalently attached to the first nucleotide sequence. As disclosed herein, the methods of the invention can be performed using any number of nucleotide sequences, typically nucleic acid molecules wherein at least one of the nucleotide sequences has a site-specific topoisomerase recognition site (e.g., a type IA, type IB or type II topoisomerase), or cleavage product thereof, at one or both 5′ and/or 3′ termini.

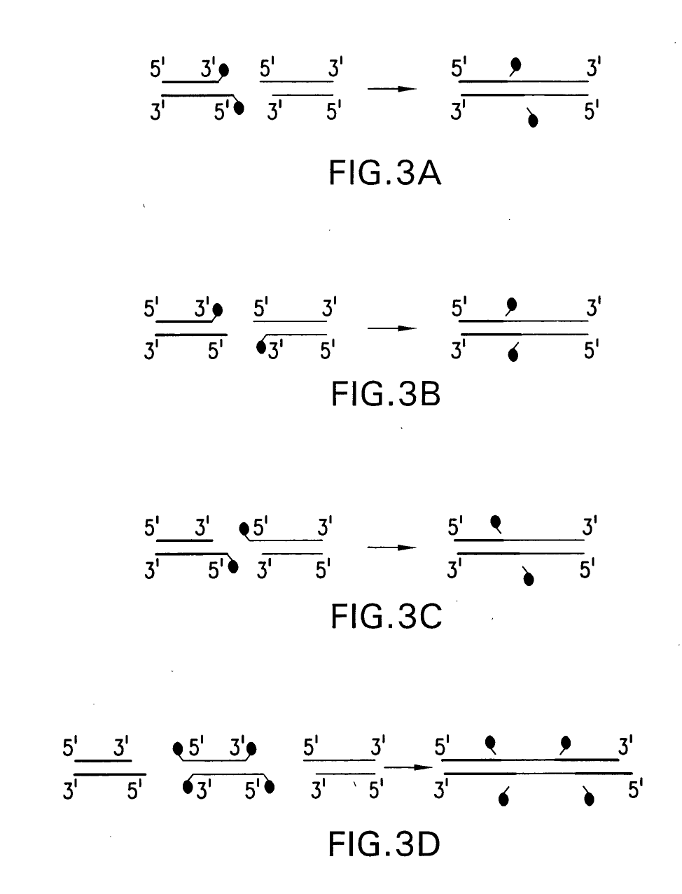

In some embodiments, two double-stranded nucleic acid molecules can be joined into a one larger molecule such that each strand of the larger molecule is covalently joined (e.g., the larger molecule has no nicks). With reference to FIG. 3, a first double-stranded nucleic acid molecule having a topoisomerase linked to each of the 5′ terminus and 3′ terminus of one end may be contacted with a second nucleic acid under conditions causing the linkage of both strands of the first nucleic acid molecule to both strands of the second nucleic acid molecule (FIG. 3A). The end of the first nucleic acid molecules to which the topoisomerases are attached may have either a 5′-overhang, 3′-overhang or be blunt ended. The end of the second nucleic acid molecule to be joined to the first nucleic acid molecule may have the same type of end as the topoisomerase-linked end of the first nucleic acid molecule. The end of the second molecule that is not to be joined may have a different end if directional joining of the segments is desired and may have the same type of end if directionality is not required.

In another embodiment, a first nucleic acid molecule having a topoisomerase bound to the 3′ terminus of one end, and a second nucleic acid molecule having a topoisomerase bound to the 3′ terminus of one end may be joined using the methods of the invention (FIG. 3B). A covalently linked double-stranded recombinant nucleic acid molecule is generated by contacting the ends containing the topoisomerase-charged substrate nucleic acid molecules.

FIG. 3C shows a first nucleic acid molecule having a topoisomerase bound to the 5′ terminus of one end, and a second nucleic acid molecule having a topoisomerase bound to the 5′ terminus of one end, and further shows the production of a covalently linked double-stranded recombinant nucleic acid molecule generated by contacting the ends containing the topoisomerase-charged substrate nucleic acid molecules.

FIG. 3D shows a nucleic acid molecule having a topoisomerase linked to each of the 5′ terminus and 3′ terminus of both ends, and further shows linkage of the topoisomerase-charged nucleic acid molecule to two nucleic acid molecules, one at each end. The topoisomerases at each of the 5′ termini and/or at each of the 3′ termini can be the same or different. Those skilled in the art will appreciate that nicked molecules (e.g., covalently joined in only one strand) may be produced by omitting one of the topoisomerases from the any one of the methods described above for FIGS. 3A-3D.

A method for generating a double stranded recombinant nucleic acid molecule covalently linked in both strands can be performed, for example, by contacting a first nucleic acid molecule having a first end and a second end, wherein, at the first end or second end or both ends, the first nucleic acid molecule has a topoisomerase recognition site (or cleavage product thereof) at or near the 5′ or 3′ terminus; at least a second nucleic acid molecule having a first end and a second end, wherein, at the first end or second end or both ends, the at least second double stranded nucleotide sequence has a topoisomerase recognition site (or cleavage product thereof) at or near a 5′ or 3′ terminus; and at least one site specific topoisomerase (e.g., a type IA and/or a type IB topoisomerase), under conditions such that all components are in contact and the topoisomerase can effect its activity. A covalently linked double stranded recombinant nucleic acid generated according to a method of this aspect of the invention is characterized, in part, in that it does not contain a nick in either strand at the position where the nucleic acid molecules are joined. In one embodiment, the method is performed by contacting a first nucleic acid molecule and a second (or other) nucleic acid molecule, each of which has a topoisomerase recognition site in addition to viral sequences an/or sequences of interest, or a cleavage product thereof, at the 3′ termini or at the 5′ termini of two ends to be covalently linked. In another embodiment, the method is performed by contacting a first nucleic acid molecule having a topoisomerase recognition site, or cleavage product thereof, at the 5′ terminus and the 3′ terminus of at least one end, and a second (or other) nucleic acid molecule having a 3′ hydroxyl group and a 5′ hydroxyl group at the end to be linked to the end of the first nucleic acid molecule containing the recognition sites. As disclosed herein, the methods can be performed using any number of nucleic acid molecules having various combinations of termini and ends.

Topoisomerases are categorized as type I, including type IA and type IB topoisomerases, which cleave a single strand of a double stranded nucleic acid molecule, and type II topoisomerases (gyrases), which cleave both strands of a nucleic acid molecule. Type IA and IB topoisomerases cleave one strand of a nucleic acid molecule. Cleavage of a nucleic acid molecule by type IA topoisomerases generates a 5′ phosphate and a 3′ hydroxyl at the cleavage site, with the type IA topoisomerase covalently binding to the 5′ terminus of a cleaved strand. In comparison, cleavage of a nucleic acid molecule by type IB topoisomerases generates a 3′ phosphate and a 5′ hydroxyl at the cleavage site, with the type IB topoisomerase covalently binding to the 3′ terminus of a cleaved strand. As disclosed herein, type I and type II topoisomerases, as well as catalytic domains and mutant forms thereof, are useful for generating double stranded recombinant nucleic acid molecules covalently linked in both strands according to a method of the invention.

Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson, et al., J. Biol. Chem. 275:1533-1540, 2000; Hanai, et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang, et al., J. Biol. Chem. 270:23700-23705, 1995, which is incorporated herein by reference). A homolog, the traE protein of plasmid RP4, has been described by Li, et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Type IB topoisomerases include the nuclear type I topoisomerases present in all eukaryotic cells and those encoded by vaccinia and other cellular poxviruses (see Cheng, et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B,:271-297, 1994; Gupta, et al., Biochim. Biophys. Acta 1262:1-14, 1995, each of which is incorporated herein by reference; see, also, Berger, supra, 1998). Viral type IB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen, et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCT/US95/16099; PCT/US98/12372, each of which is incorporated herein by reference; see, also, Cheng, et al., supra, 1998).

Type II topoisomerases include, for example, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998;). Like the type IB topoisomerases, the type II topoisomerases have both cleaving and ligating activities. In addition, like type IB topoisomerase, substrate nucleic acid molecules can be prepared such that the type II topoisomerase can form a covalent linkage to one strand at a cleavage site. For example, calf thymus type II topoisomerase can cleave a substrate nucleic acid molecule containing a 5′ recessed topoisomerase recognition site positioned three nucleotides from the 5′ end, resulting in dissociation of the three nucleotide sequence 5′ to the cleavage site and covalent binding the of the topoisomerase to the 5′ terminus of the nucleic acid molecule (Andersen, et al., supra, 1991). Furthermore, upon contacting such a type II topoisomerase charged nucleic acid molecule with a second nucleotide sequence containing a 3′ hydroxyl group, the type II topoisomerase can ligate the sequences together, and then is released from the recombinant nucleic acid molecule. As such, type II topoisomerases also are useful for performing methods of the invention.

The various topoisomerases exhibit a range of sequence specificity. For example, type II topoisomerases can bind to a variety of sequences, but cleave at a highly specific recognition site (see Andersen, et al., J. Biol. Chem. 266:9203-9210, 1991, which is incorporated herein by reference.). In comparison, the type IB topoisomerases include site specific topoisomerases, which bind to and cleave a specific nucleotide sequence (“topoisomerase recognition site”). Upon cleavage of a nucleic acid molecule by a topoisomerase, for example, a type IB topoisomerase, the energy of the phosphodiester bond is conserved via the formation of a phosphotyrosyl linkage between a specific tyrosine residue in the topoisomerase and the 3′ nucleotide of the topoisomerase recognition site. Where the topoisomerase cleavage site is near the 3′ terminus of the nucleic acid molecule, the downstream sequence (3′ to the cleavage site) can dissociate, leaving a nucleic acid molecule having the topoisomerase covalently bound to the newly generated 3′ end.

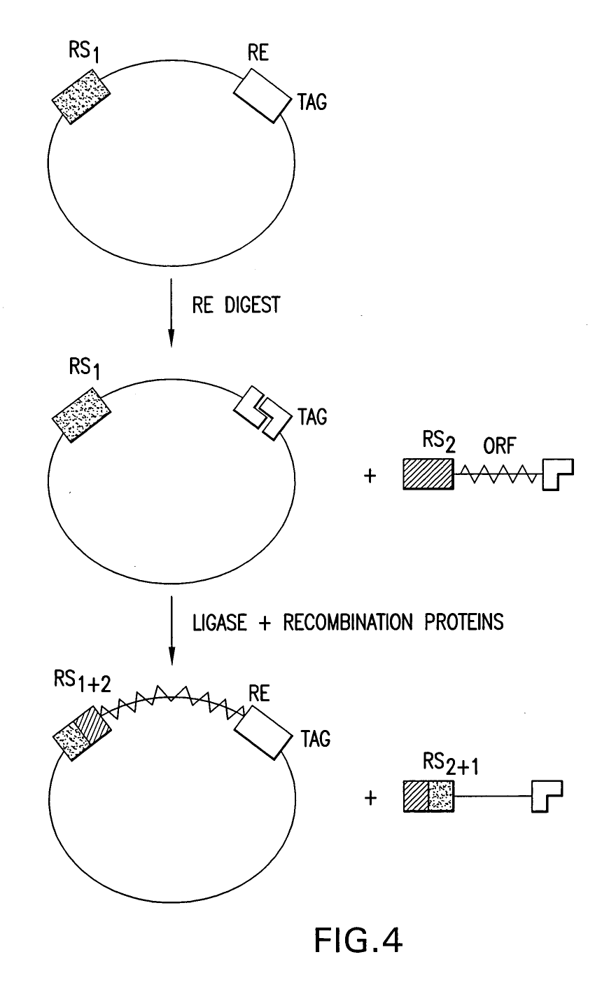

With reference to FIG. 4, a combination of restriction digestion/ligation and recombinational cloning may be used to construct nucleic acid molecules of the invention. A nucleic acid molecule (e.g., a plasmid) having at least one recognition site (e.g., recombination site) (RSI) and at least one restriction enzyme site (RE) may be constructed. A molecule of this type may comprise a tag sequence, optionally located adjacent to the restriction enzyme site. The molecule may be digested with a restriction enzyme resulting in a linear molecule. The resultant linear molecule may be contacted with a second nucleic acid molecule comprising at least one recombination site and having an end compatible with the restriction digested end of the linear first nucleic acid molecule. In the presence of ligase and the appropriate recombination proteins, the second nucleic acid molecule is covalently coupled to the first nucleic acid molecule replacing the portion of the first nucleic acid molecule between the recombination site and the restriction enzyme site. Those skilled in the art will appreciate that one or more topoisomerases may be used in place of or in combination with the restriction enzyme digestion and/or ligation reactions. Thus, the invention contemplates linear molecules, which may be charged at one end with one or more topoisomerases, containing at least one recombination site. The invention also contemplates compositions comprising such molecules, reaction mixtures comprising such molecules, and methods of making and using such molecules.

Suppressor tRNAs

Mutant tRNA molecules that recognize what are ordinarily stop codons suppress the termination of translation of an mRNA molecule and are termed suppressor tRNAs. Three codons are used by both eukaryotes and prokaryotes to signal the end of gene. When transcribed into MRNA, the codons have the following sequences: UAG (amber), UGA (opal) and UAA (ochre). Under most circumstances, the cell does not contain any tRNA molecules that recognize these codons. Thus, when a ribosome translating an mRNA reaches one of these codons, the ribosome stalls and falls of the RNA, terminating translation of the mRNA. The release of the ribosome from the mRNA is mediated by specific factors (see S. Mottagui-Tabar, Nucleic Acids Research 26(11), 2789, 1998). A gene with an in-frame stop codon (TAA, TAG, or TGA) will ordinarily encode a protein with a native carboxy terminus. However, suppressor tRNAs can result in the insertion of amino acids and continuation of translation past stop codons.

A number of such suppressor tRNAs have been found. Examples include, but are not limited to, the supE, supP, supD, supF and supZ suppressors, which suppress the termination of translation of the amber stop codon, supB, glT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. For a more detailed discussion of suppressor tRNAs, the reader may consult Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.

Mutations that enhance the efficiency of termination suppressors, i.e., increase the read through of the stop codon, have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Under ordinary circumstances, host cells would not be expected to be healthy if suppression of stop codons is too efficient. This is because of the thousands or tens of thousands of genes in a genome, a significant fraction will naturally have one of the three stop codons; complete read-through of these would result in a large number of aberrant proteins containing additional amino acids at their carboxy termini. If some level of suppressing tRNA is present, there is a race between the incorporation of the amino acid and the release of the ribosome. Higher levels of tRNA may lead to more read-through although other factors, such as the codon context, can influence the efficiency of suppression.

Organisms ordinarily have multiple genes for tRNAs. Combined with the redundancy of the genetic code (multiple codons for many of the amino acids), mutation of one tRNA gene to a suppressor tRNA status does not lead to high levels of suppression. The TAA stop codon is the strongest, and most difficult to suppress. The TGA is the weakest, and naturally (in E. coli) leaks to the extent of 3%. The TAG (amber) codon is relatively tight, with a read-through of ˜1% without suppression. In addition, the amber codon can be suppressed with efficiencies on the order of 50% with naturally occurring suppressor mutants. Suppression in some organisms (e.g., E. coli) may be enhanced when the nucleotide following the stop codon is an adenosine. Thus, the present invention contemplates nucleic acid molecules having a stop codon followed by an adenosine (e.g., having the sequence TAGA, TAAA, and/or TGAA).

Suppression has been studied for decades in bacteria and bacteriophages. In addition, suppression is known in yeast, flies, plants and other eukaryotic cells including mammalian cells. For example, Capone, et al. (Molecular and Cellular Biology 6(9):3059-3067, 1986) demonstrated that suppressor tRNAs derived from mammalian tRNAs could be used to suppress a stop codon in mammalian cells. A copy of the E. coli chloramphenicol acetyltransferase (cat) gene having a stop codon in place of the codon for serine 27 was transfected into mammalian cells along with a gene encoding a human serine tRNA that had been mutated to form an amber, ochre, or opal suppressor derivative of the gene. Successful expression of the cat gene was observed. An inducible mammalian amber suppressor has been used to suppress a mutation in the replicase gene of polio virus and cell lines expressing the suppressor were successfully used to propagate the mutated virus (Sedivy, et al., Cell 50: 379-389 (1987)). The context effects on the efficiency of suppression of stop codons by suppressor tRNAs has been shown to be different in mammalian cells as compared to E. coli (Phillips-Jones, et al., Molecular and Cellular Biology 15(12): 6593-6600 (1995), Martin, et al., Biochemical Society Transactions 21: (1993)) Since some human diseases are caused by nonsense mutations in essential genes, the potential of suppression for gene therapy has long been recognized (see Temple, et al., Nature 296(5857):537-40 (1982)). The suppression of single and double nonsense mutations introduced into the diphtheria toxin A-gene has been used as the basis of a binary system for toxin gene therapy (Robinson, et al., Human Gene Therapy 6:137-143 (1995)).

Use of Suppressor tRNAs to Conditionally Express Fusion Proteins

Because the methods used to create the nucleic acids of the invention are site specific, the orientation and/or reading frame of a nucleic acid sequence on a first nucleic acid molecule can be controlled with respect to the orientation and/or reading frame of a sequence on a second nucleic acid molecule when all or a portion of the molecules are joined in a recombination and/or topoisomerase-mediated reaction. This control makes the construction of fusions between sequences present on different nucleic acid molecules a simple matter.

In general terms, an open reading frame may be expressed in four forms: native at both amino and carboxy termini, modified at either end, or modified at both ends. A nucleic acid sequence of interest comprising an ORF of interest may include the N-terminal methionine ATG codon, and a stop codon at the carboxy end, of the ORF, thus ATG-ORF-stop. Frequently, the nucleic acid molecule comprising the sequence of interest will include translation initiation sequences, tis, that may be located upstream of the ATG that allow expression of the gene, thus tis-ATG-ORF-stop. Constructs of this sort allow expression of an ORF as a protein that contains the same amino and carboxy amino acids as in the native, uncloned, protein. When such a construct is fused in-frame with an amino-terminal protein tag, e.g., GST, the tag will have its own tis, thus tis-ATG-tag-tis-ATG-ORF-stop, and the bases comprising the tis of the ORF will be translated into amino acids between the tag and the ORF. In addition, some level of translation initiation may be expected in the interior of the mRNA (i.e., at the ORF's ATG and not the tag's ATG) resulting in a certain amount of native protein expression contaminating the desired protein.

DNA (lower case): tis1-atg-tag-tis2-atg-orf-stop

RNA (lower case, italics): tis1-atg-tag-tis2-atg-orf-stop

Protein (upper case): ATG-TAG-TIS2-ATG-ORF (tis1 and stop are not translated)+contaminating ATG-ORF (translation of ORF beginning at tis2).

Using the methods disclosed herein, one skilled in the art can construct a vector containing a tag adjacent to a recombination site permitting the in frame fusion of a tag to the C- and/or N-terminus of the ORF of interest.

Given the ability to rapidly create a number of clones in a variety of vectors, there is a need in the art to maximize the number of ways a single cloned ORF can be expressed without the need to manipulate the construct itself. The present invention meets this need by providing materials and methods for the controlled expression of a C- and/or N-terminal fusion to a target ORF using one or more suppressor tRNAs to suppress the termination of translation at a stop codon. Thus, the present invention provides materials and methods in which a gene construct is prepared flanked with recombination sites.

The construct may be prepared with a sequence coding for a stop codon preferably at the C-terminus of the ORF encoding the protein of interest. In some embodiments, a stop codon can be located adjacent to the ORF, for example, within the recombination site flanking the gene or at or near the 3′ end of the sequence of interest before a recombination site. The target gene construct can be transferred through recombination to various vectors that can provide various C-terminal or N-terminal tags (e.g., GFP, GST, His Tag, GUS, etc.) to the ORF of interest. When the stop codon is located at the carboxy terminus of the ORF, expression of the ORF with a “native” carboxy end amino acid sequence occurs under non-suppressing conditions (i.e., when the suppressor tRNA is not expressed) while expression of the ORF as a carboxy fusion protein occurs under suppressing conditions. Those skilled in the art will recognize that any suppressors and any codons could be used in the practice of the present invention. Suppressors may insert any amino acid at the position corresponding to the stop codon, for example, Ala, Arg, Asn, Asp, Cys, Gln, Glu, Gly, His, Ile, Leu, Lys, Met, Phe, Pro, Ser, Thr, Trp, Tyr, or Val may be inserted. In some embodiments, serine may be inserted.

In some embodiments, the gene coding for the suppressing tRNA may be incorporated into the vector from which the target ORF is to be expressed. In other embodiments, the gene for the suppressor tRNA may be in the genome of the host cell. In still other embodiments, the gene for the suppressor may be located on a separate viral vector or other vector—i.e., plasmid—and provided in trans. In embodiments of this type, the vector containing the suppressor gene may be a recombinant adenoviral vector and cells may be co-infected with a viral vector expressing a sequence of interest and a viral vector expressing a suppressor tRNA.

More than one copy of a suppressor tRNA may be provided in all of the embodiments described herein. For example, a host cell may be provided that contains multiple copies of a gene encoding the suppressor tRNA. Alternatively, multiple gene copies of the suppressor tRNA under the same or different promoters may be provided in the same vector background as the target ORF of interest. In some embodiments, multiple copies of a suppressor tRNA may be provided in a different vector than the one containing the target ORF of interest. In other embodiments, one or more copies of the suppressor tRNA gene may be provided on the vector containing the ORF for the protein of interest and/or on another vector and/or in the genome of the host cell or in combinations of the above. When more than one copy of a suppressor tRNA gene is provided, the genes may be expressed from the same or different promoters that may be the same or different as the promoter used to express the ORF encoding the protein of interest.

In some embodiments, two or more different suppressor tRNA genes may be provided. In embodiments of this type one or more of the individual suppressors may be provided in multiple copies and the number of copies of a particular suppressor tRNA gene may be the same or different as the number of copies of another suppressor tRNA gene. Each suppressor tRNA gene, independently of any other suppressor tRNA gene, may be provided on the vector used to express the ORF of interest and/or on a different vector and/or in the genome of the host cell. A given tRNA gene may be provided in more than one place in some embodiments. For example, a copy of the suppressor tRNA may be provided on the vector containing the ORF of interest while one or more additional copies may be provided on an additional vector and/or in the genome of the host cell. When more than one copy of a suppressor tRNA gene is provided, the genes may be expressed from the same or different promoters that may be the same or different as the promoter used to express the ORF encoding the protein of interest and may be the same or different as a promoter used to express a different tRNA gene.

In some embodiments of the present invention, the target ORF of interest and the gene expressing the suppressor tRNA may be controlled by the same promoter. In other embodiments, the target ORF of interest may be expressed from a different promoter than the suppressor tRNA. Those skilled in the art will appreciate that, under certain circumstances, it may be desirable to control the expression of the suppressor tRNA and/or the target ORF of interest using a regulatable promoter. For example, either the target ORF of interest and/or the gene expressing the suppressor tRNA may be controlled by a promoter such as the lac promoter or derivatives thereof such as the tac promoter. In some embodiments, both the target ORF of interest and the suppressor tRNA gene are expressed from the T7 RNA polymerase promoter and, optionally, are expressed as part of one RNA molecule. In embodiments of this type, the portion of the RNA corresponding to the suppressor tRNA is processed from the originally transcribed RNA molecule by cellular factors.

In some embodiments, the expression of the suppressor tRNA gene may be under the control of a different promoter from that of the ORF of interest. In some embodiments, it may be possible to express the suppressor gene before the expression of the target ORF. This would allow levels of suppressor to build up to a high level, before they are needed to allow expression of a fusion protein by suppression of a the stop codon. For example, in embodiments of the invention where the suppressor gene is controlled by a promoter inducible with IPTG, the target ORF is controlled by the T7 RNA polymerase promoter and the expression of the T7 RNA polymerase is controlled by a promoter inducible with an inducing signal other than IPTG, e.g., NaCl, one could turn on expression of the suppressor tRNA gene with IPTG prior to the induction of the T7 RNA polymerase gene and subsequent expression of the ORF of interest. In some embodiments, the expression of the suppressor tRNA might be induced about 15 minutes to about one hour before the induction of the T7 RNA polymerase gene. In one embodiment, the expression of the suppressor tRNA may be induced from about 15 minutes to about 30 minutes before induction of the T7 RNA polymerase gene. In some embodiments, the expression of the T7 RNA polymerase gene is under the control of an inducible promoter.

In additional embodiments, the expression of the target ORF of interest and the suppressor tRNA can be arranged in the form of a feedback loop. For example, the target ORF of interest may be placed under the control of the T7 RNA polymerase promoter while the suppressor gene is under the control of both the T7 promoter and the lac promoter. The T7 RNA polymerase gene itself is also under the control of both the T7 promoter and the lac promoter. In addition, the T7 RNA polymerase gene has an amber stop mutation replacing a normal tyrosine codon, e.g., the 28th codon (out of 883). No active T7 RNA polymerase can be made before levels of suppressor are high enough to give significant suppression. Then expression of the polymerase rapidly rises, because the T7 polymerase expresses the suppressor gene as well as itself. In other preferred embodiments, only the suppressor gene is expressed from the T7 RNA polymerase promoter. Embodiments of this type would give a high level of suppressor without producing an excess amount of T7 RNA polymerase. In other preferred embodiments, the T7 RNA polymerase gene has more than one amber stop mutation. This will require higher levels of suppressor before active T7 RNA polymerase is produced.

In some embodiments of the present invention it may be desirable to have more than one stop codon suppressible by more than one suppressor tRNA. A recombinant viral vector may be constructed so as to permit the regulatable expression of N- and/or C-terminal fusions of a protein of interest from the same construct. A viral vector may comprise a first tag sequence expressed from a promoter and may include a first stop codon in the same reading frame as the tag. The stop codon may be located anywhere in the tag sequence and is preferably located at or near the C-terminal of the tag sequence. The stop codon may also be located in a recombination site or in an internal ribosome entry sequence (IRES). The viral vector may also include a sequence of interest preferably comprising a ORF of interest that includes a second stop codon. The first tag and the ORF of interest are preferably in the same reading frame although inclusion of a sequence that causes frame shifting to bring the first tag into the same reading frame as the ORF of interest is within the scope of the present invention. The second stop codon is preferably in the same reading frame as the ORF of interest and is preferably located at or near the end of the coding sequence for the ORF. The second stop codon may optionally be located within a recombination site located 3′ to the sequence of interest. The construct may also include a second tag sequence in the same reading frame as the ORF of interest and the second tag sequence may optionally include a third stop codon in the same reading frame as the second tag. A transcription terminator and/or a polyadenylation sequence may be included in the construct after the coding sequence of the second tag. The first, second and third stop codons may be the same or different. In some embodiments, all three stop codons are different. In embodiments where the first and the second stop codons are different, the same construct may be used to express an N-terminal fusion, a C-terminal fusion and the native protein by varying the expression of the appropriate suppressor tRNA. For example, to express the native protein, no suppressor tRNAs are expressed and protein translation is controlled by an appropriately located IRES. When an N-terminal fusion is desired, a suppressor tRNA that suppresses the first stop codon is expressed while a suppressor tRNA that suppresses the second stop codon is expressed in order to produce a C-terminal fusion. In some instances it may be desirable to express a doubly tagged protein of interest in which case suppressor tRNAs that suppress both the first and the second stop codons may be expressed.

Construction and Uses Nucleic Acid Molecules of the Invention.

As discussed below in more detail, in one aspect, the invention provides a modular system for constructing viruses, e.g., viral vectors, having particular functions or activities. The present invention also includes methods for preparing viruses, e.g., viral vectors, containing more than one nucleic acid insert (e.g., two, three, four, five, six, eight, ten, twelve, fifteen, twenty, thirty, forty, fifty, etc. inserts). In one general embodiment of the invention, viral vectors and/or nucleic acids molecules of the invention are prepared as follows. Nucleic acid molecules that are to ultimately be incorporated into the viral vector are obtained (e.g., purchased, prepared by PCR or by the preparation of cDNA using reverse transcriptase). Suitable recombination sites are either incorporated into the 5′ and/or 3′ ends of the nucleic acid molecules during synthesis or added later. A nucleic acid comprising all or a portion of a viral genome and the nucleic acid to be incorporated are combined in the presence of one or more recombination proteins in order to construct the desired viral vector.

In some embodiments of the invention nucleic acid molecules of the invention may be combined using various combinations of techniques known in the art. When a first nucleic acid molecule is to be joined with a second nucleic acid molecule, the ends of the molecules may be joined using the same or different techniques. For example, one end of a first nucleic acid molecule to be joined with a second nucleic acid molecule may comprise one type of recognition site (e.g., a topoisomerase site) and the other end may comprise a different type of site (e.g., a recombination site or a restriction enzyme site). In various embodiments, a nucleic acid molecule may have a restriction enzyme site on one end and a topoisomerase site on the other end, a restriction enzyme site on one end and a recombination site on the other end, or a topoisomerase site on one end and a recombination site on the other end. Those skilled in the art will appreciate that a ligase and/or topoisomerase may be used to link an end having a restriction site with another nucleic acid molecule. When topoisomerase is used to join two nucleic acid molecules, either or both strands may be covalently joined. FIG. 3 shows examples of the covalent joining of both strands.

To construct a modular viral vector, one or more nucleic acid segments comprising one or more recombination sites and also comprising a viral sequence may be prepared. In some embodiments, multiple segments, each having at least one recombination site and some having viral sequences (e.g., baculoviral or adenoviral sequences) may be constructed and combined to produce a nucleic acid molecule of the invention. For example, a nucleic acid segment comprising an adenoviral ITR and a recombination site may be prepared. Further, a plurality of nucleic acid segments, each comprising a different portion of the adenoviral genome flanked by recombination sites, may be prepared. In some embodiments, the entire genome of an adenovirus is prepared in segments flanked by recombination sites. Such segments may be combined with one or more additional segments comprising additional sequences of interest such that, after combining, a nucleic acid comprising all or a portion of an adenoviral genome and comprising a sequence of interest is formed.

Segments of an adenoviral genome may be prepared from different serotypes of adenovirus, for example, Ad5, Ad3, Ad10, etc., and viral vectors having a mixed serotype, (e.g., some determinants of Ad5 and some of Ad10) may be prepared. It may be desirable to vary the most immunogenic portions of the viruses in situations where multiple administrations of viral vectors are contemplated.

Each segment of the adenoviral genome may comprise one or more regions of the genome, for example, left ITR, right ITR, packaging signal, E1, E2, E3, E4, and/or one or more late regions. In some embodiments, a segment may comprise the entire adenoviral genome except one region that is on a different segment. For example, an entire adenoviral genome except for the packaging signal may be prepared on one segment and the packaging signal may be prepared on a different segment. The two segments may be combined (e.g., using recombinational cloning) to produce a viral vector of the invention. Likewise, an entire adenoviral genome may be prepared that lacks one or more of the following elements: left ITR, E1, E2, E3, E4, or right ITR. The lacking element may be prepared on a separate segment and the two segments may be combined to produce a viral vector. One or more sequences of interest may be incorporated into either segment prior to combining the segments in order to produce an adenoviral vector containing one or more sequences of interest. More than one viral region may be prepared on a segment, for example, the left ITR, packaging signal, and E3 region may be prepared on one segment with the remainder of the adenoviral functions necessary to prepare a viral vector present on one or more other segments. Sequences of interest may be present on any one of the segments.

Typically, the nucleic acid molecules may be dissolved in an aqueous buffer and added to the reaction mixture. One suitable set of conditions is 4 μl CLONASE™ enzyme mixture (e.g., Invitrogen Corporation, Cat. Nos. 11791-019 and 11789-013), 4 μl 5× reaction buffer and nucleic acid and water to a final volume of 20 μl. This will typically result in the inclusion of about 200 ng of Int and about 80 ng of IHF in a 20 μl BP reaction and about 150 ng Int, about 25 ng IHF and about 30 ng Xis in a 20 μl LR reaction.

Proteins for conducting an LR reaction may be stored in a suitable buffer, for example, LR Storage Buffer, which may comprise about 50 mM Tris at about pH 7.5, about 50 mM NaCl, about 0.25 mM EDTA, about 2.5 mM Spermidine, and about 0.2 mg/ml BSA. When stored, proteins for an LR reaction may be stored at a concentration of about 37.5 ng/μl INT, 10 ng/μl IHF and 15 ng/μl XIS. Proteins for conducting a BP reaction may be stored in a suitable buffer, for example, BP Storage Buffer, which may comprise about 25 mM Tris at about pH 7.5, about 22 mM NaCl, about 5 mM EDTA, about 5 mM Spermidine, about 1 mg/ml BSA, and about 0.0025% Triton X-100. When stored, proteins for an BP reaction may be stored at a concentration of about 37.5 ng/μl INT and 20 ng/μl IHF. One skilled in the art will recognize that enzymatic activity may vary in different preparations of enzymes. The amounts suggested above may be modified to adjust for the amount of activity in any specific preparation of enzymes.

A suitable 5×reaction buffer for conducting recombination reactions may comprise 100 mM Tris pH 7.5, 88 mM NaCl, 20 mM EDTA, 20 mM Spermidine, and 4 mg/ml BSA. Thus, in a recombination reaction, the final buffer concentrations may be 20 mM Tris pH 7.5, 17.6 mM NaCl, 4 mM EDTA, 4 mM Spermidine, and 0.8 mg/ml BSA. Those skilled in the art will appreciate that the final reaction mixture may incorporate additional components added with the reagents used to prepare the mixture, for example, a BP reaction may include 0.005% Triton X-100 incorporated from the BP Clonase™.

In some preferred embodiments, particularly those in which attL sites are to be recombined with attR sites, the final reaction mixture may include about 50 mM Tris HCl, pH 7.5, about 1 mM EDTA, about 1 mg/ml BSA, about 75 mM NaCl and about 7.5 mM spermidine in addition to recombination enzymes and the nucleic acids to be combined. In other preferred embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, and about 5 mM spermidine.

In some preferred embodiments, particularly those in which attL sites are to be recombined with attR sites, the final reaction mixture may include about 40 mM Tris HCl, pH 7.5, about 1 mM EDTA, about 1 mg/ml BSA, about 64 mM NaCl and about 8 mM spermidine in addition to recombination enzymes and the nucleic acids to be combined. One of skill in the art will appreciate that the reaction conditions may be varied somewhat without departing from the invention. For example, the pH of the reaction may be varied from about 7.0 to about 8.0; the concentration of buffer may be varied from about 25 mM to about 100 mM; the concentration of EDTA may be varied from about 0.5 mM to about 2 mM; the concentration of NaCl may be varied from about 25 mM to about 150 mM; and the concentration of BSA may be varied from 0.5 mg/ml to about 5 mg/ml. In other preferred embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, about 5 mM spermidine and about 0.005% detergent (e.g., Triton X-100).

The invention also includes viral vectors, in addition to adenoviral vectors (e.g., baculoviral vectors), which contain either all or, part of one or more viral genome. Using vectors comprising baculoviral nucleic acid for purposes of illustration, vectors of the invention include those which comprise one or more element (e.g., one or more functional element) of a baculoviral genome, as well as vectors which comprise one or more element (e.g., promoters, transcription terminators, polyA signals or sequences, ribosome binding sites, enhancers, ORFs or portions thereof, etc.) of one or more other viral genomes. Typically, these vectors will include one or more recombination site, as described elsewhere herein.

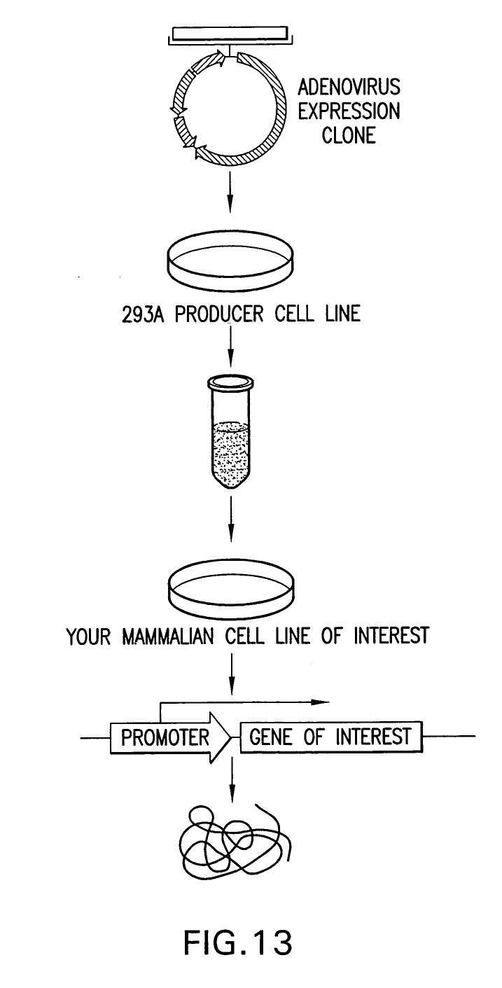

One specific example of a vector of the invention is shown schematically in FIG. 13. This vector contains three separate baculoviral elements. More specifically, the vector shown in FIG. 13 comprises the IE2 gene promoter and IE2 gene polyA region of Orgyia pseudotsugata. The vector also includes the GP64 promoter of Autographa californica. Thus, nucleic acid molecules of the invention include vectors which contain one or more elements (e.g., an element described herein) derived from one or more viral genome (e.g., adenoviral genome, baculoviral genome, etc.). Further, these elements may be from the same or different viruses.

The invention further includes nucleic acid molecules which comprise modified elements of viral genomes. These modified elements may be defined and/or described within the scope of the invention in any number of ways. Examples of such ways include (1) function (e.g., a property conferred upon a nucleic acid which contains the element), (2) % sequence identity, and (3) % homology or sequence identity of expression products, as well as combinations of these ways. Percent homology or sequence identity will typically be determined with reference to the nucleotide or amino acid sequence of another nucleic acid or polypeptide.

As indicated above, viral elements and modified viral elements suitable for use with the invention may be described by their ability to confer one or more functional properties on nucleic acid molecules which contain them. Using the GP64 promoter as an example, this promoter is an inducible promoter which exhibits low level basal constitutive activity. In other words, in the absence of induction, the GP64 promoter allows for low level of transcription when operably linked to a nucleic acid segment. Functional properties are also associated with other viral elements, such as origins of replication, polyA tail sequences, packaging signals, LTRs, etc. Further, depending on the particular element, functional activity can be assessed at either the level of the vector (e.g., the DNA or RNA level), a transcription product (e.g., the RNA level), and/or a translation product (e.g., the polypeptide level). Thus, the invention further includes nucleic acid molecules which comprise modified viral elements which retain all or some of the functions of the viral elements from which they are derived (e.g., the “wild-type” viral element). In many instances, a modified element will retain at least one functional property of the element from which they are derived. In particular embodiments, the modified element will (1) have at least one additional property not associated with the element from which it was derived, (2) be deficient in at least one property associated with the element from which it was derived, and/or (3) have increased or decreased activity with respect to at least one property associated with the element from which it was derived.

As also indicated above, modified elements (e.g., modified viral elements) contained in nucleic acid molecules of the invention may be described by their structural similarity to elements from which they are derived. For example, modified elements may be at least 50% identical, at least 55% identical, at least 60% identical, at least 65% identical, at least 70% identical, at least 75% identical, at least 80% identical, at least 85% identical, at least 90% identical, or at least 95% identical at the nucleic acid level to the nucleic acid molecules from which they are derived. Modified elements may also be defined by having sufficient structural similarity to the nucleic acid molecules from which they are derived (e.g., an element the nucleotide sequence of which is set out elsewhere herein) so that the two nucleic acids will hybridized. Often, these molecules will hybridized to each other under stringent hybridization conditions. In many instances, these modified elements will retain at least one property associated from the elements from which they are derived.

When modified elements of a viral genome encode a polypeptide expression product, the polypeptide may be at least 50% identical or homologous, at least 55% identical or homologous, at least 60% identical or homologous, at least 65% identical or homologous, at least 70% identical or homologous, at least 75% identical or homologous, at least 80% identical or homologous, at least 85% identical or homologous, at least 90% identical or homologous, or at least 95% identical or homologous at the amino acid level to the amino acid sequences of the polypeptide which is expressed from the nucleic acid from which the modified elements is derived. Typically, polypeptide expression products of modified elements will retain at least one functional property of polypeptides which are expressed from nucleic acids from which the modified elements are derived. In particular embodiments, the polypeptide expression product of a modified element will (1) have at least one additional property not associated with the polypeptide expression product from which the element from which it was derived, (2) be deficient in at least one property associated with the polypeptide expression product from which the element from which it was derived, and/or (3) have increased or decreased activity with respect to at least one property associated with the polypeptide expression product from which the element from which it was derived.

One example of a vector of the invention is a vector which contains the GP64 promoter of Autographa californica operably linked to a heterologous nucleic acid. In particular embodiments, the GP64 promoter has all or part of the nucleotide sequence set out in Table 12 beginning at nucleotide 3364. The invention further include nucleic acid molecules which comprise modified forms of the GP64 promoter. These modified forms of the GP64 promoter include deleted forms of the promoter which comprise at least 30 nucleotides, at least 40 nucleotides, at least 50 nucleotides, at least 60 nucleotides, at least 70 nucleotides, at least 80 nucleotides, at least 90 nucleotides, or at least 95 nucleotides.

As indicated above, vectors of the invention may comprise all or part of a viral genome. For example, vectors of the invention may comprise at least about 5%, at least about 10%, at least about 20%, at least about 30%, at least about 40%, at least about 50%, at least about 60%, at least about 70%, at least about 80%, at least about 90%, at least about 95%, or 100% of a viral genome used to prepare the vector. For example, a baculoviral vector which contains about 50% of the used to prepare it may contain about 66 kb of baculoviral nucleic acid.

It is not necessary that all viral functions required for replication be contained on a segment and be included in the final nucleic acid molecule comprising all or a portion of the viral genome. One or more required functions may be provided in trans. For example, a required function may be incorporated into the genome of a cell line and still provide the function.

Viruses lacking the function could be prepared in the cell line expressing the function. These viruses could only replicate in the cell line expressing the function and, thus, would be replication-deficient in any other cell line. Any required function could be used in this fashion, for example, the adenovirus E2 and/or E4 functions (see, Weinberg, et al., Proc. Ntl. Acad. Sci. USA 80:5383, 5386, 1983).

Segments prepared as above may be linear fragments (e.g., PCR fragments) or segments may be part of larger nucleic acid molecule (e.g., a plasmid). The segments may be combined to form a viral vector of the invention. When the segments are combined, the resultant adenoviral vector may be a linear molecule, for example, by combining linear segments using recombination cloning. A linear viral vector may be introduced (e.g., by transfection, electroporation, etc.) into an appropriate host cell and packaged virus may be isolated as described elsewhere herein. Alternatively, a viral vector may be prepared as part of a circular molecule (e.g., a plasmid) and the viral vector may released from the circular molecule (e.g., by restriction digest) and introduced into an appropriate host cell and packaged virus isolated.

When one seeks to prepare or construct a viral vector containing multiple nucleic acid inserts, these inserts can be inserted into a viral vector in either one reaction mixture or a series of reaction mixtures. For example, multiple nucleic acid segments can be linked end to end and inserted into a viral vector using reactions performed, for example, in a single reaction mixture. The nucleic acid segments in this reaction mixture can be designed so that recombination sites on their 5′ and 3′ ends result in their insertion into a nucleic acid comprising all or a portion of a viral genome in a specific order and a specific 5′ to 3′ orientation. Alternatively, nucleic acid segments can be designed so that they are inserted into a nucleic acid comprising all or a portion of a viral genome without regard to order, orientation (i.e., 5′ to 3′ orientation), the number of inserts, and/or the number of duplicate inserts.

Methods of the invention can also be used to prepare viral vectors that, upon expression of a sequence of interest contained in the viral vector, produce one or more polypeptides having one or more desired property, function, or activity (e.g., an enzymatic activity, the ability to bind a nucleic acid, etc.). For example, a polypeptide having one or more enzymatic activities might be expressed from the viral vectors of the present invention. Viral vectors of this type might be used, for example, in a gene therapy protocol to replace a missing enzymatic activity. Polypeptides produced from the viral vectors of the present invention may have other desirable characteristics, for example, a polypeptide may comprise one or more antigenic determinants. Expression of such a polypeptide may result in an immune response specific for the expressed polypeptide. Such a viral vector may be used, for example, as an immunotherapeutic, for example, a vaccine.

Methods of the invention can also be used to prepare viral vectors that, upon expression of a sequence of interest contained in the viral vector, produce one or more un-translated RNA molecules, for example, ribozymes, antisense molecules, RNAi and the like. Such a viral vector might be used, for example, to modulate (e.g., inhibit) the expression of one more RNA or polypeptide molecules produced by a host organism. Such a vector might be used, for example, to inhibit the expression of a disease associated RNA or polypeptide.

Methods of the invention can also be used to prepare viral vectors that, upon expression of a sequence of interest contained in the viral vector, produce fusion proteins having more than one property, function, or activity. Further, the expression product can be produced in such a manner as to facilitate its export from the cell. For example, these expression products can be fusion proteins that contain a signal peptide that results in export of the protein from the cell. One application where cell export may be desirable is where the proteins that are to be exported are enzymes that interact with extracellular substrates.

In a specific embodiment, the invention further provides methods for introducing viral vectors and/or nucleic acids molecules of the invention into animals (e.g., humans) and animal cells (e.g., human cells), as part of a gene therapy protocol. Viral vectors of the present invention may be designed such that compositions comprising the vectors are free of viral vectors that are replication competent in the target cell. Thus, in some embodiments, viral vectors of the present invention are replication restricted, i.e., can replicate in a permissive cell type, e.g., 293 cells, and cannot replicate in a target cell type, e.g., patient cells.

Gene therapy refers to therapy performed by the administration to a subject of an expressed or expressible nucleic acid molecule. In many embodiments of the invention, nucleic acid molecules of the invention will encoded one or more proteins (e.g., one or more fusion proteins) that mediate at least one therapeutic effect. Thus, the invention provide nucleic acid molecules and methods for use in gene therapy.

Viral vectors and/or nucleic acids molecules of the invention can be used to prepare gene therapy vectors designed to replace genes that reside in the genome of a cell, to delete such genes, or to insert a heterologous gene or groups of genes. When viral vectors and/or nucleic acids molecules of the invention function to delete or replace a gene or genes, the gene or genes being deleted or replaced may lead to the expression of either a “normal” phenotype or an aberrant phenotype. One example of an aberrant phenotype is the disease cystic fibrosis. Further, the gene therapy vectors may be either stably maintained (e.g., integrate into cellular nucleic acid by homologous or site specific recombination) or non-stably maintained in cells.

Further, viral vectors and/or nucleic acids molecules of the invention may be used to suppress “abnormal” phenotypes or complement or supplement “normal” phenotypes that result from the expression of endogenous genes. One example of a viral vector of the invention designed to suppress an abnormal phenotype would be where an expression product of the viral vector has dominant/negative activity. An example of a viral vector of the invention designed to supplement a normal phenotype would be where introduction of the viral vector effectively results in the amplification of a gene resident in the cell.

In some embodiments, viral vectors and/or nucleic acids of the present invention may be used to prevent or inhibit the expression of one or more genes in an organism, for example, by homology-dependent gene silencing (HDGS, see, for example, Bernstein, et al., RNA 7:1509-21 (2001), and Bass, Cell 101:235-238 (2000)). The genes expression of which is to be inhibited, i.e., silenced, may be endogenous to the organism or may be exogenous to the organism.

Viral vectors and/or nucleic acid molecules of the invention may be prepared to generate interfering RNAs (RNAi). RNAi is double-stranded RNA that results in degradation of specific mRNAs, and can also be used to lower or eliminate gene expression. Viral vectors and/or nucleic acid molecules of the invention may be engineered, for example, to produce dsRNA molecules by, for example, engineering the viral vectors and/or nucleic acid molecules to have a sequence that, when transcribed, folds back upon itself to generate a hairpin molecule containing a double-stranded portion. One strand of the double-stranded portion may correspond to all or a portion of the sense strand of the mRNA transcribed from the gene to be silenced while the other strand of the double-stranded portion may correspond to all or a portion of the antisense strand. Other methods of producing a double-stranded RNA molecule may be used, for example, a viral vector and/or nucleic acid molecules may be engineered to have a first sequence that, when transcribed, corresponds to all or a portion of the sense strand of the MRNA transcribed from the gene to be silenced and a second sequence that, when transcribed, corresponds to all or portion of an antisense strand (i.e., the reverse complement) of the mRNA transcribed from the gene to be silenced. This may be accomplished by putting the first and the second sequence on the same strand of the viral vector each under the control of its own promoter. Alternatively, two promoters may be positioned on opposite strands of the viral vector such that expression from each promoter results in transcription of one strand of the double-stranded RNA. In some embodiments, it may be desirable to have the first sequence on one viral vector or nucleic acid molecule and the second sequence on a second viral vector or nucleic acid molecule and to introduce both vectors or molecules into a cell containing the gene to be silenced. In other embodiments, a viral vector or nucleic acid molecule containing only the antisense strand may be introduced and the mRNA transcribed from the gene to be silenced may serve as the other strand of the double-stranded RNA. In some embodiments, a dsRNA to be used to silence a gene may have one or more regions of homology to a gene to be silenced. Regions of homology may be from about 20 bp to about 5 kbp in length, 20 bp to about 4 kbp in length, 20 bp to about 3 kbp in length, 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, from about 20 bp to about 90 bp in length, from about 20 bp to about 80 bp in length, from about 20 bp to about 70 bp in length, from about 20 bp to about 60 bp in length, from about 20 bp to about 50 bp in length, from about 20 bp to about 40 bp in length, from about 20 bp to about 30 bp in length, from about 20 bp to about 25 bp in length, from about 15 bp to about 25 bp in length, from about 17 bp to about 25 bp in length, from about 19 bp to about 25 bp in length, from about 15 bp to about 23 bp, from about 17 bp to about 23 bp, from about 19 bp to about 23 bp in length, from about 15 bp to about 21 bp, from about 17 bp to about 21 bp, or from about 19 bp to about 21 bp in length.

As discussed above, a hairpin containing molecule having a double-stranded region may be used as RNAi. The length of the double stranded region may be from about 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, 20 bp to about 90 bp in length, 20 bp to about 80 bp in length, 20 bp to about 70 bp in length, 20 bp to about 60 bp in length, 20 bp to about 50 bp in length, 20 bp to about 40 bp in length, 20 bp to about 30 bp in length, from about 20 bp to about 25 bp in length, from about 15 bp to about 25 bp in length, from about 17 bp to about 25 bp in length, from about 19 bp to about 25 bp in length, from about 15 bp to about 23 bp, from about 17 bp to about 23 bp, from about 19 bp to about 23 bp in length, from about 15 bp to about 21 bp, from about 17 bp to about 21 bp, or from about 19 bp to about 21 bp in length. The non-base-paired portion of the hairpin (i.e., loop) can be of any length that permits the two regions of homology that make up the double-stranded portion of the hairpin to fold back upon one another.

Any suitable promoter may be used to control the production of RNA from the nucleic acid molecules of the invention. Promoters may be those recognized by any polymerase enzyme. For example, promoters may be promoters for RNA polymerase II or RNA polymerase III (e.g., a U6 promoter, an H1 promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

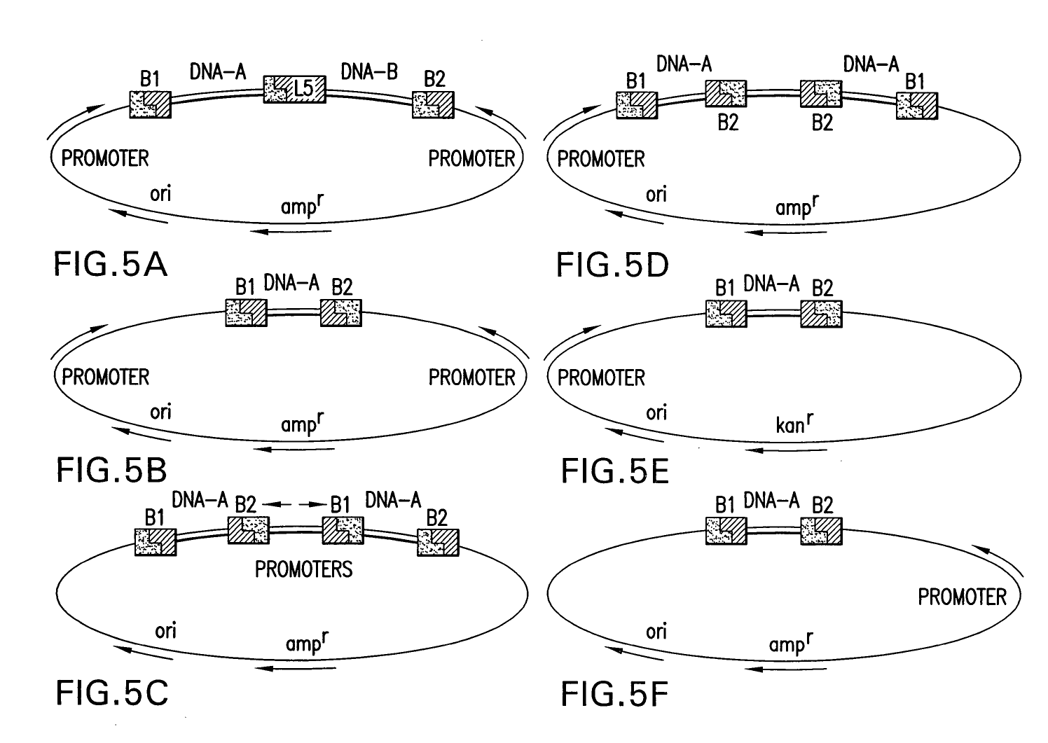

One example of a construct designed to produce RNAi is shown in FIG. 5B. In this construct, a DNA segment is inserted into a vector such that RNA corresponding to both strands are produced as two separate transcripts. Another example of a construct designed to produce RNAi is shown in FIG. 5C. In this construct, two copies of a DNA segment are inserted into a vector such that RNA corresponding to both strands are again produced. Yet another example of a construct designed to produce RNAi is shown in FIG. 5D. In this construct, two copies of a DNA segment are inserted into a vector such that RNA corresponding to both strands are produced as a single transcript. The exemplary vector system shown in shown in FIGS. 5E and 5F comprises two vectors, each of which contain copies of the same DNA segment. Expression of one of these DNA segments results in the production of sense RNA while expression of the other results in the production of an anti-sense RNA. RNA strands produced from vectors represented in FIGS. 5B-5F will thus have complementary nucleotide sequences and will generally hybridize either to each or intramolecularly under physiological conditions.

Nucleic acid segments designed to produce RNAi, such as the vectors represented in FIGS. 5B-5F, need not correspond to the full-length gene or open reading frame. For example, when the nucleic acid segment corresponds to an ORF, the segment may only correspond to part of the ORF (e.g., 50 nucleotides at the 5′ or 3′ end of the ORF). Further, while FIGS. 5B-5F show vectors designed to produce RNAi, nucleic acid segments may also perform the same function in other forms (e.g., when inserted into the chromosome of a host cell).

Gene silencing methods involving the use of compounds such as RNAi and antisense RNA, for examples, are particularly useful for identifying gene functions. More specifically, gene silencing methods can be used to reduce or prevent the expression of one or more genes in a cell or organism. Phenotypic manifestations associated with the selective inhibition of gene functions can then be used to assign role to the “silenced” gene or genes. As an example, Chuang, et al., Proc. Natl. Acad. Sci. (USA) 97:4985-4990 (2000), have demonstrated that in vivo production of RNAi can alter gene activity in Arabidopsis thaliana. Thus, the invention provides methods for regulating expression of nucleic acid molecules in cells and tissues comprising the expression of RNAi and antisense RNA. The invention further provides methods for preparing nucleic acid molecules which can be used to produce RNA corresponding to one or both strands of a DNA molecule.

Further, viral vectors and/or nucleic acids molecules of the invention may be used to insert into cells nucleic acid segments that encode expression products involved in each step of particular biological pathways (e.g., biosynthesis of amino acids such as lysine, threonine, etc.) or expression products involved in one or a few steps of such pathways. These nucleic acid molecules can be designed to, in effect, amplify genes encoding expression products in such pathways, insert genes into cells that encode expression products involved in pathways not normally found in the cells, or to replace one or more genes involved one or more steps of particular biological pathways in cells. Thus, gene therapy vectors of the invention may contain nucleic acid that results in the production one or more products (e.g., one, two, three, four, five, eight, ten, fifteen, etc.). Such vectors, especially those that lead to the production of more than one product, will be particularly useful for the treatment of diseases and/or conditions that result from the expression and/or lack of expression of more than one gene or for the treatment of more than one diseases and/or conditions.

Thus, in related aspects, the invention provides gene therapy vectors that express one or more expression products (e.g., one or more fusion proteins), methods for producing such vectors, methods for performing gene therapy using vectors of the invention, expression products of such vector (e.g., encoded RNA and/or proteins), and host cells that contain vectors of the invention.

For general reviews of the methods of gene therapy, see Goldspiel, et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993)). Methods commonly known in the art of recombinant DNA technology that can be used are described in Ausubel, et al. (eds.), 1993, Current Protocols in Molecular Biology, John Wiley & Sons, NY; and Kriegler, 1990, Gene Transfer and Expression, A Laboratory Manual, Stockton Press, N.Y.

Delivery of the viral vectors and/or nucleic acids molecules of the invention into a patient may be either direct, in which case the patient is directly exposed to the nucleic acids and/or viral vectors of the invention, or indirect, in which case, cells are first transfected/transduced with the nucleic acid/viral vector in vitro, then transplanted into the patient. These two approaches are known, respectively, as in vivo or ex vivo gene therapy.

In another specific embodiment, viral vectors that contain nucleic acid sequences encoding an antibody or other antigen-binding protein of the invention are used. The nucleic acid sequences encoding the antibody to be used in gene therapy are cloned into one or more viral vectors, which facilitates delivery of the gene into a patient.

Adenoviruses are examples of viruses that can be used to prepare viral vectors that can be used in gene therapy. Adenoviral vectors are especially attractive vehicles for delivering genes to respiratory epithelia and the use of such vectors are included within the scope of the invention. Adenoviruses naturally infect respiratory epithelia where they cause a mild disease. Other targets for adenovirus-based delivery systems are liver, the central nervous system, endothelial cells, and muscle. Adenoviral vectors have the advantage of being capable of infecting non-dividing cells. Kozarsky and Wilson, Current Opinion in Genetics and Development 3:499-503 (1993) present a review of adenovirus-based gene therapy. Bout, et al., Human Gene Therapy 5:3-10 (1994) demonstrated the use of adenoviral vectors to transfer genes to the respiratory epithelia of rhesus monkeys. Other instances of the use of adenoviral vectors in gene therapy can be found in Rosenfeld, et al., Science 252:431-434 (1991); Rosenfeld, et al., Cell 68:143-155 (1992); Mastrangeli, et al., J. Clin. Invest. 91:225-234 (1993); PCT Publication Nos. WO 94/12649 and WO 96/17053; U.S. Pat. No. 5,998,205; and Wang, et al., Gene Therapy 2:775-783 (1995), the disclosures of all of which are incorporated herein by reference in their entireties. In a one embodiment, adenoviral vectors are used for in vivo gene therapy.

Another approach to gene therapy involves transferring a gene to cells in tissue culture, for example, by infection with a viral vector of the present invention. The viral vector may contain a sequence encoding a therapeutic polypeptide or nucleic acid (i.e., antisense molecule) and may further include a sequence encoding a selectable marker. The cells are then placed under selection to isolate those cells that have taken up and are expressing the transferred gene. Those cells are then delivered to a patient.

In this embodiment, the viral vector is introduced into a cell prior to administration in vivo of the resulting recombinant cell. The resulting recombinant cells can be delivered to a patient by various methods known in the art. Recombinant blood cells (e.g., hematopoietic stem or progenitor cells) will generally be administered intravenously. The amount of cells envisioned for use depends on the desired effect, patient state, etc., and can be determined by one skilled in the art.

Cells into which a viral vector can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells (e.g., as obtained from bone marrow, umbilical cord blood, peripheral blood, fetal liver, etc.).

In a certain embodiment, the cell used for gene therapy is autologous to the patient.

In an embodiment in which recombinant cells are used in gene therapy, viral vectors containing nucleic acids encoding an antibody or other antigen-binding protein are introduced into the cells such that they are expressible by the cells and/or their progeny, and the recombinant cells are then administered in vivo for therapeutic effect. In a specific embodiment, stem or progenitor cells are used. Any stem and/or progenitor cells that can be isolated and maintained in vitro can potentially be used in accordance with this embodiment of the present invention (see, e.g., PCT Publication WO 94/08598, dated Apr. 28, 1994; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, viral vectors and/or nucleic acids molecules of the invention comprise nucleic acid sequences to be introduced for purposes of gene therapy under the control of an inducible promoter operably linked to the coding region, such that expression of the nucleic acid sequences is controllable by controlling the presence or absence of the appropriate inducer of transcription.

The viral vectors and/or nucleic acids molecules of the invention can also be used to produce transgenic organisms (e.g., animals). Animals of any species, including, but not limited to, mice, rats, rabbits, hamsters, guinea pigs, pigs, micro-pigs, goats, sheep, cows and non-human primates (e.g., baboons, monkeys, and chimpanzees) may be used to generate transgenic animals. Viruses capable of infecting the desired cell type are known to those skilled in the art and viral vectors based on these viruses may be used in the methods of the invention.

The present invention provides for transgenic organisms that carry the viral vectors and/or nucleic acids molecules of the invention or nucleic acid sequences provided by the viral vectors and/or nucleic acids molecules of the invention in all their cells, as well as organisms that carry these viral vectors or sequences in some, but not all, of their cells, i.e., mosaic organisms or chimeric. The viral vectors and/or nucleic acids molecules of the invention may be integrated as a single copy or as multiple copies. The viral vectors and/or nucleic acids molecules of the invention may also be selectively introduced into and activated in a particular cell type by following, for example, the teaching of Lasko, et al. (Lasko, et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the sequences of interest contained in the viral vectors and/or nucleic acids molecules of the invention be integrated into the chromosomal site of the endogenous gene, this will normally be done by gene targeting. Briefly, when such a technique is to be utilized, viral vectors containing some nucleotide sequences homologous to the endogenous gene are designed for the purpose of integrating, via homologous recombination with chromosomal sequences, into and disrupting the function of the nucleotide sequence of the endogenous gene. Viral vectors and/or nucleic acids molecules of the invention may also be selectively introduced into a particular cell type, thus inactivating the endogenous gene in only that cell type, by following, for example, the teaching of Gu, et al. (Gu, et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic organisms have been generated, the expression of the recombinant gene may be assayed utilizing standard techniques. Initial screening may be accomplished by Southern blot analysis or PCR techniques to analyze organism tissues to verify that integration of nucleic acid molecules of the invention has taken place. The level of mRNA expression of nucleic acid sequences introduced by the viral vectors and/or nucleic acids molecules of the invention in the tissues of the transgenic organisms may also be assessed using techniques including, but not limited to, Northern blot analysis of tissue samples obtained from the organism, in situ hybridization analysis, and reverse transcriptase-PCR (RT-PCR). Samples of tissue that express the inserted sequences may also be evaluated immunocytochemically or immunohistochemically using antibodies specific for the expression product of these nucleic acid molecules.

Once the founder organisms are produced, they may be bred, inbred, outbred, or crossbred to produce colonies of the particular organism. Examples of such breeding strategies include, but are not limited to: outbreeding of founder organisms with more than one integration site in order to establish separate lines; inbreeding of separate lines in order to produce compound transgenic organisms that express sequences of interest at higher levels because of the effects of additive expression of each copy of nucleic acid molecules of the invention; crossing of heterozygous transgenic organisms to produce organisms homozygous for a given integration site in order to both augment expression and eliminate the need for screening of organisms by DNA analysis; crossing of separate homozygous lines to produce compound heterozygous or homozygous lines; and breeding to place the nucleic acid molecules of the invention on a distinct background that is appropriate for an experimental model of interest.

Transgenic and “knock-out” organisms of the invention have uses that include, but are not limited to, model systems (e.g., animal model systems) useful in elaborating the biological function of expression products of sequences of interest, studying conditions and/or disorders associated with aberrant expression of expression products of sequences of interest, and in screening for compounds effective in ameliorating such conditions and/or disorders.

As one skilled in the art would recognize, in many instances when viral vectors containing sequences of interest are introduced into metazoan organisms, it will be desirable to operably link the sequences that encode expression products to tissue-specific transcriptional regulatory sequences (e.g., tissue-specific promoters) where production of the expression product is desired. Such promoters can be used to facilitate production of these expression products in desired tissues. A considerable number of tissue-specific promoters are known in the art.

Host Cells

The invention also relates to host cells comprising one or more of the viral vectors and/or nucleic acids molecules of the invention containing one or more sequences of interest (e.g., two, three, four, five, seven, ten, twelve, fifteen, twenty, thirty, fifty, etc.), particularly those viral vectors described in detail herein. Representative host cells that may be used according to this aspect of the invention include, but are not limited to, bacterial cells, yeast cells, plant cells and animal cells. Preferred bacterial host cells include Escherichia spp. cells (particularly E. coli cells and most particularly E. coli strains DH10B, Stbl2, DH5α, DB3, DB3.1 (preferably E. coli LIBRARY EFFICIENCYS® DB3.1™ Competent Cells; Invitrogen Corporation, Carlsbad, Calif.), DB4, DB5, JDP682 and ccdA-over (see U.S. application Ser. No. 09/518,188, filed Mar. 2, 2000, and U.S. provisional Application No. 60/475,004, filed Jun. 3, 2003, by Louis Leong et al., entitled “Cells Resistant to Toxic Genes and Uses Thereof,” the disclosures of which are incorporated by reference herein in their entireties); a DB3 cell (deposit number NRRL B-30097), a DB3.1 cell (deposit number NRRL B-30098), a DB4 cell (deposit number NRRL B-30106), a DB5 cell (deposit number NRRL B-30107), a JDP682 cell (deposit number NRRL B-30667), a ccdA-over cell (deposit number NRRL B-30668), or a mutant or derivative thereof; Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Preferred animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3, 293, CHO, COS, VERO, BHK and human cells). Preferred yeast host cells include Saccharomyces cerevisiae cells and Pichia pastoris cells. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.).

Nucleic acid molecules to be used in the present invention may comprise one or more origins of replication (ORIs), and/or one or more selectable markers. In some embodiments, molecules may comprise two or more ORIs at least two of which are capable of functioning in different organisms (e.g., one in prokaryotes and one in eukaryotes). For example, a nucleic acid may have an ORI that functions in one or more prokaryotes (e.g., E. coli, Bacillus, etc.) and another that functions in one or more eukaryotes (e.g., yeast, insect, mammalian cells, etc.). Selectable markers may likewise be included in nucleic acid molecules of the invention to allow selection in different organisms. For example, a nucleic acid molecule may comprise multiple selectable markers, one or more of which functions in prokaryotes and one or more of which functions in eukaryotes.

Methods for introducing the viral vectors and/or nucleic acids molecules of the invention into the host cells described herein, to produce host cells comprising one or more of the viral vectors and/or nucleic acids molecules of the invention, will be familiar to those of ordinary skill in the art. For instance, the nucleic acid molecules and/or viral vectors of the invention may be introduced into host cells using well known techniques of infection, transduction, electroporation, transfection, and transformation. The nucleic acid molecules and/or viral vectors of the invention may be introduced alone or in conjunction with other nucleic acid molecules and/or vectors and/or proteins, peptides or RNAs. Alternatively, the nucleic acid molecules and/or viral vectors of the invention may be introduced into host cells as a precipitate, such as a calcium phosphate precipitate, or in a complex with a lipid. Electroporation also may be used to introduce the nucleic acid molecules and/or viral vectors of the invention into a host. Likewise, such molecules may be introduced into chemically competent cells such as E. coli. If the vector is a virus, it may be packaged in vitro or introduced into a packaging cell and the packaged virus may be transduced into cells. Thus nucleic acid molecules of the invention may contain and/or encode one or more packaging signal (e.g., viral packaging signals that direct the packaging of viral nucleic acid molecules). Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W. H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

Kits

In another aspect, the invention provides kits that may be used in conjunction with methods the invention. Kits according to this aspect of the invention may comprise one or more containers, which may contain one or more components selected from the group consisting of one or more nucleic acid molecules (e.g., one or more nucleic acid molecules comprising one or more viral sequences and /or one or more recombination sites) and/or viral vectors of the invention, one or more primers, the molecules and/or compounds of the invention, one or more polymerases, one or more reverse transcriptases, one or more recombination proteins (or other enzymes for carrying out the methods of the invention), one or more ligases, one or more buffers, one or more detergents, one or more restriction endonucleases, one or more nucleotides, one or more terminating agents (e.g., ddNTPs), one or more transfection reagents, pyrophosphatase, and the like.

A wide variety of nucleic acid molecules and/or viral vectors of the invention can be used with the invention. Further, due to the modularity of the invention, these nucleic acid molecules can be combined in wide range of ways. Examples of nucleic acid molecules that can be supplied in kits of the invention include those that contain promoters, signal peptides, enhancers, repressors, selection markers, transcription signals, translation signals, primer hybridization sites (e.g., for sequencing or PCR), recombination sites, restriction sites and polylinkers, sites that suppress the termination of translation in the presence of a suppressor tRNA, suppressor tRNA coding sequences, sequences that encode domains and/or regions (e.g., 6 His tag) for the preparation of fusion proteins, origins of replication, telomeres, centromeres, and the like. Similarly, libraries can be supplied in kits of the invention. These libraries may be in the form of replicable nucleic acid molecules or they may comprise nucleic acid molecules that are not associated with an origin of replication. As one skilled in the art would recognize, the nucleic acid molecules of libraries, as well as other nucleic acid molecules that are not associated with an origin of replication, either could be inserted into other nucleic acid molecules that have an origin of replication or would be an expendable kit components.

Further, in some embodiments, libraries supplied in kits of the invention may comprise two components: (1) the nucleic acid molecules of these libraries and (2) 5′ and/or 3′ recombination sites. In some embodiments, when the nucleic acid molecules of a library are supplied with 5′ and/or 3′ recombination sites, it will be possible to insert these molecules into nucleic acid molecules comprising all or a portion of a viral genome, which also may be supplied as a kit component, using recombination reactions. In other embodiments, recombination sites can be attached to the nucleic acid molecules of the libraries before use (e.g., by the use of a ligase, which may also be supplied with the kit). In such cases, nucleic acid molecules that contain recombination sites or primers that can be used to generate recombination sites may be supplied with the kits.

Nucleic acid molecules comprising all or a portion of a viral genome to be supplied in kits of the invention can vary greatly. In some instances, these molecules will contain an origin of replication, at least one selectable marker, and at least one recombination site. For example, molecules supplied in kits of the invention can have four separate recombination sites that allow for insertion of sequence of interest at two different locations of a nucleic acid molecule, for example, as shown in FIG. 2. Other attributes of vectors supplied in kits of the invention are described elsewhere herein.

In some embodiments, the kits of the invention may comprise a plurality of containers, each container comprising one or more nucleic acid segments comprising viral sequences and/or one or more recombination sites and/or topoisomerase recognition sites. Segments may be provided with recombination sites such that a series of segments (e.g., two, three, four, five six, seven, eight, nine, ten, etc.) may be combined in order to construct a viral vector or other nuclei acid molecule of the present invention. Segments may be combined in reactions involving two or more segments (e.g., three, four, five, six, seven, eight, nine, ten, etc.). Each individual segment may be, independently of any other segment, from about 100 bp to about 35 kb in length, or from about 100 bp to about 20 kb in length, or from about 100 bp to about 10 kb in length, or from about 100 bp to about 5 kb in length, or from about 100 bp to about 2.5 kb in length, or from about 100 bp to about 1 kb in length, or from about 100 bp to about 500 bp in length. The present invention also contemplates methods for assembling and using such segments, nucleic acid molecules assembled by such methods, and compositions comprising such nucleic acid molecules.

Segments may be prepared so as to contain viral transcription units. For example, when an adenoviral vector is to be prepared, one segment may comprise, in addition to one or more recombination sites and/or one or more topoisomerase recognition sites, sequences corresponding to the E1 region, the E2 region, the E3 region, and/or the E4 region. Other segments may comprise sequences corresponding to one or more late transcription units and/or viral inverted terminal repeats. Segments comprising nucleic acid sequences of interest may be prepared so as to construct a viral vector or other nucleic acid molecule in which one or more viral nucleic acid sequences, present in a wild-type virus, are not present in the viral vector. Segments comprising a nucleic acid sequence of interest may be prepared and inserted into a viral vector in place of one or more segments comprising viral sequences. In some embodiments, sequences that are present in a wild-type virus but not present in the viral vectors of the invention are those that are not required for replication in cultured cells. For example, a segment comprising a nucleic acid sequence of interest may be used to construct an adenoviral vector wherein the nucleic acid sequence of interest replaces one or more of the E1 region and/or the E3 region. Where necessary (e.g., in the case of the E1 functions) viral functions required to support replication of the viral vector may be supplied in trans (e.g., from the genome of the host cell). Segments may be prepared to construct viral vectors wherein a nucleic acid sequence of interest is place in the viral genome in a position known to be tolerant of nucleic acid insertions, for example, upstream of the E4 region.

A kit of the present invention may comprise a container containing a nucleic acid molecule comprising all or a portion of a viral genome and comprising two recombination sites that do not recombine with each other. The recombination sites may flank a selectable marker that allows selection for or against the presence of the nucleic acid molecule in a host cell or identification of a host cell containing or not containing the nucleic acid. A nucleic acid molecule to be included in a kit may comprise more than two recombination sites, for example, a nucleic acid molecule may comprise multiple pairs of recombination sites (e.g., two, three, four, five, six, seven, eight, nine, ten, etc.) where members of a pair of recombination sites do not recombine or substantially recombine with each other. In some embodiments, members of one pair of recombination sites do not recombine with members of another pair present in the same nucleic acid molecule.

Kits of the invention may comprise containers containing one or more recombination proteins. Suitable recombination proteins have been disclosed above and include, but are not limited to, Cre, Int, IHF, Xis, Flp, Fis, Hin, Gin, Cin, Tn3 resolvase, ΦC31, TndX, XerC, and XerD.

Kits of the invention may also comprise one or more topoisomerase proteins and/or one or more nucleic acids comprising one or more topoisomerase recognition sequence. Suitable topoisomerases include Type IA topoisomerases, Type IB topoisomerases and/or Type II topoisomerases. Suitable topoisomerases include, but are not limited to, poxvirus topoisomerases, including vaccinia virus DNA topoisomerase I, E. coli topoisomerase III, E. coli topoisomerase I, topoisomerase HII, eukaryotic topoisomerase II, archeal reverse gyrase, yeast topoisomerase III, Drosophila topoisomerase III, human topoisomerase III, Streptococcus pneumoniae topoisomerase III, bacterial gyrase, bacterial DNA topoisomerase IV, eukaryotic DNA topoisomerase II, and T-even phage encoded DNA topoisomerases, and the like. Suitable recognition sequences have been described above.

In use, a nucleic acid molecule comprising all or a portion of a viral genome provided in a kit of the invention may be combined with a nucleic acid molecule comprising a sequence of interest using recombinational cloning. The nucleic acid molecule comprising all or a portion of a viral genome may be provided, for example, with two recombination sites that do not recombine with each other. The nucleic acid molecule comprising a sequence of interest may also be provided with two recombination sites, each of which is capable of recombining with one of the two sites present on the a nucleic acid molecule comprising all or a portion of a viral genome. In the presence of the appropriate recombination proteins, the nucleic acid molecule reacts with the nucleic acid molecule comprising all or a portion of a viral genome in order to form a recombinant nucleic acid molecule containing the sequence of interest and all or a portion of a viral genome. When the nucleic acid molecule comprising all or a portion of a viral genome comprises multiple pairs of recombination sites, multiple nucleic acid molecules comprising sequences of interest, which may be the same or different, may be combined with the nucleic acid molecule comprising all or a portion of a viral genome in order to form a nucleic acid molecule comprising all or a portion of a viral genome and also comprises multiple sequence of interest.

Kits of the invention can also be supplied with primers. These primers will generally be designed to anneal to molecules having specific nucleotide sequences. For example, these primers can be designed for use in PCR to amplify a particular nucleic acid molecule. Further, primers supplied with kits of the invention can be sequencing primers designed to hybridize to vector sequences. Thus, such primers will generally be supplied as part of a kit for sequencing nucleic acid molecules that have been inserted into a vector.

One or more buffers (e.g., one, two, three, four, five, eight, ten, fifteen) may be supplied in kits of the invention. These buffers may be supplied at a working concentrations or may be supplied in concentrated form and then diluted to the working concentrations. These buffers will often contain salt, metal ions, co-factors, metal ion chelating agents, etc. for the enhancement of activities of the stabilization of either the buffer itself or molecules in the buffer. Further, these buffers may be supplied in dried or aqueous forms. When buffers are supplied in a dried form, they will generally be dissolved in water prior to use.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

Kits of the invention may comprise one or more pages of written instructions for carrying out the methods of the invention. For example, instructions may comprise methods steps necessary to carry out recombinational cloning of an ORF provided with recombination sites and a vector also comprising recombination sites and optionally further comprising one or more functional sequences.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

The entire disclosures of U.S. appl. Ser. No. 08/486,139, (now abandoned), filed Jun. 7, 1995, U.S. appl. Ser. No. 08/663,002, filed Jun. 7, 1996 (now U.S. Pat. No. 5,888,732), U.S. appl. Ser. No. 09/233,492, filed Jan. 20, 1999, (now U.S. Pat. No. 6,270,969), U.S. appl. Ser. No. 09/233,493, filed Jan. 20, 1999, (now U.S. Pat. No. 6,143,557), U.S. appl. Ser. No. 09/005,476, filed Jan. 12, 1998, (now U.S. Pat. No. 6,171,861), U.S. appl. Ser. No. 09/432,085 filed Nov. 2, 1999, U.S. appl. Ser. No. 09/498,074 filed Feb. 4, 2000, U.S. Appl. No. 60/065,930, filed Oct. 24, 1997, U.S. appl. Ser. No. 09/177,387, filed Oct. 23, 1998, U.S. appl. Ser. No. 09/296,280, filed Apr. 22, 1999, (now U.S. Pat. No. 6,277,608), U.S. appl. Ser. No. 09/296,281, filed Apr. 22, 1999, (now abandoned), U.S. appl. Ser. No. 09/648,790, filed Aug. 28, 2000, U.S. appl. Ser. No. 09/732,914 (published as US 2002 0007051), filed Dec. 11, 2000, U.S. appl. Ser. No. 09/855,797, filed May 16, 2001, U.S. appl. Ser. No. 09/907,719, filed Jul. 19, 2001, U.S. appl. Ser. No. 09/907,900, filed Jul. 19, 2001, U.S. appl. Ser. No. 09/985,448, filed Nov. 2, 2001, U.S. Appl. No. 60/108,324, filed Nov. 13, 1998, U.S. appl. Ser. No. 09/438,358, filed Nov. 12, 1999, U.S. Appl. No. 60/161,403, filed Oct. 25, 1999, U.S. appl. Ser. No. 09/695,065, filed Oct. 25, 2000, U.S. appl. Ser. No. 09/984,239, filed Oct. 29, 2001, U.S. Appl. No. 60/122,389, filed Mar. 2, 1999, U.S. Appl. Ser. No. 60/126,049, filed Mar. 23, 1999, U.S. Appl. No. 60/136,744, filed May 28, 1999, U.S. appl. Ser. No. 09/517,466, filed Mar. 2, 2000, U.S. Appl. No. 60/122,392, filed Mar. 2, 1999, U.S. appl. Ser. No. 09/518,188, filed Mar. 2, 2000, U.S. Appl. No. 60/169,983, filed Dec. 10, 1999, U.S. Appl. No. 60/188,000, filed Mar. 9, 2000, U.S. appl. Ser. No. 09/732,914, filed Dec. 11, 2001, U.S. Appl. No. 60/284,528, filed Apr. 19, 2001, U.S. Appl. No. 60/291,973, filed May 21, 2001, U.S. Appl. No. 60/318,902, filed Sep. 14, 2001, U.S. Appl. No. 60/333,124, filed Nov. 27, 2001, and U.S. appl. Ser. No. 10/005,876, filed Dec. 7, 2001, are herein incorporated by reference.

### EXAMPLES

The present invention provides an extremely versatile method for the modular construction of nucleic acids and production of polypeptides. Both insert nucleic acid segments and the vector can contain sequences selected so as to confer desired characteristics on the product molecules. In some embodiments, in addition to the insert, one or more of the portions of the nucleic acid comprising all or a portion of a viral genome adjacent to the insert, can contain one or more selected sequences. The selected sequences might encode ribozymes, epitope tags, structural domains, selectable markers, internal ribosome entry sequences, promoters, enhancers, recombination sites and the like.

In some embodiments, more than one sequence of interest may be incorporated in a nucleic acid molecule comprising all or a portion of a viral genome. The incorporated sequences of interest may be adjacent to one another or may be separated by a portion of the nucleic acid molecule comprising all or a portion of a viral genome. When separated, the portion of the nucleic acid molecule separating the sequences of interest may comprise one or more selectable markers flanked by a reactive pair of recombination sites in addition to containing the recombination sites used to insert the nucleic acid segments. The portion of the nucleic acid molecule separating the sequences of interest may also comprise viral sequences and/or other sequences conferring a desired characteristic on the nucleic acid molecule and/or sequences of interest.

A sequence of interest may be a sequence of any type. For example, the sequence may encode one or more polypeptides and/or may contain one or more un-translated regions. Sequences of interest may be transcribed and translated into polypeptides or may be transcribed and not translated into polypeptides, for example, anti-sense molecules, ribozymes, and RNAi. Sequences of interest may or may not comprise a stop codon. Sequences comprising a stop codon may or may not comprise additional sequences 3′ to the stop codon that may be in frame with sequences 5′ to the stop codon. In some embodiments, stop codons may be suppressed in order to produce a fusion polypeptide.

Throughout this disclosure, the term gene of interest (GOI) may be used for the sake of convenience. This should not be construed as limiting the present invention to nucleic acid sequences comprising genes. Any nucleic acid sequence of interest can be inserted into a vector of the invention using materials and methods described herein.

### EXAMPLE 1

### Preparation of a Viral Vector of the Invention

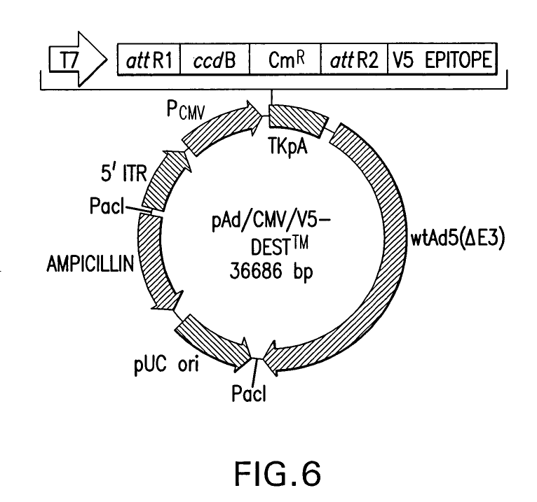

FIG. 6 is a plasmid map of the pAd/CMV/V5-DEST vector, one example of a nucleic acid comprising all or a portion of a viral genome according to the present invention. The nucleotide sequence of the plasmid is provided in Table 6 (SEQ ID NO:). The plasmid contains the first 458 nucleotides of Ad5, including the left ITR and packaging sequence, followed the cytomegalovirus promoter (CMV) and the T7 promoter. The promoters are followed by a sequence containing selectable markers flanked by recombination sites attR1 and attR2. Any other suitable pair of recombination sites might be employed as long as they are selected so as not to recombine with each other. After the attR2 site, the V5 epitope coding sequence is followed by stop codons in all three reading frames and the herpes virus thymidine kinase polyadenylation signal. This is followed by the nucleotides from position 3513 to the right end of the adenoviral genome including the right ITR. After the adenoviral sequences, are plasmid sequences including a plasmid origin of replication followed by the ampicillin resistance gene. The plasmid sequences are flanked by Pacd restriction enzyme recognition sites. Thus, after replacement of the replaceable sequence with a sequence of interest flanked by attL1 and attL2 in a recombination reaction, an infectious viral genome can be prepared by digestion of the recombination reaction product with Pacl to remove the plasmid sequences. In this particular embodiment, the viral genome is an adenoviral genome deleted in the E1 and E3 regions. The E1 function must be supplied in trans in order for the virus to replicate, for example, from the host cell as in 293 cells. The gene products of the E3 region are not required for replication.

In order to prepare a viral vector according to the present invention, a particular sequence of interest may be prepared with recombination sites compatible to those in the pAd/CMV/V5-DEST vector. This may be accomplished using standard techniques, for example, by amplifying a sequences of interest with primers comprising the appropriate recombination site sequences. If a PCR product contains the appropriate recombination site sequences, it may be used directly in a recombination reaction. Optionally, a PCR product or other nucleic acid comprising the sequence of interest may be cloned into a GATEWAY™ entry vector. This can be accomplished using any conventional technique, for example, by a) traditional restriction fragment ligation, b) TOPO-mediated cloning of the nucleic acid comprising the sequence of interest into pENTR-dTOPO, or c) GATEWAY™ clonase reaction between PCR-amplified sequence of interest (e.g., gene of interest (GOI)) containing flanking attB sites with pDONR DNA. Any of these three methods will result in the sequence of interest being inserted into an entry vector. Using the terminology of the GATEWAY™ Technology, the resultant vector would be designated pENTR-GOI for an entry vector comprising a gene of interest (GOI). This should not be construed as limiting the sequences of interest to those encoding genes; any sequence of interest may be inserted into a pENTR vector in this fashion. In this example, this results in the sequence of interest being flanked by attL1 and attL2 recombination sites.

In an in vitro GATEWAY™ LR reaction, the pENTR-GOI vector may be combined with pAd-CMV-DEST. The reaction may be incubated for an appropriate period of time, for example, 1 hour at room temperature. This reaction moves the sequence of interest into the adenoviral vector, pAd-CMV-DEST.

The adenoviral vector containing a sequence of interest is used to transform competent bacteria (i.e., DH5α, TOP10, HB101, etc.). All or a portion of the LR reaction mixture is used to transform competent bacteria and the transformed bacteria are plated on LB-ampicillin bacterial plates and incubated overnight at 37° C.

Several bacterial colonies—2-4 is usually sufficient—may be picked and used to inoculate overnight cultures in LB-ampicillin liquid medium and grown overnight at 37° C.

Plasmid DNA is prepared from the cultures using conventional techniques and analyzed for the presence of the sequence of interest, for example, by restriction enzyme digests or PCR.

To prepare a larger quantity of viral vector, 2 to 5 micrograms of destination vector comprising the sequence of interest may be digested with Pacd restriction enzyme to expose the adenoviral ITRs (immediately adjacent to the Pacd sites on the 5′ and 3′ ends of the adenoviral genome). The digested DNA may be purified using any conventional technique, for example, phenol/chloroform extraction followed by ethanol precipitation, or use of a commercially available kit for this purpose.

The digested DNA is used to transfect an appropriate host cell, for example, 293 cells. The day before transfection, 6 well plates with 5×10⁵293 cells per well may be prepared. On the day of transfection, 2 micrograms of DNA is used to transfect the cells in each well. Transfection may be accomplished using standard techniques using, for example, calcium phosphate, lipids, electroporation, etc. Preferred methods of transfection include those utilizing cationic lipids or mixtures of cationic and neutral lipids. Suitable transfection reagents are commercially available, for example, from Invitrogen Corporation, Carlsbad, Calif. One suitable lipid formulation is Lipofectamine™ 2000.

The day after transfection, the transfection media may be removed and replaced with fresh media. The next day, the transfected cells may be trypsinized and transferred. The cells from one well are used to seed a 100 mm dish. The cells are grown in the 100 mm dish for 7-10 days. The media is replaced with fresh media every 2-3 days. At about 9 days post transfection, “plaques” may be observed forming in the monolayer of 293 cells. Plaques will appear as cleared areas when viewed by the naked eye. Under the microscope, plaques will be fringed with rounded, lysing cells. This is referred to as cytopathic effect (CPE). The media should be replaced with fresh media every 2 days until most of the cells are demonstrating CPE.

Harvest the plate by squirting off the cells using the growth media and transfer the cells and media to a 15 ml tube. Freeze/thaw the tube 3 times by alternating −80° C. and 37° C. This releases the viral particles from the cells. Centrifuge the tube to remove the unwanted cellular debris (3000 rpm×10 minutes). Remove the supernatant and transfer it to a fresh tube. This material now contains recombinant adenoviral vector containing the sequence of interest. This can be used directly in experiments to deliver the sequence of interest.

To increase the titer of the viral vector, the viral vector may be amplified, for example, by applying a small amount (typically 100 microliters) of the initial viral vector to a fresh plate of 293 cells (typically 5×10⁶ 293 cells in a 100 mm dish). Infection of the cells occurs within the first couple hours and three days later CPE is observed throughout the plate. Viral vector is harvested as described above.

Viral vector produced in this way (called “crude viral lysates”, or CVLs) is typically high titer (>10⁹ infectious virus/ml) and can be used directly for most applications. To determine the exact titer of the CVL (or of any adenoviral stock), 293 cells are plated at 1×10⁶ cells per well in 6-well plates. The next day, each well is transduced with 1 ml media containing ten-fold serial dilutions of CVL ranging from 10⁻⁵ to 10⁻¹⁰. After overnight incubation, the media is removed and the cell monolayers are overlaid with 2 ml of fresh media containing 0.4% Ultrapure agarose. This semi-solid medium prevents viral vector from diffusing throughout the plate and keeps individual plaques distinct. After 7 to 10 days, distinct plaques will be visible to the naked eye. MTT (3-(4,5-dimethylthiazol-2-yl)-2,5-diphenyl-tetrazolium bromide) can be used to stain the wells to aid in plaque visualization. Plaques are counted, and that number is multiplied by the dilution factor to obtain the titer of infectious viral vector present in the original CVL. If higher titer viral vector is required, the viral vector in the CVLs can be concentrated and purified using a number of different approaches including: cesium chloride density ultracentrifugation, HPLC, or commercially available columns designed for virus purification (e.g. Virapur). These methods typically result in titers of >11 infectious virus/ml.

### EXAMPLE 2

### Use of Suppressor tRNAs to Generate Fusion Polypeptides

Detection of expressed polypeptides is often facilitated by the use of epitope tags (e.g. V5 or myc) or detectable markers (e.g., β-lactamase, β-galactosidase, β-glucuronidase, GFP, etc.). This is especially useful if there is no specific antibody available for the polypeptide of interest. However, addition of epitope tags and/or fusion to a detectable marker may adversely affect polypeptide activity, structure, or its interaction with other molecules. One common approach to this problem is to clone the gene of interest twice: with and without the tag.

The present invention provides materials and methods to express a polypeptide with and without a tag or marker from the same genetic construct. This is accomplished using mammalian suppressor tRNAs that specifically recognize and decode one of the three stop codons (Ochre, Amber, and Opal) and result in the insertion of an amino acid at the position coded for by the stop codon. The suppressor tRNAs may insert any amino acid into the position coded for by the stop codon. In the specific embodiments described below, the amino acid serine was inserted; however, any amino acid desired can be inserted by preparing and expressing the appropriate suppressor tRNA according to the present invention.

Expression plasmids encoding a reporter gene with all three possible stop codons in frame with C-terminal tags were constructed. Following delivery of suppressor tRNAs in trans, the stop codons between the gene and the epitope tag were suppressed, allowing translation of the 3′ sequences.

Plasmids encoding each suppressor tRNA were co-transfected with the corresponding expression plasmid to test the efficiency of suppression. Suppression of TAA and TAG were approximately 50% to 60% efficient, while TGA was only 30%. Changing the nucleotide following the TGA stop codon from an adenine to a cytosine improved suppression to about 70%.

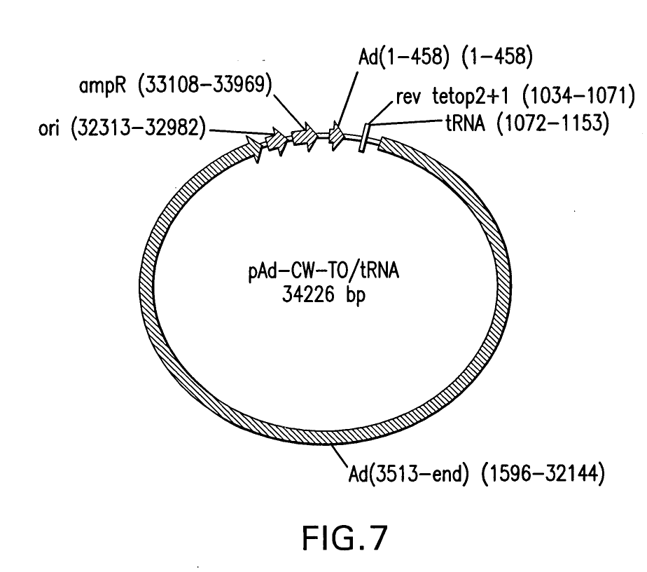

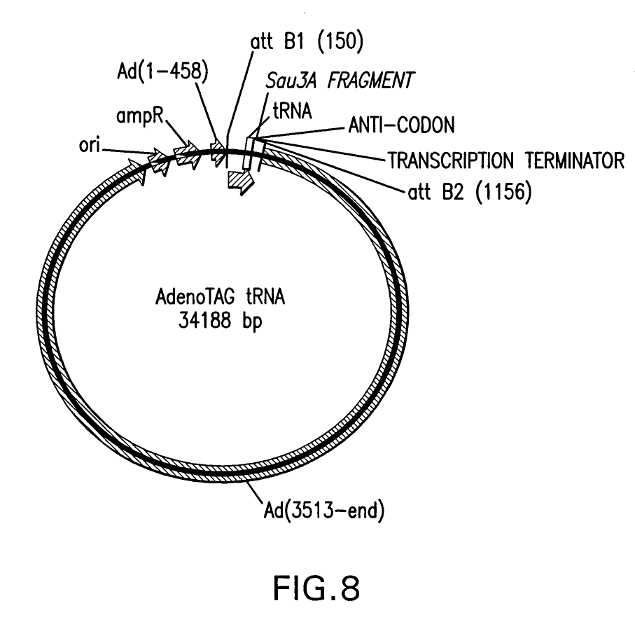

A recombinant adenoviral vector was constructed that expresses a suppressor tRNA. A map of a plasmid containing the adenoviral construct pAd-GW-TO/tRNA in which a suppressor tRNA is under the control of a tetracycline-inducible CMV promoter is shown in FIG. 7. The nucleotide sequence of pAd-GW-TO/tRNA is provided in Table 7 (SEQ ID NO: ). An additional adenoviral construct expressing a suppressor tRNA is pAdenoTAG tRNA shown in FIG. 8. The nucleotide sequence of pAdenoTAG tRNA is provided in Table 8. Table 9 provides the nucleotide sequence of a Sau3A fragment that may be used to construct suppressor tRNA containing nucleic acid molecules of the invention (e.g., pAdenoTag tRNA.) A transcription terminator is located at bases 600 to 606 of the fragment, the sequence corresponding to the suppressor tRNA is located at bases 512 to 593 of the fragment, the anti-codon is located at bases 545 to 547, and the tetracycline operator sequence is located at bases 474 to 511. The suppressor tRNA produced from this sequence suppresses the amber stop codon UAG. Those skilled in the art will appreciated that it is possible to prepare suppressors for opal and ochre stop codons by mutating the bases in the anti-codon to make the anti-codon the reverse complement of the stop codon. i.e., TCA for the opal stop codon and TTA for the ochre stop codon. Other anti-codons may be used, for example, those employing other bases in the wobble position. Constructing a suitable sequence from which to produce a desired suppressor tRNA (e.g., by introducing one or more point mutations in a sequence) is routine in the art.

The plasmid may be digested with Pacd to generate an infectious adenoviral genome. The viral vector expressing the suppressor tRNA may be used in conjunction with any vector comprising a sequence with a stop codon to be suppressed. In some embodiments, a viral vector expressing a suppressor tRNA and a viral vector comprising a sequence of interest may be used to co-infect a cell and produce a fusion polypeptide. A fusion polypeptide may be encoded entirely by the sequence of interest, for example, the sequence may have one open reading frame (ORF) separated from another ORF by a stop codon. Alternatively, one ORF may be present on the sequence of interest and one or more additional ORFs may be present on the viral vector. Co-infection with a suppressor-expressing viral vector an expression vector will result in the expression of a fusion polypeptide; infection without the suppressor-expressing viral vector will produce a native polypeptide. Thus, the suppression technology allows expression of tagged and untagged polypeptides using a single expression vector.

### EXAMPLE 3

### Detailed Materials and Method for Construction of Adenoviral Vectors and Kits

Kits of the invention may comprise one or more sets of instructions for carrying out the methods of the invention. For example, the instructions may related to the propagation of cells used in the methods of the invention and/or to conducting individual reactions that are part of the methods. In a one embodiments, kits of the invention may comprise instructions for growth and maintenance of cell used in methods of the invention (e.g., the 293A cell line manual, catalog no. R705-07 version B, Invitrogen Corporation, Carlsbad, Calif.) and a manual for the preparation of the viral vectors of the invention (e.g., the ViraPower™ Adenoviral Expression System manual, catalog no. K4930-00, version A, Invitrogen Corporation, Carlsbad, Calif.).

In one embodiment, a kit of the invention may comprise the necessary reagents and instructions to prepare a viral vector according to the invention. Such a kit may comprise one or more components selected from the group consisting of: the ViraPower™ Adenoviral GATEWAY™ Expression Kit, ViraPower™ Adenoviral Promoterless GATEWAY™ Expression Kit, pAd/CMV/V5-DEST™ GATEWAY™ Vector Pack, or pAd/PL-DEST™ GATEWAY™ Vector Pack all available from Invitrogen Corporation, Carlsbad, Calif.

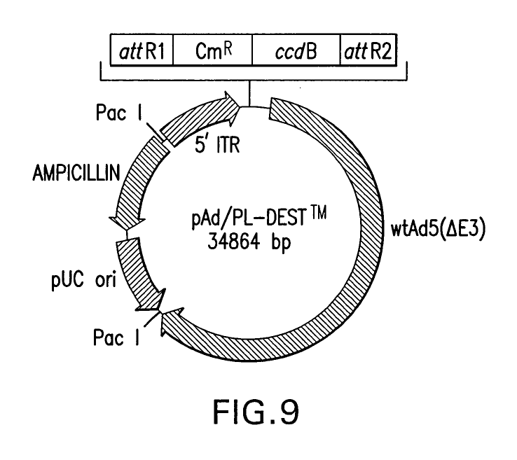

A plasmid map of pAd/PL-DEST™ is provided in FIG. 9 and the sequence of the plasmid is provided in Table 10.

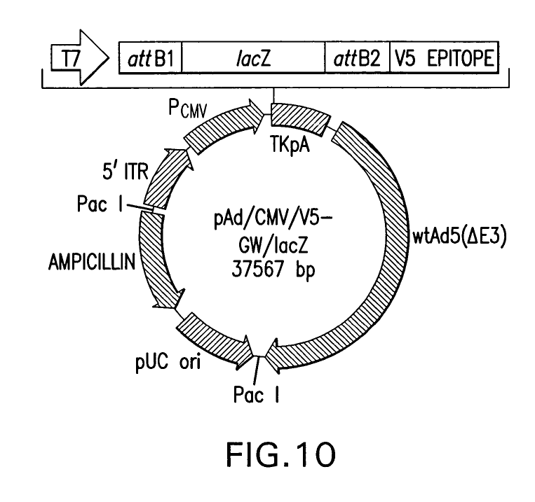

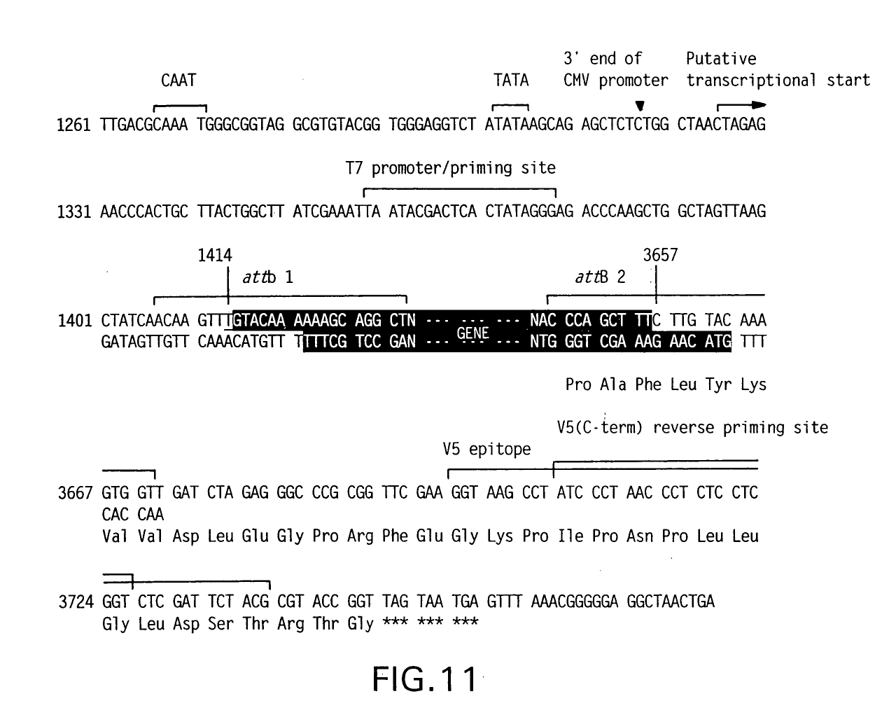

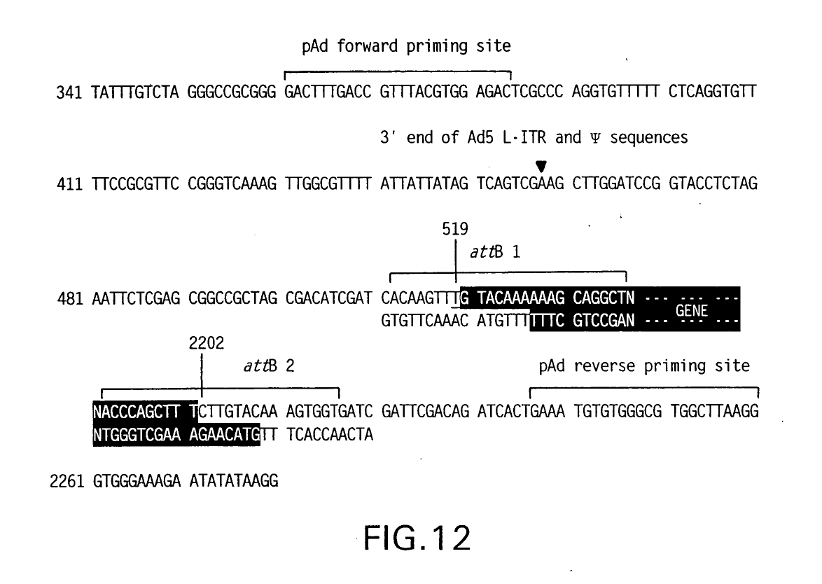

A kit may also comprise one or more control reagents. For example, a kit may comprise an adenoviral vector comprising a detectable marker that may be used as a control for transfection of cells and infection of cells. One suitable control reagent is pAd/CMV/V5-GW/lacZ control. A map of the pAd/CMV/V5-GW/lacZ plasmid is provide as FIG. 10 and the nucleotide sequence of the plasmid is provided as Table 11.

Kits of the invention may comprise one or more additional products (e.g., accessory products). Such products include, but are not limited to, reagents and materials for purifying nucleic acids (e.g., plasmid purification), host cells for propagating plasmids and/or viruses (e.g., E. coli and 293 cells), transfection reagents (e.g., lipids), reagents for assaying control vector expression (e.g., β-lactamase assay reagents, β-galactosidase assay reagents, antibodies to β-galactosidase), recombination polypeptides, and antibiotics for selection of transformed cells. The contents of one suitable kit include, ViraPower™ Adenoviral GATEWAY™ Expression Kit, ViraPower™ Adenoviral Promoterless GATEWAY™ Expression Kit, 293A Cell Line, GATEWAY™ LR Clonase™ Enzyme Mix, Library Efficiency® DB3.1™ Competent Cells, One Shot® TOP10 Chemically Competent E. coli, S.N.A.P.™ MidiPrep Kit, Lipofectamine™ 2000, β-gal Antiserum, and Ampicillin all available from Invitrogen Corporation, Carlsbad, Calif.

A polypeptide encoded by a sequence of interest may be expressed as a fusion polypeptide with a detectable epitope. For example, a polypeptide expressed from pAd/CMV/V5-DEST™ (FIG. 6), can be detected with an antibody to the V5 epitope. Antibodies to the detectable epitope may be labeled, for example, horseradish peroxidase (HRP) or alkaline phosphatase (AP) may be conjugated to the antibody to allow one-step detection using chemiluminescent or colorimetric detection methods. A fluorescent label, (e.g., FITC) may be conjugated to the antibody to allow one-step detection in immunofluorescence experiments. Thus, kits of the invention may comprise one or more antibodies to one or more detectable epitopes. Antibodies to detectable epitopes may be labeled. Suitable antibodies include, but are not limited to, an anti-V5 antibody, an anti-V5-HRP antibody, an anti-V5-AP antibody, and/or an anti-V5-FITC antibody.

Examples of nucleic acid molecules of the invention include pAd/CMV/V5-DEST™ (36.7 kb) and pAd/PL-DEST™ (34.9 kb), which are destination vectors adapted for use with recombinational cloning (e.g., GATEWAY™ Technology), and are designed to allow high-level, transient expression of recombinant fusion polypeptides in dividing and non-dividing mammalian cells, for example, using ViraPower™ Adenoviral Expression System, catalog nos. K4930-00 and K4940-00 available from Invitrogen Corporation, Carlsbad, Calif.

A choice of vectors permits the construction of an adenovirus expressing a sequence of interest. Each vector provides different features that may be useful under different circumstances. For example, the pAd/CMV/V5-DEST™ vector contains the CMV promoter that provides high-level, constitutive expression of the sequence of interest and the C-terminal V5 epitope for detection of recombinant polypeptide using anti-V5 antibodies. The pAd/PL-DEST™ vector has no promoter allowing expression of a sequence of interest from any desired promoter that may be operably linked to the sequence of interest, optionally, prior to insertion in the viral vectors of the invention. Additionally, the pAd/PL-DEST™ vector has no 3′ sequences allowing addition of a C-terminal epitope tag (if desired) and a polyadenylation signal of choice.

The pAd/CMV/V5-DEST™ vector (36686 bp) contains the following features.

| Feature | Benefit |
|---|---|
| Human adenovirus type 5 | Encodes all elements (except E1 |
| sequences (corresponds to | and E3 polypeptides) required to |
| wild-type 1-458 and | produce replication-incompetent |
| 3513-35935 sequence) | adenovirus (Russell, (2000) J. Gen. |
| Note: The E1 and E3 regions | Virol. 81, 2573-2604.) |
| are deleted. | including: |
| | Left and right ITRs |
| | Encapsidation signal for packaging |
| | E2 and E4 regions |
| | Late genes |
| pAd forward priming | Permits sequencing of the |
| site | insert. |
| CMV promoter | Permits high-level expression of |
| | the gene of interest |
| T7 promoter/priming | Allows in vitro transcription in |
| site | the sense orientation and sequencing |
| | through the insert. |
| attR1 and attR2 sites | Bacteriophage λ-derived DNA |
| | recombination sequences that |
| | permit recombinational cloning |
| | of the gene of interest from a |
| | GATEWAY ™ entry clone. |
| ccdB gene | Permits negative selection of |
| | the plasmid. |
| Chloramphenicol | Allows counterselection of the |
| resistance gene (Cm^{R}) | plasmid. |
| V5 epitope | Allows detection of the recombinant |
| | fusion polypeptide by the Anti-V5 |
| | Antibodies |
| Herpes Simplex Virus | Permits efficient transcription |
| thymidine kinase (TK) | termination and polyadenylation |
| ° polyadenylation signal | of mRNA |
| pAd reverse priming site | Allows sequencing of the insert |
| | in the anti-sense orientation. |
| pUC origin | Permits high-copy replication and |
| | maintenance in E. coli. |
| bla promoter | Allows expression of the ampicillin |
| | resistance gene. |
| Ampicillin resistance gene | Allows selection of the plasmid |
| (β-lactamase) | in E. coli. |
| Pac I restriction sites | Permits exposure of the left and |
| (positions 34610 and 36684) | right ITRs required for viral |
| | replication and packaging. |

The pAd/PL-DEST™ vector (34864 bp) contains the following features.

| Feature | Benefit |
|---|---|
| Human adenovirus type 5 | Encodes all elements (except E1 |
| sequences (corresponds | and E3 proteins) required to |
| to wild-type 1-458 | produce replication-incompetent |
| and 3513-35935 | adenovirus (Russell, 2000) |
| sequence) | including: |
| Note: The E1 and E3 | Left and right ITRs |
| regions are deleted. | Encapsidation signal for packaging |
| | E2 and E4 regions |
| | Late genes |
| pAd forward priming site | Permits sequencing of the insert. |
| attR1 and attR2 sites | Bacteriophage λ-derived DNA |
| | recombination sequences that |
| | permit recombinational cloning of |
| | the DNA sequence of interest from |
| | a GATEWAY ™ entry clone (Landy, |
| | 1989, Annu. Rev. Biochem. 58, |
| | 913-949.). |
| Chloramphenicol | Allows counterselection of the |
| resistance gene (Cm^{R}) | plasmid. |
| ccdB gene | Permits negative selection of |
| | the plasmid. |
| pAd reverse priming site | Allows sequencing of the insert |
| | in the anti-sense orientation. |
| pUC origin | Permits high-copy replication and |
| | maintenance in E. coli. |
| bla promoter | Allows expression of the ampicillin |
| | resistance gene. |
| Ampicillin resistance | Allows selection of the plasmid |
| gene (β-lactamase) | in E. coli. |
| Pac I restriction sites | Permits exposure of the left and |
| (positions 32788 and 34862) | right ITRs required for viral |
| | replication and packaging. |

The pAd/CMV/V5-DEST™ and pAd/PL-DEST™ vectors contain the following features: human adenovirus type 5 sequences (Ad 1-458), upstream of the attR1 site, containing the “Left” Inverted Terminal Repeat (L-ITR) and the encapsidation signal sequence required for viral packaging; human cytomegalovirus (CMV) immediate early promoter for high-level constitutive expression of the gene of interest in a wide range of mammalian cells (in pAd/CMV/V5-DEST™ only; (Andersson, et al., 1989, J. Biol. Chem. 264, 8222-8229; Boshart, et al., 1985, Cell 41, 521-530; Nelson, et al., 1987, Molec. Cell. Biol. 7, 4125-4129); two recombination sites, attR1 and attR2 for recombinational cloning of the DNA sequence of interest from an entry clone; chloramphenicol resistance gene (Cm^{R}) located between the two attR sites for counterselection; the ccdB gene located between the attR sites for negative selection; C-terminal V5 epitope for detection of the recombinant polypeptide of interest (in pAd/CMV/V5-DEST™ only); (Southern, et al., 1991, J. Gen. Virol. 72, 1551-1557); human adenovirus type 5 sequences (Ad 3513-35935) containing genes and elements (e.g. E2 and E4 regions, late genes, and “Right” ITR) required for proper packaging and production of adenovirus (Hitt, et al., (1999) In The Development of Human Gene Therapy, T. Friedmann, ed. (Cold Spring Harbor, N.Y.: Cold Spring Harbor Laboratory Press), pp. 61-86.; Russell, (2000)); ampicillin resistance gene for selection in E. coli; and the pUC origin for high-copy replication and maintenance of the plasmid in E. coli. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the destination vector containing attP sites flanking the ccdB and spectinomycin resistance genes can be selected on ampicillin/spectinomycin-containing media. It has recently been found that the use of spectinomycin selection instead of chloramphenicol selection results in an increase in the number of colonies obtained on selection plates, indicating that use of the spectinomycin resistance gene may lead to an increased efficiency of cloning from that observed using cassettes containing the chloramphenicol resistance gene.

The plasmid, pAd/CMV/V5-GW/lacZ, is included and may be used as a positive expression control in the mammalian cell line of choice. pAd/CMV/V5-GW/lacZ (FIG. 10) is a 37567 bp vector expressing β-galactosidase, and was generated using the GATEWAY™ LR recombination reaction between an entry clone containing the lacZ gene and pAd/CMV/V5-DEST™. β-galactosidase is expressed as a C-terminal V5 fusion polypeptide with a molecular weight of approximately 120 kDa.

Nucleic acid molecules of the invention may be constructed using any technique known to those skilled in the art, for example recombinational cloning (e.g., using GATEWAY™). GATEWAY™ is a universal cloning technology that takes advantage of the site-specific recombination properties of bacteriophage lambda (Landy, 1989) to provide a rapid and highly efficient way to move a DNA sequence of interest into multiple vector systems. To express a sequence of interest in mammalian cells using the GATEWAY™ Technology the following method may be used. First, a sequence of interest may be cloned into a GATEWAY™ entry vector of choice to create an entry clone. If pAd-DEST™ is used, a promoter of choice and a polyadenylation signal may be operably attached to the sequence of interest. Next, a recombination reaction (e.g., an LR reaction) may be performed to generate an expression clone by transferring the sequence of interest into a GATEWAY™ destination vector (e.g. pAd/CMV/V5-DEST or pAd-DEST™). An expression clone may then be used to generate viral vector using the ViraPower™ Adenoviral Expression System.

For more information about the GATEWAY™ Technology, generating an entry clone, and performing the LR recombination reaction, refer to the GATEWAY™ Technology manual.

Materials and methods of the invention (e.g., The ViraPower™ Adenoviral Expression System) facilitate highly efficient, in vitro or in vivo delivery of a target gene to dividing and non-dividing mammalian cells using a replication-incompetent adenovirus. The System utilizes GATEWAY™-adapted destination vectors to allow highly efficient and rapid creation of adenoviral vectors that circumvent the need for traditional, homologous recombination and the use of recA⁺ bacteria to produce adenovirus. To express a sequence of interest in mammalian cells using the ViraPower™ Adenoviral Expression System the following method may be used. First, an expression clone in pAd/CMV/V5-DEST™ or pAd-DEST™ may be created (e.g., using GATEWAY™ Technology or other suitable methodology). Next, the expression clone may be digested with Pac I to expose the viral inverted terminal repeats (ITRs). The digested expression clone may be introduced into suitable host cells (e.g., 293 or 293A cells) to produce adenovirus. The adenovirus may be amplified by infecting additional cells and allowing the virus to replicate. The virus may be used to transduce a suitable cell line (e.g., a mammalian cell line of choice). The transduced cell line may be assayed for expression of the sequence of interest using any suitable means.

The pAd/CMV/V5-DEST™ and pAd/PL-DEST™ vectors may be linear or may be supercoiled plasmids. Each destination vector may be supplied as 6 μg of plasmid, lyophilized in TE, pH 8.0. To use, resuspend the destination plasmid in 40 μl of sterile water to a final concentration of 150 ng/μl.

It may be desirable to propagate and maintain the pAd/CMV/V5-DEST™ and pAd/PL-DEST™ vectors. One suitable method is to use Library Efficiency® DB3.1™ Competent Cells (Invitrogen Corporation, Carlsbad, Calif.) for transformation. The DB3.1™ E. coli strain is resistant to CcdB effects and can support the propagation of plasmids containing the ccdb gene. To maintain integrity of the vector, select for transformants in media containing 50-100 μg/ml ampicillin and 15-30 μg/ml chloramphenicol. General E. coli cloning strains including TOP10 or DH5α are not recommended for propagation and maintenance as these strains are sensitive to CcdB effects.

To recombine a sequence of interest into pAd/CMV/V5-DEST™ or pAd-DEST™, the sequence of interest should be cloned into an entry clone. Many entry vectors including pENTR/D-TOPO® are available from Invitrogen Corporation, Carlsbad, Calif. to facilitate generation of entry clones.

pAd/CMV/V5-DEST™ is a C-terminal fusion vector; however, this vector may be used to express native polypeptides or C-terminal fusion polypeptides. A sequence of interest encoding a polypeptide of interest must contain an ATG initiation codon in the context of a Kozak consensus sequence for proper initiation of translation in mammalian cells (Kozak, M. (1987). Nucleic Acids Res. 15, 8125-8148. Kozak, M. (1991). J. Cell Biology 115, 887-903. Kozak, M. (1990). Proc. Natl. Acad. Sci. USA 87, 8301-8305.). An example of a Kozak consensus sequence is (G/A)NNATGG (SEQ ID NO:). The ATG initiation codon is underlined. Note that other sequences are possible, but the G or A at position −3 and the G at position +4 are the most critical for function (shown in bold).

If it is desired to include the V5 epitope tag, a sequence of interest in the entry clone should not contain a stop codon. In addition, the sequence encoding the polypeptide should be in frame with the V5 epitope tag after recombination. To express a native polypeptide (e.g., without a tag sequence) from a sequence of interest, the sequence of interest must contain a stop codon in the entry clone. The C-terminal peptide containing the V5 epitope and the attB2 site will add approximately 4.3 kDa to the size of a polypeptide expressed from a sequence of interest.

pAd/PL-DEST™ allows generation of an adenovirus that contains a sequence of interest whose expression is controlled by a promoter of choice. To facilitate proper expression of a sequence of interest from pAd/PL-DEST™, an entry clone containing the following should be generated: 1) a promoter of choice to control expression of the sequence of interest in mammalian cells; 2). the sequence of interest; 3) a stop codon; and 4) a polyadenylation signal sequence of choice for proper transcription termination and polyadenylation of mRNA. To express a polypeptide from a sequence of interest, the ORF of the polypeptide should contain an ATG initiation codon in the context of a Kozak consensus sequence for proper initiation of translation in mammalian cells (Kozak, 1987; Kozak, 1991; Kozak, 1990). If desired, an N-terminal and/or C-terminal fusion tag sequence may be included.

In some embodiments, an entry clone contains attL sites flanking the sequence of interest. Sequences of interest in an entry clone are transferred to the destination vector backbone by mixing the DNAs with the GATEWAY™ LR Clonase™ Enzyme Mix, Invitrogen Corporation, Carlsbad, Calif. The resulting LR recombination reaction is then transformed into E. coli (e.g. TOP10 or DH5α™-T1^{R}) and the expression clone selected using ampicillin. Recombination between the attR sites on the destination vector and the attL sites on the entry clone replaces the chloramphenicol (Cm^{R}) gene and the ccdB gene with the sequence of interest and results in the formation of attB sites in the expression clone.

The ccdb gene mutates at a very low frequency, resulting in a very low number of false positives. True expression clones will be ampicillin- and blasticidin-resistant and chloramphenicol-sensitive. Transformants containing a plasmid with a mutated ccdB gene will be ampicillin-, blasticidin-, and chloramphenicol-resistant. To check a putative expression clone, test for growth on LB plates containing 30 μg/ml chloramphenicol. A true expression clone should not grow in the presence of chloramphenicol.

The recombination region of the expression clone resulting from pAd/CMV/V5-DEST™×entry clone is shown in FIG. 8. Shaded regions correspond to those DNA sequences transferred from the entry clone into the pAd/CMV/V5-DEST™ vector by recombination. Non-shaded regions are derived from the pAd/CMV/V5-DEST™ vector. Bases 1414 and 3657 of the pAd/CMV/V5-DEST™ sequence are marked. The recombination region of the expression clone resulting from pAd/PL-DEST™×entry clone is shown IN FIG. 9. Shaded regions correspond to those DNA sequences transferred from the entry clone into the pAd/PL-DEST™ vector by recombination. Non-shaded regions are derived from the pAd/PL-DEST™ vector. Bases 519 and 2202 of the pAd/PL-DEST™ sequence are marked.

To confirm that a sequence of interest is in the correct orientation and in frame with a fusion tag (if present), an expression construct may be sequenced. The following primer binding may be used to sequence an expression construct. Refer to the FIGS. 8 and 9 for the location of the primer binding sites. The pAd/CMV/V5-DEST™ vector contains the T7 promoter/priming site 5′-TAATACGACTCACTATAGGG-3′ (SEQ ID NO:) and the V5 (C-term) reverse priming site 5′-ACCGAGGAGAGGGTTAGGGAT-3′ (SEQ ID NO:). The pAd/PL-DEST™ vector contains the pAd forward priming site 5′GACTTTGACCGTTTACGTGGAGAC-3′ (SEQ ID NO:) and the pAd reverse priming site 5′-CCTTAAGCCACGCCCACACATTTC-3′ (SEQ ID NO:).

Once purified plasmid DNA of a pAd/CMV/V5-DEST™ or pAd/PL-DEST™ expression construct has been obtained, the vector may be used in ViraPower™ Adenoviral Expression System (Invitrogen Corporation, Carlsbad, Calif.) by digesting with Pac I. The Pac I-digested vector is used to produce an adenoviral stock, which after amplification, may then be used to transduce a mammalian cell line of choice to express the sequence of interest or a polypeptide encoded by the sequence of interest.

Once a pAd/CMV/V5-DEST and/ or a pAd/PL-DEST™ expression clone has been constructed, purified plasmid DNA may be prepared. Suitable purification methods include the S.N.A.P.™ MidiPrep Kit (Invitrogen Corporation, Carlsbad, Calif.) and CsCl gradient centrifugation. To verify the integrity of an expression construct after plasmid preparation, the plasmid may be analyzed by restriction digests.

Before transfecting an expression clone into 293A cells, the left and right viral ITRs on the vector should be exposed to allow proper viral replication and packaging. Both pAd/CMV/V5-DEST™ and pAd/PL-DEST™ vectors contain Pac I restriction sites. Digestion of the vector with Pac I allows exposure of the left and right viral ITRs and removal of the bacterial sequences (i.e. pUC origin and ampicillin resistance gene). The sequence of interest must not contain any Pac I restriction sites.

Digest at least 5 μg of purified plasmid DNA of pAd/CMV/V5-DEST™ or pAd/PL-DEST expression construct with Pac I using commercially available Pac I enzyme. Follow the manufacturer's instructions. Purify the digested plasmid DNA using phenol/chloroform extraction followed by ethanol precipitation or a DNA purification kit (e.g. S.N.A.P. MiniPrep™ Kit, catalog no. K19001, Invitrogen Corporation, Carlsbad, Calif.). Gel purification is not required.

Resuspend or elute the purified plasmid, as appropriate in sterile water or TE Buffer, pH 8.0 to a final concentration of 0.1-3.0 μg/μl.

To express a gene of interest from pAd/CMV/V5-DEST™ or pAd/PL-DEST™ using Invitrogen's ViraPower™ Adenoviral Expression System, the following reagents are required: 1) a host cell (e.g., 293 or 293A cell lines); and 2) a transfection reagent (e.g., Lipofectamine™ 2000 Reagent, catalog no. 11668019, Invitrogen Corporation, Carlsbad, Calif.). The 293A cell line is a subclone of the 293 cell line and supplies the E1 proteins required for production of replication-competent adenovirus and exhibits a flattened morphology to enhance visualization of plaques.

pAd/CMV/V5-GW/lacZ is included with the each kit for use as a positive control for expression in the ViraPower™ Adenoviral Expression System. In pAd/CMV/V5-GW/lacZ, β-galactosidase is expressed as a C-terminally tagged fusion polypeptide that may be easily detected by western blot or functional assay. To propagate and maintain the plasmid: resuspend the vector in 10 μl of sterile water to prepare a 1 μg/μl stock solution. Use the stock solution to transform a recA, endA E. coli strain like TOP 10, DH5α™-T1^{R}, or equivalent. Use 10 ng of plasmid for transformation. Select transformants on LB agar plates containing 50-100 μg/ml ampicillin. Prepare a glycerol stock of a transformant containing plasmid for long-term storage.

### EXAMPLE 4

### Exemplary Instruction Manual for Kits of the Invention.

Provided for in the methods of the present invention is a kit containing a viral system for high-level, transient expression in dividing and non-dividing mammalian cells. One nonlimiting example of such a kit is the ViraPower™ Adenoviral Expression System, Invitrogen catalog nos. K4930-00 and K4940-00, Version A, Jul. 15, 2002, 25-0543, as described in this example.

The ViraPower™ Adenoviral Expression Kits include the following components. For a detailed description of the contents of each component, see below.

| | Catalog No. | Catalog No. |
|---|---|---|
| Components | K4930-00 | K4940-00 |
| pAd/CMV/V5-DEST ™ | ✓ | |
| GATEWAY ™ Vector | | |
| pAd/PL-DEST ™ GATEWAY ™ | | ✓ |
| Vector | | |
| 293A Cell Line | ✓ | ✓ |

The ViraPower™ Adenoviral Expression Kits are shipped as described below. Upon receipt, store each component as detailed below.

| Item | Shipping | Storage |
|---|---|---|
| pAd-DEST ™ GATEWAY ™ Vector | Blue ice | −20° C. |
| 293A Cell Line | Dry ice | Liquid nitrogen |

Each ViraPower™ Adenoviral Expression Kit includes a destination vector (pAd/CMV/V5-DEST™ or pAd/PL-DEST™) for cloning a DNA sequence of interest and a corresponding expression control vector. For information about the vectors see, for example, the pAd/CMV/V5-DEST™ and pAd/PL-DEST™ GATEWAY™ Vector manual, catalog nos. V493-20 and 494-20, version B, Invitrogen Corporation, Carlsbad, Calif.

Methods of the invention may be practiced using any suitable cell line (e.g., 293A Cell Line, catalog no. R-705-07, Invitrogen Corporation, Carlsbad, Calif.).

A number of reagents that are commercially available may be used in conjunction with the methods of the invention. For example, the following reagents may be obtained from Invitrogen Corporation, Carlsbad, Calif.

| Item | Catalog no. |
|---|---|
| pAd/CMV/V5-DEST ™ GATEWAY ™ Vector | V493-20 |
| pAd/PL-DEST ™ GATEWAY ™ Vector | V494-20 |
| 293A Cell Line | R705-07 |
| Lipofectamine ™ 2000 | 11668-027 |
| | 11668-019 |
| Opti-MEM ® I Reduced Serum Medium | 31985-062 |
| | 31985-062 |
| Phosphate-Buffered Saline (PBS), pH 7.4 | 10010-023 |
| | 10010-031 |
| S.N.A.P ™ MidiPrep Kit | K1910-01 |

The ViraPower™ Adenoviral Expression System allows creation of a replication-incompetent adenovirus that can be used to deliver and express a gene of interest in either dividing or non-dividing mammalian cells. The major components of the ViraPower™ Adenoviral Expression System include: a choice of GATEWAY™-adapted adenoviral vectors that allow highly efficient generation of a recombinant adenovirus containing the gene of interest under the control of the human cytomegalovirus (CMV) immediate-early enhancer/promoter (pAd/CMV/V5-DEST™) or a promoter of choice (pAd/PL-DEST™); a optimized cell line, 293A, which allows production and subsequent, titering of the recombinant adenovirus; and a control expression plasmid containing the lacZ gene which, when packaged into virions and transduced into a mammalian cell line, expresses β-galactosidase. For more information about the adenoviral vectors, the corresponding positive control vector containing the lacZ gene, and GATEWAY™ Technology, refer to the pAd/CMV/V5-DEST™ and pAd/PL-DEST™ GATEWAY™ Vectors manual. This manual is supplied with each ViraPower™ Adenoviral Expression Kit, but may also be obtained by contacting Invitrogen Corporation, Carlsbad, Calif.

Use of the ViraPower™ Adenoviral Expression System to facilitate DNA virus-based expression of the gene of interest provides the following advantages: uses GATEWAY™ Technology to allow highly efficient, rapid cloning of a gene of interest into a full-length adenoviral vector, bypassing the need for a shuttle vector and inefficient homologous recombination in human or bacterial cells; allows generation of high titer adenoviral stocks (i.e., 1×10⁹ pfu/ml in crude preparations and 1×10¹¹ pfu/ml in concentrated preparations); efficiently delivers the gene of interest to actively dividing and non-dividing mammalian cells in culture or in vivo; generates adenoviral constructs with such a high degree of efficiency and accuracy that the system is amenable for use in high-throughput applications or library transfer procedures; and allows production of a replication-incompetent virus that enhances the biosafety of the system and its use as a gene delivery vehicle.

This example provides an overview of the ViraPower™ Adenoviral Expression System and provides instructions and guidelines to: transfect the pAd/CMV/V5-DEST™ or pAd/PL-DEST™ expression construct into the 293A Cell Line to produce an adenoviral stock; amplify the adenoviral stock; titer the adenoviral stock; use the amplified adenoviral stock to transduce any mammalian cell line of choice; and assay for transient expression of any polynucleotide of interest or recombinant polypeptide. This expression may be used to express, for example, a polypeptide, a protein, or an untranslated RNA, e.g., tRNA, all of which are encompassed by the term “gene of interest” as used herein.

For details and instructions to generate an expression construct using pAd/CMV/V5-DEST™ or pAd/PL-DEST™, refer to the pAd/CMV/V5-DEST™ or pAd/PL-DEST™ GATEWAY™ Vector manual. For instructions to culture and maintain the 293A producer cell line, refer to the 293A Cell Line manual. These manuals are supplied with the ViraPower™ Adenoviral Expression Kits, and are also available from Invitrogen Corporation, Carlsbad, Calif.

The ViraPower™ Adenoviral Expression System facilitates highly efficient, in vitro or in vivo delivery of a target gene to dividing and non-dividing mammalian cells using a replication-incompetent adenovirus. Based on the second-generation vectors developed by Bett, A.J., et al., Proc. Natl. Acad. Sci. USA 91:8802-8806 (1994), the ViraPower™ Adenoviral Expression System takes advantage of the GATEWAY™ Technology to simplify and greatly enhance the efficiency of generating high-titer, recombinant adenovirus.

The first major component of the system described in this example is an E1 and E3-deleted, pAd-DEST™-based expression vector into which the gene of interest will be cloned. Expression of the gene of interest is controlled by the human cytomegalovirus (CMV) promoter (in pAd/CMV/V5-DEST™) or the promoter of choice (in pAd/PL-DEST™). The vector also contains the elements required to allow packaging of the expression construct into virions (e.g., 5′ and 3′ ITRs, encapsidation signal, adenoviral late genes). For more information about the pAd-DEST™ expression vectors, refer to the pAd/CMV/V5-DEST™ and pAd/PL-DEST™ GATEWAY™ Vector manual, available from Invitrogen Corporation, Carlsbad, Calif.

The second major component of the system is an optimized 293A Cell Line that will be used to facilitate initial production, amplification, and titering of replication-incompetent adenovirus. The 293A cells contain a stably integrated copy of E1 that supplies the E1 proteins (E1 a and E1 b) in trans that are required to generate adenovirus. For more information about the 293A Cell Line, refer to the 293A Cell Line manual, available from Invitrogen Corporation, Carlsbad, Calif. The pAd-DEST™ vector containing the gene of interest is transfected into 293A cells to produce a replication-incompetent adenovirus. The crude adenoviral stock is used to infect 293A cells to produce an amplified adenoviral stock. Once the adenoviral stock is amplified and titered, this high-titer stock may be used to transduce the recombinant adenovirus into the mammalian cell line of choice for expression of the recombinant polypeptide of interest.

Adenovirus enters target cells by binding to the Coxsackie/Adenovirus Receptor (CAR). After binding to the CAR, the adenovirus is internalized via integrin-mediated endocytosis followed by active transport to the nucleus. Once in the nucleus, the early events are initiated (e.g., transcription and translation of E1 proteins), followed by expression of the adenoviral late genes and viral replication. Expression of the late genes is dependent upon E1. In the ViraPower™ Adenoviral Expression System, E1 is supplied by the 293A producer cells. The viral life cycle spans approximately 3 days. For more information about the adenovirus life cycle and adenovirus biology, refer to the following references as well as published reviews: Bergelson, J. M., et al. Science 275:1320-1323 (1997); Hitt, M.M., et al., “Structure and Genetic Organization of Adenovirus Vectors,” in The Development of Human Gene Therapy, Friedmann, T., ed., Cold Spring Harbor Laboratory Press, Cold Spring Harbor, N.Y. (1999), pp. 61-86.

After adenovirus is transduced into the target cell and is transported to the nucleus, it does not integrate into the host genome. Therefore, expression of the gene of interest is typically detectable within 24 hours after transduction and is transient, only persisting for as long as the viral genome is present. Additional information regarding the use of adenoviral vectors and host cells may be obtained from the following references: Bett, A. J., et al., Proc. Natl. Acad. Sci. USA 91:8802-8806 (1994); Chen, H. H., et al., Hum. Gene Ther. 10:365-373 (1999); Ciccarone, V., et al., Focus 21:54-55 (1999); Dion, L. D., et al., J. Virol. Methods 56:99-107 (1996); Engelhardt, J. F., et al., Nature Genetics 4:27-34 (1993); Fallaux, F. J., et al., Hum. Gene Ther. 9:1909-1917 (1998); Fallaux, F. J., et al., Hum. Gene Ther. 7:215-222 (1996); Fan, X., et al, Hum. Gene Ther. 11:1313-1327 (2000); Graham, F. L., et al., J. Gen. Virol. 36:59-74 (1977); Hitt, M. M., et al., “Structure and Genetic Organization of Adenovirus Vectors,” in The Development of Human Gene Therapy, Friedmann, T., ed., Cold Spring Harbor Laboratory Press, Cold Spring Harbor, N.Y. (1999), pp. 61-86; Kozarsky, K. F., and Wilson, J. M., Curr. Opin. Genet. Dev. 3:499-503 (1993); Krougliak, V., and Graham, F. L., Hum. Gene Ther. 6:1575-1586 (1995); Lochmuller, H., et al., Hum. Gene Ther. 5:1485-1491 (1994); Navarro, V., et al., Gene Ther. 6:1884-1892 (1999); Russell, W. C., J. Gen. Virol. 81:2573-2604 (2000); Wang, I. I., and Huang, I. I., Drug Discovery Today 5:10-16 (2000); Wivel, N. A., “Adenoviral Vectors,” in The Development of Human Gene Therapy, Friedmann, T., ed., Cold Spring Harbor Laboratory Press, Cold Spring Harbor, N.Y. (1999), pp. 87-110; and Zhang, W. W., et al., BioTechniques 18:444-447 (1995).

Viral infection is referred to in some procedures in this example, and viral transduction in other procedures. These terms are defined below.

Infection: Applies to situations where viral replication occurs and infectious viral progeny are generated. Only cell lines that stably express E1 may be infected.

Transduction: Applies to situations where no viral replication occurs and no infectious viral progeny are generated. Mammalian cell lines that do not express E1 are transduced. In this case, an adenovirus is used as a gene delivery vehicle.

The ViraPower™ Adenoviral Expression System is suitable for in vivo gene delivery applications. Many groups have successfully used adenoviral vectors to express a target gene in a multitude of tissues including skeletal muscle, lung, heart, and brain. For more information about target genes that have been successfully expressed in vivo using adenoviral-based vectors, refer to the publications, supra.

The ViraPower™ Adenoviral Expression System includes the following safety features. The entire E1 region is deleted in the pAd/CMV/V5-DEST™ or pAd/PL-DEST™ expression vectors. Expression of the E1 proteins is required for the expression of the other viral genes (e.g., late genes), and thus viral replication only occurs in cells that express E1. Adenovirus produced from the pAd/CMV/V5-DEST™ or pAd/PL-DEST™ expression vectors is replication-incompetent in any mammalian cells that do not express the E1a and E1b proteins. Adenovirus does not integrate into the host genome upon transduction. Because the virus is replication-incompetent, the presence of the viral genome is transient and will eventually be diluted out as cell division occurs. For more information regarding adenoviral transduction and expression, see the publications listed supra.

Despite the presence of the safety features discussed above, the adenovirus produced with this system may still pose some biohazardous risk since it can transduce primary human cells. For this reason, adenoviral stocks generated using this system be handled as Biosafety Level 2 (BL-2) organisms and strictly all published guidelines for BL-2 should be followed. Furthermore, extra caution should be taken when creating adenovirus carrying potential harmful or toxic genes (e.g., activated oncogenes) or when producing large-scale preparations of virus. For more information about the BL-2 guidelines and adenovirus handling, refer to the document, “Biosafety in Microbiological and Biomedical Laboratories,” 4th Edition, published by the Centers for Disease Control (CDC). This document may be downloaded from the CDC Web site.

The genomic copy of E1 in all 293 cell lines contains homologous regions of overlap with the pAd/CMV/V5-DEST™ and pAd/PL-DEST™ vectors. In rare instances, it is possible for homologous recombination to occur between the E1 genomic region in 293 cells and the viral DNA, causing the gene of interest to be replaced with the E1 region, and resulting in generation of a “wild-type,” replication-competent adenovirus (RCA). This event is most likely to occur during large-scale preparation or amplification of virus, and the growth advantages of the RCA allow it to quickly overtake cultures of recombinant adenovirus. To reduce the likelihood of propagating RCA-contaminated adenoviral stocks, caution should be used when handling all viral preparations, which is considered to be BL-2 material. Routine screening for the presence of wild-type RCA contamination after large-scale viral preparations should be performed. Suitable methods to screen for RCA contamination include PCR screening or supernatant rescue assays. If RCA contamination occurs, plaque purification may be performed to re-isolate the recombinant adenovirus of interest. As an alternative, E1-containing producer cell lines such as 911 or PER.C6 which contain no regions of homologous overlap with the adenoviral vectors may be used to help reduce the incidence of RCA generation. For more information regarding RCA, see the publications listed supra, in particular Lochmuller, et al. (1994) and Zhang et al. (1995).

FIG. 13 describes the general steps required to express the gene of interest using the ViraPower™ Adenoviral Expression System. For instructions to generate an adenovirus expression clone using pAd/CMV/V5-DEST™ or pAd/PL-DEST™, refer to the pAd/CMV/V5-DEST™ and pAd/PL-DEST™ GATEWAY™ Vector manual, available from Invitrogen Corporation, Carlsbad, Calif.

First, the adenovirus expression clone containing the gene of interest is generated and digested with Pac I to expose the ITRs according to the methods described herein or by published methods, e.g., the pAd/PL-DEST™ and pAd/CMV/V5-DEST™ manuals, from Invitrogen Corporation, Carlsbad, Calif. Next, the 293A producer cell line is transfected with the adenovirus expression clone. The cells are harvested and lysed to produce a crude viral lysate. The adenovirus may be amplified by infecting 293A producer cells with the crude viral lysate, and the resulting viral stock is titered. The viral stock is used to infect a mammalian cell line of interest, which is then assayed for expression of the gene of interest.

The ViraPower™ Adenoviral Expression System is designed to create an adenovirus to deliver and transiently express a gene of interest in mammalian cells. Although the system has been designed to express any recombinant polypeptide of interest in the simplest, most direct fashion, use of the system is geared towards those users who are familiar with the biology of DNA viruses and adenoviral vectors and possess a working knowledge of viral and tissue culture techniques. For more information about these topics, refer to the following published reviews: Adenovirus biology: see Russell, W. C. J. Gen. Virol. 81:2573-2604 (2000). Adenoviral vectors: see Hitt, M. M., et al., “Structure and Genetic Organization of Adenovirus Vectors,” in The Development of Human Gene Therapy, Friedmann, T., ed., Cold Spring Harbor Laboratory Press, Cold Spring Harbor, N.Y. (1999), pp. 61-86, and Wivel, N. A., “Adenoviral Vectors,” in The Development of Human Gene Therapy, Friedmann, T., ed., Cold Spring Harbor Laboratory Press, Cold Spring Harbor, N.Y. (1999), pp. 87-110. Adenovirus applications: see Wang, I. I., and Huang, I. I., Drug Discovery Today 5:10-16 (2000).

An expression clone may be created containing a DNA sequence of interest in pAd/CMV/V5-DEST™, which expresses the gene of interest under the control of the human CMV promoter, or in pAd/PL-DEST™, which is promoterless, thus allowing the insertion of a cassette containing the gene of interest under the control of any promoter. Refer to the pAd/CMV/V5-DEST™ and pAd/PL-DEST™ GATEWAY™ Vector manual for further instructions. Once an expression clone has been created, any method of preparing purified plasmid DNA that is clean and free from phenol and sodium chloride may be used. Contaminants may kill the cells, and salt may interfere with lipid complexing, decreasing transfection efficiency. Suitable methods of isolating plasmid DNA include, but are not limited to, the S.N.A.P.™ MidiPrep Kit (Catalog No. K1910-01, Invitrogen Corporation, Carlsbad, Calif.) and cesium chloride gradient centrifugation.

Any 293-derived cell line or other cell line that expresses the E1 proteins may be used to produce adenovirus. One such cell lines particularly suited for use in the present invention is the human 293A Cell Line, included with the ViraPower™ Adenoviral Expression kits to facilitate adenovirus production from the E1-deleted pAd-DEST™ vectors. The 293A Cell Line, a subclone of the 293 cell line, supplies in trans the E1 proteins that are required for expression of adenoviral late genes, and thus viral replication. The cell line exhibits a flattened morphology, enabling easier visualization of plaques. For more information about how to culture and maintain 293A cells, refer to the 293A Cell Line manual, available from Invitrogen Corporation, Carlsbad, Calif.

Once an expression clone, for example a pAd-DEST™ expression clone, is created, the expression clone is transfected into a suitable host cell line (e.g., 293A cells) to produce an adenoviral stock. The following section provides protocols and instructions to generate an adenoviral stock, using pAd-DEST™ to illustrate the method of the present invention.

Before transfecting a pAd-DEST™ expression clone into 293A cells, the left and right viral ITRs are exposed to allow proper viral replication and packaging. Each pAd-DEST™ vector contains Pac I restriction sites (refer to the maps of each vector in the pAd/CMV/V5-DEST™ and pAd/PL-DEST™ manual for the location of the Pac I sites). Digestion of the vector with Pac I allows exposure of the left and right viral ITRs and removal of the bacterial sequences (i.e., pUC origin and ampicillin resistance gene). The DNA sequence of interest should not contain any Pac I restriction sites. At least 5 mg of purified plasmid DNA of the pAd-DEST™ expression construct is digested with Pac I (New England Biolabs, Catalog No. R0547S) according to the manufacturer's instructions. The digested plasmid DNA may be purified using phenol/chloroform extraction followed by ethanol precipitation or a DNA purification kit (e.g., Invitrogen's S.N.A.P. MiniPrep™ Kit; catalog No. K1900-01). Gel purification is not required. The purified plasmid is resuspended or eluted, as appropriate, in sterile water or TE Buffer, pH 8.0 to a final concentration of 0.1-3.0 mg/ml.

The following materials are required before beginning: Pac I-digested pAd-DEST™ expression clone containing the DNA sequence of interest (0.1-3.0 mg/ml in sterile water or TE, pH 8.0); pAd/CMV/V5-GW/lacZ positive control vector (supplied with the kit; resuspended in sterile water to a concentration of 1 mg/ml); 293A cells cultured in the appropriate medium (see the 293A Cell Line manual for details); transfection reagent suitable for transfecting 293A cells (e.g., Lipofectamine™ 2000); Opti-MEM® I Reduced Serum Medium (if using Lipofectamine™ 2000; pre-warmed); fetal bovine serum (FBS); sterile 6-well and 10 cm tissue culture plates; and sterile tissue culture supplies, e.g., 15 ml sterile, capped, conical tubes, table-top centrifuge, water bath (set to 37° C.), and cryovials.

The pAd/CMV/V5-GW/lacZ plasmid is included with each ViraPower™ Adenoviral Expression kit as a positive control vector for expression. The positive control vector may be included in the transfection experiment to generate a control adenoviral stock that may be used to help optimize expression conditions in the mammalian cell line of interest. For more information about the positive control vector, refer to the pAd/CMV/V5-DEST™ and pAd/PL-DEST™ GATEWAY™ Vector manual.

Any suitable transfection reagent may be used to introduce the pAd-DEST™ expression construct into 293A cells. Particularly suitable is the cationic lipid-based Lipofectamine™ 2000 Reagent available from Invitrogen. Using Lipofectamine™ 2000 to transfect 293A cells offers several advantages: provides the highest transfection efficiency in 293A cells; DNA-Lipofectamine™ 2000 complexes can be added directly to cells in culture medium in the presence of serum; and removal of complexes or medium change or addition following transfection are not required, although complexes can be removed after 4-6 hours without loss of activity. To facilitate optimal formation of DNA-Lipofectamine™ 2000 complexes, the Opti-MEM® I Reduced Serum Medium available from Invitrogen may be used. For more information about Opti-MEM® I, contact Invitrogen Corporation, Carlsbad, Calif.

Provided below is one method by which adenoviral stocks may be produced in 293A cells using the following optimized transfection conditions below. The amount of adenovirus produced using these recommended conditions is approximately 10 ml of crude viral lysate with a titer ranging from 1×10⁷ to 1×10⁸ plaque-forming units (pfu)/ml. Lipofectamine™ 2000 is one suitable transfection reagent. Other transfection reagents are readily available and may be used according to the appropriate protocols.

| Condition | Amount |
|---|---|
| Tissue culture plate size | 6-well (one well per |
| | adenoviral construct) |
| Number of 293 A cells to transfect | 5 × 10⁵ cells (see |
| | Note below) |
| Amount of Pac I-digested pAd-DEST ™ | 1 μg |
| expression plasmid | |
| Amount of Lipofectamine ™ 2000 | 3 μl |

293A cells are plated 24 hours prior to transfection in complete medium, and should be healthy and 90-95% confluent on the day of transfection.

Provided herein is a method to transfect 293A cells using Lipofectamine™ 2000. One feature of the provided method is that cells may be kept in culture medium during transfection. A positive control and a negative control (no DNA, no Lipofectamine™ 2000) may be included the experiment to aid in evaluation of the results.

The day before transfection, the 293A cells are trypsinized and counted, then plated at 5×10⁵ cells per well in a 6-well plate containing 2 ml of normal growth medium containing serum. On the day of transfection, the culture medium from the 293A cells is removed and replaced with 1.5 ml of normal growth medium containing serum (or Opti-MEM® I Medium containing serum). Antibiotics should not included.

The DNA-Lipofectamine™ 2000 complexes are prepared for each transfection sample as follows: 1 μg of Pac I-digested pAd-DEST™ expression plasmid DNA is diluted in 250 μl of Opti-MEM® I Medium without serum and mixed gently. The Lipofectamine™ 2000 reagent is mixed gently before use, then diluted 3 μl in 250 μl of Opti-MEM® I Medium without serum. The solution is mixed gently and incubated for 5 minutes at room temperature. After the 5 minute incubation, the diluted DNA is combined with the diluted Lipofectamine™ 2000 and mixed gently. The solution is then incubated for 20 minutes at room temperature to allow the DNA-Lipofectamine™ 2000 complexes to form. The solution may appear cloudy, but this will not impede the transfection. The DNA-Lipofectamine™ 2000 complexes is added dropwise to each well and mixed gently by rocking the plate back and forth. The cells are incubated overnight at 37° C. in a CO₂ incubator.

The next day, the medium containing the DNA-Lipofectamine™ 2000 complexes is removed and replaced with complete culture medium (i.e., D-MEM containing 10% FBS, 2 mM L-glutamine, and 1% penicillin/streptomycin). 48 hours post transfection, the cells are trypsinized and transferred to a sterile 10 cm tissue culture plate containing 10 ml of complete culture medium. The recommended guidelines for working with BL-2 organisms should be followed throughout these procedures. The culture medium is replaced with fresh, complete culture medium every 2-3 days until visible regions of cytopathic effect (CPE) are observed (typically 7-10 days post-transfection). The infections proceed until approximately 80% CPE is observed (typically 10-13 days post-transfection). The recombinant adenovirus-containing cells are harvested by squirting cells off the plate with a 10 ml tissue culture pipette. The cells and media are transferred to a sterile, 15 ml, capped tube for lysing as described below.

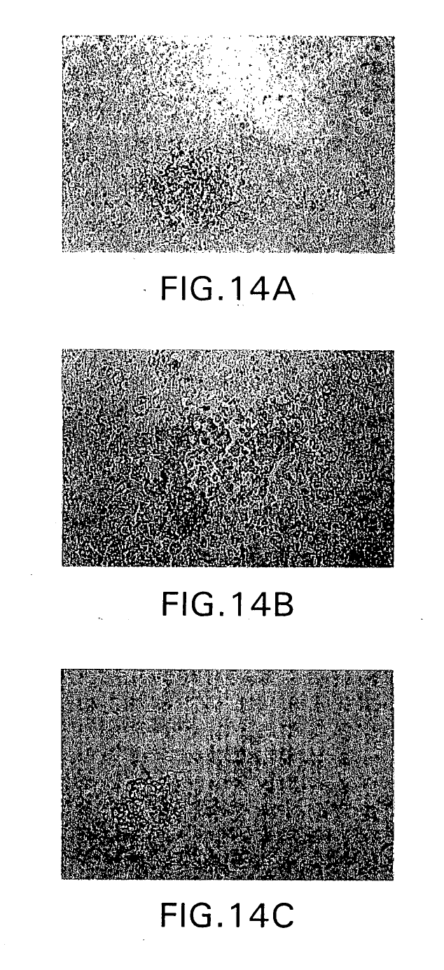

In this example, Pac I-digested pAd/CMV/V5-GW/lacZ plasmid was transfected into 293A cells using the protocol described supra. FIGS. 14A-C show transfected cells as they undergo CPE.

Day 4-6 post-transfection (FIG. 14A): at this early stage, cells producing adenovirus first appear as patches of rounding, dying cells.

Day 6-8 post-transfection (FIG. 14B): as the infection proceeds, cells containing viral particles lyse and infect neighboring cells. A plaque begins to form.

Day 8-10 post-transfection (FIG. 14C): at this late stage, infected neighboring cells lyse, forming a plaque that is clearly visible.

After the adenovirus-containing cells and media are harvested, several freeze/thaw cycles followed by centrifugation may be used to prepare a crude viral lysate. The freeze/thaw cycles cause the cells to lyse and allow release of intracellular viral particles. The tube containing harvested transfected cells and media is placed at −80° C. for 30 minute, then placed in a 37° C. water bath for 15 minutes to thaw. The freezing and thawing steps are repeated twice. The cell lysate is centrifuged in a table-top centrifuge at 3000 rpm for 15 minutes at room temperature to pellet the cell debris. The supernatant containing viral particles, the viral stock, may be transferred to cryovials in 1 ml aliquots and stored at −80° C.

Once a crude viral stock is prepared, it may be amplified by infecting 293A cells as described below. This procedure is recommended to obtain the highest viral titers and optimal results in transduction studies. The titer of the crude viral stock may be determined, and this stock may be used to transduce the mammalian cells of interest to verify the functionality of the adenoviral construct in preliminary expression experiments.

The viral stocks are placed at −80° C. for long-term storage. Because adenovirus is non-enveloped, viral stocks remain relatively stable and some freezing and thawing of the viral stocks is acceptable. Freezing and thawing viral stocks more than 10 times should be avoided as loss of viral titer can occur. When stored properly, viral stocks of an appropriate titer should be suitable for use for up to one year. After long-term storage, re-titering the viral stocks may be performed before use.

Once a crude viral stock is created, this stock may be used to infect 293A cells to generate a higher titer viral stock (i.e., amplify the virus). The titer of the initial viral stock obtained from transfecting 293A cells generally ranges from 1×10⁷ to 1×10⁸ plaque-forming units (pfu)/ml. Amplification allows production of a viral stock with a titer ranging from 1×10⁸ to 1×10⁹ pfu/ml and is generally recommended. Guidelines and protocols are provided in this example to amplify the recombinant adenovirus using 293A cells plated in a 10 cm dish. Larger-scale amplification is possible. Other 293 cell lines or cell lines expressing the E1 proteins are also suitable.

The recommended Federal guidelines for working with BL-2 organisms should be followed for all work with infectious virus. All manipulations should be performed within a certified biosafety cabinet. Media containing virus should be treated with bleach. Used pipettes, pipette tips, and other tissue culture supplies should be treated with bleach or disposed of as biohazardous waste. Gloves, a laboratory coat, and safety glasses or goggles should be worn when handling viral stocks and media containing virus.

Wild-type RCA contamination has not been observed in small-scale (i.e., 3×10⁶ 293A cells plated in a 10 cm dish) adenoviral amplification using the protocol provided below. However, large-scale amplification of virus should be screened for wild-type RCA contamination. Even in large-scale preparations, contamination of adenoviral stocks with wild-type RCA is a rare event.

The following materials are required for amplifying the viral stock: crude adenoviral stock of the pAd-DEST™ construct; sterile 10 cm tissue culture plates; sterile, tissue culture supplies 15 ml sterile, capped, conical tubes; equipment and supplies such as table-top centrifuge, 37° C. water bath, and cryovials.

A typical infection of 293A cells uses the following conditions:

| Condition | Amount |
|---|---|
| Tissue culture plate size | 10 cm (one per adenoviral |
| | construct) |
| Number of 293 A cells to infect | 3 × 10⁶ cells |
| Amount of crude adenoviral stock | 100 μl |
| to use | |

For infection, a 10 cm plate of 293A cells is infected with 100 μl of untitered crude viral stock. Assuming a viral titer of 1×10⁷ to 1×10⁸ pfu/ml, this generally allows harvesting the desired number adenovirus-containing cells 2-3 days after infection. The volume of crude viral stock used to infect cells, may be varied proportionally according to the desired number of cells and/or amount of crude viral stock to as much as 1 ml of crude viral stock. If the titer of the crude viral stock is known, 293A cells are infected at a multiplicity of infection (MOI)=3 to 5.

The procedure below may be used to amplify the adenoviral stock using 293A cells. The day before infection, the 293A cells are trypsinized and counted before plating them at 3×10⁶ cells per 10 cm plate. Cells are plated in 10 ml of normal growth medium containing serum. On the day of infection, the cells are verified to be at 80-90% confluency before proceeding. The desired amount of crude adenoviral stock (e.g., 100 μl) is added to the cells. The plate is swirled gently to mix. The cells are incubated at 37° C. in a CO₂ incubator and the infection is allowed to proceed until 80-90% of the cells have rounded up and are floating or lightly attached to the tissue culture dish (typically 2-3 days post-infection). This CPE indicates that cells are loaded with adenoviral particles. Using less than 100 μl of crude viral stock or a lower titer stock for infection, may require a longer incubation to achieve CPE. The adenovirus-containing cells are harvested by squirting cells off the plate with a 10 ml tissue culture pipette. The cells and media are transferred to a sterile, 15 ml, capped tube which is then placed at −80° C. for 30 minutes. The tube is removed and placed in a 37° C. water bath for 15 minutes to thaw. The freezing and thawing steps are repeated twice. The cell lysate is centrifuged in a table-top centrifuge at 3000 rpm for 15 minutes at room temperature to pellet the cell debris. The supernatant containing viral particles is transferred to cryovials in 1 ml aliquots and may be stored at −80° C.

The amplification procedure is easily scalable to any size tissue culture dish or roller bottle. If it is desirable to scale up the amplification, the number of cells and amount of crude viral stock and medium used is increased in proportion to the difference in surface area of the culture vessel. A screen for the presence of wild-type RCA contamination in the amplified stock may be performed according to suitable screening protocols as described in published literature known to those skilled in the art.

Before proceeding to transduce the mammalian cell line of interest and express the polynucleotide of interest or recombinant polypeptide, determining the titer of the adenoviral stock may be useful. While this procedure is not required for some applications, it is necessary if the number of adenoviral particles introduced to each cell is to be controlled and to generate reproducible expression results. Guidelines and protocols are provided in this example.

To determine the titer of an adenoviral stock, 293A cells are plated in 6-well tissue culture plates. Ten-fold serial dilutions of the adenoviral stock are prepared, then used to infect 293A cells overnight. A plaque assay is performed by first overlaying the infected 293A cells with an agarose/plaquing media solution then allowing 8-12 days for plaques to form. The cells are stained and the number of plaques are counted in each dilution

A number of factors may influence viral titers. Titers generally decrease as the size of the insert increases. The size of the wild-type adenovirus type 5 genome is approximately 35.9 kb. Studies have demonstrated that recombinant adenovirus can efficiently package up to 108% of the wild-type virus size from E1 and E3-deleted vectors. Taking into account the size of the elements required for expression from each pAd-DEST™ vector, the DNA sequence or gene of interest should not exceed the size indicated below for efficient packaging.

| Vector | Insert Size Limit |
|---|---|
| pAd/CMV/V5-DEST ™ | 6.0 kbp |
| Ad/PL-DEST ™ | 7.5 kb |

Other factors include the characteristics of the cell line used for titering and the age of the adenoviral stock. Viral titers may decrease with long-term storage at −80° C. If the adenoviral stock has been stored for 6 months to 1 year, re-titering the adenoviral stock may be performed prior to use in an expression experiment. The number of freeze/thaw cycles and storage of the adenoviral stock may also affect titer. A limited number of freeze/thaw cycles is acceptable, but viral titers may decrease with more than 10 freeze/thaw cycles. Adenoviral stocks may be aliquotted and stored at −80° C.

The 293A cell line supplied with the kit is particularly suitable for use in titering the adenoviral stock, however other cell lines may be used. If another cell line is used, it should: express the E1 proteins, grow as an adherent cell line, be easy to handle, exhibit a doubling time in the range of 18-25 hours, and be non-migratory.

The titer of an adenoviral construct may vary depending on which cell line is chosen. If more than one adenoviral construct is be titered, all of the adenoviral constructs is preferably titered using the same mammalian cell line.

To determine the titer of the adenoviral construct, the following materials are required: the pAd-DEST™ adenoviral stock (stored at −80° C. until use); 293A Cell Line or other appropriate mammalian cell line of choice (see above); complete culture medium for the cell line; 6-well tissue culture plates; 4% agarose (see Recipes; equilibrated to 65° C. before use); plaquing media (normal growth medium containing 2% FBS; equilibrated to 37° C. before use); and 5 mg/ml MTT solution or other appropriate reagent for staining (see Recipes; see below for alternatives).

The vital dye, 3-[4,5-Dimethylthiazol-2-yl]-2,5-diphenyltetrazolium bromide (thiazolyl blue (MTT)) is suitable for use as a staining reagent to help visualize plaques. Other vital stains including Neutral Red (Sigma-Aldrich, St. Louis, Mo., catalog No. N7005) are suitable. To use Neutral Red, a 1% solution (100× stock solution) is prepared in water and stored at +4° C.

The procedure presented herein is a method to determine the titer of the adenoviral stock using the 293A cell line or other appropriate cell line. Other suitable methods are available and known in the art. At least one 6-well plate is required for every adenoviral stock to be titered (six dilutions or one mock well and five dilutions). If an adenoviral stock of the pAd/CMV/V5-GW/lacZ positive expression control has been generated, titering this stock may be done as well. The day before infection (Day 1), the cells are trypsinized and counted for plating at a density such that they will be 80-90% confluent at the time of infection. For example, 293A cells may be used to titer the adenoviral stock and 1×10⁶ cells per well may be plated in each well of a 6-well plate. The cells are incubated at 37° C. overnight.

On the day of infection (Day 2), the adenoviral stock is thawed and diluted 10-fold serially to concentrations ranging from 10⁻⁴ to 10⁻⁹. For each dilution, the adenoviral construct is diluted into complete culture medium to a final volume of 1 ml and mixed by gentle inversion. The culture medium is removed from the cells, and the dilutions are added to one well of cells (total volume=1 ml). The plate is swirled gently to disperse the media, then incubated at 37° C. overnight. The following day (Day 3), the media containing virus is removed and the cells are gently overlaid with 2 ml of Agarose Overlay solution per well.

An agarose overlay solution (enough to overlay one 6-well plate at a time) may be prepared as follows. For one 6-well plate (2 ml overlay per well), 12 ml of pre-warmed (at 37° C.) Plaquing Media and 1.2 ml of pre-warmed (at 65° C.) 4% Agarose is gently mixed while avoiding the formation of bubbles. The overlay is applied to the cells by gently pipetting the overlay down the side of each aspirated well while working quickly to prevent premature solidification. The 6-well plate is placed in a level tissue-culture hood at room temperature for 15 minutes or until the Agarose Overlay solidifies. The plate is returned to a 37° C. humidified CO₂ incubator. 3-4 days following the initial overlay (Day 6-7), the cells are gently overlaid with an additional 1 ml of Agarose Overlay solution (prepared as before) per well. The Agarose Overlay is allowed to solidify before returning the plate to a 37° C. humidified CO₂ incubator. The plates are monitored until plaques are visible (generally 8-12 days post-infection). For each well, the 5 mg/ml MTT solution (1/10 the volume of the Agarose Overlay) is layered gently on top of the solidified agar to stain. For example, if each well contains 3 ml of Agarose Overlay, 300 μl of 5 mg/ml MTT is used. The plates are incubated for 3 hours at 37° C. The plaques are counted to determine the titer of the adenoviral stock.

When titering pAd/CMV/V5-DEST™ or pAd/PL-DEST™ adenoviral stocks using 293A cells, titers ranging from 1×10⁸ to 1×10⁹ pfu/ml are obtained. Adenoviral constructs with titers in this range are generally suitable for use in most applications. If the titer of the adenoviral stock is less than 1×10⁷ pfu/ml, a new adenoviral stock may be produced to increase the titer. See the Troubleshooting section below for more tips and guidelines to optimize the viral yield.

For some applications, viral titers higher than 1×10⁹ pfu/ml may be desired. It is possible to concentrate adenoviral stocks using a variety of methods (e.g., CsCl purification; Engelhardt, J.F., et al., Nature Genetics 4:27-34 (1993), without significantly affecting their transducibility. Use of these methods allows generation of adenoviral stocks with titers as high as 1×10¹¹ pfu/ml.

Once an adenoviral stock with a suitable titer is generated, it may be used to transduce the adenoviral construct into the mammalian cell line of choice and assay for expression of the polynucleotide of interest. Guidelines illustrating one method of transduction are provided below, though it will be appreciated that many such methods are known in the art and may be used in the present invention.

The pAd/CMV/V5-DEST™ or pAd/PL-DEST™ adenoviral construct is replication-incompetent and does not integrate into the host genome. Therefore, once transduced into the mammalian cells of choice, the gene of interest will be expressed only as long as the viral genome is present. The adenovirus terminal protein (TP) covalently binds to the ends of the viral DNA, and helps to stabilize the viral genome in the nucleus. In actively dividing cells, the adenovirus genome is gradually diluted out as cell division occurs, resulting in an overall decrease in transgene expression over time (generally to background levels within 1-2 weeks after transduction). In non-dividing cells (e.g., quiescent CD34+ cells) or animal tissues (e.g., skeletal muscle, neurons), transgene expression is more stable and can persist for as long as 6 months following transduction.

In actively dividing cells (i.e., doubling time of approximately 24 hours), transgene expression is generally detectable within 24 hours of transduction, with maximal expression observed at 48-96 hours (2-4 days) post transduction. Expression levels generally start to decline by 5 days after transduction. In cell lines that exhibit longer doubling times or non-dividing cell lines, high levels of transgene expression typically persist for a longer time. If transducing the adenoviral construct into the mammalian cell line for the first time, a time course of expression may be performed to determine the optimal conditions for expression of the gene of interest.

To obtain optimal expression of the gene of interest, the adenoviral construct may be transduced into the mammalian cell line of choice using a suitable MOI. MOI is defined as the number of virus particles per cell and generally correlates with expression. Typically, expression levels increase linearly as the MOI increases.

A number of factors can influence determination of an optimal MOI including the nature of the mammalian cell line to be used (e.g., non-dividing vs. dividing cell type), its transduction efficiency, the application of interest, and the nature of the gene of interest. If transducing the adenoviral construct into the mammalian cell line of choice for the first time, using a range of MOIs (e.g., 0, 0.5, 1, 2, 5, 10, 20, 50) to determine the MOI required to obtain optimal expression of the DNA or interest or recombinant polypeptide may be performed.

In general, 80-90% of the cells in an actively dividing cell line (e.g., HT1080) express a target gene when transduced at an MOI of ˜1. Other cell types including non-dividing cells may transduce adenoviral constructs less efficiently. If transducing the adenoviral construct into a non-dividing cell type, the MOI may be increased to achieve optimal expression levels for the polynucleotide of interest or recombinant polypeptide.

The pAd/CMV/V5-GW/lacZ control adenoviral construct may be used to determine the optimal MOI for the particular cell line and application. Once the Ad/CMV/V5-GW/lacZ adenovirus is transduced into the mammalian cell line of choice, the gene encoding β-galactosidase will be constitutively expressed and can be easily assayed (refer to the pAd/CMV/V5-DEST™ and pAd/PL-DEST™ GATEWAY™ Vector manual for details, available from Invitrogen Corporation, Carlsbad, Calif.).

Viral supernatants are generated by lysing cells containing virus into spent media harvested from the 293A producer cells. Spent media lacks nutrients and may contain some toxic waste products. If a large volume of viral supernatant is used to transduce the mammalian cell line (e.g., 1 ml of viral supernatant per well in a 6-well plate), growth characteristics or morphology of the target cells may be affected during transduction. These effects are generally alleviated after transduction when the media is replaced with fresh, complete media.

The procedure described herein illustrates one method to transduce the mammalian cell line of choice with the adenoviral construct. Other methods suitable for use with the present invention are readily available for use by one skilled in the art. Mammalian cells of choice are plated in complete media. On the day of transduction (Day 1), the adenoviral stock is thawed, and the appropriate amount of virus is diluted (if necessary) into fresh complete medium. The culture medium is removed from the cells. The medium containing virus is mixed gently by pipetting and add to the cells. The plate is swirled gently to disperse the medium, then incubated at 37° C. overnight. On the following day (Day 2), the medium containing virus is removed and replaced with fresh, complete culture medium. The cells are harvested (if needed) on the desired day (e.g., 2 days post transduction) and assayed for expression of the polynucleotide of interest or recombinant polypeptide.

Any method of choice to detect the polynucleotide of interest or recombinant polypeptide of interest including functional analysis, immunofluorescence, northern blot, or western blot. If the gene of interest is cloned in frame with an epitope tag, the recombinant polypeptide of interest may be detected using an antibody to the epitope tag (see the pAd/CMV/V5-DEST™ and pAd/PL-DEST™ GATEWAY™ Vector manual for details, available from Invitrogen, Carlsbad, Calif.).

Troubleshooting

Below are listed some potential problems and possible solutions that may help troubleshoot the cotransfection and titering experiments.

| Problem | Reason | Solution |
|---|---|---|
| Low viral | Low transfection efficiency: | Repeat the Pac I digestion. Make |
| titer | Incomplete Pac I digestion or | sure that the purified DNA is not |
| | digested DNA contaminated | contaminated with phenol, ethanol, or |
| | with phenol, ethanol, or salts | salts. Use healthy 293A cells; do not |
| | Unhealthy 293A cells; cells | overgrow. Cells should be 90-95% |
| | exhibit low viability 293A | confluent at the time of transfection. |
| | cells plated too sparsely | Optimize such that plasmid DNA (in |
| | Plasmid DNA:transfection | μg): Lipofectamine ™ 2000 (in μl) |
| | reagent ratio incorrect | ratio ranges from 1:2 to 1:3. If using |
| | | another transfection reagent, optimize |
| | | according to the manufacturer's |
| | | recommendations. |
| | Viral supernatant too dilute | Concentrate virus using CsCl |
| | | purification (Engelhardt, J. F., et al., |
| | | Nature Genetics 4: 27-34 (1993) or |
| | | any method of choice. |
| | Viral supernatant frozen and | Do not freeze/thaw viral supernatant |
| | thawed multiple times | more than 10 times. |
| | Gene of interest is large | Viral titers generally decrease as the |
| | | size of the insert increases; inserts |
| | | larger than 6 kb (for pAd/CMV/V5-DEST ™) |
| | | and 7.5 kb (for pAd/PL-DEST ™) are |
| | | not recommended. |
| | Gene of interest is toxic to | Generation of constructs containing |
| | cells | activated oncogenes or potentially |
| | | harmful genes is not recommended. |
| No plaques | Viral stocks stored | Aliquot and store stocks at −80° C. |
| obtained | incorrectly | Do not freeze/thaw more than 10 times. |
| upon titering | Incorrect titering cell line | Use the 293A cell line or any cell line |
| | used | with the characteristics discussed. |
| | Agarose overlay incorrectly | Make sure that the agarose is not too |
| | prepared | hot before addition to the cells; |
| | | hot agarose will kill the cells. |

Transducing Mammalian Cells

Below are listed some potential problems and possible solutions that may help troubleshoot the transduction and expression experiment.

| Problem | Reason | Solution |
|---|---|---|
| Titer | Viral supernatant not diluted | Titer adenovirus using 10-fold serial |
| indeterminable; | sufficiently | dilutions ranging from 10⁻⁴ to 10⁻⁹. |
| cells confluent | | |
| No expression | Viral stocks stored | Aliquot and store stocks at −80° C. |
| | incorrectly | Do not freeze/thaw more than 10 |
| | | times. |
| | Gene of interest contains a | Perform mutagenesis to change or |
| | Pac I site | remove the Pac I site. |
| Poor expression | Poor transduction efficiency: | |
| | Mammalian cells not healthy | Make sure that the cells are healthy |
| | Non-dividing cell type used | before transduction. |
| | | Transduce the adenoviral construct |
| | | into cells using a higher MOI. |
| | MOI too low | Transduce the adenoviral construct |
| | | into cells using a higher MOI. |
| | Low viral titer | Amplify the adenoviral stock using |
| | | the procedure. |
| | Adenoviral stock | Screen for RCA contamination |
| | contaminated with RCA | (Dion, L. D., et al., J. Virol. Methods |
| | | 56: 99-107 (1996)). Prepare a new |
| | | adenoviral stock or plaque purify to |
| | | isolate recombinant adenovirus. |
| | Cells harvested too soon | Do not harvest cells until at least |
| | after transduction | 24-48 hours after transduction. |
| | Cells harvested too long | For actively dividing cells, assay for |
| | after transduction | maximal levels of recombinant |
| | | polypeptide expression within 5 days |
| | | of transduction. |
| | Gene of interest is toxic to | Generation of constructs containing |
| | cells | activated oncogenes or potentially |
| | | harmful genes is not recommended. |
| Persistent | Too much crude viral stock | Reduce the amount crude viral stock |
| toxicity in | used | used for transduction or dilute the |
| target cells | | crude viral stock. |
| | | Amplify the adenoviral stock. |
| | | Concentrate the crude viral stock. |
| | Wild-type RCA | Screen for RCA contamination |
| | contamination | (Dion, L. D., et al., J. Virol. Methods |
| | | 56: 99-107 (1996); Zhang, W. W., et |
| | | al., BioTechniques 18: 444-447 |
| | | (1995). Plaque purify to isolate |
| | | recombinant adenovirus or prepare a |
| | | new adenoviral stock. |

### Recipes

4% Agarose

This procedure may be used to prepare a 4% Agarose solution.

Materials Needed: Ultra Pure Agarose (Invitrogen, Catalog No. 15510-027) Deionized, sterile water.

Protocol: Prepare a 4% stock solution in deionized, sterile water.

Autoclave at 121° C. for 20 minutes to sterilize. Equilibrate to 65° C. in a water bath and use immediately or store at room temperature indefinitely. If the agarose solution is stored at room temperature, melting the agarose is required before use. To melt, microwave the agarose to melt, then equilibrate to 65° C. in a water bath before use.

5 mg/ml MTT

This procedure may be used to prepare a 5 mg/ml MTT solution.

Materials Needed: 3-[4,5-Dimethylthiazol-2-yl]-2,5-diphenyl-tetrazolium bromide; Thiazolyl blue (MTT; Sigma-Aldrich, St. Louis, Mo., Catalog No. M2128). Phosphate-Buffered Saline (PBS; Invitrogen, Catalog No. 10010-023).

Protocol: Prepare a 5 mg/ml stock solution in PBS. Filter-sterilize and dispense 5 ml aliquots into sterile, conical tubes. Store at +4° C. for up to 6 months.

### Example 5

The present invention provides materials and methods for the stable expression of heterologous polypeptides in cells (e.g., insect cells). pIB/V5-His-DEST and pIB/V5-His-GW/lacZ are nucleic acid molecules of the invention that are commercially available from Invitrogen Corporation, Carlsbad, Calif. Information concerning the construction and use of these vectors may be found in Catalog no. 12550-018 Version A, Jul. 15, 2002, 25-0607, available from Invitrogen Corporation, Carlsbad, Calif.

Nucleic acid molecules of the invention may be used to express a polypeptide of interest as part of a fusion polypeptide. Numerous suitable fusion partners are known to those in the art. For example a polypeptide of interest may be expressed as a fusion polypeptide containing the V5 epitope. Antibodies to detect the V5 epitope, a 14 amino acid epitope derived from the P and V proteins of the paramyxovirus, SV5 having the sequence GKPIPNPLLGLDST (Southern, J. A., et al., J. Gen. Virol. 72:1551-1557 (1991)) are commercially available from Invitrogen Corporation, Carlsbad, Calif., for example, Anti-V5 Antibody catalog no. R960-25, Anti-V5-HRP Antibody catalog no. R961-25, and catalog no. Anti-V5-AP Antibody R962-25. A polypeptide of interest may be expressed as a fusion polypeptide with a polyhistidine sequence. Antibodies to detect a polyhistidine sequence are commercially available from Invitrogen Corporation, Carlsbad, Calif. For example, Anti-His(C-term) Antibody catalog no. R930-25, Anti-His(C-term)-HRP Antibody catalog no. R931-25, and Anti-His(C-term)-AP Antibody R932-25, all of which detect a C-terminal polyhistidine (6×His) tag and require the free carboxyl group for detection (i.e., detect the sequence HHHHHH-COOH, see Lindner, P., et al., BioTechniques 22:140-149 (1997)).

An open reading frame present on a sequence of interest may be cloned in frame with the C-terminal peptide containing the V5 epitope and the polyhistidine (6×His) and Immobilized Metal Affinity Chromatography (IMAC) may be used to purify the recombinant fusion polypeptide. The ProBond™ Purification System as well as the Ni-NTA Purification System are available from Invitrogen Corporation, Carlsbad, Calif.

| Product | Catalog no. |
|---|---|
| ProBond ™ Purification System | K850-01 |
| ProBond ™ Nickel-chelating Resin | R801-01 |
| | R801-15 |
| ProBond ™ Purification System with | K853-01 |
| Anti-His(C-term)-HRP Antibody | |
| ProBond ™ Purification System with | K854-01 |
| Anti-V5-HRP Antibody | |
| Purification Columns (10 ml | R640-50 |
| polypropylene columns) | |
| Ni-NTA Purification System | K950-01 |
| Ni-NTA Agarose | R901-01 |
| | R901-15 |
| Ni-NTA Purification System with | K953-01 |
| Anti-His(C-term)-HRP Antibody | |
| Ni-NTA Purification System with | K954-01 |
| Anti-V5-HRP Antibody | |

pIB/V5-His-DEST is a 5.0 kb vector derived from pIB/V5-His and adapted for use with GATEWAY™ Technology. It is designed to allow transient or stable expression of a sequence of interest, which may encode a polypeptide, in insect cell lines.

pIBNV5-His-DEST contains the following features:

| Feature | Benefit |
|---|---|
| OpIE2 promoter | Allows constitutive expression of |
| | the gene of interest in lepidopteran |
| | insect cells (Theilmann, D. A., and |
| | Stewart, S., Virology 187: 84-96 |
| | (1992)) |
| attR1 and attR2 sites | Allows recombinational cloning of |
| | the gene of interest from an entry |
| | clone. |
| Chloramphenicol | Allows counterselection of |
| resistance gene (Cm^{R}) | expression clones. |
| ccdB gene | Allows negative selection of |
| | expression clones. |
| V5 epitope | Allows detection of a recombinant |
| | polypeptide with the Anti-V5 |
| | Antibodies (Southern, J. A., et al., |
| | J. Gen. Virol. 72: 1551-1557 |
| | (1991)) |
| C-terminal poly- | Allows purification of recombinant |
| histidine tag | polypeptides on metal-chelating resin |
| | such as ProBond ™ or Ni-NTA. |
| | Allows detection of the recombinant |
| | polypeptide by the Anti-His (C-term) |
| | Antibodies (Lindner, P., et al., |
| | BioTechniques 22: 140-149 (1997)) |
| OpIE2 polyadenylation | Efficient transcription termination |
| sequence | and polyadenylation of mRNA (Theilmann, |
| | D. A., and Stewart, S., Virology 187: |
| | 84-96 (1992)) |
| pUC origin | Allows high-copy number replication |
| | and growth in E. coli. |
| GP64 promoter | Allows constitutive expression of the |
| | blasticidin resistance gene in |
| | lepidopteran insect cells (Blissard, |
| | G. W., et al., Virology 190: 783-793 |
| | (1992); Blissard, G. W., and Rohrmann, |
| | G. F., J. Virology 65: 5820-5827 |
| | (1991)) |
| EM7 promoter | Allows efficient expression of the |
| | blasticidin and ampicillin resistance |
| | genes in E. coli. |
| Blasticidin | Allows generation of stable insect |
| resistance gene (bsd) | cell lines (Kimura, M., et al., |
| | Biochim. Biophys. ACTA 1219: |
| | 653-659 (1994)) |
| Ampicillin resistance | Allows selection of transformants |
| gene (bla) | in E. coli |
| | Note: The native promoter has been |
| | removed. Transcription is assumed to |
| | start from the EM7 promoter. |

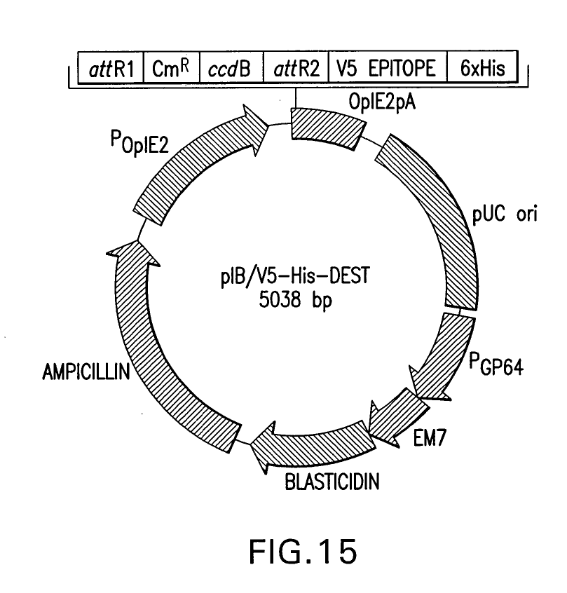

A map of pIB/V5-His-DEST is provided in FIG. 15 and the nucleotide sequence of the vector is provided in Table 12.

GATEWAY™ is a universal cloning technology that takes advantage of the site-specific recombination properties of bacteriophage lambda (Landy, 1989) to provide a rapid and highly efficient way to move a gene of interest into multiple vector systems. To express a sequence of interest using GATEWAY™ Technology: clone the sequence of interest into a GATEWAY™ entry vector to create an entry clone; generate an expression clone by performing an LR recombination reaction between the entry clone and a GATEWAY™ destination vector (e.g. pIB/V5-His-DEST); and introduce the expression clone into insect cells for transient or stable expression.

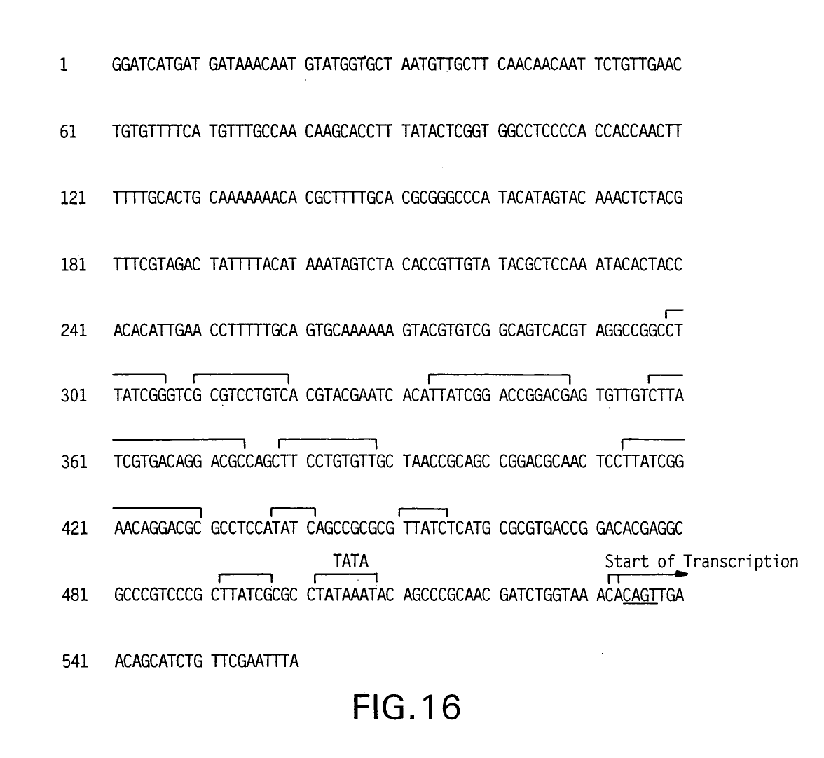

Baculovirus immediate-early promoters utilize the host cell transcription machinery and do not require viral factors for activation. The OpIE2 promoter is from the baculovirus Orgyia pseudotsugata multicapsid nuclear polyhedrosis virus (OpMNPV) and drives constitutive expression of the gene of interest in pIB/V5-His-DEST. The virus' natural host is the Douglas fir tussock moth; however, the promoter allows protein expression in Lymantria dispar (LD652Y), Spodoptera frugiperda cells (Sf9) (Hegedus, D. D., et al., Gene 207:241-249 (1998); Pfeifer, T. A., et al., Gene 188:183-190 (1997)), Sf21 (Invitrogen), Trichoplusia ni (High Five™, Invitrogen Corporation, Carlsbad, Calif.), Drosophila (Kc1, S2) (Hegedus, D. D., et al., Gene 207:241-249 (1998); Pfeifer, T. A., et al., Gene 188:183-190 (1997)) and mosquito cell lines. The OpIE2 promoter has been sequenced and analyzed. The sequence of the promoter is provided in FIG. 16.

Although the OpIE2 promoter provides relatively high levels of constitutive expression, some proteins may not be expressed at levels seen with baculovirus late promoters such as polyhedrin or very late promoters such as p10 (Jarvis, D. L., et al., Protein Expression and Purification 8:191-203 (1996)). Typical expression levels range from 1-2 μg/ml (human IL-6; Invitrogen) to 8-10 μg/ml (human melanotransferrin) (Hegedus, D. D., et al., Protein Expression and Purification 15:296-307 (1999)).

The OpIE2 promoter has been analyzed by deletion analysis using a CAT reporter in both Lymantria dispar (LD652Y) and Spodoptera frugiperda (Sf9) cells. Expression in Sf9 cells was much higher than in LD652Y cells. Deletion analysis revealed that sequence up to −275 base pairs from the start of transcription is necessary for maximal expression (Theilmann, D. A., and Stewart, S., Virology 187:84-96 (1992)). Additional sequence beyond −275 may broaden the host range expression of this plasmid to other insect cell lines In addition, an 18 bp element appears to be required for expression. This 18 bp element is repeated almost completely in three different locations and partially at six other locations. These are marked in FIG. 16. Elimination of the three major 18 bp elements reduces expression to basal levels (Theilmann, D. A., and Stewart, S., Virology 187:84-96 (1992)). Primer extension experiments revealed that transcription initiates equally from either the C or the A indicated. These two transcriptional start sites are adjacent to a CAGT sequence motif that has been shown to be conserved in a number of early genes (Blissard, G. W., and Rohrmann, G. F., Virology 170:537-555 (1989)).

The GP64 promoter regulates expression of the baculovirus major envelope glycoprotein gene (GP64) of the budded virion. Studies have shown that while the GP64 promoter is stimulated by the transcriptional transactivator IE-1, low levels of activity still occur without transactivation (Blissard, G. W., et al., Virology 190:783-793 (1992); Blissard, G. W., and Rohrrmann, G. F., J. Virology 65:5820-5827 (1991)). Furthermore, deletion analysis has identified the specific region required for transcriptional initiation in the absence of IE-1 (Blissard, G. W., et al., Virology 190:783-793 (1992); Blissard, G. W., and Rohrmann, G. F., J. Virology 65:5820-5827 (1991)).

pIB/V5-His-DEST contains a 100 bp region of the Autographa californica nuclear polyhedrosis virus (AcMNPV) GP64 promoter which is sufficient for activation of the blasticidin resistance gene (bsd) in the absence of any baculovirus proteins. Using standard blasticidin concentrations (10-80 μg/ml), stable transfectants will only be selected if the bsd gene is expressed at suitable levels. Without wishing to be bound by theory, because of the minimal activity of the GP64 promoter, it is likely that only stable transfectants containing pIB/V5-His-DEST integrated into the most transcriptionally active genomic loci will be selected. This allows generation of stable cell lines which will express higher levels of the protein of interest compared to cell lines expressing the bsd gene product from the OpIE1 promoter, as in the parent pIB/V5-His vector.

Cell cultures of either Sf9 (catalog no. B82501, Invitrogen Corporation, Carlsbad, Calif.), Sf21 (catalog no. B82101, Invitrogen Corporation, Carlsbad, Calif.), or High Five™ cells (catalog no. B85502, Invitrogen Corporation, Carlsbad, Calif.) may be used in connection with the present invention and may be grown and stored using conventional techniques well known in the art (e.g., Baculoviral Expression Systems and Insect Cell Lines manual, Feb. 27, 2002, Invitrogen Corporation, Carlsbad, Calif.).

The pIB/V5-His-DEST vector is supplied as a supercoiled plasmid. Linearization of this vector is not required to obtain optimal results for any downstream application. The vector may be resuspended at a concentration of 50-150 ng/μl in sterile water, pH 8.0. To propagate and maintain pIB/V5-His-DEST, Library Efficiency® DB3.1™ Competent Cells (Invitrogen Corporation, Carlsbad, Calif. Catalog no. 11782-018) may be used. The DB3.1™ E. Coli strain is resistant to CcdB effects and can support the propagation of plasmids containing the ccdB gene. To maintain integrity of the vector, select for transformants in media containing 50-100 μg/ml ampicillin and 15 μg/ml chloramphenicol. The use of general E. coli cloning strains including TOP10 or DH5α is not recommended for propagation and maintenance as these strains are sensitive to CcdB effects. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the pcDNA destination vector containing attP sites flanking the ccdB and spectinomycin resistance genes can be selected on ampicillin/spectinomycin-containing media. It has recently been found that the use of spectinomycin selection instead of chloramphenicol selection results in an increase in the number of colonies obtained on selection plates, indicating that use of the spectinomycin resistance gene may lead to an increased efficiency of cloning from that observed using cassettes containing the chloramphenicol resistance gene.

To recombine a sequence of interest into pIB/V5-His-DEST, an entry clone containing the sequence of interest may be prepared. A commercially available kit (e.g., the pENTR Directional TOPO® Cloning Kit, Invitrogen Corporation, Carlsbad, Calif. Catalog no. K2400-20, version B) can be used. Other suitable entry vectors are available from Invitrogen Corporation, Carlsbad, Calif. Detailed information on constructing an entry clone may be obtained from the manual provided with the specific entry vector. For detailed information on performing the LR recombination reaction, refer to the GATEWAY™ Technology manual, Invitrogen Corporation, Carlsbad, Calif.

A sequence of interest may contain a Kozak consensus sequence with an ATG initiation codon for proper initiation of translation (Kozak, M., Nucleic Acids Res. 15:8125-8148 (1987); Kozak, M., J. Cell Biology 115:887-903 (1991); Kozak, M., Proc. Natl. Acad. Sci. USA 87:8301-8305 (1990)). An example of a Kozak consensus sequence is provided below. Other sequences are possible, but the G or A at position −3 and the G at position +4 are the most critical for function (shown in bold). The ATG initiation codon is shown underlined.

(G/A)NNATGG

To include the V5 epitope and/or 6×His tag encoded by the vector, the sequence of interest may not contain a stop codon. A coding sequence should also be designed to be in frame with the C-terminal epitope tag after recombination. To express a polypeptide with a native C-terminal (i.e., without the V5 epitope and/or 6×His tag), the sequence of interest should contain a stop codon in the entry clone.

Each entry clone contains attL sites flanking the sequence of interest. Sequences of interest in an entry clone may be transferred to the destination vector backbone by mixing the DNAs with the GATEWAY™ LR Clonase™ enzyme mix. The resulting LR recombination reaction may then be transformed into E. coli and the expression clone may be selected. In an embodiment, recombination between the attR sites on the destination vector and the attL sites on the entry clone replaces the ccdB gene and the chloramphenicol (Cm^{R}) gene with the sequence of interest and results in the formation of attB sites in the expression clone.

The LR Clonase™ reaction; subsequent transformation of a suitable E. coli, and selection for an expression clone may be performed using standard techniques such as those provide in the GATEWAY™ Technology manual.

The ccdB gene mutates at a very low frequency, resulting in a very low number of false positives. True expression clones will be ampicillin-resistant and chloramphenicol-sensitive. Transformants containing a plasmid with a mutated ccdB gene will be both ampicillin- and chloramphenicol-resistant. A putative expression clone can be tested by growth on LB plates containing 30 μg/ml chloramphenicol. A true expression clone will not grow in the presence of chloramphenicol.

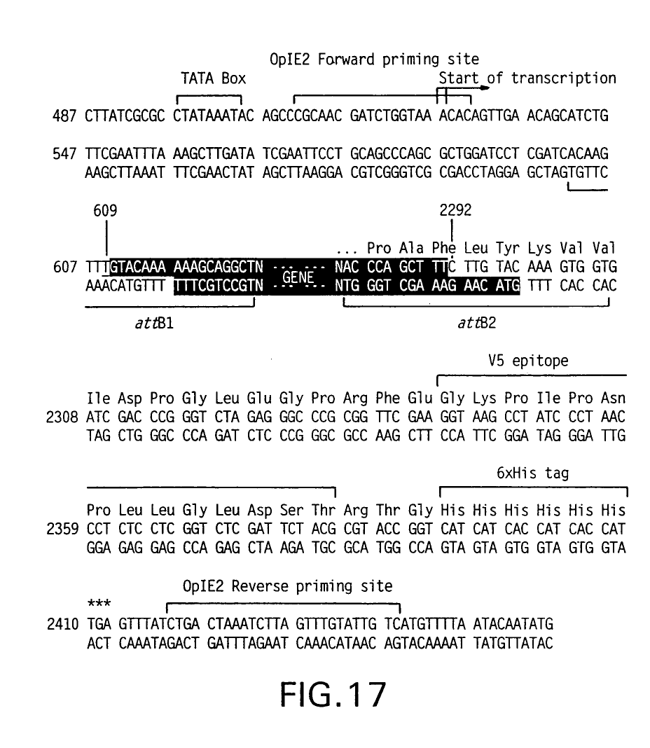

The recombination region of the expression clone resulting from pIB/V5-His-DEST×entry clone is shown in FIG. 17. Shaded regions correspond to those DNA sequences transferred from the entry clone into pIB/V5-His-DEST by recombination. Non-shaded regions are derived from the pIB/V5-His-DEST vector. The underlined nucleotides flanking the shaded region correspond to bases 609 and 2292, respectively, of the pIB/V5-His-DEST vector sequence.

To confirm that a coding sequence on the sequence of interest is in frame with the C-terminal V5 epitope and polyhistidine tag, the expression construct may be sequenced, for example, using the OpIE2 Forward and Reverse primer sequences. Refer to FIG. 17 for the sequence and location of the primer binding sites.

Plasmid DNA for transfection into insect cells must be very clean and free from phenol and sodium chloride. Contaminants will kill the cells, and salt will interfere with lipid complexing, decreasing transfection efficiency. The expression construct plasmid may be prepared using standard techniques, for example, column chromatography(e.g., the S.N.A.P.™ MiniPrep Kit Catalog no. K1900-01, Invitrogen Corporation, Carlsbad, Calif.). Typical yields of plasmid using this technique are 10-15 μg of plasmid DNA from 10-15 ml of bacterial culture. Plasmid can be used directly for transfection of insect cells.

One technique suitable to introduce the nucleic acid molecules of the invention into host cells is lipid-mediated transfection (e.g., using Cellfectin® Reagent, catalog no. 10362010, Invitrogen Corporation, Carlsbad, Calif.). Other lipids may be substituted, although transfection conditions may have to be optimized. Expected Transfection Efficiency using Cellfectin® Reagent: 40-60% for Sf9 or Sf21 cells and 40-60% for High Five™ cells. Other transfection methods (e.g., calcium phosphate and electroporation (Mann and King, 1989)) may also be used with High Five™ cells.

Controls may be included in the transfection reaction, for example, IB/V5-His-GW/lacZ vector as a positive control for transfection and expression and lipid only as a negative control DNA only to check for DNA contamination.

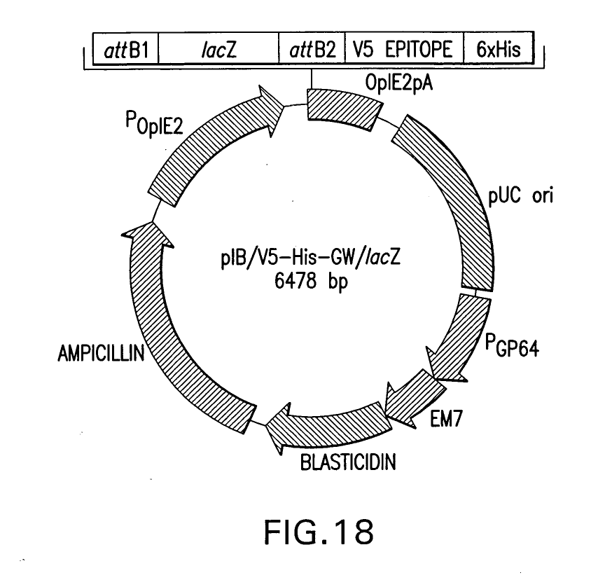

pIB/V5-His-GW/lacZ is provided as a positive control vector for transfection and expression (see FIG. 18 for a map). The vector allows expression of a C-terminally tagged β-galactosidase fusion polypeptide that may be detected by Western blot or functional assay. pIB/V5-His-GW/lacZ is a 6478 bp control vector containing the gene for β-galactosidase. pIB/V5-His-GW/lacZ was constructed using the GATEWAY™ LR recombination reaction between an entry clone containing the lacZ gene and pIB/V5-His-DEST. β-galactosidase is expressed as a fusion to the C-terminal tag. The molecular weight of the fusion polypeptide is approximately 120 kDa.

To propagate and maintain the plasmid: resuspend the vector in 10 μl sterile water to prepare a 1 μg/μl stock solution and use the stock solution to transform a recA, endA E. coli strain like TOP10, DH5a, JM109, or equivalent. Select transformants on LB agar plates containing 50-100 μg/ml ampicillin. Optionally, a glycerol stock of a transformant containing plasmid may be prepared for long-term storage.

For each transfection, log-phase cells with greater than 95% viability may be used. A time course for expression of the sequence of interest may be performed. For example, expression of a polypeptide encoded by the sequence of interest may be assayed for at 2, 3, and 4 days post transfection. One or more 60 mm plate may be used for each time point. For Sf9, Sf21, or High Five™ cells, 1×10⁶ cells may be seeded in appropriate serum-free medium in a 60 mm dish. Rock gently from side to side for 2 to 3 minutes to evenly distribute the cells. Cells may be 50 to 60% confluent.

Incubate the cells for at least 15 minutes without rocking to allow the cells to fully attach to the bottom of the dish to form a monolayer of cells.

Verify that the cells have attached by inspecting them under an inverted microscope.

Nucleic acid molecules of the invention may be introduced into host cells using standard techniques. A protocol for use of Cellfectin® Reagent is provided below. Other conditions for transfection may be empirically determined by one skilled in the art using routine experimentation. Preferably, a plasmid is not linearized prior to introduction into a host cell. Linearizing a plasmid appears to decrease protein expression. The reason for this is not known.

A suitable transfection may employ: 1-10 μg of purified pIB/V5-His-DEST expression construct (˜1 μg/μl in TE buffer); either log-phase Sf9 or Sf21 cells (1.6-2.5×10⁶ cells/ml, >95% viability) or log-phase High Five™ cells (1.8-2.3×10⁶ cells/ml, >95% viability), growing in serum-free medium (e.g., Grace's Medium without supplements; serum-free medium 60 mm tissue-culture dishes; 1.5 ml sterile microcentrifuge tubes; rocking platform only (NOT orbital); 27° C. incubator; inverted microscope; paper towels and air-tight bags or containers; and 5 mM EDTA, pH 8.

Transfection may comprise mixing plasmid DNA and Cellfectin® in an appropriate medium and incubating with freshly seeded insect cells. The amount of cells, liposomes, and plasmid DNA described herein has been optimized for 60 mm culture plates. Other transfection conditions may be used with other size plates or flasks. Optimizing conditions for other volumes of transfection may be accomplished by one skilled in the art using routine experimentation. Serum-free medium (e.g., Sf-900 II SFM (catalog no. 1090207) to transfect Sf9 or Sf21 cells and Express Five® SFM (catalog no. 10486017) to transfect High Five™ cells, available from Invitrogen Corporation, Carlsbad, Calif.) can be used. Grace's Medium without supplements may also be used. The proteins in the FBS and supplements will interfere with the liposomes, causing the transfection efficiency to decrease.

To prepare each transfection mixture, a 1.5 ml microcentrifuge tube may be used. The following reagents may be added: 1 ml of Grace's Medium OR appropriate serum-free medium; 1-10 μl nucleic acid molecule of the invention (e.g., pIB/V5-His plasmid or construct) at a concentration of ˜1 μg/μl in TE, pH 8; 20 μl Cellfectin® Reagent (mixed well before use and always added last). The transfection mixture may be mixed gently for 10 seconds and incubated at room temperature for 15 minutes.

The medium covering the cells to be transfected should be removed without disrupting the monolayer. If the medium contained serum, wash the cells by carefully adding 2 ml of fresh Grace's Medium without supplements or FBS to remove trace amounts of serum that will decrease the efficiency of liposome transfection and remove the wash.

The entire transfection mix described above may be added dropwise into the 60 mm dish. The drops may be evenly distributed over the monolayer. This method reduces the chances of disturbing the monolayer. Repeat for all transfections.

The dishes may be incubated at room temperature for 4 hours on a side-to-side, rocking platform. A suitable speed for the platform is ˜2 side to side motions per minute. Instead of a platform rocker, the dishes may be manually rocked periodically.

Following the 4-hour incubation period, 1-2 ml of complete TNM-FH medium (Sf9 or Sf21 cells) or the appropriate serum-free medium (Sf9, Sf21, or High Five™ cells) may be added to each 60 mm dish. The dishes may be placed in a sealed plastic bag with moist paper towels to prevent evaporation and incubated at 27° C. It is not necessary to remove the transfection solution as Cellfectin® Reagent is not toxic to the cells. If a different lipid is used and loss of viability is observed, then remove the transfection solution after 4 hours, rinse twice with medium, and replace with 1-2 ml of fresh medium.

The cells may be harvested, for example, at 2, 3, and 4 days post transfection and assayed for expression of the sequence of interest. Additional fresh medium need not be added to the cells if the cells are sealed in an airtight plastic bag with moist paper towels.

Expression of a sequence of interest from the expression clone can be performed in transiently transfected cells or stable cell lines. A sample protocol to detect by Western blot a polypeptide encoded by a sequence of interest expressed as a fusion polypeptide is provided below.

The cells from one 60 mm plate may be used for each expression experiment. A suitable cell lysis buffer may be used. One suitable buffer is 50 mM Tris, pH 7.8, 150 mM NaCl, 1% Nonidet P-40.

The medium may be removed from the cells. If the polypeptide expressed from the sequence of interest is predicted to be secreted, save and assay both the medium and the cell pellet. Cell lysis buffer, 100 μl, may be added to the plate and the cells may be sloughed or scraped into a microcentrifuge tube. The cells may be vortexed to ensure they are completely lysed. The lysed cells may be centrifuged at maximum speed in a microfuge for 1-2 minutes to pellet nuclei and cell membranes. The supernatant may be transferred to a new tube. If a membrane protein is expressed from the sequence of interest, it may be located in the pellet. The pellet and the lysate may be assayed. The protein concentration in the lysate may be determined, for example, by the Bradford, Lowry, or BCA assays (Pierce).

Samples may be mixed with SDS-PAGE sample buffer as follows: 30 μl lysate with 10 μl 4×SDS-PAGE sample buffer; the pellet may be resuspended in 100 μl 1×SDS-PAGE sample buffer; 30 μl medium may be mixed with 10 μl 4×SDS-PAGE sample buffer. Because of the volume of medium, it is difficult to normalize the amount loaded on an SDS-PAGE gel. Optionally, the medium may be concentrated to facilitate normalization. Samples may be boiled for 5 minutes, centrifuged briefly, and approximately 3 to 30 μg protein loaded per lane of an SDS-PAGE gel. The same volume of sample may be added for both the pellet sample and the lysate sample. The amount to load may be determined by one skilled in the art using routine experimentation. Samples may be separated by electrophoresis, blotted, and probed with a suitable antibody using standard techniques.

A polypeptide expressed from a sequence of interest as a fusion polypeptide may be detected by Western blot analysis, for example, with the Anti-V5 antibodies or the Anti-His(C-term) antibodies available from Invitrogen Corporation, Carlsbad, Calif. or an antibody that specifically recognizes the polypeptide. In addition, the Positope™ Control Protein (Invitrogen Corporation, Carlsbad, Calif., Catalog no. R900-50) is available for use as a positive control for detection of fusion proteins containing a V5 epitope or a 6×His tag.

If the pIB/V5-His-GW/lacZ plasmid is used as a positive control vector, β-galactosidase expression may be assayed by Western blot analysis or activity assay (Miller, J. H., Experiments in Molecular Genetics, Cold Spring Harbor Laboratory, Cold Spring Harbor, N.Y. (1972)). Commercially available antibodies (e.g., Invitrogen Corporation, Carlsbad, Calif., β-Gal Antiserum, Catalog no. R901-25), or assay kits (e.g., Invitrogen Corporation, Carlsbad, Calif. β-Gal Assay Kit, Catalog no. K1455-01 and β-Gal Staining Kit Catalog no. K1465-01) may be used for detection of β-galactosidase expression.

The C-terminal peptide containing the V5 epitope and the polyhistidine tag will add approximately 5 kDa in molecular weight to a polypeptide expressed from a sequence of interest.

Selecting Stable Cell Lines

Stable expression cell lines can be created for long-term storage and large-scale production of the desired polypeptide. Note that stable cell lines are created by multiple copy integration of the vector. Amplification as in the case with calcium phosphate transfection and hygromycin resistance in Drosophila is generally not observed.

Blasticidin may be used to select for stably transformed cells. Gloves, mask, goggles, and protective clothing (e.g. a laboratory coat) should be worn when handling blasticidin. Weighing blasticidin and preparing solutions should be done in a hood. Blasticidin may be inactivated for disposal by adding sodium bicarbonate. Blasticidin is soluble in water and acetic acid. Water is generally used to prepare stock solutions of 5 to 10 mg/ml. Blasticidin may be dissolved in sterile water and filter-sterilized. Blasticidin is unstable in solutions with a pH greater than 8.0. The pH of a solution of blasticidin may be 7.0. Blasticidin solutions may be divided into aliquots in small volumes and frozen at −20° C. for long-term storage or stored at +4° C. for short term storage. Aqueous stock solutions are stable for 1-2 weeks at +4° C. and 6-8 weeks at −20° C. Stock solutions should not be subjected to multiple freeze/thaw cycles (do not store in a frost-free freezer). Solutions should be discarded after 1-2 weeks storage at +4° C.

Cytopathic effects should be visible within 3-5 days depending on the concentration of blasticidin in the medium. Sensitive cells will enlarge and become filled with vesicles. The outer membrane will show signs of blebbing, and cells will eventually detach from the plate. Blasticidin-resistant cells should continue to divide at regular intervals to form distinct colonies. There should not be any distinct morphological changes between blasticidin-resistant cells compared to cells not under selection with blasticidin.

In general, concentrations of blasticidin around 10 μg/ml will kill Sf9 or Sf21 cells (in complete TNM-FH medium) and concentrations around 20 μg/ml will kill High Five™ cells (in Express Five® SFM) within one week, although a few cells may remain that exclude trypan blue. To obtain faster and more thorough killing, 50-80 μg/ml blasticidin may be used. Once blasticidin-resistant clones have been obtained, cells may be maintained in lower concentrations of blasticidin (e.g., 10-20 μg/ml). An appropriate concentration of blasticidin for any specific cell type may be determined by one skilled in the art by performing a kill curve.

A suitable protocol for establishing a kill curve is provided. Assays may be conducted in 24-well tissue culture plates. Suitable medium (e.g., TNM-FH medium or the serum-free medium of choice) may be prepared and supplemented with concentrations ranging from 0 to 100 μg/ml blasticidin. Generally, concentrations that effectively kill lepidopteran insect cells within a week are in the 50 to 80 μg/ml range. While 10-20 μg/ml blasticidin will kill cells within a week, higher concentrations will result in faster and more thorough killing. In addition, using higher concentrations of blasticidin may result in enrichment of clones containing multiple integrations of a sequence of interest. Test varying concentrations of blasticidin on a cell line of interest to determine the concentration that kills the cells within a week (kill curve). The concentration of drug that kills the cells of interest within a week should be used.

To isolate a stable cell line, a mock transfection and a positive control (e.g., pIB/V5-His-GW/lacZ) may be used. Cells may be transfected as described above. Forty-eight hours post transfection, the transfection solution may be removed and fresh medium containing no blasticidin may be added. The cells may be split 1:5 (20% confluent) and allowed to attach overnight before adding selective medium. The medium may be removed and replaced with medium containing blasticidin at the appropriate concentration. The cells may be incubated at 27° C. The selective medium may be replaced every 3 to 4 days until foci are observed. Cloning cylinders or limiting dilution may be used to isolate clonal cell lines. Optionally, resistant cells may be allowed to continue grow out to confluence for a polyclonal cell line (2 to 3 weeks).

A polyclonal cell line may be isolated by allowing the resistant cells grow to confluence and splitting the cells 1:5. The polyclonal cell line may be tested for expression. Medium without blasticidin should be used when splitting cells and cells should be allowed to attach before adding selective medium.

Resistant cells may be expanded into flasks to prepare frozen stocks. Medium containing blasticidin should be used when maintaining stable lepidopteran cell lines. The concentration of blasticidin may be lowered to 10 pg/ml for maintenance.

Isolation of Clonal Cell Lines Using Cloning Cylinders

Multiple foci may be isolated for expression testing. As in mammalian cell culture, the location of integration may affect expression of a sequence of interest. Selections may be performed in small plates or wells. Cells should not be allowed to dry out during the selection.

The closed plate may be examined under a microscope and the location of one or more colony marked on the top of the plate. The markings may then be transferred to the bottom of the plate. Orientation marks may be included. Each colony may contain 50 to 200 cells. Sf9 cells tend to spread more than High Five™ cells. The culture dish may be moved to a sterile cabinet and the lid removed. A thin layer of sterile silicon grease may be applied to the bottom of a cloning cylinder (Scienceware, Catalog no. 378747-00 or Belco, Catalog no. 2090-00608), using a sterile cotton-tipped wooden applicator. The layer should be thick enough to retard the flow of liquid from the cylinder, without obscuring the opening on the inside. Cloning cylinders and silicon grease can be sterilized together by placing a small amount of grease in a glass petri dish and placing the cloning cylinders upright in the grease. After autoclaving, the grease will have spread out in a thin layer to coat the bottom of the cylinders.

The culture medium may be removed and the cylinder placed firmly and directly over the marked area. A microscope may be used to direct placement of the cylinder. 20 to 100 μl of medium (no blasticidin) may be used to dislodge the cells. The cells and medium may be removed and transferred to a microtiter plate and the cells may be allowed to attach. The medium may be removed and replaced with selective medium for culturing. The cell line may be expanded and tested for expression of the sequence of interest.

Isolation of Clonal Cell Lines Using a Dilution Method

Clonal cell lines may be established using a dilution method. The objective of this method is to dilute the cells so that under selective pressure only one stable viable cell per well is achieved. The higher transfection efficiency, the more the cells should be diluted. The protocol below works well with cells transfected at 5-10% efficiency.

Forty-eight hours after transfection, cells may be diluted to 1×10⁴ cells/ml in medium without blasticidin. Other dilutions of the culture may also be used as transfection efficiency will determine how many transformed cells there will be per well. 100 μl of the cell solution may be added to 32 wells of a 96-well microtiter plate (8 rows by 4 columns). The remaining cells may be diluted 1:1 with medium without blasticidin and add 100 μl of this solution added to the next group of 32 wells (8×4). The remaining cells may be diluted 1:1 with medium without blasticidin and 100 μl of this solution added to the last group of 32 wells. Although the cells can be diluted to low numbers, cell density is critical for viability. If the density drops below a certain level, the cells will not grow.

The cells may be allowed to attach overnight, then the medium removed and replaced with medium containing blasticidin. Removing and replacing medium may be tedious. Optionally, it is possible to dilute the cells directly into selective medium if they are handled gently.

The plate may be wrapped and incubated at 27° C. for 1 week. It is not necessary to change the medium or place in a humid environment. The plate may be checked after a week and the wells that have only one colony may be marked. The plate may be incubated until the colony fills most of the well. The cells may be harvested and transferred to a 24-well plate with 0.5 ml of fresh medium containing blasticidin. The clone may be expanded to 12- and 6-well plates, and finally to a T-25 flask.

Each cell line may be assayed for yield of the desired polypeptide and the one with the highest yield may be scaled-up and used for purification of recombinant polypeptide. For secreted polypeptides, the cell pellet as well as the medium may be assayed. The yield of polypeptide in the cells may be compared to the yield of polypeptide in the medium.

Master stocks and working stocks of stable cell lines may be prepared prior to scale-up and purification.

Purification

A polypeptide expressed from a sequence of interest may be purified using standard techniques. Stable cell lines prepared as described above may be expanded into larger flasks, spinners, shake flasks, or bioreactors to obtain the desired yield of polypeptide. If a polypeptide expressed from a sequence of interest is secreted, cells may be cultured in serum-free medium to simplify purification.

A 6His tagged fusion polypeptide may be purified using the ProBond™ Purification System, the Ni-NTA Purification System, or a similar product. Both purification systems contain a metal-chelating resin specifically designed to purify 6×His-tagged polypeptides.

Cells may be maintained in a medium having a concentration of blasticidin of 10 μg/ml. Cells may be switched from complete TNM-FH medium to serum-free medium during passage.

Adding serum-free medium directly to a metal-chelating resin such as ProBond™ to purify a secreted polypeptide from serum-free medium will strip the nickel ions from the resin. To purify 6×His-tagged recombinant polypeptides from the culture medium, dialysis or ion exchange chromatography may be performed prior to affinity chromatography on metal-chelating resins. Dialysis allows removal of media components that strip Ni⁺² from metal-chelating resins. Ion exchange chromatography allows removal of media components that strip Ni⁺² from metal-chelating resins and concentration of sample for easier manipulation in subsequent purification steps.

Conditions for successful ion exchange chromatography will vary depending on the polypeptide. For more information, refer to Coligan, J. E., et al., Current Protocols in Protein Science, Chanda, V. B., ed., John Wiley and Sons, Inc., New York (1998), Ausubel, F. M., et al., Current Protocols in Molecular Biology, Unit 10 (1994), or Deutscher, M. P., “Guide to Protein Purification,” in Methods in Enzymology, Vol. 182, Simon, M. I., ed., Academic Press, San Diego, Calif. (1990).

Many insect cell proteins are naturally rich in histidines, with some containing stretches of six histidines. When using the ProBond™ Purification System or other similar products to purify 6×His-tagged polypeptides, these histidine-rich polypeptides may co-purify with a polypeptide of interest. The contamination can be significant if the polypeptide of interest is expressed at low levels. 5 mM imidazole may be added to the binding buffer prior to addition of the polypeptide mixture to the column. Addition of imidazole may help to reduce background contamination by preventing polypeptides with low specificity from binding to the metal-chelating resin.

If the polypeptide of interest is 6×His-tagged and expressed intracellularly, the cells may be lysed and the lysate added directly to the ProBond™ column. 5×10⁶ to 1×10⁷ cells may be used for purification of a polypeptide of interest on a 2 ml ProBond™ column (see ProBond™ Purification System manual, catalog nos. R801-01, R801-15, version F, Invitrogen Corporation, Carlsbad, Calif.).

A suitable protocol is to seed 2×10⁶ cells in two or three 25 cm² flasks, grow the cells in selective medium until they reach confluence (4×10⁶ cells); wash cells once with PBS (Phosphate Buffered Saline, pH 7.4; Invitrogen Corporation, Carlsbad, Calif. Catalog no. 10010-023); harvest the cells by sloughing; transfer the cells to a sterile centrifuge tube; and centrifuge the cells at 1000×g for 5 minutes. The cells may be lysed immediately or frozen in liquid nitrogen and store at −80° C. until needed.

Many protocols are suitable for purifying polypeptides from the medium. The choice of protocol depends on the nature of the polypeptide being purified. The culture volume needed to purify sufficient quantities of polypeptide is dependent on the expression level of the polypeptide and the method of detection. One skilled in the art can develop suitable purification protocols using routine experimentation.

### Example 6

### Construction of Recombinant Baculoviruses

Baculoviruses have been extremely useful tools for heterologous expression of proteins in insect cells. Improved methods for cloning genes into baculoviral genomes (e.g., the 134 kb AcMNPV genome) have greatly simplified the process of recombinant baculovirus construction; however obtaining a purified viral stock still requires plaque purification and a minimum of 10-14 days. Current methods rely on recombination in insect or bacterial cells and are not well adapted for high-throughput experiments. To meet these challenges, materials and methods of the invention permit the construction of recombinant baculovirus in vitro. The recombinant baculovirus may be transfected directly into insect cells to generate the baculovirus stock.

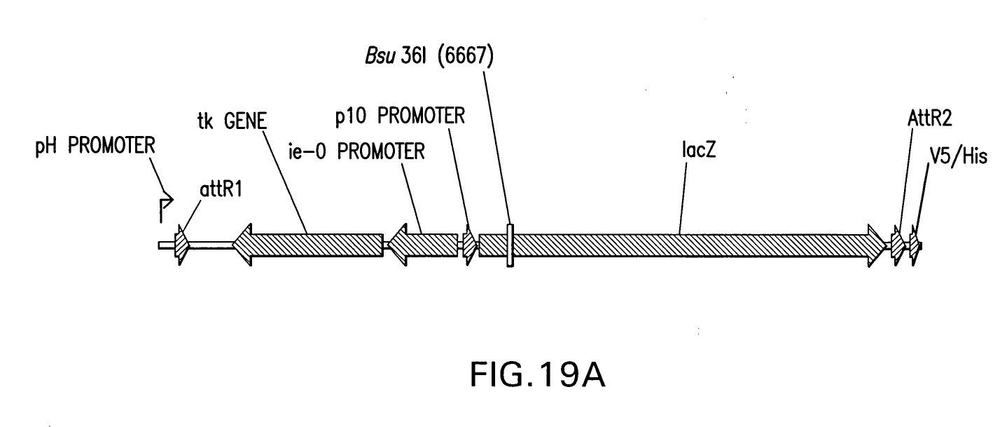

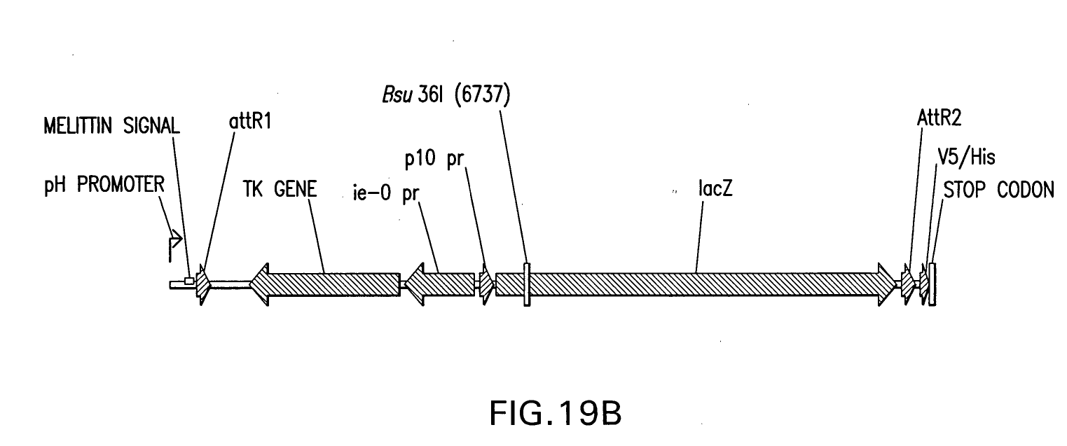

A baculovirus genome containing a recombination cassette (DEST) bounded by attR recombination sites compatible with GATEWAY™ entry vectors (Invitrogen Corporation, Carlsbad, Calif.) was constructed. Two transposition cassettes were constructed one with and one without the mellitin leader sequence. A schematic representation of the cassette without the mellitin sequence is provided in FIG. 19A and the sequence is provided in Table 13. A schematic representation of the cassette with the mellitin sequence is provided in FIG. 19B and the sequence is provided in Table 14. The DEST cassettes contain the HSV thymidine kinase (TK) gene driven by an immediate early promoter (IE-0 promoter) and the lacZ gene driven by a late promoter (P10 promoter). The genes permit identification of non-recombinant virus using a blue white screening protocol and selection against non-recombinant viruses using ganciclovir. The cassettes also contain the V5 epitope and a 6-Histidine sequence outside the attR2 recombination site. The sequence of the cassette contains a recognition site for the restriction enzyme Bsu36I (and its isoschizomer AocI) that is used to linearize the viral genome.

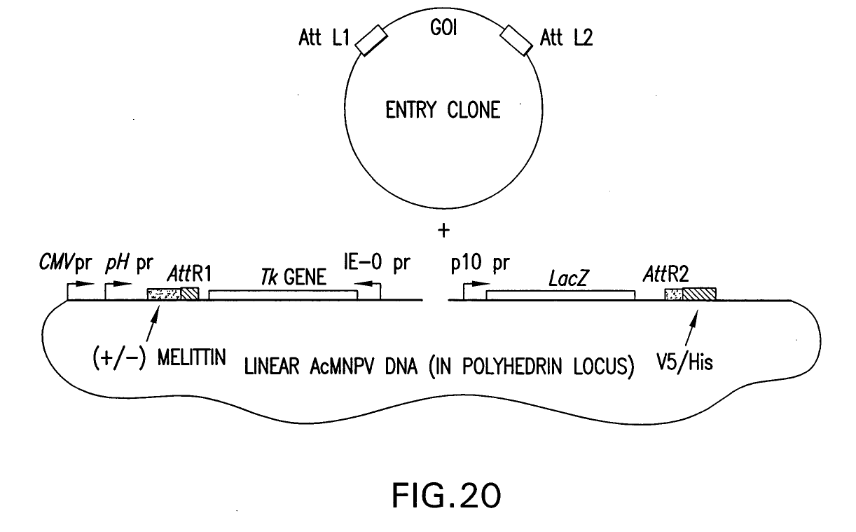

The cassette may be inserted into a baculoviral genome such that a sequence of interest in the Entry Clone may be operably linked to a baculoviral promoter (e.g., the polyhedrin promoter (ph pr in FIG. 20)) upon insertion of the sequence of interest into the viral genome. In practice, any eukaryotic cellular or viral promoter can be used to express a gene introduced from an entry clone, e.g. promoters from any of the above named baculovirus species, whether they are early, late, or very late. Although depicted as a gene sequence in FIG. 20, any sequence of interest may be inserted; the present invention is not limited to sequences encoding polypeptides.

In one embodiment, the nucleic acid sequence of interest may be recombined directly into the baculovirus genome downstream of the polyhedrin promoter, replacing the TK and lacZ genes. With reference to FIG. 20, the linearized baculoviral genome is depicted as a gapped circle. In the presence of the appropriate recombination proteins, the recombination sites (e.g., attR1 and attR2 sites) on the baculoviral genome will recombine with the recombination sites (e.g., attL1 and attL2 sites) on the nucleic acid molecule comprising the sequence of interest (Entry Clone in FIG. 20) resulting in recircularization of the baculoviral genome. The recombination reaction results in the transfer of the sequence of interest (depicted as a gene of interest (GOI) in FIG. 20) into the baculoviral genome. The transfer also results in the excision of the portions of the baculoviral genome between the attR recombination sites.

The resultant DNA may be directly transfected into insect cells to produce the recombinant viral stock. When the cells are grown on ganciclovir, only recombinant virus is able to replicate; replication of parental virus is prevented because of the TK gene product. The destination cassette may also be placed under the control of the CMV promoter or other promoter active in mammalian cells, for the purpose of transducing mammalian cells using baculovirus.

To demonstrate the feasibility of this system and to optimize conditions, the GFP coding sequence was first cloned into a nucleic acid molecule between two recombination sites and then transferred using recombinational cloning into a baculovirus genome comprising two compatible recombination sites. Sf21 cells were transfected with the recombination reaction mixture. After three days, the media from these cells containing budded virus produced from the first rounds of replication was used to infect a second population of cells, this time grown under ganciclovir selection. After 4 days, these cells were examined for GFP fluorescence and stained for LacZ expression. Cells infected by recombinant virus expressing GFP were fluorescent, while cells infected with remaining parental virus stained positive for LacZ expression. Using this assay method, conditions for transfection and ganciclovir counter selection were optimized. Under ideal conditions, small scale virus stocks essentially free of parental virus were produced within 7 days post-transfection. These stocks are suitable for creation of high titer stocks or further expression studies.

The utility of this system was then demonstrated for use in a 96 well format with collections of genes cloned into an Entry vector. Multiple genes in 96 well plates were cloned and screened for expression in parallel. Within seven days, purified viral stocks were available for scale-up or further expression studies.

In some embodiments, the present invention provides a new method for baculovirus cloning based on lambda recombination that is faster, requires less hands-on time, is more reliable, and is suitable for high throughput expression in 96 well plates.

In some embodiments, the present invention provides isolated nucleic acids comprising nucleic acid sequences that function as promoters. Optionally, the nucleic acid molecules may comprise one or more sequences of interest (e.g., ORFs, etc.) operably linked to one or more of the nucleic acid sequences that function as promoters. These promoters may function in any cell type, for example, mammalian, insect, etc.

In some embodiments, the promoters are tightly regulated. For example, in some embodiments, the promoters are not active unless one or more transactivators are present. In some embodiments, the nucleic acid sequences that function as promoters include, but are not limited to, the AcMNPV ORF 25 promoter sequence, the AcMNPV lef 3 promoter sequence, the AcMNPV TLP promoter sequence, the AcMNPV homologous repeat 5 sequence, other baculovirus homologous repeat sequences, and the like. The nucleic acid sequences of the AcMNPV ORF 25 promoter sequence, the AcMNPV lef 3 promoter sequence, the AcMNPV TLP promoter sequence, and the AcMNPV homologous repeat 5 sequence are provided in Table 15.

In some embodiments, the promoters discussed above are not active unless one or more transactivators are present. One suitable transactivator is the baculoviral IE-1 protein. The IE-1 promoter sequence, coding sequence, and polypeptide sequence are provided in Table 16. The transactivator may be provided on the same nucleic acid molecule comprising the promoter sequence or on another nucleic acid molecule (e.g., plasmid, virus, host cell genome, etc.). In some embodiments, the promoter sequence operably linked to a sequence of interest may be on one nucleic acid molecule (e.g. a plasmid) and the transactivator sequence may be on a different nucleic acid molecule (e.g., a virus such as a baculovirus). The nucleic acid molecule comprising the promoter sequence operably linked to a sequence of interest may be introduced into a host cell, for example, by transfection. The sequence of interest is not expressed or is substantially not expressed in the absence of a transactivator. In some embodiments, the host cell may be a eukaryotic cell, for example, a mammalian cell or an insect cell. The host cell comprising the nucleic acid molecule comprising the promoter sequence operably linked to a sequence of interest may be further contacted with a second nucleic acid molecule comprising the a sequence encoding the transactivator. Upon expression of the transactivator, the sequence of interest is expressed. In some embodiments, the transactivator polypeptide may be directly transfected into cells comprising the nucleic acid molecule comprising the promoter sequence operably linked to a sequence of interest. Such transactivator polypeptides may be present as native polypeptides or as fusion polypeptides, for example, as fusions with the herpesvirus VP22 polypeptide.

Nucleic acid molecules comprising the promoters discussed above may be used to conditionally express any sequence of interest. In some embodiments, the sequence of interest may encode a toxic polypeptide.

In some embodiments, nucleic acid molecules comprising the promoter sequences described above may have a homologous repeat (hr) sequence in cis with the promoter. Such homologous repeat sequences may be required for hr-dependent IE-1 transactivation.

The sequences provided in Table 15 are capable of functioning as conditionally activated promoters. The present invention also comprises portions of the sequences of Table 15 that function as conditionally active promoters. Such promoters may be activated by the IE-1 polypeptide. Such portions may comprise at least 50%, 60%, 70%, 80%, 90%, 95%, or more of one or more of the sequences in Table 15.

### Example 7

In some embodiments, materials and methods of the invention may be used to create stable cell lines expressing a nucleic acid sequence of interest. One non-limiting example is the InsectSelect™ system (Invitrogen Corporation, Carlsbad, Calif.), which is a stable insect cell expression system that utilizes a single plasmid for expression and selection. Nucleic acid molecules of the invention (e.g., InsectSelect™ vectors) may utilize different baculovirus immediate early promoters for expression of a sequence of interest and a selectable marker. Nucleic acid molecules of the invention may be constructed to be used in recombinational cloning methods. For example, pIB/V5-His (catalog no. V802001, Invitrogen Corporation, Carlsbad, Calif.) has been modified for using in methods involving recombinational cloning (e.g., GATEWAY™ cloning). In the modified vector, a different promoter is used to drive transcription of the blasticidin resistance gene than the OpIE-1 promoter used in pIB/V5-HIS.

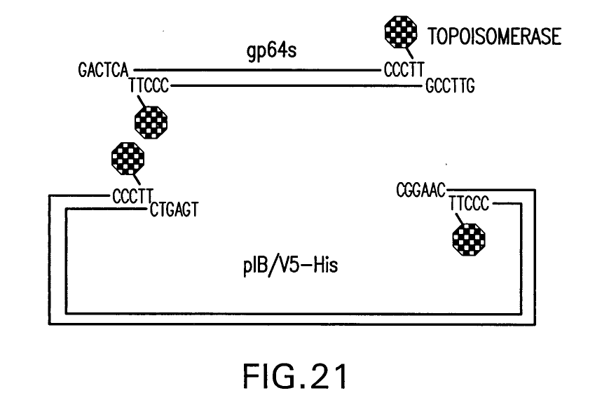

The OpIE-1 promoter was replaced with long or short versions of AcMNPVgp64 or pe38 promoters, using a Topoisomerase I mediated ligation strategy (FIG. 21). The AcMNPV gp64 and pe38 promoters were amplified from cosmid #58 (comprising AcMNPV bases 99803-132856 from a cosmid library of the AcMNPV genome, Harwood et al. Virology. 250:113-134, 1998) with promoter-specific primers that were appended at their 5′ ends with antisense TOPO sites and six additional bases (FIG. 21). pIB/V5-His was amplified with primers that included an anti-sense topoisomerase site and a six base sequence that becomes an overhang following topoisomerase binding. Each promoter (gp64s is illustrated) was amplified with similarly designed primers. Following binding, the overhangs annealed and were ligated by the enzyme. The oligonucleotide sequences are given below. The antisense topoisomerase sites are underlined.

17852 pIB Neg For
TGAGTCAAGGGCTGCCGGGCTGCAGCACTG
17853 pIB Neg Rev
CGGAACAAGGGCATGACCAAAATCCCTTAACG
17849 gp64 For
GACTCAAAGGGCTTGCTTGTGTGTTCCTTATTG
17850 gp64s Rev
GTTCCGAAGGGTTGTGTCACGTAGGCCAGATAAC
17851 gp64L Rev
GTTCCGAAGGGAATAATCGATTTAAGGGTGTAATACTC
17857 pe38 For
GACTCAAAGGGTTTGCTTATTGGCAGGCTCTCC
17858 pe38s Rev
GTTCCGAAGGGTATCTGTCCCCCACTCAGGC
17859 pe38L Rev
GTTCCGAAGGGTAAAGTTGATGCGGCGACGGC

The pIB/V5 His backbone was amplified using similarly designed primers. The PCR products were purified by gel electrophoresis and SNAP mini-prep columns. Following DpnI treatment to eliminate residual template vector, the PCR products were repurified by SNAP minipreps, eluted in 30 μl water and joined using topoisomerase (FIG. 21). Topoisomerase reactions were incubated at room temperature for 10 min and contained 8 μl of each PCR product, 50 mM Tris, pH 7.5, 0.1 μg/μl enzyme in 20 μl total volume. TOP10 E. Coli were transformed with the joined PCR products. Following selection on ampicillin plates, resulting colonies were grown overnight, and plasmid DNA isolated by miniprep (SNAP). The presence of the promoters was confirmed by restriction digest analysis. The construct containing gp64s was ultimately chosen for GATEWAY™ adaptation (see below).

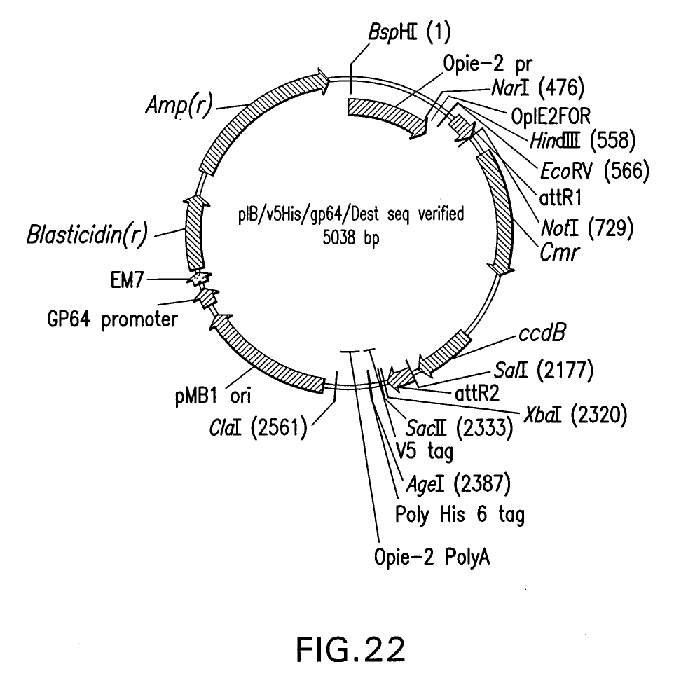

pIB/V5-His gp64 was modified to comprise recombination sites (i.e., GATEWAY™ adapted) by cloning a HindIII/XbaI fragment from pDEST38 into pIB/V5-His gp64, cut with the same enzymes. The vector was fully sequenced. A plasmid map is provided (FIG. 22).

To test the modified vector in a recombinational cloning reaction, pIB/V5-His gp64Dest was used for LR reactions with attL entry vectors containing LacZ, Calmodulin, TFIIS, and Apolipoprotein. The protocol used differed slightly from the protocol suggested in the GATEWAY™ manuals. The reaction conditions used were as follows:

2 μL LR clonase enzyme mix (catalog no. 11791043, Invitrogen Corporation, Carlsbad, Calif.)

2 μL LR reaction buffer

1 μL pENTR clone (˜300 ng DNA)

1 μL pDEST vector (˜300 ng DNA)

4 μL 0.5 M Tris buffer (pH 7.5)

Recombination reactions were incubated for 3 h at room temperature.

Reactions were not proteinase K treated. 2 μl of each recombination reaction was used to transform 50 μl TOP10 chemically competent bacteria. Half of the transformation mix was plated and yielded an average of 230 colonies. Thus, approximately 8000 colonies were obtained per μg entry vector. Colonies were grown in LB/Amp overnight and DNA was isolated by SNAP miniprep.

Experiments were performed with Sf21 cells or HighFive cells in serum-containing or serum free media (SFM). Grace's supplemented media with 10% FBS was used for both Sf21 and HighFive cells. For SFM treatments, Sf900II or ExpressFive media were used for Sf21 or HighFive cells, respectively. Twenty-four well plates were seeded with 1.8×10⁵ cells per well, and after 1 h attachment, washed with Grace's unsupplemented media. Transfection mixes contained 0.2 μg DNA and 1 μl Cellfectin® in 40 μl Grace's unsupplemented media and incubated for 30 min at RT. The transfection mixture was then diluted to 200 μl final volume in Grace's unsupplemented media and added to each well. Cells and transfection mix were incubated for 5 h with gentle rocking after which the mix was replaced with the appropriate media as described above. 48 h later the media was replaced with the same media containing between 10 and 25 μg/μl blasticidin, depending on the experiment. Cells used from stable cultures were under selection for at least 7 days. Cells were split as needed to maintain log-phase growth. Typically, 10 μg/ml blasticidin may be used for general purposes. However, one skilled in the art can optimize selection parameters for each construct using only routine experimentation.

Protein expression was monitored by western blot or LacZ activity assays. Cells from six well plates (approximately 10⁶ per well) were washed 2× in PBS, transferred to 1.7 ml tubes, spun down, resuspended in 500 μl lysis buffer (Tropix Galacto light kit, catalog no. T1006, Applied Biosystems, Foster City, Calif.), and then subjected to two freeze-thaw cycles. Lysates were microfuged at 16,000×g for 5 min. Supernatants were stored at −20° C. until used. Lysate protein concentration was measured using the BioRad protein assay against BSA as a standard. Various amounts of protein were denatured in LDS sample buffer (catalog no. NP0008, Invitrogen Corporation, Carlsbad, Calif.) and loaded on 4-12% NuPAGE gels (Invitrogen Corporation, Carlsbad, Calif.). Following electrophoresis, proteins were transferred to PVDF. The Western Breeze kit (catalog no. WB7104, Invitrogen Corporation, Carlsbad, Calif.) was used to visualize protein bands using anti-V5 coupled alkaline phosphatase at a 1:5000 dilution unless noted otherwise.

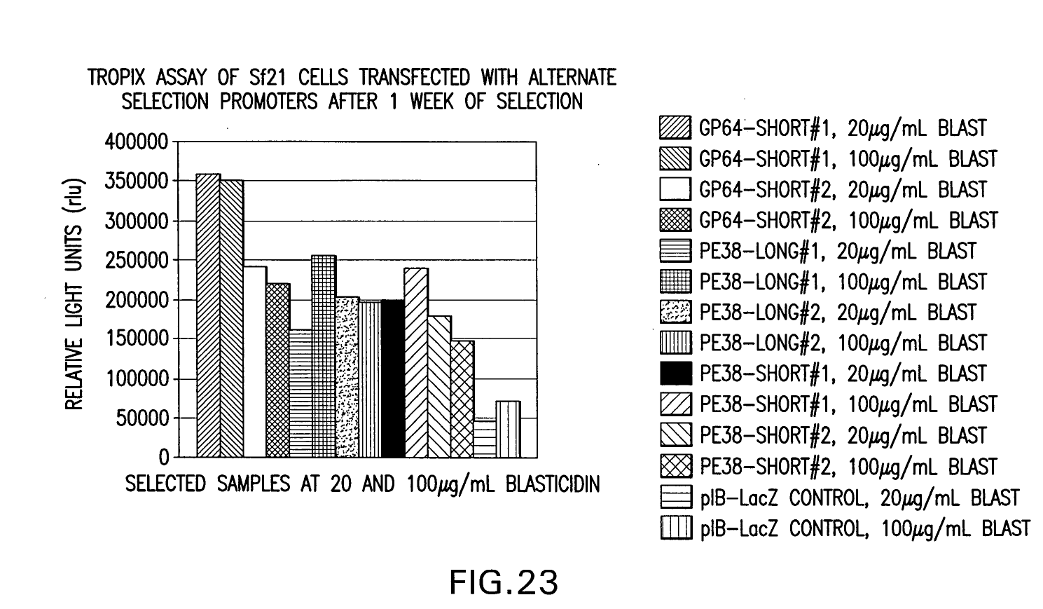

Without being bound by theory, is was though that use of a weaker promoter to drive antibiotic resistance would result in stable cultures that expressed the gene of interest at higher levels because the bsd gene (blasticidin resistance gene) was expressed at a lower level, integration of the plasmid containing the bsd gene would occur in more loci or in loci that were transcriptionally more active. Transcription of many baculovirus genes has been characterized, and suitable promoters were selected. The gp64 and pe38 promoters have both been extensively studied (Friesen, Regulation of baculovirus early gene expression, p. 141-170. In The Baculoviruses. L. K. Miller (ed.), Plenum Press, New York., 1997). The pe38 promoter is an immediate early promoter and thus does not require baculovirus infection for its activity. The gp64 promoter is transactivated by IE-1 but retains basal levels of activity without transactivation (Blissard, J. Virol. 65:5820-5827, 1991, Blissard, Virology. 190:783-793, 1992). The sequences responsible for IE-1 transactivation have been identified and are separable from the basal promoter (Blissard, 1992). A long (500 bp upstream of the ATG) and a short version (100 bp upstream of the ATG) for each promoter were obtained and cloned in place of the OpIE-1 promoter using TOPO-mediated ligation. LacZ was cloned into the resulting vectors. These constructs together with the OpIE1 promoter version of pIB LacZ/V5-His were transfected into Sf21 cells and polyclonal cultures were selected at two different dosages of blasticidin. The longer gp64 construct apparently did not provide sufficient levels of bsd expression and the cells died with the control cells. Surviving stable cultures were obtained from the other four constructs. Cells were harvested after two weeks of selection and expression levels were measured using β-galactosidase assays (FIG. 23). β-galactosidase activities for stable cell cultures established with different versions of pIB/V5-His. 20 μg of protein was used per assay. Higher levels of expression were obtained for all three alternate promoters than obtained with the OpIE-1 promoter at both 20 and 100 μg/ml blasticidin. There were not clear differences in LacZ activity between cultures selected at either concentration of blasticidin.

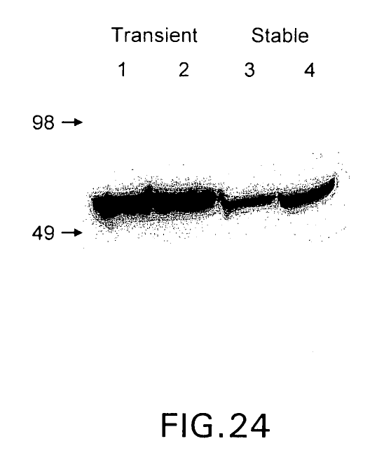

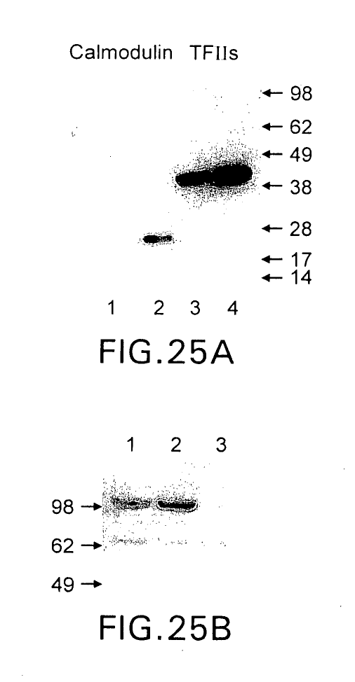

The gp64s promoter construct was used for GATEWAY™ adaptation. To examine the cloning efficiency and gene expression for the gp64s GATEWAY™ adapted version of this vector, four genes (Apolipoprotein, Calmodulin, TFIIs, and LacZ) were transferred into GATEWAY™ adapted versions of pIB/V5-His and pIB/V5-His gp64 the vector using an LR reaction. All LR reactions resulted in thousands of colonies per μg plasmid and were correct when examined by agarose gel electrophoresis. Each construct was transfected into Sf21 cells. Transient and stable expression of Apolipoprotein was compared between the gp64 and OPIE-1 versions of pIB Apolipoprotein/V5-His GATEWAY™. Transient expression levels were equivalent between the gp64 and OpIE1 versions (FIG. 24, lanes 1 and 2), but expression was higher for the gp64 version following selection (FIG. 24, lanes 3 and 4). To be sure that the higher stable expression level observed for the gp64 promoter was a general phenomenon, expression of Calmodulin, TFIIS, and LacZ between gp64 and OpIE-1 versions of pIB/V5-His GATEWAY™ were compared (FIG. 25). FIG. 25A shows expression of calmodulin and TFIIs from Sf21 cells stably transfected with OpIE-1 (lanes 1 and 3) and gp64s versions of pIB/V5-His. 8.6 μg total protein was loaded per lane. FIG. 25B shows expression of LacZ from Sf21 cells stably transfected with OpIE-1 (lane 1) or gp64s (lane 2) versions of pIB/V5-His. Lane 3 is a non-transfected control. 5.7 μg of protein was loaded per lane. As for Apolipoprotein, expression of Calmodulin, TFIIS (FIG. 25A) and LacZ (FIG. 25B) was higher from the gp64 version.

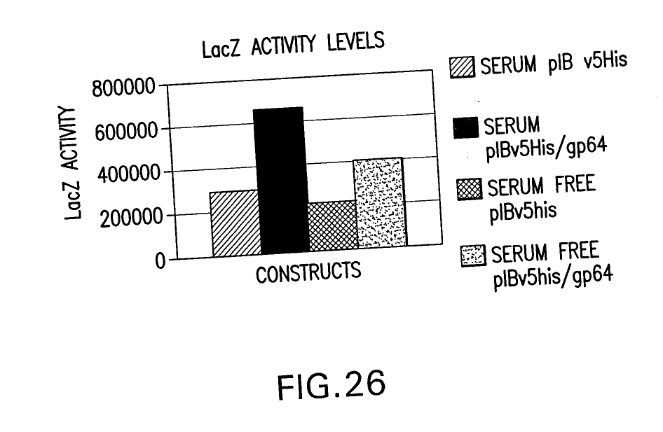

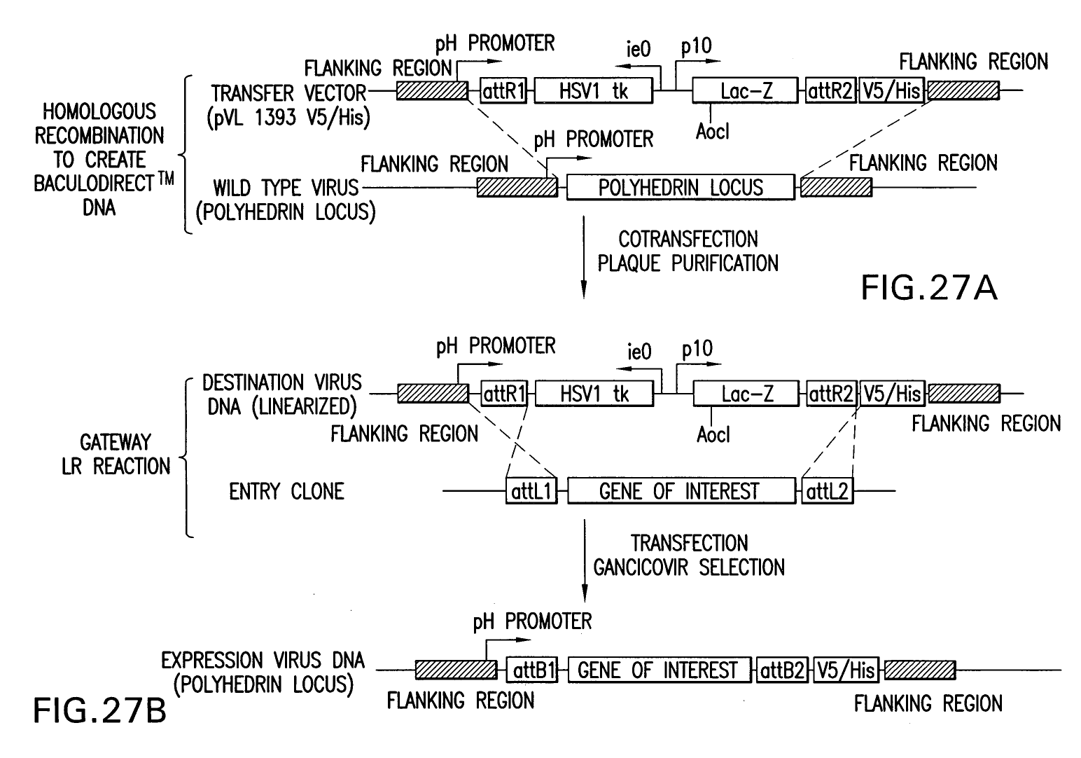

The above experiments were conducted with Sf21 cells in serum containing media. Use of a different promoter for expression of the antibiotic resistance marker could alter the dynamics of selection as a function of cell type or media used. Selection and expression from HighFive cells in serum- and serum free media was analyzed. In general, non-transfected cells were dead within a week but cells selected in SFM tended to die sooner (3-4 days) than those selected in media containing serum. As with the previous experiments, higher levels of gene expression were obtained from the gp64 construct with stably transfected HighFive cells, whether they were grown in serum or serum free media. Similar results were obtained with Sf21 cells in SFM media. FIG. 26 shows High five cells grown in serum and serum free media transfected with Gp64 and OpIE-1 versions of pIB/V5-His. 24.5 μg total protein per assay.

A recombinational cloning adapted version of pIB/V5-His that utilizes a different baculovirus promoter for expression of the bsd gene has been prepared. The basal gp64 promoter presumably results in lower levels of the bsd gene product than the OpIE-1 promoter used in pIB/V5-His and forces integration of the plasmid into more active chromosomal loci and/or at higher copy number.

### Example 8

In some embodiments, the present invention provides a method of making recombinant viruses using recombinational cloning. One non-limiting example is termed BaculoDirect™. Methods of this type provide a novel baculovirus cloning method that takes advantage of recombinational cloning technology (e.g., GATEWAY™ cloning technology, Invitrogen Corporation, Carlsbad, Calif.). With BaculoDirect™, an entry clone containing a nucleic acid sequence of interest (e.g., a sequence comprising a gene of interest) may be recombined into recombination-site-containing baculovirus genome in a one hour, in vitro reaction. The DNA product from this reaction can be transfected directly into suitable cells (e.g., Sf9 or Sf21 cells) to generate recombinant viruses and screen for expression. The ability to clone the sequence of interest (e.g., gene of interest (GOI)) directly into the baculovirus genome in vitro contrasts with existing baculovirus cloning methods in which the recombination step is performed in insect cells or bacteria. Compared with these existing baculovirus technologies, BaculoDirect™ is significantly faster, requires less hands-on time, and is more reliable. It is also easily adapted for high-throughput experiments. Thus, BaculoDirect™ offers significant advantages over current baculovirus cloning systems.

Throughout this disclosure, the term gene of interest (GOI) may be used for the sake of convenience. This should not be construed as limiting the present invention to nucleic acid sequences comprising genes. Any nucleic acid sequence of interest can be inserted into a vector of the invention using materials and methods described herein.

### Introduction

Baculoviruses are one of the most commonly used tools for eukaryotic expression of heterologous proteins. Traditionally, a GOI had to be first cloned into a transfer vector and then moved into the virus by homologous recombination into the polyhedrin locus in permissive insect cells. This occurred at low frequency. Plaque assays were tedious and required identification of polyhedrin negative plaques from among much more numerous polyhedrin-positive plaques.

During the last 20 years, innovations have made baculovirus cloning more convenient. Use of linearized DNA and design of the recombination strategy such that recombination restored function of an essential baculovirus gene boosted the proportion of recombinant plaques obtained from 1-2% to over 90% (Kitts and Possee. 1993. BioTechniques 14:810-817). However, multiple rounds of plaque purification were still required and the entire process of obtaining a useful viral stock took 3-4 weeks and a substantial amount of labor. Expression kits that use this technology are marketed by BD Biosciences Pharmingen, San Diego, Calif. (Baculogold™), Novagen Inc., Madison, Wis. and Invitrogen Corporation, Carlsbad, Calif. (Bac-n-Blue™).

A second method for baculovirus cloning utilizes site-specific recombination in bacteria to introduce the GOI into the baculovirus DNA (Luckow, et al., 1993. J. Virol. 67:4566-4579). The GOI is cloned into a transfer plasmid and used to transform a specialized bacterial strain that contains the baculovirus genome propagated as an F′ plasmid (bacmid). The GOI is then introduced into the bacmid by site-specific recombination between Tn7 sites on the transfer plasmid and in the baculovirus genome. Bacteria containing recombinant bacmids are then selected using antibiotic selection markers with appropriate selective media. The bacmid DNA is extracted and then transfected into insect cells. Plaque purification is, in theory, not required (except for the most rigorous applications) and the entire process from transfer plasmid to pure virus stock requires 10-12 days. Invitrogen Corporation, Carlsbad, Calif. markets this system under the trade name Bac to Bac™, catalog number 10359-016.

While these advancements in baculovirus cloning have greatly simplified use of baculovirus for routine protein expression, the methods described above still require significant “hands-on” time and are not well suited for parallel processing of multiple genes (i.e., high-throughput). The present invention provides a new method that greatly simplifies and shortens the process for cloning and purification of baculovirus recombinants. One non-limiting example of the present invention is BaculoDirect™, which utilizes GATEWAY™ recombinational cloning technology (Invitrogen Corporation, Carlsbad, Calif.) to recombine a GOI into the baculovirus genome in vitro in a one hour, room temperature reaction. The resulting recombinant virus DNA is transfected directly into insect cells. In just six days, cells can be harvested for expression screening to obtain a pure viral supernatant suitable for creation of high titer stocks.

### Materials and Methods

All materials used in this study were from Invitrogen Corporation, Carlsbad, Calif. except restriction enzymes (Roche Applied Sciences, Indianapolis, Ind. or NEB, Beverly, Mass.) and ganciclovir sodium salt (GCV, Invivogen, San Diego, Calif. Catalog #sud-gcv).

Cells and Virus

Sf21 cells were cultured in Grace's medium with supplements and 10% FBS unless stated otherwise. Infection of cells with wild type AcMNPV or other viruses was performed as described (O'Reilly et al., 1992. Baculovirus Expression Vectors: a Laboratory Manual. W. H. Freeman Co., New York).

Plasmid and Virus Construction

Three versions of BaculoDirect™ were constructed. The first contained the melittin secretion signal, the second contained both a melittin signal and a C-term V5/His tag, and the third had a C-term V5/His tag without a secretion signal. FIGS. 19A and 19B provide schematics of recombination cassettes having a C-terminal V5/His tag with (19B) and without (19A) a melittin leader.

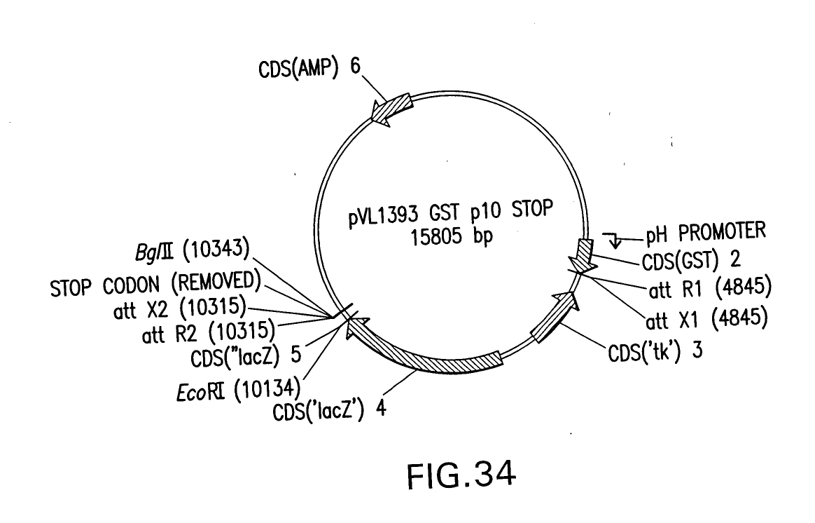

The plasmid pVL1393 GST p10 stop (FIG. 34) was digested with BamHI and NcoI. A 15 kb band was purified (removing the GST tag) to which was ligated, a double stranded oligonucleotide containing the melittin signal flanked by BamH1 and NcoI overhangs. The ligated products were transformed into TOP10 bacteria and the correct clones verified by restriction digestion and sequencing. This plasmid (pVL1393 Mel Stop) contained a stop codon downstream of the attR2 site that had to be removed by PCR directed site-specific mutagenesis. Primers EcoRI sense (GAA TTCCAGCTGAGCGCCGGTCGCTAC) and BglII antisense (AGATCTTCATTCATTCTCACCACTTTGTACAAG) were used to amplify a fragment from pVL1393 Mel Stop, and the resulting 209 bp fragment was cut with EcoRI and BglII, and then ligated to pVL1393 Mel Stop cut with the same enzymes. The correct clone was identified by restriction digestion and sequence analysis. This gave pVL1393 Mel no-Stop.

Next, a V5-His tag was added downstream of the attR2 site. The V5/His sequence was amplified from pIND/V5-His-TOPO (catalog no. K101001, Invitrogen Corporation, Carlsbad, Calif.) with primers containing BglII sites at each 5′ end (V5/His 5′: AGATCTGGGGAAGCCTATCCCTAACCC; V5/His 3′: AGATCTTCAATGGTGATGGTGATGATGACCGG). The amplicon was cloned into pCR2.1 TOPO TA and then removed by BglII digestion and ligated to pVL1393 Mel no-Stop cut with BglII. The correct clones were identified and verified by sequencing. This resulted in plasmid pVL1393 MeVV5-His. The melittin signal was subsequently removed by replacing the melittin-attR1 sequence from pVL1393 Mel/V5-His with the attR1 sequence from pVL1393-Native, using NotI and BamHI. The correct plasmid clones were verified by sequencing and dubbed pVL1393 V5/His. FIG. 27 shows a schematic of the strategy for construction of BaculoDirect™ DNA. In FIG. 27A, the GATEWAY™ counter selection cassette was cloned in the polyhedrin locus of wt AcMPNV by homologous recombination between with pVL1393 V5-His. The resulting virus DNA contains the counter selection cassette bounded by attR sites, immediately downstream of the polyhedrin promoter and upstream of the V5/His tag. In FIG. 27B, LR recombination between BaculoDirect™ DNA and an entry clone results in an expression virus in which the counter selection cassette is replaced by gene of interest.

Generation of BaculoDirect™ viruses

BaculoDirect™ viruses were created via conventional homologous recombination between wt AcMNPV and homologous recombination sequences contained in pVL1393 (FIG. 27, O'Reilly, et al., 1992). Briefly, Sf21 cells were co-transfected with 0.5 μg wild type AcMNPV E2 virus DNA and 3-5 μg of pVL1393 V5/His. After five days, the supernatant was collected. This supernatant contained a mixture of recombinant BaculoDirect™ virus and wt virus. The recombinant virus was isolated and purified through three to four rounds of plaque purification (O'Reilly, et al., 1992). Recombinant plaques could be distinguished from wt by phenotype, i.e., recombinant plaques were β-Gal⁺, polyhedra(−) whereas wt plaques were β-Gal(−), polyhedra(+).

### Generation of Recombinant Expression Virus

Expression viruses were generated by performing standard LR clonase reactions between BaculoDirect™ DNA and entry clones containing a GOI flanked by attL1 and attL2 (FIG. 27B, GATEWAY™ Instruction Manual Version C, 6/02, Invitrogen Corporation, Carlsbad, Calif.). Where indicated, BaculoDirect™ DNA was linearized using AocI (an isoschizomer of Bsu36I), which cuts in the 5′ end of the lacZ gene. Reactions were performed with or without linearization. Twenty microliter LR reactions contained 300 ng viral DNA, 100 ng entry clone, 4 μl LR clonase buffer, 4 μl LR clonase, and were incubated for 1 h at room temperature. Two million Sf21 cells were transfected with varying amounts of completed LR reaction using 6 μl of Cellfectin® (catalog no. 10362-010, Invitrogen Corporation, Carlsbad, Calif.) and Sf900II media per the manufacturer's instructions. Five hours post-transfection, transfection buffer was replaced with the Grace's Supplemented Insect Medium containing 10% FBS and 100 μM ganciclovir. Three to five days later, the supernatant was collected and varying amounts were used to infect fresh Sf21 cells with or without ganciclovir selection.

High Throughput (HTP) Screening of Expression

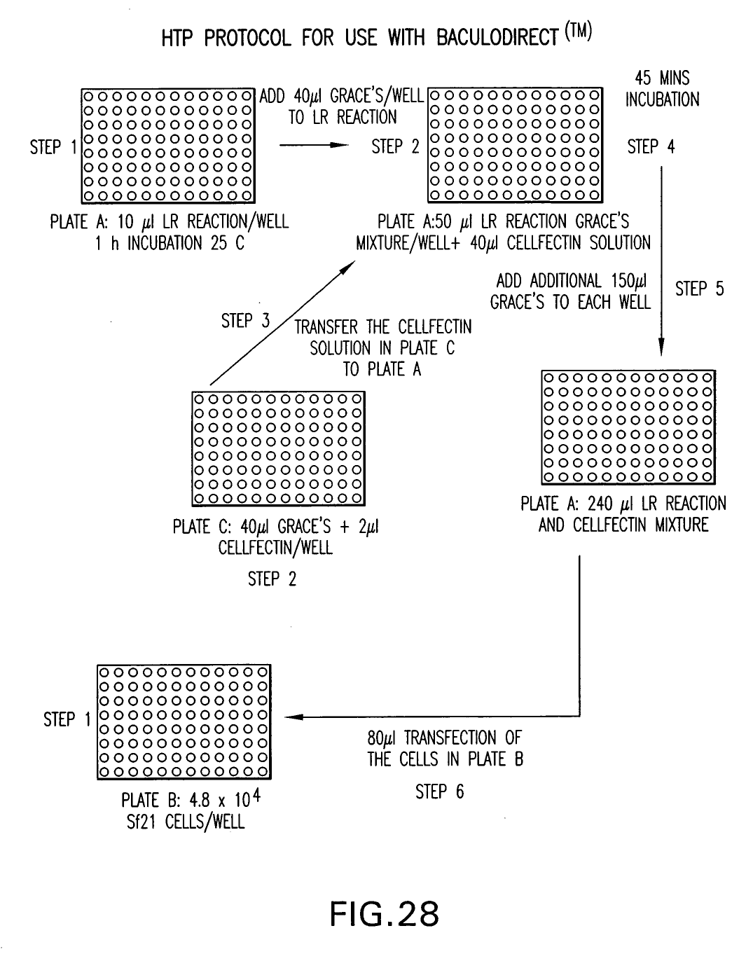

A method for performing LR reactions and transfection in 96 well plate format was developed. FIG. 28 provides a schematic illustration of BaculoDirect™ cloning and expression in 96 well plates. Entry vector DNAs, diluted Cellfectin®, and Sf21 cells were arrayed in 96 well plates. By arraying the components separately, the number of pipetting manipulations of the Baculovirus DNA is minimized. Following expression screening from the first generation transfection, only those wells showing expression of a protein of interest need be processed further.

Three 96 well plates were needed in this experiment. In plate A, 10 μl LR reactions were assembled in individual wells, starting with five different entry plasmids arrayed in multiple wells. The entry clones used were: pENTR APO/V5-His (Apolipoprotein), pENTR CAL/V5-His (Calmodulin), pENTR GUS, pENTR LacZ and pENTR CAT. Each 10 μl reaction included 50 ng entry clone, 150 ng purified linear BaculoDirect™ DNA, 2 μl LR clonase buffer, and 2 μl LR clonase. The LR reactions were incubated in the plates for 1 h at RT. During the LR incubation, Sf21 cells were seeded at 4.8×10⁴ cells per well in a separate plate and allowed to attach in plate B. In plate C, 2 μl of Cellfectin® were diluted to 40 μl per well with Grace's medium. After the 1 h LR reaction, 40 μl of Grace's unsupplemented media were added to each well of plate A. Forty microliters of the Cellfectin® mixture from plate C were added to the diluted LR reactions and incubated at 27° C. for 30-45 min. After this incubation, 150 μl of Grace's un-supplemented media was added to the wells of plate A. The cells in plate B were washed twice in Grace's media and then replaced with various amounts of the transfection mixture from plate A. Plate B was incubated for 5 h at 27° C., and then the transfection mixture was removed and replaced with Grace's complete media with 100 μM ganciclovir. The cells were allowed to grow for 3-4 days. Supernatants from each well were transferred to a separate plate. The cells remaining in plate A were lysed in situ with 100 μl LDS lysis buffer and heated to 80° C. for 5 min. Because apolipoprotein was secreted, 15 μl of supernatant was denatured in 4× sample buffer. Protein samples were separated on SDS-PAGE gels, transferred to PVDF and visualized by western blot.

### Estimation of Viral Titers

Virus titers were estimated using two methods. Virus plaque assays were performed using techniques well known in the art (e.g., Bac to Bac Baculovirus Expression System Manual, catalog no. 10359-016, version C, p. 27, Invitrogen Corporation, Carlsbad, Calif.). P1 or virus supernatants (infection from the P1 stock) using apolipoprotein-expressing versions of each virus were serially diluted ten fold from 10⁻¹ to 10⁻⁸ and used to infect 2 million cells in six well plates. Recombinant plaques were counted and titers estimated based on the dilution factor for each plate.

TCID₅₀ (Tissue Culture Infective Dose) measurements were conducted as described (O'Reilly, et al., 1992). Briefly, a 96 well plate was seeded with 4.8×10⁴ Sf21 cells per well. P1 stocks or virus supernatants were as described above. 10 μl of each dilution was added per well, twelve wells per dilution, using a multi-channel pipettor. The TCID₅₀ was calculated using the Excel (Microsoft) spreadsheet described in O'Reilly, et al., 1992.

### Results

### Optimization of LR Clonase Reactions Using Baculodirect™ DNA

BaculoDirect™ DNA is the functional equivalent of a GATEWAY™ destination vector. GATEWAY™ destination vectors designed for use in bacteria, e.g., E. coli, contain a counter-selection cassette containing the ccdB gene and a chloramphenicol resistance marker, bounded by attR sites. Recombination between an attL containing entry clone and the destination plasmid replaces the ccdB gene and Chl(r) marker with the gene of interest, yielding an expression clone bounded by attB sites. This selection scheme does not work in insect cells. To create a counter-selection cassette for use with baculovirus, wild type baculovirus DNA was engineered with a cassette containing the herpes virus TK gene (HSV tk) and the lacZ gene, both under control of baculovirus promoters, bounded by attR sites (FIG. 27A). The attR cassette was placed immediately downstream of the polyhedrin promoter. Recombination between the “destination virus” and an entry clone replaces the counter selection cassette with the GOI under polyhedrin promoter control (FIG. 27B). Transfection of the resulting DNA creates a mixed baculovirus infection with both recombinant virus and parent virus present. Replication of the parent virus is prevented by growing the cells in the presence of ganciclovir, which is metabolized by the HSV tk gene into a toxic inhibitor of DNA replication (Godeau, et al., 1992, Nucl. Acids Res. 20:6239-6246). Cells that are infected by parent virus will also express the lacZ gene, which can be assayed by staining infected cells, providing a method for checking the purity the virus infection.

To test if the LR reaction would work between a 3-4 kb entry clone and the 140 kb BaculoDirect™ virus DNA, an LR reaction between melittin BaculoDirect™ and a GFP entry clone was performed. GFP expression was clearly visible by fluorescence as early as 48 h post-transfection and was stronger at 72 h, demonstrating that the LR reactions were successful and that GFP was placed under control of the polyhedrin promoter. Transfection, infection and selection conditions were then optimized to minimize background resulting from residual parental virus, as evidenced by GFP fluorescence and β-galactosidase staining.

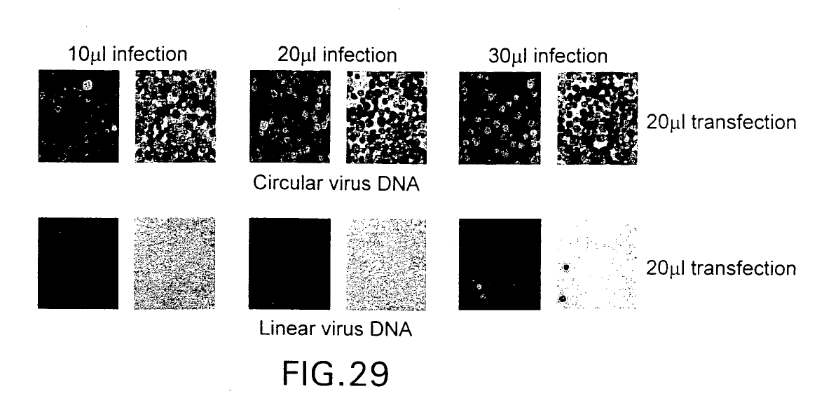

Linear and circular BaculoDirect™ DNA were compared. Thus, a standard LR reaction was performed with either linearized or circular (uncut) melittin BaculoDirect™ DNA, without ganciclovir selection. Ten, twenty or thirty microliters of LR reaction were used to transfect Sf21 cells (only the results from the 20 μl transfection are shown in FIG. 29). Three days later, varying amounts of supernatant (P1 stock from the “first generation”) from each transfection were used to infect new Sf21 cells. After four days, infected cells were examined for GFP fluorescence and then stained for β-galactosidase activity. These cells are from the “second generation” and the supernatant from them is a small scale high titer stock (see titer data below). Virtually all cells in all treatments were fluorescent, demonstrating that a productive baculovirus infection had been established and that the virus was actively expressing GFP. FIG. 29 shows the results of an analysis of cells transfected with LR reaction products from the melittin version of BaculoDirect™ DNA. LR reactions between melittin BaculoDirect™ DNA were performed with AocI cut or circular virus DNA and a GFP entry clone. Sf21 cells were transfected with 10 μl, 20 μl or 30 μl each LR reaction, using either linear virus DNA or circular virus DNA as indicated, without GCV selection. Cells were examined by fluorescence and β-Gal staining 72 hours following transfection. The result here shown from the 20 μl of LR reaction was typical. Some β-Gal positive cells were found in every well examined. β-galactosidase activity (i.e., background) was much higher in cells that had been infected with P1 virus derived from cells transfected with LR reactions that used circular rather than linearized BaculoDirect™ DNA (FIG. 29, upper panel). Background was much lower if cells were infected with P1 stocks derived from LR reactions with linearized BaculoDirect™ DNA, although some background was detected when more P1 stock was used for infection (FIG. 29, lower panel).

The effect of ganciclovir selection was then tested by growing cells in the presence or absence of ganciclovir. LR reactions were performed as above with circular or linearized melittin BaculoDirect™ DNA. SF21 cells were transfected with varying amounts of LR reaction and then grown without GCV (first generation). After 72 h, varying amounts P1 stock obtained from each transfection were used to infect new Sf21 cells, now grown in the presence of 100 μM GCV. After 4 days (second generation), the cells were examined for GFP fluorescence and stained for β-galactosidase. GCV did not appreciably reduce the number of cells staining positive for β-galactosidase activity when infections were derived from LR reactions using circular virus, whereas GCV reduced the number of β-gal positive cells from infections derived from LR reactions that used linearized virus.

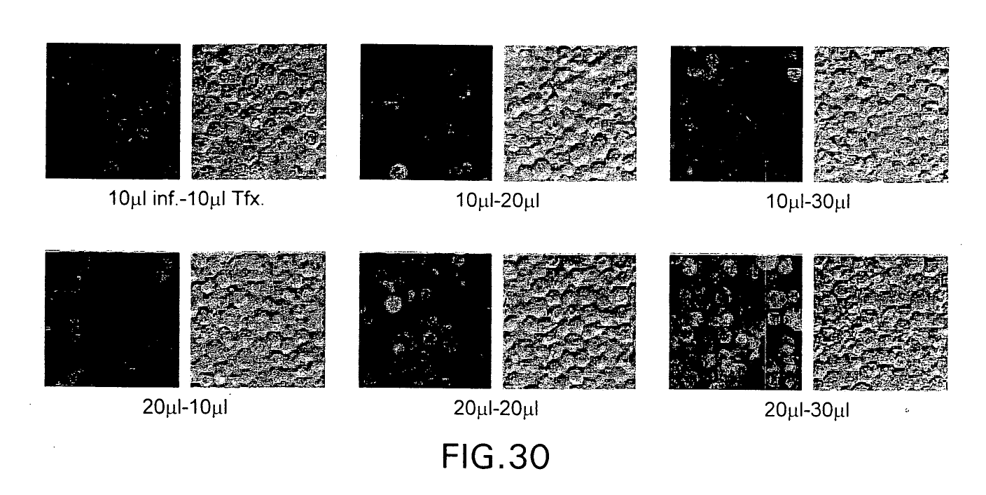

The effectiveness of ganciclovir in eliminating background when used during the first generation, the second generation or both generations was tested. When ganciclovir was used in the first generation or the second generation, at least some blue cells were observed following the second generation. In general, more background was found when more LR reaction was transfected. However, when ganciclovir was used in both generations, no blue cells were found, suggesting that there were no cells infected by parent virus following two rounds of ganciclovir selection. Moreover, zero background was found irrespective of how much LR reaction was used during for the transfection. Representative results from these experiments are shown in FIG. 30. In the experiment shown in FIG. 30, cells were transfected and selected during both generations as described above. Following the second generation, the cells were photographed to illustrate typical results following the selection protocol. Essentially all cells were producing GFP, but no cells stained positive for β-Gal if GCV selection was maintained during both generations. Thus, parent virus is not replicating in these cells. These results were obtained with cells grown in serum. The same result was found when serum free-adapted Sf21 cells were grown and selected with GCV in serum free media. Similar results were subsequently found using linearized V5/His virus.

### High-Throughput Screening of Expression

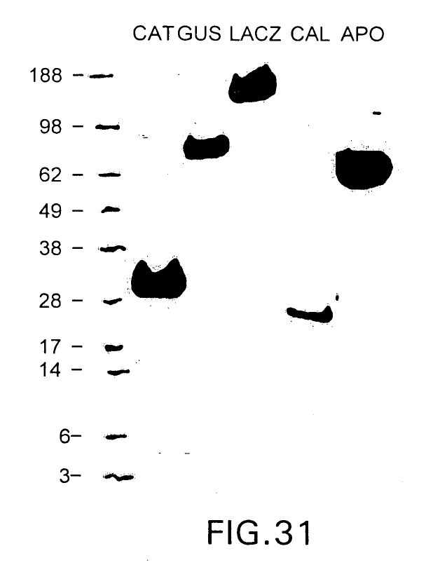

The ability to clone and express genes from baculovirus without plaque purification or selection in bacteria suggests that BaculoDirect™ can be used conveniently for high-throughput screening of expression. Five pENTR clones were chosen (CAT, GUS, LacZ, Apolipoprotein/V5-His, and Calmodulin/V5-His) for expression. Each pENTR DNA was arrayed in multiple wells of a 96 well plate as illustrated in FIG. 28. LR clonase reaction mixes were added as described in the Materials and Methods, using linearized V5/His BaculoDirect™ DNA. All manipulations used multi-channel or repeating pipettors and thus could also be performed robotically. Following ganciclovir selection during the first generation, expression from each virus was assayed by western blot. All five genes expressed at levels sufficient to be easily detected (FIG. 31). The supernatants were stored in a separate 96 well plate and were available for second round infection and selection.

FIG. 31 shows the results of the screening of protein expression from LR reactions performed in a 96 well plate. Indicated pENTR DNAs were arrayed in a 96 well plate, and LR reactions were performed as described above. Supernatants were removed to a separate plate and then cells were lysed using 100 μl LDS sample buffer. 15 μl of lysate was applied per lane except for apolipoprotein, which was secreted. For apolipoprotein, 11 μl of supernatant was used instead of cell lysate. The blot was visualized with anti V5:AP conjugate at 1:5000 and exposed to film for 15 sec.

Titer Comparison

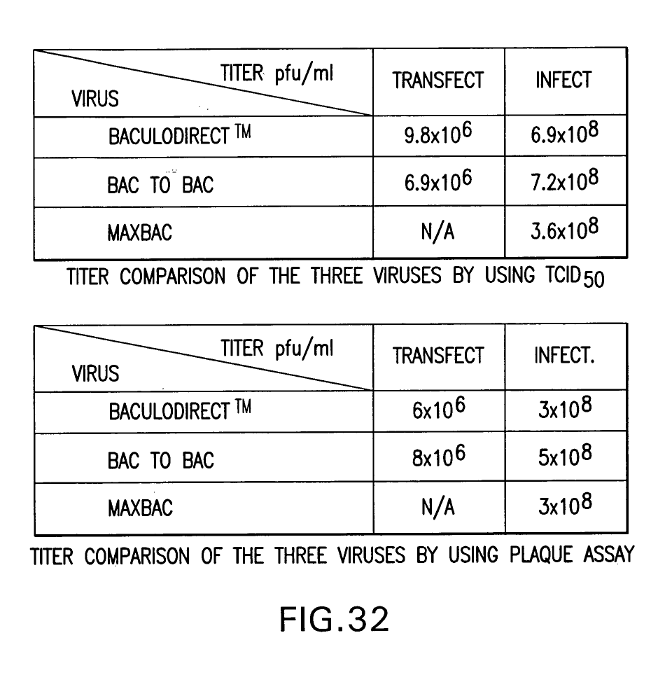

Two methods were used to compare the virus titers obtained from BaculoDirect™ with Bac to Bac™ or Bac-n-blue™. Apolipoprotein was cloned into pBlueBac 4.5/V5-His and co-transfected this with linear Bac-n-Blue DNA into Sf21 cells using well known techniques (e.g., Bac-n-blue™ manual, catalog no. K855-01, version M, Invitrogen Corporation, Carlsbad, Calif.). Following one round of plaque purification, a high titer stock was made. The entire apolipoprotein/V5-His reading frame was cloned into pFastBac, and bacmid DNA was generated using standard techniques (e.g., Bac to Bac™ manual, catalog nos. 11827-011, 11806-015, 11804-010 and 11807-03, version C, Invitrogen Corporation, Carlsbad, Calif.). The bacmid DNA was transfected into Sf21 cells, and a high titer stock was made. The apolipoprotein/V5-His reading frame was also cloned into pENTR and transferred in an LR reaction into linearized V5-His BaculoDirect™ DNA. Titers were measured following transfection and infection using plaque assay and TCID₅₀ methods. The titers obtained following infection were similar for all three baculovirus expression systems using either method and were in the range of 3×10⁸ to 7×10⁸ pfU/ml (FIG. 32). 10⁸ pfu/ml is a typical titer for baculovirus and thus BaculoDirect™ baculoviruses replicate as well as the baculoviruses used in other systems.

FIG. 32 shows an estimation of virus titers using plaque purification and TCID₅₀ measurements. Apolipoprotein was cloned into pENTR, pFASTBAC, or pBlueBac 4.5/V5-His (catalog no. V207520, Invitrogen Corporation, Carlsbad, Calif.). Procedures for MaxBac and Bac to Bac were followed as described in their respective instruction manuals. Dilutions of P1 or virus stock from second generation supernatants were serially diluted and used to infect cells for agar overlay (plaque purification) or in 96 well plates (TCID₅₀). For BaculoDirect™, cells were selected on 100 μM ganciclovir for both generations. Titers were calculated as described (O'Reilly, et al., 1992).

### Discussion

BaculoDirect™ is functionally a GATEWAY™ adaptation of the baculovirus genome. Lambda-based recombination occurs between the attR sites engineered in the baculovirus genome and attL sites surrounding the GOI in an entry clone. Following the LR clonase reaction, the counter-selection cassette containing the HSV tk gene and lacZ driven by baculovirus promoters and bounded on each side by attR sites on the baculovirus is replaced by the GOI from the entry clone. This results in re-circularization of the virus DNA. Replication of parent virus is prevented, both because it remains linearized, and because the tk gene product prevents DNA replication in the presence of ganciclovir. Linearization was highly effective at preventing replication of parental virus (FIG. 29). Virtually all cells expressed β-Gal following transfection and ganciclovir selection if the LR reaction was performed with circular virus, whereas use of linear virus boosted to greater than 95% (FIG. 29).

The presence of lacZ in the counter-selection cassette provides a means of judging the purity of virus stocks, since the absence of β-Gal cell staining is a good indication that a virus stock is free of contaminating parent virus.

Depending on the context, the attB2 site can sometimes pose a problem for expression and or detection from the C-terminal V5 epitope tag. In FIG. 31, APO and CAL were cloned without an internal attB2 site, while the remaining three genes were cloned with an internal attB2 site between the gene and the V5-His tag. The entry clones used for APO and CAL had an encoded C-terminal V5-His. All of the genes except for CAL appeared to be expressed and detected at high levels (FIG. 31). It has been observed that CAL tends express at lower levels in most experiments. BaculoDirect™ viruses that express GUS with or without attB2 inside the reading frame have been constructed. GUS expression was detected equally well for both versions, suggesting that the attB2 site does not appear to interfere with expression or detection from the V5 tag in the context in which it is used in BaculoDirect™.

The addition of the GATEWAY™ cassette, presence of attB sites in the polyhedrin locus, or ganciclovir selection, did not appear to affect virus titers when compared to other baculovirus expression systems (FIG. 32). Titers in excess of 10⁸ pfu/ml were obtained routinely following the second-generation virus infection. Since the virus stocks obtained following selection on ganciclovir were found to be essentially pure (based on the lack of infected cells that were β-Gal positive), the virus supernatants obtained at this stage require no plaque purification and can be scaled up for production of high titer stocks. The entire process, from LR cloning to pure virus stock can be performed for multiple genes simultaneously in 96 well plates. Although the present methodology has been exemplified using just five genes, one of skill in the art will appreciate that any number of genes (e.g., 20, 50, 100, 250, 500, 1,000, 2,500, 5,000, etc.) can be processed in a similar manner. The present method allows screening for expression after just three days, and continued selection and scale-up can focus on only those wells that express the desired protein product.

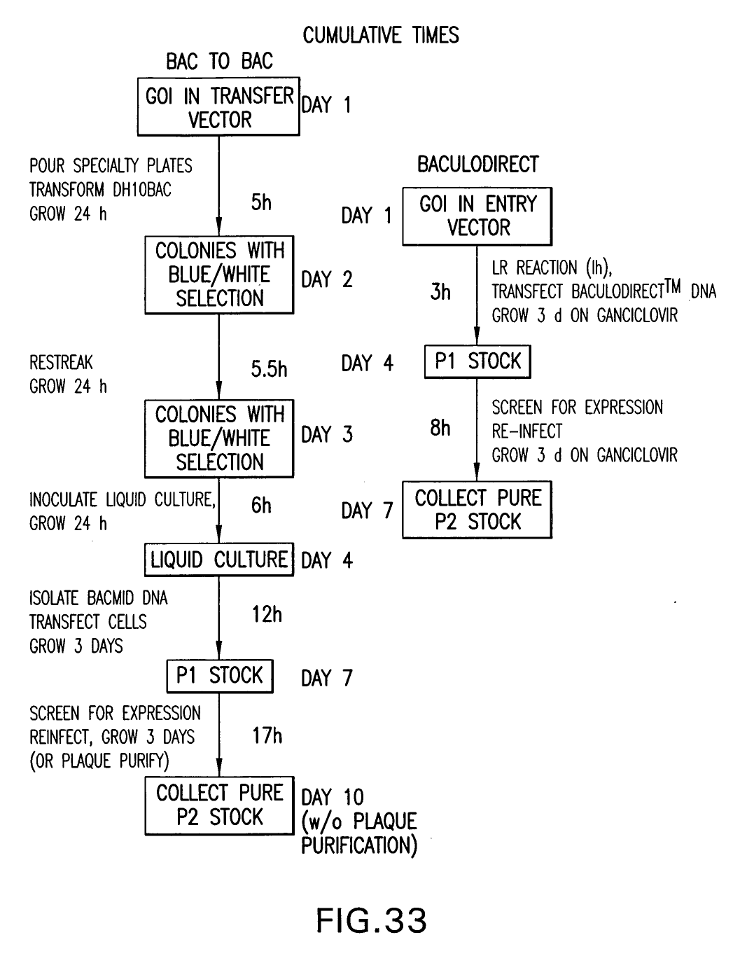

FIG. 33 shows a comparison of the time required for expression testing and virus purification between BaculoDirect™ and Bac to Bac. Numbers next to the arrows between steps are cumulative labor time in hours. Chronological elapsed times are indicated in days. Procedures common to both systems were given equal times, e.g., 2 hours for transfection, 4 hours for expression testing.

Compared to other baculovirus expression systems, the methods described herein (e.g., BaculoDirect™) require much less hands-on time and are faster chronologically. For example, Bac to Bac™ requires 10 days to obtain a purified viral stock and upwards of 17 hours of actual labor (FIG. 33) This assumes that the P1 stock obtained with Bac to Bac™ does not require plaque purification. In practice, one of skill in the art is likely to have difficulty in obtaining pure stock without plaque purification; as a result, plaque purification is now being encouraged for Bac to Bac™ users. The MaxBac baculovirus expression system relies on homologous recombination in insect cells, and, like other methods utilizing homologous recombination, requires plaque purification and even more chronological time and labor. By contrast, BaculoDirect requires only 8 hours of labor over six days to obtain a purified virus stock suitable for production of high titer stocks.

In summary, one of skill in the art with a collection of clones adapted for use in recombinational cloning methods (e.g., pENTR clones adapted for GATEWAY™ methods) will be able to clone and express their genes of interest quickly in a baculoviral expression system of the present invention, using simple protocols, and in parallel reactions.

A suitable protocol for production of the recombinant baculoviruses of the invention is as follows:

Materials: Sf9 or Sf21 cells growing in log phase; linearized BaculoDirect Virus DNA; GOI cloned into L1/L2 Entry clone (e.g., pENTR CAT; LR clonase Buffer; LR clonase; and ganciclovir sodium (100 mM solution in water).

A sequence of interest may be cloned into L1/L2 entry vector. Suitable cells (e.g., Sf9 or Sf21 cells) may be plated at recommended densities (e.g., Guide to Baculovirus Expression Systems and Insect Cell Culture, catalog nos. 10359016, 10360014, 10608016, 11827011, Invitrogen Corporation, Carlsbad, Calif., Feb. 27, 2002). HighFives are less preferred as they give low infectivity/titer. A suitable method may employ 6 well plates with 2 million Sf21 cells. An LR reaction may be performed between Entry vector and BaculoDirect™ linearized DNA (GATEWAY™ Manual) using 100 ng entry vector and 300 ng linearized BaculoDirect™ DNA. 1 h at room temperature. An aliquot (e.g., 10 μl) of LR reaction may be transfected into the cells (e.g., using Cellfectin® protocol). Transfection media may be replaced with growth media of choice, supplemented with 100 μM ganciclovir. After 72 hours, an aliquot (e.g., 10 μl) of supernatant from transfected cells can be added to fresh well of cells with 100 μM ganciclovir in growth medium. Protein expression can be checked by western blot at this time. After 72 hours, supernatant can be collected (e.g., in a sterile tube)x. Recommended: Stain cells with β-Gal staining kit. Viruses may be amplified as per standard protocols.

### Example 9

In some embodiments, the present invention provides materials and methods for the construction and use of recombinant retroviruses, e.g., lentiviruses. Although the present invention is exemplified using a lentivirus, any other type of retrovirus may be used in an analogous fashion to practice the present invention. A commercially available system for the construction of recombinant lentiviruses is ViraPower™ Lentiviral Expression System, available from Invitrogen Corporation, Carlsbad, Calif. The ViraPower™ system provides a retroviral system for high-level expression in dividing and non-dividing eukaryotic cells, e.g., mammalian cells. Examples of products available from Invitrogen Corporation, Carlsbad, Calif. include the ViraPower™ Lentiviral Directional TOPO® Expression Kit catalog number K4950-00, the ViraPower™ Lentiviral GATEWAY™ Expression Kit catalog number K4960-00, and the ViraPower™ Lentiviral Support Kit catalog number K4970-00.

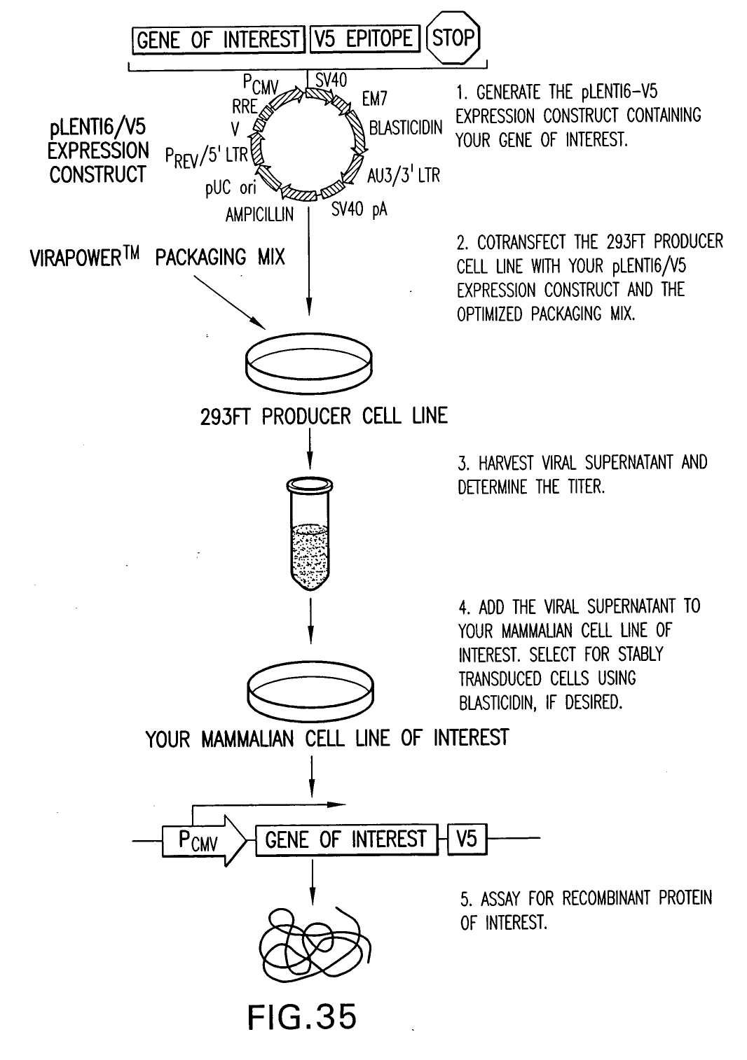

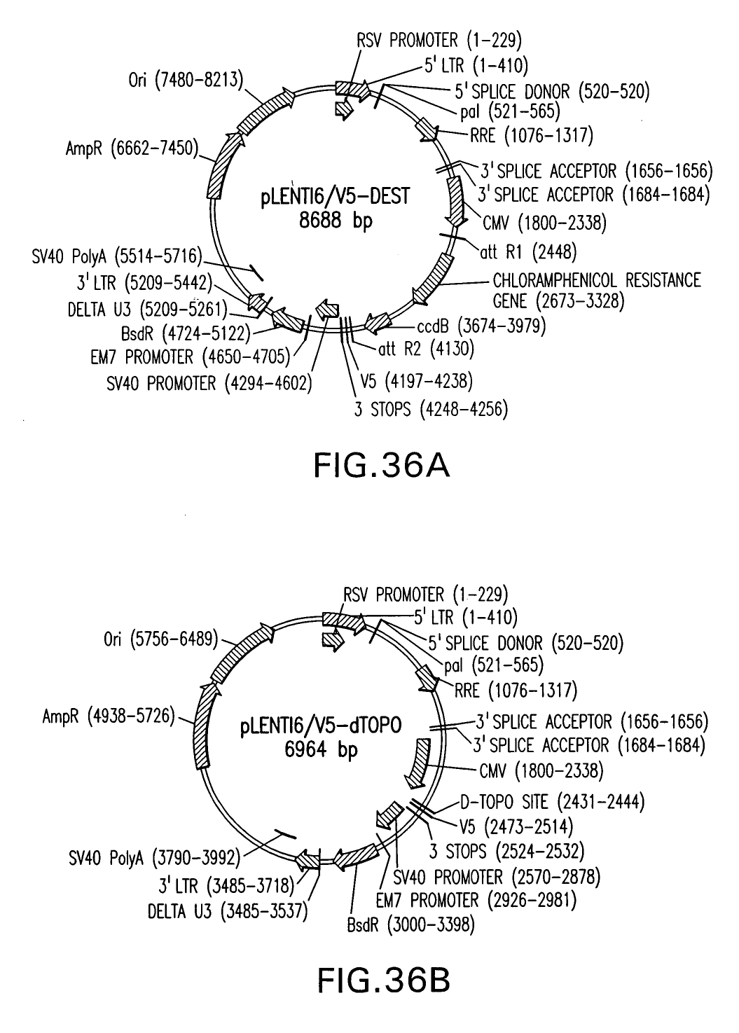

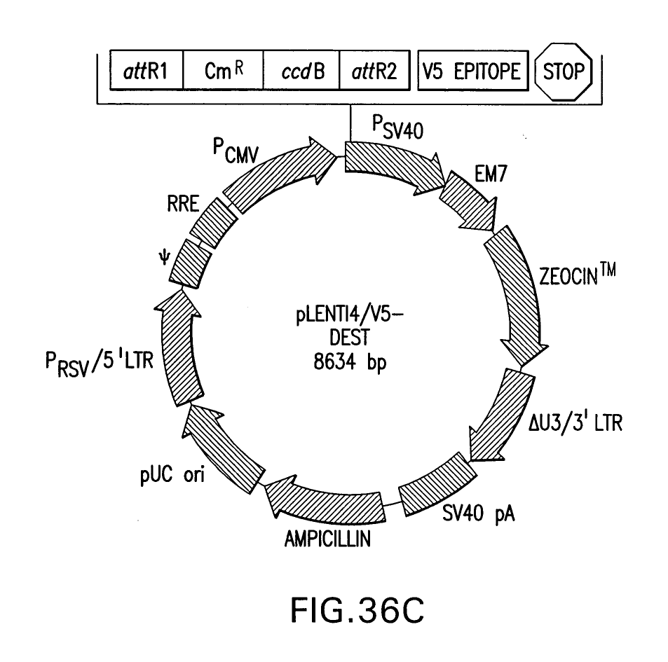

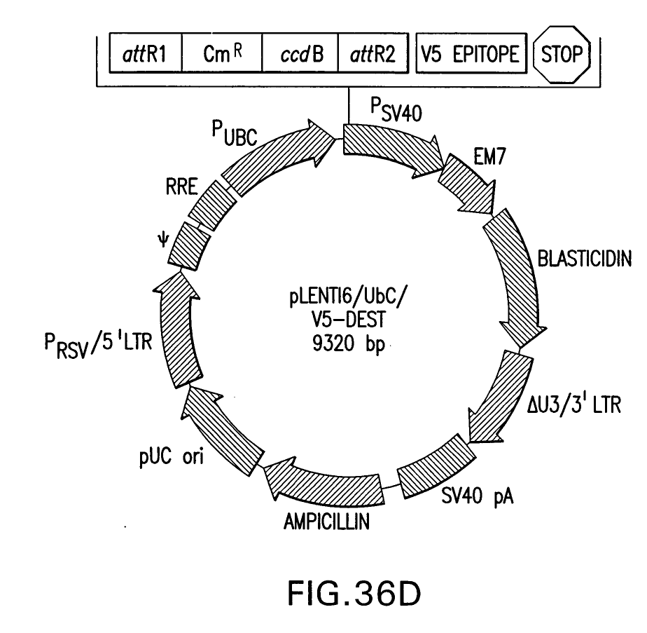

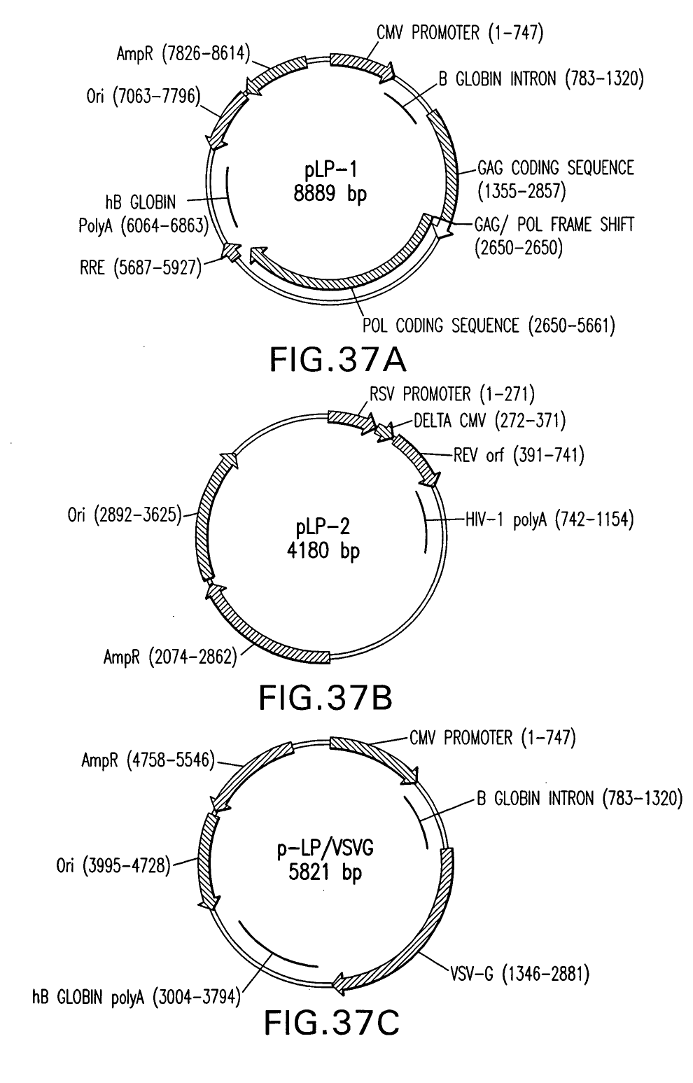

The present invention permits one skilled in the art to create replication-incompetent lentiviruses to deliver and express one or more sequences of interest (e.g., genes). These viruses (based loosely on HIV-1) can effectively transduce dividing and non-dividing mammalian cells (in culture or in vivo), thus broadening the possible applications beyond those of traditional Moloney (MLV)-based retroviral systems (Clontech, Stratagene, etc.). Directional TOPO and GATEWAY™ lentiviral vectors have been created to clone one or more genes of interest with a V5 epitope, if desired. The vectors also carry the blasticidin resistance gene (bsd) to allow for the selection of transduced cells. Without additional modifications, these vectors can theoretically accommodate up to ˜6 kb of foreign gene. Three supercoiled packaging plasmids (gag/pol, rev and VSV-G envelope) are provided to supply helper functions and viral proteins in trans. Finally, an optimized producer cell line (293FT) is provided that will facilitate production of high titer virus. A schematic representation of the production of a nucleic acid molecule comprising all or a portion of a lentiviral genome is shown in FIG. 35. Plasmid maps of vectors adapted for use with GATEWAY™ and topoisomerase cloning in the production of nucleic acid molecules comprising all or a portion of a lentiviral genome are shown in FIGS. 36A (pLenti6/V5-DEST), 36B (pLenti6/V5-D-TOPO®), 36C (pLenti4/V5-DEST), and 36D (pLenti6/UbC/V5-DEST) respectively. The nucleotide sequences of the plasmids are provided in Tables 17-20. Plasmid maps of the three packaging plasmids pLP1, pLP2, and pLP/VSVG are shown in FIGS. 37A, 37B, and 37C respectively and the nucleotide sequences of these plasmids are provided as Tables 21, 22, and 23, respectively.

Retroviruses are RNA viruses that reverse transcribe their genome and integrate the DNA copy into a chromosome of the target cell. It was discovered that the retroviral packaging proteins (gag, pol and env) could be supplied in trans, thus allowing the creation of replication incompetent viral particles capable of stably delivering a gene of interest. These retroviral vectors have been available for gene delivery for many years (Miller et al., (1989) BioTechniques 7:980-990). One significant advantage of retroviral-based delivery is that the gene of interest is stably integrated into the genome of the host cell with very high efficiency. In addition, no viral genes are expressed in these recombinant vectors making them safe to use both in vitro and in vivo. However, one main drawback to the traditional Moloney-based retroviruses is that the target cell must undergo one round of cell division for nuclear import and stable integration to occur. Traditional retroviruses do not have an active mechanism of nuclear import and therefore must wait for the host cell nuclear membrane to breakdown during mitosis before they can access the host genomic DNA (Miller et al., (1990) Mol. Cell. Biol. 10:4239-42).

Unlike traditional retroviruses, HIV (classified as a “lentivirus”) is actively imported into the nuclei of non-dividing cells (Lewis et al., (1994) J. Virol. 68:510-516). HIV still goes through the basic retrovirus lifecycle (RNA genome reverse transcribed in the target cell and integrated into the host genome); however, cis-acting elements facilitate active nuclear import, allowing HIV to stably infect non-dividing cells (for reviews see Buchschacher et al., (2000) Blood 95:2499-2504, Naldini et al., (1999) “The Development of Human Gene Therapy”, Cold Spring Harbor Laboratory Press, pages 47-60). It is important to note that, for both lentivirus and traditional retroviruses, no gene expression occurs until after the viral RNA genome has been reverse transcribed and integrated into the host genome.

Similar to other retrovirus expression systems, the packaging functions of HIV can be supplied in trans, allowing the creation of lentiviral vectors for gene delivery. With all the viral proteins removed, the gene delivery vector becomes safe to use and allows foreign DNA to be efficiently packaged. In addition, it has been shown that lentiviral (or any retroviral) envelope proteins can be substituted for ones with broader tropism. The substitution of envelope is called pseudotyping, and allows creation of lentiviral vectors capable of infecting a wider variety of cells besides just CD4+ cells. Many have found that the G protein from vesicular stomatitis virus (VSV-G) is an excellent pseudotyping envelope protein that imparts a very broad host range for the virus (Yee et al., (1994) Proc. Natl. Acad. Sci. USA 91:9564-9568). The ability of pseudo-typed lentivirus to infect a broad range of non-dividing cells has led to its extensive use in animal gene delivery and gene therapy (Baek et al., (2001) Hum Gene Ther 12:1551-8, Park et al., (2001) Mol Ther 4:164-73, Peng et al., (2001) Gene Ther 8:1456-63).

### Materials and Methods

Vector constructions. Lentiviral vector materials were received from Cell Genesys (Foster City, Calif., see U.S. Pat. Nos. 5,686,279; 5,834,256; 5,858,740; 5,994,136; 6,013,516; 6,051,427; 6,165,782 and 6,218,187, and Dull et al. (1998) J. Virol. 72(11):8463-8471) and modified to incorporate a blasticidin expression cassette and the V5 epitope tag using standard techniques to create pRRL6/V5 also referred to as pLenti6/V5. The nucleotide sequence of pRRL6/V5 is provided in Table 36. To create the GATEWAY™ Destination vector, pLenti6/V5-DEST, the Destination Vector Conversion cassette B (available from Invitrogen Corporation, Carlsbad, Calif. catalog #11828-019) was ligated into pRRL6/V5. This Destination vectorwas propagated in DB3.1 bacteria in the presence of ampicillin (100 μg/ml) and chloramphenicol (15 μg/ml) to maintain integrity. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the destination vector containing attP sites flanking the ccdB and spectinomycin resistance genes can be selected on ampicillin/spectinomycin-containing media. It has recently been found that the use of spectinomycin selection instead of chloramphenicol selection results in an increase in the number of colonies obtained on selection plates, indicating that use of the spectinomycin resistance gene may lead to an increased efficiency of cloning from that observed using cassettes containing the chloramphenicol resistance gene.

To create the control Moloney retroviral vector, prKAT6/V5-DEST, prKAT (Cell Genesys) was digested with BamHI and filled-in with Klenow. This was ligated to the 2732 bp fragment, containing the DEST cassette and SV40-Bsd^{R} cassette, resulting from the digestion of pLenti6/V5-DEST with SpeI and Acc65I followed by Klenow fill-in and gel purification. This Destination vector was propagated in DB3.1 bacteria in the presence of ampicillin (100 μg/ml) and chloramphenicol (15 μg/ml) to maintain integrity. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the destination vector containing attP sites flanking the ccdB and spectinomycin resistance genes can be selected on ampicillin/spectinomycin-containing media. It has recently been found that the use of spectinomycin selection instead of chloramphenicol selection results in an increase in the number of colonies obtained on selection plates, indicating that use of the spectinomycin resistance gene may lead to an increased efficiency of cloning from that observed using cassettes containing the chloramphenicol resistance gene.

To create the expression control vector, pLenti6/V5-GW/lacZ, and the cognate Moloney retroviral control vector, prKAT/V5-GW/lacZ, GATEWAY™ LR reactions were performed with each of the DEST vectors and an entry vector having a copy of the lacZ gene with no stop codon according to the manufacturer's protocol.

Directional TOPO adaptation. The pRRL6/V5 vector was propagated in ampicillin (100 μg/ml) and blasticidin (10 μg/ml) to maintain integrity and reduce backgrounds in the TOPO adaptation. The pRRL6/V5 vector was Directionally TOPO-adapted at the EcoRI (5′ end) and XhoI (3′-end) sites. EcoRI buffer (New England Biolabs, Beverly, Mass.) was used in the digest throughout; vectors were digested first for 3 hours with XhoI at 6 units of enzyme/μg of DNA followed by a 3 hour digestion with EcoRI at 4 units of enzyme/μg DNA. Digested DNA was purified by Phenol/Chloroform/Isoamyl alcohol (PCA) extraction, Ethanol precipitation, 80% Ethanol wash, followed by isopropanol precipitation and another 80% ethanol wash to remove the enzymes and the ˜30 bp multicloning site between the EcoRI and XhoI sites. At this point, the concentration of the cut DNA was quantitated and 10 ng was transformed into chemically competent TOP 10 E. coli to assess the amount of uncut vector (vector that had recombined to delete the multicloning site, or the original vector which “evaded” both restriction enzymes activity).

The oligonucleotides used for directional adaptation are listed below:

EcoRI (5′ end): Non-Regenerative Site

| | |
|---|---|
| Topo-D1 | 5′ P-AATTGATCCCTTCACCGACATAGTACAG 3′ |
| Topo-D2 | 5′ P-GGTGAAGGGATC 3′ |

XhoI (3′ end): Regenerative Site

| | |
|---|---|
| Topo-D6 | 5′ P-TCGAGCCCTTGACATAGTACAG 3′ |
| Topo-D7* | 5′ P-AAGGGC 3′ |

The oligonucleotides were used as pairs: Topo-D1/D2 and Topo-D6/D7 in 200 fold molar excess to vector (51 μg of Topo-D1/D2 pair and 40 μg of Topo-D6/D7 per 100 μg vector DNA). Topo-D1 and D2 were paired in 2.3 to 1 mass ratio, respectively. Topo-D6 and D7 were paired in 3.7 to 1 mass ratio, respectively.

50 units of T4 DNA Ligase (New England Biolabs, Beverly, Mass.) per 1 μg of vector DNA was used in an overnight ligation (˜16 hours) in a 14° C. water bath to ligate the adapter oligonucleotides to the vectors. Subsequently, the sample was heated at 67.5° C. for 15 minutes and then re-digested with EcoRI at 2 units of enzyme/μg vector DNA for 1.5 hours.

Free oligonucleotides were purified away from the oligonucleotide-adapted vector by PCA extraction and a Modified S.N.A.P. column purification protocol, as follows: The PCA extracted DNA (top aqueous phase) was added to 5 volumes of Modified Binding Buffer (MBB) [60% of S.N.A.P. Binding buffer: 40% of (100%) isopropanol], mixed and loaded onto a S.N.A.P. mini or midi (B) column; and the flow through was reloaded back onto the column once more. The column was then washed twice with SNAP Wash buffer, once with the Final Wash buffer (EtOH) and eluted in TE (60-100 μl for mini column and 750 μl for midi column) and concentration determined spectrophotometrically (OD_{260/280}) producing pLenti6/V5-D-TOPO™.

At least 50 μg of the oligo adapted vector was “Charged” with vaccinia topoisomerase in the following reactions (reagents added in the order listed):

Topo Charging with Kinase

| Volume | | Reagent | Final Concentration | |
|---|---|---|---|---|
| # | μl | Topo adapted & purified | | |
| | | DNA (at least 50 μg) | | |
| # | μl | Topo D-70 annealing | 0.2 | μg/μg vector DNA |
| | | oligo | | |
| 50 | μl | Vaccinia Topo Enzyme | 1 | μg Topo/μg vector DNA |
| | | (1 mg/ml) | | |
| # | μl | Water | | |
| 5.3 | μl | 1 M Tris pH 7.5 | 15 | mM |
| 350 | μl | Total | | |

Incubate the reaction at 37 degrees Celsius water bath for 10 minutes and then add:

| Volume | | Reagent | Final Concentration | |
|---|---|---|---|---|
| 16.5 | μl | 100 mMATP | 1.35 | mM ATP |
| | | | (33 | mM ATP/μg DNA) |
| 4 | μl | 1 M _{MgCl2} | 10 | mM MgCl₂ |
| 33 | μl | 10 Units/μl LTI | 6.6 | Units/ |
| | | T4 DNA Kinase | 1 | μg DNA |
| 403.5 | μl | Total | | |

Incubate the reaction at 37 degrees Celsius water bath for 5 minutes and then load all of reaction into Q-column.

TOPO Vector Purification. Q-column purification was performed on the TOPO-charged sample with a 0-1M NaCl (50 mM Tris pH 7.5) gradient as reported for the TOPO-Adapted Entry vectors. DNA fluorescence characterization in the presence of Hoechst dye number 33258 (Sigma catalog #B-2883) was used to quantitate the concentration of individual or pooled fractions containing column purified TOPO-charged vector. In general, approximately 50% of the total DNA loaded onto the column is lost during the purification and the vector-TOPO complexes are eluted in ˜500 mM NaCl. An equal volume of 2× TOPO-Vector Buffer (50 mM Tris 7.5, 2 mM EDTA, 2.5 mM DTT, 0.1 mg/ml BSA, 0.1% Triton X 100, 90% glycerol) is added to the sample fractions. Therefore, the final TOPO Vector Buffer=50 mM Tris 7.5, 1 mM EDTA, 1.25 mM DTT, 0.05 mg/ml BSA, 0.05% TritonX-100, 45% Glycerol. Samples are stored at −20 degrees Celsius until tested.

Standard Topogation reactions were set-up as follows:

1 μl Topo-charged vector

1 μl Directional insert PCR product*

1 μl Salt Solution or 1 μl water

3 μl water *Depending on the concentration of Topo-charged vector, PCR product insert should be adjusted to maximize yield. Ratio of 1 ng vector: 1-2 ng 750 bp insert (Or 1:10-20, vector:insert molar ratio) give good yields.

The topogation reactions were incubated at room temperature for 5 min. Two microliters of the reaction was added to TOP 10 cells, incubated on ice for ˜20 min, heat shocked for 40 seconds at 42° C., placed on ice, and then 250 μl of SOC was added to the transformed cells. Cells were shaken at 37° C. for 1 hr and 100 μl of the cell mixture was plated on LB-amp plates containing blasticidin (50 μg/ml final).

Cell culture and growth arrest. 293FT producer cells (available from Invitrogen Corporation, Carlsbad, Calif., catalog number R7007) were cultured in DMEM/10% FBS/L-glutamine/non-essential amino acids/penicillin/streptomycin containing 500 μg/ml G418. MJ90 primary human foreskin fibroblasts, HT1080 human fibrosarcoma (ATCC #CCL-121) and HeLa cervical carcinoma cells (ATCC #CCL-2) were cultured in DMEM/10% FBS/non-essential amino acids/penicillin/streptomycin. Chinese hamster ovary cells (CHO-K1, ATCC #CCL-61) were cultured in Hams F12/10% FBS/L-glutamine/penicillin/streptomycin. For blasticidin selections, the following final concentrations were used: HT1080: 10 μg/ml, CHO: 5 μg/ml, HeLa: 2 μg/ml.

MJ90 primary cells were growth arrested by contact inhibition. Briefly, 1×10⁵ cells were plated per well of a 6-well plate and media changes were performed every 3 days for 7 to 14 days, or until a quiescent monolayer was achieved. Aphidicolin (Sigma, St. Louis, Mo., catalog number #A0781) was used to arrest HT1080 cells at the G1/S transition. Exponentially growing cultures were plated at 2×10⁵ cells per well of a 6-well plate and the following day fresh media was supplied containing 1 μg/ml aphidicolin. Transductions of aphidicolin-arrested cells were performed in the continued presence of drug.

Primary, post-mitotic rat hippocampal and cortical neuronal tissues were received from BrainBits Inc. (Dr. Greg Brewer, University of Southern Illinois). Tissues were dissociated with a Pasteur pipette, spun down at 1100 rpm for one minute and resuspended in NeuroBasal Medium (Invitrogen Corporation, Carlsbad, Calif., Gibco #21103-049) containing B27 supplement (Invitrogen Corporation, Carlsbad, Calif., Gibco #17504-010), 0.5 mM L-glutamine and 25 μM glutamate. 5×10⁴ hippocampal or 1×10⁵ cortical neurons were plated per well in 24-well plates. Four days after plating, half of the medium was removed and replaced with complete NeuroBasal Medium (as above) but without the glutamate. The following day, cells were transduced with virus.

Virus production. For optimal virus production, 5×10⁶ 293FT cells were plated per 100 mm plate. Twenty-four hours later, the culture medium was replaced with 5 ml OptiMem/10% FBS (Opti-MEM®, catalog no. 22600050, Invitrogen Corporation, Carlsbad, Calif.) and cells were quadruple co-transfected, as follows. 12 μg DNA total, at a mass ratio of 1:1:1:1 pLenti6/V5/gene:pLP-1:pLP-2:pLP/VSVG (3 μg of each DNA) was mixed with 1.5 ml of OptiMem media. In a separate tube, 36 μl of Lipofectamine 2000 was also mixed with 1.5 ml of OptiMem media. After a 5-minute incubation period at room temperature, the two mixtures were combined and incubated at room temperature for an additional 20 minutes. At the completion of the incubation period, the transfection mixture was added to the cells dropwise and the culture plate was gently swirled to mix. The following day the transfection complex was replaced with complete media (DMEM, 10% FBS, 1% penicillin/streptomycin, L-glutamine and non-essential amino acids). Forty-eight to seventy-two hours post transfection, the virus-containing supernatants were harvested, centrifuged at 3000 rpm for 15 minutes to remove dead cells and placed in cryovials in 1 ml aliquots. Titers were performed on fresh supernatants (see below) and the remaining viral aliquots were stored at −80° C.

Viral titering and transduction. All applications of virus to cells were performed in the presence of 6 μg/ml polybrene (Sigma, St. Louis, Mo., catalog #H9268) and media changes were performed 12-24 hours post transduction. For titering virus, 6-well plates were seeded at 2×10⁵ cells per well with HT1080 cells the day before transduction. One well served as an untransduced control (mock) and the remaining five wells contained 1 ml each of ten-fold serial dilutions of viral supernatant ranging from 10⁻² to 10⁻⁶ (see example below). The dilutions were mixed by gentle inversion (dilutions should not be vortexed) prior to adding to cells. 6 μg/ml of polybrene was added to each well. The plate was gently swirled to mix. The following day, the media was replaced with complete media. Forty-eight hours post transduction, the cells were placed under 10 μg/ml blasticidin selection (Invitrogen). After 10 to 12 days of selection, the resulting colonies were stained with crystal violet: A 1% crystal violet solution was prepared in 10% ethanol. Each well was washed with 2 ml PBS followed by 1 ml of crystal violet solution for 10 minutes at room temperature. Excess stain was removed by two 2 ml PBS washes and colonies visible to the naked eye were counted to determine the viral titer of the original supernatants. In a typical example, colonies can be counted in the 10⁻⁵ and 10⁻⁶ dilutions.

Protein analysis. Total cell lysates were prepared using NP-40 lysis buffer (Igepal CA636, Sigma, St. Louis, Mo.) and the proteins (20 μg/lane) were separated on a 4-20% Novex Tris-Glycine gel. Following electrophoresis, the proteins were transferred to nitrocellulose. Western blotting was performed using the Western Breeze Chemiluminescence Kit (Invitrogen Corporation, Carlsbad, Calif.), using anti-large T antigen mouse monoclonal antibody (e.g., catalog no. 554149, BD Biosciences Pharmingen, San Diego, Calif.), anti-lacZ rabbit polyclonal antibody (1:5000 dilution, Invitrogen Corporation, Carlsbad, Calif.) or anti-V5 mouse monoclonal antibody (1:2000 dilution, Invitrogen Corporation, Carlsbad, Calif.). Beta-galactosidase activity assays were performed using the Galacto-Light Plus Kit (Tropix, Applied Biosystems, Foster City, Calif.) according to the manufacturer's instructions. Beta-galactosidase staining was performed using the β-Gal Staining Kit (Invitrogen Corporation, Carlsbad, Calif.) according to the manufacturer's instructions.

The present invention provides a production and expression kit that allows easy construction, production and use of nucleic acid molecules comprising all or a portion of a lentiviral genome (e.g., lentiviral vectors). Aspects of the invention include, but are not limited to, 1) directional TOPO® and GATEWAY™ Destination pLenti6/V5 vectors with a useful selectable marker and epitope tag, 2) optimized virus production conditions and cell lines to reproducibly achieve >10⁵ infectious viral particles per ml, 3) stable gene delivery and expression of at least two genes into actively dividing mammalian cells, and 4) transduction of at least two non-dividing cell types.

A four plasmid co-transfection is used to create infectious lentiviral vectors (Dull, et al., (1998) J. Virol. 72:8463-8471). One of the vectors (pLenti6/V5-DEST, pLenti6/V5-D-TOPO®, pLenti4/V5-DEST, or pLenti6/UbC/V5-DEST) contains the gene of interest and is packaged into the virions (for vector maps, see FIGS. 36A-D). The other three plasmids are co-transfected to supply the viral proteins in trans. None of these three vectors are packaged into the virions. Each vector and a description of its features is described in more detail below. Vector maps are provided as FIGS. 37A, 37B, and 37C.

pLenti6/V5-DEST or pLenti6/V5-D-TOPO carries gene of interest and blasticidin resistance gene and is packaged into viral particles. The vector contains the RSV promoter, which enhances production of the viral genomic RNA in the producer cell and removes dependence on HIV tat protein. The vector also contains viral 5′ and 3′ LTRs (Long Terminal Repeats), which are required for viral packaging and reverse transcription of the viral RNA. The 3′ LTR also contains polyA signal. The vector contains the Ψ (psi) packaging signal. Nuclear export of unspliced viral genomic RNA in the presence of rev occurs as a result of the RRE (Rev-Responsive Element) present in the vector. The vector also incorporates 5′ and 3′ splice sites that result in the removal of psi and RRE making expression of the gene of interest no longer rev-dependent in the host cell. The vector also contains Delta U3, a 400 bp deletion in the 3′ LTR that gets copied to the 5′ LTR after reverse transcription of the viral genome in the transduced target cell. This results in “self-inactivation” of the 5′ LTR for biosafety.

pLP1 expresses HIV-1 gag and pol genes in trans and is not packaged into viral particles produced with this system. The plasmid contains the RRE, which makes expression of gag/pol genes rev-dependent (for safety purposes).

pLP2 expresses HIV-1 rev gene in trans and, like pLP1, is not packaged into viral particles. The plasmid encodes the rev protein, which is required for gag/pol expression and for nuclear export of the unspliced viral genome (from pLenti6/v5-DEST or D-TOPO®) for packaging into the virions.

pLP/VSVG expresses the VSV-G envelope gene in trans. The plasmid is not packaged into viral particles, however, the VSV-G protein is incorporated into the viral particle. VSV-G is a non-HIV envelope that broadens the host range and stabilizes the viral particles (Yee 1994).

### Results and Discussion

Vector construction. The vector pRRLsin.hCMV.GFPpre was used as the starting material from Cell Genesys. This vector contains the essential elements for lentiviral packaging (e.g., 5′ and 3′ LTRs, psi packaging signal, rev responsive element (RRE) and necessary splice sites; see above for descriptions). In addition, it contains a deletion in the 3′ LTR (called “delta U3”) that results in a self-inactivation of the 5′ LTR after integration of the viral genome into the genome of the target cell (Dull 1998, Zufferey et al., (1998) J. Virol. 72:9873-80). This is an additional safety measure (see “Safety” section below) and has no effect on vector performance since the 5′ LTR is only needed during viral production, not gene expression in the target cell (Zufferey 1998). Finally, all polyadenylation (polyA) functions are supplied by the 3′ LTR. The 3′ LTR serves as the polyA for the viral genome (driven by the RSV/5′ LTR), the CMV promoter (gene of interest) and the SV40 promoter (blasticidin resistance). No heterologous polyA signals should ever be included between the LTRs or viral production will be severely compromised due to transcription termination prior to the 3′ LTR. The downstream SV40 polyA in the pLenti6/V5 vectors simply enhances viral genomic RNA production in the producer cells and is not packaged into the virions.

TOPO adaptation and purification. Fifty micrograms of TOPO-charged pLenti6/V5-D-TOPO® was loaded on the Q-column and fractions containing the purified vector were collected in seven 0.5 ml fractions. The peak fraction (fraction 41) contained ˜20 μg of DNA by Hoechst (H 33258) dye DNA fluorescence characterization and was eluted of at ˜500 mM NaCl. Only this fraction was analyzed, however fractions 39-45 also contained TOPO-charged DNA. The fractions were diluted in 2× TOPO dilution buffer, so fraction 41 contained vector at ˜20 ng/μl final concentration. TOPO transformation results, using fraction 41 in two experiments (one with 750 bp insert, one with lacZ-alpha), are shown in Table 24.

TABLE 24
pLenti6/V5-D-TOPO ® transformations.

| | | #colonies/ | | | | | |
|---|---|---|---|---|---|---|---|
| Vector | Insert | μl vector | | Orientation (% correct) | | % background | |
| pLenti6/ | None | 162 | 612 | — | — | — | — |
| V5-D-TOPO | | | | | | | |
| | 750 bp test | 1665 | — | 9/10 (90%) | — | 9.7% | — |
| | LacZ alpha | — | 4464 | — | 17/18 (94%) | — | 13.7% |

Note: Transformation efficiency with pUC19: 4008 colonies/10 pg = 4.0 × 10⁸ cfu/μg efficiency

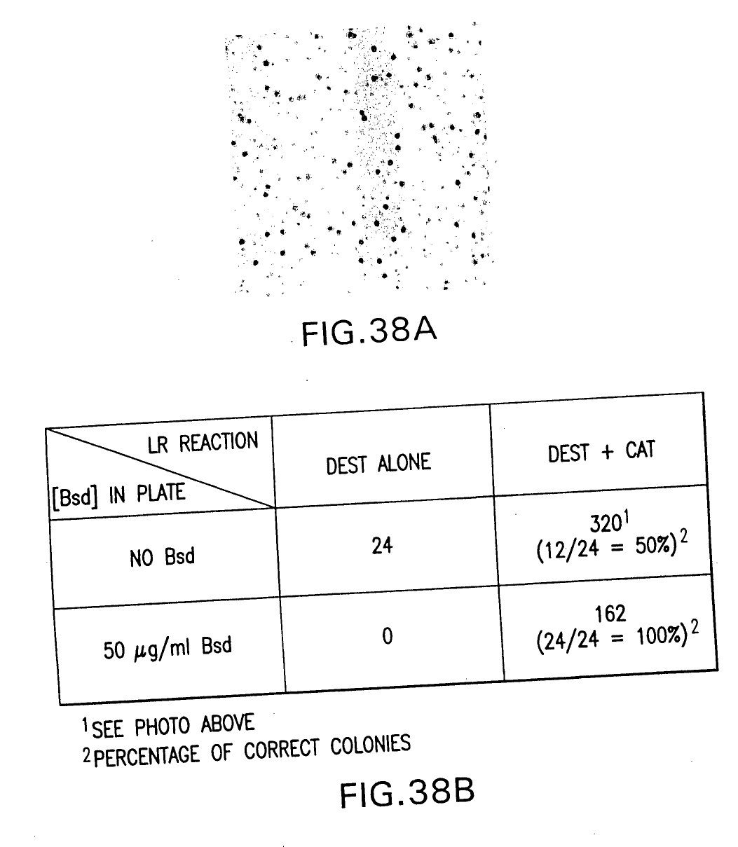

Vector instability. While performing manipulations on the vectors, it was discovered that the presence of 182 basepairs of direct repeat present in the LTRs was triggering homologous recombination when transformed into TOP10 and plated on LB-amp. This resulted in a visible colony phenotype. In clones where LTR recombination occurred the colonies were large, while unrecombined (correct) clones resulted in small colonies. FIG. 38 shows the results of an experiment in which two LR reactions were performed with either pLenti6/V5-DEST alone or pLenti/V5-DEST plus pENTR/CAT and 3 μl of each was transformed into TOP10 cells. 100 μl of the transformations were plated on regular LB-amp plates (No Bsd in FIG. 38) or LB-amp containing 50 μg/ml blasticidin. After overnight incubation at 37 degrees, colonies were photographed (FIG. 38A) and counted (FIG. 38B). Twenty-four clones (twelve each from two independent experiments) from the DEST+CAT plates (+/−Bsd) were randomly picked and screened by restriction digest to determine the percentage of correct clones.

Since blasticidin resistance (driven by the EM7 promoter) is present between the LTRs, it was found that spreading blasticidin on the bacterial plate (to a final concentration of 50 μg/ml) resulted in all small colonies, none of which contained the LTR recombination product (not shown). This was further confirmed when GATEWAY™ LR reactions were performed using pLenti6/V5-DEST with and without including the pENTR-CAT plasmid (FIG. 38B). Without blasticidin in the plate, background colonies arose from the DEST vector alone and only 50% of the DEST+CAT clones were intact. However, when blasticidin was included in the plate, the DEST vector alone gave no background colonies and all DEST+CAT clones were correct and intact (FIG. 38B). Therefore, it is recommended that one of two approaches be use when introducing a gene of interest into pLenti6/V5 vectors: 1) if high efficiency cloning (i.e. library-scale) is not required, simply pick only the small colonies for miniprep analysis; or 2) to ensure ˜100% correct clones, include blasticidin (50 μg/ml) in the bacterial plate following transformation. It has been observed that once a clone is isolated and shown to be intact, it appears to remain stable over multiple rounds of large-scale propagation without blasticidin. Nevertheless, it is recommended that each DNA preparation be verified by restriction digest prior to proceeding to virus production.

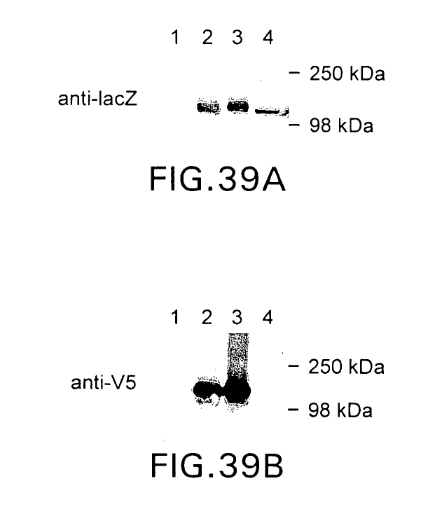

Transient transfection expression testing. To verify protein expression and the functionality of the V5 epitope tag, the lacZ ORF (with or without a stop codon) was GATEWAY™ cloned into pLenti6/V5-DEST. The resulting attB expression clones were transiently transfected into COS cells and analyzed by anti-β-galactosidase and anti-V5 western blotting (FIG. 39). COS-7 cells were transiently transfected with:

Lane 1: mock; Lane 2: pcDNA3.1/V5His/lacZ; Lane 3: pLenti6/V5-GW-lacZ (no stop); Lane 4: pLenti6/V5-GW-lacZ (with stop); and lysates were analyzed by anti-lacZ or anti-V5 western blotting as indicated.

Compared to pcDNA3.1/V5His/lacZ, pLenti6/V5-GW/lacZ expressed equally well with and without the V5 epitope tag. In addition, lacZ (no stop) resulted in an efficiently expressed V5-tagged fusion protein (lane 3). This vector can be used as an expression control vector and may be included in kits of the invention.

Virus production optimization. Previous reports had indicated that virus production is maximal in human 293 cells that express the SV40 large T antigen (Naldini, et al., (1996) Proc. Natl. Acad. Sci. USA 93:11382-11388). Virus production was tested in several neomycin-resistant 293FT clones.

These cell lines were created by stably transfecting 293F cells with the pCMV/Sport6-T antigen plasmid in which the SV40 origin had been deleted. 293FT clone #42 was found to produce the highest levels of infectious virus. The expression of the SV40 large T antigen was confirmed by western blot analysis and producer cell stocks were propagated in G418 to maintain the large T antigen expression.

Since the production of virus requires a quadruple transfection, the importance of the ratio of the four plasmids was tested. Published reports suggested a variety of ratios as “optimal” (Dull 1998; Naldini 1996; Mochizuki et al., (1998) J. Virol. 72:8873-8883), so each published ratio was evaluated and compared to the simple 1:1:1:1. Little difference was seen between the simple 1:1:1:1 and the more elaborate ratios (e.g. 4:2.6:1:1.4). The highest and most reproducible titers were generated using a simple ratio of 1:1:1:1. The most effective time course for production of virus was determined. Various genes were cloned into pLenti6/V5 and virus was produced in 293FT cells according to the following optimized protocol

Day 0 Plate 5×10⁶ 293FT per 100 mm plate

Day 1 Four plasmid co-transfection (ratio=1:1:1:1)

12 μg DNA total (3 μg each)

36 μl Lipofectamine 2000

Day 2 Replace media

Day 3-4 Harvest supernatant containing virus

Spin 3000 rpm×15′ and/or filter 0.45 μm

Aliquot supernatant, use for titering and store −80° C.

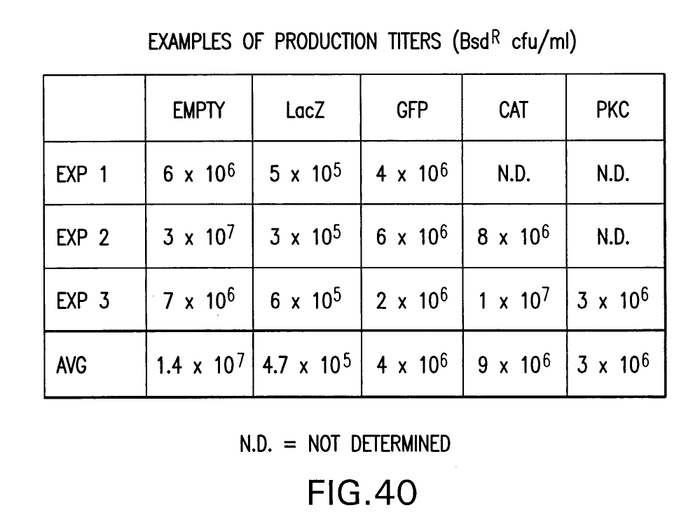

Independent virus productions, of either the empty vector (pLenti6/V5-DEST) or carrying lacZ, GFP, CAT or protein kinase C, were titered on HT1080 cells by counting the number of resulting blasticidin-resistant colonies generated per ml of supernatant and the results are shown in FIG. 40. The optimized protocol which included high density plating of the 293FT cells (5×10⁶ cells per 100 mm plate) and the optimal lipid to DNA ratio using Lipofectamine 2000. It was found that viral supernatants can be harvested either 2 or 3 days post transfection with minimal differences in viral yield. Presumably, the short half-life of the virus in culture media at 37° C. negates any advantage of viral accumulation over one extra day. For storage, aliquotting viral stocks at −80° C. is recommended. Anywhere from 0 to 10% loss of viral titer for each freeze/thaw cycle of crude supernatant was observed.

The size of the inserted gene of interest can affect the viral titer. Three different genes were GATEWAY™ cloned into pLenti6/V5-DEST (lacZ, CAT and protein kinase C) and one gene was directionally TOPO cloned (GFP). Viral production was compared between these four gene-containing vectors and an empty vector, pLenti6/V5 (FIG. 40). Averages from three independent experiments showed that the empty vector yielded the highest viral titer (average 1.4×10⁷ cfu/ml), while the largest insert (lacZ) yielded the lowest titers (average 4.7×10⁵ pfu/ml). Inserted genes of intermediate size (GFP, CAT and PKC) yielded titers somewhere in between (4×10⁶, 9×10⁶ and 3×10⁶; respectively). These data indicated that both the GATEWAY™ and TOPO versions of these vectors can produce viral supernatants that easily exceed a viral titer of 10⁵, even with the large lacZ gene. The wild type HIV-1 genome is approximately 10 kb and the elements present in pLenti6/V5 vectors add up to 3.7 kb. Therefore, the theoretical gene-packaging limit is approximately 6 kb.

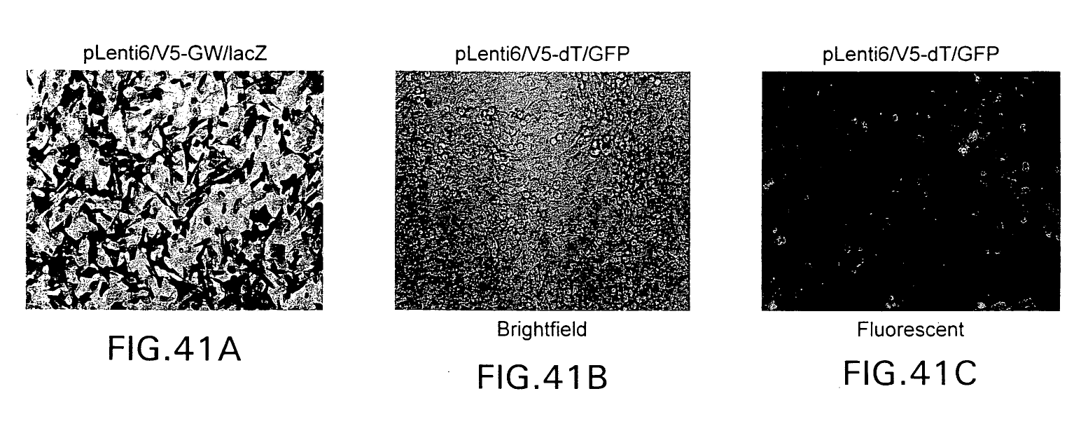

Viral gene delivery and expression. The ability of the lentiviral vectors to deliver and express a variety of genes was further investigated. HT1080 cells were transduced with either Lenti6/V5-GW/lacZ virus (GATEWAY™) or Lenti6/V5-dT/GFP virus (D-TOPO®) and selected for 10 days with 10 μg/ml blasticidin. LacZ was visualized using the β-Gal Staining kit and GFP was visualized using the fluorescent microscope (FIG. 41). Both the GATEWAY™ lacZ and the D-TOPO® GFP vectors efficiently generated heterogeneous pools of stably transduced cells in which nearly 100% of the cells expressed the heterologous gene. In addition to HT1080, HeLa and CHO cells have been stably transduced with similar efficiencies and levels of gene expression.

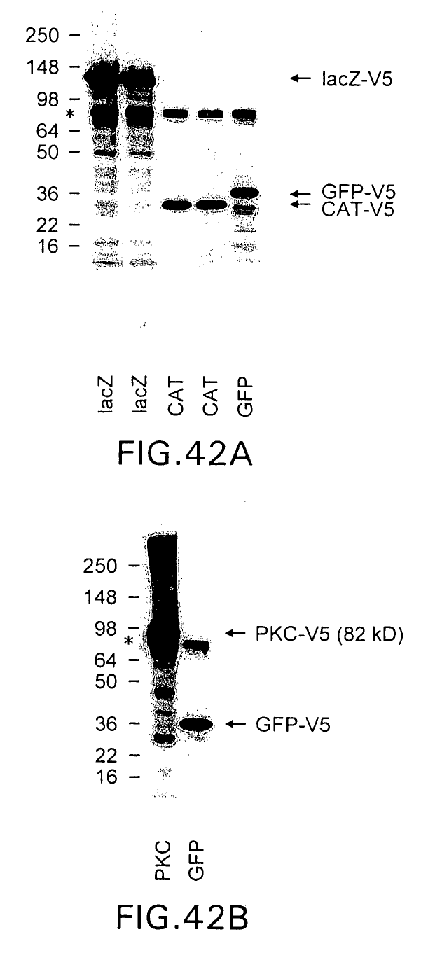

To confirm the above results, and to verify that a functional V5 epitope tag was efficiently added to the expressed proteins, cell lysates were prepared from HT1080 cells stably transduced with either the lacZ, CAT, GFP or protein kinase C viruses (FIG. 42). HT1080 cells were transduced in duplicate with lentiviral vectors carrying genes for either lacZ, CAT, GFP or PKC and selected for 10 days with 10 μg/ml blasticidin. Cell lysates were analyzed by anti-V5 western blotting. Molecular weight markers and each V5 fusion protein are indicated. *indicates background V5 band. All four proteins are efficiently expressed and all are properly fused to a detectable V5 epitope. In addition, the delivery and efficient expression of protein kinase C (a “relevant” gene, i.e., not lacZ, GFP or CAT) indicates the robustness and broad applicability of this virus production system.

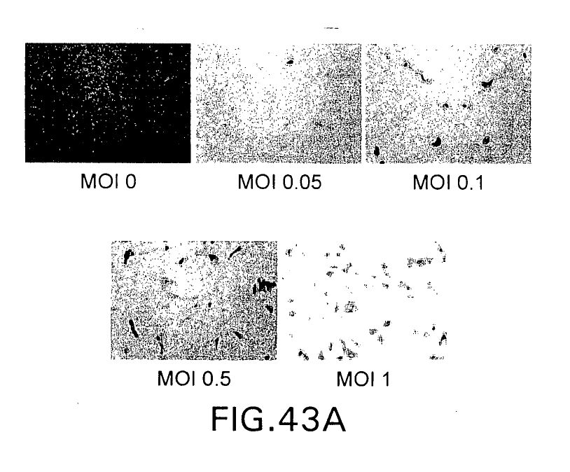

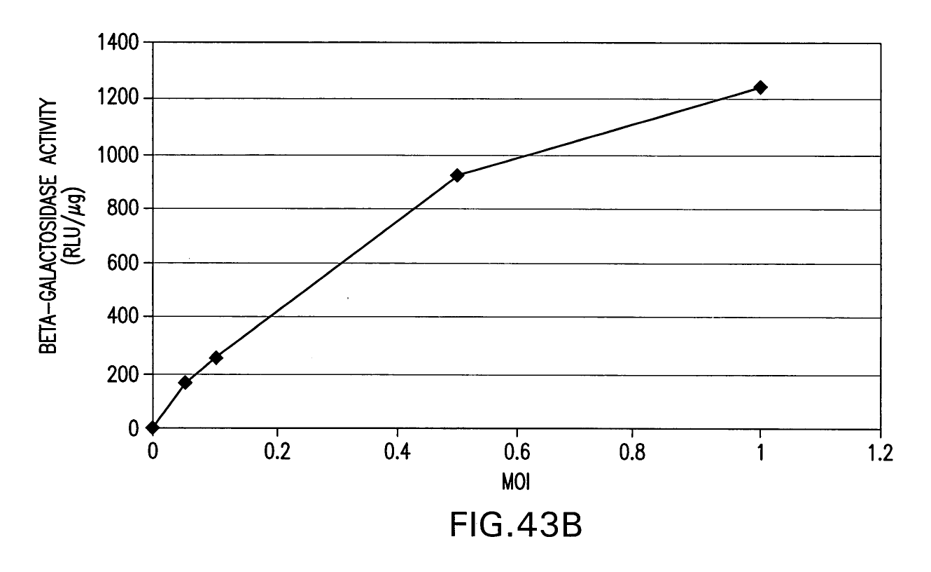

Gene expression is correlated to MOI. Theoretically, the multiplicity of infection (MOI=number of virus per cell) should correlate with gene delivery and expression. To investigate this, HT1080 cells were transduced in duplicate at various MOIs, ranging from 0.05 to 1 (FIG. 43). HT1080 cells were transduced in duplicate with Lenti6/V5-GW/lacZ virus at multiplicities of infection (MOI) of 0.05, 0.1, 0.5 and 1. Forty-eight hours later, cells were either stained for β-Gal (FIG. 43A) or harvested and analyzed for β-Gal activity (FIG. 43B). As the β-Gal staining indicates, an increasing number of cells become lacZ-positive as the MOI increases. At an MOI of 1, greater than 80% of the cells express lacZ. At higher MOIs (e.g. MOI 5), 100% of the cells were transduced. When cell lysates were analyzed for lacZ activity, a near-linear dose response was observed as the MOI increased from 0.05 to 1 (FIG. 43B). At higher MOIs (e.g. MOI 5), the lacZ activity continues to increase, but graph tends to flatten out.

Lentiviral transduction of non-dividing cells. One of the key advantages of lentiviruses over traditional retroviruses is that they are capable of stably transducing non-dividing cells. This significantly expands the potential tranducible target cells to include: 1) growth- or drug-arrested cells in culture, 2) non-dividing primary cell cultures, and 3) animals/tissues. To verify that lentiviral vectors of the invention could perform under these conditions, they were using three different approaches.

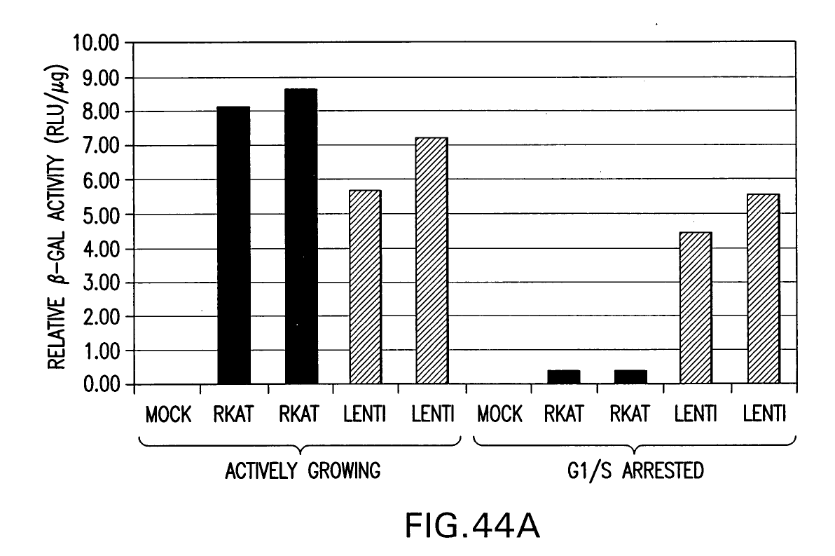

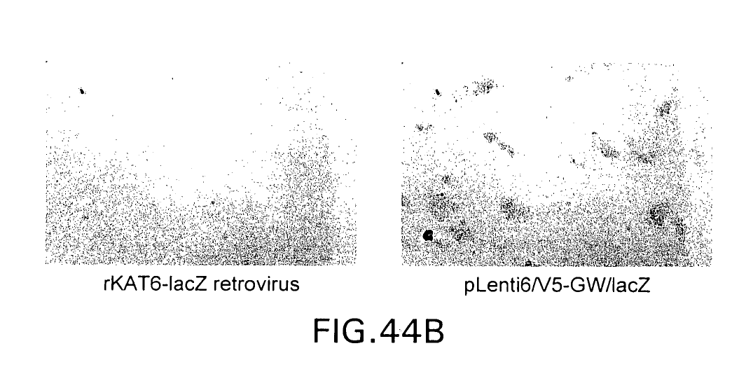

Drug-arrested cells. Actively-growing cells in culture can be arrested at specific phases of the cell cycle using a variety of drugs. This approach is widely used in cell cycle analysis and tumor biology. One commonly used drug, aphidicolin, reversibly binds to DNA polymerase delta and is used to arrest cells at the G1/S transition (Seki et al., (1980) Biochem Biophys Acta 610:413). To test the activity of lentiviral vectors of the invention under conditions of cell cycle arrest, aphidicolin-blocked HT1080 cells were transduced with Lenti6/V5-GW/lacZ virus (FIG. 44A). HT1080 cells were either actively growing or growth arrested at G1/S by aphidicolin and transduced at an MOI of 1, in duplicate, with either rKAT6/V6-GW/lacZ retrovirus or Lenti6/V5-GW/lacZ lentivirus. Forty-eight hours post transduction, cell lysates were analyzed for beta-galactosidase activity. The control virus, rKAT6/V5-GW/lacZ virus, is a traditional Moloney-based retrovirus carrying the same lacZ gene. Both retrovirus and lentivirus were capable of transducing actively growing cells, but only the lentiviral vector was capable of transducing the non-dividing culture.

Quiescent primary cells. The second approach was to apply the lentiviral vectors to non-dividing primary human cultures. A low-passage primary human foreskin fibroblast culture (MJ90, Grand Island) was plated into 6-well format and allowed to grow to confluence. Primary fibroblasts are strongly contact inhibited and can be maintained for many weeks arrested in quiescence (G₀) when maintained as a confluent culture. Contact-inhibited non-dividing quiescent primary human foreskin fibroblasts were transduced with retrovirus (rKAT6/V5-GW/lacZ) and lentivirus (Lenti6/V5-GW/lacZ) at an MOI of 1 and β-Gal stained forty-eight hours post transduction. Similar to the results in aphidicolin-arrested cells, only the lentiviral vector (and not the retroviral rKAT vector) was capable of transducing non-dividing cells. Approximately 50% of the quiescent primary cells were transduced with an MOI of 1 (FIG. 44B).

Post-mitotic primary neurons. Neuronal research is one area where lentiviral vectors can offer significant advantages over other gene transfer methods. Neuronal cultures are typically non-dividing, “post-mitotic” cells that transfect poorly. Traditional Moloney retroviruses are not useful since the cells never go through mitosis. Lentiviral vectors are one solution to overcome these hurdles, and vectors of the invention were tested to determine if they could stably transduce these cells. Primary, post-mitotic rat neuronal tissues (cortical and hippocampal) were received from BrainBits, Inc. and then processed and plated. Four days after plating, cells were transduced at an MOI of 1 with either Lenti6/V5-GW/lacZ lentivirus or rKAT6/V5-GW/lacZ retrovirus. Three days post-transduction, cultures were stained for β-galactosidase. All wells transduced with the lentiviral vectors stained blue, with approximately 50% of the cells expressing detectable β-galactosidase. Conversely, wells transduced with the rKAT retrovirus did not show any β-galactosidase expression. These results indicated that lentiviruses of the invention effectively transduced post-mitotic neurons of either cortical or hippocampal origin.

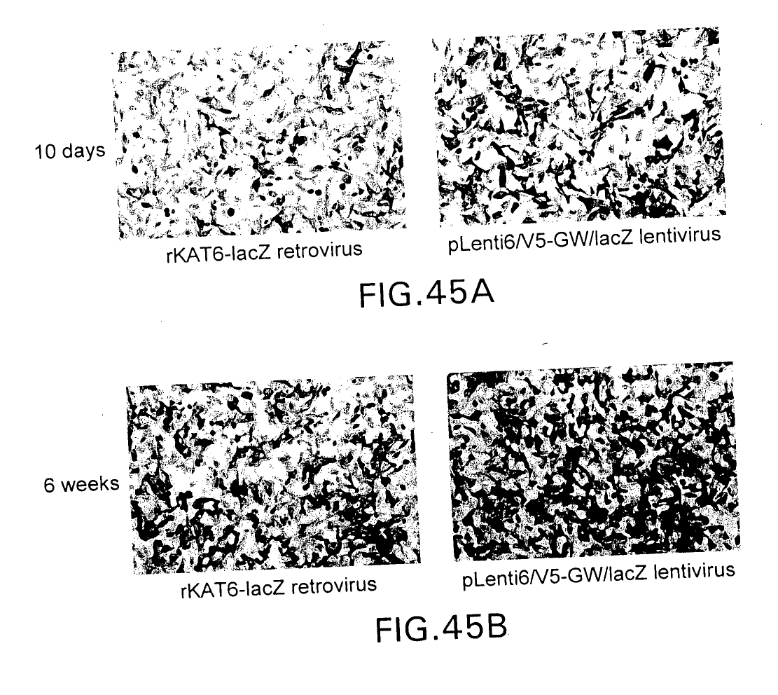

Long-term gene expression from lentiviral vectors. The stability of gene expression after delivery by lentiviral transduction was tested. HT1080 cells were transduced with either the Lenti6/V5-GW/lacZ lentivirus or the rKAT6/V5-GW/lacZ retrovirus and stably selected with 10 μg/ml blasticidin. Cultures were maintained in blasticidin and were β-Gal stained at 10 days (FIG. 45A) and 6 weeks (FIG. 45B) post transduction. No loss of gene expression was observed over 6 weeks in culture, indicating that lentiviral gene delivery is stable and gene expression is persistent even at 6 weeks post transduction.

The present invention describes the generation of infectious lentiviral particles based on the genome and lifecycle of HIV-1. Considerable effort has been put into developing a system that is safe to use and is as far-removed from wild type HIV as possible. Key safety features built into this “3^{rd} generation” system are as follows:

The viral particles produced in this system are replication incompetent and only carry the gene(s) of interest. No other viral species are produced. This also means that none of the structural HIV genes (necessary for production of viral progeny) are present in the packaged viral genome. Only sequences flanked by the viral LTRs will be packaged into virions (i.e., pLenti6/V5 vector). None of the three packaging plasmids contain LTRs (FIGS. 37A-C); so while they are expressed in the producer cell, they are never packaged into the virions. Once a cell is infected (the proper term for this event is “transduced”), the only genes that are delivered and expressed are the gene of interest and the selectable marker. Gag, pol, rev and envelope genes are not present in the viral genome and are therefore never expressed in the target cell, so no new virus can be produced.

The system described above is a four-plasmid system. The necessary HIV-1 genes (gag-pol and rev) have been separated onto individual plasmids, and the non-HIV envelope is on a third plasmid (FIGS. 37A-C). All four plasmids have been engineered not to contain any regions of homology with each other to prevent unwanted recombination events that could lead to the generation of a replication competent virus (Dull 1998). In other words, multiple non-homologous recombination events would need to occur to get all necessary components into one viral genome. In addition, the expression of gag and pol (from pLP1) is rev-dependent, by virtue of the RRE in the gag/pol transcript. This prevents unwanted gag/pol expression if rev is not present (Dull 1998). In other embodiments, one or more of the genes necessary for generation of a replication-incompetent retrovirus according to the methods of the invention (i.e., gag, pol, rev, and a pseudotyping envelope protein) may be expressed from the genome of a host cell. Thus, some or all of the necessary genes may be expressed from plasmids and some or all of the necessary genes may be expressed from the host cell genome. In a particular embodiment, one or more of the necessary genes may be expressed from the host cell genome and at least one gene expressed from the host cell genome may be operably linked to an inducible promoter. In another embodiment, all the genes necessary may be expressed from the genome of a host cell and one or more may be operably linked to an inducible promoter. When more than one gene is operably linked to an inducible promoter, the inducible promoters may be the same or different.

The gene transfer vector pLenti6/V5 has been modified to be “self-inactivating” (Yu et al., (1986) Proc. Natl. Acad. Sci. USA 83:3194-3198, Yee et al., (1987) Proc. Natl. Acad. Sci. USA 84:5197-5201, Zufferey 1998). A deletion has been made in the 3′ LTR (called “delta U3”) that has no effect on the generation of viral genome for packaging in the producer cell. However once the produced virus transduces a target cell, the mechanisms of reverse transcription use the 3′ LTR as a template to create the 5′ LTR. The end result is an integrated viral genome that is defective in both its 5′ and 3′ LTRs, and is no longer capable of producing packagable viral genome. This means that transduction with lentiviral vectors of the invention does not generate a productive infection, instead ending with a gene of interest integrated into the host cell genome.

Despite all of these safety features, the lentivirus produced with this system can still pose a biohazardous risk. As shown above, they are fully capable of transducing primary human cells, thus these viruses should be treated as Biosafety Level 2 organisms. Extra care should be taken when creating viruses carrying harmful or toxic genes (such as activated oncogenes). For further information on BL-2 guidelines and lentivirus handling, please refer to: “Biosafety in Microbiological and Biomedical Laboratories”, 4^{th} Ed. Centers for Disease Control and contact the CDC.

Conclusions. The lentivirus production and expression system of the invention is based on the 3^{rd} Generation lentiviral system created at Cell Genesys (Dull 1998). This system allows one skilled in the art to rapidly clone their gene of interest into a packagable lentiviral vector, via GATEWAY™ or directional TOPO, and provides materials necessary for the creation of infectious viral particles. Finally, these viruses are capable of stably delivering a variety of genes to both actively dividing and non-dividing primary and immortalized human cell lines.

### Example 10

Materials and methods of the present invention (e.g., the ViraPower™ Lentiviral Expression System) allow creation of a replication-incompetent retroviruses (e.g., an HIV-1-based lentivirus), which can then be used to deliver and express a sequence of interest in either dividing or non-dividing eukaryotic (e.g., mammalian) cells. In some embodiments, materials of the present invention may include, but are not limited to, expression plasmids, for example, an expression plasmid that contains the sequence of interest under the control of a suitable promoter (e.g., the human cytomegalovirus (CMV) immediate-early enhancer/promoter; see Andersson, et al. (1989) J. Biol. Chem. 264, 8222-8229; Boshart, et al. (1985) Cell 41, 521-530; Nelson, et al. (1987) Molec. Cell. Biol. 7, 4125-4129) and also contains elements that allow packaging of the construct into virions. Other materials suitable for the practice of the present invention include an optimized mix of packaging plasmids (e.g., pLP1, pLP2, and pLP/VSVG) which may supply the structural and replication proteins in trans that are required to produce a recombinant retrovirus. In some embodiments, the present invention provides a cell line (e.g., 293FT), which allows production of the lentivirus following cotransfection of the expression plasmid and the plasmids in the packaging mix. In some embodiments, the present invention provides a control expression plasmid containing the lacZ gene which, when packaged into virions and transduced into a mammalian cell line, expresses β-galactosidase.

Using the materials and methods of the present invention (e.g., the ViraPower™ Lentiviral Expression System) to facilitate retroviral-based expression of the gene of interest provides the following advantages: 1) generates an HIV-1-based lentivirus that effectively transduces both dividing and non-dividing mammalian cells, thus broadening the potential applications beyond those of traditional Moloney Leukemia Virus (MoMLV)-based retroviral systems (Naldini, 1998); 2) efficiently delivers the gene of interest to mammalian cells in culture or in vivo (Dull et al., 1998); 3) provides stable, long-term expression of a target gene beyond that offered by traditional adenoviral-based systems (Dull et al., 1998; Naldini et al., 1996); 4) produces a pseudotyped virus with a broadened host range (Yee et al., 1994); and 5) includes multiple features designed to enhance the biosafety of the system.

One of skill in the art can use the teachings provided herein to: co-transfect the vectors described herein (e.g., pLenti6/V5-based expression vector) and the ViraPower™ Packaging Mix into the 293FT cell line to produce a lentiviral stock; titer the lentiviral stock; use the lentiviral stock to transduce a mammalian cell line of choice; assay for “transient” expression of one or more recombinant proteins encoded by the transduced vector; and/or generate a stably transduced cell line, if desired.

Additional details and instructions to generate an expression vector using pLenti6/V5-D-TOPO® or pLenti6/V5-DEST™ are available (e.g., pLenti6/V5 Directional TOPO® Cloning Kit manual, catalog no. K4955-10, version B, or pLenti6/V5-DEST™ GATEWAY™ Vector Pack manual, catalog nos. V496-10, V498-10, and V499-10, version C, Invitrogen Corporation, Carlsbad, Calif.). For instructions to culture and maintain the 293FT producer cell line, see Example 13 below.

Expression systems of the present invention (e.g., the ViraPower™ Lentiviral Expression System) facilitate highly efficient, in vitro or in vivo delivery of a target gene to dividing and non-dividing mammalian cells using a replication-incompetent lentivirus. Based on the lentikat™ system developed by Cell Genesys (Dull et al., 1998), the ViraPower™ Lentiviral Expression System possesses features which enhance its biosafety while allowing high-level gene expression in a wider range of cell types than traditional retroviral systems.

One component of the systems of the invention is an expression vector (e.g., a pLenti6/V5-based expression vector) into which the sequence of interest (e.g., encoding a gene of interest) will be cloned. Expression of the sequence of interest is controlled by a promoter of choice, for example, the human cytomegalovirus (CMV) promoter. The vector also contains the elements required to allow packaging of the expression construct into virions (e.g. 5′ and 3′ LTRs, Ψ packaging signal).

Another component of a system of the invention is one or more plasmids encoding the activities necessary for packaging the RNA produced from the expression vector (e.g., the ViraPower™ Packaging Mix that contains an optimized mixture of the three packaging plasmids, pLP1, pLP2, and pLP/VSVG). These plasmids supply the helper functions as well as structural and replication proteins in trans required to produce the lentivirus.

An optional component of the system is an optimized cell line (e.g., the 293FT producer cell line) that may stably express the SV40 large T antigen.

Expression of the SV40 large T antigen may be under the control of any promoter known in the art, for example, the human CMV promoter. Expression of the large T antigen facilitates optimal production of virus.

In an embodiment, plasmids containing the packaging activities (e.g., the ViraPower™ Packaging Mix) and an expression plasmid (e.g., the pLenti6/V5 vector containing a sequence of interest) may be co-transfected into a suitable host cell line (e.g., 293FT cells) to produce a replication-incompetent lentivirus, which can then be transduced into the mammalian cell line of interest. Once the lentivirus enters the target cell, the viral RNA is reverse-transcribed, actively imported into the nucleus (Lewis et al. 1994; Naldini et al., 1999), and stably integrated into the host genome (Buchschacher et al., 2000; Luciw, (1996) In Fields Virology, B. N. Fields, et al. eds. (Philadelphia, Pa.: Lippincott-Raven Publishers), pp. 1881-1975). Once the lentiviral construct has integrated into the genome, transient expression of a recombinant protein can be assayed or blasticidin selection can be used to generate a stable cell line for long-term expression.

Most retroviral vectors are limited in their usefulness as gene delivery vehicles by their restricted tropism and generally low titers. In the systems of the invention (e.g., the ViraPower™ Lentiviral Expression System), this limitation has been overcome by use of the G glycoprotein gene from Vesicular Stomatitis Virus (VSV-G) as a pseudotyping envelope, thus allowing production of a high titer lentiviral vector with a significantly broadened host cell range (Bums et al., (1993) Proc. Natl. Acad. Sci. USA 90, 8033-8037, Emi et al., (1991) J. Virol. 65, 1202-1207, Yee et al., 1994). Cell Lines and Cell Types Tested

| Cell Line or | | |
|---|---|---|
| Cell Type | Description | Condition Tested |
| 293 | Human embryonic kidney | Actively dividing |
| | (Graham et al., (1977) | |
| | J. Gen. Virol. 36, | |
| | 59-74) | |
| HT1080 | Human fibrosarcoma | Actively dividing |
| | (Rasheed et al., | Aphidicolin-arrested |
| | (1974) Cancer 33, | (at the G1/S transi- |
| | 1027-1033) | tion) |
| HeLa | Human cervical adeno- | Actively dividing |
| | carcinoma | |
| CHO-K1 | Chinese hamster ovary | Actively dividing |
| | (Kao et al., (1968) | |
| | Proc. Natl. Acad. Sci. | |
| | USA 60, 1275-1281) | |
| Primary foreskin | Human foreskin | Contact inhibited, |
| fibroblasts | | growth-arrested (in |
| | | G₀) |
| Primary hippo- | Rat neuronal tissue | Non-dividing, post- |
| campal neurons | | mitotic |
| Primary cortical | Rat neuronal tissue | Non-dividing, post- |
| neurons | | mitotic |

The present invention is suitable for in vivo gene delivery applications. Many groups have successfully used lentiviral vectors to express a target gene in tissues including brain, retina, pancreas, muscle, liver, and skin (Gallichan et al., (1998) Human Gene Therapy 9, 2717-2726; Kafri et al., (1997) Nature Genetics 17, 314-317; Miyoshi et al., (1997) Proc. Natl. Acad. Sci. USA 94, 10319-10323; Naldini, (1998) Curr. Opin. Biotechnol. 9, 457-463; Pfeifer et al., (2001) Proc. Natl. Acad. Sci. USA 98, 11450-11455; Pfeifer et al., (2001) Mol. Ther. 3, 319-322; Takahashi et al., (1999) J. Virol. 73, 7812-7816). For more information about target genes that have been successfully expressed in vivo using lentiviral-based vectors, refer to the references above as well as the following additional references (Baek et al., 2001; Dull et al., 1998; Park et al., 2001; Peng et al., 2001).

The systems of the invention (e.g., the ViraPower™ Lentiviral Expression System) are third-generation systems based on lentiviral vectors developed by Dull et al. (1998). These third-generation lentiviral systems include a significant number of safety features designed to enhance their biosafety and to minimize their relation to the wild-type, human HIV-1 virus. These safety features are discussed below.

The expression vector (pLenti6/V5-D-TOPO® or pLenti6/V5-DEST™) contains a deletion in the 3′ LTR (ΔU3) that does not affect generation of the viral genome in the producer cell line, but results in “self-inactivation” of the lentivirus after transduction of the target cell (Yee et al., 1987; Yu et al., 1986; Zufferey et al., 1998). Once integrated into the transduced target cell, the lentiviral genome is no longer capable of producing packageable viral genome.

The number of genes from HIV-1 that are used in the system has been reduced to three (i.e. gag, pol, and rev).

The VSV-G gene from Vesicular Stomatitis Virus is used in place of the HIV-1 envelope (Bums et al., 1993; Emi et al., 1991; Yee et al., 1994).

Genes encoding the structural and other components required for packaging the viral genome are separated onto four plasmids. All four plasmids have been engineered not to contain any regions of homology with each other to prevent undesirable recombination events that could lead to the generation of a replication-competent virus (Dull et al., 1998).

Although the three packaging plasmids allow expression in trans of proteins required to produce viral progeny (e.g. gal, pol, rev, env) in the 293FT producer cell line, none of them contain LTRs or the Ψ packaging sequence. This means that none of the HIV-1 structural genes are actually present in the packaged viral genome, and thus, are never expressed in the transduced target cell. No new replication-competent virus can be produced.

The lentiviral particles produced in this system are replication-incompetent and only carry the gene of interest. No other viral species are produced.

Expression of the gag and pol genes from pLP1 has been rendered Rev-dependent by virtue of the HIV-1 RRE in the gag/pol mRNA transcript. Addition of the RRE prevents gag and pol expression in the absence of Rev (Dull et al., 1998).

A constitutive promoter (RSV promoter, Gorman et al. (1982). Proc. Natl. Acad. Sci. USA 79, 6777-6781) has been placed upstream of the 5′ LTR in the pLenti6/V5 expression vector to offset the requirement for Tat in the efficient production of viral RNA (Dull et al., 1998).

Despite the inclusion of the safety features discussed above, the lentivirus produced with the systems of the invention can still pose some biohazardous risk since they can transduce primary human cells. For this reason, published guidelines for BL-2 should be followed. Furthermore, exercise extra caution when creating lentivirus carrying potential harmful or toxic genes (e.g. activated oncogenes).

For more information about the BL-2 guidelines and lentivirus handling, refer to the document, “Biosafety in Microbiological and Biomedical Laboratories”, 4^{th} Edition, published by the Centers for Disease Control (CDC).

The diagram in FIG. 35 describes the general steps required to express a sequence of interest using an exemplary system of the invention.

The present of the invention is designed to help one skilled in the art create a lentivirus to deliver and express a gene of interest in mammalian cells. For more information about retroviral biology and eukaryotic cell culture, refer to the following published reviews: Buchschacher et al. (2000); Luciw (1996); Naldini (1999), Naldini (1998), and Yee (1999) Retroviral Vectors. In The Development of Human Gene Therapy, T. Friedmann, ed. (Cold Spring Harbor, N.Y.: Cold Spring Harbor Laboratory Press), pp. 21-45.

The pLP1, pLP2, pLP/VSVG plasmids are provided in an optimized mixture to facilitate viral packaging of an expression vector (e.g., a pLenti6/V5-based expression vector) following cotransfection into 293FT producer cells. The amount of the packaging mix (195 μg) and Lipofectamine™ 2000 transfection reagent (0.75 ml) supplied in the kit is sufficient to perform 20 cotransfections in 10 cm plates using the recommended protocol describe herein. To use the ViraPower™ Packaging Mix, resuspend in 195 μl of sterile water to obtain a 1 μg/μl stock.

A pLenti6/V5 expression vector containing a gene of interest in pLenti6/V5-D-TOPO® or pLenti6/V5-DEST™ can be generated using methods described herein. Once an expression construct has been created, use any method of choice to prepare purified plasmid DNA. Plasmid DNA for transfection into eukaryotic cells must be clean and free from phenol and sodium chloride as contaminants may kill the cells, and salt will interfere with lipid complexing, decreasing transfection efficiency. Suitable methods for isolating plasmid of sufficient purity include the S.N.A.P.™ MidiPrep Kit (Invitrogen Corporation, Carlsbad, Calif., Catalog no. K1910-01) and CsCl gradient centrifugation.

Resuspend the purified expression plasmid (e.g., a pLenti6/V5 expression plasmid) containing a gene of interest in sterile water or TE, pH 8.0 at a concentration ranging from 0.1-3.0 μg/μl. 3 μg of expression plasmid may be used for each transfection.

A suitable host cell line is the human 293FT cell line available from Invitrogen Corporation, Carlsbad, Calif. and supplied with the ViraPower™ Lentiviral Expression kits (Naldini et al., 1996). The 293FT cell line, a derivative of the 293F cell line, stably and constitutively expresses the SV40 large T antigen from pCMVSPORT6TAg.neo and must be maintained in medium containing Geneticin®.

Before a stably transduced cell line expressing a gene of interest can be created, a lentiviral stock (containing the packaged expression construct) must be created by cotransfecting the optimized packaging plasmid mix and an expression vector (e.g., a pLenti6/V5-based expression vector) into a suitable host cell line (e.g., the 293FT cell line).

One suitable protocol for generating a lentiviral stock employs the following materials: ViraPower™ Packaging Mix (supplied with the kit; resuspend in 195 μl of sterile water to a concentration of 1 μg/μl); pLenti6/V5 expression vector containing a gene of interest (0.1-3.0 μg/μl in sterile water or TE, pH 8.0); pLenti6/V5-based positive control vector (supplied with the kit; resuspend in sterile water to a concentration of 1 μg/μl); 293FT cells cultured in the appropriate medium (see Example 13); Lipofectamine™ 2000 transfection reagent (supplied with the kit; store at +4° C. until use); Opti-MEMO® I Reduced Serum Medium (pre-warmed; see below); Fetal bovine serum (FBS); sterile 10 cm tissue culture plates (one each for the lentiviral construct, positive control, and negative control); sterile tissue culture supplies; 15 ml sterile, capped, conical tubes; and cryovials.

Each pLenti6/V5-based expression vector kit includes a positive control vector for use as an expression control (e.g. pLenti6/V5-GW/lacZ). It is recommended that the positive control vector be included in a cotransfection experiment to generate a control lentiviral stock that may be used to help optimize expression conditions in a mammalian cell line of interest.

Any suitable transfection reagent may be used to introduce the plasmids into the producer cell line. One suitable transfection reagent is Lipofectamine™ 2000 reagent (Ciccarone et al., (1999) Focus 21, 54-55). This reagent is a proprietary, cationic lipid-based formulation suitable for the transfection of nucleic acids into eukaryotic cells. Using Lipofectamine™ 2000 to transfect 293FT cells offers the following advantages: provides the highest transfection efficiency in 293FT cells; DNA-Lipofectamine™ 2000 complexes can be added directly to cells in culture medium in the presence of serum; and removal of complexes or medium change or addition following transfection are not required, although complexes can be removed after 4-6 hours without loss of activity.

To facilitate optimal formation of DNA-Lipofectamine™ 2000 complexes, a reduced serum medium (e.g., Opti-MEM® I Reduced Serum Medium available from Invitrogen Corporation, Carlsbad, Calif.) may be used.

Lentiviral stocks in 293FT cells produced using the optimized transfection conditions described herein. The amount of lentivirus produced using these recommended conditions (at a titer of 1×10⁵ to 1×10⁷ transducing units (TU)/ml) is generally sufficient to transduce 1×10⁶ to 1×10⁸ cells at a multiplicity of infection (MOI)=1.

| Condition | Amount | |
|---|---|---|
| Tissue culture plate size | 10 | cm |
| | (one per lentiviral construct) | |
| Number of 293FT cells to 5 × 10⁶ cells (see below) | | |
| transfect | | |
| Amount of ViraPower ™ | 9 | μg |
| Packaging Mix | (9 μl of 1 μg/μl stock) | |
| Amount of pLenti6/V5 | 3 | μg |
| expression plasmid | | |
| Amount of Lipofectamine ™ | 36 | μl |
| 2000 | | |

293FT cells should be plated 24 hours prior to transfection in complete medium, and should be 90-95% confluent on the day of transfection. Make sure that cells are healthy at the time of plating.

Follow the procedure below to cotransfect 293FT cells. Remember that the cells may be kept in culture medium during transfection. A positive control and a negative control (no DNA, no Lipofectamine™ 2000) are recommended to help evaluate results.

The day before transfection, trypsinize and count the 293FT cells, plating them at 5×10⁶ cells per 10 cm plate. Plate cells in 10 ml of normal growth medium containing serum.

On the day of transfection, remove the culture medium from the 293FT cells and replace with 5 ml of normal growth medium containing serum (or Opti-MEM® I Medium containing serum). Do not include antibiotics.

Prepare DNA-Lipofectamine™ 2000 complexes for each transfection sample by performing the following: Dilute 9 μg of the optimized packaging mix and 3 μg of pLenti6/V5 expression plasmid DNA (12 μg total) in 1.5 ml of Opti-MEM® I Medium without serum. Mix gently. Mix Lipofectamine™ 2000 gently before use, then dilute 36 μl in 1.5 ml of Opti-MEM® I Medium without serum. Mix gently and incubate for 5 minutes at room temperature. After the 5 minute incubation, combine the diluted DNA with the diluted Lipofectamine™ 2000. Mix gently. Incubate for 20 minutes at room temperature to allow the DNA-Lipofectamine™ 2000 complexes to form. The solution may appear cloudy, but this will not impede the transfection.

Add the DNA-Lipofectamine™ 2000 complexes dropwise to each plate. Mix gently by rocking the plate back and forth. Incubate the cells overnight at 37° C. in a CO₂ incubator.

The next day, remove the medium containing the DNA-Lipofectamine™ 2000 complexes and replace with complete culture medium (i.e. D-MEM containing 10% FBS, 2 mM L-glutamine, and 1% penicillin/streptomycin). Expression of the VSV G glycoprotein causes 293FT cells to fuise, resulting in the appearance of multinucleated syncitia. This morphological change is normal and does not affect production of the lentivirus.

Harvest virus-containing supernatants 48-72 hours posttransfection by removing medium to a 15 ml sterile, capped, conical tube. Minimal differences in viral yield are observed whether supernatants are collected 48 or 72 hours posttransfection. Remember that the supernatant contains infectious virus at this stage. Follow the recommended guidelines for working with BL-2 organisms.

Centrifuge at 3000 rpm for 15 minutes at +4° C. in a table top clinical centrifuge.

Perform A filtration step, if desired. Pipet viral supernatants into cryovials in 1 ml aliquots. Store viral. stocks at −80° C.

If the lentiviral construct is to be used for in vivo applications or if the stock is to be concentrated to obtain a higher titer, filtering the viral supernatant through a sterile, 0.45 μm low protein binding filter after the low-speed centrifugation step is recommended. A suitable filter is the Millex-HV 0.45 μm PVDF filter (Millipore, Catalog no. SLHVR25LS).

Place viral stocks at −80° C. for long-term storage. Repeated freezing and thawing is not recommended as it may result in loss of viral titer. When stored properly, viral stocks of an appropriate titer should be suitable for use for up to one year. After long-term storage, it is recommended that the titer of the viral be determined before transducing a cell line of interest.

It is possible to scale up the cotransfection experiment to produce a larger volume of lentivirus, if desired. For example, the cotransfection experiment may scaled up from a 10 cm plate to a T225 flask and up to 50 ml of viral supernatant may be harvested. To scale up, increase the number of cells plated and the amounts of DNA, Lipofectamine™ 2000, and medium used in proportion to the difference in surface area of the culture vessel.

Before proceeding to transduce the mammalian cell line of interest and express a recombinant protein, it is recommended that the titer of the lentiviral stock be determined. While this procedure is not required for some applications, it is necessary to control the number of integrated copies of the lentivirus or to generate reproducible expression results.

To determine the titer of a lentiviral stock: prepare 10-fold serial dilutions of the lentiviral stock; transduce the different dilutions of lentivirus into the mammalian cell line of choice in the presence of Polybrene®; select for stably transduced cells using blasticidin; and stain and count the number of blasticidin-resistant colonies in each dilution.

A number of factors can influence viral titers. One factor is the size of the sequence of interest inserted into the expression vector. Titers will generally decrease as the size of the insert increases. The size of the wild-type HIV-1 genome is approximately 10 kb. Since the size of the elements required for expression from pLenti6/V5 totals approximately 4 kb, the size of the gene of interest should theoretically not exceed 6 kb for efficient packaging.

Other factors that may influence viral titer are the characteristics of the cell line used for titering, the age of the lentiviral stock, the number of freeze thaw cycles that the stock has undergone, and the storage conditions of the stock. Viral titers may decrease with long-term storage at −80° C. If a lentiviral stock has been stored for 6 months to 1 year, it is recommended that the titer be determined prior to use in an expression experiment. Viral titers can decrease as much as 10% with each freeze/thaw cycle. Lentiviral stocks should be aliquotted and stored at −80° C.

The titer of a lentiviral stock may be determined using any mammalian cell line of choice. Generally, it is recommended that the same mammalian cell line be used to titer the lentiviral stock will be used to perform expression studies. However, in some instances, a different cell line may be used to titer the lentivirus (e.g. if performing expression studies in a non-dividing cell line or a primary cell line). In these cases, suitable cell lines with which to titer the lentivirus are those that: grow as an adherent cell line; are easy to handle; exhibit a doubling time in the range of 18-25 hours; and are non-migratory. An example of a suitable cell is the HT1080 human fibrosarcoma cell line (ATCC, Catalog no. CCL-121) for titering purposes, but other cell lines including HeLa and NIH3T3 are also suitable.

The titer of a lentiviral construct may vary depending on which cell line is chosen. If more than one lentiviral construct are to be used, it is recommended that the titer all of the lentiviral constructs be determined using the same cell line.

The pLenti6/V5 expression construct contains the blasticidin resistance gene (bsd) (Kimura et al., (1994) Biochim. Biophys. ACTA 1219, 653-659, Izumi, et al. (1991) Exp. Cell Res. 197, 229-233.) to allow for blasticidin selection of mammalian cells that have stably transduced the lentiviral construct (Takeuchi et al., (1958) The Journal of Antibiotics, Series A 11, 1-5; Yamaguchi et al., (1965) J. Biochem (Tokyo) 57, 667-677.

Since stably transduced cells are selected using blasticidin, the minimum concentration of blasticidin required to kill untransduced cells must be determined (i.e. perform a kill curve experiment). Typically, concentrations ranging from 2-10 μg/ml blasticidin are sufficient to kill most untransduced mammalian cell lines. For any given cell line of interest, a range of concentrations should be tested (see protocol below) to ensure that the minimum concentration necessary for the cell line is used. A suitable method to determine the appropriate concentration of blasticidin for a given cell line follows.

Prepare a set of 6 plates. Plate cells at approximately 25% confluence. Allow cells to adhere overnight.

The next day, substitute culture medium with medium containing varying concentrations of blasticidin (e.g., 0, 2, 4, 6, 8, 10 μg/ml blasticidin).

Replenish the selective media every 3-4 days, and observe the percentage of surviving cells.

Determine the appropriate concentration of blasticidin that kills the cells within 10 days after addition of blasticidin.

To determine the titer of a lentiviral construct, the following materials will be needed: the lentiviral stock (store at −80° C. until use); an adherent mammalian cell line of choice; complete culture medium for the cell line; hexadimethrine bromide (Polybrene®; Sigma, Catalog no. H9268; 6-well tissue culture plates; blasticidin (10 mg/ml stock solution); crystal violet (Sigma, Catalog no. C3886; prepare a 1% crystal violet solution in 10% ethanol); and Phosphate-Buffered Saline (PBS; Invitrogen, Catalog no. 10010-023).

When adding virus to mammalian cells, Polybrene® is included to enhance transduction of the virus into the cell. To use Polybrene®: prepare a 6 mg/ml stock solution in deionized, sterile water; filter-sterilize and dispense 1 ml aliquots into sterile microcentrifuge tubes; store at −20° C. for long-term storage. Stock solutions may be stored at −20° C. for up to 1 year. Do not freeze/thaw the stock solution more than 3 times as this may result in loss of activity. The working stock may be stored at +4° C. for up to 2 weeks.

The media contains infectious virus and appropriate safety precautions should be taken. For example, perform all manipulations within a certified biosafety cabinet. Treat media containing virus with bleach. Treat used pipets, pipette tips, and other tissue culture supplies with bleach or dispose of as biohazardous waste. Wear gloves, a laboratory coat, and safety glasses or goggles when handling viral stocks and media containing virus.

Follow the procedure below to determine the titer of a lentiviral stock using the mammalian cell line of choice. At least one 6-well plate is used for every lentiviral stock to be titered (one mock well plus five dilutions). If a lentiviral stock of the pLenti6/V5-GW/lacZ positive expression control has been made, it is recommended that this stock be titered as well.

The day before transduction (Day 1), trypsinize and count the cells, plating them such that they will be 30-50% confluent at the time of transduction. Incubate cells at 37° C. overnight.

Example: When using HT1080 cells, generally plate 2×10⁵ cells per well in a 6-well plate.

On the day of transduction (Day 2), thaw the lentiviral stock and prepare 10-fold serial dilutions ranging from 10⁻² to 10⁻⁶. For each dilution, dilute the lentiviral construct into complete culture medium to a final volume of 1 ml. Do not vortex. A wider range of serial dilutions (10⁻² to 10⁻⁸) may be used, if desired.

Remove the culture medium from the cells. Mix each dilution gently by inversion and add to one well of cells (total volume=1 ml).

Add Polybrene® to each well to a final concentration of 6 μg/ml. Swirl the plate gently to mix. Incubate at 37° C. overnight.

The following day (Day 3), remove the media containing virus and replace with 2 ml of complete culture medium.

The following day (Day 4), remove the medium and replace with complete culture medium containing the appropriate amount of blasticidin to select for stably transduced cells.

Remove medium and replace with fresh medium containing blasticidin every 3-4 days.

After 10-12 days of selection (day 14-16), no live cells in the mock well and discrete blasticidin-resistant colonies in one or more of the dilution wells should be seen. Remove the medium and wash the cells with 2 ml of PBS. Repeat the wash.

Add 1 ml of crystal violet solution and incubate for 10 minutes at room temperature.

Remove the crystal violet stain and wash the cells with 2 ml of PBS. Repeat wash.

Count the blue-stained colonies and determine the titer of the lentiviral stock.

When titering Lenti6/V5 lentiviral stocks using HT1080 cells, generally titers ranging from 5×10⁵ to 2×10⁷ transducing units (TU)/ml are observed. If the titer of a lentiviral stock is less than 1×10⁵ TU/ml, a new lentiviral stock should be produced.

As an example, a Lenti6/V5-GW/lacZ lentiviral stock was generated using the protocols described herein. HT1080 cells were transduced with 10-fold serial dilutions of the lentiviral supernatant (10⁻² to 10⁻⁶ dilutions) or untransduced (mock) following the protocol described above. Forty-eight hours post-transduction, the cells were placed under blasticidin selection (10 μg/ml). After 10 days of selection, the cells were stained with crystal violet (see plate below), and colonies were counted. In the plate, the colony counts were: mock: no colonies; 10⁻² dilution: confluent; undeterminable, 10⁻³ dilution: confluent; undeterminable, 10⁻⁴ dilution: confluent; undeterminable, 10⁻⁵ dilution: 46, and 10⁻⁶ dilution: 5. Thus, the titer of this lentiviral stock is 4.8×10⁶ TU/ml (i.e. average of 46×10⁵ and 5×10⁶).

Once a lentiviral stock with a suitable titer has been generatd, the lentiviral construct may be transduced into the mammalian cell line of choice and assayed for expression of a recombinant protein. An assay for expression of a gene of interest may be conducted in the following ways:

1) Pool a heterogeneous population of cells and test for expression directly after transduction (i.e. “transient” expression). Note that 24-48 hours must elapse after transduction before harvesting cells to allow time for the lentivirus genome to reverse transcribe and integrate into the chromosomal DNA. Integration must take place before expression of the gene of interest can occur.

2) Select for stably transduced cells using blasticidin. This requires a minimum of 10-12 days after transduction, but allows generation of clonal cell lines that stably express the gene of interest.

Stable expression of a target gene typically may be observed for at least 6 weeks following transduction and selection.

To select for stably transduced cells, the minimum concentration of blasticidin required to kill the untransduced mammalian cell line must be determined as described above. If the titer of the lentiviral construct was determined in the same cell line used to perform stable expression experiment, then the same concentration of blasticidin may be used for selection as was used for titering.

To obtain optimal expression of a gene of interest, cells must be transduced with a suitable MOI of lentivirus. MOI is defined as the number of virus particles per cell and generally correlates with the number of integration events and as a result, expression. Typically, expression levels increase linearly as the MOI increases.

A number of factors can influence determination of an optimal MOI including the nature of the cell line (e.g. non-dividing vs. dividing cell type), its transduction efficiency, the application of interest, and the nature of the gene of interest. If transducing a lentiviral construct into a mammalian cell line of choice for the first time, a range of MOIs should be used (e.g. 0, 0.05, 0.1, 0.5, 1, 2, 5) to determine the MOI required to obtain optimal expression of the recombinant protein for a particular application.

In general, 80-90% of the cells in an actively dividing cell line (e.g. HT1080, HeLa, CHO-K1) express a target gene when transduced at an MOI of ˜1. Some non-dividing cell types transduce lentiviral constructs less efficiently. For example, only about 50% of the cells in a culture of primary human fibroblasts express a target gene when transduced at an MOI of ˜1. If transducing a lentiviral construct into a non-dividing cell type, it may be necessary to increase the MOI to achieve optimal expression levels for a recombinant protein.

If a Lenti6/V5-GW/lacZ control lentiviral construct has been constructed, it may be used to help determine the optimal MOI for a particular cell line and application. Once the Lenti6/V5-GW/lacZ lentivirus has been transduced into the mammalian cell line of choice, the gene encoding β-galactosidase will be constitutively expressed and can be easily assayed using standard techniques.

Remember that viral supernatants are generated by harvesting spent media containing virus from the 293FT producer cells. Spent media lacks nutrients and may contain some toxic waste products. If a large volume of viral supernatant is used to transduce a mammalian cell line (e.g. 1 ml of viral supernatant per well in a 6-well plate), note that growth characteristics or morphology of the cells may be affected during transduction. These effects are generally alleviated after transduction when the media is replaced with fresh, complete media.

It is possible to concentrate VSV-G pseudotyped retroviruses using a variety of methods without significantly affecting their transducibility. If the titer of a lentiviral stock is relatively low (less than 5×10⁵ TU/ml) and an experiment requires a large volume of viral supernatant (e.g. a relatively high MOI), the virus may be concentrated before proceeding to transduction. For details and guidelines to concentrate the virus, refer to published reference sources (Yee, 1999).

To transduce a selected cell line, the following materials will be required: a titered stock of virus (e.g., a Lenti6/V5 lentiviral stock) which should be stored at −80° C. until use; a cell line of choice (e.g., a mammalian cell line); complete culture medium for the cell line; hexadimethrine bromide (Polybrene®; 6 mg/ml stock solution); appropriately sized tissue culture plates for the intended application; and blasticidin (if selecting for stably transduced cells; 10 mg/ml stock solution).

Follow the procedure below to transduce the mammalian cell line of choice with a lentiviral construct.

Plate cells in complete media as appropriate for the intended application.

On the day of transduction (Day 1), thaw the lentiviral stock and dilute (if necessary) the appropriate amount of virus (at a suitable MOI) into fresh complete medium. Do not vortex.

Remove the culture medium from the cells. Mix the medium containing virus gently by pipetting and add to the cells.

Add Polybrene® to a final concentration of 6 μg/ml. Swirl the plate gently to mix. Incubate at 37° C. overnight. To reduce possible negative effects of transducing cells with undiluted viral stock, it is possible to incubate cells for as little as 6 hours prior to changing medium.

The following day (Day 2), remove the medium containing virus and replace with fresh, complete culture medium.

The following day (Day 3), perform one of the following: harvest the cells and assay for expression of the recombinant protein of interest if performing transient expression experiments; or remove the medium and replace with fresh, complete medium containing the appropriate amount of blasticidin to select for stably transduced cells.

Remove medium and replace with fresh medium containing blasticidin every 3-4 days until blasticidin-resistant colonies can be identified (generally 10-12 days after selection).

Pick at least 5 blasticidin-resistant colonies and expand each clone to assay for expression of the recombinant protein.

Note that integration of the lentivirus into the genome is random. Depending upon the influence of the surrounding genomic sequences at the integration site, varying levels of recombinant protein expression may be seen from different blasticidin-resistant clones. Testing at least 5 blasticidin-resistant clones and selecting the clone that provides the optimal expression of the recombinant protein of interest is recommended.

Any method of choice known to those skilled in the art may be used to detect a recombinant protein of interest including, but not limited to, functional analysis, immunofluorescence, or western blot. If the gene of interest is cloned in frame with an epitope tag, the recombinant protein may be detected in a western blot using an antibody to the epitope tag.

Below are listed some potential problems and possible solutions that may help troubleshoot cotransfection and titering experiments.

| Problem | Reason | Solution |
|---|---|---|
| Low viral | Low transfection | |
| titer | efficiency: | |
| | Poor quality of | Use the S.N.A.P. ™ |
| | pLenti6/V5 | MidiPrep Kit to |
| | plasmid DNA | prepare plasmid DNA. |
| | Unhealthy | |
| | 293FT cells; | Use healthy 293FT |
| | cells exhibit | cells; do not overgrow. |
| | low viability | Cells should be 90-95% |
| | | confluent at the |
| | | time of transfection. |
| | 293FT cells | Optimize such that |
| | plated too | plasmid DNA (in |
| | sparsely | □g):Lipofectamine ™ |
| | Plasmid | 2000 (in □1) ratio |
| | DNA:transfection | ranges from 1:2 to 1:3. |
| | reagent ratio | |
| | incorrect | |
| | Viral supernatant | Concentrate virus using |
| | too dilute | any method of choice |
| | | (Yee, 1999). |
| | Viral supernatant | DO NOT freeze/thaw |
| | frozen and thawed | viral supernatant more |
| | multiple times | than 3 times. |
| | Poor choice of | Use an adherent cell line |
| | titering cell line | with the characteristics |
| | | discussed herein. |
| | Gene of interest | Viral titers generally |
| | is large | decrease as the size of |
| | | the insert increases; |
| | | inserts larger than 6 kb |
| | | are not recommended. |
| | Gene of interest | Generation of constructs |
| | is toxic to cells | containing activated |
| | | oncogenes or potentially |
| | | harmful genes is not |
| | | recommended. |
| | Polybrene ® not | Transduce the lentiviral |
| | included during | construct into cells in |
| | transduction | the presence of Polybrene ®. |
| No colonies | Too much blasticidin | Determine the sensitivity |
| obtained | used for selection | of the cell line to |
| upon titering | | blasticidin by performing |
| | | a kill curve experiment. |
| | | Use the minimum concentra- |
| | | tion of blasticidin required |
| | | to kill the untransduced |
| | | cell line. |
| | Viral stocks stored | Aliquot and store stocks |
| | incorrectly | at −80° C. Do not |
| | | freeze/thaw more than |
| | | 3 times. |
| | Polybrene ® not | Transduce the lentiviral |
| | included during | construct into cells in |
| | transduction | the presence of Polybrene ®. |
| Titer | Too little blasticidin | Increase amount of |
| indeterminable; | used for selection | blasticidin used for |
| cells confluent | | selection. |
| | Viral supernatant | Titer lentivirus using |
| | not diluted | a wider range of 10-fold |
| | sufficiently | serial dilutions (e.g. |
| | | 10⁻² to 10⁻⁸). |

Below are listed some potential problems and possible solutions that may help troubleshoot transduction and expression experiment.

| Problem | Reason | Solution |
|---|---|---|
| No expression | Promoter | The lentiviral construct |
| | silencing | may integrate into a chromo- |
| | | somal region that silences |
| | | the CMV promoter controlling |
| | | expression of the gene of |
| | | interest. Screen several |
| | | blasticidin-resistant clones |
| | | and select the one that |
| | | demonstrates the highest |
| | | expression levels of the |
| | | recombinant protein. |
| | Viral stocks | Aliquot and store stocks |
| | stored | at −80° C. Do not freeze/ |
| | incorrectly | thaw more than 3 times. |
| Poor | Poor transduction | Transduce the lentiviral |
| expression | efficiency: | construct into cells in the |
| | Polybrene ® | presence of Polybrene ®. |
| | not included | Transduce the lentiviral |
| | during | construct into cells using |
| | transduction | a higher MOI. |
| | Non-dividing | |
| | cell type used | |
| | MOI too low | Transduce the lentiviral |
| | | construct into cells using |
| | | a higher MOI. |
| | Too much | Determine the sensitivity of |
| | blasticidin used | the cell line to blasticidin |
| | for selection | by performing a kill curve |
| | | experiment. Use the minimum |
| | | concen-tration of blasticidin |
| | | required to kill the untrans- |
| | | duced cell line. |
| | Cells harvested | Do not harvest cells until |
| | too soon after | at least 24-48 hours after |
| | transduction | transduction to allow reverse |
| | | transcription and integration |
| | | of the lentivirus into the |
| | | genome. |
| | Gene of interest | Generation of constructs |
| | is toxic to cells | containing activated oncogenes |
| | | or potentially harmful genes |
| | | is not recommended. |

Table 25 provides some of the characteristics of the vector pLP1. The complete sequence is provided as table 21. A plasmid map is provided as FIG. 37A.

TABLE 25

| Feature | Benefit |
|---|---|
| Human cytomegalovirus | Permits high-level expression of |
| (CMV) promoter | the HIV-1 gag and pol genes in |
| bases 1-747, | mammalian cells (Andersson et al., |
| TATA box bases | 1989; Boshart et al., 1985; |
| 648-651 | Nelson et al., 1987). |
| Human β-globin | Enhances expression of the gag |
| intron | and pol genes in mammalian cells. |
| bases 880-1320 | |
| HIV-1 gag coding | Encodes the viral core proteins |
| sequence | required for forming the structure |
| bases 1355-2857 | of the lentivirus (Luciw, 1996). |
| HIV-1 pol coding | Encodes the viral replication |
| sequence | enzymes required for replication |
| bases 2650-5661 | and integration of the lenti- |
| | virus (Luciw, 1996). |
| HIV-1 Rev response | Permits Rev-dependent expression |
| element (RRE) | of the gag and pol genes |
| bases 5686-5919 | |
| Human β-globin poly- | Allows efficient transcription |
| adenylation signal | termination and polyadenylation |
| bases 6072-6837 | of mRNA. |
| pUC origin of repli- | Permits high-copy replication |
| cation (ori) | and maintenance in E. coli. |
| bases 6995-7668 | |
| complementary strand | |
| Ampicillin (bla) | Allows selection of the plasmid |
| resistance gene | in E. coli. |
| bases 7813-8673 | |
| complementary strand | |
| bla promoter | |
| bases 8674-8772 | |
| complementary strand | |

Table 26 provides some of the characteristics of the vector pLP2. The complete sequence is provided as Table 22. A plasmid map is provided as FIG. 37B.

TABLE 26

| Feature | Benefit |
|---|---|
| RSV enhancer/promoter | Permits high-level expression |
| bases 1-271, TATA box | of the rev gene (Gorman et al., |
| bases 200-207, | 1982). |
| transcription initiation | |
| base 229 | |
| RSV UTR bases 230-271 | |
| HIV-1 Rev ORF | Encodes the Rev protein which |
| bases 391-741 | interacts with the RRE on pLP1 |
| | to induce Gag and Pol expression, |
| | and on the pLenti6/V5 expression |
| | vector to promote the nuclear |
| | export of the unspliced viral |
| | RNA for packaging into viral |
| | particles. |
| HIV-1 LTR polyadenylation | Allows efficient transcription |
| signal | termination and polyadenylation |
| bases 850-971 | of mRNA. |
| Ampicillin (bla) | Allows selection of the plasmid |
| resistance gene | in E. coli. |
| promoter bases 1916-2014 | |
| gene bases 2015-2875 | |
| pUC origin of replication | Permits high-copy replication |
| (ori) | and maintenance in E. coli. |
| bases 3020-3693 | |

Table 27 provides some of the characteristics of the vector pLP/VSVG. The complete sequence is provided as Table 23. A plasmid map is provided as FIG. 37C.

TABLE 27

| Feature | Benefit |
|---|---|
| Human CMV promoter | Permits high-level expression |
| bases 1-747 | of the VSV-G gene in mammalian |
| | cells (Andersson et al., 1989; |
| | Boshart et al., 1985; Nelson et |
| | al., 1987). |
| Human (β-globin intron | Enhances expression of the |
| bases 880-1320 | VSV-G gene in mammalian cells. |
| VSV G glycoprotein (VSV-G) | Encodes the envelope G glyco- |
| bases 1346-2881 | protein from Vesicular Stomatitis |
| | Virus to allow production of a |
| | pseudotyped retrovirus with |
| | a broad host range (Burns et al., |
| | 1993; Emi et al., 1991; Yee et |
| | al., 1994). |
| Human β-globin | Allows efficient transcription |
| polyadenylation signal | termination and polyadenylation |
| bases 3004-3769 | of mRNA. |
| pUC origin of replication | Permits high-copy replication |
| (ori) | and maintenance in E. coli. |
| bases 3927-4600 | |
| complementary strand | |
| Ampicillin (bla) resistance | Allows selection of the plasmid |
| gene | in E. coli. |
| gene bases 4745-5606 | |
| complementary strand | |
| promoter bases 5606-5704 | |
| complementary strand | |

### Example 11

### GATEWAY™-Adapted Destination Vector for Cloning and High-Level Expression in Mammalian Cells Using the ViraPower™ Lentiviral Expression System

ViraPower™ Lentiviral Expression Products

The pLenti6/V5-DEST , pLenti4/V5-DEST, and pLenti6/UbC/V5-DEST vectors are designed for use with the ViraPower™ Lentiviral Expression System available from Invitrogen Corporation, Carlsbad, Calif., which is discussed in some detail above. Depending on the vector chosen, the pLenti-DEST vectors are available with the human cytomegalovirus (CMV) immediate early promoter or the human ubiquitin C (UbC) promoter to control expression of the gene of interest, and the Zeocin™ resistance gene or the blasticidin resistance gene for selection in E. coli or mammalian cells.

Expression of a recombinant fusion protein can be detected using an antibody to the V5 epitope. Horseradish peroxidase (HRP) or alkaline phosphatase (AP)-conjugated antibodies allow one-step detection using chemiluminescent or colorimetric detection methods. A fluorescein isothiocyanate (FITC)-conjugated antibody allows one-step detection in immunofluorescence experiments. Suitable detection reagents for fusion proteins can be obtained from Invitrogen Corporation, Carlsbad, Calif., for example, Anti-V5 Antibody, catalog number R960-25, Anti-V5-HRP Antibody, catalog number R961-25, Anti-V5-AP Antibody, catalog number R962-25, Anti-V5-FITC Antibody, catalog number R963-25.

pLenti6/V5-DEST™ is an 8.7 kb vector adapted for use with the GATEWAY™ Technology, and is designed to allow high-level expression of recombinant fusion proteins in dividing and non-dividing mammalian cells using Invitrogen's ViraPower™ Lentiviral Expression System. A map of the vector is provided as FIG. 36A and the sequence of the vector is provided as Table 17.

The pLenti-DEST vectors contain the following features: Rous Sarcoma Virus (RSV) enhancer/promoter for Tat-independent production of viral mRNA in the producer cell line (Dull et al., 1998); modified HIV-1 5′ and 3′ Long Terminal Repeats (LTR) for viral packaging and reverse transcription of the viral mRNA (Dull et al., 1998; Luciw, 1996) (Note: The U3 region of the 3′ LTR is deleted (ΔU3) and facilitates self-inactivation of the 5′ LTR after transduction to enhance the biosafety of the vector (Dull et al., 1998)); HIV-1 psi (Ψ) packaging sequence for viral packaging (Luciw, 1996); HIV Rev response element (RRE) for Rev-dependent nuclear export of unspliced viral mRNA (Kjems et al., 1991, Proc. Natl. Acad. Sci. USA 88, 683-687; Malim et al., 1989, Nature 338, 254-257); human CMV or UbC promoter for constitutive expression of the gene of interest from a viral or cellular promoter, respectively; two recombination sites, attR1 and attR2, downstream of the CMV or UbC promoter for recombinational cloning of the gene of interest from an entry clone; chloramphenicol resistance gene (Cm^{R}) located between the two attR sites for counterselection; the ccdB gene located between the attR sites for negative selection; C-terminal V5 epitope for detection of the recombinant protein of interest (Southern et al., 1991, J. Gen. Virol. 72, 1551-1557); blasticidin (Izumi et al., 1991; Kimura et al., 1994; Takeuchi et al., 1958; Yamaguchi et al., 1965) or Zeocin™ (Drocourt et al., 1990, Nucleic Acids Res. 18, 4009; Mulsant et al., 1988, Somat. Cell Mol. Genet. 14, 243-252) resistance gene for selection in E. coli and mammalian cells; ampicillin resistance gene for selection in E. coli; and the pUC origin for high-copy replication of the plasmid in E. coli.

A control plasmid containing the lacZ gene is included with each pLenti-DEST vector for use as a positive expression control in the mammalian cell line of choice.

The pLenti4/V5-DEST and pLenti6/V5-DEST vectors use the human CMV immediate early promoter to allow high-level, constitutive expression of the gene of interest in mammalian cells (Andersson et al., 1989; Boshart et al., 1985; Nelson et al., 1987). The sequence of the pLenti4/V5-DEST plasmid is provided as Table 19. Although highly active in most mammalian cell lines, activity of the viral CMV promoter can be down-regulated in some cell lines due to methylation (Curradi et al., 2002, Mol. Cell. Biol. 22, 3157-3173), histone deacetylation (Rietveld et al., 2002, EMBO J. 21, 1389-1397), or both.

The pLenti6/UbC/V5-DEST vector uses the human UbC promoter to allow constitutive, but more physiological levels of expression from the gene of interest in mammalian cells (Marinovic et al., 2000, Biophys. Res. Comm. 274, 537-541). The sequence of the pLenti6/UbC/V5-DEST plasmid is provided as Table 20. When compared to the CMV promoter, the UbC promoter is generally 2-4 fold less active. The UbC promoter is not down-regulated, making it useful for transgenic studies (Gill et al., 2001, Gene Ther. 8, 1539-1546; Lois et al., 2002, Science 295, 868-872; Marinovic et al., 2000; Schorpp et al., 1996, Nuc. Acids Res. 24, 1787-1788; Yew et al., 2001, Mol. Ther. 4, 75-82). The human ubiquitin C (UbC) promoter (in pLenti6/UbC/V5-DEST) allows high-level expression of recombinant protein is most mammalian cell lines (Wulffet al., 1990, FEBS Lett. 261, 101-105) and in virtually all tissues tested in transgenic mice (Schorpp et al., 1996). The diagram below shows the features of the UbC promoter as described by Nenoi et al., 1996 Gene 175, 179-185.

GATEWAY™ is a universal cloning technology that takes advantage of the site-specific recombination properties of bacteriophage lambda (Landy, 1989) to provide a rapid and highly efficient way to move a gene of interest into multiple vector systems. To express a sequence of interest (e.g., a sequence encoding a polypeptide of interest) in mammalian cells using the GATEWAY™ technology, simply: clone the sequence of interest into a GATEWAY™ entry vector of choice to create an entry clone; generate an expression clone by performing an LR recombination reaction between the entry clone and a GATEWAY™ destination vector (e.g. pLenti4/V5-DEST, pLenti6/V5-DEST, or pLenti6/UbC/V5-DEST); and use the expression clone in the ViraPower™ Lentiviral Expression System.

For more detailed information about GATEWAY™ System, generating an entry clone, and performing the LR recombination reaction, refer to the GATEWAY™ Technology manual available from Invitrogen Corporation, Carlsbad, Calif.

The pLenti4/V5-DEST, pLenti6/V5-DEST, and pLenti6/UbC/V5-DEST vectors are supplied as supercoiled plasmids. Although the GATEWAY™ Technology Manual has previously recommended using a linearized destination vector for more efficient recombination, further testing at Invitrogen has found that linearization of pLenti6/V5-DEST™ is not required to obtain optimal results for any downstream application.

To propagate and maintain the pLenti4/V5-DEST, pLenti6/V5-DEST, or pLenti6/UbC/V5-DEST vectors, Library Efficiency® DB3.1™ Competent Cells (Catalog no. 11782-018) from Invitrogen Corporation, Carlsbad, Calif. are recommended for transformation. The DB3.1™ E. coli strain is resistant to CcdB effects and can support the propagation of plasmids containing the ccdB gene. To maintain integrity of the vector, select for transformants in media containing 50-100 μg/ml ampicillin and 15 μg/ml chloramphenicol. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the destination vector containing attP sites flanking the ccdB and spectinomycin resistance genes can be selected on ampicillin/spectinomycin-containing media. It has recently been found that the use of spectinomycin selection instead of chloramphenicol selection results in an increase in the number of colonies obtained on selection plates, indicating that use of the spectinomycin resistance gene may lead to an increased efficiency of cloning from that observed using cassettes containing the chloramphenicol resistance gene. Do not use general E. coli cloning strains including TOP10 or DH5α for propagation and maintenance as these strains are sensitive to CcdB effects.

To recombine a sequence of interest into pLenti4/V5-DEST, pLenti6/V5-DEST, or pLenti6/UbC/V5-DEST, an entry clone containing the sequence must be created. Many entry vectors including pENTR/D-TOPO® are available from Invitrogen Corporation, Carlsbad, Calif. to facilitate generation of entry clones.

pLenti4/V5-DEST, pLenti6/V5-DEST, and pLenti6/UbC/V5-DEST are C-terminal fusion vectors. To express a fusion polypeptide of a polypeptide encoded by a sequence of interest with the V5 epitope coding sequence present in the vector, a sequence of interest must contain an ATG initiation codon in the context of a Kozak translation initiation sequence for proper initiation of translation in mammalian cells (Kozak, 1987; Kozak, 1991; Kozak, 1990). An example of a Kozak consensus sequence is (G/A)NNATGG. Other sequences are possible, but the G or A at position −3 and the G at position +4 are the most critical for function (shown in bold). The ATG initiation codon is underlined. The reading frame of the polypeptide encoded by the sequence of interest must be in frame with the C-terminal tag containing the V5 epitope after recombination and the sequence of interest must not contain a stop codon in this reading frame. The C-terminal peptide containing the V5 epitope and the attB2 site will add approximately 4.5 kDa to the size of the polypeptide encoded by the sequence of interest.

Each entry clone contains attL sites flanking the gene of interest. Genes in an entry clone are transferred to the destination vector backbone by mixing the DNAs with the GATEWAY™ LR Clonase™ Enzyme Mix available from Invitrogen Corporation, Carlsbad, Calif. The resulting recombination reaction is then transformed into E. coli (e.g. TOP10 or DH5α™-T1^{R}) and the expression clone selected (e.g., using ampicillin and blasticidin). Recombination between the attR sites on the destination vector and the attL sites on the entry clone replaces the chloramphenicol (Cm^{R}) gene and the ccdB gene with the gene of interest and results in the formation of attB sites in the expression clone.

Any recA, endA E. coli strain including TOP10, DH5α™, or equivalent may be used for transformation. Do not transform the LR reaction mixture into E. coli strains that contain the F′ episome (e.g. TOP10F′). These strains contain the ccdA gene and will prevent negative selection with the ccdB gene.

When transforming E. coli with the recombination reaction (pLenti4/V5-DEST, pLenti6/V5-DEST, or pLenti6/UbC/V5-DEST× entry clone), unwanted recombination (less than 5%) between the 5′ and 3′ LTRs has been observed when transformants are selected on LB agar plates containing ampicillin. These events occur less frequently when selection is performed using 100 μg/ml ampicillin and an additional selection, for example, 50 μg/ml blasticidin for pLenti6/V5-DEST or pLenti6/UbC/V5-DEST or 25 μg/ml Zeocin™ for pLenti4/V5-DEST. For Zeocin™ to be active, the salt concentration of the bacterial medium must be <90 mM and the pH must be 7.5. Therefore, selection on LB agar plates containing 50-100 μg/ml ampicillin and an additiona selection agent is recommended. Note that transformed E. coli grow more slowly in LB media containing ampicillin and blasticidin, and may require slightly longer incubation times to obtain visible colonies. Transformants that contain a recombined plasmid generally give rise to larger colonies than those containing an intact plasmid.

The ccdB gene mutates at a very low frequency, resulting in a very low number of false positives. True expression clones will be chloramphenicol-sensitive and ampicillin- and blasticidin-resistant (for pLenti6 vectors) and ampicillin- and Zeocin™-resistant (for pLenti4/V5-DEST). Transformants containing a plasmid with a mutated ccdB gene will be ampicillin-, blasticidin- or Zeocin™-, and chloramphenicol-resistant, as appropriate. To check a putative expression clone, test for growth on LB plates containing 30 μg/ml chloramphenicol. A true expression clone should not grow in the presence of chloramphenicol.

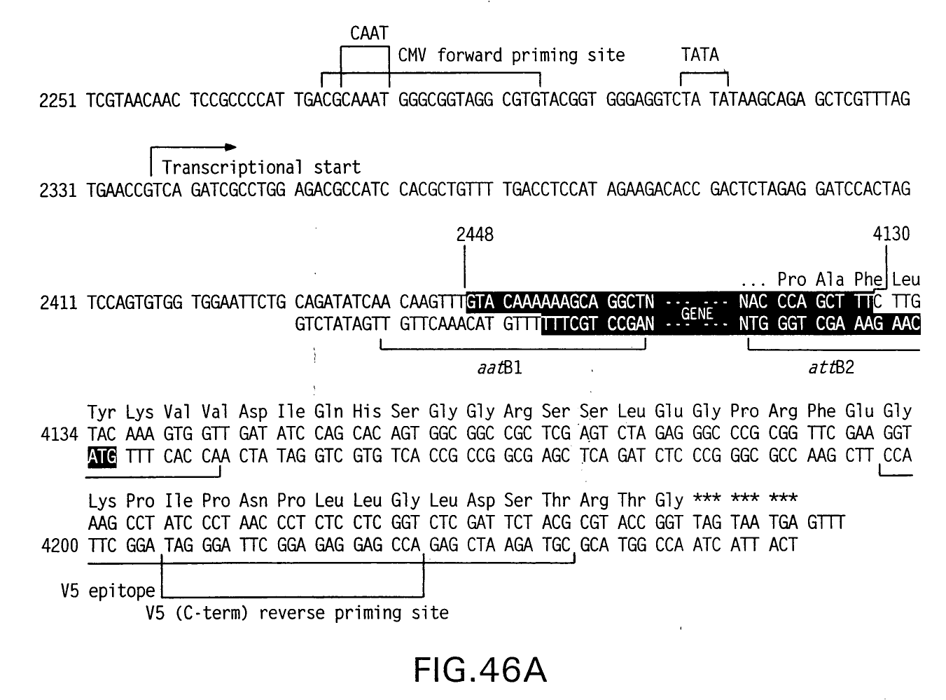

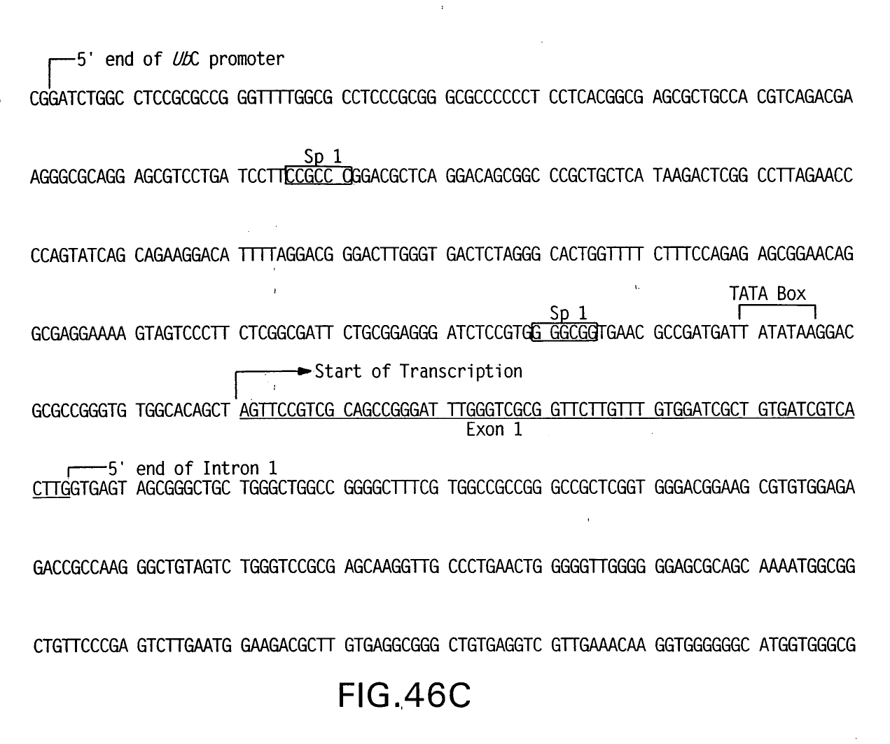

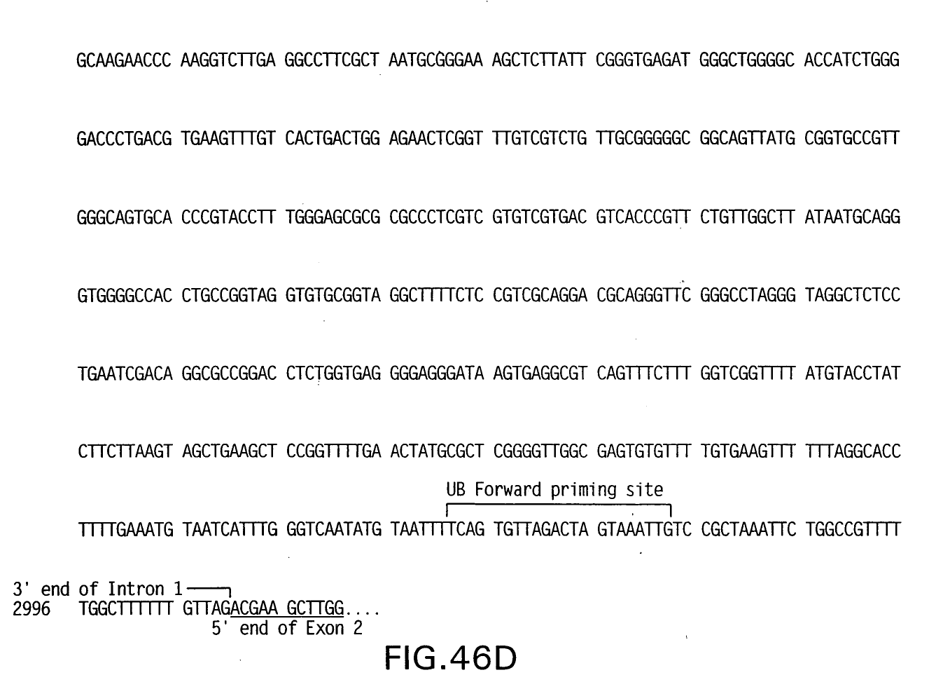

FIG. 46A provides a diagram of the recombination region of pLenti6/V5-DEST™ or pLenti4/V5-DEST after a recombination reaction with a sequence of interest. Shaded regions correspond to the sequence of interest transferred from the entry clone into the pLenti6/V5-DEST™ vector by recombination. Non-shaded regions are derived from the pLenti6/V5-DEST™ or pLenti4/V5-DEST vector. Bases 2448 and 4130 of the pLenti4/V5-DEST and pLenti6/V5-DEST™ sequences are marked. Restrictions sites are labeled to indicate the actual cleavage site.

FIG. 46B shows the recombination region of the expression clone resulting from pLenti6/UbC/V5-DEST× entry clone. Note that this diagram does not contain the complete sequence of the UbC promoter. For a diagram of the UbC promoter see FIG. 46C. Shaded regions in FIG. 46B correspond to those DNA sequences transferred from the entry clone into the pLenti6/UbC/V5-DEST vector by recombination. Non-shaded regions are derived from the pLenti6/UbC/V5-DEST vector. Bases 3079 and 4762 of the pLenti6/UbC/V5-DEST sequence are marked.

Once an expression clone has been generated in the pLenti6/V5-DEST backbone, maintain and propagate the plasmid in LB medium containing 50-100 μg/ml ampicillin. Addition of blasticidin is not required.

To confirm that a gene of interest is in frame with the C-terminal tag, sequence the expression construct, if desired. Refer to FIG. 46 for the location of the recommended primer binding sites (CMV or UbC forward priming site and V5(C-term) reverse priming site) to use to sequence the expression construct. To sequence a pLenti4/V5-DEST or pLenti6/V5-DEST construct, the CMV forward primer 5′-CGCAAATGGGCGGTAGGCGTG-3′ and V5(C-term) reverse primer 5′-ACCGAGGAGAGGGTTAGGGAT-3′ can be used. To sequence a pLenti6/UbC/V5-DEST construct, the UB forward primer 5′-TCAGTGTTAGACTAGTAAATTG-3′ and the V5(C-term) reverse primer 5′-ACCGAGGAGAGGGTTAGGGAT-3′ can be used.

Once purified plasmid DNA of the expression construct has been obtained, a viral stock can be prepared and used to transduce a cell line of choice as described above. Host cells containing the expression clone can be propagated in LB medium with ampicillin. It is not necessary to add an additional selection agent.

High salt and acidity or basicity inactivate Zeocin™. Therefore, it is recommended that the salt in bacterial medium be reduced and the pH adjusted to 7.5 to keep the drug active. Note that the pH and salt concentration do not need to be adjusted when preparing tissue culture medium containing Zeocin™. Store Zeocin™ at −20° C. and thaw on ice before use. Zeocin™ is light sensitive. Store the drug, and plates or medium containing drug, in the dark at +4° C. Culture medium containing Zeocin™ may be stored at +4° C. protected from exposure to light for up to 1 month. Wear gloves, a laboratory coat, and safety glasses or goggles when handling Zeocin™-containing solutions. Zeocin™ is toxic. Do not ingest or inhale solutions containing the drug.

The pLenti6/V5-DEST™ vector (8688 bp) contains the following features at the indicated locations. The locations of the features in the pLenti6/V5-DEST plasmid are as follows: RSV/5′ LTR hybrid promoter bases 1-410; RSV promoter bases 1-229; HIV-1 5′ LTR bases 230-410; 5′ splice donor base 520; HIV-1 psi (ψ) packaging signal bases 521-565; HIV-1 Rev response element (RRE) bases 1075-1308; 3′ splice acceptor base 1656; 3′ splice acceptor base 1684; CMV promoter bases 1809-2392; attR1 site: bases 2440-2564; Chloramphenicol resistance gene (Cm^{R}) bases 2673-3332; ccdB gene bases 3674-3979; attR2 site bases 4020-4144; V5 epitope bases 4197-4238; SV40 early promoter and origin bases 4293-4602; EM7 promoter bases 4657-4723; Blasticidin resistance gene bases 4724-5122; ΔU3/3′ LTR bases 5208-5442; ΔU3 bases 5208-5261; 3′ LTR: bases 5262-5442; SV40 polyadenylation signal bases 5514-5645; bla promoter bases 6504-6602; Ampicillin (bla) resistance gene bases 6603-7463; and pUC origin bases 7608-8281.

The pLenti4/V5-DEST vector(8634 nucleotides) contains the following features at the indicated locations: RSV/5′ LTR hybrid promoter bases 1-410; RSV promoter bases 1-229; HIV-1 5′ LTR bases 230-410; 5′ splice donor base 520; HIV-1 psi (ψ) packaging signal bases 521-565; HIV-1 Rev response element (RRE) bases 1075-1308; 3′ splice acceptor base 1656; 3′ splice acceptor base 1684; CMV promoter bases 1809-2392; attR1 site bases 2440-2564; Chloramphenicol resistance gene (Cm^{R}) bases 2673-3332; ccdB gene bases 3674-3979; attR2 site bases 4020-4144; V5 epitope bases 4197-4238; SV40 early promoter and origin bases 4293-4602; EM7 promoter bases 4621-4687; Zeocin™ resistance gene bases 4688-5062; ΔU3/3′ LTR bases 5154-5388; ΔU3 bases 5154-5207; 3′ LTR bases 5208-5388; SV40 polyadenylation signal bases 5460-5591; bla promoter bases 6450-6548; Ampicillin (bla) resistance gene bases 6549-7409; and the pUC origin bases 7554-8227.

The pLenti6/UbC/V5-DEST vector (9320 nucleotides) contains the following features at the indicated locations: RSV/5′ LTR hybrid promoter bases 1-410; RSV promoter bases 1-229; HIV-1 5′ LTR bases 230-410; 5′ splice donor base 520; HIV-1 psi (ψ) packaging signal bases 521-565; HIV-1 Rev response element (RRE) bases 1075-1308; 3′ splice acceptor base 1656; 3′ splice acceptor base 1684; UbC promoter bases 1798-3016; attR1 site bases 3072-3196; Chloramphenicol resistance gene (Cm^{R}) bases 3305-3964; ccdB gene bases 4306-4611; attR2 site bases 4652-4776; V5 epitope bases 4829-4870; SV40 early promoter and origin bases 4925-5234; EM7 promoter bases 5289-5355; Blasticidin resistance gene bases 5356-5754; ΔU3/3′ LTR bases 5840-6074; ΔU3 bases 5840-5893; 3′ LTR bases 5894-6074; SV40 polyadenylation signal bases 6146-6277; bla promoter bases 7136-7234; Ampicillin (bla) resistance gene bases 7235-8095; and the pUC origin bases 8240-8913.

### Example 12

### Five-Minute, Directional TOPO® Cloning of Blunt-End PCR Products into an Expression Vector for the ViraPower™ Lentiviral Expression System

The following protocol may be used to clone a nucleic acid segment using topoisomerase. Other protocols known to those skilled in the art are also suitable. An example of another suitable protocol may be found in the pENTR Directional TOPO® Cloning Kit manual available from Invitrogen Corporation, Carlsbad, Calif. (catalog number 25-0434).

| Step | | Action | |
|---|---|---|---|
| Design PCR Primers | | Include the 4 base pair sequences | |
| | | (CACC) necessary for directional | |
| | | cloning on the 5′ end of the | |
| | | forward primer. | |
| | | Design the primers such that a | |
| | | gene of interest will be optimally | |
| | | expressed and fused in frame with | |
| | | the V5 epitope tag, if desired. | |
| Amplify the Gene of | | Use a thermostable, proofreading DNA | |
| Interest | | polymerase and the PCR primers above | |
| | | to produce blunt-end PCR product. | |
| | | Use agarose gel electrophoresis to | |
| | | check the integrity of PCR product. | |
| Perform the TOPO ® | 1. | Set up the following TOPO ® | |
| Cloning Reaction | | Cloning reaction. | |
| | | Fresh PCR product | 0.5 to 4 μl |
| | | Salt Solution | 1 μl |
| | | Sterile water | add to a final |
| | | | volume of 5 μl |
| | | TOPO ® vector | 1 μl |
| | | Total volume | 6 μl |

| | | |
|---|---|---|
| | 2. | Mix gently and incubate for 5 |
| | | minutes at room temperature. |
| | 3. | Place on ice and proceed to trans- |
| | | form One Shot ® TOP 10 chemically |
| | | competent E. coli, below. |
| Transform One Shot ® | 1. | Add 2 μl of the TOPO ® Cloning |
| TOP 10 Chemically | | reaction into a vial of One Shot ® |
| Competent E. coli | | TOP 10 chemically competent E. coli |
| | | and mix gently. |
| | 2. | Incubate on ice for 5 to 30 minutes. |
| | 3. | Heat-shock the cells for 30 seconds |
| | | at 42° C. without shaking. |
| | | Immediately transfer the tube to ice. |
| | 4. | Add 250 μl of room temperature |
| | | SOC medium. |
| | 5. | Incubate at 37° C. for 1 hour |
| | | with shaking. |
| | 6. | Spread 50-200 μl of bacterial |
| | | culture on a prewarmed LB agar plate |
| | | containing 50-100 μg/ml ampi- |
| | | cillin and 50 μg/ml blasticidin, |
| | | and incubate overnight at 37° C. |

Using the Control PCR Template and the Control PCR Primers included with the kit to perform a control reaction is recommended. See the protocol below for details.

The pLenti6/V5 Directional TOPO® Cloning Kit is shipped on dry ice and contains two boxes. Upon receipt, store the boxes as detailed below.

| Box | Item | Storage |
|---|---|---|
| 1 | pLenti6/V5-D-TOPO ® Reagents | −20° C. |
| 2 | One Shot ® TOP 10 Chemically Competent E. coli | −80° C. |

pLenti6/V5-D-TOPO® reagents (Box 1) are listed below. Note that the user must supply a thermostable, proofreading polymerase and the appropriate PCR buffer.

| | | |
|---|---|---|
| Item | Concentration | Amount |

| | | | | |
|---|---|---|---|---|
| pLenti6/V5-D-TOPO ® | 10 | ng/μl linearized plasmid DNA in: | 20 | μl |
| | 50% | glycerol | | |
| | 50 | mM Tris-HCl, pH 7.4 (at 25° C.) | | |
| | 1 | mM EDTA | | |
| | 2 | mM DTT | | |
| | 0.1% | Triton X-100 | | |
| | 100 | μg/ml BSA | | |
| | 30 | μM bromophenol blue | | |
| dNTP Mix | 12.5 | mM dATP | 10 | μl |
| | 12.5 | mM dCTP | | |
| | 12.5 | mM dGTP | | |
| | 12.5 | mM dTTP | | |
| | | in water, pH 8 | | |
| Salt Solution | 1.2 | M NaCl | 50 | μl |
| | 0.06 | M MgCl2 | | |
| Sterile Water | — | | 1 | ml |
| CMV Forward | 0.1 | μg/μl in TE Buffer, pH 8 | 20 | μl |
| Sequencing Primer | | | | |
| V5(C-term) Reverse | 0.1 | μg/μl in TE Buffer, pH 8 | 20 | μl |
| Sequencing Primer | | | | |
| Control PCR Primers | 0.1 | μg/μl each in TE Buffer, pH 8 | 10 | μl |
| Control PCR Template | 0.1 | μg/μl in TE Buffer, pH 8 | 10 | μl |

| | | | |
|---|---|---|---|
| pLenti6/V5-GW/lacZ | Lyophilized in TE Buffer, pH 8 | 10 | μg |
| Expression Control | | | |
| Plasmid | | | |

Store Box 1 at −20° C.

The sequences of CMV Forward and V5(C-term) Reverse sequencing primers. Two micrograms of each primer are as follows:

| | |
|---|---|
| CMV Forward | 5′-CGCAAATGGGCGGTAGGCGTG-3′ |
| V5(C-term) Reverse | 5′-ACCGAGGAGAGGGTTAGGGAT-3′ |

TOP10 cells have the following genotype: F⁻ mcrA Δ(mrr-hsdRMS-mcrBC) Φ80lacZΔM15 ΔlacX74 recA1 deoR araD139 Δ(ara-leu)7697 galU galK rpsL (Str^{R}) endA1 nupG. Transformation efficiency is 1×10⁹ cfu/μg DNA and they should be stored at −80° C.

The pLenti6/V5-D-TOPO® vector is designed for use with the ViraPower™ Lentiviral Expression System available from Invitrogen Corporation, Carlsbad, Calif. Ordering information for the ViraPower™ Lentiviral Expression System and other ViraPower™ lentiviral support products and expression vectors is provided below. For more information, see the Invitrogen Corporation, Carlsbad, Calif. Web site.

| Item | Quantity | | Catalog no. |
|---|---|---|---|
| ViraPower ™ Lentiviral Directional | 1 | kit | K4950-00 |
| TOPO ® Expression Kit (includes | | | |
| ViraPower Lentiviral Support Kit | | | |
| and the 293FT Cell Line) | | | |
| ViraPower ™ Lentiviral GATEWAY ™ | 1 | kit | K4960-00 |
| Expression Kit | | | |
| pLenti6/V5-DEST ™ GATEWAY ™ | 6 | μg | V496-10 |
| Vector Pack | | | |
| ViraPower Lentiviral Support Kit | 20 | reactions | K4970-00 |
| (includes ViraPower Packaging Mix, | | | |
| Lipofectamine ™ 2000, and | | | |
| blasticidin) | | | |

| | | |
|---|---|---|
| 293FT Cell Line | 3 × 10⁶ cells | R700-07 |

Some of the reagents supplied in the pLenti6/V5 Directional TOPO® Cloning Kit as well as other reagents suitable for use with the kit are available separately from Invitrogen Corporation, Carlsbad, Calif. Ordering information for these reagents is provided below.

| Item | Quantity | | Catalog no. |
|---|---|---|---|
| One Shot ® TOP 10 Chemically | 10 | reactions | C4040-10 |
| Competent Cells | | | |
| | 20 | reactions | C4040-03 |
| One Shot ® TOP 10 Electro- | 10 | reactions | C4040-50 |
| competent Cells | | | |
| Ampicillin | 200 | mg | 11593-019 |
| Blasticidin | 50 | mg | R210-01 |
| ThermalAce ™ DNA Polymerase | 200 | units | E0200 |
| | 1000 | units | E1000 |
| Platinum ® Pfx DNA Polymerase | 100 | units | 11708-013 |
| Lipofectamine ™ 2000 | 0.75 | ml | 11668-027 |
| | 1.5 | ml | 11668-019 |

The pLenti6/V5 Directional TOPO® Cloning Kit combines the ViraPower™ Lentiviral Expression System with TOPO® Cloning technology to provide a highly efficient, rapid cloning strategy for insertion of blunt-end PCR products into a vector for expression in dividing and non-dividing mammalian cells. TOPO® Cloning requires no ligase, post-PCR procedures, or restriction enzymes.

pLenti6/V5-D-TOPO® is a 7.0 kb expression vector designed to facilitate rapid, directional TOPO® Cloning and high-level expression of PCR products in mammalian cells using the ViraPower™ Lentiviral Expression System (Catalog nos. K4950-00) available from Invitrogen Corporation, Carlsbad, Calif. Features of the vector include: RSV enhancer/promoter bases 1-229; HIV-1 5′ LTR bases 230-410; 5′ splice donor base 520; HIV-1 psi (ψ) packaging sequence bases 521-565; HIV-1 Rev response element (RRE) bases 1075-1308; 3′ splice acceptor base 1656; 3′ splice acceptor base 1684; CMV promoter bases 1809-2392; CMV forward priming site bases 2274-2294; directional TOPO® site bases 2431-2444; V5 epitope bases 2473-2514; V5(C-term) reverse priming site bases 2482-2502; SV40 early promoter and origin bases 2569-2878; EM7 promoter bases 2933-2999; Blasticidin resistance gene bases 3000-3398; ΔU3/HIV-1 3′ LTR bases 3485-3718; ΔU3: bases 3485-3537; Truncated HIV-1 3′ LTR bases 3538-3718; SV40 polyadenylation signal bases 3790-3921; bla promoter: bases 4780-4878; ampicillin (bla) resistance gene bases 4879-5739; and the pUC origin: bases 5884-6557.

The control plasmid, pLenti6/V5-GW/lacZ, may be used as a positive expression control in the mammalian cell line of choice.

The ViraPower™ Lentiviral Expression System facilitates highly efficient, in vitro or in vivo delivery of a target gene to dividing and non-dividing mammalian cells using a replication-incompetent lentivirus. Based on the lentikat™ system developed by Cell Genesys (Dull et al., 1998), the ViraPower™ Lentiviral Expression System possesses features which enhance its biosafety while allowing high-level gene expression in a wider range of cell types than traditional retroviral systems. To express a gene of interest in mammalian cells using the ViraPower™ Lentiviral Expression System:

1. TOPO® Clone a gene of interest into pLenti6/V5-D-TOPO® to create an expression construct.

2. Cotransfect the pLenti6/V5-D-TOPO® expression plasmid and the ViraPower™ Packaging Mix into the 293FT cell line to produce lentivirus.

3. Use the lentiviral stock to transduce the mammalian cell line of choice.

4. Assay for “transient” expression of the recombinant protein or generate a stable cell line using blasticidin selection.

Detailed protocols for creating recombinant lentiviruses are known (e.g., ViraPower™ Lentiviral Expression System manual, catalog nos. K4950-00, K4960-00, K4970-00, K4975-00, K49580-00, K49585-00, and K49590-00, version D, Invitrogen Corporation, Carlsbad, Calif.).

Directional joining of double-strand DNA using TOPO®-charged oligonucleotides occurs by adding a 3′ single-stranded end (overhang) to the incoming DNA (Cheng and Shuman, 2000, Mol. Cell. Biol. 20, 8059-8068.). This single-stranded overhang is identical to the 5′ end of the TOPO®-charged DNA fragment. The pLenti6/V5-D-TOPO® vector contains a 4 nucleotide overhang sequence.

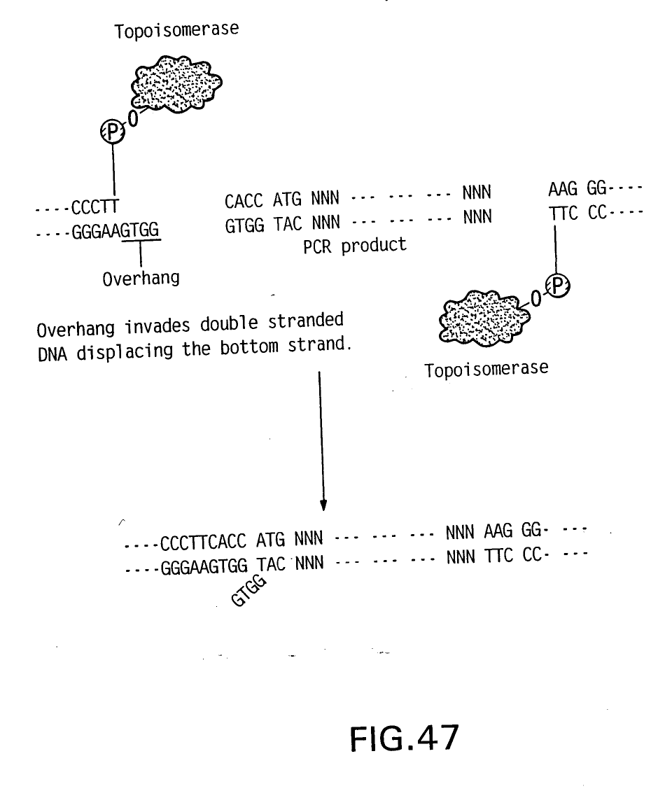

In this system, PCR products are directionally cloned by adding four bases to the forward primer (CACC). The overhang in the cloning vector (GTGG) invades the 5′ end of the PCR product, anneals to the added bases, and stabilizes the PCR product in the correct orientation. Inserts can be cloned in the correct orientation with efficiencies equal to or greater than 90%. A schematic representation of the process is shown in FIG. 47.

The design of the PCR primers to amplify a gene of interest is critical for expression. Consider the following when designing PCR primers: sequences required to facilitate directional cloning; sequences required for proper translation initiation of the PCR product; and whether or not a coding sequence contained by the PCR product is to be fused in frame with the C-terminal V5 epitope tag.

When designing a forward PCR primer, consider the points below.

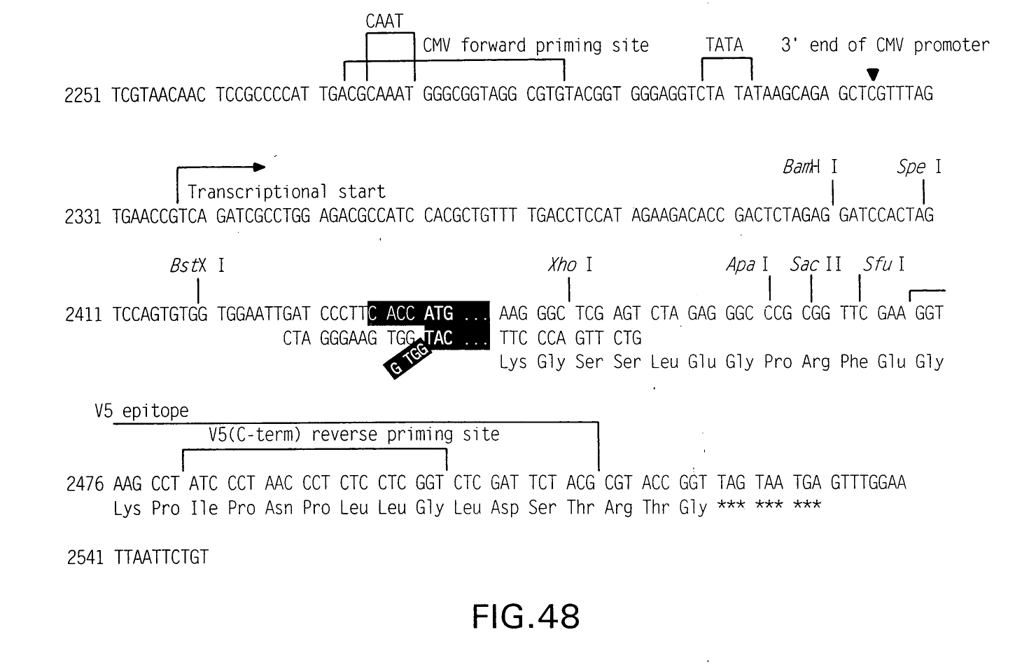

Refer to FIG. 48 for a diagram of the TOPO® Cloning site for pLenti6/V5-D-TOPO®.

To enable directional cloning, the forward PCR primer MUST contain the sequence, CACC, at the 5′ end of the primer. The 4 nucleotides, CACC, base pair with the overhang sequence, GTGG, in the pLenti6/V5-D-TOPO® vector.

The sequence of interest should include a Kozak translation initiation sequence with an ATG initiation codon for proper initiation of translation (Kozak, 1987; Kozak, 1991; Kozak, 1990). An example of a Kozak consensus sequence is (G/A)NNATGG. Other sequences are possible, but the G or A at position −3 and the G at position +4 are the most critical for function (shown in bold). The ATG initiation codon is underlined.

Below is the DNA sequence of the N-terminus of a theoretical protein and the proposed sequence for a forward PCR primer. The ATG initiation codon is underlined.

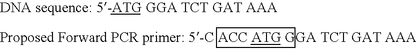

If the forward PCR primer is designed as above, then the primer includes the 4 nucleotides, CACC, required for directional cloning, and the ATG initiation codon falls within the context of a Kozak sequence (see boxed sequence), allowing proper translation initiation of the PCR product in mammalian cells. The first three base pairs of the PCR product following the 5′CACC overhang will constitute a functional codon.

When designing a reverse PCR primer, consider the points below. Refer to FIG. 48 for a diagram of the TOPO® Cloning site for pLenti6/V5-D-TOPO®. To ensure that the PCR product clones directionally with high efficiency, the reverse PCR primer should not be complementary to the overhang sequence GTGG at the 5′ end. A one base pair mismatch can reduce the directional cloning efficiency from 90% to 50%, increasing the likelihood of the PCR product cloning in the opposite orientation (see below). Evidence of PCR products cloning in the opposite orientation from a two base pair mismatch has not been observed.

To fuse a PCR product in frame with the C-terminal tag containing the V5 epitope, the reverse PCR primer can be designed to remove the native stop codon in the gene of interest (see below). To produce a native C-terminal on an expressed polypeptide, include the native sequence containing the stop codon in the reverse primer or make sure the stop codon is upstream from the reverse PCR primer binding site.

First Example of Reverse Primer Design. Below is the sequence of the C-terminus of a theoretical protein. The stop codon is underlined.

DNA sequence:
AAG TCG GAG CAC TCG ACG ACG GTG TAG-3′

To fuse the protein in frame with the C-terminal tag in pLenti6/V5-D-TOPO®, design the reverse PCR primer to start with the codon just up-stream of the stop codon, but the last two codons contain GTGG (underlined below), which is identical to the 4 bp overhang sequence. As a result, the reverse primer will be complementary to the 4 bp overhang sequence, increasing the probability that the PCR product will clone in the opposite orientation. This situation should be avoided.

DNA sequence:
AAG TCG GAG CAC TCG ACG ACG GTG TAG-3′
Proposed Reverse PCR primer sequence:
TG AGC TGC TGC CAC AAA-5′

Another solution is to design the reverse primer so that it hybridizes just down-stream of the stop codon, but still includes the C-terminus of the ORF. Note that the stop codon will need to be replaced by a codon for an innocuous amino acid such as glycine, alanine, or lysine.

Second Example of Reverse Primer Design

Below is the sequence for the C-terminus of a theoretical protein. The stop codon is underlined.

. . . GCG GTT AAG TCG GAG CAC TCG ACG ACT GCA TAG-3′

To fuse the ORF in frame with the C-terminal tag in pLenti6/V5-D-TOPO®, remove the stop codon by starting with nucleotides homologous to the last codon (TGC) and continue upstream. The reverse primer will be:

5′-TGC AGT CGT CGA GTG CTC CGA CTT-3′

This will amplify the C-terminus without the stop codon and allow the ORF to be joined in frame with the C-terminal tag. To avoid joining the ORF in frame with a C-terminal tag, design the reverse primer to include the stop codon.

5′-CTA TGC AGT CGT CGA GTG CTC CGA CTT-3′

pLenti6/V5-D-TOPO® accepts blunt-end PCR products. Do not add 5′ phosphates to primers for PCR. This will prevent ligation into the pLenti6/V5-D-TOPO® vector. It is recommended that oligonucleotides be gel-purified, especially if they are long (>30 nucleotides). Note that pLenti6/V5-D-TOPO® is supplied linearized with both ends adapted with topoisomerase I (see FIG. 47). The sequence of pLenti6/V5-D-TOPO™ is provided as Table 18.

Once a PCR strategy has been decided upon and primers synthesized, a blunt-end PCR product can be produced using any thermostable, proof-reading polymerase including, but not limited to, ThermalAce™, Platinum® Pfx, Pfu, or Vent® for PCR.

Follow the manufacturer's instructions and recommendations to produce blunt-end PCR products. It is important to optimize PCR conditions to produce a single, discrete PCR product. PCR fragments may be gel purified using standard techniques.

It is recommended that a 7 to 30 minute final extension be used in the PCR reaction to ensure that all PCR products are completely extended.

After the PCR reaction, the PCR product should be checked by removing 5 to 10 μl from each PCR reaction and using agarose gel electrophoresis to verify the quality and quantity of the PCR product. Check for a single, discrete band of the correct size. If there is not a single, discrete band, follow the manufacturer's recommendations for optimizing PCR with the polymerase of choice. Alternatively, gel-purify the desired product.

Estimate the concentration of the PCR product. A 5:1 molar ratio of PCR product:TOPO® vector is recommended to obtain the highest TOPO® Cloning efficiency (e.g. use 5-10 ng of a 1 kb PCR product or 10-20 ng of a 2 kb PCR product in a TOPO® Cloning reaction). Adjust the concentration of the PCR product as necessary before proceeding to TOPO® Cloning. If ThermalAce™ polymerase is used to produce blunt-end PCR product, note that ThermalAce™ can generate higher yields than other proofreading polymerases. When generating PCR products in the 0.5 to 1.0 kb range, generally the PCR reaction can be diluted 1:5 in 1× ThermalAce™ buffer before performing the TOPO® Cloning reaction. For PCR products larger than 1.0 kb, dilution may not be required.

Including salt (250 mM NaCl, 10 mM MgCl₂) in the TOPO® Cloning reaction may result in an increase in the number of transformants. Therefore, it is recommended that salt be added to the TOPO® Cloning reaction. A stock salt solution is provided in the kit for this purpose. Note that the amount of salt added to the TOPO® Cloning reaction varies depending on whether chemically competent cells (provided) or electrocompetent cells are to be transformed. For this reason two different TOPO® Cloning reactions are provided to obtain the best possible results.

Transforming Chemically Competent E. coli. For TOPO® Cloning and transformation into chemically competent E. coli, adding sodium chloride and magnesium chloride to a final concentration of 250 mM NaCl, 10 mM MgCl₂ in the TOPO® Cloning reaction increases the number of colonies over time. A Salt Solution (1.2 M NaCl, 0.06 M MgCl₂) is provided to adjust the TOPO® Cloning reaction to the recommended concentration of NaCl and MgCl₂.

Transforming Electrocompetent E. coli. For transformation of electrocompetent E. coli, the amount of salt in the TOPO® Cloning reaction should be reduced (e.g., to 50 mM NaCl, 2.5 mM MgCl₂) to prevent arcing. Dilute the Salt Solution 4-fold with water to prepare a 300 mM NaCl, 15 mM MgCl₂ solution for convenient addition to the TOPO® Cloning reaction (see below).

Setting Up the TOPO® Cloning Reaction. The table below describes how to set up a TOPO® Cloning reaction (6 μl) for eventual transformation into either chemically competent One Shot® TOP10 E. coli (provided) or electrocompetent E. coli. Additional information on optimizing the TOPO® Cloning reaction can be found herein. If the PCR product was generated using ThermalAce™ polymerase, note that it may be necessary to dilute the PCR reaction before proceeding. The blue color of the TOPO® vector solution is normal and is used to visualize the solution.

| | Chemically Competent | | Electrocompetent | |
|---|---|---|---|---|
| Reagents* | E. coli | | E. coli | |
| Fresh PCR product | 0.5 to 4 | μl | 0.5 to 4 | μl |
| Salt Solution | 1 | μl | — | |
| Sterile Water | add to a final volume of | | add to a final volume of | |
| | 5 | μl | 5 | μl |
| TOPO ® vector | 1 | μl | 1 | μl |

*Store all reagents at −20° C. when finished. Salt solution and water can be stored at room temperature or +4° C.

Performing the TOPO® Cloning Reaction. Mix reaction gently and incubate for 5 minutes at room temperature (22-23° C.). For most applications, 5 minutes will yield plenty of colonies for analysis. Depending on needs, the length of the TOPO® Cloning reaction can be varied from 30 seconds to 30 minutes. For routine subcloning of PCR products, 30 seconds may be sufficient. For large PCR products (>1 kb) or TOPO® Cloning a pool of PCR products, increasing the reaction time may yield more colonies.

Place the reaction on ice and transform suitable host cells using standard protocols. The TOPO® Cloning reaction can be stored at −20° C. overnight.

Transforming One Shot® TOP10 Competent E. coli. Once the TOPO® Cloning reaction has been performed, the pLenti6/V5-D-TOPO® construct is transformed into competent E. coli. One Shot® TOP10 Chemically Competent E. coli (Invitrogen Corporation, Carlsbad, Calif.) are included with the kit to facilitate transformation, however, electrocompetent cells may also be used. Protocols to transform chemically competent or electrocompetent E. coli are known to those skilled in the art. pLenti6/V5-D-TOPO® contains the ampicillin and blasticidin resistance genes for selection of transformants. Unwanted recombination (less than 5%) between the 5′ and 3′ LTRs has been observed when transformants are selected on LB agar plates containing ampicillin. These events occur less frequently when transformants are selected on LB agar plates containing ampicillin and blasticidin. Transformants should be selected on LB agar plates containing 50-100 μg/ml ampicillin AND 50 μg/ml blasticidin. Note that transformed E. coli grow more slowly in LB media containing ampicillin and blasticidin, and may require slightly longer incubation times to obtain visible colonies.

Transformants that contain a recombined plasmid generally give rise to larger colonies than those containing an intact plasmid. There is no blue-white screening for the presence of inserts. Most transformants will contain recombinant plasmid with the PCR product of interest cloned in the correct orientation. Sequencing primers are included in the kit to sequence across an insert in the multiple cloning site to confirm orientation and reading frame.

Addition of the Dilute Salt Solution to the TOPO® Cloning Reaction brings the final concentration of NaCl and MgCl₂ in the reaction to 50 mM and 2.5 mM, respectively. To prevent arcing of samples during electroporation, the volume of cells should be between 50 and 80 μl (0.1 cm cuvettes) or 100 to 200 μl (0.2 cm cuvettes). If arcing during transformation is seen, try reducing the voltage normally used to charge the electroporator by 10%, reducing the pulse length by reducing the load resistance to 100 ohms, and/or ethanol precipitating the TOPO® Cloning reaction and resuspending in water prior to electroporation.

After transformation and plating, pick 5 colonies and culture them overnight in LB or SOB medium containing 50-100 μg/ml ampicillin. Addition of blasticidin is not required. Isolate plasmid DNA using a method of choice. If ultra-pure plasmid DNA is need for automated or manual sequencing, the S.N.A.P.™ MidiPrep Kit (Invitrogen Corporation, Carlsbad, Calif. Catalog no. K1910-01) may be used. Analyze the plasmids by restriction analysis to confirm the presence and correct orientation of the insert. Use a restriction enzyme or a combination of enzymes that cut once in the vector and once in the insert.

Sequencing. The construct may be sequenced to confirm that the sequence of interest is cloned in the correct orientation and in frame with the V5 epitope. The CMV Forward and V5(C-term) Reverse primers are included in the kit and can be used to sequence the insert.

The sequence for pLenti6/V5-D-TOPO® shown in Table 18 includes the overhang sequence (GTGG) hybridized to CACC.

Analyzing Transformants by PCR. Transformants can be analyzed using PCR. For PCR primers, use a combination of the CMV Forward primer or the V5(C-term) Reverse primer and a primer that hybridizes within the insert. Appropriate amplification conditions can be determined by one skilled in the art. Results from the PCR reaction may be verified by conducting restriction analysis in parallel. Artifacts may be obtained in the PCR reaction because of mispriming or contaminating template.

If transformants or the correct insert are not obtained, perform the control reactions described below.

Once the correct clone has been identified, a glycerol stock of bacteria containing the plasmid may be prepared for long term storage. Also, a stock of plasmid DNA can be prepared and stored at −20° C.

Once a host cell containing a pLenti6/V5-D-TOPO® expression plasmid has been prepared, maintain and propagate the plasmid in LB medium containing 50-100 μg/ml ampicillin. Addition of blasticidin is not required.

Optimizing the TOPO® Cloning Reaction. The high efficiency of TOPO® Cloning allows the cloning process to be streamlined. To speed up the process of cloning PCR products, the TOPO® Cloning reaction can be incubated for only 30 seconds instead of 5 minutes. Fewer transformants may be obtained; however, because of the high efficiency of TOPO® Cloning, most of the transformants will contain the insert. After adding 2 μl of the TOPO® Cloning reaction to chemically competent cells, incubate on ice for only 5 minutes. Increasing the incubation time to 30 minutes does not significantly improve transformation efficiency.

When TOPO® Cloning large PCR products, toxic genes, or cloning a pool of PCR products, more transformants may be needed to obtain the desired clones. To increase the number of colonies incubate the salt-supplemented TOPO® Cloning reaction for 20 to 30 minutes instead of 5 minutes. Increasing the incubation time of the salt-supplemented TOPO® Cloning reaction allows more molecules to ligate and may increase the transformation efficiency. Addition of salt appears to prevent topoisomerase I from rebinding and nicking the DNA after it has ligated the PCR product and dissociated from the DNA.

To clone dilute PCR products, increase the amount of the PCR product, incubate the TOPO® Cloning reaction for 20 to 30 minutes, and/or concentrate the PCR product.

Once the sequence of interest has been TOPO® Cloned into pLenti6/V5-D-TOPO®, the ViraPower™ Lentiviral Expression System from Invitrogen Corporation, Carlsbad, Calif. can be used to produce a viral stock, which may then be used to transduce a mammalian cell line of choice to express the recombinant protein (as described above).

### Example 13

### Growth and Maintenance of the 293FT Cell Line

The 293FT cell line may be transported using any technique known to those skilled in the art, for example, by freezing the cells and transporting them on dry ice. For long term storage, the cells may be stored in liquid nitrogen. The 293FT cell line is supplied as one vial containing 3×10⁶ frozen cells in 1 ml of Freezing Medium.

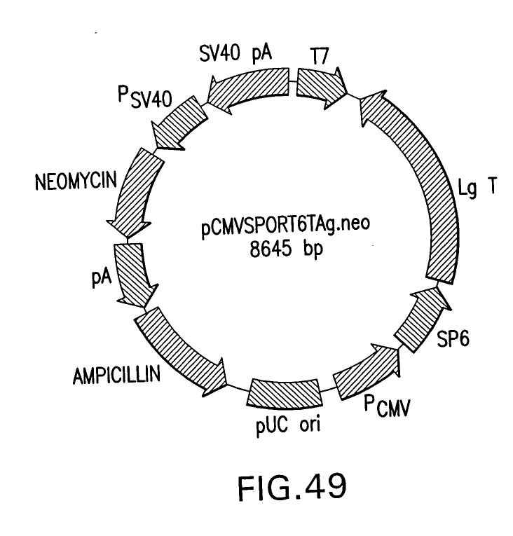

The 293FT cell line is genetically modified and carries the pUC-derived plasmid, pCMVSPORT6TAg.neo. A map of the vector is provided as FIG. 49. The pCMVSPORT6TAg.neo plasmid is derived from pCMVSPORT6, which has been modified to include the neomycin resistance gene for stable selection in mammalian cells (Southern and Berg, 1982, J. Molec. Appl. Gen. 1, 327-339). Expression of the neomycin resistance gene is controlled by the SV40 early enhancer/promoter from which the SV40 origin of replication has been removed. The plasmid also contains the gene encoding the SV40 large T antigen to facilitate optimal virus production (e.g. Invitrogen's ViraPower™ Lentiviral Expression System) and to permit episomal replication of plasmids containing the SV40 early promoter and origin. Expression of the SV40 large T antigen is controlled by the human cytomegalovirus (CMV) promoter.

The 293FT cell line is derived from the 293F cell line (see below) and stably expresses the SV40 large T antigen from the pCMVSPORT6TAg.neo plasmid. Expression of the SV40 large T antigen is controlled by the human cytomegalovirus (CMV) promoter and is high-level and constitutive. For more information about pCMVSPORT6TAg.neo, see below.

Studies have demonstrated maximal virus production in human 293 cells expressing SV40 large T antigen (Naldini et al., 1996), making the 293FT cell line a particularly suitable host for generating lentiviral constructs using the ViraPower™ Lentiviral Expression System available from Invitrogen (Catalog nos. K4950-00 and K4960-00).

The 293 cell line is a permanent line established from primary embryonal human kidney transformed with sheared human adenovirus type 5 DNA (Graham et al., 1977; Harrison et al., 1977, Virology 77, 319-329). The E1A adenovirus gene is expressed in these cells and participates in transactivation of some viral promoters, allowing these cells to produce very high levels of protein. The 293-F cell line available from Invitrogen Corporation, Carlsbad, Calif. (Catalog no. 11625) is a fast-growing variant of the 293 cell line, and was originally obtained from Robert Horlick at Pharmacopeia.

Antibiotic Resistance. 293FT cells stably express the neomycin resistance gene from pCMVSPORT6TAg.neo and should be maintained in medium containing Geneticin® at the concentration listed below. Expression of the neomycin resistance gene in 293FT cells is controlled by the SV40 enhancer/promoter. Geneticin® is available separately from Invitrogen Corporation, Carlsbad, Calif. (catalog number 11811-023).

Media for 293FT Cells. It is recommended that 293FT cells be grown in complete medium (D-MEM (high glucose), 10% fetal bovine serum (FBS), 2 mM L-glutamine, 1% Pen-Strep (optional)). For selection 500 μg/ml Geneticin® should be included. For freezing, 90% complete medium and 10% DMSO should be used. FBS does not need to be heat-inactivated for use with the 293FT cell line. 293FT cells should be maintained in medium containing Geneticina at the concentration listed above. If cells are split at a 1:5 to 1:10 dilution, they will generally reach 80-90% confluence in 3-4 days.

Follow the general guidelines below to grow and maintain 293FT cells. All solutions and equipment that come in contact with the cells must be sterile. Always use proper sterile technique and work in a laminar flow hood. Before starting experiments, be sure to have cells established and also have some frozen stocks on hand. Early-passage cells are recommended for experiments. Upon receipt of the cells from Invitrogen Corporation, Carlsbad, Calif., grow and freeze multiple vials of the 293FT cell line to ensure that an adequate supply of early-passage cells is available.

For general maintenance of cells, pass 293FT cells when they are 80-90% confluent (generally every 3-4 days). Avoid overgrowing cells before passaging.

Use trypan blue exclusion to determine cell viability. Log phase cultures should be >90% viable.

When thawing or subculturing cells, transfer cells into pre-warmed medium.

Cells should be at the appropriate confluence and at greater than 90% viability prior to transfection.

As with other human cell lines, when working with 293FT cells, handle as potentially biohazardous material under at least Biosafety Level 2 (BL-2) containment.

The following protocol is designed to thaw 293FT cells to initiate cell culture. The 293FT cell line is supplied in a vial containing 3×10⁶ cells in 1 ml of Freezing Medium.

Remove the vial of cells from the liquid nitrogen and thaw quickly in a 37° C. water bath. Just before the cells are completely thawed, decontaminate the outside of the vial with 70% ethanol, and transfer the cells to a T-75 flask containing 12 ml of complete medium without Geneticin®. Incubate the flask at 37° C. for 2-4 hours to allow the cells to attach to the bottom of the flask. Aspirate off the medium and replace with 12 ml of fresh, complete medium without Geneticin®. Incubate cells overnight at 37° C. The next day, aspirate off the medium and replace with fresh, complete medium containing Geneticin® at the recommended concentration listed above. Incubate the cells and check them daily until the cells are 80-90% confluent (2-7 days).

Passaging Cells. When cells are ˜80-90% confluent, remove all medium from the flask. Wash cells once with 10 ml PBS to remove excess medium and serum. Serum contains inhibitors of trypsin. Add 5 ml of trypsin/versene (EDTA) solution to the monolayer and incubate 1 to 5 minutes at room temperature until cells detach. Check the cells under a microscope and confirm that most of the cells have detached. If cells are still attached, incubate a little longer until most of the cells have detached. Add 5 ml of complete medium to stop trypsinization. Briefly pipette the solution up and down to break up clumps of cells.

To maintain cells in 75 cm² flasks, transfer 1 ml of the 10 ml cell suspension from above to a new 75 cm² flask and add 15 ml fresh, complete medium containing Geneticin®. To have the cells reach confluency sooner, split the cells at a lower dilution (i.e. 1:4).

To expand cells into 175 cm² flasks, add 28 ml of fresh, complete medium containing Geneticin® to each of three 175 cm² flasks, then transfer 2 ml of the cell suspension to each flask to obtain a total volume of 30 ml.

Incubate flasks in a humidified, 37° C., 5% CO₂ incubator.

Passage the cells as necessary to maintain or expand cells.

Freezing Cells. When freezing the 293FT cell line, it is recommended that the cells be frozen at a density of at least 3×10⁶ viable cells/ml. Use a freezing medium composed of 90% complete medium and 10% DMSO. Complete medium is medium containing serum.

Preparing Freezing Medium. Freezing medium should be prepared immediately before use. In a sterile, conical centrifuge tube, mix together 0.9 ml of fresh complete medium and 0.1 ml of DMSO for every 1 ml needed. Place the tube on ice until use. Discard any remaining freezing medium after use.

Freezing the Cells. Before starting, label cryovials and prepare freezing medium (see above). Keep the freezing medium on ice. To collect cells, count the cells prepared by trypsinization as described in Passaging the Cells above. Pellet cells at 250×g for 5 minutes in a table top centrifuge at room temperature and carefully aspirate off the medium. Resuspend the cells at a density of at least 3×10⁶ cells/ml in chilled freezing medium. Place vials in a microcentrifuge rack and aliquot 1 ml of the cell suspension into each cryovial. Freeze cells in an automated or manual, controlled-rate freezing apparatus following standard procedures. For ideal cryopreservation, the freezing rate should be a decrease of 1° C. per minute. Transfer vials to liquid nitrogen for long-term storage.

The viability and recovery of frozen cells may be checked 24 hours after storing cryovials in liquid nitrogen by following the procedure outlined in Thawing above.

Transfecting Cells. The 293FT cell line is generally amenable to transfection using standard methods including calcium phosphate precipitation (Chen and Okayama, 1987, Molec. Cell. Biol. 7, 2745-2752; Wigler et al., 1977, Cell 11, 223-232), lipid-mediated transfection (Felgner et al., 1989, Proc. West. Pharmacol. Soc. 32, 115-121; Felgner and Ringold, 1989, Nature 337, 387-388), and electroporation (Chu et al., 1987, Nucleic Acids Res. 15, 1311-1326; Shigekawa and Dower, 1988, BioTechniques 6, 742-751). Typically cationic lipid-based transfection reagents are used to transfect 293FT cells. Lipofectamine™ 2000 (Invitrogen Corporation, Carlsbad, Calif. catalog number 11668-027) is recommended, but other transfection reagents are suitable.

Transient Transfection. The 293FT cell line may be transiently transfected with any plasmid. Make sure that cells are healthy at the time of plating. Overgrowth of cells prior to passaging can compromise their transfection efficiency. On the day before transfection, plate cells such that they will be approximately 60% confluent at the time of transfection. If Lipofectamine™ 2000 is to be used as a transfection reagent, plate cells such that they will be 90-95% confluent at the time of transfection. Transfect the plasmid construct into the 293FT cell line using the method of choice (see above). After transfection, add fresh medium containing 500 μg/ml Geneticin® and allow the cells to recover for 24-48 hours before proceeding to assay for expression of the gene of interest.

Generating Stable Cell Lines. 293FT cells can be used as hosts to generate a stable cell line expressing a gene of interest from most plasmids.

Remember that the introduced plasmid must contain a selection marker other than neomycin resistance. Stable cell lines can then be generated by transfection and dual selection with Geneticin® and the appropriate selection agent.

Since 293FT cells stably express the SV40 large T antigen, generating stable cell lines with plasmids that contain the SV40 origin of replication is not recommended.

### Example 14

### Use of Suppressor tRNAs to Transiently Label Proteins of Interest

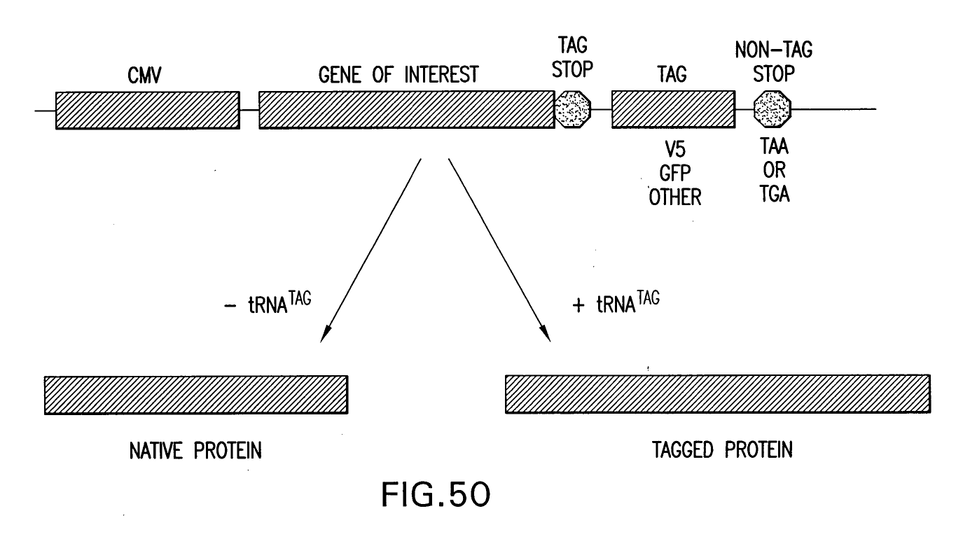

This example describes the use of mammalian suppressor tRNAs (e.g., tRNA^{ser}) that specifically recognize and decode one of the three stop codons: amber (TAG), opal (TGA) or ochre (TAA) as an amino acid (e.g., serine). Expression plasmids encoding a gene of interest with one of these stop codons will express a native protein under normal conditions (see FIG. 50). If the appropriate tRNA suppressor is supplied, that stop codon will be translated (e.g., as serine when tRNA^{ser} is used) and translation will continue through any downstream reading frame, creating a fusion protein consisting of the protein of interest with a specific C-terminal epitope tag (see FIG. 50). “Gene of interest” as used herein, refers to, for example, a nucleic acid sequence encoding a polypeptide, a protein, or an untranslated RNA, e.g., tRNA, all of which are encompassed by the term.

One non-limiting example of this stop suppression technology, termed Tag-On-Demand™ available from Invitrogen Corporation, Carlsbad, Calif., which allows expression of tagged or untagged proteins using a single gene expression vector. In this embodiment, recombinant adenovirus vectors carrying the amber (TAG) stop suppressor tRNA gene have been developed as well as optimized protocols for use in transiently tagging a protein of interest in mammalian cells. The specific embodiment described here is purified, titered recombinant adenovirus (Adeno-tRNA^{TAG}) and one new GATEWAY™ Destination vector (pcDNA6.2/GFP-DEST). Tag-On-Demand™ may be used with any gene of interest provided the stop codon is TAG. For example, additional Invitrogen mammalian expression vectors that are compatible with Tag-On-Demand™ are listed below.

The use of the pcDNA6.2/V5 and pcDNA6.2/GFP Destination vectors is recommended for use in Tag-On-Demand™ primarily due to the superiority of blasticidin as a selectable marker and the absence of the BGH polyA. In addition to the recommended vectors listed above, the following three Destination vectors have also been successfully used in Tag-On-Demand™ pcDNA3.2/V5-DEST, pcDNA-DEST40, and pcDNA-DEST47. The following Invitrogen vectors are all compatible with Tag-On-Demand™ and contain a non-TAG stop codon downstream of the C-terminal epitope tag, provided the gene of interest is cloned with TAG stop in frame with the C-terminal tag: pcDNA/V5His vector family; pEF/V5His vector family; pUbC/V5His vectors; pcDNA/mycHis vector family; pEF/mycHis vector family; pcDNA3.1/CT-GFP vectors; pcDNA4/TO/mycHis vectors; pGene/V5His vectors; pIND/V5His vectors; pcDNA5/FRT/V5His; and pEF5/FRT vectors.

Materials and Methods

Vector construction. (a) pUC12-tRNA^{TAG}: Three suppressor tRNA vectors were received from Dr. Uttam RajBhandary of Massachusetts Institute of Technology. Each suppressor tRNA vector, designated pUCtS Su+ amber, opal, and ochre, is identical except for the stop anticodon (Capone et. al. 1985, EMBO, 4(1):213-221). For convenience, the pUCtS Su+ amber vector is now referred to as pUC12-tRNA^{TAG}. To create a tetracycline-regulated version, referred to herein as pUC12-TO-tRNA^{TAG}, two tetracycline operators (tetO₂) were cloned into the SnaBI site in pUC 12-tRNA^{TAG} using the following annealed oligonucleotides:

tetO₂ Forward primer

5′ GACTCGAGTCTCCCTATCAGTGATAGAGATCTCGAGGTC 3′ and

tetO₂ Reverse primer

5′ GACCTCGAGATCTCTATCACTGATAGGGAGACTCGAGTC3′.

In italics is a unique BglII site that was introduced with the oligonucleotide. The underlined sequences are XhoI sites. All tRNA constructs were sequence verified.

(b) pcDNA6.2/GFP-DEST: pcDNA6.2/V5-DEST was digested with ApaI and PmeI to remove the V5 tag. pcDNA3.1/lacZ-stop^{TAG}-GFP was also digested with ApaI and PmeI to isolate the GFP fragment. The GFP fusion tag was ligated to the pcDNA6.2 DEST vector (Invitrogen Corporation, Carlsbad, Calif. catalog # 12489-027) and transformed into DB3.1 cells. Colonies were grown on LB-Amp plates. A clone was selected that resulted in correct band fragments when digested with NdeI and then sequence confirmed.

(c) pENTR CAT^{TAA,TAG,TGA} The GATEWAY™ CAT entry clones were PCR amplified followed by TOPO cloning (Invitrogen Corporation, Carlsbad, Calif. product manual #25-0434) into pENTR dT. Informration for both vectors may be obtained by contacting Invitrogen Corporation, Carlsbad, Calif. The primer sequences used were

| | |
|---|---|
| Forward primer: | 5′ CACCATGGAGAAAAAAATCACTGG 3′ |
| Reverse primer: | 5′ CTGCTACGCCCCGCCCTGC 3′. |

The underlined sequence varied depending on which stop codon was required. Plasmid constructs were sequence verified.

(d) pcDNA3.2/V5-GW/CAT^{TAA, TAG, TGA}: pcDNA3.2/V5-DEST and pENTR CAT with each of the stops was recombined using LR clonase to generate the plasmids pcDNA3.2/V5-GW/CAT^{TAA, TAG, TGA}. Clones were identified as correct by restriction enzyme digests and sequence confirmed.

(e) pcDNA6.2/GFP-GW/CAT^{TAA, TAG, TGA}: pcDNA6.2/GFP-DEST and pENTR CAT with each of the stops was recombined using LR clonase to generate the plasmids pcDNA6.2/GFP-GW/CAT^{TAA, TAG, TGA}. Clones were identified as correct by restriction enzyme digests and sequence confirmed.

(f) pENTR p48^{TAG}: This GATEWAY™ Entry clone was obtained from the Ultimate™ ORFeome Collection (Invitrogen Corporation, Carlsbad, Calif.) and is referred to by several names: HS8-E6 (internal Invitrogen designation), BC000141 (GenBank Accession number), or ORF 12 (used for convenience). This ORF is referred to as p48 and is a human c-myc variant (see Results section). Information for this clone may be obtained by contacting Invitrogen Corporation, Carlsbad, Calif. or GenBank.

(g) pcDNA6.2/GFP-GW/p48^{TAG}: pcDNA6.2/GFP-DEST and pENTR p48^{TAG} were recombined with LR clonase to generate pcDNA6.2/GFP-GW/p48^{TAG}. The recombination reaction was transformed into TOP10 cells (Invitrogen Corporation, Carlsbad, Calif., catalog #C4040-10) and plated on LB Ampicillin plates. Colonies were picked and clones were identified as correct by restriction enzyme digests and functional suppression.

(h) pcDNA6.2/V5-GW/p48^{TAG}: pcDNA6.2/V5-DEST and pENTR p48^{TAG} were recombined with LR clonase to generate the plasmid pcDNA6.2/V5-GW/p48^{TAG}. The recombination reaction was transformed into TOP10 cells and plated on LB Ampicillin plates. Colonies were picked and clones were identified as correct by restriction enzyme digests and functional suppression.

(i) pENTR-TO-tRNA^{TAG}: pENTR1A (Invitrogen Corporation, Carlsbad, Calif.) and pUC12-TO-tRNA^{TAG} (described in (a) above) were digested with SalI and EcoRI. Following digests, the appropriate bands were gel purified and ligated. Ligations were transformed into TOP10 cells and plated on LB-Kanamycin plates. Clone 1 was selected following SalI and EcoRI diagnostic digests.

(j) pENTR-tRNA8^{TAG}: Primers were created to PCR amplify the tRNA gene from pUC12 TO tRNA^{TAG} with EcoRI and XbaI sequences at the 5′ end, and SpeI and HindIII at the 3′ end. The primer sequences were:

Forward primer:

5° CACCGAATTCTCTAGAGATGTCTGTGAAAAGAAACAT 3′ and

Reverse primer:

5′ ATATAAGCTTACTAGTCCGGATTTCCTCTACCCGAGA 3′.

The tRNA PCR product was gel purified, TOPO cloned into pENTR dT, and transformed into TOP10 cells. Colonies were selected on LB Kanamycin plates. Upon confirmation of proper insertion, two separate digests were conducted. The first digest with EcoRI and XbaI opened the pENTR-tRNA^{TAG}. The second digest with EcoRI and SpeI excised the tRNA gene. Correct fragments were gel purified, the two fragments were ligated, as XbaI and SpeI have complimentary ends, thus creating a dimer of tRNA. With confirmation of proper insertion, the same two previous digests were repeated with the dimer plasmid, fragments gel purified, ligations performed creating a tetramer. A final two digests, as previously described, were repeated on the tetramer, fragments gel purified, ligations performed creating an octamer tRNA in the pENTR backbone. (Buvoli et al., Mol. Cell. Biol. 20:3116-3124 (2000), Suppression of Nonsense Mutations in Cell Culture and Mice by Multimerized Suppressor tRNA Genes).

Adenovirus tRNA

Adenovirus carrying the suppressor tRNA^{TAG} was created using a GATEWAY™ LxR reaction. pAd/PL-DEST vector (Table 10, FIG. 9) was recombined with either pENTR-tRNA^{TAG} or pENTR-tRNA8^{TAG} to create pAd-tRNA^{TAG} (Table 8) or pAd-tRNA8^{TAG} expression vectors, respectively. These vectors were subsequently cut with PacI and transfected into TREx 293 (Invitrogen Corporation, Carlsbad, Calif., catalog #R710-07) cells to produce the initial stocks of recombinant adenovirus. Subsequent virus amplification and titering was performed in 293A cells as previously described in Example 4.

Adenovirus Production and Purification

Ten T-175 flasks of 293A cells were plated in 25 ml of complete medium per flask (DMEM/10% FBS/L-Glutamine/non-essential amino acids/penicillin/streptomycin). On the day of infection, the cells were 80-90% confluent. The old media was removed and replaced with 25 ml of complete media containing sufficient virus for an MOI of 5 viruses per cell. Cultures were incubated overnight at 37° C. The next day, the media was replaced with 25 ml fresh media and the cells were incubated for 2-3 days until >80% cytopathic effect (CPE) was observed. CPE is obvious: the cells swell, round-up, and begin to detach from the plate. At this point, the cells were gently dislodged using a 50 ml pipette and pooled in 250 ml sterile conical bottles. Cultures were centrifuged at 1000 rpm for 10 minutes. The supernatant was discarded, the cell pellet was dissolved in 5 ml of PBS and transferred to a 15 ml polypropylene tube. Cells were lysed to release virus by three freeze/thaws (−80° C. to 37° C.). Care was taken not to leave sample at 37° C. any longer than necessary to melt it, as virus degradation is accelerated at 37° C. After the freeze/thaw cycles, 150 μl was removed for the wild type assay (see below). The lysates were then treated with DOC (deoxycholate, sodium salt, Sigma-Aldrich, St. Louis, Mo. catalogue #D 6750) to increase the virus yields. A stock of 10% DOC was prepared in water (heat was required to get it all into solution) and DOC was added to the adenovirus lysate to a final concentration of 0.2%. The lysates were incubated at room temperature for 30 minutes on a rotating platform. Insoluble materials were eliminated by centrifugation (3000 rpm for 15 minutes in table top centrifuge), and the crude high titer viral supernatant (CHT) was transferred to a fresh tube. MgCl₂ was added to 5 mM final and virus was stored at −80° C. or cesium chloride purified (see below).

Cesium chloride step-gradient ultracentrifugation purifies and concentrates the recombinant adenovirus by eliminating cellular contaminants in order to achieve optimum efficiency and minimal toxicity of adenovirus gene delivery. Two cesium chloride (Molecular Biology grade CsCl, Sigma-Aldrich, St. Louis, Mo., catalog #C3032) solutions were prepared with the following densities: 1.4 g/ml and 1.25 g/ml in 10 mM Tris (pH 8.0). [1.4 g/ml=38.83 g CsCl+61 ml 10 mM Tris and 1.25 g/ml=26.99 g CsCl+73 ml 10 mM Tris] These weight/volume densities were verified by weighing 1 ml of each solution. Density was adjusted by adding more cesium chloride or 10 mM Tris as needed to achieve correct density. Each solution was filter sterilized and stored at room temperature.

To prepare the step-gradient, 2.5 ml of 1.25 g/ml CsCl solution was placed in one ultracentrifuge tube (SW41 Beckman centrifuge tubes, thin wall polyallomer 14×89, part#331372) and then carefully underlayed with 2.5 ml of 1.4 g/ml CsCl solution. A long glass Pasteur pipette was used for underlaying. Next, the step-gradient was gently overlaid with the 5 ml of crude high titer viral lysate and carefully move into Beckman SW41 centrifuge rotor. Samples were spun at 35,000 rpm for one hour and ten minutes at 20° C. After ultracentrifugation, cellular lipids and cytoplasmic debris remained at the top of the tube, and the cloudy adenovirus band migrated near the interface of the two CsCl layers. The virus band was very obvious to the naked eye. The tube was clamped to a ring stand and the sides of the tube were wiped with 70% ethanol. The virus band was harvested using a 3 ml syringe fitted with a 20 or 21 gauge needle by side puncture. Virus was transferred to a 15 ml conical tube and the volume estimated. Glycerol was added to 10% final.

The cesium chloride in the recombinant adenoviral preparation was removed by four rounds of dialysis. For each ultracentrifuge tube four liters of dialysis buffer was required. The dialysis buffer consisted of 10 mM Tris (pH 7.5), 1 mM MgCl₂, 150 mM NaCl, and 10% glycerol final and was kept at 4° C. Buffer was prepared the day before dialysis and placed in the cold room with a stir bar overnight. The recovered viral band was dialyzed at 4° C. using. a Spectrum Specta/Por CE Float-A-Lyzer with a molecular weight cut-off (MWCO) of 300,000 Dalton (Fischer 3 ml size #08-700-51 or 5 ml size #08-700-64). The virus preparation was dialyzed four times. Each dialysis was conducted for one hour and in one liter, with constant gentle stirring. The final virus product was removed from Float-A-Lyzer with the plastic pipette provided and aliquotted into eppendorf tubes. Aliquots of virus were stored at −80° C. and multiple freeze/thaws were avoided.

Typical titers from a 10-flask cesium chloride preparation range from 7×10⁹ to 6.5×10¹⁰ pfu/ml. The volume of purified recombinant adenovirus obtained is typically around 1.0 ml, making the total virus yield from 10-flasks to be 7×10⁹ to 6.5×10¹⁰ pfu. This stock contains enough material for one 96-well plate using an MOI of 50.

Wild-Type Assay

The “supernatant rescue assay” is performed to detect wild-type adenovirus contamination in recombinant adenovirus stocks using standard procedures (Dion et al., J. Virol. Methods 56:99-107 (1996)).

Reporter Cell Line

pcDNA6/FRT/V5 was digested with PstI and PmeI to remove the nucleic acid sequence encoding the V5 tag. pcDNA3.1 lacZ stop^{TAG} GFP was digested with PstI and Pme I to isolate the fragment containing lacZ stop^{TAG} GFP. The above fragments were gel purified, ligated, and transformed into TOP10 cells. The resulting reporter plasmid, pcDNA6/FRT/lacZ-stop^{TAG}-GFP, was verified by diagnostic digests and sequencing. FlpIn CHO cells (Invitrogen Corporation, Carlsbad, Calif., catalog #R758-07) were co-transfected with the vector pcDNA6/FRT/lacZ stop^{TAG} GFP and pOG44 (invitrogen Corporation, Carlsbad, Calif., catalog #V6005-20) at a ratio of 1:10. Blasticidin selection was started 4 days post transfection at a concentration of 15 μg/ml. Selection was complete after 24 days.

Co-transfections

Six-well plates were seeded with cells one day prior to transfections. Cells tolerate transfections best if seeded at a density that allowed for greater than 90% confluency the day of co-transfection. Co-transfections were conducted for a minimum of 5 hours and up to overnight. The lipid complexes were then removed and replaced with fresh media. Co-transfections were optimized using 1.5 μg suppressor tRNA plasmid and 0.5 μg of the corresponding reporter vector combined in 250 μl of Opti-MEM® I Reduced Serum Medium (OPTI-MEM) at room temperature for 5-10 minutes. Six microliters of Lipofectamine™ 2000 was combined with 250 μl of OPTI-MEM and allowed to sit at room temperature for 5 minutes before combining with the DNA mixture. The DNA-lipid complex was allowed to form for 20 minutes at room temperature. Subsequently, the DNA-lipid complex was added to the cells in wells containing 2 ml of media. Suppression in a GFP fused expression vector could be observed the following day and up to 72 hours post transfection. Cells were typically lysed and harvested twenty-four hours post transfection with IGE PAL CA630 lysis buffer (Sigma-Aldrich, St. Louis, Mo., catalog #I-3021) or RIPA lysis buffer (10 mM Tris (8.0), 150 mM NaCl, 0.1% SDS, 1.0% NP-40 (or Triton X-100), 1.0% deoxycholate, 2 mM EDTA) with leupeptin, pepstatin, and PMSF.

Transductions

Cells were transduced with suppressor tRNA for a minimum of five hours to a maximum of overnight in a total of 1 ml media in a six well format. Upon completion of transduction the virus was removed and 2 ml of fresh media was added. The cells were then transfected overnight or the following day. Cells were typically lysed and harvested three days post transduction with IGE PAL CA630 lysis buffer or RIPA lysis buffer with leupeptin, pepstatin, and PMSF.

Westerns

Cell lysates were centrifuged at maximum speed for 1-2 minutes. Lysates were then transferred to new tubes and pellet discarded. A Bradford protein assay was conducted to determine the protein concentration of each lysate. For western blotting, 10-30 μg of protein was loaded on a gel. Determination of percent suppression was performed using 6% Tris Glycine gels for western blotting of β-galactosidase fusion proteins to maximize resolution of high molecular weight proteins. For CAT, GFP and V5 blots, 4-20% Tris Glycine gels were used. Proteins from gels were transferred to 0.45 μm nitrocellulose using western blotting technique. Various antibodies were used in detection of proteins: anti-βgal at 1:5000, anti-CAT at 1:5000, anti-GFP at 1:5000, anti-V5 at 1:5000. Western Breeze kits and antibodies from Invitrogen Corporation, Carlsbad, Calif. were used throughout.

QC Assay for Manufactured Virus

Virus produced in manufacturing should be screened for wild type virus, cesium chloride purified and plaque-assay titered. For the activity assay, COS-7 cells (ATCC #CRL-1651) were seeded at a density of 3×10⁵ cells in a 6 well format with 2 ml of DMEM containing 10% FBS, 1% L-glutamine, and 1% Pen/Strep. The following day, the media was aspirated and 1 ml was added back to the culture wells to be transduced. CsCl purified Ad-tRNA8^{TAG} was added to each well at an MOI of 0, 25, 50 and 100. The transductions were allowed to proceed for 5-6 hours at 37° C. Following the transduction period, the media containing the virus was removed and 2 ml of fresh media was added back to each well. The transduced cells were allowed a day to recover before transfection of reporter plasmid. For each transfected well, two micrograms of pcDNA6.2/GFP-GW/p48^{TAG} (or pcDNA3.1/lacZ-STOP^{TAG}-GFP) expression plasmid was diluted in 250 μl of OPTI-MEM and incubated at room temperature for 5 minutes. 6 μl of Lipofectamine™ 2000 was diluted in 250 μl of OPTI-MEM and incubated at room temperature for 5 minutes. The DNA and lipid dilutions may also be set-up in batch for the four wells to be transfected. The DNA and lipid were then combined and incubated at room temperature for 20 minutes to complex before adding to COS-7 cells previously transduced with Ad-tRNA8^{TAG}. The DNA-lipid complex remained on the cells between 5 to 18 hours before being removed and fresh media added to the cells. GFP fluorescence was observed on days 1-3 post transduction. The cells were lysed and harvested with ice cold 150-200 μl of RIPA lysis buffer (10 mM Tris (8.0), 150 mM NaCl, 0.1% SDS, 1.0% NP-40 (or Triton X-100), 1.0% deoxycholate, 2 mM EDTA) containing leupeptin, pepstatin, and PMSF on day 3 post transduction. The lysates were then centrifuged for 5 minutes at maximum speed (preferably at 4° C.). Lysates were transferred to new 1.5 ml eppendorf tubes and frozen at −80° C. if not used immediately for western blotting. Following western blotting for anti-myc (or anti-β-galactosidase, depending on the expression plasmid used) densitometry was performed on the Fujifilm LAS-1000 Densitometer using the software Image Reader LAS-1000 Lite v1.0 and imageGauge v.254. Percent suppression was calculated by dividing the density of the upper band by the total (lower plus upper band). For the purpose of this example, 50% suppression was the desired level of suppression.

Results and Discussion

All three possible human tRNA suppressors (TAG, TAA and TGA) were created by mutating the anticodon of the human tRNA serine gene (Capone et al., EMBO, 4:213-221 (1985)). This work was performed in the laboratory of Dr. RajBhandary, who also provided the pUC12-based vectors containing each of the three tRNA^{ser} suppressors. Bacterial tRNA suppressors had been identified many years previously, but the use of a mammalian tRNA suppressor allows stop suppression in mammalian cells without the need to co-express the cognate tRNA charging enzyme.

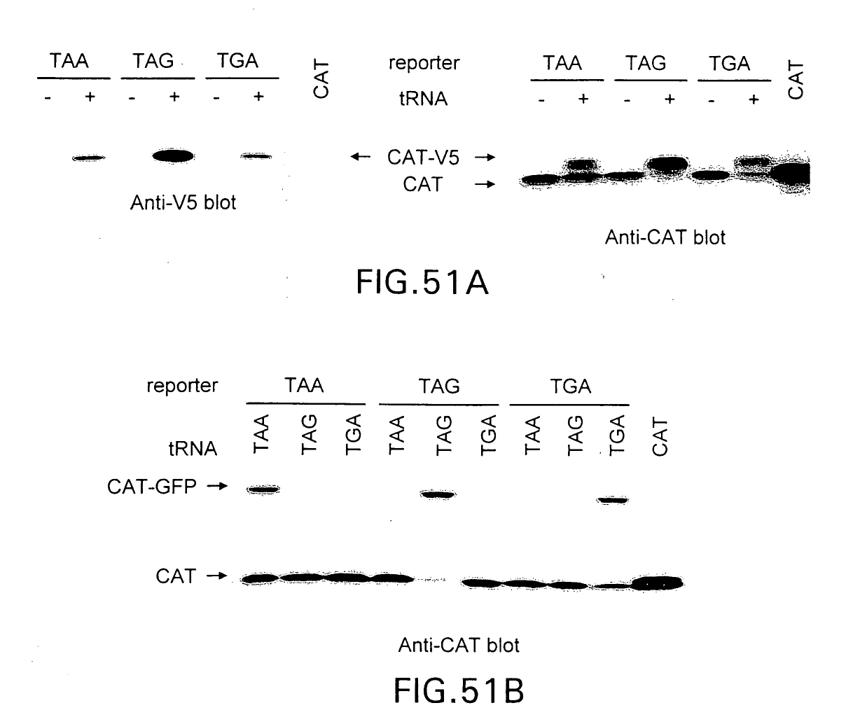

The efficiency of each tRNA suppressor was tested in several co-transfection experiments (FIGS. 51A-B). Three GATEWAY™ entry clones were created, with CAT as the gene of interest (GOI), followed by each of the three stop codons (pENTR-CAT^{TAA}, pENTR-CAT^{TAG} and PENTR-CAT^{TGA}). These entry clones were LR crossed into either pcDNA3.2/V5-DEST or pcDNA6.2/GFP-DEST, thus placing either V5 or GFP downstream (and in frame) of the native CAT ORF. These Destination vectors also have all three stop codons, in frame, downstream of the C-terminal tag. Having all three stop codons assures termination of translation after the tag, regardless of which tRNA suppressor is used.

V5 Epitope Tag-On-Demand™

CHO cells were co-transfected with one of these expression vectors: pcDNA3.2/V5-GW/CAT^{TAA}, -GW/CAT^{TAG} or -GW/CAT^{TGA} in the presence or absence of its cognate tRNA suppressor: pUC12-tRNA^{TAA}, pUC12-tRNA^{TAG} or pUC12-tRNA^{TGA} (FIG. 51A). Western blot analysis using antibodies against the V-5 epitope revealed easily detectable V5-epitope-tagged protein in the presence of tRNA suppressor (left panel), which was further illustrated by the “shift” up of CAT protein on the anti-CAT western blot (right panel). The efficiency of suppression can be calculated by using densitometry to scan the intensity of the shifted and un-shifted bands. In this experiment as well as others not described herein, the TAG stop suppressor was clearly superior to the other two, demonstrating a >70% conversion of native CAT to CAT-V5 in the presence of the suppressor (FIG. 51A, right panel, anti-CAT blot), as compared to only 44% and 53% for TAA and TGA, respectively.

GFP Tag-On-Demand™

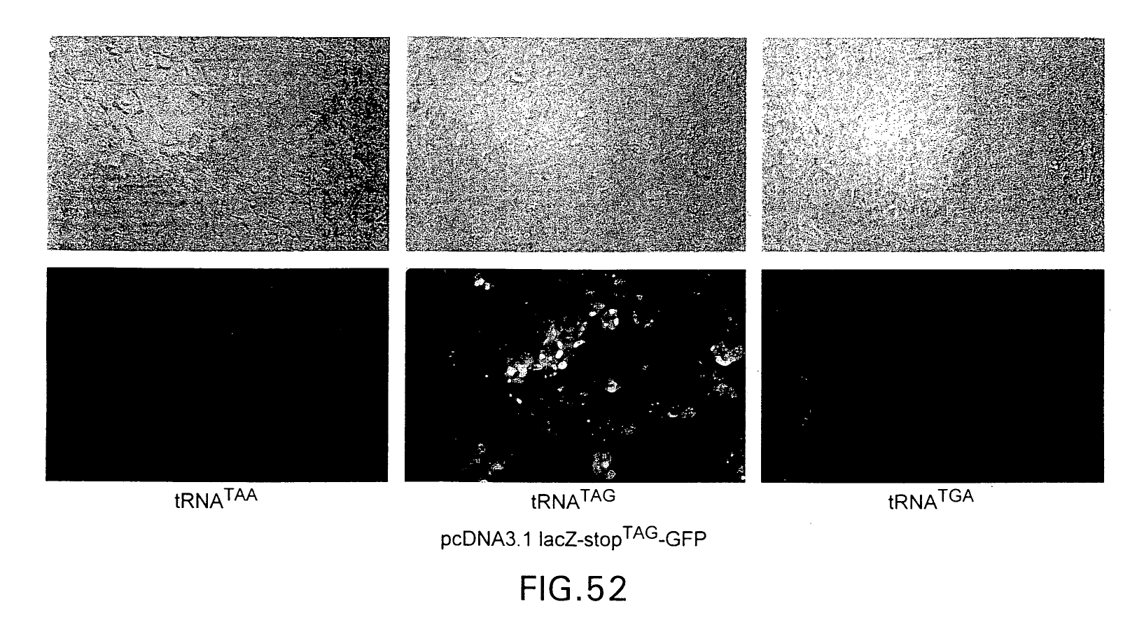

293FT cells (Invitrogen Corporation, Carlsbad, Calif., catalog #R700-07) were co transfected with one of the three expression vectors (pcDNA6.2/GFP-GW/CAT^{TAA}, -GW/CAT^{TAG} or -GW/CAT^{TGA}) and one of the pUC12-tRNA vectors (FIG. 51B). Anti-CAT western blotting showed a clear shift of native CAT up to CAT-GFP when the correct tRNA was supplied. Again, tRNA^{TAG} demonstrated superior stop suppression compared to the other two tRNA suppressors. It is also important to note that the stop suppression and protein tagging is very specific. In other words, when the incorrect tRNA suppressor is supplied, no stop suppression is observed and only native protein is expressed (for example, see TAA CAT reporter with tRNA^{TAG} or tRNA^{TGA}, FIG. 51B). The specificity of the suppression is further demonstrated with a different reporter vector, pcDNA3.1/lacZ-stop^{TAG}-GFP (FIG. 52). Only in the presence of the correct tRNA suppressor (pUC12-tRNA^{TAG}) was the β-galactosidase-GFP fusion protein expressed resulting in detectable glowing in the cells (center panels, FIG. 52). When either of the other suppressors is used (tRNA^{TAA} or tRNA^{TGA}), no suppression of the TAG stop occurs, no β-galactosidase-GFP is expressed and no glowing is observed in the transfected cells (left and right panels FIG. 52).

Adenovirus-tRNA Delivery

Since Tag-On-Demand™ is primarily designed for the transient tagging of proteins, an ideal delivery method of the suppressor gene to mammalian cells is using a recombinant adenovirus. Adenovirus has a very broad tropism for different mammalian cell types and transduction efficiencies can approach 100% (for review see Russell 2000, Update on adenovirus and its vectors. J. Gen. Virol., 81:2573-2604). Furthermore, since the virus does not stably integrate into the host genome, expression is transient. In actively dividing cells (24 hour doubling time), gene expression from adenoviral vectors is typically detected within 24 hours and persists for 7-8 days.

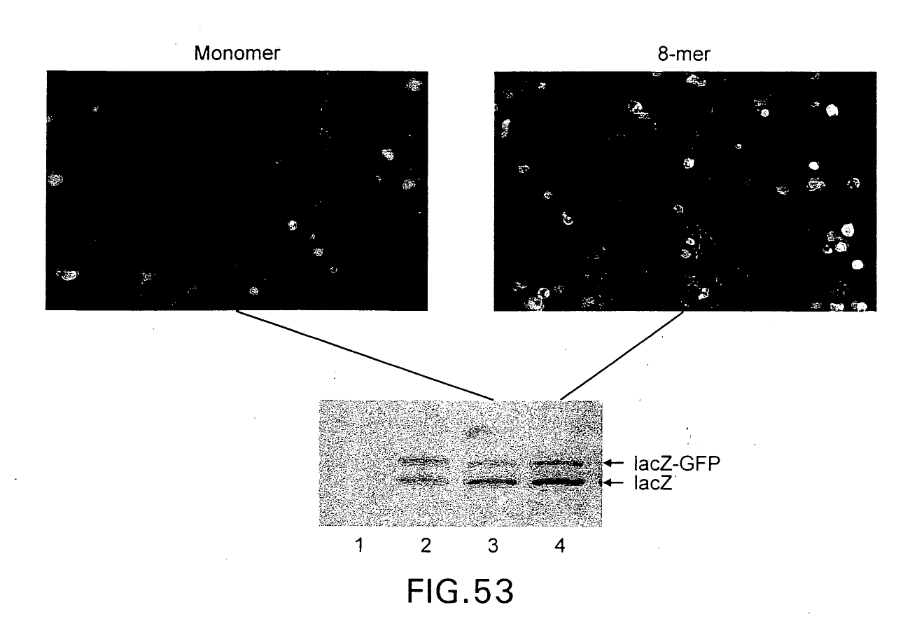

The tRNA^{TAG} gene was cloned into pENTR to create pENTR-tRNA^{TAG}, and this was used in a GATEWAY™ LR reaction with pAd/PL-DEST (Table 10, FIG. 9) to create pAd-tRNA^{TAG}. Several large-scale preparations of virus were performed and functional testing was done. Adenovirus proved to be a very efficient way of delivering the tRNA, however preliminary experiments required MOIs (multiplicity of infection) of several hundred to deliver biologically relevant amounts of the tRNA. The goal was to achieve at least 50% suppression using an MOI of 50 in COS cells transfected with one of the reporter genes. It is believed that the tRNAs must compete with endogenous protein “stop factors” occupying the stop codon, which may explain the more efficient suppression in the presence of multiple copies of the nucleic acid molecule encoding the suppressor tRNA sequence. In an attempt to reduce the number of viral particles required for efficient suppression, eight copies of the tRNA gene were cloned into pENTR (called pENTR-tRNA8^{TAG}) and recombined into the adenovirus promoterless Destination vector. This new adenovirus (Adeno-tRNA8^{TAG}) was compared with the original monomer virus (Adeno-tRNA^{TAG}) for stop suppression (FIG. 53). As shown by both fluorescent microscopy (upper panels) and anti-β-galactosidase western blotting (lower panel), a modest increase in suppression efficiency was observed with the 8-mer tRNA, and these suppression levels are as good as those seen with the plasmid-based tRNA (lanes 2 and 4). Indeed, in all subsequent experiments, the Ad-tRNA8^{TAG} transduction performed as well or better than a pUC-tRNA^{TAG} plasmid transfection making this recombinant adenovirus configuration particularly suitable for the methods of this invention.

The initial adenovirus experiments used crude adenovirus preparations that still contained all of the debris from the lysed producer cells (roughly 10⁸ cells lysed in 5 ml PBS). This material was functional but resulted in unacceptably high toxicity to the target cells. A variety of purification methods were evaluated to attempt to remove the toxic components from the active adenovirus. Large pore dialysis (300,000 MWCO), sucrose density gradient purification, and HPLC were evaluated for use in the methods of this invention, as were traditional cesium chloride purification and two commercially available adenovirus purification columns (ViraPur, Carlsbad, Calif. and PureSyn, Malvern, Pa.). It was deduced that a modified, single-round cesium chloride step gradient purification (described above) was the least expensive option that gave the highest yields of active virus and exhibited the lowest toxicity on the target cells, making this method particularly suitable for use in the methods of this invention.

Ultimate™ ORF Collection Tag-On-Demand™

The invention described herein is compatible for use with any gene or ORF of interest providing the stop codon is recognized by the provided suppressor tRNA. This stop codon may be native to the gene of interest, or it may be inserted by standard molecular biology techniques, such as described herein. Particularly suited for use in the methods of this invention are clones in the Ultimate™ ORF collection available from Invitrogen Corporation, Carlsbad, Calif. This collection of genes is provided as GATEWAY™ Entry clones containing the native ORF with a TAG stop codon. Tag-On-Demand™ will allow quick and easy detection of expressed protein products (either via V5 or GFP tagging) without needing to generate antibodies against the native protein or recloning the gene to a separate expression vector.

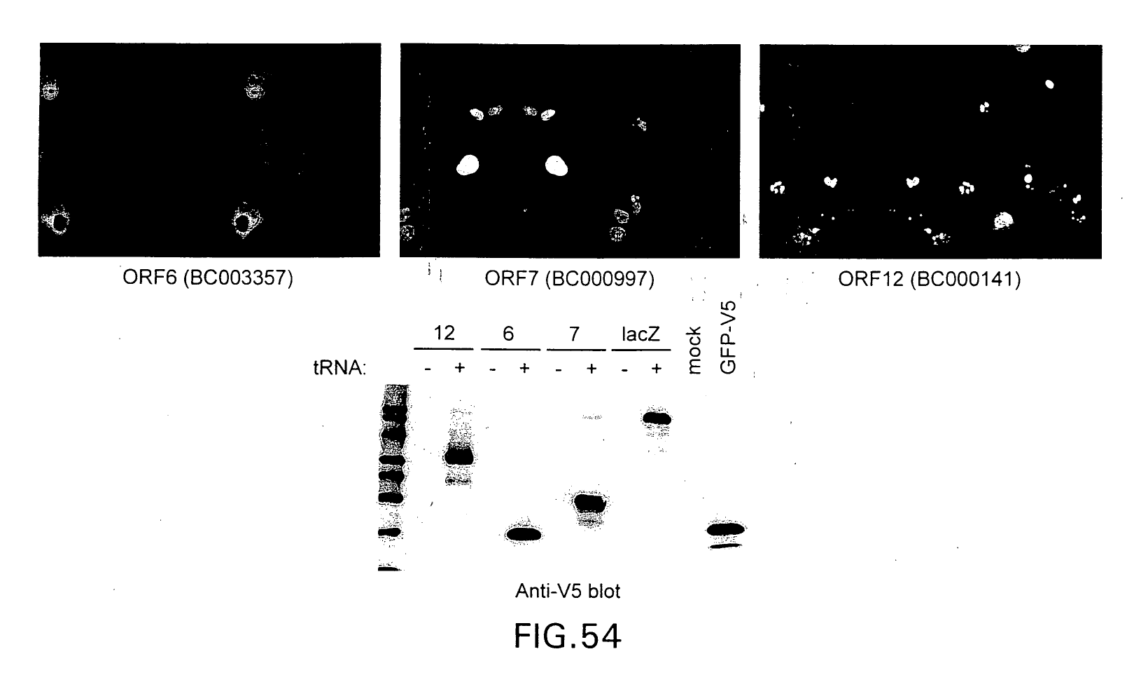

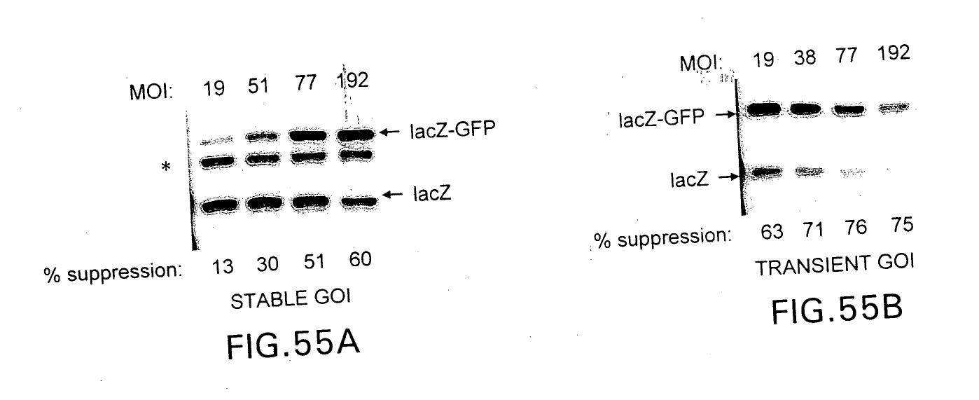

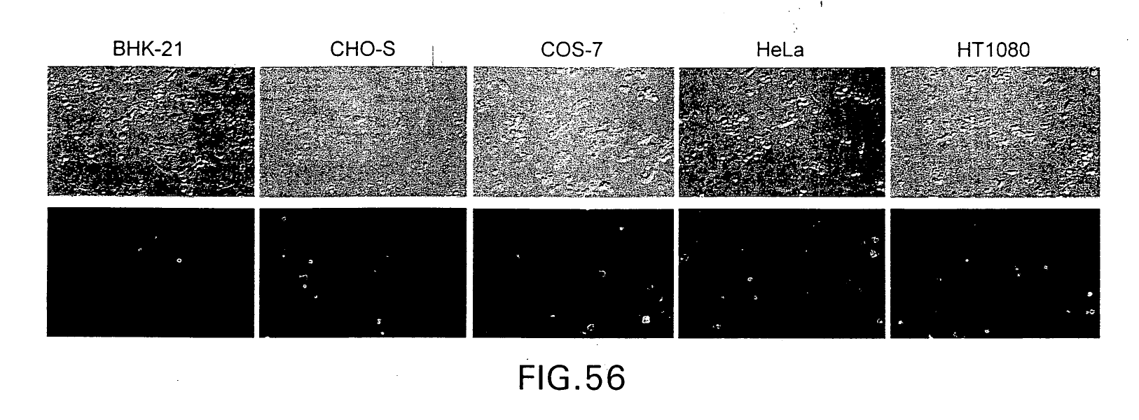

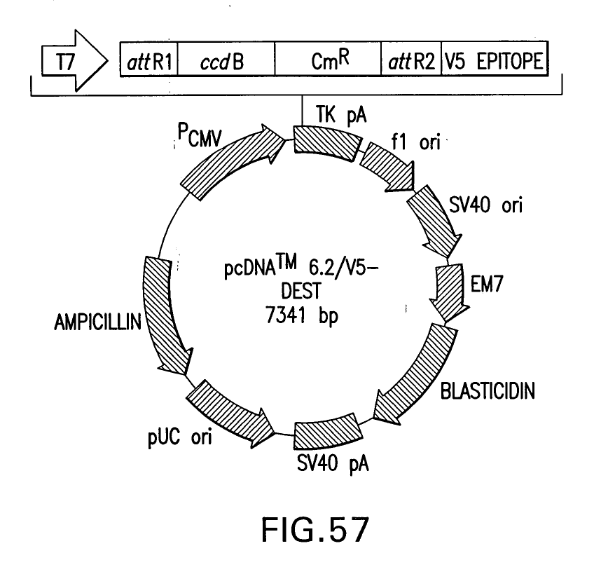

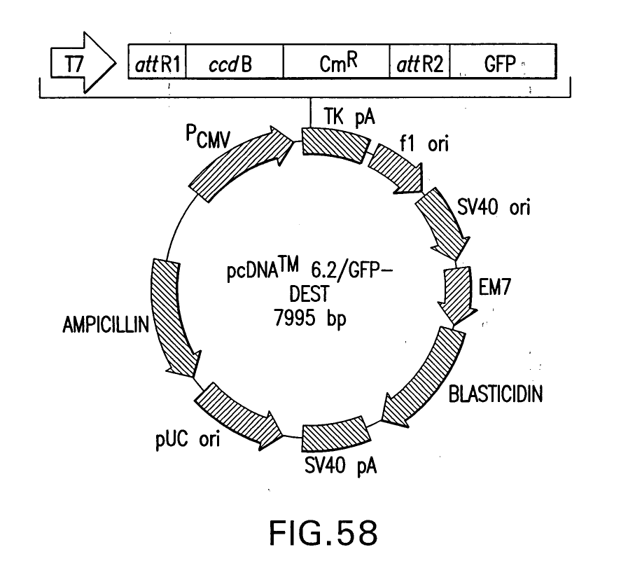

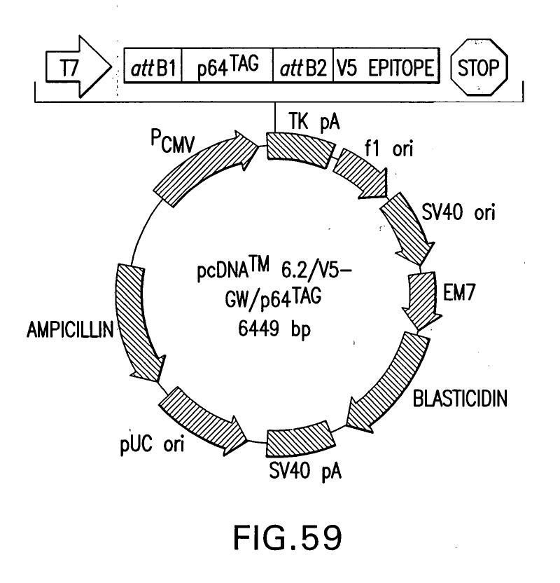

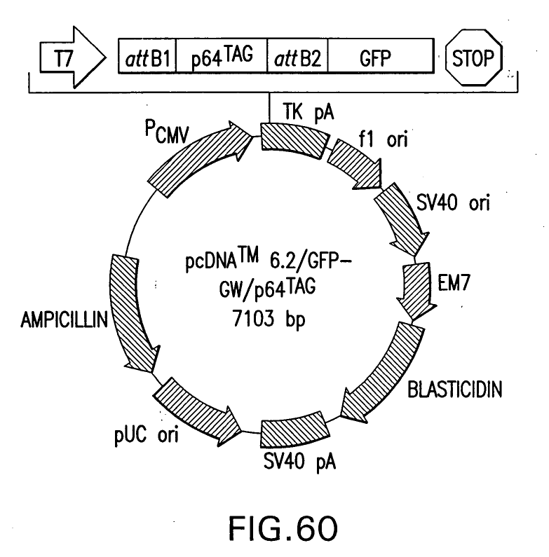

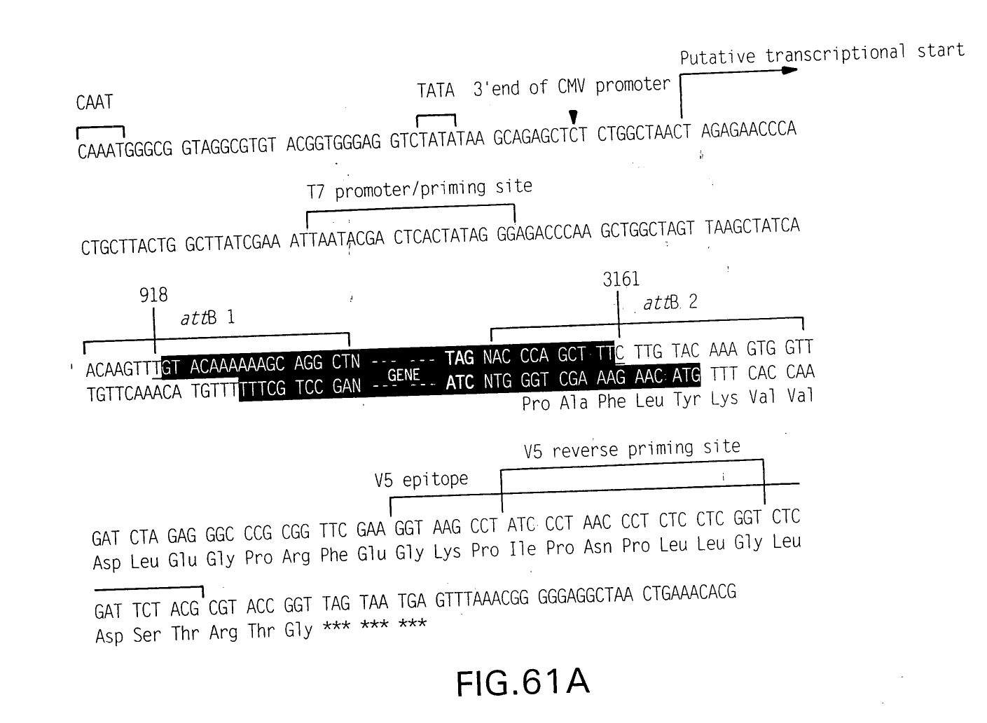

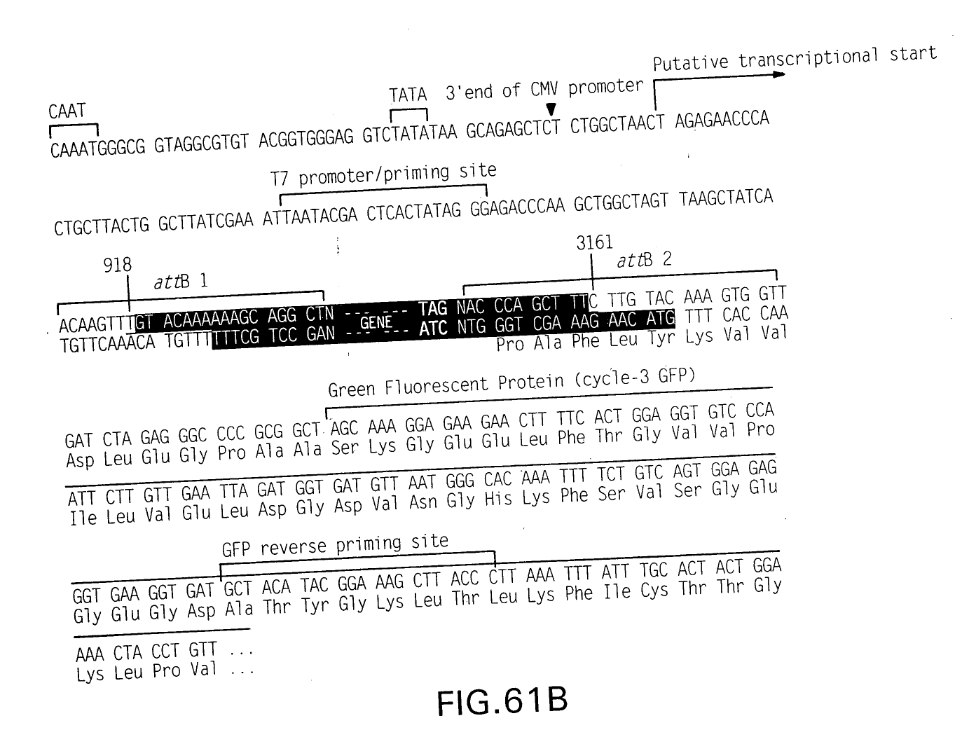

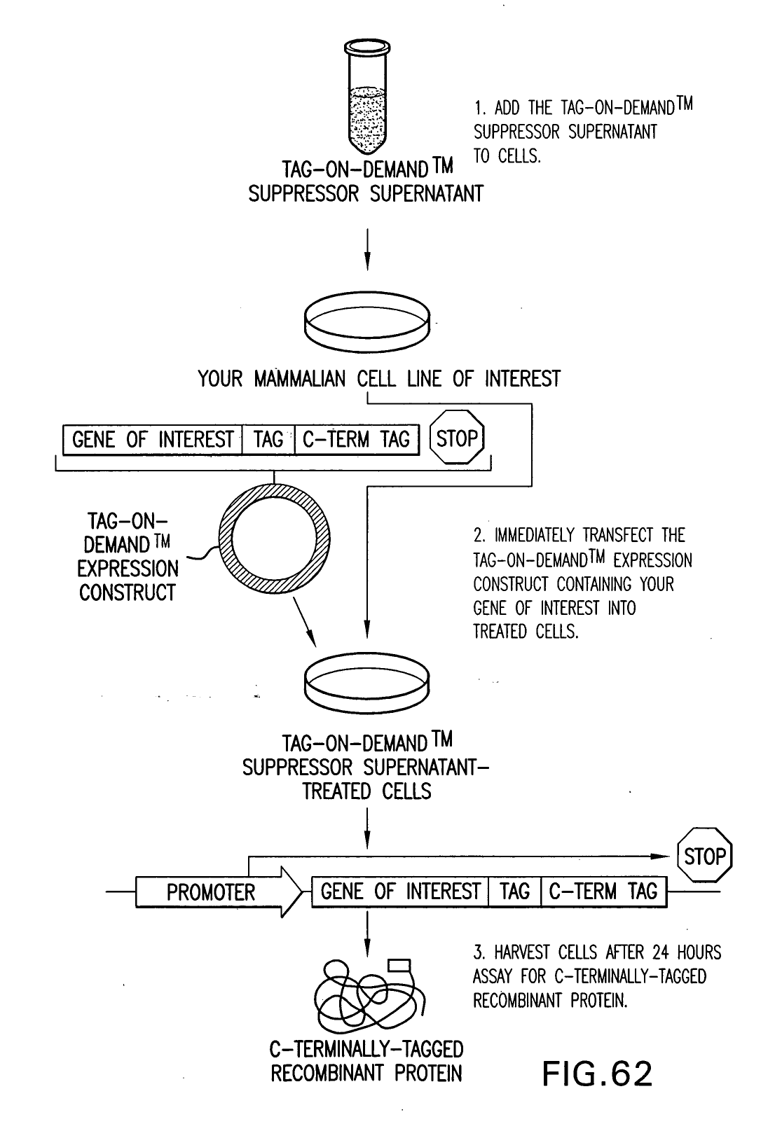

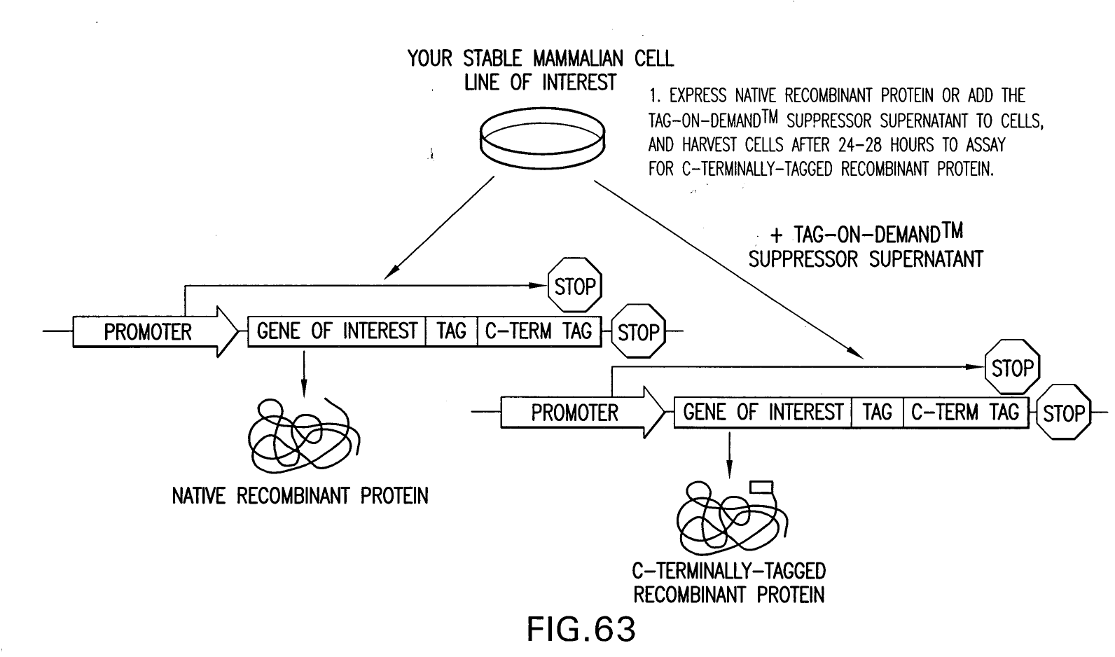

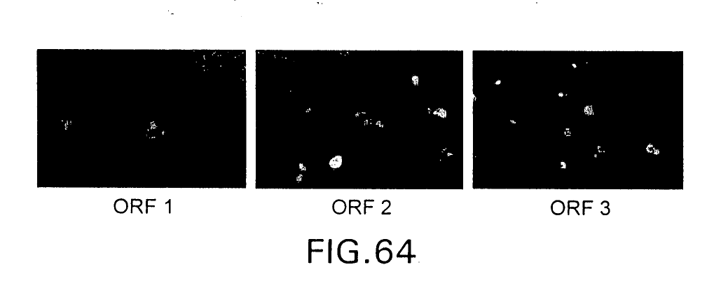

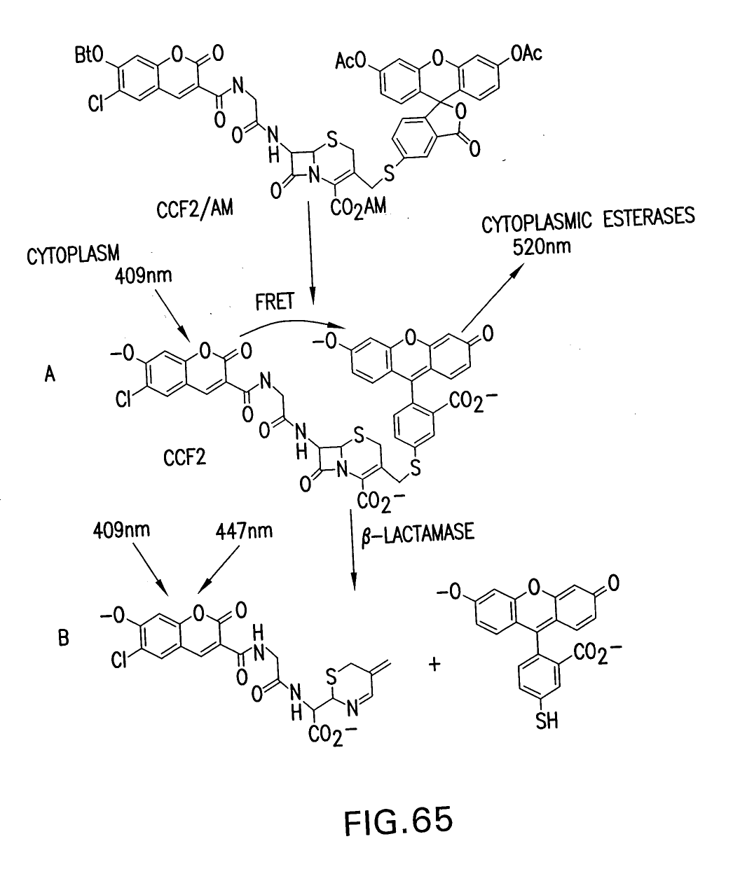

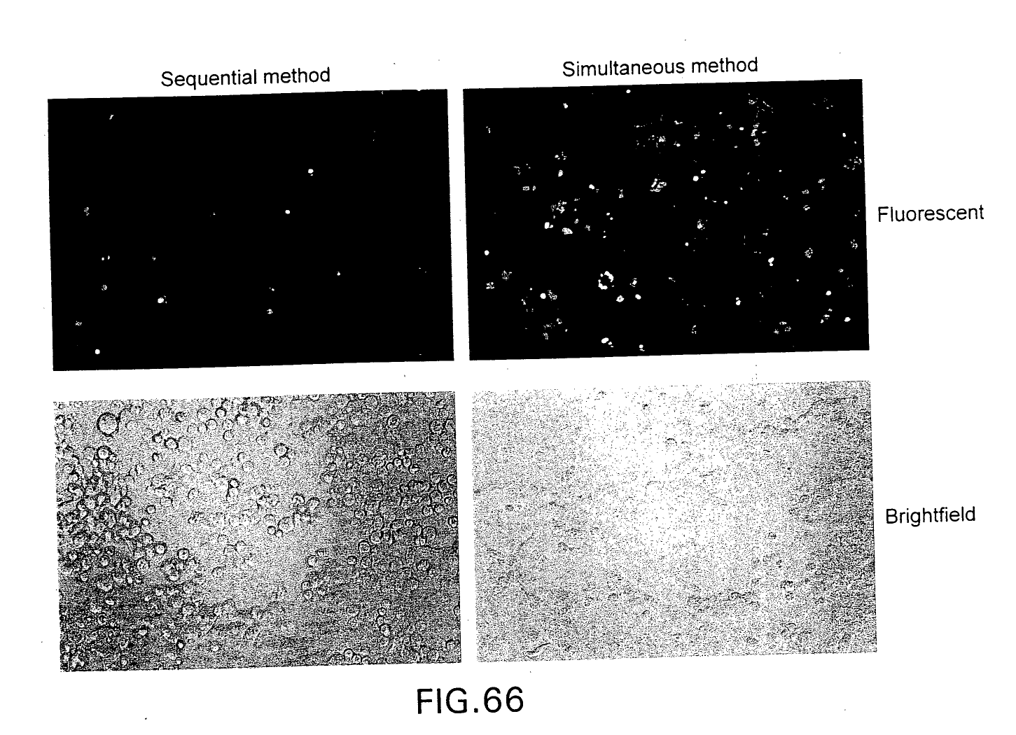

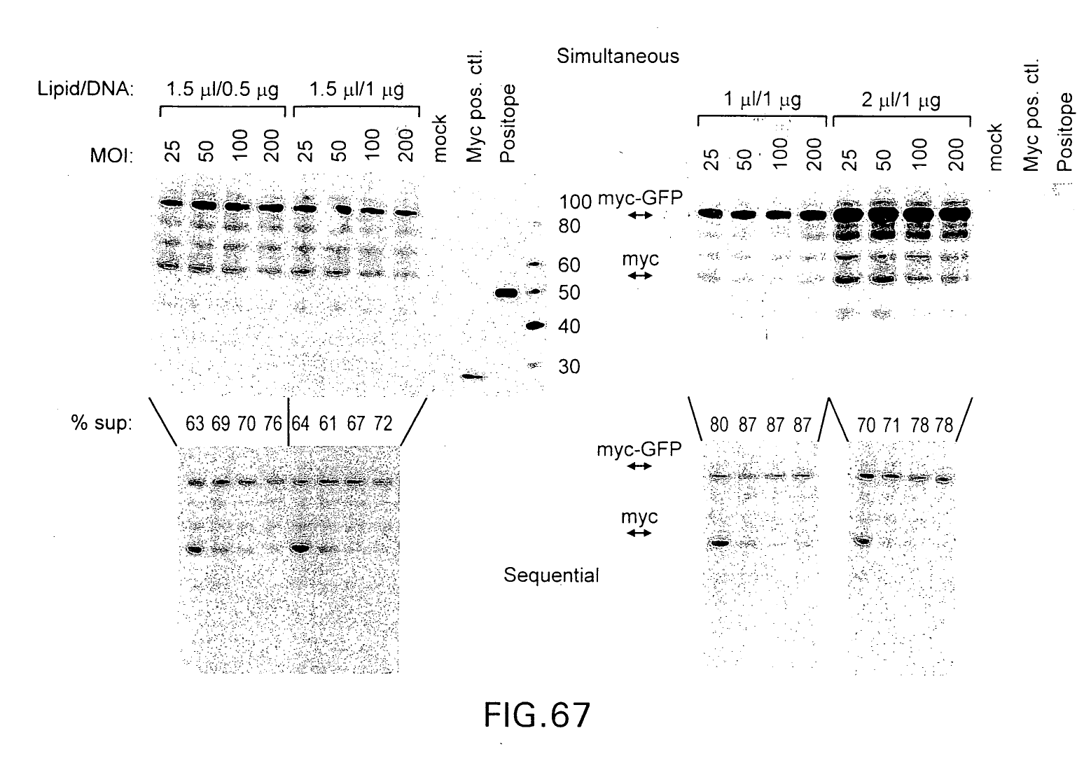

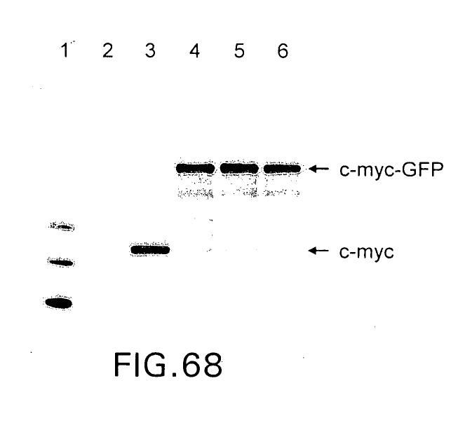

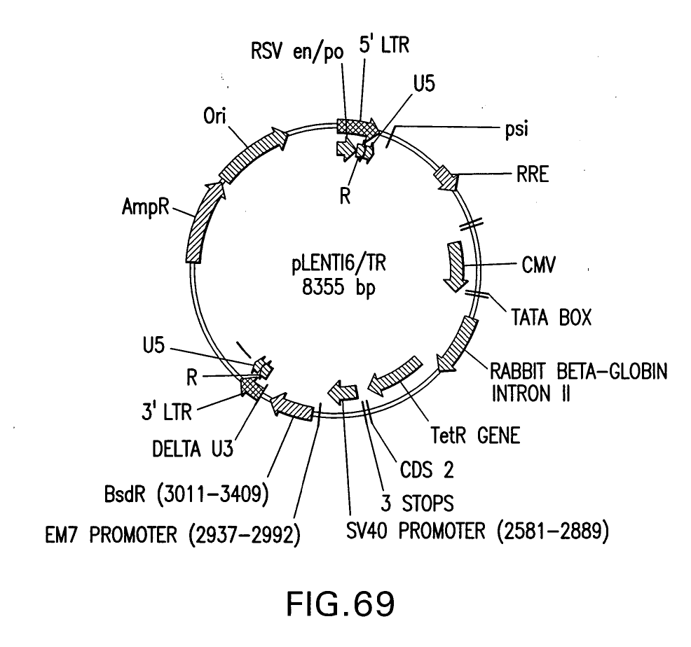

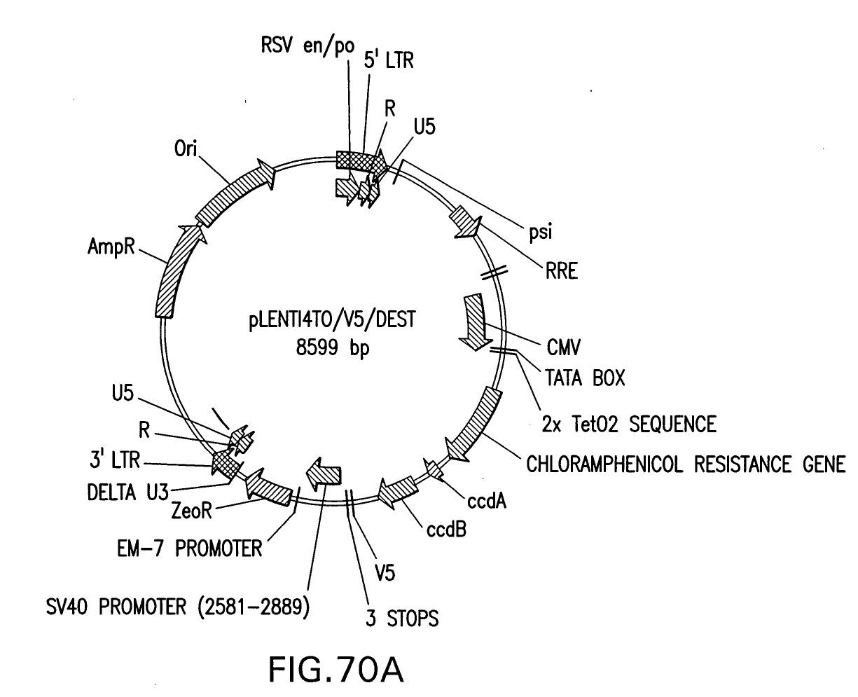

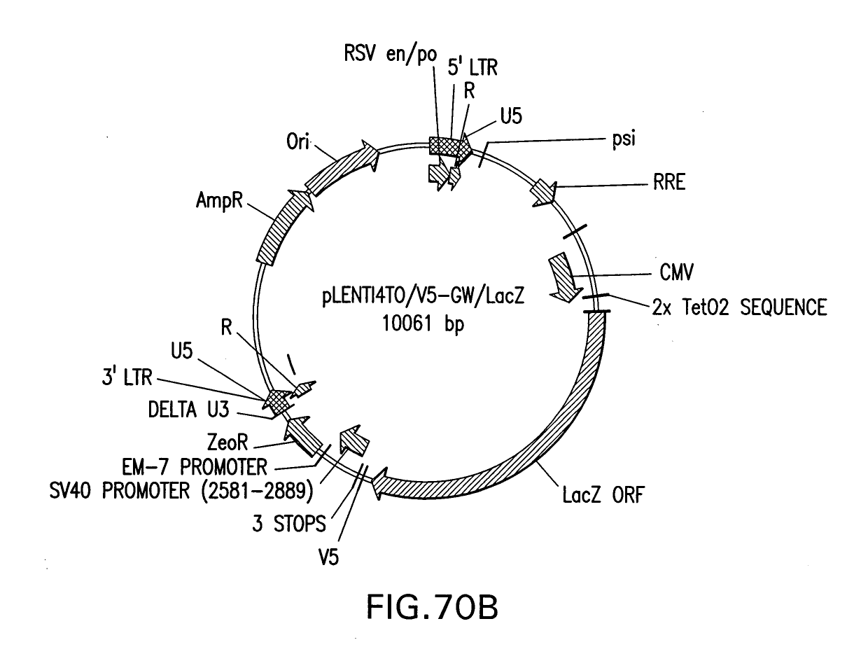

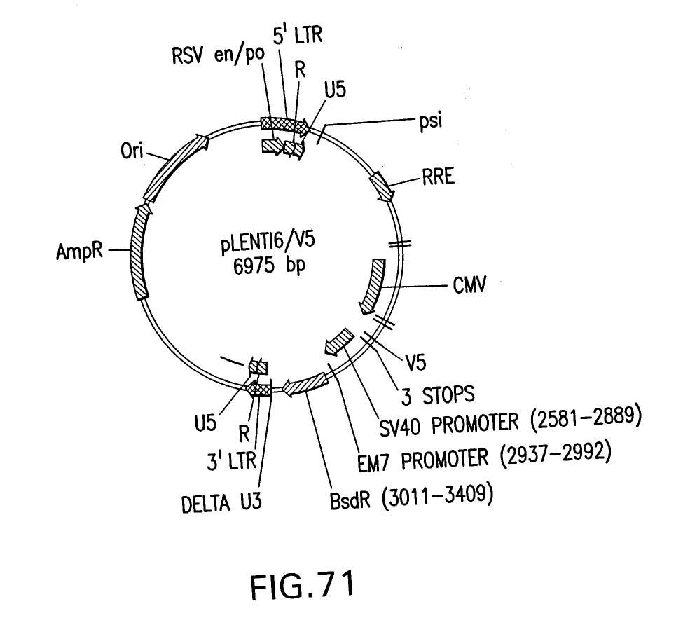

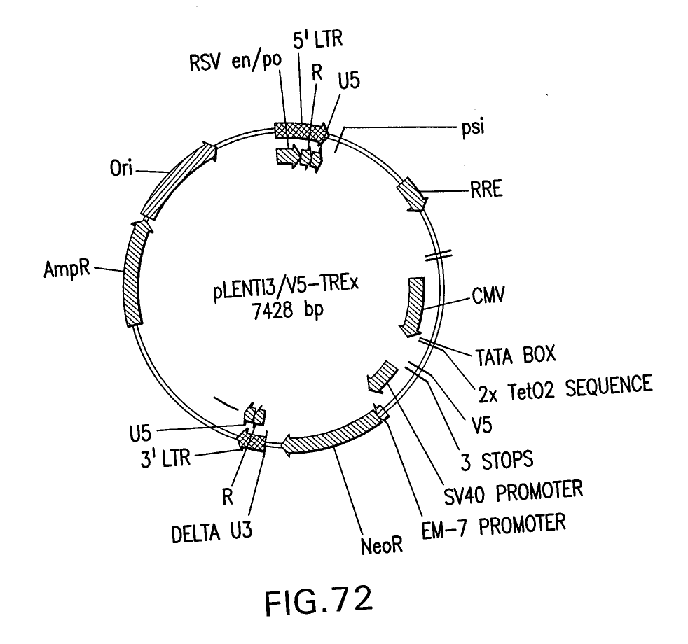

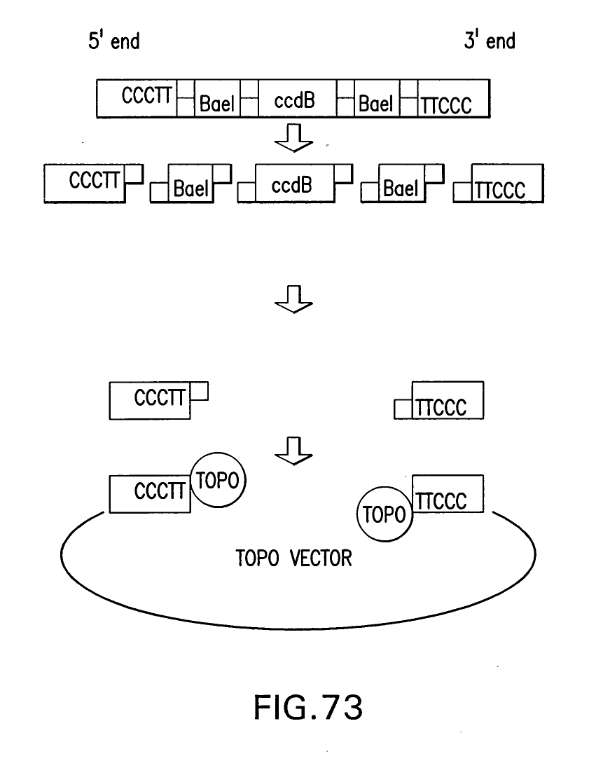

To demonstrate the usefulness of Tag-On-Demand™ with ORFs from the Ultimate™ ORF collection, three human ORFs were chosen and recombined into either pcDNA6.2/GFP-DEST or pcDNA6.2/V5-DEST. Transduction of cells with the Ad-tRNA8^{TAG} followed by transfection of the cells with the ORF-GFP expression clone resulted in easily visible GFP positive cells (FIG. 54, upper panels). Significantly, the proteins retained their normal subcellular localization that was easily detectable using fluorescence microscopy. ORF6 (BC003357) codes for a CGI-130-like protein (23.4 kD) that is primarily cytoplasmic, with nuclear exclusion observed in some cells. ORF7 (BC000997) codes for a human mRNA splicing factor (27.4 kD) and was clearly localized to the nucleus, as expected. ORF12 (BC000141) codes for a truncated form of c-myc (48.8 kD) that is also nuclear, with specific targeting to punctate nucleolar structures. ORF12 is referred to as p48^{TAG} above, and can be the positive expression control for use in kits, as provided in the methods of the present invention. In this example, this experiment was performed by first transducing the cells with adenovirus for 6 hours, followed by transfecting with the reporter plasmid overnight. Alternatively, transduction and transfection may be performed together, or, transfection first followed by transduction. All three methods resulted in good suppression, though transduction followed by transfection to may achieve the best suppression and the least toxicity.

V5 epitope tagging of the ORFs was also successful using the methods of the present invention. Each expressed protein was easily detectable via anti-V5 western blotting, in the presence of the tRNA^{TAG}, and migrated at the correct molecular weight (FIG. 54, lower panel). The addition of the V5 epitope adds approximately 4.2 kD to the protein of interest. ORF-V5 expression levels were comparable to lacZ-stop^{TAG}-V5 suppressed with tRNA^{TAG}, and surprisingly as good as a true V5 fusion protein, GFP-V5, expressed constitutively from pcDNA/GFP-V5 (last lane). This experiment was performed by co-transfection of the ORF-V5 plasmid with pUC12-tRNA^{TAG}, however similar results are obtained using Ad-tRNA8^{TAG}.

It is an important aspect of the invention that ORF expression vectors such as these may express the native protein under normal conditions, allowing the study of its native function. Application of Tag-On-Demand™ allows the use of the exact same expression construct to transiently create tagged versions of the protein. This aspect may be useful for verification of protein expression, analysis of its subcellular localization and even FACSorting of expressing cells without having to generate antibodies to the specific protein or re-cloning the ORF as a true C-terminal fusion.

Tag-On-Demand™ can be used on Both Transient and Stable Gene Targets

One aspect of the present invention is the transient expression of the protein of interest with a tag to verify expression or localization, as described herein. Another aspect of the present invention is to stably express a protein of interest, as demonstrated by the following experiment. Flp-In CHO cells stably expressing a single copy of pcDNA6/FRT/lacZ-stop^{TAG}-GFP were transduced with Adeno-tRNA8^{TAG} at various MOIs (FIG. 55A). Anti-lacZ western blotting revealed a dose-dependent increase in stop suppression with increasing amounts of Adeno-tRNA^{TAG}, and clearly demonstrates that Tag-On-Demand™ can be used to C-terminally tag stably expressed genes. This experiment shows that C-terminally-tagged recombinant protein is produced at all MOIs tested. As the cells are transduced with increasing MOI, the % suppression increases; however, the amount of total recombinant protein produced (untagged and GFP-tagged protein) remains nearly equivalent. The band labeled with an asterisk results from the endogenous lacZ-Zeocin™ fusion present in Flp-In™-CHO cells and is derived from the construct used to create the Flp-In™-CHO cell line.In a parallel experiment, COS cells were transduced with the same range of MOIs for 6 hours, followed by transient transfection of the lacZ-stop^{TAG}-GFP expression vector (FIG. 55B). A dose-dependent increase in suppression with increasing amount of virus was shown, and an MOI as low as 19 can give suppression levels greater than 50%, demonstrating the efficiency of the invention. This experiment shows that at all MOIs tested, the % suppression achieved is >60%, resulting in production of significant levels of GFP-tagged recombinant protein. As the cells are transduced with increasing MOI, the % suppression increases; however, the amount of total recombinant protein produced (untagged and GFP-tagged protein) decreases. This may be indicative of cellular toxicity as a result of the addition of increasing amounts of virus.

Tag-On-Demand™ in Common Mammalian Cell Types

Five commonly used mammalian cells were chosen to evaluate efficiency of Tag-On-Demand™: BHK, CHO, COS, HeLa and HT1080. Cells were transduced with Adeno-tRNA^{TAG} at an MOI of 50 for 6 hours, followed by transient transfection with the lacZ-stop^{TAG}-GFP expression vector. In all cell types tested, Tag-On-Demand™ clearly produced sufficient lacZ-GFP fusion protein to easily detect GFP fluorescence in each cell type (FIG. 56). The slight toxicity observed in these experiments most likely arose from residual cesium chloride in the purified adenovirus preparation.

Summary

One embodiment of the present invention is exemplified by the Tag-On-Demand™ system. Tag-On-Demand™ is a system that uses recombinant adenovirus to deliver a tRNA suppressor gene that results in detection of proteins from the Invitrogen Corporation, Carlsbad, Calif. Ultimate™ ORF collection. As described in the results section, Tag-On-Demand™ is primarily designed for: a) transient detection and localization of expressed protein products, and b) sorting or analysis of expressing cells. Any of the three stop codons normally utilized by cells can be suppressed with the correct tRNA, with TAG stop suppression being most efficient. Adenovirus has been chosen as the tRNA delivery method and was shown to be as good or better than plasmid delivery. Adenovirus is an ideal method for delivering the tRNA genes for a number of reasons:

Adenovirus has broad mammalian cell tropism. Adenovirus binds to the CAR receptor present on most mammalian cells. It is important to note, however, that not all cell types express equal levels of the required CAR receptor, so efficiency may vary from one cell type to another. Fortunately, suppression efficiency can be increased by applying more virus (see FIGS. 55A-B). Like any E1 deleted recombinant adenoviral vectors, use of the Tag-On-Demand™ adenovirus in 293 cells or in any mammalian cell that is expressing the E1 gene of adenovirus will lead to virus replication and possible death of the target cell.

Adenovirus does not stably integrate into the target cell's genome and its expression is transient. Stable delivery or constitutive expression of the tRNA suppressor would most likely be toxic to the cell since one third of the endogenous stop codons would be suppressed, resulting in the addition of extra amino acids to the C-termini of many cellular proteins.

Adenovirus is a very stable virus and can be produced in large quantities. Manufacturing of the virus may include a single round of cesium chloride purification (see Materials and Methods), which yields a pure viral stock with minimal toxicity to the target cell.

In summary, the Tag-On-Demand™ system allows a single expression vector to express either native protein or C-terminally tagged protein. The system is completely compatible with the GATEWAY™ cloning technology and Ultimate™ ORF collection. The present invention is particularly suited for use with the pcDNA6.2/V5 and pcDNA6.2/GFP Destination vectors, however a person of skill in the art would readily recognize that a variety of other Invitrogen vectors as well as others are also compatible with Tag-On-Demand™, including many TOPO vectors, myc and 6×-His vectors, T-REx and Flp-In as described in the Materials and Methods. Further provided in the present invention is the ability to clone any downstream “tag” for fusing to any protein of interest, provided there is a non-TAG stop codon at the end of the C-terminal tag.

### Example 15

### Tag-on-Demand™ GATEWAY® Vectors

In some embodiments, the present invention provides nucleic acid molecules (e.g., vectors) that may be used to express fusion polypeptides (e.g., polypeptides comprising a sequence of interest and at least one additional polypeptide sequence). Non-limiting examples of vectors suitable for use in the present invention are GATEWAY®-adapted destination vectors. Such vectors may be used for high-level expression of native and C-terminally-tagged polypeptides from the same nucleic acid molecules. In some embodiments, such vectors may be used to express polypeptides (which may be fusion polypeptides) in mammalian cells. Such vectors may be used to express fusion polypeptides by introducing the vectors into a host cell and also introducing into the host cell a source of a suppressor tRNA. Any suitable source of a suppressor tRNA may be used (e.g., plasmids, linear nucleic acid molecules, viruses, etc.) In some embodiments, the present invention provides an adenovirus that expresses one or more suppressor tRNA molecules and/or one or more copies of a suppressor tRNA molecule. Methods that employ an adenovirus expressing tRNAs may be referred to herein as the Tag-on-Demand™ System.

One or more of the following commercially available items may be used in connection with the methods of the invention. These items are listed with their Invitrogen Corporation, Carlsbad, Calif. catalog number in parenthesis: Tag-On-Demand™ Suppressor Supernatant (K400-01 or K405-01); Gateway® LR Clonase™ Enzyme Mix (11791-019 or 11791-043); Library Efficiency® DB3.1™ Competent Cells (11782-018); One Shot® TOP10 Chemically Competent E. coli (C4040-03); Library Efficiency DH5α™ Chemically Competent E. coli (18263-012); Blasticidin (R210-01); Ampicillin (Q100-16); S.N.A.P.™ MidiPrep Kit (K1910-01); Lipofectamine™ 2000 (11668-027 or 11668-019); and Phosphate-Buffered Saline (PBS), pH 7.4 (10010-023).

In some embodiments, the present invention provides methods of producing fusion polypeptides comprising a polypeptide sequence of interest fused to one or more additional polypeptide sequences. If a fusion polypeptide is produced (e.g., from pcDNA™6.2/V5-DEST or pcDNA™6.2/GFP-DEST), the fusion polypeptide can be detected using an antibody that binds to one or more of the additional polypeptide sequences (e.g., to the V5 epitope or to GFP). Commercially available antibody preparations can be used, for example, those available from Invitrogen Corporation, Carlsbad, Calif. such as Anti-V5 Antibody (catalog # R960-25), Anti-V5-HRP Antibody (catalog # R961-25), Anti-V5-AP Antibody (catalog # R962-25), Anti-V5-FITC Antibody (catalog # R963-25), GFP Antiserum (catalog # R970-01).

In some embodiments, methods of the invention may be used to express a fusion polypeptide comprising all or a portion of p64. To detect the p64 (human c-myc) protein expressed using methods and materials of the invention, commercially available Anti-myc Antibodies may be used (e.g., Invitrogen Corporation, Carlsbad, Calif. Anti-myc Antibody catalog no. R950-25, Anti-myc-HRP Antibody catalog no. R951-25, Anti-myc-AP Antibody catalog no. R952-25, and/or Anti-myc-FITC Antibody catalog no. R953-25).

Examples of nucleic acid molecules that may be used in the practice of the invention include, but are not limited to, pcDNA™6.2/V5-DEST (7.3 kb) and pcDNA™6.2/GFP-DEST (8.0 kb), which are destination vectors adapted for use with GATEWAY® Technology (Invitrogen Corporation, Carlsbad, Calif.) and allow high-level, constitutive expression of recombinant polypeptides in mammalian cells. The vectors are designed for use with a suppressor tRNA producing nucleic acid molecule (e.g., Invitrogen's Tag-on-Demand™ System), which allows expression of both native and C-terminally-tagged recombinant polypeptide from the same expression construct.

The pcDNA™6.2/V5-DEST and pcDNATm6.2/GFP-DEST vectors enable expression of recombinant polypeptide containing a choice of C-terminal tags. The pcDNATm6.2/V5-DEST vector encodes the V5 epitope for detection of recombinant polypeptide using the Anti-V5 antibodies. A plasmid map is provided as FIG. 57 and the sequence of this vector is provided as Table 28. The pcDNA™6.2/GFP-DEST vector encodes the Cycle-3 GFP for fusion to a polypeptide sequence of interest and use as a reporter gene. A plasmid map of this vector is provided as FIG. 58 and the sequence of this vector is provided as Table 29.

The pcDNA™6.2/V5-DEST and pcDNA™6.2/GFP-DEST vectors contain the following features: human cytomegalovirus (CMV) immediate early promoter for high-level constitutive expression of the gene of interest in a wide range of mammalian cells (Andersson, S., et aL, J. Biol. Chem. 264:8222-8229 (1989); Boshart, M., et al., Cell 41:521-530 (1985); Nelson, J. A., et al., Molec. Cell. Biol. 7:4125-4129 (1987)); two recombination sites, attR1 and attR2, downstream of the CMV promoter for recombinational cloning of the DNA sequence of interest from an entry clone; the chloramphenicol resistance gene (Cm^{R}) located between the two attR sites for counterselection; the ccdB gene located between the attR sites for negative selection; the C-terminal V5 epitope for detection of the recombinant polypeptide of interest (in pcDNA™6.2/V5-DEST only) (Southern, J. A., et al., J. Gen. Virol. 72:1551-1557 (1991)); the C-terminal cycle-3 Green Fluorescent Protein (GFP) gene for fusion of the recombinant polypeptide of interest to a reporter (in pcDNA™6.2/GFP-DEST only) (Chalfie, M., et al., Science 263:802-805 (1994); Crameri, A., et al., Nature Biotechnol. 14:315-319 (1996)); the Herpes Simplex Virus thymidine kinase (TK) polyadenylation sequence for efficient transcription termination and polyadenylation of mRNA (Cole, C. N., and Stacy, T. P., Mol. Cell. Biol. 5:2104-2113 (1985)); the Blasticidin resistance gene for selection of stable cell lines (Kimura, M., et al., Biochim. Biophys. ACTA 1219:653-659 (1994)); the pUC origin for high-copy replication and maintenance of the plasmid in E. coli; the ampicillin (bla) resistance gene for selection in E. coli. In one alternative of this aspect of the invention, the chloramphenicol resistance gene in the cassette can be replaced by a spectinomycin resistance gene (see Hollingshead et al., Plasmid 13(1):17-30 (1985), NCBI accession no. X02340 M10241), and the pcDNA destination vector containing attP sites flanking the ccdB and spectinomycin resistance genes can be selected on ampicillin/spectinomycin-containing media. Use of spectinomycin selection instead of chloramphenicol selection may result in an increase in the number of colonies obtained on selection plates, indicating that use of the spectinomycin resistance gene may lead to an increased efficiency of cloning from that observed using cassettes containing the chloramphenicol resistance gene.

The location in the plasmid sequence of pcDNA™6.2/V5-DEST (7341 nucleotides) of the features discussed above are: CMV promoter bases 232-819; T7 promoter/priming site bases 863-882; attR1 site bases 911-1035; ccdb gene bases 1464-1769 (c); chloramphenicol resistance gene bases 2111-2770 (c); attR2 site bases 3051-3175; V5 epitope bases 3201-3242; V5 reverse priming site 3210-3230; TK polyadenylation signal bases 3269-3540; fl origin 3576-4004; SV40 early promoter and origin 4031-4339; EM7 promoter bases 4394-4460; Blasticidin resistance gene bases 4461-4859; SV40 early polyadenylation signal bases 5017-5147; pUC origin bases 5530-6200 (c); Ampicillin (bla) resistance gene bases 6345-7205 (c); bla promoter bases 7206-7304 (c) where (c) indicates present on the complementary strand.

The location in the plasmid sequence of pcDNA™6.2/GFP-DEST (7995 nucleotides) of the features discussed above are: CMV promoter bases 232-819; T7 promoter/priming site bases 863-882; attR1 site bases 911-1035; ccdB gene bases 1464-1769 (c); Chloramphenicol resistance gene bases 2111-2770 (c); attR2 site bases 3051-3175; Cycle-3 GFP bases 3195-3908; GFP reverse priming site 3303-3324; TK polyadenylation signal bases 3923-4194; fl origin 4230-4658; SV40 early promoter and origin 4685-4993; EM7 promoter bases 5048-5114; Blasticidin resistance gene bases 5115-5513; SV40 early polyadenylation signal bases 5671-5801; pUC origin bases 6184-6854 (c); Ampicillin (bla) resistance gene bases 6999-7859 (c); bla promoter bases 7860-7958 (c), where (c) indicates the feature is present on the complementary strand.

In some embodiments, positive control nucleic acid molecules (e.g., plasmids may be used in conjunction with the methods of the invention. A suitable positive control nucleic acid molecule is one comprising a nucleic acid sequence encoding two polypeptide sequences in the same reading frame and having a stop codon in between the sequences. For example, the polypeptide encoded 3′ to the stop codon may have a detectable activity (i.e., enzymatic activity, fluorescent activity, binding activity, etc.). Examples of suitable control nucleic acid molecules include, but are not limited to, pAd/CMV/V5-GW/lacZ, pcDNA™6.2/V5-GW/p64^{TAG} and pcDNA™6.2/GFP-GW-p64^{TAG}, which were prepared from the corresponding vectors by conducting an L×R reaction with an entry vector containing the indicated coding sequence (i.e., lacZ or p64 coding sequence (also known as c-myc). Plasmid maps of the control vectors pcDNA™6.2/V5-GW/p64^{TAG} and pcDNA™6.2/GFP-GW-p64^{TAG} are provided as FIGS. 59 and 60 respectively.

The GFP gene used in the pcDNA™6.2/GFP-DEST vector is described in Crameri, A., et al., Nature Biotechnol. 14:315-319 (1996). In this paper, the codon usage was optimized for expression in E. coli and three cycles of DNA shuffling were used to generate a mutant form of GFP that expresses well in mammalian cells and has excitation and emission maxima that are the same as wild-type GFP (395 nm and 478 nm for primary and secondary excitation, respectively, and 507 nm for emission) and a >40-fold increase in fluorescent yield over wild-type GFP. This mutant GFP is referred to as Cycle-3 GFP to differentiate it from wild-type GFP.

Materials and methods of the invention (e.g., The Tag-on-Demand™ System, Invitrogen Corporation, Carlsbad, Calif.) facilitate transient expression of C-terminally-tagged and untagged recombinant polypeptides from a single expression construct such as one prepared using GATEWAY™. The System is based on stop suppression technology originally developed by RajBhandary and colleagues (Capone, J. P., et al., EMBO J. 4:213-221 (1985)), and consists of two major components: an expression vector into which the gene of interest will be cloned and a nucleic acid molecule (or composition comprising such a nucleic acid molecule) expressing one or more suppressor tRNAs (e.g., the Tag-on-Demand™ Suppressor Supernatant). The vector (e.g., pcDNA™6.2/V5-DEST or pcDNA™6.2/GFP-DEST) must be in a configuration that is compatible with expression of C-terminally-tagged recombinant polypeptide by introducing a suppressor tRNA to suppress a stop codon (e.g., by using the Tag-on-Demand™ System). In one non-limiting embodiment, (i.e., the Tag-on-Demand™ Suppressor Supernatant) a suppressor tRNA molecule may be introduced into a host cell by transducing the host cell with a replication-incompetent adenovirus containing the human tRNA^{ser} suppressor. This tRNA suppressor has been mutated to recognize the TAG (amber stop) codon and decode it as a serine. When added to mammalian cells, the Tag-on-Demand™ Suppressor Supernatant is transduced and provides a transient source of the tRNA^{ser} suppressor.

When an expression vector encoding a gene of interest with the TAG stop codon is transfected into mammalian cells, the stop codon will be translated as serine, allowing translation to continue through any downstream reading frame (e.g., a C-terminal tag), and resulting in production of a fusion polypeptide containing the polypeptide encoded by the gene of interest fused to the amino acids encoded 3′ to the stop codon (e.g., a marker or tag sequence). One skilled in the art will appreciate that, in similar fashion, a nucleic acid molecule (e.g., a replication-incompetent adenovirus) expressing a suppressor tRNA that suppresses TAA (ochre) or TGA (opal) stop codons can be prepared and used in the practice of the present invention.

To recombine a DNA sequence of interest into a nucleic acid molecule of the invention (e.g., pcDNATm6.2/V5-DEST or pcDNA™6.2/GFP-DEST), an entry clone containing the DNA comprising a sequence of interest may prepared. In an entry clone, a sequence of interest may be flanked by recombination sites (e.g., sites compatible with those in one or more destination vector). Many entry vectors are available from Invitrogen to facilitate generation of entry clones. Examples include, but are not limited to, pENTRID-TOPO® (catalog number K2400-20), pENTR/SD/D-TOPO® (catalog number K2420-20), pENTR™1A (catalog number 11813-011), pENTR™2B (catalog number 11816-014), pENTR™3C (catalog number 11817-012), pENTR™4 (catalog number 11818-010), and pENTR™11 (catalog number 11819-018).

In some embodiments, the present invention encompasses the expression of fusion polypeptides comprising all or a portion of a human polypeptide. One suitable source of nucleic acid molecules encoding human polypeptides is the Ultimate™ Human ORF (hORF) Clone collection available from Invitrogen Corporation, Carlsbad, Calif. To express a human gene of interest from pcDNA™6.2/V5-DEST or pcDNA™6.2/GFP-DEST, an Ultimate™ Human ORF (hORF) Clone available from Invitrogen Corporation, Carlsbad, Calif. can be used. Each Ultimate™ hORF Clone is a fully-sequenced clone provided in a GATEWAY® entry vector that is ready-to-use in an LR recombination reaction with pcDNA™6.2/V5-DEST or pcDNA™6.2/GFP-DEST. In addition, each Ultimate™ hORF Clone contains a TAG stop codon, making it fully compatible for use in the Tag-on-Demand™ System. For more information about the Ultimate™ hORF Clones available, see the Invitrogen Corporation, Carlsbad, Calif. Web site or contact Invitrogen Corporation, Carlsbad, Calif.

When generating an entry clone, a nucleic acid sequence encoding a polypeptide of interest in the entry clone must contain an ATG initiation codon in the context of a Kozak consensus sequence for proper initiation of translation in mammalian cells as discussed above.

To enable expression of both a native and C-terminally-tagged recombinant polypeptide of interest from pcDNA™6.2/V5-DEST or pcDNA™6.2/GFP-DEST using the Tag-on-Demand™ System, the gene of interest in the entry clone may contain a stop codon. This stop codon may be encoded by the nucleotides, TAG. In addition, the gene should be in frame with the C-terminal tag after recombination. Those skilled in the art will appreciate that other stop codons can be similarly used by constructing a vector expressing a suppressor tRNA that recognizes the other stop codons.

The recombination region of pcDNA™6.2/V5-DEST and pcDNA6.2/GFP-DEST are provided as FIGS. 61A and 61B respectively. In FIG. 61A, shaded regions correspond to those DNA sequences transferred from the entry clone into the pcDNA™6.2/V5-DEST vector by recombination. Non-shaded regions are derived from the pcDNA™6.2/V5-DEST vector. The sequences encoded by the gene of interest are boxed. To facilitate use with the Tag-on-Demand™ System, a gene of interest must contain a TAG stop codon and be in-frame with the C-terminal tag. Bases 918 and 3161 of the pcDNA™6.2/V5-DEST sequence are marked. Note that TAA and TGA stop codons are included downstream of the V5 epitope to allow translation termination in the Tag-on-Demand™ System. In FIG. 61B, the recombination region of the expression clone resulting from pcDNA™6.2/GFP-DEST× entry clone is shown. The shaded regions correspond to those DNA sequences transferred from the entry clone into the pcDNA™6.2/GFP-DEST vector by recombination. Non-shaded regions are derived from the pcDNA™6.2/GFP-DEST vector. The sequences encoded by the gene of interest are boxed. To facilitate use with the Tag-on-Demand™ System, the gene of interest should contain a TAG stop codon. Bases 918 and 3161 of the pcDNA™6.2/GFP-DEST sequence are marked. TAA and TGA stop codons are included downstream of the GFP gene to allow translation termination in the Tag-on-Demand™ System (not shown).

To generate an expression clone: an LR recombination reaction using the attL-containing entry clone and the attR-containing pcDNA™6.2/V5-DEST or pcDNA™6.2/GFP-DEST vector may be performed. Both the entry clone and the destination vector may be supercoiled or linear. After the LR reaction has been performed, all or a portion of the reaction mixture may be used to transform a suitable E. coli host. The expression clones can be selected for using ampicillin and/or blasticidin.

The pcDNA™6.2/V5-DEST and pcDNA™6.2/GFP-DEST vectors are supplied as supercoiled plasmids. Although the GATEWAY® Technology manual has previously recommended using a linearized destination vector for more efficient recombination, it has been found that linearization of pcDNA™6.2/V5-DEST and pcDNA™6.2/GFP-DEST is not required to obtain optimal results for any downstream application.

Nucleic acid molecules of the invention, (e.g., destination vectors) may be lyophilized for long term storage. Lyophilized plasmids may be resuspended in a suitable buffer (e.g., TE, pH 8.0). In some embodiments, the vectors may be lyophilized in a buffer (e.g., TE, pH 8.0) and may be resuspended by the addition of sterile water. A suitable concentration for solutions of nucleic acid molecules to be used in the practice of the invention is about 150 ng/μl although other concentrations may be used.

In some embodiments, nucleic acid molecules of the invention may be propagated in suitable host cells. To propagate and maintain the pcDNA™6.2/V5-DEST or pcDNA™6.2/GFP-DEST vectors, Library Efficiency® DB3.1™ Competent Cells (Invitrogen Corporation, Carlsbad, Calif. Catalog no. 11782-018) can be used. The DB3.1™ E. coli strain is resistant to CcdB effects and can support the propagation of plasmids containing the ccdB gene. To maintain integrity of the vector, select for transformants in media containing 50-100 μg/ml ampicillin and 15-30 μg/ml chloramphenicol. General E. coli cloning strains including TOP10 or DH5α should not be used for propagation and maintenance as these strains are sensitive to CcdB effects.

Once an entry clone containing a gene of interest has been prepared, perform an LR recombination reaction between the entry clone and pcDNA™6.2/V5-DEST or pcDNA™6.2/GFP-DEST, and transform the reaction mixture into a suitable E. coli host. A negative control (no entry vector) is recommended to help evaluate results. Any recA, endA E. coli strain including TOP10, DH5α™, or equivalent for transformation can be used. Do not transform the LR reaction mixture into E. coli strains that contain the F′ episome (e.g., TOP10 F′). These strains contain the ccdA gene and will prevent negative selection with the ccdb gene.

The pcDNA™6.2/V5-DEST and pcDNA™6.2/GFP-DEST vectors contain the ampicillin and Blasticidin resistance genes to allow selection of E. coli transformants using ampicillin or Blasticidin, respectively. To select for transformants using Blasticidin, use Low Salt LB agar plates containing 100 μg/ml Blasticidin. For Blasticidin to be active, the salt concentration of the medium must remain low (<90 mM) and the pH must be 7.0. Low salt plates may be prepared by mixing 10 g Tryptone, 5 g NaCl, 5 g Yeast Extract and adding deionized, distilled water to 950 ml. Adjust pH to 7.0 with 1 N NaOH. Bring the volume up to 1 liter. For plates, add 15 g/L agar. before autoclaving.

Autoclave on liquid cycle at 15 psi and 121° C. for 20 minutes. Allow the medium to cool to at least 55° C. before adding the blasticidin to 100 μg/ml final concentration. Store plates at +4° C. in the dark. Plates containing blasticidin are stable for up to 2 weeks. Blasticidin is available from Invitrogen Corporation, Carlsbad, Calif.

An LR recombination reaction may be performed with purified plasmid DNA of an entry clone (50-150 ng/μl in TE, pH 8.0); pcDNA™6.2/V5-DEST or pcDNA™6.2/GFP-DEST vector (150 ng/μl in TE, pH 8.0); LR Clonase™ enzyme mix (Invitrogen, Catalog no. 11791-019; keep at −80° C. until immediately before use); 5× LR Clonase™ Reaction Buffer (supplied with the LR Clonase™ enzyme mix); TE Buffer, pH 8.0 (10 mM Tris-HCl, pH 8.0, 1 mM EDTA); 2 μg/μl Proteinase K solution (supplied with the LR Clonase™ enzyme mix; thaw and keep on ice until use); an appropriate competent E. coli host and growth media for expression; SOC Medium; and selective plates (e.g., LB agar plates containing 100 μg/ml ampicillin or Low Salt LB plates containing 100 82 g/ml Blasticidin).

Add the following components to 1.5 ml microcentrifuge tubes at room temperature and mix.

| Component | Sample | | Negative Control | |
|---|---|---|---|---|
| Entry clone (100-300 ng/reaction) | 1-10 | μl | — | |
| Destination vector (300 ng/reaction) | 2 | μl | 2 | μl |
| 5 × LR Clonase ™ Reaction Buffer | 4 | μl | 4 | μl |
| TE Buffer, pH 8.0 | to 16 | μl | 10 | μl |

Remove the LR Clonase™ enzyme mix from −80° C. and thaw on ice (˜2 minutes). Vortex the LR Clonase™ enzyme mix briefly twice (2 seconds each time). To each sample above, add 4 μl of LR Clonase™ enzyme mix. Mix well by pipetting up and down. Return LR Clonase™ enzyme mix to −80° C. immediately after use. Incubate reactions at 25° C. for 1 hour. Extending the incubation time to 18 hours typically yields more colonies. Add 2 μl of the Proteinase K solution to each reaction. Incubate for 10 minutes at 37° C. Transform 1 μl of the LR recombination reaction into a suitable E. coli host (follow the manufacturer's instructions) and select for expression clones. The LR reaction may be stored at −20° C. for up to 1 week before transformation, if desired.

If E. coli cells with a transformation efficiency of 1×10⁸ cfu/mg are used, the LR reaction should give approximately >5,000 colonies if the entire transformation is plated.

The ccdB gene mutates at a very low frequency, resulting in a very low number of false positives. True expression clones will be ampicillin-resistant and chloramphenicol-sensitive. Transformants containing a plasmid with a mutated ccdB gene will be ampicillin- and chloramphenicol-resistant. To check a putative expression clone, test for growth on LB plates containing 30 μg/ml chloramphenicol. A true expression clone should not grow in the presence of chloramphenicol.

To confirm that a gene of interest is in the correct orientation and in frame with the C-terminal fusion tag, the expression construct can be sequenced. The following primers can be used to sequence an expression construct. FIGS. 61A and 61B provide the location of the primer binding sites in each vector. For sequencing the pcDNA™6.2/V5-DEST vector, an oligonucleotide that binds to the T7 promoter/priming site (e.g., 5′-TAATACGACTCACTATAGGG-3′) and an oligonucleotide that binds to the V5(C-term) reverse priming site (e.g., 5′-ACCGAGGAGAGGGTTAGGGAT-3′) can be used. To sequence the pcDNA™6.2/GFP-DEST vector, an oligonucleotide that binds to the T7 promoter/priming site (e.g., 5′-TAATACGACTCACTATAGGG-3′) and an oligonucleotide that binds to the GFP reverse priming site (e.g., 5′-GGGTAAGCTTTCCGTATGTAGC-3′) can be used.

Once an expression clone has been prepared, plasmid DNA for transfection may be prepared. Plasmid DNA for transfection into eukaryotic cells must be very clean and free from phenol and sodium chloride. Contaminants will kill the cells, and salt will interfere with lipid complexing, decreasing transfection efficiency. Plasmid DNA can be isolated using the S.N.A.P.™ MidiPrep Kit (Invitrogen Corporation, Carlsbad, Calif. Catalog no. K1910-01) or CsCl gradient centrifugation.

For established cell lines (e.g., COS, HeLa), consult original references or the supplier of the cell line for the optimal method of transfection. It is recommended that the protocol developed for individual cell lines be followed. Factors that may influence transfection efficiencies include medium requirements, when to pass the cells, and at what dilution to split the cells. Further information is provided in Current Protocols in Molecular Biology (Ausubel, F. M., et al., Current Protocols in Molecular Biology, Greene Publishing Associates and Wiley-Interscience, New York (1994)).

Methods for transfection include calcium phosphate (Chen, C., and Okayama, H., Mol. Cell. Biol. 7:2745-2752 (1987); Wigler, M., et al., Cell 11:223-232 (1977)), lipid-mediated (Felgner, P. L., et al., Proc. West. Pharmacol. Soc. 32:115-121 (1989); Felgner, P. L., and Ringold, G. M., Nature 337:387-388 (1989)) and electroporation (Chu, G., et al., Nucleic Acids Res. 15:1311-1326 (1987); Shigekawa, K., and Dower, W. J., BioTechniques 6:742-751 (1988)). If a cationic lipid-based reagent for transfection is used, one suitable reagent is Lipofectamine™ 2000 Reagent available from Invitrogen Corporation, Carlsbad, Calif. (Catalog no. 11668-027). Other suitable transfection reagents may also be used.

pcDNA™6.2/V5-GW/p64^{TAG} or pcDNA™6.2/GFP-GW/p64^{TAG} is provided as a positive control vector for mammalian cell transfection and expression and may be used to optimize recombinant protein expression levels in a particular cell line. These vectors allow expression of native or C-terminally-tagged recombinant human c-myc (p64) protein that may be detected by Western blot. If using these vectors as expression controls, be aware that the p64 protein is naturally associated with nucleolar structures and requires ionic detergents (RIPA or SDS gel loading buffer) to adequately solubilize in total cell lysates prior to western blot analysis.

To propagate and maintain each of the control plasmids resuspend the vector in 10 μl sterile water to prepare a 1 μg/μl stock solution and use the stock solution to transform a recA, endA E. coli strain like TOP10, DH5α™, or equivalent. Transformants can be selected on LB agar plates containing 100 μg/ml ampicillin or Low Salt LB agar plates containing 100 μg/ml Blasticidin. A glycerol stock of a transformant containing plasmid can be prepared for long-term storage.

The methods described herein (e.g., the Tag-on-Demand™ System) can be used to express both native and C-terminally-tagged recombinant polypeptide in mammalian cells from the same pcDNA™6.2/V5-DEST or pcDNA™6.2/GFP-DEST expression construct. To use the Tag-on-Demand™ System, add the Tag-on-Demand™ Suppressor Supernatant to mammalian cells at a specified time

In some embodiments, particularly those in which an adenovirus is used to transduce a host cell in order to express a suppressor tRNA, the host cell may be transduced with the adenovirus followed immediately by transfection with the expression construct containing a sequence of interest encoding a polypeptide of interest. Embodiments of this type may be used to quickly screen for expression (or localization, if possible) of a recombinant polypeptide or to screen for expression of a large number of polypeptides. Embodiments of this type will be discussed in greater detail in the following example.

In some embodiments, it may be desirable to generate a stable cell line comprising a nucleic acid molecule encoding a polypeptide of interest. In embodiments of this type, a nucleic acid molecule encoding a suppressor tRNA may be introduced into the stable cell line to produce fusion polypeptides comprising the polypeptide of interest fused to an additional polypeptide sequence (e.g., a tag sequence, etc.). For example, a stable cell line may be transduced with an adenoviral vector expressing one or more suppressor tRNAs (e.g., the Tag-On-Demand™ Suppressor Supernatant) to produce a C-terminally-tagged recombinant polypeptide.

In some embodiments (e.g., the Tag-on-Demand™ Suppressor Supernatant), nucleic acid molecules of the invention may be purified, titered, replication-incompetent, recombinant adenoviruses containing a human tRNA^{TAG} suppressor. Transduction of the adenovirus into mammalian cells facilitates transient stop suppression at the TAG codon in a gene of interest, enabling production of C-terminally-tagged recombinant polypeptide.

In some embodiments (e.g., the Tag-on-Demand™ Suppressor Supernatant), a nucleic acid molecules of the invention may be recombinant adenovirus that is deleted in the E1 region. Such an adenovirus is replication-incompetent in any mammalian cells that do not express the E1 proteins. Using such adenoviruses in 293 cells or in any cell line that expresses the adenovirus E1 gene (Graham, F. L., et al., J. Gen. Virol. 36:59-74 (1977); Kozarsky, K. F., and Wilson, J. M., Curr. Opin. Genet. Dev. 3:499-503 (1993); Krougliak, V., and Graham, F. L., Hum. Gene Ther. 6:1575-1586 (1995)) results in viral replication and will lead to rapid death of the target cell within 1-2 days after infection.

Using methods of the invention, fusion polypeptides may be expressed transiently or stably. To express a recombinant fusion polypeptide transiently, nucleic acid molecules encoding the fusion polypeptide of interest and encoding and nucleic acid molecules encoding a suppressor tRNA may be introduced into a host cell. One skilled in the art will appreciate that the sequences encoding the fusion polypeptide of interest and the suppressor tRNA may be on the same or different nucleic acid molecules. In embodiments where an adenovirus is used to express a suppressor tRNA, cells may be transduced with the adenovirus and then transfected with the expression construct (i.e. nucleic acid molecule encoding the fusion polypeptide).

To express a recombinant fusion polypeptide from a stable cell line, a stable cell line comprising a nucleic acid molecule encoding the fusion polypeptide of interest may be created using any standard technique or one or more of the techniques described herein (e.g., using lentiviral vectors or transfecting the mammalian cell line with the pcDNA™6.2/V5-DEST or pcDNA™6.2/GFP-DEST expression construct, etc.). A nucleic acid molecule encoding a suppressor tRNA (e.g., an adenovirus expressing a suppressor tRNA) may be introduced into the stable cell line to produce a fusion polypeptide.

In some embodiments, nucleic acid molecules of the invention (e.g., pcDNA™6.2/V5-DEST and pcDNA™6.2/GFP-DEST vectors) may contain one or more selectable markers that may be used to select for stable cell lines. In one embodiment, nucleic acid molecules of the invention may contain the Blasticidin resistance gene to allow selection of stable cell lines. Some methods of the invention may entail creating stable cell lines by transfecting a construct into a mammalian cell line of choice and selecting for foci using Blasticidin. Methods of creating stable cell lines may also comprise linearizing a nucleic acid molecule of the invention (e.g., pcDNA™6.2V5-DEST or pcDNA™6.2/GFP-DEST expression constructs) before transfecting them into a host cell. While linearizing the vector may not improve the efficiency of transfection, it increases the chances that the vector does not integrate in a way that disrupts elements necessary for expression in mammalian cells. Linearizing may comprise digesting the construct with a restriction enzyme that cuts at a unique site that is not located within a critical element or within the gene of interest.

In some embodiments, methods of generating a stable cell line expressing a polypeptide of interest may comprise determining the minimum concentration of Blasticidin required to kill the untransfected host cell line by performing a kill curve experiment using any one of the protocols described herein. Typically, concentrations ranging from 2.5 to 10 μg/ml Blasticidin are sufficient to kill most untransfected mammalian cell lines.

Once the appropriate Blasticidin concentration to use for selection has been determined, a stable cell line expressing a fusion polypeptide of interest (e.g., a pcDNA™6.2/V5-DEST or pcDNA™6.2/GFP-DEST construct) can be generated. Methods of creating a stable cell line may comprise transfecting a mammalian cell line of interest with a nucleic acid molecule of the invention (e.g., a pcDNA™6.2/V5-DEST or pcDNA™6.2/GFP-DEST construct) using any transfection method of choice and selecting a stable cell line. Selecting may comprise 24 hours after transfection, washing the cells and adding fresh growth medium. 48 hours after transfection, splitting the cells into fresh growth medium such that they are no more than 25% confluent. If the cells are too dense, the antibiotic will not kill the cells. Antibiotics work best on actively dividing cells. Selecting may further comprise incubating the cells at 37° C. for 2-3 hours until they have attached to the culture dish, removing the growth medium and replacing with fresh growth medium containing Blasticidin at the predetermined concentration required for the cell line. Methods of creating a stable cell line may also comprise feeding the cells with selective media every 3-4 days until Blasticidin-resistant colonies can be identified. Pick at least 5 Blasticidin-resistant colonies and expand them to assay for recombinant polypeptide expression.

Methods of the invention may comprise detecting a fusion polypeptide of the invention. For example, V5 fusion polypeptides expressed from pcDNA™6.2/V5-DEST can be detected using Western blot, immunofluorescence, or a functional assay specific the polypeptide of interest. A time course of expression may be prepared to optimize expression of the recombinant polypeptide (e.g. 24, 48, 72 hours, etc.). Anti-V5 Antibodies are available from Invitrogen Corporation, Carlsbad, Calif. and can be used to detect V5-tagged recombinant fusion polypeptides: For Western blot analysis, the Anti-V5-Horseradish Peroxidase (HRP) Antibody or the Anti-V5-Alkaline Phosphatase (AP) Antibody may be used for detection. For immunofluorescence, the Anti-V5-Fluorescein Isothiocyanate (FITC) Antibody can be used for detection.

Methods of detecting a fusion polypeptide may comprise performing a Western blot. Such a method may comprise preparing a cell lysate from transfected cells. Any suitable protocol for preparing a cell lysate known to those skilled in the art may be used. Preparing a cell lysate may comprise washing cell monolayers (e.g., ˜5×10⁵ to 1×10⁶ cells may be washed once with Phosphate-Buffered Saline, PBS, Invitrogen Corporation, Carlsbad, Calif., Catalog no. 10010-023). Preparing a cell lysate may further comprise scraping cells into a buffer and centrifuging the cells. For example, cells may be scraped into 1 ml PBS and cells may be centrifuged at 1500×g for 5 minutes to form a cell pellet. Methods of preparing a cell lysate may comprise re-suspending a cell pellet in a lysis buffer. For example, cells may be re-suspended in 50 μl Cell Lysis Buffer (e.g., 50 mM Tris, pH 7.8, 150 mM NaCl, 1% Nonidet P-40). Other cell lysis buffers known to those skilled in the art are also suitable. Re-suspending may comprise mixing (e.g., vortexing) the cell pellet in the lysis buffer to form a cell suspension and incubating the cell suspension (fore example, at 37° C. for 10 minutes) under conditions suitable to lyse the cells. Cells may be lysed at room temperature or on ice if degradation of polypeptide is a potential problem. Methods of preparing a cell lysate may further comprise centrifuging the cell lysate, for example, at 10,000×g for 10 minutes at +4° C. to pellet nuclei and transferring the supernatant to a fresh tube.

Lysates prepared according to the invention may be further analyzed using techniques well known in the art, for example, lysates may be assayed for protein concentration. Those skilled in the art will appreciate that protein assays utilizing Coomassie Blue or other dyes should not be used if the lysis buffer comprises NP-40 since NP-40 interferes with the binding of the dye with the protein.

Methods of performing a Western blot may comprise mixing an aliquot of a cell lysate with an SDS-PAGE. For example, SDS-PAGE sample buffer can be added to cell lysate to from a mixture and the mixture may be boiled, for example, for 5 minutes. An amount of the mixture comprising about 20 μg of protein may be loaded onto an SDS-PAGE gel and electrophoresed. One skilled in the art can select the appropriate concentration of acrylamide to be used to prepare the gel based upon the expected size of the fusion polypeptide.

One skilled in the art will recognize that a C-terminal tag containing the attB2 site and the V5 epitope will add approximately 4 kDa to the polypeptide of interest. Fusion polypeptides of the invention may also comprise additional amino acids located between the polypeptide of interest and the additional polypeptide sequence (e.g., a tag sequence such as the V5 epitope).

In some embodiments, methods of the invention may comprise detecting the presence of a fusion polypeptide comprising all or a portion of the p64 polypeptide. Methods of this type (e.g., fusion polypeptides expressed from the pcDNA™6.2/V5-GW/p64TAG control), may utilize any suitable detection means, for example, any of the Anti-V5 Antibodies and/or anti-myc antibodies discussed above. Methods of preparing a cell lysate from a cell expressing a fusion polypeptide comprising all or a portion of p64 may comprise the use of harsher extraction conditions since procedures using NP-40 lysis are not effective in releasing p64 protein. Since p64 is localized in the nucleoli, harsher lysis procedures using RIPA or SDS-PAGE sample buffer are required to adequately solubilize p64 in total cell lysates. Methods of this type may comprise washing cell monolayers, for example, once with Phosphate-Buffered Saline (PBS, Invitrogen Corporation, Carlsbad, Calif. Catalog no. 10010-023). Methods may further comprise add 1× SDS-PAGE Sample Buffer to each well containing cells. 1× SDS-PAGE buffer can be prepared by mixing 2.5 ml 0.5 M Tris-HCl, pH 6.8, 2 ml of glycerol (100%), 0.4 ml of β-mercaptoethanol, 0.02 g Bromophenol Blue, 0.4 g SDS and enough sterile water to bring the volume to 20 ml. For a 24-well plate, use 100 μl of 1× SDS-PAGE Sample Buffer per well. Methods may further comprise removing the cells from the plate, for example, a pipette tip may be used to loosen lysed cells from plate. Lysed cells may be transferred to a 1.5 ml microcentrifuge tube. Lysates prepared according to this method are typically viscous. Methods may further comprise heating samples, for example, at 70° C. for 10 minutes and mixing samples, for example, by vortexing every few minutes and briefly centrifuging the sample.

Methods may further comprise loading an aliquot of the cell lysate, for example, 5 μl of cell lysate, onto an SDS-PAGE gel and electrophoresing. One skilled in the art will appreciate that the V5-tagged p64^{TAG} protein has a molecular weight of approximately 53 kDa.

To detect the polypeptides expressed from as cycle-3 GFP fusion polypeptides from pcDNA™6.2/GFP-DEST, fluorescence, Western blot analysis, or a functional assay specific for the polypeptide of interest may be used. A time course may be prepared to optimize expression of the recombinant polypeptide (e.g. 24, 48, 72 hours, etc.). Any suitable technique, including those discussed herein, may be used to evaluate expression.

Cycle-3 GFP fusion polypeptides may be detected in vivo using fluorescence microscopy. The CMV promoter used to control expression of the cycle-3 GFP fusion polypeptide from pcDNA™6.2/GFP-DEST is a strong promoter and typically cycle-3 GFP fluorescence may be detected about 24 hours after transfection or transduction.

Methods of the invention may comprise methods of detecting fluorescent cells. In the practice of such methods, it is important to pick the best filter set to optimize detection. The primary excitation peak of cycle-3 GFP is at 395 nm. There is a secondary excitation peak at 478 nm. Excitation at either of these wavelengths yields a fluorescent emission peak with a maximum at 507 nm. Note that the quantum yield can vary as much as 5- to 10-fold depending on the wavelength of light that is used to excite the GFP fluorophore.

Use of the best filter set will insure that the optimal regions of the cycle-3 GFP spectra are excited and passed. Suitable filter sets include those designed to detect fluorescence from wild-type GFP (e.g., Omega Optical XF76 filter; see www.omegafilters.com). FITC filter sets may be used to detect cycle-3 GFP fluorescence, but note that these are not optimal and fluorescent signal may be weaker. For example, a FITC filter set may excite cycle-3 GFP with light from 460 to 490 nm, covering the secondary excitation peak and pass light from 515 to 550 nm. A set of this type may allow detection of most but not all of the cycle-3 GFP fluorescence.

Most tissue culture media fluoresce because of the presence of riboflavin (Zylka, M. J., and Schnapp, B. J., BioTechniques 21:220-226 (1996)) and may interfere with detection of cycle-3 GFP fluorescence. Medium can be removed and replaced with Phosphate-Buffered Saline (PBS, Invitrogen Corporation, Carlsbad, Calif., Catalog no. 10010-023) during the assay to alleviate this problem. If cells will be cultured further after assaying, remove the PBS and replace with fresh growth medium prior to re-incubation.

To detect expression of a cycle-3 GFP fusion polypeptide by Western blot, an antibody to the polypeptide of interest or an antibody to cycle-3 GFP may be used. GFP Antiserum is available separately from Invitrogen Corporation, Carlsbad, Calif. (Catalog no. R970-01) for detection. The GFP Antiserum is a purified, polyclonal rabbit antiserum raised against recombinant cycle-3 GFP, and can detect both cycle-3 GFP and wild-type GFP protein.

The C-terminal tag containing the attB2 site and cycle-3 GFP will add approximately 28.3 kDa to the size of the fusion polypeptide. Fusion polypeptides of the invention may further comprise additional amino acids located between the polypeptide of interest and cycle-3 GFP.

### Example 16

In some embodiments, the present invention provides materials and methods for the expression of fusion polypeptides. In one aspect, the same nucleic acid molecule is used to express a polypeptide of interest and a fusion polypeptide comprising the polypeptide of interest. In some aspects, this is accomplished by introducing into a host cell a nucleic acid molecule encoding a fusion polypeptide comprising a polypeptide of interest in the same reading frame as an additional polypeptide sequence. Typically, the nucleic acid molecule encoding the fusion polypeptide may comprise one or more stop codons, one of which may be located between the portion of the nucleic acid sequence encoding the polypeptide of interest and the portion of the nucleic acid sequence encoding the additional polypeptide sequence. In the presence of a nucleic acid molecule expressing one or more nucleic acid sequences encoding suppressor tRNA molecules, the stop codon between the two polypeptide sequences is suppressed and a fusion polypeptide is expressed.

Thus, in one aspect, the present invention comprises nucleic acid molecules (and/or compositions comprising such molecules) from which tRNA molecules (e.g., suppressor tRNA molecules) can be expressed. Nucleic acid molecules from which tRNA molecules can be expressed may be any type nucleic acid molecule known to those skilled in the art, for example, plasmids, linear nucleic acid molecules, viruses and the like. In a particular embodiment, the present invention provides a virus (e.g., an adenovirus, a lentivirus, a baculovirus etc.) from which a tRNA molecule may be expressed. In a specific embodiment, the present invention provides an adenovirus from which one or more tRNA molecule may be expressed.

In one embodiment, the present invention provides an adenovirus that expresses one or more suppressor tRNA molecules. One non-limiting example of such an adenovirus can be found in the Tag-On-Demand™ System commercially available from Invitrogen Corporation, Carlsbad, Calif. catalog number K400-01. Methods of the invention may employ an adenoviral-based stop suppression technology to allow expression of an untagged (i.e. native) or C-terminally-tagged recombinant polypeptide of interest in host cells from a single expression vector. In some embodiments, nucleic acid molecules of the invention may include Tag-On-Demand™ GATEWAY® vectors and/or other vectors, which may be used to generate an expression construct.

In one aspect, materials and methods of the present invention may be used to facilitate transient expression of a C-terminally-tagged recombinant polypeptide of interest in host cells (e.g., mammalian cells). Materials and methods of the invention may be used to provide a means to easily detect the expression or localization of a recombinant polypeptide(s) for which there is no specific antibody available. This may be useful, for example, in that once tagged recombinant polypeptide expression is verified, native polypeptide expression experiments may be performed with the same construct.

In some aspects, the present invention uses adenovirus as a delivery vehicle, enabling efficient delivery of suppressor tRNAs to a large variety of host cell types (e.g., mammalian cell types). Typically, in methods of the invention, suppressor tRNAs may be delivered transiently to cells to minimize toxicity.

In some aspects of the invention, methods of the invention may be used for high-throughput applications including rapid screening of a large number of genes for expression in a particular cell type.

In some embodiments, materials and methods of the invention may be used to transiently express C-terminally-tagged and native recombinant polypeptides in mammalian cells from a single expression construct. Suppressor tRNAs that function in mammalian cells have been described (see Capone, et al. (1985) EMBO J. 4, 213-221).

In one aspect, the present invention provides nucleic acid molecules (e.g., mammalian expression vectors) into which the a nucleic acid sequence encoding a polypeptide of interest will be cloned. Preferably, a nucleic acid sequence encoding a polypeptide of interest may be cloned into a nucleic acid molecule of the invention (e.g pcDNA™6.2/V5-DEST or pcDNA™6.2/GFP-DEST) in a configuration that is compatible with expression of C-terminally-tagged recombinant polypeptide by suppression of one or more stop codons.

In another aspect, the present invention provides nucleic acid molecules (e.g., replication-incompetent adenoviruses) comprising a nucleic acid sequence from which a suppressor tRNA can be expressed (e.g., the human tRNA^{ser} suppressor gene). In some embodiments, a suppressor tRNA may be a tRNA mutated to recognize one or more stop codons, for example, the TAG (amber stop) codon, and decode it as a serine. Nucleic acid molecules according to this aspect of the invention may be introduced into host cells to provide a transient source of the tRNA^{ser} suppressor. If the expression construct encoding a gene of interest with a TAG stop codon is present in the host cells, the stop codon will be translated as serine, allowing translation to continue through any downstream reading frame (i.e. C-terminal tag). This results in production of a C-terminally-tagged fusion polypeptide.

In one aspect, a nucleic acid molecule from which a suppressor tRNA molecule may be expressed may be a recombinant adenovirus and may be constructed as follows. A vector containing the gene encoding the tRNA^{ser} gene with its native promoter and terminator may be obtained, for example, from Dr. Uttam RajBhandary at the Massachusetts Institute of Technology. This tRNA^{ser} gene has been mutated such that the anticodon recognizes the TAG (amber) stop codon, and is referred to as the tRNA^{ser} suppressor gene (see Capone, et al. (1985)). The tRNA^{ser} suppressor gene may be PCR amplified and TOPO® Cloned into the pENTR/D-TOPO® vector available from Invitrogen Corporation, Carlsbad, Calif. (Catalog no. K2400-20) to generate a GATEWAY® entry clone. Using the entry clone above and a multimerization procedure described in Buvoli et. al., 2000 (Buvoli, et al. (2000) Mol. Cell. Biol. 20, 3116-3124), a GATEWAY® entry clone containing 8 tandem copies of the tRNA^{ser} suppressor gene can be generated. One such entry clone has been constructed and is named pENTR™-tRNA8^{TAG}. The pENTR™-tRNA8^{TAG} entry clone was recombined with Invitrogen's pAd/PL-DEST™ destination vector (Catalog no. V494-20) using the GATEWAY® LR recombination reaction to generate the adenoviral expression clone, pAd/GW-tRNA8^{TAG}. The pAd/GW-tRNA8^{TAG} expression construct was used in Invitrogen's ViraPower™ Adenoviral Expression System (Catalog no. K4940-00) to produce recombinant adenovirus, which was CsCl-purified and titered to generate the Tag-On-Demand™ Suppressor Supernatant.

In some embodiments, a nucleic acid molecule expressing a suppressor tRNA molecule may be a recombinant adenovirus, which may be used, for example, to deliver the tRNA^{ser} suppressor to host cells (e.g., mammalian cells). Although adenovirus has a very broad tropism and can be used to deliver the tRNA^{ser} suppressor to a large variety of host cell lines and cell types, materials an methods of the invention are not limited to those cells that can be transduced with adenovirus. Thus, materials and methods of the invention may be used with any host cell line or type known to those skilled in the art.

In the practice of the invention, nucleic acid molecules expressing one or more suppressor tRNA molecules may be introduced into host cells. When such nucleic acid molecules are introduced into host cells, they may be introduced into from about 25% to about 100% of the cell population, or from about 25% to about 90%, from about 25% to about 80%, from about 25% to about 70%, from about 25% to about 60%, from about 25% to about 50%, from about 50% to about 100%, from about 60% to about 100%, from about 70% to about 100%, from about 80% to about 100%, from about 90% to about 100%, from about 50% to about 90%, from about 50%, to about 80%, from about 50% to about 75%, from about 50% to about 70%, or from about 50% to about 60%. In some embodiments, nucleic acid molecules expressing one or more suppressor tRNAs may be adenoviruses and may transduce mammalian cells with extremely high efficiency, resulting in delivery of the tRNA^{ser} suppressor to nearly 100% of mammalian cells.

In some embodiments, nucleic acid molecules expressing suppressor tRNAs of the invention may not integrate into the host genome and expression of the suppressor tRNA may be transient and only persist for as long as the nucleic acid molecule (e.g., viral genome) is present (typically 7-8 days after transduction). Typically, once an nucleic acid molecule expressing a suppressor tRNA is introduced into host cells, the suppressor tRNA is expressed within 24 hours.

In some embodiments, a nucleic acid molecule expressing a suppressor tRNA molecule may be a virus (e.g., adenovirus). As is known in the art, viruses may possess the ability to bind to one or more receptors that may be present on a cell surface. For example, adenovirus enters target cells by binding to the Coxsackie/Adenovirus Receptor (CAR) (see, Bergelson, et al. (1997) Science 275, 1320-1323) and internalizing via integrin-mediated endocytosis (see, Russell, W. C. (2000) J. Gen. Virol. 81, 2573-2604). Once internalized, the recombinant adenovirus is actively transported to the nucleus, and begins to express the suppressor genes. Thus, when the nucleic acid molecule expressing a suppressor tRNA is an adenovirus, the host cell line should contain CAR. Most mammalian cell types express CAR, but levels vary. Depending on the amount of the CAR expressed in a specific target cell line, transduction efficiencies may vary when an adenovirus is used to express a suppressor tRNA. One skilled in the art will appreciate that other viruses may be used to express suppressor tRNAs in the practice of the invention, for example, vaccinia virus, herpes virus, adeno-associated virus, baculovirus, retroviruses (e.g., lentivirus), plant viruses (e.g., tobacco mosaic virus, cauliflower mosaic virus, etc.), negative stranded RNA viruses (e.g., Sendai virus, etc.), positive stranded RNA viruses (eg., alphaviruses, etc.). One skilled in the art can readily select an appropriate virus to infect any desired type of target cell based on the known tropisms of specific viruses for specific cell types.

In some embodiments, nucleic acid molecules expressing a suppressor tRNA of the invention may be adenoviruses. Adenoviruses for use in this aspect of the invention may have one or more deletions in the adenoviral genome compared to a wild-type adenoviral genome (e.g., Ad2, Ad5, etc.). For example, an adenovirus for use in the invention may be deleted in the E1 and/or E3 regions. In some embodiments, the entire E1 and E3 regions may be deleted. Such viruses may be replication-incompetent when transduced into mammalian cells that do not express the E1a or E1b proteins (see, Graham, et al. (1977) J. Gen. Virol. 36, 59-74; Kozarsky and Wilson (1993) Curr. Opin. Genet. Dev. 3, 499-503; and Krougliak and Graham (1995) Hum. Gene Ther. 6, 1575-1586).

As is known in the art, adenovirus does not integrate into the host genome upon transduction. Because the virus is replication-incompetent, the presence of the viral genome is transient and will eventually be diluted out as cell division occurs. Because the adenovirus is present transiently in mammalian cells, the production of C-terminally-tagged polypeptide resulting from stop suppression is also transient. As levels of adenovirus decrease, levels of C-terminally-tagged polypeptide produced decrease.

In some embodiments, viruses used to express suppressor tRNAs in accordance with the invention may be replication-incompetent in the cell type in which they express the suppressor tRNA. Viruses for use in this aspect of the invention may be screened for the presence of wild-type replication-competent viruses using techniques known in the art. For example, a population of adenovirus for use in the present invention may be screened for the presence of replication-competent adenovirus (RCA) contamination using a supernatant rescue assay (see, Dion, et al. (1996) J. Virol. Methods 56, 99-107) with a detection sensitivity of one wild-type RCA per 10⁹ recombinant adenovirus. In some embodiments, a viral preparation to be used to express one or more suppressor tRNA molecules in accordance with the methods of the invention may contain no detectable wild-type RCA.

In some embodiments, the present invention provides methods to express a C-terminally-tagged fusion polypeptide, comprising transducing a host cell with a virus expressing one or more suppressor tRNA molecules, transfecting the transduced cells with one or more nucleic acid molecules encoding all or a portion of a fusion polypeptide, and incubating the host cell under conditions sufficient to express a C-terminally tagged fusion polypeptide. A schematic representation of an embodiment of this type is provided in FIG. 62. In another embodiment, the present invention provides methods to express a C-terminally tagged fusion polypeptide, comprising transducing a stable cell line comprising a nucleic acid molecule encoding all or a portion of a fusion polypeptide with a virus expressing one or more suppressor tRNA molecules and incubating the transduced cell under conditions sufficient to express a C-terminally tagged fusion polypeptide. A schematic representation of an embodiment of this type is shown in FIG. 63.

Methods of the invention may entail the use of stocks of viruses, for example, viruses expressing one or more suppressor tRNA molecules. As will be appreciated by those skilled in the art, stocks of viruses may be stored at −80° C. In general, stocks stored under these conditions are stable for at least 6 months. If a viral stock has been stored at −80° C. for longer than 6 months, the tier of the stock may be determined using standard techniques as viral titers may decrease with long-term storage. Viral stocks should not be repeatedly thawed and re-frozen as viral titers can decrease with more than 3 freeze/thaw cycles.

One skilled in the art is aware that the handling of materials containing viruses should be performed following the applicable Federal and institutional guidelines for working with potentially hazardous organisms. For example, all manipulations should be performed within a certified biosafety cabinet, all media containing virus should be treated with bleach, all material that comes into contact with virus (e.g., pipettes, pipette tips, and other tissue culture supplies) should be treated with bleach or disposed of as biohazardous waste, and persons handling material containing virus should wear appropriate safety clothing (e.g., gloves, a laboratory coat, and safety glasses or goggles).

In some embodiments, methods of the invention may be used to create a nucleic acid molecule encoding a fusion polypeptide. According to one aspect of the invention, a nucleic acid molecule encoding a fusion polypeptide may be constructed by combining a first nucleic acid molecule having a first nucleic acid sequence encoding a polypeptide sequence (e.g., a polypeptide of interest) with a second nucleic acid molecule having a second nucleic acid sequence encoding an additional polypeptide sequence (e.g., a polypeptide tag sequence). A nucleic acid molecule encoding a polypeptide of interest should contain an ATG initiation codon in the context of a Kozak consensus sequence for proper initiation of translation in mammalian cells (Kozak, 1987; Kozak, 1991; Kozak, 1990). An example of a Kozak consensus sequence is (G/A)NNATGG, where the ATG initiation codon is underlined. Other sequences are possible, but the G or A at position −3 and G at position +4 are the most critical for function (shown in bold).

Typically, a nucleic acid molecule encoding a polypeptide of interest will contain a stop codon, for example, encoded by the nucleotides, TAG, TAA or TGA. One skilled in the art will appreciate that an appropriate suppressor tRNA (i.e., with an anti-codon that corresponds to the stop codon) must be used.

One skilled in the art will appreciate that, after joining of the first and second nucleic acid molecules to produce a nucleic acid molecule encoding a fusion polypeptide, the sequence encoding the polypeptide of interest must be in the same reading frame as the additional polypeptide sequence.

Second nucleic acid molecules encoding an additional polypeptide sequence will typically comprise one or more stop codons after the sequence encoding the additional polypeptide sequence. In general, the stop codon on the second nucleic acid molecule will be different from the stop codon on the first nucleic acid molecule.

A wide variety of nucleic acid molecules are suitable for use as second nucleic acid molecules in accordance with the present invention. Non-limiting examples of such nucleic acid molecules include vectors commercially available from Invitrogen Corporation, Carlsbad, Calif. Examples of such vectors are provide with their Invitrogen Corporation, Carlsbad, Calif. catalog number in parenthesis. Such vectors include, but are not limited to, pLenti4/V5-DEST™ (K4980-00), pLenti6/V5-DEST™ (K4950-00), pLenti6/UbC/V5-DEST™ (K4990-00), pLenti6/V5-D-TOPO® (K4960-00), and pAd/CMV/V5-DEST (K4930-00), which may be used for viral expression; pcDNA5/FRT/V5-His-TOPO® (K6020-01), pSecTag/FRT/V5-His-TOPO® (K6025-01), pEF5/FRT/V5-DEST™ (V6020-20), and pEF5/FRT/V5-D-TOPO® (K6035-01), which may be used for expression from a specific genomic locus using the Flp-In™ System; pcDNA™4/TO/myc-His (K1030-01), pGene/V5-His (K1060-01), which may be used for inducible expression; pcDNA™6.2/V5-DEST (K420-01), pcDNA™3.2/V5-DEST (12489-019), pcDNA™-DEST40 (12274-015), pcDNA6.2/V5-GW/D-TOPO® (K2460-20), pcDNA3.2/V5-GW/D-TOPO® (K2440-20), pcDNA3.1D/V5-His-TOPO® (K4900-01), pcDNA™3.1/V5-His-TOPO® (K4800-01), pcDNA™3.1/V5-His (V810-20), pcDNA™3.1/myc-His (V800-20), pcDNA™3.1(−)/myc-His (V855-20), pcDNA™4/V5-His (V861-20), pcDNA™4/myc-His (V863-20), pcDNA™6/V5-His (V220-20), and pcDNA™6/myc-His (V221-20), which may be used for constitutive expression from the CMV promoter; pEF6/V5-His-TOPO® (K9610-20), pEF1/V5-His (V920-20), pEF1/myc-His (V921-20), pEF4/V5-His (V941-20), pEF4/myc-His (V942-20), pEF6/V5-His (V961-20), and pEF6/myc-His (V962-20), which may be used for constitutive expression from the EF-1α promoter; pUB6/V5-His (V250-20), which may be used for constitutive expression from the UbC promoter; pSecTag2 (V900-20), and pSecTag2/Hygro (V910-20), which may be used for constitutive secreted expression; and pcDNA™6.2/GFP-DEST (K410-01), pcDNA™-DEST47 (12281-010), and pcDNA3.1/CT-GFP-TOPO® (K4820-01), which may be used for fusion to the GFP reporter gene.

A variety of factors may be optimized to produce fusion polypeptides according to the methods of the invention. Factors include, but are not limited to, characteristics of the host cell line; the health of the cells and experimental cell culture conditions; the transfection method used to introduce nucleic acid molecules into the host cell line; the transduction procedure used; and the amount of nucleic acid encoding a suppressor tRNA introduced into the host cells (e.g., multiplicity of infection when the nucleic acid molecule encoding a suppressor tRNA is a virus such as an adenovirus).

In some embodiments, a fusion protein of the invention may be expressed in any host cell type known to those skilled in the art. In some embodiments, a host cell line may be a mammalian host cell line. When an adenovirus is used in the practice of the invention to express one or more suppressor tRNA molecules, a host cell line preferably expresses one or more receptors allowing efficient transduction of the cell line by the adenovirus. An example of a suitable receptor is the Coxsackie/Adenovirus Receptor (CAR) (see, Bergelson, et al. (1997) Science 275, 1320-1323). Most mammalian cell types express CAR, but levels vary. One skilled in the art will appreciate that transduction efficiencies of cell lines will vary depending on the amount of the CAR expressed in a given cell line and can adjust either the multiplicity of infection and/or the cell line used as necessary for any particular application using routine experimentation.

In some embodiments, cells lines used in the practice of the present invention may not express viral proteins necessary for replication of a virus used to introduce suppressor tRNAs into the host cells. For example, when an adenovirus is sued to introduce suppressor tRNAs into host cells, the host cells may not express the adenovirus E1 proteins.

Typically, host cells used in the practice of the invention may be amenable to efficient transfection. For example, it may be possible to introduce a nucleic acid molecule encoding a fusion polypeptide invention into a high percentage of cells using standard techniques. For example, using lipid-mediated transfection (for example, with Lipofectamine 2000), it may be possible to introduce a nucleic acid molecule encoding a fusion polypeptide of the invention into from about 25% to about 100%, from about 25% to about 99%, from about 25% to about 95%, from about 25% to about 80%, from about 25% to about 70%, from about 25% to about 60%, from about 25% to about 50%, from about 25% to about 40%, from about 40% to about 95%, from about 50% to about 95%, from about 60% to about 90%, from about 75% to about 95%, or from about 80% to about 95% of the cells of a given sample of cells (e.g., the cells in a well of a tissue culture plate). Examples of suitable cell lines include, but are not limited to, COS-7, CHO-S, HeLa, HT1080, and BHK-21, primary rat hippocampal and cortical neurons.

In some embodiments, nucleic acid molecules encoding suppressor tRNAs for use in the present invention may be adenoviruses. Such adenoviruses may be deleted in the E1 region, rendering them replication-incompetent in any cells that do not express the E1 proteins. Typically methods of the invention are not performed in cells that express the adenovirus E1 protein (e.g., 293 cells or derivatives) as viral replication may occur in these cells, leading to rapid death of the target cell within 1-2 days after infection. In some instances it may be desirable to practice methods of the invention in cells expressing the adenovirus E1 protein.

One skilled in the art will appreciate that the health of the cells to be used in methods of the invention may affect the expression of fusion polypeptide of the invention expressed in these cells. In general, in methods of the invention, cells should be healthy (i.e. exhibit>95% viability) at the time of plating. Poor quality cell stock (e.g. cells consistently allowed to become overgrown or confluent before passaging, growth media allowed to become yellow before re-feeding) can negatively impact suppression efficiency and the amount of fusion polypeptide expressed. Generally, freshly prepared media may be used in the practice of methods of the invention.

Methods of the invention may entail introducing one or more nucleic acid molecules into one or more host cells. Any method of choice may be used to transfect nucleic acid molecules into cells. Suitable methods include, but are not limited to, calcium phosphate (see Chen and Okayama (1987); Wigler et al. (1977)), lipid-mediated (see, Felgner et al. (1989); Felgner and Ringold (1989)), and electroporation (see Chu, et al. (1987); Shigekawa and Dower (1988)). Suitable conditions (e.g., reagents, incubation conditions, etc.) for introducing nucleic acid molecules into any specific cell line may be determined by consulting published literature, consulting the provider of the cell line in question, and/or by routine experimentation. In some embodiments, methods of the invention may entail introducing one or more nucleic acid molecules into one or more cells using lipid-mediated transfection with a suitable lipid reagent (e.g., a lipid reagent from Invitrogen Corporation, Carlsbad, Calif. such as a cationic lipid-based reagent, Lipofectamine™ 2000 Reagent).

In some embodiments, methods of the invention may comprise introducing one or more nucleic acid molecules into one or more cells using Lipofectamine™ 2000 Reagent (see, Ciccarone, et al. (1999) Focus 21, 54-55) a cationic lipid-based formulation designed for transfection of nucleic acids into eukaryotic cells. Methods of this type may comprise forming a complex comprising nucleic acid molecules and Lipofectamine™ 2000 Reagent and contacting cells with the complexes in culture medium in the presence of serum. Such methods may not comprise removal of complexes or medium change or addition following transfection. Alternatively, methods may comprise removal of complexes or medium change or addition following transfection, for example, at 4-6 hours after contacting cells with the complexes.

In some embodiments, the present invention may include a method of screening for expression of a polypeptide comprising introducing into a host cell a nucleic acid molecule expressing a suppressor tRNA and a nucleic acid molecule encoding the polypeptide; and detecting the present of the polypeptide. In some embodiments such methods may be used to screen for expression or localization of the polypeptide or to screen for expression of a large number of genes. Such methods may involve the use of an adenovirus expressing a suppressor tRNA and may involve transducing a host cell with the adenovirus and transfecting a nucleic acid molecule encoding the polypeptide. Typically, transfection of the nucleic acid molecule is done as soon as practical after transduction with the adenoviruses. In some embodiments, the cells may be contacted with a solution comprising the adenovirus and then nucleic acid molecules (e.g., in complex with a transfection reagent) may be added to the solution comprising the adenovirus. Optionally, a nucleic acid molecule encoding a polypeptide of the invention may be introduced into a host cell prior to transduction of the host cell with an adenovirus expressing one or more suppressor tRNAs. One skilled in the art will appreciate that transducing a host cell with an adenovirus and simultaneously (i.e. as soon as practically possible) transfecting with plasmid encoding a polypeptide of the invention can increase plasmid-derived gene expression as well as reduce toxicity to the cell (see, Cotten, et al. (1992) Proc. Natl. Acad. Sci. USA 89, 6094-6098; Curiel, et al. (1991) Proc. Natl. Acad. Sci. USA 88, 8850-8854; Guy, et al. (1995) Mol. Biotechnol. 3, 237-248; Honda, et al. (1996) J. Virol. Methods 58, 41-51; and Merwin, et al. (1995) J. Immunol. Methods 186, 257-266).

In some embodiments, it may be desirable to create a stable cell line comprising a nucleic acid molecule encoding a polypeptide of the invention using standard techniques and to transduce the stable cell line with an adenovirus expressing one or more suppressor tRNAs.

In methods of the invention that comprise transducing a host cell with a virus expressing one or more suppressor trans (e.g., an adenovirus), cells may be transduced with any desired amount of virus. For example, cells may be transduced with virus at a multiplicity of infection (MOI) of from about 0.1 to about 500, from about 0.25 to about 500, from about 0.5 to about 500, from about 0.75 to about 500, from about 1 to about 500, from about 2 to about 500, from about 3 to about 500, from about 4 to about 500, from about 5 to about 5000, from about 10 to about 500, from about 25 to about 500, from about 50 to about 500, from about 75 to about 500, from about 100 to about 500, from about 200 to about 500, from about 300 to about 500, from about 400 to about 500, from about 1 to about 250, from about 1 to about 200, from about 1 to about 150, from about 1 to about 100, from about 1 to about 75, from about 1 to about 50, from about 1 to about 25, from about 1 to about 20, from about 1 to about 15, from about 1 to about 10, from about 1 to about 5, from about 10 to about 400, from about 10 to about 300, from about 10 to about 200, from about 10 to about 100, from about 10 to about 75, from about 10 to about 70, from about 10 to about 65, from about 10 to about 60 from about 10 to about 55, from about 10 to about 50, from about 10 to about 45, from about 10 to about 40, from about 10 to about 35, from about 10 to about 30, from about 10 to about 25, from about 10 to about 20, or from about 10 to about 15. Thus, cells may be transduced at an MOI of about 1, about 5, about 10, about 15, about 20, about 25, about 30, about 35, about 40, about 45, about 50 ,about 55, about 60, about 65, about 70, about 75, about 80, about 85, about 90, about 95, or about 100. MOI is defined as the number of virus particles per cell and generally correlates with expression. Depending on the cell line used and the nature of the gene of interest, MOI may be varied to optimize expression of a fusion polypeptide of the invention using routine experimentation.

As an example, for the cell lines tested (i.e. COS-7, CHO-S, HeLa, HT1080, BHK-21, and primary rat hippocampal and cortical neurons), transduction at an MOI of 50 followed by immediate transfection of the nucleic acid molecule encoding a fusion polypeptide of the invention generally results in 50-80% suppression. This means that 50-80% of the polypeptide expressed from the nucleic acid molecule encoding the fusion polypeptide is expressed as the fusion polypeptide. Note that 100% suppression cannot be achieved at any MOI. Some untagged polypeptide will always be expressed.

One of skill in the art will appreciate that the % suppression (i.e. suppression efficiency) achieved when cells are transduced at a particular MOI (e.g. MOI=50) can vary and is dependent on a number of factors including: the amount of CAR expressed in the mammalian cell; the nature of the gene being expressed; the health of the cells at the time of transduction; phenotypic changes to the cells resulting from stop codon suppression.

Depending on the suppression efficiency and consequently, the amount of fusion polypeptide expressed, the % suppression achieved can be optimized by varying the MOI using routine experimentation. It is important to note that while the % suppression achieved can be increased by increasing the MOI, doing so may increase the likelihood of phenotypic changes to the cells.

Expression of a suppressor tRNA in a host cell may result in phenotypic changes in the cell (e.g. toxicity) since ⅓ of the endogenous stop codons (i.e. all genes containing the stop codon recognized by the suppressor tRNA) can be suppressed. This leads to potential addition of extra amino acids to the C-termini of cellular proteins other than the fusion polypeptide of interest. In some embodiments, it may be desirable to optimize methods of the invention in order to minimize phenotypic effects in a particular cell line of interest.

In embodiments of the invention where one or more suppressor tRNAs are expressed from an adenovirus, the adenovirus may deliver a transient source of the suppressor tRNA to the target cell. When the adenovirus is replication-incompetent, it does not stably integrate into the genome of the target cell, and will be diluted out gradually as cell division occurs. This results in an overall decrease in suppressor tRNA expression over time. In some embodiments, it may be desirable to detect fusion polypeptide of the invention from about 1 hour to about 5 days, from about 1 hour to about 4 days, from about 1 hour to about 3 day, from about 1 hour to about 2 day, from about 1 hour to about 1 day, from about 1 hour to about 20 hours, from about 1 hour to about 16 hours, from about 1 hour to about 12 hours, from about 1 hour to about 8 hours, from about 1 hour to about 4 hours, from about 1 hour to about 3 hours, from about 1 hour to about 2 hours, from about 8 hours to about 72 hours, from about 8 hours to about 60 hours, from about 8 hours to about 48 hours, from about 8 hours to about 36 hours, from about 8 hours to about 24 hours from about 8 hours to about 20 hours, from about 8 hours to about 16 hours, or from about 8 hours to about 12 hours after transduction of the cells of interest. Thus fusion polypeptide may be detected at about 4 hours, about 8 hours, about 12 hours, about 16 hours, about 20 hours, about 24 hours, about 28 hours, about 32 hours, about 36 hours, about 40 hours, about 44 hours, about 48 hours, about 52 hours, about 56 hours, about 60 hours, about 64 hours, about 68 hours, about 72 hours, about 76 hours, about 80 hours, about 84 hours, about 88 hours, or at about 92 hours after transduction of the cells.

Methods of the invention may be practiced using any number of cells grown in any apparatus for that purpose known in the art (e.g., tissue culture plates, tissue culture flasks, roller bottles, bioreactors, etc.) In some embodiments, tissue culture plates may be used (e.g., 6-well, 24-well, or 96-well plates). For high-throughput applications, 96-well plates may be used. When cells are grown in tissue culture plates for use in the present invention, cells may be plated such that they are be 90% confluent at the time of transduction. As an example, the cells may be transduced with an adenovirus expressing a suppressor tRNA at an MOI of 50 and transfected with a plasmid encoding a fusion polypeptide of the invention. As discussed above, any suitable transfection protocol and/or reagents may be used. The amounts of nucleic acid molecule encoding a fusion polypeptide of the invention and transfection reagent may be adjusted to comport with the number of cells to be transfected using techniques well known in the art.

In a non-limiting example, COS-7 cells may be plated at, for example, 8×10⁴ COS-7 cells/per well of a 24-well plate and cultured overnight at 37° C. On the following day, the cells may be transduced (e.g., with an adenovirus expressing a suppressor tRNA), for example, using an MOI=50. It may be assumed that the number of cells has doubled during overnight culture so that the number of cells is 2×8×10⁴=1.6×10⁵ cells. If the titer of the viral stock is, for example, 1×10⁹ pfu/ml, the amount of viral stock to add to the cells may be calculated as follows: 50 pfu/cell×1.6×10⁵ cells=8×10⁶ pfu/1×10⁹ (pfu/ml)=0.008 ml=8 μl to add to each well.

In some embodiments, it may be desirable to include one or more controls in the methods of the invention. For examples, methods of the invention may comprise transducing a host cell with a virus expressing a suppressor tRNA, transfecting the cells with a control nucleic acid molecule (e.g., a nucleic acid molecule encoding a fusion polypeptide with a reporter activity), and detecting a reporter activity. For example, pcDNA™6.2/GFP-GW/p64^{TAG} may be used as a positive control for transduction, transfection, and expression. In this plasmid, the p64 protein (human c-myc) containing a TAG stop codon is cloned in frame with the cycle-3 GFP reporter gene (see (Chalfie, M., et al., Science 263:802-805 (1994); Crameri, A., et al., Nature Biotechnol. 14:315-319 (1996)). Including pcDNA™6.2/GFP-GW/p64^{TAG} plasmid when conducting transduction and transfection methods of the invention allows detecting a reporter gene activity (e.g., assaying for cycle-3 GFP expression using fluorescence microscopy or c-myc expression using Western blot analysis), and evaluating transfection and/or transduction conditions.

In one non-limiting example, methods of the invention may be used to express a fusion polypeptide of the invention. Methods of the invention may comprise seeding cells into a suitable tissue culture vessel at a suitable density (e.g., at a density such that the cells will be approximately 90% confluent at the time of transduction). Optionally, cells may be incubated (e.g., at 37° C. overnight) after seeding. When a 24-well tissue culture plate is used, cells may be seeded in 500 μl of complete medium. Methods of the invention may comprise, on the day of transduction, removing the growth medium from each well of cells and replacing with fresh growth medium (for a 24-well plate, 250 μl of medium may be used). Methods may further comprise contacting the cells with a nucleic acid molecule encoding a suppressor tRNA (e.g., transducing the cells with an adenovirus expressing a suppressor tRNA). When the nucleic acid molecule expressing a tRNA is a virus, any suitable MOI may be used (e.g., 50). Methods may further comprise returning the transduced cells to an incubator.

In embodiments where the host cell line is a stable cell line comprising a nucleic acid molecule encoding a fusion polypeptide of the invention, methods of the invention may comprise incubating cells (e.g., for 5-6 hours at 37° C.) after introduction of a nucleic acid molecule encoding a suppressor tRNA (e.g., after transduction with an adenovirus expressing a suppressor tRNA). Typically, cells are incubated for at least 5 hours as transduction efficiency will be decreased at shorter times. Longer incubation time is possible (e.g. overnight), but will not increase the transduction efficiency and may increase cell toxicity. Methods may further comprise removing the medium containing virus from the cells (e.g., after 5-6 hours), washing the cells (e.g., with 500 μl of fresh, complete growth medium in a 24-well plate), adding complete growth medium (e.g., 500 μl of fresh, complete growth medium in a 24-well plate) and incubating the cells under conditions sufficient to express a fusion polypeptide of the invention (e.g. at 37° C. in an incubator for a suitable period of time). Methods of the invention may further comprise detecting the fusion polypeptide.

In some embodiments, a nucleic acid molecule encoding a fusion polypeptide of the invention may be introduced into a host cell (e.g., after the cell has been transduced as described above). For example, after transduction, a suitable amount of a nucleic acid molecule encoding a fusion polypeptide of the invention may be mixed in a suitable medium. For example, for a well of a 24 well plate 500 ng of plasmid DNA may be dissolved in 50 μl of Opti-MEM® I Reduced Serum Medium without serum and a suitable amount of a transfection reagent (e.g., a cationic lipid transfection reagent) may be mixed with a suitable amount of a medium (e.g., for a well of a 24 well plate, 1.5 μl of Lipofectamine™ 2000 may be mixed in 50 μl of Opti-MEMO® I Reduced Serum Medium). Both mixtures (i.e., DNA:medium and reagent:medium) may be incubated, for example, for 5 minutes at room temperature. The two mixtures may be combined and incubated, for example, for 20 minutes at room temperature to allow the formation of nucleic acid:transfection reagent complexes (e.g., DNA-Lipofectamine™ 2000 Reagent complexes). Methods of the invention may comprise adding the complexes (e.g., DNA-Lipofectamine™ 2000 Reagent complexes) directly to the growth medium containing viruses used to transduce the host cells. Methods may comprise incubating the cells (for example, for 5-6 hours at 37° C.). Typically, cells are incubated for at least 5 hours as transduction efficiency will be decreased at shorter times. Longer incubation time is possible (e.g. overnight), but will not increase the transduction efficiency and may increase cell toxicity. Methods may further comprise removing the medium containing virus from the cells (e.g., after 5-6 hours), washing the cells (e.g., with 500 μl of fresh, complete growth medium in a 24-well plate), adding complete growth medium (e.g., 500 μl of fresh, complete growth medium in a 24-well plate) and incubating the cells under conditions sufficient to express a fusion polypeptide of the invention (e.g. at 37° C. in an incubator for a suitable period of time). Methods of the invention may further comprise detecting the fusion polypeptide.

One skilled in the art can readily adjust the volumes of the various reagents described above to transduce cells in different tissue culture formats (e.g., vary the amounts of cells and medium used) in proportion to the difference in surface area of the tissue culture plates used. For example, a 96-well plate may be used having a surface area per well of 0.3 cm², cells may be seeded in a volume of 100 μl and transduced in a volume of 50 μl; a 6-well plate may be used having a surface are per well of 10 cm², cells may be seeded in a volume of 2 ml and transduced in a volume of 1 ml.

As a non-limiting example, for methods of the invention using COS-7 cells, the following seeding densities and reagent quantities for transduction and transfection may be used in different tissue culture formats. Note that the suggested DNA quantities are for transfection using Lipofectamine™ 2000 Reagent.

| Condition | 6-well | 24-well | 96-well |
|---|---|---|---|
| Seeding density | 3 × 10⁵ cells | 8 × 10⁴ cells | 1 × 10⁴ cells |
| MOI = 50 | 3 × 10⁷ virus | 8 × 10⁶ virus | 1 × 10⁶ virus |
| Amount of plasmid DNA | 2 μg | 500 ng | 320 ng |
| per well | | | |
| Amount of Lipofectamine ™ | 6 μl | 1.5 μl | 1 μl |
| 2000 Reagent per well | | | |

In methods of the invention in which an adenovirus is used to express a suppressor tRNA in a host cell, one skilled in the art will recognize that such expression is transient. Accordingly, expression of a fusion polypeptide of the invention from a transiently transfected plasmid generally peaks within 24-48 hours following transfection. To obtain maximal levels of fusion polypeptide of the invention, cells may be harvested and assayed for fusion polypeptide of the invention expression between 24 and 48 hours post-transfection. Since expression conditions will vary depending on the nature of a particular fusion polypeptide of the invention and its half-life, conditions described above may be optimized using routine experimentation to obtain maximal levels of fusion polypeptide expression.

Methods of the invention may comprise detecting a fusion polypeptide of the invention. In some embodiments, detection may be by Western blot and/or immunofluorescence. In methods of this type, an antibody that specifically binds to the polypeptide of interest portion of the fusion protein may be used. One skilled in the art will appreciate that this allows detection of fused and un-fused forms of the polypeptide of interest. Alternatively, an antibody that specifically bind to the additional polypeptide sequences of the fusion polypeptide of the invention allows detection of only the fusion polypeptide and not the polypeptide of interest lacking the additional polypeptide sequences.

In some embodiments, additional polypeptide sequences in a fusion polypeptide of the invention may be a fluorescent polypeptide (e.g., the green fluorescent protein (GFP)). In embodiments of this type, it may be desirable to detect the fluorescence of the fluorescent polypeptide. In a specific embodiment, methods of the invention may comprise detecting GFP. GFP may be detected, for example, in vivo using fluorescence microscopy. An example of a fusion polypeptide of the invention is the GFP-tagged p64^{TAG} fusion protein expressed from the plasmid pcDNA™6.2/GFP-GW/p64^{TAG}. Since the GFP-tagged p64^{TAG} protein is expressed from the strong CMV promoter, fusion protein is generally detectable within 24 hours after transfection.

To detect fluorescent cells, suitable filter sets to optimize detection may be employed. The primary excitation peak of cycle-3 GFP is at 395 nm. There is a secondary excitation peak at 478 nm. Excitation at either of these wavelengths yields a fluorescent emission peak with a maximum at 507 nm. Note that the quantum yield can vary as much as 5- to 10-fold depending on the wavelength of light that is used to excite the GFP fluorophore.

Use of the best filter set will insure that the optimal regions of the cycle-3 GFP spectra are excited and passed (emitted). A filter set designed to detect fluorescence from wild-type GFP (e.g. Omega Optical XF76 Filter) may be used. Alternatively, FITC filter sets may be used to detect cycle-3 GFP fluorescence. One skilled in the art will appreciate that these filter sets are not optimal and fluorescent signal may be weaker. For example, a typical FITC filter set excites cycle-3 GFP with light from 460 to 490 nm, covering the secondary excitation peak. The filter set passes light from 515 to 550 nm, allowing detection of most but not all of the cycle-3 GFP fluorescence.

Most tissue culture media fluoresce because of the presence of riboflavin (see, Zylka, M. J., and Schnapp, B. J. (1996) BioTechniques 21, 220-226) and may interfere with detection of cycle-3 GFP fluorescence. To alleviate this problem, methods of the invention may comprise removing the growth medium and replacing the growth medium with Phosphate-Buffered Saline (PBS; Invitrogen Corporation, Carlsbad, Calif., Catalog no. 10010-023) before assaying for GFP fluorescence. If cells are being cultured further after assaying, methods of the invention may comprise removing the PBS and replacing with fresh growth medium prior to re-incubation.

In some embodiments, methods of the invention may comprise detecting a fusion polypeptide of the invention comprising a polypeptide sequence of the GFP by Western blotting. For example, GFP-tagged p64^{TAG} fusion polypeptide can be detected by Western analysis using the following antibodies available from Invitrogen Corporation, Carlsbad, Calif.: to detect both untagged and GFP-tagged p64^{TAG} protein, an antibody that specifically binds to the p64 portion of the fusion polypeptide (i.e., one of the Anti-myc Antibodies) can be used; to detect GFP-tagged p64^{TAG} protein only, an antibody that specifically binds to the GFP portion of the fusion polypeptide (e.g., the GFP Antiserum) may be used.

In methods of the invention that comprise detecting a fusion polypeptide of the invention by Western blotting, a lysate of host cells expressing the fusion polypeptide may be prepared. For example, a cell lysate to assay for native or GFP-tagged p64 protein may be prepared. One skilled in the art will appreciate that procedures using NP-40 lysis are not effective in releasing p64 protein. Since p64 is localized in the nucleoli, harsher lysis procedures using RIPA or SDS-PAGE sample buffer may be used to adequately solubilize p64 in total cell lysates. Methods of preparing a cell lysate to assay for p64 protein, may comprise washing cell monolayers (e.g., washing once with Phosphate-Buffered Saline Invitrogen Corporation, Carlsbad, Calif., PBS, Catalog no. 10010-023); adding a suitable lysis buffer (e.g., 1×SDS-PAGE Sample Buffer) to each well containing cells (e.g., for a 24-well plate, 100 μl of 1×SDS-PAGE Sample Buffer per well may be used); loosening lysed cells from the plate (e.g., e a pipette tip can be used to loosen lysed cells from plate); and transferring the cells to a centrifuge tube (e.g., for cells from one well of a 24-well late a 1.5 ml microcentrifuge tube). Lysates will be viscous.

Methods of preparing a lysate may further include heating samples at 70° C. for 10 minutes. Optionally, methods may include mixing (e.g. using a vortex mixer) one or more times and briefly centrifuging the sample. A lysate prepared by methods of the invention may be further processed or analyzed using techniques well known in the art. For example, an aliquot of the lysate (e.g., 5 μl of cell lysate) may be loaded onto an SDS-PAGE gel and electrophoresed. The GFP-tagged p64^{TAG} protein has a molecular weight of approximately 77.2 kDa.

One example of a suitable lysis buffer is 1× SDS-PAGE Sample Buffer, which may be prepared by combining the following reagents in the amounts indicated: 0.5 M Tris-HCl, pH 6.8 (2.5 ml); Glycerol (100%) (2 ml); β-mercaptoethanol (0.4 ml); Bromophenol Blue (0.02 g); SDS (0.4 g); and sterile water to a final volume of 20 ml. Aliquots of the buffer may be frozen at −20° C. until needed.

In one specific embodiment, the following ORFs were amplified to contain a TAG stop codon, and cloned into the pENTR/D-TOPO® Gateway® vector to generate entry clones. The entry clones were then transferred into the pcDNA™6.2/GFP-DEST vector using the Gateway® LR recombination reaction to create expression clones: 1) human CGI-130 (GenBank Accession # BC003357), which localizes to the cytoplasm; 2) human nuclear splicing factor(GenBank Accession # BC000997), which localizes in the nucleus; and 3) human c-myc (GenBank Accession # BC000141), which localizes with the nucleoli. COS-7 cells were transduced with the an adenovirus expressing suppressor tRNA molecules (i.e., Tag-On-Demand™ Suppressor Supernatant, Invitrogen Corporation, Carlsbad, Calif.) at an MOI of 50 followed by transfection with the pcDNA™6.2/GFP-DEST expression constructs using the procedure described above. Twenty-four hours post-transfection, GFP fluorescence was assayed using fluorescence microscopy. Fluorescent micrographs for each expression construct are shown in FIG. 64. For all three proteins above, methods of the invention result in expression of detectable levels of GFP-tagged recombinant protein as measured by GFP fluorescence by 24 hours post-transfection. Also, the GFP-tagged recombinant protein is correctly localized to the appropriate cellular organelle. The expression construct containing ORF3 (BC000141) is the same construct as the control pcDNA™6.2/GFP-GW/p64^{TAG} plasmid described above.

In some instances, cell toxicity may be observed when transduction and transfection are performed sequentially with a 5-6 hour incubation period after transduction. Although suppression may be clearly observed under these circumstances, the cells may appear unhealthy and may detach from the plate. This phenomenon is not due to either virus alone or transfection alone.

It has been demonstrated that adenovirus transduction performed simultaneously with plasmid transfection results in reduced toxicity and increased plasmid-derived gene expression (see Cotten et al., Proc Natl Acad Sci USA 89(13):6094-8, (1992); Curiel et al., Proc Natl Acad Sci USA 88(19):8850-4, (1991); Guy et al., Mol Biotechnol. 3(3):237-48, (1995); Honda et al., J Virol Methods 58(1-2):41-51, (1996); Merwin et al., J Immunol Methods 186(2):257-66, (1995); Zatloukal et al., Verh Dtsch Ges Pathol. 78:171-6, (1994)).

A series of experiments were performed to directly compare the method of sequential transduction-transfection with a simultaneous transduction/transfection method. In addition to being easier to perform, the simultaneous method resulted in cells that were clearly healthier (normal morphologies and proper adherence) with no evidence of toxicity (FIG. 66, right panels) as compared to the sequential method (left panels). As an added benefit, transfection efficiencies were higher making detection of fluorescent cells easier. In FIG. 66, 8×10⁴ COS-7 cells were plated in 24-well format and transfected/transduced as follows: Sequential Method (left panels): Cells were transduced with an adenovirus expressing a suppressor tRNA molecule (Ad-tRNA^{TAG}) at an MOI of 50 for 5 hours, media was replaced and cells were grown overnight. The following morning, cells were transfected with 0.5 μg pcDNA6.2/GFP-GW/p64^{TAG} using 1.5 μl Lipofectamine 2000 for 6 hours, media was replaced and cells grown overnight. GFP fluorescence and brightfield microscope photos were taken the following day. Simultaneous Method (right panels): Cells were transfected/transduced simultaneously. Adenovirus expressing a suppressor tRNA (Ad-tRNA^{TAG}) at an MOI of 50 was applied to cells and pre-formed DNA:Lipid complexes (0.5 μg DNA+1.5 μl Lipofectamine 2000) were added directly to the virus and cells for 5 hours. Media was replaced and GFP fluorescence and brightfield microscope photos were taken the following day.

A variety of lipid/DNA ratios were also evaluated using the simultaneous transduction/transfection method (FIG. 67). All lipid/DNA ratios tested resulted in healthy, normal looking cells. Western blotting revealed that all ratios tested gave stop suppression greater than 50%, even at MOI 25, with suppression levels ranging from 63% to 87% when simultaneous transduction/transfection was used (FIG. 67, upper panels). In FIG. 67, 8×10⁴ COS-7 cells were plated in 24-well format and transfected/transduced as described for Sequential and Simultaneous methods above. Various Lipid/DNA ratios were tested, as indicated. 24 hours post transduction/transfection, 5 μl of each total cell lysate was analyzed on 4-12% NuPage gel, MOPS running buffer, transferred to PVDF membrane and Western blot probed with anti-myc antibody. Percent suppression was determined by densitometry

Gene expression levels were noticeably higher with the simultaneous method and there was no MOI-dependent shut-down of gene expression (i.e. no MOI-dependent toxicity) which was visible with the sequential method (FIG. 67, compare upper western blot panels with lower panels).

Thus, methods of producing a fusion polypeptide according to the invention may comprise transducing a host cell with an adenovirus expressing a suppressor tRNA and introducing a nucleic acid molecule encoding a fusion polypeptide into the host cell wherein the host cell is contacted with the adenovirus and the nucleic acid molecule at the same time. Such a method may comprise seeding host cells, transducing host cells with an adenovirus and contacting host cells with one or more complexes comprising one or more nucleic acid molecules and one or more transfection reagents. Adenovirus may be used at any suitable MOI as discussed above (for example, about 50). Methods may comprise incubating cells in the presence of adenovirus and complexes for from about 10 minutes to about 48 hours, from about 10 minutes to about 36 hours, from about 10 minutes to about 24 hours, from about 10 minutes to about 20 hours, from about 10 minutes to about 16 hours, from about 10 minutes to about 12 hours, from about 10 minutes to about 8 hours from about 10 minutes to about 7 hours, from about 10 minutes to about 6 hours, from about 10 minutes to about 5 hours, from about 10 minutes to about 4 hours, from about 10 minutes to about 3 hours, from about 10 minutes to about 2 hours, from about 10 minutes to about 1 hour, from about 10 minutes to about 45 minutes, from about 10 minutes to about 30 minutes, from about 1 hour to about 48 hours, from about 1 hour to about 36 hours, from about 1 hour to about 24 hours, from about 1 hour to about 20 hours, from about 1 hour to about 16 hours, from about 1 hour to about 12 hours, from about 1 hour to about 8 hours, from about 1 hour to about 7 hours, from about 1 hour to about 6 hours, from about 1 hour to about 5 hours, from about 1 hour to about 4 hours, from about 1 hour to about 3 hours, from about 1 hour to about 2 hours, from about 2 hours to about 48 hours, from about 3 hours to about 48 hours, from about 4 hours to about 48 hours, from about 5 to about 48 hours, from about 6 hours to about 48 hours, from about 7 hours to about 48 hours, from about 8 hours to about 48 hours, from about 9 hours to about 48 hours, or from about 10 hours to about 48 hours. Thus, cells may be incubated in the presence of virus and nucleic acid molecule about 1 hour, about 2 hours, about 3 hours about 4 hours, about 5 hours, about 6 hours, about 7 hours, about 8 hours, about 9 hours, about 10 hours about 12 hours, about 16 hours, about 20 hours, about 24 hours, about 36 hours or about 48 hours.

Methods may further comprise removing the solution comprising virus and complexes; contacting the cells with a suitable medium (e.g., a complete medium); and incubating the cells under conditions sufficient to produce a fusion polypeptide of the invention. Methods may further comprise detecting the fusion polypeptide using any technique known to those skilled in the art (e.g., fluorescence microscopy, western blotting, etc). In some instances, toxicity may be observed at later time points and may be cell type specific. Cells should be closely monitored for evidence of toxicity if incubations are carried out for extended periods of time (e.g., longer than about 24-48 hours).

Suitable amounts of cells, media, virus, DNA, and transfection reagent (Lipofectamine 2000=LF2K) for various size tissue culture trays are as follows

| | 6-well | 24-well | 96-well |
|---|---|---|---|
| COS cells seeded per well | 3 × 10⁵ cells | 8 × 10⁴ cells | 1 × 10⁴ |
| media/well for culturing | 2 ml | 500 μl | 100 μl |
| MOI 50 | 3.0 × 10⁷ virus | 8 × 10⁶ virus | 1 × 10⁶ virus |
| MOI 25 | 1.5 × 10⁷ virus | 4 × 10⁶ virus | 5 × 10⁵ virus |
| media/well during tdx/tfx | 1 ml | 250 μl | 50 μl |
| Transfect DNA/well | 2 μg | 500 ng | 320 ng |
| LF2K/well | 6 μl | 1.5 μl | 1 μl |

Methods of the invention may comprise one or more incubation steps that may be performed in complete medium as described herein, unless otherwise indicated. Cell numbers are for COS-7 cells. Other cell types may require different cell numbers. Cells should be ˜90% confluent on the day of virus transduction. Assume that the number of cells double from the time of seeding to the time of transduction for the purpose of calculating MOI.

An example of a complete media is DMEM (high glucose) supplemented with FBS to a final concentration of 10%, L-glutamine to a final concentration of 4 mM, and MEM non-essential amino acids to a final concentration of 0.1 mM. These reagents are commercially available from, for example, Invitrogen Corporation, Carlsbad, Calif. (DMEM (high glucose) catalog no. 11960-044, FBS catalog no. 16000-044, L-glutamine (200 mM) catalog no. 25030-081, MEM non-essential amino acids (10 mM=100×) catalog no. 11140-050). Media should not be warmed in a water bath. Media should be allowed to come to room temperature in the dark.

As discussed above, in some embodiments, methods of the invention may comprise making a cell lysate. One suitable method for making a lysate entails removing media from the wells, adding a suitable lysis buffer (e.g., for a 24-well plate, 100 μl of 2× NuPAGE® LDS Sample Buffer (4×NuPAGE® LDS sample buffer is available from Invitrogen Corporation, Carlsbad, Calif., catalog no. NP0007 and can be diluted with water)) with 1/50^{th} volume of β-mercaptoethanol to each well; loosening the cells from the plate (e.g., using a pipette tip in a swirling motion to loosen lysed cells from plate); and transferring to a centrifuge tube (e.g., 1.5 ml eppendorf tube). Typically, lysates may be viscous. If it is too viscous, more 2×NuPage LDS Sample Buffer with β-mercaptoethanol can be added up to a total of 200 μl. Samples should be stored at −80° C. until conclusive western blotting has been completed.

Methods may further entail heating samples (e.g., at 70° C. for 10 minutes); mixing samples one or more times (e.g., vortexing and centrifugation throughout); loading an aliquot of the sample on an SDS-PAGE gel (e.g., loading 5 μl (per 100 μl harvested) on a 4-12% NuPage Bis-Tris gel, Invitrogen Corporation, Carlsbad, Calif., catalog no. NP0322BOX). Each gel may contain one or more controls molecular weight markers (e.g., 5 μl of Magic Mark, Invitrogen Corporation, Carlsbad, Calif., catalog no. LC5600, 10 μl of See Blue Plus 2, Invitrogen Corporation, Carlsbad, Calif. LC5925), and Western blot controls (e.g., 10 μl of Positope, Invitrogen Corporation, Carlsbad, Calif. R90050) heated at 70° C. Electrophoresis-may be performed using, for example, 1×NuPage MOPS SDS Sample Running Buffer (Invitrogen Corporation, Carlsbad, Calif., catalog no. NP0001). Add 500 μl of NuPage Antioxidant (Invitrogen Corporation, Carlsbad, Calif., catalog no. NP0005) to the sample running buffer in the “inner core.” Electrophoresis may be performed for a suitable period of time under suitable conditions (e.g., for approximately 50 minutes at 200 volts).

For Western blotting of gel, make up a suitable transfer buffer (e.g., 1×NuPage Transfer Buffer with 20% methanol, Invitrogen Corporation, Carlsbad, Calif. catalog no. NP0006). Add 1 ml of Antioxidant to 1 liter of 1×NuPage Transfer Buffer. Wet PVDF membranes (Invitrogen Corporation, Carlsbad, Calif. catalog no. LC2002) in methanol, rinse with H₂O, and then equilibrate in Transfer Buffer. Transfer to PVDF membrane for 90 minutes at 30 volts. Follow all procedures and recommendations in NuPage Bis-Tris gel package insert (Invitrogen Corporation, Carlsbad, Calif.).

Following transfer, wash membrane 2× with 20 ml of H₂O. Block membrane using a suitable blocking solution (e.g., that provided in the anti-mouse Western Breeze Chemiluminescent Kit, Invitrogen Corporation, Carlsbad, Calif., catalog no. WB7104). Blocking can be done for 30 minutes up to overnight. Dilute suitable antibody in an appropriate buffer (e.g., for detecting myc protein, anti-myc antibody (Invitrogen Corporation, Carlsbad, Calif., catalog nos. R95025, R95225, or 95325) can be diluted 1:5000 in PVDF primary antibody diluent. Incubate antibody solution with membrane, wash, and detect bound antibody using standard techniques suitable for the antibodies used (e.g., chemiluminescent detection, fluorogenic detection, radiolabel detection, etc.). Such techniques are well known to those skilled in the art.

When using myc protein that becomes tagged with GFP upon suppression of the stop codon between the coding region of the two proteins (e.g., as expressed from pcDNA6.2/GFP-GW/p64^{TAG}), un-tagged myc (no virus control) should band around Magic Marks 55 kDa and myc tagged with GFP should band around Magic Marks 80 kDa. Densitometry can be done to determine % shift from untagged myc to GFP tagged myc (i.e., percent suppression).

Other suitable lysis techniques may be used. For example, harvest cells from 24 well plate with 100 μl of 1×Tris-Glycine Sample Buffer (Invitrogen Corporation, Carlsbad, Calif., catalog no. LC2676) containing 1/50^{th} volume of β-mercaptoethanol to each well. Use a pipette tip in a swirling motion to loosen lysed cells from plate and transfer to a 1.5 ml eppendorf tube. Lysates will be viscous, this is normal. If it is too viscous, more 1×Tris Glycine Sample Buffer with β-mercaptoethanol can be added up to a total of 200 μl. Samples can be stored at 4° C. Heat samples at 100° C. for 10 minutes (with vortexing and centrifugation throughout) prior to loading 5 μl (per 100 μl harvested) on a 4-20% Tris Glycine gel (Invitrogen Corporation, Carlsbad, Calif., EC60252BOX). Western blot analysis may be performed as above or using other suitable techniques know to those skilled in the art.

Another suitable lysis technique is as follows. Harvest cells from 24 well plate with 100 μl of 1×RIPA lysis buffer containing Complete Protease Inhibitor Cocktail (Roche, catalog no. 1 697 498) 50× in H₂O) & Pepstatin (Roche, catalog no. 253 286) 1000× in EtOH). Use pipette tip in a swirling motion to loosen lysed cells from plate and transfer to a 1.5 ml eppendorf tube. Lysates will be viscous. If it is too viscous, more RIPA lysis buffer can be added up to 150 μl total. These lysates can be analyzed as above or using other techniques know to those skilled in the art. Bradford Protein assay can be conducted with this lysis buffer to quantitate total amount of protein loaded. Store samples at −80° C. until ready to use. Thaw at room temperature, and then keep on ice.

One suitable protocol for conducting the Bradford protein assay is as follows:

In a 96 well U-bottom flexible polyvinyl chloride plate (Falcon Cat. No. 35-3911)

Perform a 1:10 dilution of cell lysates (e.g., prepared as described above) directly in the wells (9 μL of H₂O and 1 μL of lysate).

Load 10 μL of BSA standard curve to the 96 well plate (1000 μg/ml serial diluted 1:2 down to 15.625 μg/ml)

Add 190 μL Bradford reagent to 10 μL of diluted lysates and standard curve (1:5 dilution of BioRad Protein Assay Solution, Bio-Rad Corporation, Hercules, Calif., catalog no 500-002, 1 ml Solution and 4 ml H₂O)

Read at endpoint wavelength 595 on plate reader and display Reduced numbers.

Use 4 parameter fit for Graph.

The methods described above may scaled up or down as appropriate for the number of cells to be used. In some embodiments, particularly those involving high-throughput applications, it may be desirable to analyze a large number of samples in a 96-well format. The protocol for 96-well late applications is the same as the 24 well format described previously with the following modifications.

Seed COS-7 cells at 1×10⁴ cells/well in 100 μl/ well in a 96-well plate.

Assume doubling of cells in 24 hour period to 2×10⁴ cells/well.

Transduction & Transfection are conducted in 50 μl/well volumes (100 μl total- 50 culture media, 50 complexes) for 5 hours. Complexes may be formed using 25 μl medium (e.g., 1×OPTIMEM) and 1 μl transfection reagent (e.g., Lipofectamine 2000) and 25 μl medium and 320 ng DNA incubated separately for 5 minutes at room temperature and then combined and incubated for 20 minutes at room temperature.

Cells may be harvested with 30-60 μl of Sample Buffer or 30-50 μl Lysis Buffer, depending upon viscosity.

Load 10 μl (per 30 μl harvested) of samples harvested with Sample Buffer on gel.

The most likely sources of low suppression efficiency include poor quality of cell stock at time of plating experiment (i.e., cells very confluent, media not pink) and the use of old media. Media should be freshly prepared for use in transduction/transfection.

In another specific example of methods of the invention, COS-7 cells were transduced with an adenovirus expressing suppressor tRNA molecules (i.e., the Tag-On-Demand™ Suppressor Supernatant) at various MOIs following the procedures described above and simultaneously transfected with the pcDNA™6.2/GFP-GW/p64^{TAG} plasmid using Lipofectamine 2000 Reagent and the procedure described above. Twenty-four hours post-transfection, cell lysates were prepared and analyzed by Western blot using the Anti-myc Antibody and the WesternBreezee® Chemiluminescent Anti-Mouse Kit (Catalog no. WB7104) to detect native and GFP-tagged p64^{TAG} (c-myc) protein. The results are shown in FIG. 68. In FIG. 68, Lane 1 contains MagicMark™ MW Standard, lane 2 contains untransfected COS-7 cells, lane 3 contains cells transduced at an MOI=0, lane 4 contains cells transduced at an MOI=50, lane 5 contains cells transduced at an MOI=100, lane 6 contains cells transduced at an MOI=200. GFP-tagged c-myc protein is produced and detectable by Western blot within 24 hours post-transfection. The % suppression achieved is >80% when transducing cells at an MOI≧50. In this experiment, increasing the MOI has little effect on the suppression efficiency. Maximal levels of GFP-tagged c-myc protein are produced using an MOI=50.

In another working example of methods of the invention, 96 of Invitrogen's Ultimate™ Human ORF Clones encoding 96 different kinases were transferred into the pcDNA™6.2/V5-DEST vector using the Gateway® LR recombination reaction to generate expression clones. The expression constructs were purified, and the plasmid DNA (ranging from 20 ng to 300 ng) was transfected using Lipofectamine™ 2000 Reagent into COS-7 cells (plated in 96-well format) that had been transduced with the Tag-On-Demand™ Suppressor Supernatant at an MOI of 50 following the procedure described above. Forty-eight hours post-transfection, cell lysates were prepared and analyzed by Western blot using the Anti-V5 Antibody (Invitrogen, Catalog no. R961-25) and the WesternBreeze® Chemiluminescent Anti-Mouse Kit (Catalog no. WB7104) to detect V5-tagged fusion polypeptide. Using this antibody, native polypeptide is not detected. V5-tagged fusion polypeptide is produced and detectable by Western blot within 48 hours post-transfection. The levels of V5-tagged fusion polypeptide produced vary widely from gene to gene. This is expected since transfection and expression conditions are not optimized for each gene and can vary depending on the nature of the gene of interest. In this working example, the amount of plasmid DNA transfected and the amount of cell lysate loaded on the polyacrylamide gel were not quantitated for each sample (i.e. transfection and expression conditions were not optimized). In addition, antibodies to each of the 96 different kinase proteins do not exist. This example demonstrates the utility of methods of the invention to quickly screen and analyze the expression of large numbers of recombinant proteins for which antibodies do not currently exist.

As discussed above, methods of the invention may be optimized using routine experimentation in order to produce a desired quantity of fusion polypeptide of the invention. A variety of factors may be considered when optimizing experimental conditions. For example, in some initial experiments, low expression of the desired fusion polypeptide may be observed. This may be due to any one or more or a number of reason such as 1) low suppression efficiency; 2) phenotypic effects observed; 3) poor transfection efficiency; and 4) improper timing of the assay (i.e., assayed too early or too late).

Low suppression efficiency may result in a reduced production of a desired fusion polypeptide and may be observed when the host cells used were not healthy and/or were not plated at the correct density. One skilled in the art can optimize this factor by ensuring that cells are healthy and >95% viable before plating and are plated at the proper density. Low suppression efficiency may be observed when the media used was not fresh. This factor can be optimized by preparing fresh media for use in the practice of the present invention. Low suppression efficiency may be observed if the host cells are transduced with too little virus (i.e. low MOI). One skilled in the at can optimize transduction by testing varying MOIs starting at about 50. Low suppression efficiency may be observed when host cells express low levels of CAR. One skilled in the art can optimize this factor by using a cell line that expresses suitable levels of CAR (e.g. COS-7, CHO, HeLa). Low suppression efficiency may be observed if host cells are not transduced for an optimum length of time. One skilled in the art can optimize this factor by transducing for various periods of time, for example, about 5-6 hours.

Phenotypic effects on host cells caused by methods of the invention may result in reduced production of a desired fusion polypeptide of the invention. Factors that may be optimized to mitigate phenotypic effects include the length of incubation after transduction and transfection. One skilled in the art can optimize this factor by assaying for fusion polypeptide at various times after transduction and transfection (e.g., 24-48 hours). Phenotypic effects may be observed if host cells used are sensitive to transduction and transfection procedure. One skilled in the art can optimize this factor by performing methods of the invention in a different host cell line and/or by making a stable cell line containing the nucleic acid molecule encoding the fusion polypeptide and subsequently introducing a nucleic acid molecule encoding a suppressor tRNA (e.g., transducing with a virus expressing a suppressor tRNA).

Poor transfection efficiency may result in a reduced production of a desired fusion polypeptide of the invention. One skilled in the art can readily optimize this factor by testing various transfection reagent to identify one that provides a high transfection efficiency for the cell line being used.

Reduced production of a fusion polypeptide of the invention may be observed when fusion polypeptide expression is assayed at a sub-optimal time (i.e., too early or too late). One skilled in the art can optimize this factor by assaying at various times to determine when optimum expression is observed (e.g., by conducting a time course of expression).

### Example 17

In some embodiments, the invention provides nucleic acid molecules comprising all or a portion of a viral genome that comprise transcriptional regulatory sequences (e.g., promoters, repressors, etc.). In one specific embodiment, the invention provides nucleic acid molecules comprising all or a portion of a viral genome (e.g., a retroviral genome) that comprise a repressor sequence. A repressor sequence may inhibit or prevent transcription of a nucleotide sequence to which it is operably linked.

A repressor sequence may bind or may be bound by one or more molecules (e.g., peptides, small molecules, etc.). In one embodiment, a repressor sequence may bind a protein (e.g., a repressor protein). One example of a repressor to which binds a repressor protein is the tetracycline operator to which binds the tetracycline repressor protein. In the absence of tetracycline, the repressor protein binds to the tetracycline operator and prevents or inhibits transcription of a nucleotide sequence to which it is operably linked. In the presence of tetracycline, the repressor protein binds tetracycline and no longer binds to the repressor sequence.

In some embodiments, a repressor sequence and a promoter sequence may be operably linked to a sequence of interest. In embodiments of this type, the repressor sequence may prevent transcription of the sequence of interest from the promoter under some conditions (e.g., when a repressor protein is bound to the repressor sequence) and not under other conditions (e.g., in the absence of repressor protein or under conditions in which the repressor protein is not bound to the repressor sequence).

In one embodiment of the invention, a nucleic acid molecule comprising all or a portion of a lentiviral genome may also comprise a repressor sequence (e.g., the tetracycline operator) and/or may comprise a nucleic acid sequence encoding a polypeptide that binds to a repressor (e.g., the tetracycline repressor protein). Embodiments of this type may be used to construct host cells and/or host cell lines comprising a nucleic acid sequence of interest operably linked to a repressor sequence. Optionally, such host cells and/or host cell lines may comprise a nucleic acid sequence encoding a polypeptide that binds to the repressor sequence. In a particular embodiment, the present invention encompasses host cells and/or host cell lines in which a sequence of interest is operably linked to a tetracycline repressor sequence and a promoter sequence and further comprise a nucleic acid sequence encoding the tetracycline repressor protein. Such host cell lines provide the ability to regulate the transcription of the sequence of interest, i.e., in the absence of tetracycline, the sequence of interest is not transcribed or is transcribed at an insignificant level while in the presence of tetracycline the sequence of interest is transcribed at a much higher level (i.e., transcription is induced by tetracycline).

Host cells and/or host cell lines according to the invention may be any type of cell (e.g., dividing or non-dividing cells) and may be isolated cells or may be within a larger organism. Methods of the invention allow controlled gene expression in tissue culture cells and whole organisms.

In some embodiments, the present invention provides a method of making a cell expressing a repressor protein and cells made by such methods. Methods may comprise introducing into a cell a nucleic acid molecule comprising all or a portion of a viral genome and encoding a repressor protein. Such methods may also comprise selecting for a cell stably expressing the repressor.

In some embodiments, the present invention comprises methods of expressing a sequence of interest comprising introducing into a host cell expressing a repressor protein, one or more nucleic acid molecules comprising a sequence of interest operably linked to a repressor and a promoter. In some embodiments, a nucleic acid molecule comprising a sequence of interest operably linked to a repressor and a promoter may comprise all or a portion of a viral genome (e.g., a lentiviral genome). Methods may further comprise incubating the cell under conditions in which the repressor protein does not bind to the repressor sequence. Such conditions may include incubation in the presence of a molecule that prevents the repressor protein from binding to the repressor sequence. For example, when the repressor sequence is the tetracycline operator and the repressor protein is TetR, such conditions may comprise incubating the cell in the presence of tetracycline.

In one particular embodiment, the present invention provides two nucleic acid molecules (e.g., plasmids, viral vectors etc.) that may be used in the practice of the invention. A first nucleic acid molecule comprises a repressor sequence and a promoter and may comprise a sequence of interest operably linked to the repressor and promoter. A first nucleic acid molecule may also comprise one or more recognition sequences (e.g., recombination sites, topoisomerase sites, restriction enzyme sites, etc.). One non-limiting example of a first nucleic acid molecule is the plasmid, pLenti4/TO/V5-DEST, which contains two copies of the tetracycline operator sequence (TO) within the CMV promoter (CMVTetO₂). A map of this vector is provided as FIG. 70A and the nucleotide sequence is provided in Table 31. This plasmid also contains two recombination sites that do not recombine with each other. A sequence of interest may be operably linked to the promoter and repressor using any technique known in the art. In one embodiment, a sequence of interest may be operably linked to the promoter and repressor by conducting a recombination reaction between a sequence of interest flanked by recombination sites and the nucleic acid molecule of the invention. For example, pLenti4/TO/V5-DEST (FIG. 70A) can be reacted with a sequence of interest flanked by attR1 and attR2 sites to operably link the sequence of interest to the CMV promoter and tetracycline operator in a LR-recombination reaction. The reaction places the sequence of interest downstream of CMVTetO₂ for regulated expression in the presence of the tetracycline repressor protein.

A second nucleic acid molecule of the invention may express one or more proteins that interact with repressor sequences. One non-limiting example of a repressor protein is the tetracycline repressor protein (TetR). One example of a suitable second nucleic acid molecule is the repressor plasmid pLenti6/TR, which expresses TetR. A map of this vector is provided as FIG. 69 and the nucleotide sequence is provided as Table 32. TetR binds the tetracycline operator sites in CMVTetO₂ promoter on the expression vector and blocks transcription from the promoter in the absence of inducer. When tetracycline inducer binds TetR, however, the latter dissociates from the promoter and transcription proceeds.

Methods of the of the invention may be use to regulate the expression of a sequence of interest in transformed dividing cells and in difficult-to-transfect growth-arrested primary cells. Methods of the invention may be used for transient or stable gene regulation. Induction of expression may be from about 2-fold to about 100-fold, from about 5-fold to about 100-fold, from about 10-fold to about 100-fold, from about 25-fold to about 100-fold, from about 50-fold to about 100, from about 75-fold to about 100-fold, from about 5-fold to about 5-fold to about 70-fold, from about 10-fold to about 70-fold, from about 25-fold to about 70-fold, from about 50-fold to about 70-fold, or from about 60-fold to 70-fold. Thus, gene expression may be induced about 5-fold, about 10-fold, about 20-fold, about 30-fold, about 40-fold, about 50-fold, about 60-fold, about 70-fold, about 80-fold, about 90-fold, or about 100-fold.

In some embodiments of the invention, the present invention may comprise a viral stock that may be used to transduce host cells. Stocks maybe at any suitable concentration of virus. For example, pLenti6/TR may be used to create a viral stock at about 1×10⁵ cfu/ml or greater, which may be used to stably transduce TetR into target cells in blasticidin-containing media. Cells transduced in this fashion will typically express TetR protein at a level detectable by Western blot.

In another aspect of the invention, the present invention provides nucleic acid molecules comprising a promoter sequence and a repressor sequence to which a sequence of interest may be operably linked. Such nucleic acid molecules may be used to create a viral stock. For example, recombinational cloning of the lacZ gene into pLenti4/TO/V5-DEST and packaging the resulting pLenti4/TO/V5-GW/lacZ vector may be used to produce a viral stock at 1×10⁵ cfu/ml or greater. Such a viral stock may be used to transduce a host cell and express a sequence of interest (e.g., the lacZ sequence).

In some embodiments, nucleic acid molecules comprising a promoter and repressor sequence operably linked to a sequence of interest and nucleic acid molecules comprising a sequence encoding a polypeptide that binds to the repressor sequence may be introduced into a host cell. In methods of this type, nucleic acid molecules may be introduced simultaneously or sequentially. Typically, once both types of nucleic acid molecule have been introduced into a host cell, expression of the sequence of interest will be inducible. For example, transient co-transduction of Lenti6/TR and Lenti4/TO/V5-GW/LacZ may show at least 20-fold induction in HT1080 cells. In some embodiments, after both types of nucleic acid molecule are introduced into a host cell, a stable cell line may be produced, for example, by selecting for cells expressing both the sequence of interest and the repressor protein. In one example, cell lines may be made that contain Lenti6/TR and Lenti4/TO/V5-GW/lacZ and such cells may show at least 20-fold induction.

One aspect of the present invention is the capability to regulate the expression of a sequence of interest in a non-dividing cell. In a specific embodiment, the present invention provides non-dividing host cells containing a sequence of interest, the expression of which is regulatable, for example, is inducible by the addition of tetracycline to the growth medium of the cell. The present invention contemplates compositions comprising such cells and further comprising one or more component selected from a group consisting of an inducer (e.g., tetracycline), a growth medium, and a buffer.

A nucleic acid molecule expressing the tetracycline repressor protein may be constructed using any technique known in the art. For example, a nucleic acid fragment containing the tetracycline repressor coding sequence can be cloned using any technique known in the art. The nucleotide sequence of a nucleic acid fragment containing the coding sequence for the tetracycline repressor is provided as Table 35. The 1.4 kb fragment also contains the β-globin intron. The 1.4 kb TetR-containing fragment was cloned into pLenti6/V5 (Invitrogen Corporation, Carlsbad, Calif.). A map of pLenti6/V5 is provided as FIG. 71 and the nucleotide sequence is provided as Table 33. The resulting plasmid, pLenti6/TR, was verified by restriction digest and sequence analyses. A map of pLenti6/TR is shown in FIG. 69. pLenti6/TR can be used to generate blasticidin resistant mammalian cells that stably express the tetracycline repressor, TetR.

Nucleic acid molecules comprising a promoter sequence and a repressor sequence can be constructed using any techniques known in the art. For example, pLenti4/TO/V5-DEST was created from pLenti3/V5-TREx (Invitrogen Corporation, Carlsbad, Calif.), by replacing the neomycin resistance gene of the latter with the zeocin resistance gene. pLenti3/V5-TREx contains the CMV promoter and Tet operators of pT-REx-DEST30 (Invitrogen Corporation, Carlsbad, Calif. catalog no. 12301016). A map of pLenti3/V5-TREx is provided as FIG. 72 and the nucleotide sequence is provided in Table 34.

pLenti3/V5/TREx was digested with SalI, filled in using Klenow and then digest with KpnI and the 5917 bp vector backbone was gel isolated. Next, pLenti4/V5-DEST (Invitrogen Corporation, Carlsbad, Calif. catalog nos. K498000 and V49810) was digested with Spel, Klenow filled-in, then digested with KpnI. A 2682 bp fragment of pLenti4/V5-DEST containing a GATEWAY™ Destination cassette, SV40 promoter and Zeocin resistance cassette, was gel isolated and ligated to the SalI-Klenow-KpnI processed pLenti3/V5-TREx. The ligation mixture was transformed into DB3.1 and selected on LB media containing Amp (100 μg/ml) and chloramphenicol (15 μg/ml). Colonies of the transformants were analyzed by restriction analysis. A map of pLenti4/TO/V5-DEST is shown in FIG. 70A. The GATEWAY™ Destination vector pLenti4/TO/V5-DEST contains the tet-regulated CMVTetO₂ T-REx promoter (consisting of CMV promoter and two tet operator sites). TetR protein binds the tetO sites to inhibit gene transcription; tetracycline relieves the transcription inhibition. pLenti4/TO/V5-DEST confers zeocin resistance and allows in-frame fusion of genes-of-interest to the V5 epitope tag.

pLentiTO/V5-GW/lacZ was generated by standard Gateway LxR reaction between pLenti4/TO/V5-DEST and pENTR/dT-lacZ no stop (Invitrogen Corporation, Carlsbad, Calif.). Clones of pLenti4/TO/V5-GW/lacZ were confirmed by restriction and sequence analyses. A map of pLenti4/TO/V5-GW/lacZ is shown in FIG. 70B.

293FT (Invitrogen Corporation, Carlsbad, Calif. catalog no. R70007) and GripTite 293 (Invitrogen Corporation, Carlsbad, Calif. catalog no. R79507) cells a were cultured in DMEM/10% FBS/L-glutamine/non-essential amino acids/penicillin/streptomycin containing 500 μg/ml G418. MJ90 primary human foreskin fibroblasts (Grand Island) and HT1080 human fibrosarcoma (ATCC #CCL-121) were cultured in DMEM/10% FBS/non-essential amino acids/penicillin/streptomycin. 10 μg/ml blasticidin was used to select for stable pLenti6/TR-transduced cells. MJ90 primary cells were growth arrested by contact inhibition. Briefly, 1×10⁵ cells were plated per well of a 6-well plate and media changes were performed every 3 days for 7 to 14 days, or until a quiescent monolayer was achieved.

For virus production, 5×10⁶ 293FT cells were plated per 100 mm plate. Twenty-four hours later, the culture medium was replaced with 5 ml OptiMem/10% FBS and cells were quadruple co-transfected, as follows. 12 μg DNA total, at a mass ratio of 1:1:1:1 pLenti6/TR or pLentiTO/V5-GW/lacZ :pLP1:LP2:pLP/VSVG (3 μg of each DNA) was mixed with 1.5 ml of OptiMem media. In a separate tube, 36 μl of Lipofectamine 2000 was also mixed with 1.5 ml of OptiMem media. After a 5-minute incubation period at room temperature, the two mixtures were combined and incubated at room temperature for an additional 20 minutes. At the completion of the incubation period, the transfection mixture was added to the cells dropwise and the culture plate was gently swirled to mix. The following day the transfection complex was replaced with complete media (DMEM, 10% FBS, 1% penicillin/streptomycin, L-glutamine and non-essential amino acids). Forty-eight to seventy-two hours post transfection, the virus-containing supernatants were harvested, centrifuged at 3000 rpm for 5 minutes to remove dead cells and placed in cryovials in 1 ml aliquots. Titers were performed on fresh supernatants (see below) and the remaining viral aliquots were stored at −70° C.

All applications of virus to cells were performed in the presence of 6 μg/ml polybrene (Sigma #H9268) and media changes were performed 12-24 hours post transduction. For titering virus, 6-well plates were seeded at 2×10⁵ cells per well with HT1080 cells the day before transduction. One well served as an untransduced control (mock) and the remaining five wells contained 1 ml each of ten-fold serial dilutions of viral supernatant ranging from 10⁻² to 10^{31 6}. The dilutions were mixed by gentle inversion prior to adding to cells. 6 μg/ml of polybrene was added to each well. The plate was gently swirled to mix. The following day, the media was replaced with complete media. Forty-eight hours post-transduction, the cells were placed under 10 μg/ml blasticidin or 100 μg/ml zeocin selection, as appropriate. In particular, Zeocin selection was done as follows: 24-hour post-transduction cells were trypsinized from 6-well plates and expanded into 100 mm plates. 24 hrs after expansion into 100 mm plates, 100 μg/ml Zeocin was added to the transduced cell culture medium for selection. After 7 to 10 days of blasticidin selection, or two-to-three weeks of zeocin selection, the resulting colonies were stained with crystal violet: A 1% crystal violet solution was prepared in 10% ethanol. Each well was washed with 2 ml PBS followed by 1 ml of crystal violet solution for 10 minutes at room temperature. Excess stain was removed by two 2 ml PBS washes and colonies visible to the naked eye were counted to determine the viral titer of the original supernatants.

Cell lysates for western blot and Tropix Assays were prepared as follows: Culture media were aspirated and cells were washed 1× with PBS and followed by incubation in Versene (Invitrogen Corporation, Carlsbad, Calif. catalog no. 15040066) for 2 minutes at room temperature. Detached cells were pelleted in Eppendorf Tubes and lysed in ice-cold 100 μl NP-40 lysis buffer (50 mM Tris, 150 mM NaCl, 1% NP-40, pH 8.0) containing protease inhibitors. Lysates were centrifuiged at 14000 rpm for 5 min to pellet cellular debris; the supernatant was collected and frozen at −70° C. until needed for assays. Protein concentrations were determined using BioRad Protein Assay protocol according to the manufacturer's (Biorad) recommendations.

Western blots were performed using 20 μg of normalized protein in a 4× loading dye. Samples were run on a Novex^{R} Tris-Glycine 4-20% Gel (Invitrogen Corporation, Carlsbad, Calif. catalog no. EC60252BOX), at 200 volts for 45 minutes. Protein was transferred to a nitrocellulose membrane and were detected with a WesternBreeze^{R} Chemiluminescent Kit (Invitrogen Corporation, Carlsbad, Calif. catalog no. WB7104) using polyclonal anti-TetR and monoclonal anti-V5 (Invitrogen Corporation, Carlsbad, Calif. catalog no. R96025) primary antibodies, as appropriate.

pLenti6/TR and pLenti4/TO/V5-GW/lacZ were transfected into 293FT cells, in the presence of Virapower Packaging Mix, to produce the respective viruses. pLenti6/TR and pLenti4/TO/V5-GW/lacZ produced viral titers of 6×10⁵ and 1×10⁵ cfu/ml respectively. Thus introduction of β-globin intron and TetR into pLenti6, and introduction of Tet Operators into pLenti4-DEST, do not compromise virus packaging and transduction efficiency.

Materials and methods of the invention may be used for a wide variety of purposes. For example, a nucleic acid molecule expressing a repressor protein (e.g., Lenti6/TR virus) may be used to generate repressor expressing cell lines. Such cell lines may be transduced with a nucleic acid molecule comprising promoter and repressor sequences operably linked to a sequence of interest (e.g., Lenti4/TO/5-GW/sequence of interest) and then expression of the sequence of interest may be regulated (e.g., using tetracycline). Another use of the materials and methods of the invention is to simultaneously cotransduce a nucleic acid molecule encoding a repressor and a nucleic acid molecule comprising promoter and repressor sequences operably linked to sequence of interest (e.g., Lenti6/TR and Lenti4/TO/V5-GW/sequence of interest) into primary non-dividing cells, then regulate expression of the sequence of interest (e.g., using tetracycline).

HT1080 cells were transduced with Lenti6/TR virus at MOI of 1, 10 or 32 and were selected in blasticidin medium until mock transduced cells had died-off. The blasticidin-resistant cells were next transduced with Lenti4/TO/V5-GW/lacZ virus at MOI of 5. Twenty-four hours after transducing with Lenti4/TO/V5-GW/lacZ, 1 μg/ml tetracycline was added to the culture medium. Cells were incubated in the inducer-supplemented medium for 48 hrs. Thereafter, cell lysates were prepared and analyzed for gene expression by (i) assaying for lacZ activity; (ii) performing western blot for lacZ-V5 fusion using anti-V5 antibody; (iii) western blot for TetR.

Increasing the amount of transduced TetR virus reduced lacZ expression in the absence of tetracycline. Tetracycline at 1 μg/ml induces lacZ expression to levels approaching full-strength CMV promoter activity. To determine fold induction, the ratios of β-galactosidase activities in the presence and absence of tetracycline (for a given MOI) were calculated. Induction of lacZ expression was 4-, 17- and 27-fold at TetR MOI of 1, 10 and 32, respectively, indicating that induction was dependent on the amount of TetR. Western blot analyses using anti-V5 antibody was consistent with the β-galactosidase enzymatic activity data. Expression of TetR protein was confirmed by western blot using polyclonal anti-TetR antibody.

These results confirm CMVTetO₂ and TetR in the lentiviral vectors to be functional and responsive to tetracycline. The relatively high level basal transcription from CMVTetO₂ at lower Lenti6/TR MOIs could result from the fact that not all blasticidin resistant cells generated at the low TetR MOIs actually express TetR. Those cells that do not express TetR would express lacZ from CMVTetO₂ promoter without inhibition and produce a high background. By contrast, at high Lenti/TR MOIs, close to 100% of blasticidin-resistant cells generated would express TetR, inhibit transcription from CMVTetO₂ promoter and produce lower background lacZ expression.

The data in HT1080 cells showed that lower basal transcription in a cell population is achieved at higher TetR levels. Therefore when testing induction in GripTite 293 cells, Lenti6/TR was transduced at MOI=10 and MOI=32 to generate blasticidin-resistant GripTite-10 and GripTite-32 populations, respectively. These populations were transduced with Lenti4/TO/V5-GW/lacZ virus at MOI=1 or MOI=5 and tested for lacZ induction. Tetracycline was used at 1 μg/ml or at 5 μg/ml to determine if inducer was limiting at higher TetR concentrations.

TetR effectively inhibited lacZ expression in GripTite-10 cells in the absence of inducer and this repression was relieved by tetracycline. 1 μg/ml tetracycline was nearly as effective as 5 μg/ml tetracycline in inducing gene expression. Fold induction was calculated as induced:uninduced ratios at a given Lenti4/TO/V5-GW/lacZ MOI and tetracycline concentration. LacZ expression was induced over 27-fold at Lenti4/TO/V5-GW/lacZ MOI=5 compared to just above 7-fold at Lenti4/TO/V5-GW/lacZ MOI=1. Western blot analyses using anti-VS antibody reflected β-gal enzymatic Tropix data. Expression of TetR protein was confirmed by western blot using polyclonal anti-TetR antibody.

The results in GripTite 293-10 cells were recapitulated in GripTite-32 cells. As in GripTite 293-10 cells, 1 μg/ml was nearly as effective as 5 μg/ml tetracycline in inducing gene expression in GripTite-32 cells. The fold lacZ induction was significantly higher in GripTite 293-32 cells however and ranged from 57 to 72 fold at Lenti4/TO/V5-GW/lacZ MOI=1 and Lenti4/TO/V5-GW/lacZ MOI=5, respectively.

The data indicate that 1 μg/ml tetracycline is not limiting in inducing lacZ expression. LacZ induction was higher at Lenti4/TO/V5-GW/lacZ MOI=5 than at MOI=1. Thus the amount of expression may be adjusted by altering the MOI of the virus containing a sequence of interest operably linked to a promoter and repressor sequence (e.g., higher MOI for higher expression level when de-repressed, lower MOI for lower expression level when de-repressed). The increased MOI has little effect on background uninduced levels when TetR is not limiting (e.g., MOI of 10 and 32).

In one particular embodiment, materials and methods of the invention may be used to regulate gene expression in non-dividing primary cells. MJ90 cells are contact-inhibited primary fibroblasts that undergo growth arrest at confluence and are refractory to both lipid transfection and transduction by Moloney retroviral vectors. MJ90 cells were transduced with 2×10⁶ cfu/well Lenti6/TR virus for 24 hrs followed by transduction with 2×10⁶ cfu/well of Lenti4/TO/V5-GW/lacZ virus (estimated MOI=7.5 each). Twenty-four hours after transducing with Lenti4/TO/V5-GW/lacZ, lacZ expression was induced with 1 μg/ml tetracycline for 48 hrs. Lysates from transduced cells were analyzed for protein induction. TetR repressed expression of lacZ over 90%, resulting in a 10-fold induction. It is worth noting that the preceding experiment was performed with equal MOI of Lenti6/TR and Lenti4/TO/V5-GW/lacZ. Higher Lenti6/TR MOI, or different Lenti6/TR: Lenti4/TO/V5-GW/lacZ ratios may be used to give higher inducibility. The demonstration that the present invention can regulate gene expression in quiescent primary cells is significant especially since the cells are hard to transfect and resist transduction by Moloney retroviral vectors.

Nucleic acid molecules of the invention comprising promoter and repressor sequences operably linked to a sequence of interest may be used in conjunction with any nucleic acid molecule expressing a repressor protein. For example, Lenti4/TO/V5-GW/lacZ virus was transduced into the Flp-In T-REx 293 product cell line (Invitrogen Corporation, Carlsbad, Calif. catalog no. R78007) at MOIs of 1 and 2.5. Gene expression was induced with 1 μg/ml tetracycline for 48 hrs. Tetracycline induced lacZ expression from Lenti4/TO/V5-GW/lacZ in Flp-In T-REx 293 cells. Increasing the amount of transduced Lenti4/TO/V5-GW/lacZ from MOI=1 to MOI=2.5 increased induction from 16-fold to 24-fold, respectively similar to the results in GripTite-10 and GripTite-32 populations.

### Example 18

In some embodiments, the present invention provides a method of covalently attaching an enzyme (e.g., a topoisomerase) to a nucleic acid molecule. In one aspect, a nucleic acid molecule for use in methods of this type may comprise a restriction enzyme recognition sequence (e.g., a TypeIls restriction enzyme recognition) and a topoisomerase recognition sequence. In some embodiments, a TypeIIs recognition sequence may be located adjacent to a topoisomerase recognition sequence. In this regard, adjacent means that the cleavage sites of the two enzymes may be within from about 1 to about 50, from about 1 to about 45, from about 1 to about 40, from about 1 to about 35, from about 1 to about 30, from about 1 to about 25, from about 1 to about 20, from about 1 to about 15, from about 1 to about 10, from about 1 to about 9, from about 1 to about 8, from about 1 to about 7, from about 1 to about 6, from about 1 to about 5, from about 1 to about 4, from about 1, to about 3, or from about 1 to about 2 base pairs from each other. Any TypeIIs enzyme may be used. In some embodiments, a suitable TypeIIs enzyme may leave a 3′-overhanging sequence. Suitable TypeIIs enzymes include Bael.

With reference to FIG. 73, a nucleic acid molecule of the invention may comprise two topoisomerase recognition sites and two TypeIIs recognition sites, for example with the two restriction enzyme sites between the two topisomerase sites. Optionally a nucleic acid molecule of this type may comprise nucleic acid sequence between the restriction enzyme sites. The nucleic acid sequence between the restriction enzyme sites may encode a polypeptide, for example, a selectable marker such as the ccdB gene.

The restriction enzyme sites may be located such that a 3′-overhang of a desired length is produced on the strand containing the topoisomerase cleavage site (after the 3′-T in FIG. 73). The location of the topoisomerase cleavage site may be varied with respect to 3′-most nucleotide of the strand containing the cleavage site. This may be useful in generating a 5′-overhang on the opposite strand after topoisomerase cleavage in order to generate a sequence that can invade a double-stranded insert (see FIG. 47).

After restriction enzyme cleavage, the cleaved vector may be contacted with an oligonucleotide that anneals to the 3′-overhanging sequence and/or may be contacted with a topoisomerase.

In some embodiments, methods of the invention may comprise digesting a nucleic acid molecule of the invention (e.g., 20 μg) with a TypeIIs restriction enzyme (e.g., 100 Units of BaeI, New England Biolabs, catalog no. R0613S), for example, in a final volume of 250 μl. Any other restriction enzyme known in the art may be also be used. The reaction may be carried out in a suitable buffer (e.g., 1×NEBuffer 2 with 100 μg/ml of BSA and 20 μM S-adenosylmethionine, New England Biolabs) under suitable conditions (e.g., at 37° C. for 6 hours). The digestion may be terminated, for example, with the addition of 250 μl of Phenol/Chloroform (Invitrogen Corporation, Carlsbad, Calif., Cat. #15593-031) and mixing. The organic and aqueous phases may be separated by centrifugation at 14,000×g at 4° C. for 5 minutes. The aqueous (top) layer may be transferred to a new tube and 25 μl of 3M sodium acetate (pH 5.2) may be added and mixed. This may be followed by 625 μl of 100% ethanol and incubation in ice for 5 minutes. Precipitated DNA may be was harvested by centrifugation at 14,000×g for 5 minutes at 4° C. The DNA pellet may be washed with 500 μl of 70% ethanol, harvested by centrifugation at 14,000×g for 5 minutes at 22° C. The pellet may be allowed to dry and then resuspended in 100 μl of TE. The DNA concentration may be determined by its optical density at 260 nm.

The digested vector may be contacted with an oligonucleotide that anneals to all or a portion of the 3′-overhang produced by the restriction enzyme and/or with a suitable topoisomerase enzyme (e.g., Vaccinia DNA Topoisomerase) in a suitable buffer (e.g., 1×NEBuffer #1, New England Biolabs), for example, in a final volume of 50 μl. The reaction may be incubated under suitable conditions (e.g., 25° C. for 15 minutes). Then reaction may be terminated with the addition of 5 μl of 10×Stop Buffer. The topoisomerase-linked vector may be purified by gel electrophoresis (see, Heyman, et al. Genome Research 9:383-392 (1999)).

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

TABLE 6
Nucleotide sequence of pAd/CMV/V5-DEST.

catcatcaataatataccttattttggattgaagccaatatgataatgagggggtggagtttgtgacgtggcgcggggcgtgggaacgg
ggcgggtgacgtagtagtgtggcggaagtgtgatgttgcaagtgtggcggaacacatgtaagcgacggatgtggcaaaagtgacgtt
tttggtgtgcgccggtgtacacaggaagtgacaattttcgcgcggttttaggcggatgttgtagtaaatttgggcgtaaccgagtaagatt
tggccattttcgcgggaaaactgaataagaggaagtgaaatctgaataattttgtgttactcatagcgcgtaatatttgtctagggccgcg
gggactttgaccgtttacgtggagactcgcccaggtgtttttctcaggtgttttccgcgttccgggtcaaagttggcgttttattattatagtc
agtcgaagcttggatccggtacctctagaattctcgagcggccgctagcgacatcggatctcccgatcccctatggtcgactctcagta
caatctgctctgatgccgcatagttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgcttcgcgatgtacgggccagat
atacgcgttgacattgattattgactagttattaatagtaatcaattacggggtcattagttcatagcccatatatggagttccgcgttacata
acttacggtaaatggcccgcctggctgaccgcccaacgacccccgcccattgacgtcaataatgacgtatgttcccatagtaacgcca
atagggactttccattgacgtcaatgggtggactatttacggtaaactgcccacttggcagtacatcaagtgtatcatatgccaagtacgc
cccctattgacgtcaatgacggtaaatggcccgcctggcattatgcccagtacatgaccttatgggactttcctacttggcagtacatcta
cgtattagtcatcgctattaccatggtgatgcggttttggcagtacatcaatgggcgtggatagcggtttgactcacggggatttccaagt
ctccaccccattgacgtcaatgggagtttgttttggcaccaaaatcaacgggactttccaaaatgtcgtaacaactccgccccattgacg
caaatgggcggtaggcgtgtacggtgggaggtctatataagcagagctctctggctaactagagaacccactgcttactggcttatcga
aattaatacgactcactatagggagacccaagctggctagttaagctatcaacaagtttgtacaaaaaagctgaacgagaaacgtaaaa
tgatataaatatcaatatattaaattagattttgcataaaaaacagactacataatactgtaaaacacaacatatccagtcactatgaatcaa
ctacttagatggtattagtgacctgtagtcgaccgacagccttccaaatgttcttcgggtgatgctgccaacttagtcgaccgacagcctt
ccaaatgttcttctcaaacggaatcgtcgtatccagcctactcgctattgtcctcaatgccgtattaaatcataaaaagaaataagaaaaag
aggtgcgagcctcttttttgtgtgacaaaataaaaacatctacctattcatatacgctagtgtcatagtcctgaaaatcatctgcatcaagaa
caatttcacaactcttatacttttctcttacaagtcgttcggcttcatctggattttcagcctctatacttactaaacgtgataaagtttctgtaatt
tctactgtatcgacctgcagactggctgtgtataagggagcctgacatttatattccccagaacatcaggttaatggcgtttttgatgtcattt
tcgcggtggctgagatcagccacttcttccccgataacggagaccggcacactggccatatcggtggtcatcatgcgccagctttcatc
cccgatatgcaccaccgggtaaagttcacgggagactttatctgacagcagacgtgcactggccagggggatcaccatccgtcgccc
gggcgtgtcaataatatcactctgtacatccacaaacagacgataacggctctctcttttataggtgtaaaccttaaactgcatttcaccag
tccctgttctcgtcagcaaaagagccgttcatttcaataaaccgggcgacctcagccatcccttcctgattttccgctttccagcgttcggc
acgcagacgacgggcttcattctgcatggttgtgcttaccagaccggagatattgacatcatatatgccttgagcaactgatagctgtcg
ctgtcaactgtcactgtaatacgctgcttcatagcacacctctttttgacatacttcgggtatacatatcagtatatattcttataccgcaaaaa
tcagcgcgcaaatacgcatactgttatctggcttttagtaagccggatccacgcgattacgccccgccctgccactcatcgcagtactgt
tgtaattcattaagcattctgccgacatggaagccatcacagacggcatgatgaacctgaatcgccagcggcatcagcaccttgtcgcc
ttgcgtataatatttgcccatggtgaaaacgggggcgaagaagttgtccatattggccacgtttaaatcaaaactggtgaaactcaccca
gggattggctgagacgaaaaacatattctcaataaaccctttagggaaataggccaggttttcaccgtaacacgccacatcttgcgaata
tatgtgtagaaactgccggaaatcgtcgtggtattcactccagagcgatgaaaacgtttcagtttgctcatggaaaacggtgtaacaagg
gtgaacactatcccatatcaccagctcaccgtctttcattgccatacggaattccggatgagcattcatcaggcgggcaagaatgtgaat
aaaggccggataaaacttgtgcttatttttctttacggtctttaaaaaggccgtaatatccagctgaacggtctggttataggtacattgagc
aactgactgaaatgcctcaaaatgttctttacgatgccattgggatatatcaacggtggtatatccagtgatttttttctccattttagcttcctt
agctcctgaaaatctcgataactcaaaaaatacgcccggtagtgatcttatttcattatggtgaaagttggaacctcttacgtgccgatcaa
cgtctcattttcgccaaaagttggcccagggcttcccggtatcaacagggacaccaggatttatttattctgcgaagtgatcttccgtcac
aggtatttattcggcgcaaagtgcgtcgggtgatgctgccaacttagtcgactacaggtcactaataccatctaagtagttgattcatagt
tttcttgtacaaagtggttgatctagagggcccgcggttcgaaggtaagcctatccctaaccctctcctcggtctcgattctacgcgtacc
ggttagtaatgagtttaaacgggggaggctaactgaaacacggaaggagacaataccggaaggaacccgcgctatgacggcaataa
aaagacagaataaaacgcacgggtgttgggtcgtttgttcataaacgcggggttcggtcccagggctggcactctgtcgataccccac
cgagaccccattggggccaata
cgcccgcgtttcttccttttccccaccccaccccccaagttcgggtgaaggcccagggctcgcagccaacgtcggggcggcaggcc
ctgccatagcagatccgattcgacagatcactgaaatgtgtgggcgtggcttaagggtgggaaagaatatataaggtgggggtcttatg
tagttttgtatctgttttgcagcagccgccgccgccatgagcaccaactcgtttgatggaagcattgtgagctcatatttgacaacgcgcat
gcccccatgggccggggtgcgtcagaatgtgatgggctccagcattgatggtcgccccgtcctgcccgcaaactctactaccttgacc
tacgagaccgtgtctggaacgccgttggagactgcagcctccgccgccgcttcagccgctgcagccaccgcccgcgggattgtgac
tgactttgctttcctgagcccgcttgcaagcagtgcagcttcccgttcatccgcccgcgatgacaagttgacggctcttttggcacaattg
gattctttgacccgggaacttaatgtcgtttctcagcagctgttggatctgcgccagcaggtttctgccctgaaggcttcctcccctcccaa
tgcggtttaaaacataaataaaaaaccagactctgtttggatttggatcaagcaagtgtcttgctgtctttatttaggggttttgcgcgcgcg
gtaggcccgggaccagcggtctcggtcgttgagggtcctgtgtattttttccaggacgtggtaaaggtgactctggatgttcagatacat
gggcataagcccgtctctggggtggaggtagcaccactgcagagcttcatgctgcggggtggtgttgtagatgatccagtcgtagcag
gagcgctgggcgtggtgcctaaaaatgtctttcagtagcaagctgattgccaggggcaggcccttggtgtaagtgtttacaaagcggtt
aagctgggatgggtgcatacgtggggatatgagatgcatcttggactgtatttttaggttggctatgttcccagccatatccctccgggga
ttcatgttgtgcagaaccaccagcacagtgtatccggtgcacttgggaaatttgtcatgtagcttagaaggaaatgcgtggaagaacttg
gagacgcccttgtgacctccaagattttccatgcattcgtccataatgatggcaatgggcccacgggcggcggcctgggcgaagatat
ttctgggatcactaacgtcatagttgtgttccaggatgagatcgtcataggccatttttacaaagcgcgggcggagggtgccagactgc
ggtataatggttccatccggcccaggggcgtagttaccctcacagatttgcatttcccacgctttgagttcagatggggggatcatgtcta
cctgcggggcgatgaagaaaacggtttccggggtaggggagatcagctgggaagaaagcaggttcctgagcagctgcgacttacc
gcagccggtgggcccgtaaatcacacctattaccgggtgcaactggtagttaagagagctgcagctgccgtcatccctgagcagggg
ggccacttcgttaagcatgtccctgactcgcatgttttccctgaccaaatccgccagaaggcgctcgccgcccagcgatagcagttctt
gcaaggaagcaaagtttttcaacggtttgagaccgtccgccgtaggcatgcttttgagcgtttgaccaagcagttccaggcggtcccac
agctcggtcacctgctctacggcatctcgatccagcatatctcctcgtttcgcgggttggggcggctttcgctgtacggcagtagtcggt
gctcgtccagacgggccagggtcatgtctttccacgggcgcagggtcctcgtcagcgtagtctgggtcacggtgaaggggtgcgctc
cgggctgcgcgctggccagggtgcgcttgaggctggtcctgctggtgctgaagcgctgccggtcttcgccctgcgcgtcggccagg
tagcatttgaccatggtgtcatagtccagcccctccgcggcgtggcccttggcgcgcagcttgcccttggaggaggcgccgcacgag
gggcagtgcagacttttgagggcgtagagcttgggcgcgagaaataccgattccggggagtaggcatccgcgccgcaggccccgc
agacggtctcgcattccacgagccaggtgagctctggccgttcggggtcaaaaaccaggtttcccccatgctttttgatgcgtttcttacc
tctggtttccatgagccggtgtccacgctcggtgacgaaaaggctgtccgtgtccccgtatacagacttgagaggcctgtcctcgagcg
gtgttccgcggtcctcctcgtatagaaactcggaccactctgagacaaaggctcgcgtccaggccagcacgaaggaggctaagtgg
gaggggtagcggtcgttgtccactagggggtccactcgctccagggtgtgaagacacatgtcgccctcttcggcatcaaggaaggtg
attggtttgtaggtgtaggccacgtgaccgggtgttcctgaaggggggctataaaagggggtgggggcgcgttcgtcctcactctcttc
cgcatcgctgtctgcgagggccagctgttggggtgagtactccctctgaaaagcgggcatgacttctgcgctaagattgtcagtttcca
aaaacgaggaggatttgatattcacctggcccgcggtgatgcctttgagggtggccgcatccatctggtcagaaaagacaatctttttgt
tgtcaagcttggtggcaaacgacccgtagagggcgttggacagcaacttggcgatggagcgcagggtttggtttttgtcgcgatcggc
gcgctccttggccgcgatgtttagctgcacgtattcgcgcgcaacgcaccgccattcgggaaagacggtggtgcgctcgtcgggcac
caggtgcacgcgccaaccgcggttgtgcagggtgacaaggtcaacgctggtggctacctctccgcgtaggcgctcgttggtccagc
agaggcggccgcccttgcgcgagcagaatggcggtagggggtctagctgcgtctcgtccggggggtctgcgtccacggtaaagac
cccgggcagcaggcgcgcgtcgaagtagtctatcttgcatccttgcaagtctagcgcctgctgccatgcgcgggcggcaagcgcgc
gctcgtatgggttgagtgggggaccccatggcatggggtgggtgagcgcggaggcgtacatgccgcaaatgtcgtaaacgtagagg
ggctctctgagtattccaagatatgtagggtagcatcttccaccgcggatgctggcgcgcacgtaatcgtatagttcgtgcgagggagc
gaggaggtcgggaccgaggttgctacgggcgggctgctctgctcggaagactatctgcctgaagatggcatgtgagttggatgatat
ggttggacgctggaagacgttgaagctggcgtctgtgagacctaccgcgtcacgcacgaaggaggcgtaggagtcgcgcagcttgt
tgaccagctcggcggtgacctgcacgtctagggcgcagtagtccagggtttccttgatgatgtcatacttatcctgtcccttttttttccaca
gctcgcggttgaggacaaactcttcgcggtctttccagtactcttggatcggaaacccgtcggcctccgaacggtaagagcctagcatg
tagaactggttgacggcctggtaggcgcagcatcccttttctacgggtagcgcgtatgcctgcgcggccttccggagcgaggtgtggg
tgagcgcaaagg
tgtccctgaccatgactttgaggtactggtatttgaagtcagtgtcgtcgcatccgccctgctcccagagcaaaaagtccgtgcgcttttt
ggaacgcggatttggcagggcgaaggtgacatcgttgaagagtatctttcccgcgcgaggcataaagttgcgtgtgatgcggaaggg
tcccggcacctcggaacggttgttaattacctgggcggcgagcacgatctcgtcaaagccgttgatgttgtggcccacaatgtaaagtt
ccaagaagcgcgggatgcccttgatggaaggcaattttttaagttcctcgtaggtgagctcttcaggggagctgagcccgtgctctgaa
agggcccagtctgcaagatgagggttggaagcgacgaatgagctccacaggtcacgggccattagcatttgcaggtggtcgcgaaa
ggtcctaaactggcgacctatggccattttttctggggtgatgcagtagaaggtaagcgggtcttgttcccagcggtcccatccaaggtt
cgcggctaggtctcgcgcggcagtcactagaggctcatctccgccgaacttcatgaccagcatgaagggcacgagctgcttcccaaa
ggcccccatccaagtataggtctctacatcgtaggtgacaaagagacgctcggtgcgaggatgcgagccgatcgggaagaactgga
tctcccgccaccaattggaggagtggctattgatgtggtgaaagtagaagtccctgcgacgggccgaacactcgtgctggcttttgtaa
aaacgtgcgcagtactggcagcggtgcacgggctgtacatcctgcacgaggttgacctgacgaccgcgcacaaggaagcagagtg
ggaatttgagcccctcgcctggcgggtttggctggtggtcttctacttcggctgcttgtccttgaccgtctggctgctcgaggggagttac
ggtggatcggaccaccacgccgcgcgagcccaaagtccagatgtccgcgcgcggcggtcggagcttgatgacaacatcgcgcag
atgggagctgtccatggtctggagctcccgcggcgtcaggtcaggcgggagctcctgcaggtttacctcgcatagacgggtcaggg
cgcgggctagatccaggtgatacctaatttccaggggctggttggtggcggcgtcgatggcttgcaagaggccgcatccccgcggc
gcgactacggtaccgcgcggcgggcggtgggccgcgggggtgtccttggatgatgcatctaaaagcggtgacgcgggcgagccc
ccggaggtagggggggctccggacccgccgggagagggggcaggggcacgtcggcgccgcgcgcgggcaggagctggtgct
gcgcgcgtaggttgctggcgaacgcgacgacgcggcggttgatctcctgaatctggcgcctctgcgtgaagacgacgggcccggtg
agcttgagcctgaaagagagttcgacagaatcaatttcggtgtcgttgacggcggcctggcgcaaaatctcctgcacgtctcctgagtt
gtcttgataggcgatctcggccatgaactgctcgatctcttcctcctggagatctccgcgtccggctcgctccacggtggcggcgaggt
cgttggaaatgcgggccatgagctgcgagaaggcgttgaggcctccctcgttccagacgcggctgtagaccacgcccccttcggcat
cgcgggcgcgcatgaccacctgcgcgagattgagctccacgtgccgggcgaagacggcgtagtttcgcaggcgctgaaagaggta
gttgagggtggtggcggtgtgttctgccacgaagaagtacataacccagcgtcgcaacgtggattcgttgatatcccccaaggcctca
aggcgctccatggcctcgtagaagtccacggcgaagttgaaaaactgggagttgcgcgccgacacggttaactcctcctccagaaga
cggatgagctcggcgacagtgtcgcgcacctcgcgctcaaaggctacaggggcctcttcttcttcttcaatctcctcttccataagggcc
tccccttcttcttcttctggcggcggtgggggaggggggacacggcggcgacgacggcgcaccgggaggcggtcgacaaagcgct
cgatcatctccccgcggcgacggcgcatggtctcggtgacggcgcggccgttctcgcgggggcgcagttggaagacgccgcccgt
catgtcccggttatgggttggcggggggctgccatgcggcagggatacggcgctaacgatgcatctcaacaattgttgtgtaggtactc
cgccgccgagggacctgagcgagtccgcatcgaccggatcggaaaacctctcgagaaaggcgtctaaccagtcacagtcgcaagg
taggctgagcaccgtggcgggcggcagcgggcggcggtcggggttgtttctggcggaggtgctgctgatgatgtaattaaagtagg
cggtcttgagacggcggatggtcgacagaagcaccatgtccttgggtccggcctgctgaatgcgcaggcggtcggccatgccccag
gcttcgttttgacatcggcgcaggtctttgtagtagtcttgcatgagcctttctaccggcacttcttcttctccttcctcttgtcctgcatctctt
gcatctatcgctgcggcggcggcggagtttggccgtaggtggcgccctcttcctcccatgcgtgtgaccccgaagcccctcatcggct
gaagcagggctaggtcggcgacaacgcgctcggctaatatggcctgctgcacctgcgtgagggtagactggaagtcatccatgtcca
caaagcggtggtatgcgcccgtgttgatggtgtaagtgcagttggccataacggaccagttaacggtctggtgacccggctgcgaga
gctcggtgtacctgagacgcgagtaagccctcgagtcaaatacgtagtcgttgcaagtccgcaccaggtactggtatcccaccaaaaa
gtgcggcggcggctggcggtagaggggccagcgtagggtggccggggctccgggggcgagatcttccaacataaggcgatgata
tccgtagatgtacctggacatccaggtgatgccggcggcggtggtggaggcgcgcggaaagtcgcggacgcggttccagatgttgc
gcagcggcaaaaagtgctccatggtcgggacgctctggccggtcaggcgcgcgcaatcgttgacgctctagaccgtgcaaaagga
gagcctgtaagcgggcactcttccgtggtctggtggataaattcgcaagggtatcatggcggacgaccggggttcgagccccgtatcc
ggccgtccgccgtgatccatgcggttaccgcccgcgtgtcgaacccaggtgtgcgacgtcagacaacgggggagtgctccttttggc
ttccttccaggcgcggcggctgctgcgctagcttttttggccactggccgcgcgcagcgtaagcggttaggctggaaagcgaaagcat
taagtggctcgctccctgtagccggagggttattttccaagggttgagtcgcgggacccccggttcgagtctcggaccggccggactg
cggcgaacgggggtttgcctccccgtcatgcaagaccccgcttgcaaattcctccggaaacagggacgagccccttttttgcttttccc
agatgcatccggtgctgcggcagatgcgcccccctcctcagcagcggcaagagcaagagcagcggcagacatgcagggcaccct
cccctcctcctacc
gcgtcaggaggggcgacatccgcggttgacgcggcagcagatggtgattacgaacccccgcggcgccgggcccggcactacctg
gacttggaggagggcgagggcctggcgcggctaggagcgccctctcctgagcggtacccaagggtgcagctgaagcgtgatacg
cgtgaggcgtacgtgccgcggcagaacctgtttcgcgaccgcgagggagaggagcccgaggagatgcgggatcgaaagttccac
gcagggcgcgagctgcggcatggcctgaatcgcgagcggttgctgcgcgaggaggactttgagcccgacgcgcgaaccgggatt
agtcccgcgcgcgcacacgtggcggccgccgacctggtaaccgcatacgagcagacggtgaaccaggagattaactttcaaaaaa
gctttaacaaccacgtgcgtacgcttgtggcgcgcgaggaggtggctataggactgatgcatctgtgggactttgtaagcgcgctgga
gcaaaacccaaatagcaagccgctcatggcgcagctgttccttatagtgcagcacagcagggacaacgaggcattcagggatgcgc
tgctaaacatagtagagcccgagggccgctggctgctcgatttgataaacatcctgcagagcatagtggtgcaggagcgcagcttga
gcctggctgacaaggtggccgccatcaactattccatgcttagcctgggcaagttttacgcccgcaagatataccataccccttacgttc
ccatagacaaggaggtaaagatcgaggggttctacatgcgcatggcgctgaaggtgcttaccttgagcgacgacctgggcgtttatcg
caacgagcgcatccacaaggccgtgagcgtgagccggcggcgcgagctcagcgaccgcgagctgatgcacagcctgcaaaggg
ccctggctggcacgggcagcggcgatagagaggccgagtcctactttgacgcgggcgctgacctgcgctgggccccaagccgac
gcgccctggaggcagctggggccggacctgggctggcggtggcacccgcgcgcgctggcaacgtcggcggcgtggaggaatat
gacgaggacgatgagtacgagccagaggacggcgagtactaagcggtgatgtttctgatcagatgatgcaagacgcaacggaccc
ggcggtgcgggcggcgctgcagagccagccgtccggccttaactccacggacgactggcgccaggtcatggaccgcatcatgtcg
ctgactgcgcgcaatcctgacgcgttccggcagcagccgcaggccaaccggctctccgcaattctggaagcggtggtcccggcgcg
cgcaaaccccacgcacgagaaggtgctggcgatcgtaaacgcgctggccgaaaacagggccatccggcccgacgaggccggcc
tggtctacgacgcgctgcttcagcgcgtggctcgttacaacagcggcaacgtgcagaccaacctggaccggctggtgggggatgtg
cgcgaggccgtggcgcagcgtgagcgcgcgcagcagcagggcaacctgggctccatggttgcactaaacgccttcctgagtacac
agcccgccaacgtgccgcggggacaggaggactacaccaactttgtgagcgcactgcggctaatggtgactgagacaccgcaaag
tgaggtgtaccagtctgggccagactattttttccagaccagtagacaaggcctgcagaccgtaaacctgagccaggctttcaaaaact
tgcaggggctgtggggggtgcgggctcccacaggcgaccgcgcgaccgtgtctagcttgctgacgcccaactcgcgcctgttgctg
ctgctaatagcgcccttcacggacagtggcagcgtgtcccgggacacatacctaggtcacttgctgacactgtaccgcgaggccatag
gtcaggcgcatgtggacgagcatactttccaggagattacaagtgtcagccgcgcgctggggcaggaggacacgggcagcctgga
ggcaaccctaaactacctgctgaccaaccggcggcagaagatcccctcgttgcacagtttaaacagcgaggaggagcgcattttgcg
ctacgtgcagcagagcgtgagccttaacctgatgcgcgacggggtaacgcccagcgtggcgctggacatgaccgcgcgcaacatg
gaaccgggcatgtatgcctcaaaccggccgtttatcaaccgcctaatggactacttgcatcgcgcggccgccgtgaaccccgagtattt
caccaatgccatcttgaacccgcactggctaccgccccctggtttctacaccgggggattcgaggtgcccgagggtaacgatggattc
ctctgggacgacatagacgacagcgtgttttccccgcaaccgcagaccctgctagagttgcaacagcgcgagcaggcagaggcgg
cgctgcgaaaggaaagcttccgcaggccaagcagcttgtccgatctaggcgctgcggccccgcggtcagatgctagtagcccatttc
caagcttgatagggtctcttaccagcactcgcaccacccgcccgcgcctgctgggcgaggaggagtacctaaacaactcgctgctgc
agccgcagcgcgaaaaaaacctgcctccggcatttcccaacaacgggatagagagcctagtggacaagatgagtagatggaagac
gtacgcgcaggagcacagggacgtgccaggcccgcgcccgcccacccgt&gtcaaaggcacgaccgtcagcggggtctggtgtg
ggaggacgatgactcggcagacgacagcagcgtcctggatttgggagggagtggcaacccgtttgcgcaccttcgccccaggctg
gggagaatgttttaaaaaaaaaaaagcatgatgcaaaataaaaaactcaccaaggccatggcaccgagcgttggttttcttgtattcccc
ttagtatgcggcgcgcggcgatgtatgaggaaggtcctcctccctcctacgagagtgtggtgagcgcggcgccagtggcggcggcg
ctgggttctcccttcgatgctcccctggacccgccgtttgtgcctccgcggtacctgcggcctaccggggggagaaacagcatccgtt
actctgagttggcacccctattcgacaccacccgtgtgtacctggtggacaacaagtcaacggatgtggcatccctgaactaccagaa
cgaccacagcaactttctgaccacggtcattcaaaacaatgactacagcccgggggaggcaagcacacagaccatcaatcttgacga
ccggtcgcactggggcggcgacctgaaaaccatcctgcataccaacatgccaaatgtgaacgagttcatgtttaccaataagtttaagg
cgcgggtgatggtgtcgcgcttgcctactaaggacaatcaggtggagctgaaatacgagtgggtggagttcacgctgcccgagggc
aactactccgagaccatgaccatagaccttatgaacaacgcgatcgtggagcactacttgaaagtgggcagacagaacggggttctg
gaaagcgacatcggggtaaagtttgacacccgcaacttcagactggggtttgaccccgtcactggtcttgtcatgcctggggtatatac
aaacgaagccttccatccagacatcattttgctgccaggatgcggggtggacttcacccacagccgcctgagcaacttgttgggcatcc
gcaagcggcaaccct
tccaggagggctttaggatcacctacgatgatctggagggtggtaacattcccgcactgttggatgtggacgcctaccaggcgagctt
gaaagatgacaccgaacagggcgggggtggcgcaggcggcagcaacagcagtggcagcggcgcggaagagaactccaacgc
ggcagccgcggcaatgcagccggtggaggacatgaacgatcatgccattcgcggcgacacctttgccacacgggctgaggagaag
cgcgctgaggccgaagcagcggccgaagctgccgcccccgctgcgcaacccgaggtcgagaagcctcagaagaaaccggtgat
caaacccctgacagaggacagcaagaaacgcagttacaacctaataagcaatgacagcaccttcacccagtaccgcagctggtacct
tgcatacaactacggcgaccctcagaccggaatccgctcatggaccctgctttgcactcctgacgtaacctgcggctcggagcaggtc
tactggtcgttgccagacatgatgcaagaccccgtgaccttccgctccacgcgccagatcagcaactttccggtggtgggcgccgag
ctgttgcccgtgcactccaagagcttctacaacgaccaggccgtctactcccaactcatccgccagtttacctctctgacccacgtgttca
atcgctttcccgagaaccagattttggcgcgcccgccagcccccaccatcaccaccgtcagtgaaaacgttcctgctctcacagatcac
gggacgctaccgctgcgcaacagcatcggaggagtccagcgagtgaccattactgacgccagacgccgcacctgcccctacgttta
caaggccctgggcatagtctcgccgcgcgtcctatcgagccgcactttttgagcaagcatgtccatccttatatcgcccagcaataaca
caggctggggcctgcgcttcccaagcaagatgtttggcggggccaagaagcgctccgaccaacacccagtgcgcgtgcgcgggca
ctaccgcgcgccctggggcgcgcacaaacgcggccgcactgggcgcaccaccgtcgatgacgccatcgacgcggtggtggagg
aggcgcgcaactacacgcccacgccgccaccagtgtccacagtggacgcggccattcagaccgtggtgcgcggagcccggcgct
atgctaaaatgaagagacggcggaggcgcgtagcacgtcgccaccgccgccgacccggcactgccgcccaacgcgcggcggcg
gccctgcttaaccgcgcacgtcgcaccggccgacgggcggccatgcgggccgctcgaaggctggccgcgggtattgtcactgtgc
cccccaggtccaggcgacgagcggccgccgcagcagccgcggccattagtgctatgactcagggtcgcaggggcaacgtgtattg
ggtgcgcgactcggttagcggcctgcgcgtgcccgtgcgcacccgccccccgcgcaactagattgcaagaaaaaactacttagact
cgtactgttgtatgtatccagcggcggcggcgcgcaacgaagctatgtccaagcgcaaaatcaaagaagagatgctccaggtcatcg
cgccggagatctatggccccccgaagaaggaagagcaggattacaagccccgaaagctaaagcgggtcaaaaagaaaaagaaag
atgatgatgatgaacttgacgacgaggtggaactgctgcacgctaccgcgcccaggcgacgggtacagtggaaaggtcgacgcgta
aaacgtgttttgcgacccggcaccaccgtagtctttacgcccggtgagcgctccacccgcacctacaagcgcgtgtatgatgaggtgt
acggcgacgaggacctgcttgagcaggccaacgagcgcctcggggagtttgcctacggaaagcggcataaggacatgctggcgtt
gccgctggacgagggcaacccaacacctagcctaaagcccgtaacactgcagcaggtgctgcccgcgcttgcaccgtccgaagaa
aagcgcggcctaaagcgcgagtctggtgacttggcacccaccgtgcagctgatggtacccaagcgccagcgactggaagatgtctt
ggaaaaaatgaccgtggaacctgggctggagcccgaggtccgcgtgcggccaatcaagcaggtggcgccgggactgggcgtgca
gaccgtggacgttcagatacccactaccagtagcaccagtattgccaccgccacagagggcatggagacacaaacgtccccggttg
cctcagcggtggcggatgccgcggtgcaggcggtcgctgcggccgcgtccaagacctctacggaggtgcaaacggacccgtggat
gtttcgcgtttcagccccccggcgcccgcgcggttcgaggaagtacggcgccgccagcgcgctactgcccgaatatgccctacatcc
ttccattgcgcctacccccggctatcgtggctacacctaccgccccagaagacgagcaactacccgacgccgaaccaccactggaac
ccgccgccgccgtcgccgtcgccagcccgtgctggccccgatttccgtgcgcagggtggctcgcgaaggaggcaggaccctggtg
ctgccaacagcgcgctaccaccccagcatcgtttaaaagccggtctttgtggttcttgcagatatggccctcacctgccgcctccgtttc
ccggtgccgggattccgaggaagaatgcaccgtaggaggggcatggccggccacggcctgacgggcggcatgcgtcgtgcgcac
caccggcggcggcgcgcgtcgcaccgtcgcatgcgcggcggtatcctgcccctccttattccactgatcgccgcggcgattggcgc
cgtgcccggaattgcatccgtggccttgcaggcgcagagacactgattaaaaacaagttgcatgtggaaaaatcaaaataaaaagtct
ggactctcacgctcgcttggtcctgtaactattttgtagaatggaagacatcaactttgcgtctctggccccgcgacacggctcgcgccc
gttcatgggaaactggcaagatatcggcaccagcaatatgagcggtggcgccttcagctggggctcgctgtggagcggcattaaaaa
tttcggttccaccgttaagaactatggcagcaaggcctggaacagcagcacaggccagatgctgagggataagttgaaagagcaaaa
tttccaacaaaaggtggtagatggcctggcctctggcattagcggggtggtggacctggccaaccaggcagtgcaaaataagattaac
agtaagcttgatccccgccctcccgtagaggagcctccaccggccgtggagacagtgtctccagaggggcgtggcgaaaagcgtcc
gcgccccgacagggaagaaactctggtgacgcaaatagacgagcctccctcgtacgaggaggcactaaagcaaggcctgcccacc
acccgtcccatcgcgcccatggctaccggagtgctgggccagcacacacccgtaacgctggacctgcctccccccgccgacaccc
agcagaaacctgtgctgccaggcccgaccgccgttgttgtaacccgtcctagccgcgcgtccctgcgccgcgccgccagcggtccg
cgatcgttgcggcccgtagccagtggcaactggcaaagcacactgaacagcatcgtgggtctgggggtgcaatccctgaagcgccg
acgatgcttct
gaatagctaacgtgtcgtatgtgtgtcatgtatgcgtccatgtcgccgccagaggagctgctgagccgccgcgcgcccgctttccaag
atggctaccccttcgatgatgccgcagtggtcttacatgcacatctcgggccaggacgcctcggagtacctgagccccgggctggtgc
agtttgcccgcgccaccgagacgtacttcagcctgaataacaagtttagaaaccccacggtggcgcctacgcacgacgtgaccacag
accggtcccagcgtttgacgctgcggttcatccctgtggaccgtgaggatactgcgtactcgtacaaggcgcggttcaccctagctgtg
ggtgataaccgtgtgctggacatggcttccacgtactttgacatccgcggcgtgctggacaggggccctacttttaagccctactctggc
actgcctacaacgccctggctcccaagggtgccccaaatccttgcgaatgggatgaagctgctactgctcttgaaataaacctagaag
aagaggacgatgacaacgaagacgaagtagacgagcaagctgagcagcaaaaaactcacgtatttgggcaggcgccttattctggt
ataaatattacaaaggagggtattcaaataggtgtcgaaggtcaaacacctaaatatgccgataaaacatttcaacctgaacctcaaata
ggagaatctcagtggtacgaaactgaaattaatcatgcagctgggagagtccttaaaaagactaccccaatgaaaccatgttacggttc
atatgcaaaacccacaaatgaaaatggagggcaaggcattcttgtaaagcaacaaaatggaaagctagaaagtcaagtggaaatgca
atttttctcaactactgaggcgaccgcaggcaatggtgataacttgactcctaaagtggtattgtacagtgaagatgtagatatagaaacc
ccagacactcatatttcttacatgcccactattaaggaaggtaactcacgagaactaatgggccaacaatctatgcccaacaggcctaat
tacattgcttttagggacaattttattggtctaatgtattacaacagcacgggtaatatgggtgttctggcgggccaagcatcgcagttgaa
tgctgttgtagatttgcaagacagaaacacagagctttcataccagcttttgcttgattccattggtgatagaaccaggtacttttctatgtgg
aatcaggctgttgacagctatgatccagatgttagaattattgaaaatcatggaactgaagatgaacttccaaattactgctttccactggg
aggtgtgattaatacagagactcttaccaaggtaaaacctaaaacaggtcaggaaaatggatgggaaaaagatgctacagaattttcag
ataaaaatgaaataagagttggaaataattttgccatggaaatcaatctaaatgccaacctgtggagaaatttcctgtactccaacatagc
gctgtatttgcccgacaagctaaagtacagtccttccaacgtaaaaatttctgataacccaaacacctacgactacatgaacaagcgagt
ggtggctcccgggttagtggactgctacattaaccttggagcacgctggtcccttgactatatggacaacgtcaacccatttaaccacca
ccgcaatgctggcctgcgctaccgctcaatgttgctgggcaatggtcgctatgtgcccttccacatccaggtgcctcagaagttctttgc
cattaaaaacctccttctcctgccgggctcatacacctacgagtggaacttcaggaaggatgttaacatggttctgcagagctccctagg
aaatgacctaagggttgacggagccagcattaagtttgatagcatttgcctttacgccaccttcttccccatggcccacaacaccgcctc
cacgcttgaggccatgcttagaaacgacaccaacgaccagtcctttaacgactatctctccgccgccaacatgctctaccctatacccg
ccaacgctaccaacgtgcccatatccatcccctcccgcaactgggcggctttccgcggctgggccttcacgcgccttaagactaagga
aaccccatcactgggctcgggctacgacccttattacacctactctggctctataccctacctagatggaaccttttacctcaaccacacc
tttaagaaggtggccattacctttgactcttctgtcagctggcctggcaatgaccgcctgcttacccccaacgagtttgaaattaagcgct
cagttgacggggagggttacaacgttgcccagtgtaacatgaccaaagactggttcctggtacaaatgctagctaactacaacattggc
taccagggcttctatatcccagagagctacaaggaccgcatgtactccttctttagaaacttccagcccatgagccgtcaggtggtggat
gatactaaatacaaggactaccaacaggtgggcatcctacaccaacacaacaactctggatttgttggctaccttgcccccaccatgcg
cgaaggacaggcctaccctgctaacttcccctatccgcttataggcaagaccgcagttgacagcattacccagaaaaagtttctttgcg
atcgcaccctttggcgcatcccattctccagtaactttatgtccatgggcgcactcacagacctgggccaaaaccttctctacgccaactc
cgcccacgcgctagacatgacttttgaggtggatcccatggacgagcccacccttctttatgttttgtttgaagtctttgacgtggtccgtg
tgcaccggccgcaccgcggcgtcatcgaaaccgtgtacctgcgcacgcccttctcggccggcaacgccacaacataaagaagcaa
gcaacatcaacaacagctgccgccatgggctccagtgagcaggaactgaaagccattgtcaaagatcttggttgtgggccatattttttg
ggcacctatgacaagcgctttccaggctttgtttctccacacaagctcgcctgcgccatagtcaatacggccggtcgcgagactgggg
gcgtacactggatggcctttgcctggaacccgcactcaaaaacatgctacctctttgagccctttggcttttctgaccagcgactcaagc
aggtttaccagtttgagtacgagtcactcctgcgccgtagcgccattgcttcttcccccgaccgctgtataacgctggaaaagtccaccc
aaagcgtacaggggcccaactcggccgcctgtggactattctgctgcatgtttctccacgcctttgccaactggccccaaactcccatg
gatcacaaccccaccatgaaccttattaccggggtacccaactccatgctcaacagtccccaggtacagcccaccctgcgtcgcaacc
aggaacagctctacagcttcctggagcgccactcgccctacttccgcagccacagtgcgcagattaggagcgccacttctttttgtcact
tgaaaaacatgtaaaaataatgtactagagacactttcaataaaggcaaatgcttttatttgtacactctcgggtgattatttacccccaccc
ttgccgtctgcgccgtttaaaaatcaaaggggttctgccgcgcatcgctatgcgccactggcagggacacgttgcgatactggtgttta
gtgctccacttaaactcaggcacaaccatccgcggcagctcggtgaagttttcactccacaggctgcgcaccatcaccaacgcgtttag
caggtcgggcgccgatatcttgaagtcgcagttggggcctccgccctgcgcgcgcgagttgcgatacacagggttgcagcactgga
acactatcagcgcc
gggtggtgcacgctggccagcacgctcttgtcggagatcagatccgcgtccaggtcctccgcgttgctcagggcgaacggagtcaa
ctttggtagctgccttcccaaaaagggcgcgtgcccaggctttgagttgcactcgcaccgtagtggcatcaaaaggtgaccgtgcccg
gtctgggcgttaggatacagcgcctgcataaaagccttgatctgcttaaaagccacctgagcctttgcgccttcagagaagaacatgcc
gcaagacttgccggaaaactgattggccggacaggccgcgtcgtgcacgcagcaccttgcgtcggtgttggagatctgcaccacattt
cggccccaccggttcttcacgatcttggccttgctagactgctccttcagcgcgcgctgcccgttttcgctcgtcacatccatttcaatcac
gtgctccttatttatcataatgcttccgtgtagacacttaagctcgccttcgatctcagcgcagcggtgcagccacaacgcgcagcccgt
gggctcgtgatgcttgtaggtcacctctgcaaacgactgcaggtacgcctgcaggaatcgccccatcatcgtcacaaaggtcttgttgc
tggtgaaggtcagctgcaacccgcggtgctcctcgttcagccaggtcttgcatacggccgccagagcttccacttggtcaggcagtag
tttgaagttcgcctttagatcgttatccacgtggtacttgtccatcagcgcgcgcgcagcctccatgcccttctcccacgcagacacgatc
ggcacactcagcgggttcatcaccgtaatttcactttccgcttcgctgggctcttcctcttcctcttgcgtccgcataccacgcgccactgg
gtcgtcttcattcagccgccgcactgtgcgcttacctcctttgccatgcttgattagcaccggtgggttgctgaaacccaccatttgtagcg
ccacatcttctctttcttcctcgctgtccacgattacctctggtgatggcgggcgctcgggcttgggagaagggcgcttctttttcttcttgg
gcgcaatggccaaatccgccgccgaggtcgatggccgcgggctgggtgtgcgcggcaccagcgcgtcttgtgatgagtcttcctcgt
cctcggactcgatacgccgcctcatccgcttttttgggggcgcccggggaggcggcggcgacggggacggggacgacacgtcctc
catggttgggggacgtcgcgccgcaccgcgtccgcgctcgggggtggtttcgcgctgctcctcttcccgactggccatttccttctcct
ataggcagaaaaagatcatggagtcagtcgagaagaaggacagcctaaccgccccctctgagttcgccaccaccgcctccaccgat
gccgccaacgcgcctaccaccttccccgtcgaggcacccccgcttgaggaggaggaagtgattatcgagcaggacccaggttttgta
agcgaagacgacgaggaccgctcagtaccaacagaggataaaaagcaagaccaggacaacgcagaggcaaacgaggaacaagt
cgggcggggggacgaaaggcatggcgactacctagatgtgggagacgacgtgctgttgaagcatctgcagcgccagtgcgccatt
atctgcgacgcgttgcaagagcgcagcgatgtgcccctcgccatagcggatgtcagccttgcctacgaacgccacctattctcaccgc
gcgtaccccccaaacgccaagaaaacggcacatgcgagcccaacccgcgcctcaacttctaccccgtatttgccgtgccagaggtg
cttgccacctatcacatctttttccaaaactgcaagatacccctatcctgccgtgccaaccgcagccgagcggacaagcagctggcctt
gcggcagggcgctgtcatacctgatatcgcctcgctcaacgaagtgccaaaaatctttgagggtcttggacgcgacgagaagcgcgc
ggcaaacgctctgcaacaggaaaacagcgaaaatgaaagtcactctggagtgttggtggaactcgagggtgacaacgcgcgcctag
ccgtactaaaacgcagcatcgaggtcacccactttgcctacccggcacttaacctaccccccaaggtcatgagcacagtcatgagtga
gctgatcgtgcgccgtgcgcagcccctggagagggatgcaaatttgcaagaacaaacagaggagggcctacccgcagttggcgac
gagcagctagcgcgctggcttcaaacgcgcgagcctgccgacttggaggagcgacgcaaactaatgatggccgcagtgctcgttac
cgtggagcttgagtgcatgcagcggttctttgctgacccggagatgcagcgcaagctagaggaaacattgcactacacctttcgacag
ggctacgtacgccaggcctgcaagatctccaacgtggagctctgcaacctggtctcctaccttggaattttgcacgaaaaccgccttgg
gcaaaacgtgcttcattccacgctcaagggcgaggcgcgccgcgactacgtccgcgactgcgtttacttatttctatgctacacctggc
agacggccatgggcgtttggcagcagtgcttggaggagtgcaacctcaaggagctgcagaaactgctaaagcaaaacttgaaggac
ctatggacggccttcaacgagcgctccgtggccgcgcacctggcggacatcattttccccgaacgcctgcttaaaaccctgcaacag
ggtctgccagacttcaccagtcaaagcatgttgcagaactttaggaactttatcctagagcgctcaggaatcttgcccgccacctgctgt
gcacttcctagcgactttgtgcccattaagtaccgcgaatgccctccgccgctttggggccactgctaccttctgcagctagccaactac
cttgcctaccactctgacataatggaagacgtgagcggtgacggtctactggagtgtcactgtcgctgcaacctatgcaccccgcacc
gctccctggtttgcaattcgcagctgcttaacgaaagtcaaattatcggtacctttgagctgcagggtccctcgcctgacgaaaagtccg
cggctccggggttgaaactcactccggggctgtggacgtcggcttaccttcgcaaatttgtacctgaggactaccacgcccacgagatt
aggttctacgaagaccaatcccgcccgccaaatgcggagcttaccgcctgcgtcattacccagggccacattcttggccaattgcaag
ccatcaacaaagcccgccaagagtttctgctacgaaagggacggggggtttacttggacccccagtccggcgaggagctcaaccca
atccccccgccgccgcagccctatcagcagcagccgcgggcccttgcttcccaggatggcacccaaaaagaagctgcagctgccg
ccgccacccacggacgaggaggaatactgggacagtcaggcagaggaggttttggacgaggaggaggaggacatgatggaaga
ctgggagagcctagacgaggaagcttccgaggtcgaagaggtgtcagacgaaacaccgtcaccctcggtcgcattcccctcgccgg
cgccccagaaatcggcaaccggttccagcatggctacaacctccgctcctcaggcgccgccggcactgcccgttcgccgacccaac
cgtagatgggacaccactggaaccagggccggtaagtccaagcagccgccgccgttagcccaagagcaacaacagcgccaaggc
taccgctcatggc
gcgggcacaagaacgccatagttgcttgcttgcaagactgtgggggcaacatctccttcgcccgccgctttcttctctaccatcacggc
gtggccttcccccgtaacatcctgcattactaccgtcatctctacagcccatactgcaccggcggcagcggcagcggcagcaacagc
agcggccacacagaagcaaaggcgaccggatagcaagactctgacaaagcccaagaaatccacagcggcggcagcagcaggag
gaggagcgctgcgtctggcgcccaacgaacccgtatcgacccgcgagcttagaaacaggatttttcccactctgtatgctatatttcaa
cagagcaggggccaagaacaagagctgaaaataaaaaacaggtctctgcgatccctcacccgcagctgcctgtatcacaaaagcga
agatcagcttcggcgcacgctggaagacgcggaggctctcttcagtaaatactgcgcgctgactcttaaggactagtttcgcgcccttt
ctcaaatttaagcgcgaaaactacgtcatctccagcggccacacccggcgccagcacctgtcgtcagcgccattatgagcaaggaaa
ttcccacgccctacatgtggagttaccagccacaaatgggacttgcggctggagctgcccaagactactcaacccgaataaactacat
gagcgcgggaccccacatgatatcccgggtcaacggaatccgcgcccaccgaaaccgaattctcttggaacaggcggctattacca
ccacacctcgtaataaccttaatccccgtagttggcccgctgccctggtgtaccaggaaagtcccgctcccaccactgtggtacttccca
gagacgcccaggccgaagttcagatgactaactcaggggcgcagcttgcgggcggctttcgtcacagggtgcggtcgcccgggca
gggtataactcacctgacaatcagagggcgaggtattcagctcaacgacgagtcggtgagctcctcgcttggtctccgtccggacgg
gacatttcagatcggcggcgccggccgtccttcattcacgcctcgtcaggcaatcctaactctgcagacctcgtcctctgagccgcgct
ctggaggcattggaactctgcaatttattgaggagtttgtgccatcggtctactttaaccccttctcgggacctcccggccactatccggat
caatttattcctaactttgacgcggtaaaggactcggcggacggctacgactgaatgttaagtggagaggcagagcaactgcgcctga
aacacctggtccactgtcgccgccacaagtgctttgcccgcgactccggtgagttttgctactttgaattgcccgaggatcatatcgagg
gcccggcgcacggcgtccggcttaccgcccagggagagcttgcccgtagcctgattcgggagtttacccagcgccccctgctagttg
agcgggacaggggaccctgtgttctcactgtgatttgcaactgtcctaaccttggattacatcaagatctttgttgccatctctgtgctgagt
ataataaatacagaaattaaaatatactggggctcctatcgccatcctgtaaacgccaccgtcttcacccgcccaagcaaaccaaggcg
aaccttacctggtacttttaacatctctccctctgtgatttacaacagtttcaacccagacggagtgagtctacgagagaacctctccgagc
tcagctactccatcagaaaaaacaccaccctccttacctgccgggaacgtacgagtgcgtcaccggccgctgcaccacacctaccgc
ctgaccgtaaaccagactttttccggacagacctcaataactctgtttaccagaacaggaggtgagcttagaaaacccttagggtattag
gccaaaggcgcagctactgtggggtttatgaacaattcaagcaactctacgggctattctaattcaggtttctctagaaatggacggaatt
attacagagcagcgcctgctagaaagacgcagggcagcggccgagcaacagcgcatgaatcaagagctccaagacatggttaactt
gcaccagtgcaaaaggggtatcttttgtctggtaaagcaggccaaagtcacctacgacagtaataccaccggacaccgccttagctac
aagttgccaaccaagcgtcagaaattggtggtcatggtgggagaaaagcccattaccataactcagcactcggtagaaaccgaaggc
tgcattcactcaccttgtcaaggacctgaggatctctgcacccttattaagaccctgtgcggtctcaaagatcttattccctttaactaataa
aaaaaaataataaagcatcacttacttaaaatcagttagcaaatttctgtccagtttattcagcagcacctccttgccctcctcccagctctg
gtattgcagcttcctcctggctgcaaactttctccacaatctaaatggaatgtcagtttcctcctgttcctgtccatccgcacccactatcttc
atgttgttgcagatgaagcgcgcaagaccgtctgaagataccttcaaccccgtgtatccatatgacacggaaaccggtcctccaactgt
gccttttcttactcctccctttgtatcccccaatgggtttcaagagagtccccctggggtactctctttgcgcctatccgaacctctagttacc
tccaatggcatgcttgcgctcaaaatgggcaacggcctctctctggacgaggccggcaaccttacctcccaaaatgtaaccactgtga
gcccacctctcaaaaaaaccaagtcaaacataaacctggaaatatctgcacccctcacagttacctcagaagccctaactgtggctgcc
gccgcacctctaatggtcgcgggcaacacactcaccatgcaatcacaggccccgctaaccgtgcacgactccaaacttagcattgcc
acccaaggacccctcacagtgtcagaaggaaagctagccctgcaaacatcaggccccctcaccaccaccgatagcagtacccttact
atcactgcctcaccccctctaactactgccactggtagcttgggcattgacttgaaagagcccatttatacacaaaatggaaaactagga
ctaaagtacggggctcctttgcatgtaacagacgacctaaacactttgaccgtagcaactggtccaggtgtgactattaataatacttcctt
gcaaactaaagttactggagccttgggttttgattcacaaggcaatatgcaacttaatgtagcaggaggactaaggattgattctcaaaac
agacgccttatacttgatgttagttatccgtttgatgctcaaaaccaactaaatctaagactaggacagggccctctttttataaactcagcc
cacaacttggatattaactacaacaaaggcctttacttgtttacagcttcaaacaattccaaaaagcttgaggttaacctaagcactgccaa
ggggttgatgtttgacgctacagccatagccattaatgcaggagatgggcttgaatttggttcacctaatgcaccaaacacaaatcccct
caaaacaaaaattggccatggcctagaatttgattcaaacaaggctatggttcctaaactaggaactggccttagttttgacagcacagg
tgccattacagtaggaaacaaaaataatgataagctaactttgtggaccacaccagctccatctcctaactgtagactaaatgcagagaa
agatgctaaactcactttggtcttaacaaaatgtggcagtcaaatacttgctacagtttcagttttggctgttaaaggcagtttggctccaat
atctggaacagttcaaag
tgctcatcttattataagatttgacgaaaatggagtgctactaaacaattccttcctggacccagaatattggaactttagaaatggagatct
tactgaaggcacagcctatacaaacgctgttggatttatgcctaacctatcagcttatccaaaatctcacggtaaaactgccaaaagtaac
attgtcagtcaagtttacttaaacggagacaaaactaaacctgtaacactaaccattacactaaacggtacacaggaaacaggagacac
aactccaagtgcatactctatgtcattttcatgggactggtctggccacaactacattaatgaaatatttgccacatcctcttacactttttcat
acattgcccaagaataaagaatcgtttgtgttatgtttcaacgtgtttatttttcaattgcagaaaatttcgaatcatttttcattcagtagtatag
ccccaccaccacatagcttatacagatcaccgtaccttaatcaaactcacagaaccctagtattcaacctgccacctccctcccaacaca
cagagtacacagtcctttctccccggctggccttaaaaagcatcatatcatgggtaacagacatattcttaggtgttatattccacacggttt
cctgtcgagccaaacgctcatcagtgatattaataaactccccgggcagctcacttaagttcatgtcgctgtccagctgctgagccacag
gctgctgtccaacttgcggttgcttaacgggcggcgaaggagaagtccacgcctacatgggggtagagtcataatcgtgcatcaggat
agggcggtggtgctgcagcagcgcgcgaataaactgctgccgccgccgctccgtcctgcaggaatacaacatggcagtggtctcct
cagcgatgattcgcaccgcccgcagcataaggcgccttgtcctccgggcacagcagcgcaccctgatctcacttaaatcagcacagt
aactgcagcacagcaccacaatattgttcaaaatcccacagtgcaaggcgctgtatccaaagctcatggcggggaccacagaaccca
cgtggccatcataccacaagcgcaggtagattaagtggcgacccctcataaacacgctggacataaacattacctcttttggcatgttgt
aattcaccacctcccggtaccatataaacctctgattaaacatggcgccatccaccaccatcctaaaccagctggccaaaacctgcccg
ccggctatacactgcagggaaccgggactggaacaatgacagtggagagcccaggactcgtaaccatggatcatcatgctcgtcatg
atatcaatgttggcacaacacaggcacacgtgcatacacttcctcaggattacaagctcctcccgcgttagaaccatatcccagggaac
aacccattcctgaatcagcgtaaatcccacactgcagggaagacctcgcacgtaactcacgttgtgcattgtcaaagtgttacattcggg
cagcagcggatgatcctccagtatggtagcgcgggtttctgtctcaaaaggaggtagacgatccctactgtacggagtgcgccgaga
caaccgagatcgtgttggtcgtagtgtcatgccaaatggaacgccggacgtagtcatatttcctgaagcaaaaccaggtgcgggcgtg
acaaacagatctgcgtctccggtctcgccgcttagatcgctctgtgtagtagttgtagtatatccactctctcaaagcatccaggcgcccc
ctggcttcgggttctatgtaaactccttcatgcgccgctgccctgataacatccaccaccgcagaataagccacacccagccaacctac
acattcgttctgcgagtcacacacgggaggagcgggaagagctggaagaaccatgtttttttttttattccaaaagattatccaaaacctc
aaaatgaagatctattaagtgaacgcgctcccctccggtggcgtggtcaaactctacagccaaagaacagataatggcatttgtaagat
gttgcacaatggcttccaaaaggcaaacggccctcacgtccaagtggacgtaaaggctaaacccttcagggtgaatctcctctataaac
attccagcaccttcaaccatgcccaaataattctcatctcgccaccttctcaatatatctctaagcaaatcccgaatattaagtccggccatt
gtaaaaatctgctccagagcgccctccaccttcagcctcaagcagcgaatcatgattgcaaaaattcaggttcctcacagacctgtataa
gattcaaaagcggaacattaacaaaaataccgcgatcccgtaggtcccttcgcagggccagctgaacataatcgtgcaggtctgcacg
gaccagcgcggccacttccccgccaggaaccttgacaaaagaacccacactgattatgacacgcatactcggagctatgctaaccag
cgtagccccgatgtaagctttgttgcatgggcggcgatataaaatgcaaggtgctgctcaaaaaatcaggcaaagcctcgcgcaaaaa
agaaagcacatcgtagtcatgctcatgcagataaaggcaggtaagctccggaaccaccacagaaaaagacaccatttttctctcaaac
atgtctgcgggtttctgcataaacacaaaataaaataacaaaaaaacatttaaacattagaagcctgtcttacaacaggaaaaacaaccc
ttataagcataagacggactacggccatgccggcgtgaccgtaaaaaaactggtcaccgtgattaaaaagcaccaccgacagctcct
cggtcatgtccggagtcataatgtaagactcggtaaacacatcaggttgattcacatcggtcagtgctaaaaagcgaccgaaatagccc
gggggaatacatacccgcaggcgtagagacaacattacagcccccataggaggtataacaaaattaataggagagaaaaacacata
aacacctgaaaaaccctcctgcctaggcaaaatagcaccctcccgctccagaacaacatacagcgcttccacagcggcagccataac
agtcagccttaccagtaaaaaagaaaacctattaaaaaaacaccactcgacacggcaccagctcaatcagtcacagtgtaaaaaagg
gccaagtgcagagcgagtatatataggactaaaaaatgacgtaacggttaaagtccacaaaaaacacccagaaaaccgcacgcgaa
cctacgcccagaaacgaaagccaaaaaacccacaacttcctcaaatcgtcacttccgttttcccacgttacgtcacttcccattttaagaa
aactacaattcccaacacatacaagttactccgccctaaaacctacgtcacccgccccgttcccacgccccgcgccacgtcacaaact
ccaccccctcattatcatattggcttcaatccaaaataaggtatattattgatgatgttaattaatttaaatccgcatgcgatatcgagctctcc
cgggaattcggatctgcgacgcgaggctggatggccttccccattatgattcttctcgcttccggcggcatcgggatgcccgcgttgca
ggccatgctgtccaggcaggtagatgacgaccatcagggacagcttcacggccagcaaaaggccaggaaccgtaaaaaggccgc
gttgctggcgtttttccataggctccgcccccctgacgagcatcacaaaaatcgacgctcaagtcagaggtggcgaaacccgacagg
actataaagataccaggcgtttccccctggaagctccctcgtgcgctctcctgttccgaccctgccgcttaccggatacctgtccgccttt
ctcccttcgggaagc
gtggcgctttctcaatgctcacgctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcgccactggcagcagccactgg
taacaggattagcagagcgaggtatgtaggcggtgctacagagttcttgaagtggtggcctaactacggctacactagaaggacagta
tttggtatctgcgctctgctgaagccagttaccttcggaaaaagagttggtagctcttgatccggcaaacaaaccaccgctggtagcggt
ggtttttttgtttgcaagcagcagattacgcgcagaaaaaaaggatctcaagaagatcctttgatcttttctacggggtctgacgctcagtg
gaacgaaaactcacgttaagggattttggtcatgagattatcaaaaaggatcttcacctagatccttttaaatcaatctaaagtatatatgag
taaacttggtctgacagttaccaatgcttaatcagtgaggcacctatctcagcgatctgtctatttcgttcatccatagttgcctgactcccc
gtcgtgtagataactacgatacgggagggcttaccatctggccccagtgctgcaatgataccgcgagacccacgctcaccggctcca
gatttatcagcaataaaccagccagccggaagggccgagcgcagaagtggtcctgcaactttatccgcctccatccagtctattaattgt
tgccgggaagctagagtaagtagttcgccagttaatagtttgcgcaacgttgttgccattgntgcaggcatcgtggtgtcacgctcgtcg
tttggtatggcttcattcagctccggttcccaacgatcaaggcgagttacatgatcccccatgttgtgcaaaaaagcggttagctccttcg
gtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaa
cacgggataataccgcgccacatagcagaactttaaaagtgctcatcattggaaaacgttcttcggggcgaaaactctcaaggatctta
ccgctgttgagatccagttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactcttcctttttcaatattattgaag
catttatcagggttattgtctcatgagcggatacatatttgaatgtatttagaaaaataaacaaataggggttccgcgcacatttccccgaa
aagtgccacctgacgtctaagaaaccattattatcatgacattaacctataaaaataggcgtatcacgaggccctttcgtcttcaaggatc
cgaattcccgggagagctcgatatcgcatgcggatttaaattaattaa

TABLE 7
Nucleotide sequence of pAd-GW-TO/tRNA.

catcatcaataatataccttattttggattgaagccaatatgataatgagggggtggagtttgtgacgtggcgcggggcgtgggaacgg
ggcgggtgacgtagtagtgtggcggaagtgtgatgttgcaagtgtggcggaacacatgtaagcgacggatgtggcaaaagtgacgtt
tttggtgtgcgccggtgtacacaggaagtgacaattttcgcgcggttttaggcggatgttgtagtaaatttgggcgtaaccgagtaagatt
tggccattttcgcgggaaaactgaataagaggaagtgaaatctgaataattttgtgttactcatagcgcgtaatatttgtctagggccgcg
gggactttgaccgtttacgtggagactcgcccaggtgtttttctcaggtgttttccgcgttccgggtcaaagttggcgttttattattatagtc
agtcgaagcttggatccggtacctctagaattctcgagcggccgctagcgacatcgatcacaagtttgtacaaaaaagcaggctttaaa
ggaaccaattcagtcgactctagaggatcgaaaccatcctctgctatatggccgcatatattttacttgaagactaggaccctacagaaa
aggggttttaaagtaggcgtgctaaacgtcagcggacctgacccgtgtaagaatccacaaggtatcctggtggaaatgcgcatttgtag
gcttcaatatctgtaatcctactaattaggtgtggagagctttcagccagtttcgtaggtttggagaccatttaggggttggcgtgtggccc
cctcgtaaagtctttcgtacttcctacatcagacaagtcttgcaatttgcaatatctcttttagccaatatctaaatctttaaaattttgattttgttt
tttacccaggatgagagacattccagagttgttaccttgtcaaaataaacaaatttaaagatgtctgtgaaaagaaacatatattcctcatg
ggaatatatccaggttgttgaaggaggtacgacctcgagatctctatcactgatagggagactcgagtgtagtcgtggccgagtggtta
aggcgatggactctaaatccattggggtctccccgcgcaggttcgaatcctgccgactacggcgtgctttttttactctcgggtagagga
aatccggtgcactacctgtgcaatcacacagaataacatggagtagtactttttattttcctgttattatctttctccataaaagtggaaccag
ataattttagttcttttgtgtaacaagactagagattttttgaagtgttacattggaaagcacttgaaaacacaagtaatttctgacactgctat
aaaaatgatggaaaaacgctcaagttgttttgcctttcagtcttcttgaaatgctgtctccctatctgaaatccagctcacgtctgacttccaa
aaccgtgcttgcctttaacttatggaataaatatctcaaacagatccccgggcgagctcgaattcgcggccgcactcgagatatctagac
ccagctttcttgtacaaagtggtgatcgattcgacagatcactgaaatgtgtgggcgtggcttaagggtgggaaagaatatataaggtgg
gggtcttatgtagttttgtatctgttttgcagcagccgccgccgccatgagcaccaactcgtttgatggaagcattgtgagctcatatttga
caacgcgcatgcccccatgggccggggtgcgtcagaatgtgatgggctccagcattgatggtcgccccgtcctgcccgcaaactcta
ctaccttgacctacgagaccgtgtctggaacgccgttggagactgcagcctccgccgccgcttcagccgctgcagccaccgcccgc
gggattgtgactgactttgctttcctgagcccgcttgcaagcagtgcagcttcccgttcatccgcccgcgatgacaagttgacggctcttt
tggcacaattggattctttgacccgggaacttaatgtcgtttctcagcagctgttggatctgcgccagcaggtttctgccctgaaggcttcc
tcccctcccaatgcggtttaaaacataaataaaaaaccagactctgtttggatttggatcaagcaagtgtcttgctgtctttatttaggggttt
tgcgcgcgcggtaggcccgggaccagcggtctcggtcgttgagggtcctgtgtattttttccaggacgtggtaaaggtgactctggatg
ttcagatacatgggcataagcccgtctctggggtggaggtagcaccactgcagagcttcatgctgcggggtggtgttgtagatgatcca
gtcgtagcaggagcgctgggcgtggtgcctaaaaatgtctttcagtagcaagctgattgccaggggcaggcccttggtgtaagtgttta
caaagcggttaagctgggatgggtgcatacgtggggatatgagatgcatcttggactgtatttttaggttggctatgttcccagccatatc
cctccggggattcatgttgtgcagaaccaccagcacagtgtatccggtgcacttgggaaatttgtcatgtagcttagaaggaaatgcgtg
gaagaacttggagacgcccttgtgacctccaagattttccatgcattcgtccataatgatggcaatgggcccacgggcggcggcctgg
gcgaagatatttctgggatcactaacgtcatagttgtgttccaggatgagatcgtcataggccatttttacaaagcgcgggcggagggtg
ccagactgcggtataatggttccatccggcccaggggcgtagttaccctcacagatttgcatttcccacgctttgagttcagatgggggg
atcatgtctacctgcggggcgatgaagaaaacggtttccggggtaggggagatcagctgggaagaaagcaggttcctgagcagctg
cgacttaccgcagccggtgggcccgtaaatcacacctattaccgggtgcaactggtagttaagagagctgcagctgccgtcatccctg
agcaggggggccacttcgttaagcatgtccctgactcgcatgttttccctgaccaaatccgccagaaggcgctcgccgcccagcgata
gcagttcttgcaaggaagcaaagtttttcaacggtttgagaccgtccgccgtaggcatgcttttgagcgtttgaccaagcagttccaggc
ggtcccacagctcggtcacctgctctacggcatctcgatccagcatatctcctcgtttcgcgggttggggcggctttcgctgtacggcag
tagtcggtgctcgtccagacgggccagggtcatgtctttccacgggcgcagggtcctcgtcagcgtagtctgggtcacggtgaaggg
gtgcgctccgggctgcgcgctggccagggtgcgcttgaggctggtcctgctggtgctgaagcgctgccggtcttcgccctgcgcgtc
ggccaggtagcatttgaccatggtgtcatagtccagcccctccgcggcgtggcccttggcgcgcagcttgcccttggaggaggcgcc
gcacgaggggcagtgcagacttttgagggcgtagagcttgggcgcgagaaataccgattccggggagtaggcatccgcgccgcag
gccccgcagacggtctcgcattccacgagccaggtgagctctggccgttcggggtcaaaaaccaggtttcccccatgctttttgatgcg
tttcttacctctggtttccatgagccggtgtccacgctcggtgacgaaaaggctgtccgtgtccccgtatacagacttgagaggcctgtcc
tcgagcggtgttccgcggtcctcctcgtatagaaactcggaccactctgagacaaaggctcgcgtccaggccagcacgaaggaggct
aagtgggagg
ggtagcggtcgttgtccactagggggtccactcgctccagggtgtgaagacacatgtcgccctcttcggcatcaaggaaggtgattgg
tttgtaggtgtaggccacgtgaccgggtgttcctgaaggggggctataaaagggggtgggggcgcgttcgtcctcactctcttccgcat
cgctgtctgcgagggccagctgttggggtgagtactccctctgaaaagcgggcatgacttctgcgctaagattgtcagtttccaaaaac
gaggaggatttgatattcacctggcccgcggtgatgcctttgagggtggccgcatccatctggtcagaaaagacaatctttttgttgtcaa
gcttggtggcaaacgacccgtagagggcgttggacagcaacttggcgatggagcgcagggtttggtttttgtcgcgatcggcgcgct
ccttggccgcgatgtttagctgcacgtattcgcgcgcaacgcaccgccattcgggaaagacggtggtgcgctcgtcgggcaccaggt
gcacgcgccaaccgcggttgtgcagggtgacaaggtcaacgctggtggctacctctccgcgtaggcgctcgttggtccagcagagg
cggccgcccttgcgcgagcagaatggcggtagggggtctagctgcgtctcgtccggggggtctgcgtccacggtaaagaccccgg
gcagcaggcgcgcgtcgaagtagtctatcttgcatccttgcaagtctagcgcctgctgccatgcgcgggcggcaagcgcgcgctcgt
atgggttgagtgggggaccccatggcatggggtgggtgagcgcggaggcgtacatgccgcaaatgtcgtaaacgtagaggggctct
ctgagtattccaagatatgtagggtagcatcttccaccgcggatgctggcgcgcacgtaatcgtatagttcgtgcgagggagcgagga
ggtcgggaccgaggttgctacgggcgggctgctctgctcggaagactatctgcctgaagatggcatgtgagttggatgatatggttgg
acgctggaagacgttgaagctggcgtctgtgagacctaccgcgtcacgcacgaaggaggcgtaggagtcgcgcagcttgttgacca
gctcggcggtgacctgcacgtctagggcgcagtagtccagggtttccttgatgatgtcatacttatcctgtcccttttttttccacagctcgc
ggttgaggacaaactcttcgcggtctttccagtactcttggatcggaaacccgtcggcctccgaacggtaagagcctagcatgtagaac
tggttgacggcctggtaggcgcagcatcccttttctacgggtagcgcgtatgcctgcgcggccttccggagcgaggtgtgggtgagc
gcaaaggtgtccctgaccatgactttgaggtactggtatttgaagtcagtgtcgtcgcatccgccctgctcccagagcaaaaagtccgt
gcgctttttggaacgcggatttggcagggcgaaggtgacatcgttgaagagtatctttcccgcgcgaggcataaagttgcgtgtgatgc
ggaagggtcccggcacctcggaacggttgttaattacctgggcggcgagcacgatctcgtcaaagccgttgatgttgtggcccacaat
gtaaagttccaagaagcgcgggatgcccttgatggaaggcaattttttaagttcctcgtaggtgagctcttcaggggagctgagcccgt
gctctgaaagggcccagtctgcaagatgagggttggaagcgacgaatgagctccacaggtcacgggccattagcatttgcaggtggt
cgcgaaaggtcctaaactggcgacctatggccattttttctggggtgatgcagtagaaggtaagcgggtcttgttcccagcggtcccat
ccaaggttcgcggctaggtctcgcgcggcagtcactagaggctcatctccgccgaacttcatgaccagcatgaagggcacgagctgc
ttcccaaaggcccccatccaagtataggtctctacatcgtaggtgacaaagagacgctcggtgcgaggatgcgagccgatcgggaag
aactggatctcccgccaccaattggaggagtggctattgatgtggtgaaagtagaagtccctgcgacgggccgaacactcgtgctgg
cttttgtaaaaacgtgcgcagtactggcagcggtgcacgggctgtacatcctgcacgaggttgacctgacgaccgcgcacaaggaag
cagagtgggaatttgagcccctcgcctggcgggtttggctggtggtcttctacttcggctgcttgtccttgaccgtctggctgctcgagg
ggagttacggtggatcggaccaccacgccgcgcgagcccaaagtccagatgtccgcgcgcggcggtcggagcttgatgacaacat
cgcgcagatgggagctgtccatggtctggagctcccgcggcgtcaggtcaggcgggagctcctgcaggtttacctcgcatagacgg
gtcagggcgcgggctagatccaggtgatacctaatttccaggggctggttggtggcggcgtcgatggcttgcaagaggccgcatccc
cgcggcgcgactacggtaccgcgcggcgggcggtgggccgcgggggtgtccttggatgatgcatctaaaagcggtgacgcgggc
gagcccccggaggtagggggggctccggacccgccgggagagggggcaggggcacgtcggcgccgcgcgcgggcaggagc
tggtgctgcgcgcgtaggttgctggcgaacgcgacgacgcggcggttgatctcctgaatctggcgcctctgcgtgaagacgacggg
cccggtgagcttgagcctgaaagagagttcgacagaatcaatttcggtgtcgttgacggcggcctggcgcaaaatctcctgcacgtct
cctgagttgtcttgataggcgatctcggccatgaactgctcgatctcttcctcctggagatctccgcgtccggctcgctccacggtggcg
gcgaggtcgttggaaatgcgggccatgagctgcgagaaggcgttgaggcctccctcgttccagacgcggctgtagaccacgccccc
ttcggcatcgcgggcgcgcatgaccacctgcgcgagattgagctccacgtgccgggcgaagacggcgtagtttcgcaggcgctgaa
agaggtagttgagggtggtggcggtgtgttctgccacgaagaagtacataacccagcgtcgcaacgtggattcgttgatatcccccaa
ggcctcaaggcgctccatggcctcgtagaagtccacggcgaagttgaaaaactgggagttgcgcgccgacacggttaactcctcctc
cagaagacggatgagctcggcgacagtgtcgcgcacctcgcgctcaaaggctacaggggcctcttcttcttcttcaatctcctcttccat
aagggcctccccttcttcttcttctggcggcggtgggggaggggggacacggcggcgacgacggcgcaccgggaggcggtcgac
aaagcgctcgatcatctccccgcggcgacggcgcatggtctcggtgacggcgcggccgttctcgcgggggcgcagttggaagacg
ccgcccgtcatgtcccggttatgggttggcggggggctgccatgcggcagggatacggcgctaacgatgcatctcaacaattgttgtg
taggtactccgccgccgagggacctgagcgagtccgcatcgaccggatcggaaaacctctcgagaaaggcgtctaaccagtcacag
tcgcaaggtagg
ctgagcaccgtggcgggcggcagcgggcggcggtcggggttgtttctggcggaggtgctgctgatgatgtaattaaagtaggcggt
cttgagacggcggatggtcgacagaagcaccatgtccttgggtccggcctgctgaatgcgcaggcggtcggccatgccccaggctt
cgttttgacatcggcgcaggtctttgtagtagtcttgcatgagcctttctaccggcacttcttcttctccttcctcttgtcctgcatctcttgcat
ctatcgctgcggcggcggcggagtttggccgtaggtggcgccctcttcctcccatgcgtgtgaccccgaagcccctcatcggctgaa
gcagggctaggtcggcgacaacgcgctcggctaatatggcctgctgcacctgcgtgagggtagactggaagtcatccatgtccacaa
agcggtggtatgcgcccgtgttgatggtgtaagtgcagttggccataacggaccagttaacggtctggtgacccggctgcgagagctc
ggtgtacctgagacgcgagtaagccctcgagtcaaatacgtagtcgttgcaagtccgcaccaggtactggtatcccaccaaaaagtgc
ggcggcggctggcggtagaggggccagcgtagggtggccggggctccgggggcgagatcttccaacataaggcgatgatatccg
tagatgtacctggacatccaggtgatgccggcggcggtggtggaggcgcgcggaaagtcgcggacgcggttccagatgttgcgca
gcggcaaaaagtgctccatggtcgggacgctctggccggtcaggcgcgcgcaatcgttgacgctctagaccgtgcaaaaggagag
cctgtaagcgggcactcttccgtggtctggtggataaattcgcaagggtatcatggcggacgaccggggttcgagccccgtatccggc
cgtccgccgtgatccatgcggttaccgcccgcgtgtcgaacccaggtgtgcgacgtcagacaacgggggagtgctccttttggcttcc
ttccaggcgcggcggctgctgcgctagcttttttggccactggccgcgcgcagcgtaagcggttaggctggaaagcgaaagcattaa
gtggctcgctccctgtagccggagggttattttccaagggttgagtcgcgggacccccggttcgagtctcggaccggccggactgcg
gcgaacgggggtttgcctccccgtcatgcaagaccccgcttgcaaattcctccggaaacagggacgagccccttttttgcttttcccag
atgcatccggtgctgcggcagatgcgcccccctcctcagcagcggcaagagcaagagcagcggcagacatgcagggcaccctcc
cctcctcctaccgcgtcaggaggggcgacatccgcggttgacgcggcagcagatggtgattacgaacccccgcggcgccgggccc
ggcactacctggacttggaggagggcgagggcctggcgcggctaggagcgccctctcctgagcggtacccaagggtgcagctga
agcgtgatacgcgtgaggcgtacgtgccgcggcagaacctgtttcgcgaccgcgagggagaggagcccgaggagatgcgggatc
gaaagttccacgcagggcgcgagctgcggcatggcctgaatcgcgagcggttgctgcgcgaggaggactttgagcccgacgcgc
gaaccgggattagtcccgcgcgcgcacacgtggcggccgccgacctggtaaccgcatacgagcagacggtgaaccaggagatta
actttcaaaaaagctttaacaaccacgtgcgtacgcttgtggcgcgcgaggaggtggctataggactgatgcatctgtgggactttgtaa
gcgcgctggagcaaaacccaaatagcaagccgctcatggcgcagctgttccttatagtgcagcacagcagggacaacgaggcattc
agggatgcgctgctaaacatagtagagcccgagggccgctggctgctcgatttgataaacatcctgcagagcatagtggtgcaggag
cgcagcttgagcctggctgacaaggtggccgccatcaactattccatgcttagcctgggcaagttttacgcccgcaagatataccatac
cccttacgttcccatagacaaggaggtaaagatcgaggggttctacatgcgcatggcgctgaaggtgcttaccttgagcgacgacctg
ggcgtttatcgcaacgagcgcatccacaaggccgtgagcgtgagccggcggcgcgagctcagcgaccgcgagctgatgcacagc
ctgcaaagggccctggctggcacgggcagcggcgatagagaggccgagtcctactttgacgcgggcgctgacctgcgctgggccc
caagccgacgcgccctggaggcagctggggccggacctgggctggcggtggcacccgcgcgcgctggcaacgtcggcggcgtg
gaggaatatgacgaggacgatgagtacgagccagaggacggcgagtactaagcggtgatgtttctgatcagatgatgcaagacgca
acggacccggcggtgcgggcggcgctgcagagccagccgtccggccttaactccacggacgactggcgccaggtcatggaccgc
atcatgtcgctgactgcgcgcaatcctgacgcgttccggcagcagccgcaggccaaccggctctccgcaattctggaagcggtggtc
ccggcgcgcgcaaaccccacgcacgagaaggtgctggcgatcgtaaacgcgctggccgaaaacagggccatccggcccgacga
ggccggcctggtctacgacgcgctgcttcagcgcgtggctcgttacaacagcggcaacgtgcagaccaacctggaccggctggtgg
gggatgtgcgcgaggccgtggcgcagcgtgagcgcgcgcagcagcagggcaacctgggctccatggttgcactaaacgccttcct
gagtacacagcccgccaacgtgccgcggggacaggaggactacaccaactttgtgagcgcactgcggctaatggtgactgagaca
ccgcaaagtgaggtgtaccagtctgggccagactattttttccagaccagtagacaaggcctgcagaccgtaaacctgagccaggctt
tcaaaaacttgcaggggctgtggggggtgcgggctcccacaggcgaccgcgcgaccgtgtctagcttgctgacgcccaactcgcgc
ctgttgctgctgctaatagcgcccttcacggacagtggcagcgtgtcccgggacacatacctaggtcacttgctgacactgtaccgcga
ggccataggtcaggcgcatgtggacgagcatactttccaggagattacaagtgtcagccgcgcgctggggcaggaggacacgggc
agcctggaggcaaccctaaactacctgctgaccaaccggcggcagaagatcccctcgttgcacagtttaaacagcgaggaggagcg
cattttgcgctacgtgcagcagagcgtgagccttaacctgatgcgcgacggggtaacgcccagcgtggcgctggacatgaccgcgc
gcaacatggaaccgggcatgtatgcctcaaaccggccgtttatcaaccgcctaatggactacttgcatcgcgcggccgccgtgaacc
ccgagtatttcaccaatgccatcttgaacccgcactggctaccgccccctggtttctacaccgggggattcgaggtgcccgagggtaa
cgatggattcctctggg
acgacatagacgacagcgtgttttccccgcaaccgcagaccctgctagagttgcaacagcgcgagcaggcagaggcggcgctgcg
aaaggaaagcttccgcaggccaagcagcttgtccgatctaggcgctgcggccccgcggtcagatgctagtagcccatttccaagctt
gatagggtctcttaccagcactcgcaccacccgcccgcgcctgctgggcgaggaggagtacctaaacaactcgctgctgcagccgc
agcgcgaaaaaaacctgcctccggcatttcccaacaacgggatagagagcctagtggacaagatgagtagatggaagacgtacgcg
caggagcacagggacgtgccaggcccgcgcccgcccacccgtcgtcaaaggcacgaccgtcagcggggtctggtgtgggagga
cgatgactcggcagacgacagcagcgtcctggatttgggagggagtggcaacccgtttgcgcaccttcgccccaggctggggagaa
tgttttaaaaaaaaaaaagcatgatgcaaaataaaaaactcaccaaggccatggcaccgagcgttggttttcttgtattccccttagtatgc
ggcgcgcggcgatgtatgaggaaggtcctcctccctcctacgagagtgtggtgagcgcggcgccagtggcggcggcgctgggttct
cccttcgatgctcccctggacccgccgtttgtgcctccgcggtacctgcggcctaccggggggagaaacagcatccgttactctgagtt
ggcacccctattcgacaccacccgtgtgtacctggtggacaacaagtcaacggatgtggcatccctgaactaccagaacgaccacag
caactttctgaccacggtcattcaaaacaatgactacagcccgggggaggcaagcacacagaccatcaatcttgacgaccggtcgca
ctggggcggcgacctgaaaaccatcctgcataccaacatgccaaatgtgaacgagttcatgtttaccaataagtttaaggcgcgggtg
atggtgtcgcgcttgcctactaaggacaatcaggtggagctgaaatacgagtgggtggagttcacgctgcccgagggcaactactcc
gagaccatgaccatagaccttatgaacaacgcgatcgtggagcactacttgaaagtgggcagacagaacggggttctggaaagcga
catcggggtaaagtttgacacccgcaacttcagactggggtttgaccccgtcactggtcttgtcatgcctggggtatatacaaacgaag
ccttccatccagacatcattttgctgccaggatgcggggtggacttcacccacagccgcctgagcaacttgttgggcatccgcaagcg
gcaacccttccaggagggctttaggatcacctacgatgatctggagggtggtaacattcccgcactgttggatgtggacgcctaccag
gcgagcttgaaagatgacaccgaacagggcgggggtggcgcaggcggcagcaacagcagtggcagcggcgcggaagagaact
ccaacgcggcagccgcggcaatgcagccggtggaggacatgaacgatcatgccattcgcggcgacacctttgccacacgggctga
ggagaagcgcgctgaggccgaagcagcggccgaagctgccgcccccgctgcgcaacccgaggtcgagaagcctcagaagaaa
ccggtgatcaaacccctgacagaggacagcaagaaacgcagttacaacctaataagcaatgacagcaccttcacccagtaccgcag
ctggtaccttgcatacaactacggcgaccctcagaccggaatccgctcatggaccctgctttgcactcctgacgtaacctgcggctcgg
agcaggtctactggtcgttgccagacatgatgcaagaccccgtgaccttccgctccacgcgccagatcagcaactttccggtggtggg
cgccgagctgttgcccgtgcactccaagagcttctacaacgaccaggccgtctactcccaactcatccgccagtttacctctctgaccc
acgtgttcaatcgctttcccgagaaccagattttggcgcgcccgccagcccccaccatcaccaccgtcagtgaaaacgttcctgctctc
acagatcacgggacgctaccgctgcgcaacagcatcggaggagtccagcgagtgaccattactgacgccagacgccgcacctgcc
cctacgtttacaaggccctgggcatagtctcgccgcgcgtcctatcgagccgcactttttgagcaagcatgtccatccttatatcgccca
gcaataacacaggctggggcctgcgcttcccaagcaagatgtttggcggggccaagaagcgctccgaccaacacccagtgcgcgt
gcgcgggcactaccgcgcgccctggggcgcgcacaaacgcggccgcactgggcgcaccaccgtcgatgacgccatcgacgcgg
tggtggaggaggcgcgcaactacacgcccacgccgccaccagtgtccacagtggacgcggccattcagaccgtggtgcgcggag
cccggcgctatgctaaaatgaagagacggcggaggcgcgtagcacgtcgccaccgccgccgacccggcactgccgcccaacgc
gcggcggcggccctgcttaaccgcgcacgtcgcaccggccgacgggcggccatgcgggccgctcgaaggctggccgcgggtatt
gtcactgtgccccccaggtccaggcgacgagcggccgccgcagcagccgcggccattagtgctatgactcagggtcgcaggggca
acgtgtattgggtgcgcgactcggttagcggcctgcgcgtgcccgtgcgcacccgccccccgcgcaactagattgcaagaaaaaact
acttagactcgtactgttgtatgtatccagcggcggcggcgcgcaacgaagctatgtccaagcgcaaaatcaaagaagagatgctcca
ggtcatcgcgccggagatctatggccccccgaagaaggaagagcaggattacaagccccgaaagctaaagcgggtcaaaaagaa
aaagaaagatgatgatgatgaacttgacgacgaggtggaactgctgcacgctaccgcgcccaggcgacgggtacagtggaaaggt
cgacgcgtaaaacgtgttttgcgacccggcaccaccgtagtctttacgcccggtgagcgctccacccgcacctacaagcgcgtgtatg
atgaggtgtacggcgacgaggacctgcttgagcaggccaacgagcgcctcggggagtttgcctacggaaagcggcataaggacat
gctggcgttgccgctggacgagggcaacccaacacctagcctaaagcccgtaacactgcagcaggtgctgcccgcgcttgcaccgt
ccgaagaaaagcgcggcctaaagcgcgagtctggtgacttggcacccaccgtgcagctgatggtacccaagcgccagcgactgga
agatgtcttggaaaaaatgaccgtggaacctgggctggagcccgaggtccgcgtgcggccaatcaagcaggtggcgccgggactg
ggcgtgcagaccgtggacgttcagatacccactaccagtagcaccagtattgccaccgccacagagggcatggagacacaaacgtc
cccggttgcctcagcggtggcggatgccgcggtgcaggcggtcgctgcggccgcgtccaagacctctacggaggtgcaaacggac
ccgtggatgtttcg
cgtttcagccccccggcgcccgcgcggttcgaggaagtacggcgccgccagcgcgctactgcccgaatatgccctacatccttccat
tgcgcctacccccggctatcgtggctacacctaccgccccagaagacgagcaactacccgacgccgaaccaccactggaacccgc
cgccgccgtcgccgtcgccagcccgtgctggccccgatttccgtgcgcagggtggctcgcgaaggaggcaggaccctggtgctgc
caacagcgcgctaccaccccagcatcgtttaaaagccggtctttgtggttcttgcagatatggccctcacctgccgcctccgtttcccgg
tgccgggattccgaggaagaatgcaccgtaggaggggcatggccggccacggcctgacgggcggcatgcgtcgtgcgcaccacc
ggcggcggcgcgcgtcgcaccgtcgcatgcgcggcggtatcctgcccctccttattccactgatcgccgcggcgattggcgccgtg
cccggaattgcatccgtggccttgcaggcgcagagacactgattaaaaacaagttgcatgtggaaaaatcaaaataaaaagtctggac
tctcacgctcgcttggtcctgtaactattttgtagaatggaagacatcaactttgcgtctctggccccgcgacacggctcgcgcccgttca
tgggaaactggcaagatatcggcaccagcaatatgagcggtggcgccttcagctggggctcgctgtggagcggcattaaaaatttcg
gttccaccgttaagaactatggcagcaaggcctggaacagcagcacaggccagatgctgagggataagttgaaagagcaaaatttcc
aacaaaaggtggtagatggcctggcctctggcattagcggggtggtggacctggccaaccaggcagtgcaaaataagattaacagta
agcttgatccccgccctcccgtagaggagcctccaccggccgtggagacagtgtctccagaggggcgtggcgaaaagcgtccgcg
ccccgacagggaagaaactctggtgacgcaaatagacgagcctccctcgtacgaggaggcactaaagcaaggcctgcccaccacc
cgtcccatcgcgcccatggctaccggagtgctgggccagcacacacccgtaacgctggacctgcctccccccgccgacacccagc
agaaacctgtgctgccaggcccgaccgccgttgttgtaacccgtcctagccgcgcgtccctgcgccgcgccgccagcggtccgcga
tcgttgcggcccgtagccagtggcaactggcaaagcacactgaacagcatcgtgggtctgggggtgcaatccctgaagcgccgacg
atgcttctgaatagctaacgtgtcgtatgtgtgtcatgtatgcgtccatgtcgccgccagaggagctgctgagccgccgcgcgcccgct
ttccaagatggctaccccttcgatgatgccgcagtggtcttacatgcacatctcgggccaggacgcctcggagtacctgagccccggg
ctggtgcagtttgcccgcgccaccgagacgtacttcagcctgaataacaagtttagaaaccccacggtggcgcctacgcacgacgtg
accacagaccggtcccagcgtttgacgctgcggttcatccctgtggaccgtgaggatactgcgtactcgtacaaggcgcggttcaccc
tagctgtgggtgataaccgtgtgctggacatggcttccacgtactttgacatccgcggcgtgctggacaggggccctacttttaagccct
actctggcactgcctacaacgccctggctcccaagggtgccccaaatccttgcgaatgggatgaagctgctactgctcttgaaataaac
ctagaagaagaggacgatgacaacgaagacgaagtagacgagcaagctgagcagcaaaaaactcacgtatttgggcaggcgcctt
attctggtataaatattacaaaggagggtattcaaataggtgtcgaaggtcaaacacctaaatatgccgataaaacatttcaacctgaacc
tcaaataggagaatctcagtggtacgaaactgaaattaatcatgcagctgggagagtccttaaaaagactaccccaatgaaaccatgtt
acggttcatatgcaaaacccacaaatgaaaatggagggcaaggcattcttgtaaagcaacaaaatggaaagctagaaagtcaagtgg
aaatgcaatttttctcaactactgaggcgaccgcaggcaatggtgataacttgactcctaaagtggtattgtacagtgaagatgtagatat
agaaaccccagacactcatatttcttacatgcccactattaaggaaggtaactcacgagaactaatgggccaacaatctatgcccaaca
ggcctaattacattgcttttagggacaattttattggtctaatgtattacaacagcacgggtaatatgggtgttctggcgggccaagcatcg
cagttgaatgctgttgtagatttgcaagacagaaacacagagctttcataccagcttttgcttgattccattggtgatagaaccaggtacttt
tctatgtggaatcaggctgttgacagctatgatccagatgttagaattattgaaaatcatggaactgaagatgaacttccaaattactgcttt
ccactgggaggtgtgattaatacagagactcttaccaaggtaaaacctaaaacaggtcaggaaaatggatgggaaaaagatgctaca
gaattttcagataaaaatgaaataagagttggaaataattttgccatggaaatcaatctaaatgccaacctgtggagaaatttcctgtactc
caacatagcgctgtatttgcccgacaagctaaagtacagtccttccaacgtaaaaatttctgataacccaaacacctacgactacatgaa
caagcgagtggtggctcccgggttagtggactgctacattaaccttggagcacgctggtcccttgactatatggacaacgtcaacccat
ttaaccaccaccgcaatgctggcctgcgctaccgctcaatgttgctgggcaatggtcgctatgtgcccttccacatccaggtgcctcag
aagttctttgccattaaaaacctccttctcctgccgggctcatacacctacgagtggaacttcaggaaggatgttaacatggttctgcaga
gctccctaggaaatgacctaagggttgacggagccagcattaagtttgatagcatttgcctttacgccaccttcttccccatggcccaca
acaccgcctccacgcttgaggccatgcttagaaacgacaccaacgaccagtcctttaacgactatctctccgccgccaacatgctctac
cctatacccgccaacgctaccaacgtgcccatatccatcccctcccgcaactgggcggctttccgcggctgggccttcacgcgcctta
agactaaggaaaccccatcactgggctcgggctacgacccttattacacctactctggctctataccctacctagatggaaccttttacct
caaccacacctttaagaaggtggccattacctttgactcttctgtcagctggcctggcaatgaccgcctgcttacccccaacgagtttgaa
attaagcgctcagttgacggggagggttacaacgttgcccagtgtaacatgaccaaagactggttcctggtacaaatgctagctaacta
caacattggctaccagggcttctatatcccagagagctacaaggaccgcatgtactccttctttagaaacttccagcccatgagccgtca
ggtggtggatgatacta
aatacaaggactaccaacaggtgggcatcctacaccaacacaacaactctggatttgttggctaccttgcccccaccatgcgcgaagg
acaggcctaccctgctaacttcccctatccgcttataggcaagaccgcagttgacagcattacccagaaaaagtttctttgcgatcgcac
cctttggcgcatcccattctccagtaactttatgtccatgggcgcactcacagacctgggccaaaaccttctctacgccaactccgccca
cgcgctagacatgacttttgaggtggatcccatggacgagcccacccttctttatgttttgtttgaagtctttgacgtggtccgtgtgcacc
ggccgcaccgcggcgtcatcgaaaccgtgtacctgcgcacgcccttctcggccggcaacgccacaacataaagaagcaagcaaca
tcaacaacagctgccgccatgggctccagtgagcaggaactgaaagccattgtcaaagatcttggttgtgggccatattttttgggcac
ctatgacaagcgctttccaggctttgtttctccacacaagctcgcctgcgccatagtcaatacggccggtcgcgagactgggggcgtac
actggatggcctttgcctggaacccgcactcaaaaacatgctacctctttgagccctttggcttttctgaccagcgactcaagcaggttta
ccagtttgagtacgagtcactcctgcgccgtagcgccattgcttcttcccccgaccgctgtataacgctggaaaagtccacccaaagcg
tacaggggcccaactcggccgcctgtggactattctgctgcatgtttctccacgcctttgccaactggccccaaactcccatggatcaca
accccaccatgaaccttattaccggggtacccaactccatgctcaacagtccccaggtacagcccaccctgcgtcgcaaccaggaac
agctctacagcttcctggagcgccactcgccctacttccgcagccacagtgcgcagattaggagcgccacttctttttgtcacttgaaaa
acatgtaaaaataatgtactagagacactttcaataaaggcaaatgcttttatttgtacactctcgggtgattatttacccccacccttgccgt
ctgcgccgtttaaaaatcaaaggggttctgccgcgcatcgctatgcgccactggcagggacacgttgcgatactggtgtttagtgctcc
acttaaactcaggcacaaccatccgcggcagctcggtgaagttttcactccacaggctgcgcaccatcaccaacgcgtttagcaggtc
gggcgccgatatcttgaagtcgcagttggggcctccgccctgcgcgcgcgagttgcgatacacagggttgcagcactggaacactat
cagcgccgggtggtgcacgctggccagcacgctcttgtcggagatcagatccgcgtccaggtcctccgcgttgctcagggcgaacg
gagtcaactttggtagctgccttcccaaaaagggcgcgtgcccaggctttgagttgcactcgcaccgtagtggcatcaaaaggtgacc
gtgcccggtctgggcgttaggatacagcgcctgcataaaagccttgatctgcttaaaagccacctgagcctttgcgccttcagagaaga
acatgccgcaagacttgccggaaaactgattggccggacaggccgcgtcgtgcacgcagcaccttgcgtcggtgttggagatctgca
ccacatttcggccccaccggttcttcacgatcttggccttgctagactgctccttcagcgcgcgctgcccgttttcgctcgtcacatccatt
tcaatcacgtgctccttatttatcataatgcttccgtgtagacacttaagctcgccttcgatctcagcgcagcggtgcagccacaacgcgc
agcccgtgggctcgtgatgcttgtaggtcacctctgcaaacgactgcaggtacgcctgcaggaatcgccccatcatcgtcacaaaggt
cttgttgctggtgaaggtcagctgcaacccgcggtgctcctcgttcagccaggtcttgcatacggccgccagagcttccacttggtcag
gcagtagtttgaagttcgcctttagatcgttatccacgtggtacttgtccatcagcgcgcgcgcagcctccatgcccttctcccacgcaga
cacgatcggcacactcagcgggttcatcaccgtaatttcactttccgcttcgctgggctcttcctcttcctcttgcgtccgcataccacgcg
ccactgggtcgtcttcattcagccgccgcactgtgcgcttacctcctttgccatgcttgattagcaccggtgggttgctgaaacccaccat
ttgtagcgccacatcttctctttcttcctcgctgtccacgattacctctggtgatggcgggcgctcgggcttgggagaagggcgcttcttttt
cttcttgggcgcaatggccaaatccgccgccgaggtcgatggccgcgggctgggtgtgcgcggcaccagcgcgtcttgtgatgagt
cttcctcgtcctcggactcgatacgccgcctcatccgcttttttgggggcgcccggggaggcggcggcgacggggacggggacgac
acgtcctccatggttgggggacgtcgcgccgcaccgcgtccgcgctcgggggtggtttcgcgctgctcctcttcccgactggccattt
ccttctcctataggcagaaaaagatcatggagtcagtcgagaagaaggacagcctaaccgccccctctgagttcgccaccaccgcct
ccaccgatgccgccaacgcgcctaccaccttccccgtcgaggcacccccgcttgaggaggaggaagtgattatcgagcaggaccc
aggttttgtaagcgaagacgacgaggaccgctcagtaccaacagaggataaaaagcaagaccaggacaacgcagaggcaaacga
ggaacaagtcgggcggggggacgaaaggcatggcgactacctagatgtgggagacgacgtgctgttgaagcatctgcagcgcca
gtgcgccattatctgcgacgcgttgcaagagcgcagcgatgtgcccctcgccatagcggatgtcagccttgcctacgaacgccaccta
ttctcaccgcgcgtaccccccaaacgccaagaaaacggcacatgcgagcccaacccgcgcctcaacttctaccccgtatttgccgtg
ccagaggtgcttgccacctatcacatctttttccaaaactgcaagatacccctatcctgccgtgccaaccgcagccgagcggacaagc
agctggccttgcggcagggcgctgtcatacctgatatcgcctcgctcaacgaagtgccaaaaatctttgagggtcttggacgcgacga
gaagcgcgcggcaaacgctctgcaacaggaaaacagcgaaaatgaaagtcactctggagtgttggtggaactcgagggtgacaac
gcgcgcctagccgtactaaaacgcagcatcgaggtcacccactttgcctacccggcacttaacctaccccccaaggtcatgagcaca
gtcatgagtgagctgatcgtgcgccgtgcgcagcccctggagagggatgcaaatttgcaagaacaaacagaggagggcctacccg
cagttggcgacgagcagctagcgcgctggcttcaaacgcgcgagcctgccgacttggaggagcgacgcaaactaatgatggccgc
agtgctcgttaccgtggagcttgagtgcatgcagcggttctttgctgacccggagatgcagcgcaagctagaggaaacattgcactac
acctttcgacagggctacg
tacgccaggcctgcaagatctccaacgtggagctctgcaacctggtctcctaccttggaattttgcacgaaaaccgccttgggcaaaac
gtgcttcattccacgctcaagggcgaggcgcgccgcgactacgtccgcgactgcgtttacttatttctatgctacacctggcagacggc
catgggcgtttggcagcagtgcttggaggagtgcaacctcaaggagctgcagaaactgctaaagcaaaacttgaaggacctatggac
ggccttcaacgagcgctccgtggccgcgcacctggcggacatcattttccccgaacgcctgcttaaaaccctgcaacagggtctgcc
agacttcaccagtcaaagcatgttgcagaactttaggaactttatcctagagcgctcaggaatcttgcccgccacctgctgtgcacttcct
agcgactttgtgcccattaagtaccgcgaatgccctccgccgctttggggccactgctaccttctgcagctagccaactaccttgcctac
cactctgacataatggaagacgtgagcggtgacggtctactggagtgtcactgtcgctgcaacctatgcaccccgcaccgctccctgg
tttgcaattcgcagctgcttaacgaaagtcaaattatcggtacctttgagctgcagggtccctcgcctgacgaaaagtccgcggctccgg
ggttgaaactcactccggggctgtggacgtcggcttaccttcgcaaatttgtacctgaggactaccacgcccacgagattaggttctac
gaagaccaatcccgcccgccaaatgcggagcttaccgcctgcgtcattacccagggccacattcttggccaattgcaagccatcaaca
aagcccgccaagagtttctgctacgaaagggacggggggtttacttggacccccagtccggcgaggagctcaacccaatccccccg
ccgccgcagccctatcagcagcagccgcgggcccttgcttcccaggatggcacccaaaaagaagctgcagctgccgccgccaccc
acggacgaggaggaatactgggacagtcaggcagaggaggttttggacgaggaggaggaggacatgatggaagactgggagag
cctagacgaggaagcttccgaggtcgaagaggtgtcagacgaaacaccgtcaccctcggtcgcattcccctcgccggcgccccaga
aatcggcaaccggttccagcatggctacaacctccgctcctcaggcgccgccggcactgcccgttcgccgacccaaccgtagatgg
gacaccactggaaccagggccggtaagtccaagcagccgccgccgttagcccaagagcaacaacagcgccaaggctaccgctca
tggcgcgggcacaagaacgccatagttgcttgcttgcaagactgtgggggcaacatctccttcgcccgccgctttcttctctaccatcac
ggcgtggccttcccccgtaacatcctgcattactaccgtcatctctacagcccatactgcaccggcggcagcggcagcggcagcaac
agcagcggccacacagaagcaaaggcgaccggatagcaagactctgacaaagcccaagaaatccacagcggcggcagcagcag
gaggaggagcgctgcgtctggcgcccaacgaacccgtatcgacccgcgagcttagaaacaggatttttcccactctgtatgctatattt
caacagagcaggggccaagaacaagagctgaaaataaaaaacaggtctctgcgatccctcacccgcagctgcctgtatcacaaaag
cgaagatcagcttcggcgcacgctggaagacgcggaggctctcttcagtaaatactgcgcgctgactcttaaggactagtttcgcgcc
ctttctcaaatttaagcgcgaaaactacgtcatctccagcggccacacccggcgccagcacctgtcgtcagcgccattatgagcaagg
aaattcccacgccctacatgtggagttaccagccacaaatgggacttgcggctggagctgcccaagactactcaacccgaataaacta
catgagcgcgggaccccacatgatatcccgggtcaacggaatccgcgcccaccgaaaccgaattctcttggaacaggcggctattac
caccacacctcgtaataaccttaatccccgtagttggcccgctgccctggtgtaccaggaaagtcccgctcccaccactgtggtacttcc
cagagacgcccaggccgaagttcagatgactaactcaggggcgcagcttgcgggcggctttcgtcacagggtgcggtcgcccggg
cagggtataactcacctgacaatcagagggcgaggtattcagctcaacgacgagtcggtgagctcctcgcttggtctccgtccggacg
ggacatttcagatcggcggcgccggccgtccttcattcacgcctcgtcaggcaatcctaactctgcagacctcgtcctctgagccgcgc
tctggaggcattggaactctgcaatttattgaggagtttgtgccatcggtctactttaaccccttctcgggacctcccggccactatccgga
tcaatttattcctaactttgacgcggtaaaggactcggcggacggctacgactgaatgttaagtggagaggcagagcaactgcgcctg
aaacacctggtccactgtcgccgccacaagtgctttgcccgcgactccggtgagttttgctactttgaattgcccgaggatcatatcgag
ggcccggcgcacggcgtccggcttaccgcccagggagagcttgcccgtagcctgattcgggagtttacccagcgccccctgctagtt
gagcgggacaggggaccctgtgttctcactgtgatttgcaactgtcctaaccttggattacatcaagatctttgttgccatctctgtgctga
gtataataaatacagaaattaaaatatactggggctcctatcgccatcctgtaaacgccaccgtcttcacccgcccaagcaaaccaagg
cgaaccttacctggtacttttaacatctctccctctgtgatttacaacagtttcaacccagacggagtgagtctacgagagaacctctccga
gctcagctactccatcagaaaaaacaccaccctccttacctgccgggaacgtacgagtgcgtcaccggccgctgcaccacacctacc
gcctgaccgtaaaccagactttttccggacagacctcaataactctgtttaccagaacaggaggtgagcttagaaaacccttagggtatt
aggccaaaggcgcagctactgtggggtttatgaacaattcaagcaactctacgggctattctaattcaggtttctctagaaatggacgga
attattacagagcagcgcctgctagaaagacgcagggcagcggccgagcaacagcgcatgaatcaagagctccaagacatggttaa
cttgcaccagtgcaaaaggggtatcttttgtctggtaaagcaggccaaagtcacctacgacagtaataccaccggacaccgccttagct
acaagttgccaaccaagcgtcagaaattggtggtcatggtgggagaaaagcccattaccataactcagcactcggtagaaaccgaag
gctgcattcactcaccttgtcaaggacctgaggatctctgcacccttattaagaccctgtgcggtctcaaagatcttattccctttaactaat
aaaaaaaaataataaagcatcacttacttaaaatcagttagcaaatttctgtccagtttattcagcagcacctccttgccctcctcccagctc
tggtattgca
gcttcctcctggctgcaaactttctccacaatctaaatggaatgtcagtttcctcctgttcctgtccatccgcacccactatcttcatgttgttg
cagatgaagcgcgcaagaccgtctgaagataccttcaaccccgtgtatccatatgacacggaaaccggtcctccaactgtgccttttctt
actcctccctttgtatcccccaatgggtttcaagagagtccccctggggtactctctttgcgcctatccgaacctctagttacctccaatgg
catgcttgcgctcaaaatgggcaacggcctctctctggacgaggccggcaaccttacctcccaaaatgtaaccactgtgagcccacct
ctcaaaaaaaccaagtcaaacataaacctggaaatatctgcacccctcacagttacctcagaagccctaactgtggctgccgccgcac
ctctaatggtcgcgggcaacacactcaccatgcaatcacaggccccgctaaccgtgcacgactccaaacttagcattgccacccaag
gacccctcacagtgtcagaaggaaagctagccctgcaaacatcaggccccctcaccaccaccgatagcagtacccttactatcactgc
ctcaccccctctaactactgccactggtagcttgggcattgacttgaaagagcccatttatacacaaaatggaaaactaggactaaagta
cggggctcctttgcatgtaacagacgacctaaacactttgaccgtagcaactggtccaggtgtgactattaataatacttccttgcaaact
aaagttactggagccttgggttttgattcacaaggcaatatgcaacttaatgtagcaggaggactaaggattgattctcaaaacagacgc
cttatacttgatgttagttatccgtttgatgctcaaaaccaactaaatctaagactaggacagggccctctttttataaactcagcccacaac
ttggatattaactacaacaaaggcctttacttgtttacagcttcaaacaattccaaaaagcttgaggttaacctaagcactgccaaggggtt
gatgtttgacgctacagccatagccattaatgcaggagatgggcttgaatttggttcacctaatgcaccaaacacaaatcccctcaaaac
aaaaattggccatggcctagaatttgattcaaacaaggctatggttcctaaactaggaactggccttagttttgacagcacaggtgccatt
acagtaggaaacaaaaataatgataagctaactttgtggaccacaccagctccatctcctaactgtagactaaatgcagagaaagatgc
taaactcactttggtcttaacaaaatgtggcagtcaaatacttgctacagtttcagttttggctgttaaaggcagtttggctccaatatctgga
acagttcaaagtgctcatcttattataagatttgacgaaaatggagtgctactaaacaattccttcctggacccagaatattggaactttaga
aatggagatcttactgaaggcacagcctatacaaacgctgttggatttatgcctaacctatcagcttatccaaaatctcacggtaaaactg
ccaaaagtaacattgtcagtcaagtttacttaaacggagacaaaactaaacctgtaacactaaccattacactaaacggtacacaggaa
acaggagacacaactccaagtgcatactctatgtcattttcatgggactggtctggccacaactacattaatgaaatatttgccacatcctc
ttacactttttcatacattgcccaagaataaagaatcgtttgtgttatgtttcaacgtgtttatttttcaattgcagaaaatttcgaatcatttttcat
tcagtagtatagccccaccaccacatagcttatacagatcaccgtaccttaatcaaactcacagaaccctagtattcaacctgccacctcc
ctcccaacacacagagtacacagtcctttctccccggctggccttaaaaagcatcatatcatgggtaacagacatattcttaggtgttatat
tccacacggtttcctgtcgagccaaacgctcatcagtgatattaataaactccccgggcagctcacttaagttcatgtcgctgtccagctg
ctgagccacaggctgctgtccaacttgcggttgcttaacgggcggcgaaggagaagtccacgcctacatgggggtagagtcataatc
gtgcatcaggatagggcggtggtgctgcagcagcgcgcgaataaactgctgccgccgccgctccgtcctgcaggaatacaacatgg
cagtggtctcctcagcgatgattcgcaccgcccgcagcataaggcgccttgtcctccgggcacagcagcgcaccctgatctcacttaa
atcagcacagtaactgcagcacagcaccacaatattgttcaaaatcccacagtgcaaggcgctgtatccaaagctcatggcggggac
cacagaacccacgtggccatcataccacaagcgcaggtagattaagtggcgacccctcataaacacgctggacataaacattacctct
tttggcatgttgtaattcaccacctcccggtaccatataaacctctgattaaacatggcgccatccaccaccatcctaaaccagctggcca
aaacctgcccgccggctatacactgcagggaaccgggactggaacaatgacagtggagagcccaggactcgtaaccatggatcatc
atgctcgtcatgatatcaatgttggcacaacacaggcacacgtgcatacacttcctcaggattacaagctcctcccgcgttagaaccata
tcccagggaacaacccattcctgaatcagcgtaaatcccacactgcagggaagacctcgcacgtaactcacgttgtgcattgtcaaagt
gttacattcgggcagcagcggatgatcctccagtatggtagcgcgggtttctgtctcaaaaggaggtagacgatccctactgtacggag
tgcgccgagacaaccgagatcgtgttggtcgtagtgtcatgccaaatggaacgccggacgtagtcatatttcctgaagcaaaaccagg
tgcgggcgtgacaaacagatctgcgtctccggtctcgccgcttagatcgctctgtgtagtagttgtagtatatccactctctcaaagcatc
caggcgccccctggcttcgggttctatgtaaactccttcatgcgccgctgccctgataacatccaccaccgcagaataagccacaccc
agccaacctacacattcgttctgcgagtcacacacgggaggagcgggaagagctggaagaaccatgtttttttttttattccaaaagatta
tccaaaacctcaaaatgaagatctattaagtgaacgcgctcccctccggtggcgtggtcaaactctacagccaaagaacagataatgg
catttgtaagatgttgcacaatggcttccaaaaggcaaacggccctcacgtccaagtggacgtaaaggctaaacccttcagggtgaatc
tcctctataaacattccagcaccttcaaccatgcccaaataattctcatctcgccaccttctcaatatatctctaagcaaatcccgaatattaa
gtccggccattgtaaaaatctgctccagagcgccctccaccttcagcctcaagcagcgaatcatgattgcaaaaattcaggttcctcaca
gacctgtataagattcaaaagcggaacattaacaaaaataccgcgatcccgtaggtcccttcgcagggccagctgaacataatcgtgc
aggtctgcacggaccagcgcggccacttccccgccaggaaccttgacaaaagaacccacactgattatgacacgcatactcggagct
atgctaaccag
cgtagccccgatgtaagctttgttgcatgggcggcgatataaaatgcaaggtgctgctcaaaaaatcaggcaaagcctcgcgcaaaaa
agaaagcacatcgtagtcatgctcatgcagataaaggcaggtaagctccggaaccaccacagaaaaagacaccatttttctctcaaac
atgtctgcgggtttctgcataaacacaaaataaaataacaaaaaaacatttaaacattagaagcctgtcttacaacaggaaaaacaaccc
ttataagcataagacggactacggccatgccggcgtgaccgtaaaaaaactggtcaccgtgattaaaaagcaccaccgacagctcct
cggtcatgtccggagtcataatgtaagactcggtaaacacatcaggttgattcacatcggtcagtgctaaaaagcgaccgaaatagccc
gggggaatacatacccgcaggcgtagagacaacattacagcccccataggaggtataacaaaattaataggagagaaaaacacata
aacacctgaaaaaccctcctgcctaggcaaaatagcaccctcccgctccagaacaacatacagcgcttccacagcggcagccataac
agtcagccttaccagtaaaaaagaaaacctattaaaaaaacaccactcgacacggcaccagctcaatcagtcacagtgtaaaaaagg
gccaagtgcagagcgagtatatataggactaaaaaatgacgtaacggttaaagtccacaaaaaacacccagaaaaccgcacgcgaa
cctacgcccagaaacgaaagccaaaaaacccacaacttcctcaaatcgtcacttccgttttcccacgttacgtcacttcccattttaagaa
aactacaattcccaacacatacaagttactccgccctaaaacctacgtcacccgccccgttcccacgccccgcgccacgtcacaaact
ccaccccctcattatcatattggcttcaatccaaaataaggtatattattgatgatgttaattaatttaaatccgcatgcgatatcgagctctcc
cgggaattcggatctgcgacgcgaggctggatggccttccccattatgattcttctcgcttccggcggcatcgggatgcccgcgttgca
ggccatgctgtccaggcaggtagatgacgaccatcagggacagcttcacggccagcaaaaggccaggaaccgtaaaaaggccgc
gttgctggcgtttttccataggctccgcccccctgacgagcatcacaaaaatcgacgctcaagtcagaggtggcgaaacccgacagg
actataaagataccaggcgtttccccctggaagctccctcgtgcgctctcctgttccgaccctgccgcttaccggatacctgtccgccttt
ctcccttcgggaagcgtggcgctttctcaatgctcacgctgtaggtatctcagttcggtgtaggtcgttcgctccaagctgggctgtgtgc
acgaaccccccgttcagcccgaccgctgcgccttatccggtaactatcgtcttgagtccaacccggtaagacacgacttatcgccactg
gcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaacc
accgctggtagcggtggtttttttgtttgcaagcagcagattacgcgcagaaaaaaaggatctcaagaagatcctttgatcttttctacgg
ggtctgacgctcagtggaacgaaaactcacgttaagggattttggtcatgagattatcaaaaaggatcttcacctagatccttttaaatcaa
tctaaagtatatatgagtaaacttggtctgacagttaccaatgcttaatcagtgaggcacctatctcagcgatctgtctatttcgttcatccat
agttgcctgactccccgtcgtgtagataactacgatacgggagggcttaccatctggccccagtgctgcaatgataccgcgagaccca
cgctcaccggctccagatttatcagcaataaaccagccagccggaagggccgagcgcagaagtggtcctgcaactttatccgcctcc
atccagtctattaattgttgccgggaagctagagtaagtagttcgccagttaatagtttgcgcaacgttgttgccattgntgcaggcatcgt
ggtgtcacgctcgtcgtttggtatggcttcattcagctccggttcccaacgatcaaggcgagttacatgatcccccatgttgtgcaaaaaa
gcggttagctccttcggtcctccgatcgttgtcagaagtaagttggccgcagtgttatcactcatggttatggcagcactgcataattctctt
actgtcatgccatccgtaagatgcttttctgtgactggtgagtactcaaccaagtcattctgagaatagtgtatgcggcgaccgagttgct
cttgcccggcgtcaacacgggataataccgcgccacatagcagaactttaaaagtgctcatcattggaaaacgttcttcggggcgaaa
actctcaaggatcttaccgctgttgagatccagttcgatgtaacccactcgtgcacccaactgatcttcagcatcttttactttcaccagcgt
ttctgggtgagcaaaaacaggaaggcaaaatgccgcaaaaaagggaataagggcgacacggaaatgttgaatactcatactcttcctt
tttcaatattattgaagcatttatcagggttattgtctcatgagcggatacatatttgaatgtatttagaaaaataaacaaataggggttccgc
gcacatttccccgaaaagtgccacctgacgtctaagaaaccattattatcatgacattaacctataaaaataggcgtatcacgaggccctt
tcgtcttcaaggatccgaattcccgggagagctcgatatcgcatgcggatttaaattaattaa

TABLE 8
Nucleotide sequence of pAdeno TAG tRNA.

| | |
|---|---|
| 1 | catcatcaat aatatacctt attttggatt gaagccaata tgataatgag ggggtggagt |
| 61 | ttgtgacgtg gcgcggggcg tgggaacggg gcgggtgacg tagtagtgtg gcggaagtgt |
| 121 | gatgttgcaa gtgtggcgga acacatgtaa gcgacggatg tggcaaaagt gacgtttttg |
| 181 | gtgtgcgccg gtgtacacag gaagtgacaa ttttcgcgcg gttttaggcg gatgttgtag |
| 241 | taaatttggg cgtaaccgag taagatttgg ccattttcgc gggaaaactg aataagagga |
| 301 | agtgaaatct gaataatttt gtgttactca tagcgcgtaa tatttgtcta gggccgcggg |
| 361 | gactttgacc gtttacgtgg agactcgccc aggtgttttt ctcaggtgtt ttccgcgttc |
| 421 | cgggtcaaag ttggcgtttt attattatag tcagtcgaag cttggatccg gtacctctag |
| 481 | aattctcgag cggccgctag cgacatcgat cacaagtttg tacaaaaaag caggctttaa |
| 541 | aggaaccaat tcagtcgact ctagaggatc gaaaccatcc tctgctatat ggccgcatat |
| 601 | attttacttg aagactagga ccctacagaa aaggggtttt aaagtaggcg tgctaaacgt |
| 661 | cagcggacct gacccgtgta agaatccaca aggtatcctg gtggaaatgc gcatttgtag |
| 721 | gcttcaatat ctgtaatcct actaattagg tgtggagagc tttcagccag tttcgtaggt |
| 781 | ttggagacca tttaggggtt ggcgtgtggc cccctcgtaa agtctttcgt acttcctaca |
| 841 | tcagacaagt cttgcaattt gcaatatctc ttttagccaa tatctaaatc tttaaaattt |
| 901 | tgattttgtt ttttacccag gatgagagac attccagagt tgttaccttg tcaaaataaa |
| 961 | caaatttaaa gatgtctgtg aaaagaaaca tatattcctc atgggaatat atccaggttg |
| 1021 | ttgaaggagg tacgacctcg agatctctat cactgatagg gagactcgag tgtagtcgtg |
| 1081 | gccgagtggt taaggcgatg gactctaaat ccattggggt ctccccgcgc aggttcgaat |
| 1141 | cctgccgact acggcgtgct ttttttactc tcgggtagag gaaatccggt gcactacctg |
| 1201 | tgcaatcaca cagaataaca tggagtagta ctttttattt tcctgttatt atctttctcc |
| 1261 | ataaaagtgg aaccagataa ttttagttct tttgtgtaac aagactagag attttttgaa |
| 1321 | gtgttacatt ggaaagcact tgaaaacaca agtaatttct gacactgcta taaaaatgat |
| 1381 | ggaaaaacgc tcaagttgtt ttgcctttca gtcttcttga aatgctgtct ccctatctga |
| 1441 | aatccagctc acgtctgact tccaaaaccg tgcttgcctt taacttatgg aataaatatc |
| 1501 | tcaaacagat ccccgggcga gctcgaattc gcggccgcac tcgagatatc tagacccagc |
| 1561 | tttcttgtac aaagtggtga tcgattcgac agatcactga aatgtgtggg cgtggcttaa |
| 1621 | gggtgggaaa gaatatataa ggtgggggtc ttatgtagtt ttgtatctgt tttgcagcag |
| 1681 | ccgccgccgc catgagcacc aactcgtttg atggaagcat tgtgagctca tatttgacaa |
| 1741 | cgcgcatgcc cccatgggcc ggggtgcgtc agaatgtgat gggctccagc attgatggtc |
| 1801 | gccccgtcct gcccgcaaac tctactacct tgacctacga gaccgtgtct ggaacgccgt |
| 1861 | tggagactgc agcctccgcc gccgcttcag ccgctgcagc caccgcccgc gggattgtga |
| 1921 | ctgactttgc tttcctgagc ccgcttgcaa gcagtgcagc ttcccgttca tccgcccgcg |
| 1981 | atgacaagtt gacggctctt ttggcacaat tggattcttt gacccgggaa cttaatgtcg |
| 2041 | tttctcagca gctgttggat ctgcgccagc aggtttctgc cctgaaggct tcctcccctc |
| 2101 | ccaatgcggt ttaaaacata aataaaaaac cagactctgt ttggatttgg atcaagcaag |
| 2161 | tgtcttgctg tctttattta ggggttttgc gcgcgcggta ggcccgggac cagcggtctc |
| 2221 | ggtcgttgag ggtcctgtgt attttttcca ggacgtggta aaggtgactc tggatgttca |
| 2281 | gatacatggg cataagcccg tctctggggt ggaggtagca ccactgcaga gcttcatgct |
| 2341 | gcggggtggt gttgtagatg atccagtcgt agcaggagcg ctgggcgtgg tgcctaaaaa |
| 2401 | tgtctttcag tagcaagctg attgccaggg gcaggccctt ggtgtaagtg tttacaaagc |
| 2461 | ggttaagctg ggatgggtgc atacgtgggg atatgagatg catcttggac tgtattttta |
| 2521 | ggttggctat gttcccagcc atatccctcc ggggattcat gttgtgcaga accaccagca |
| 2581 | cagtgtatcc ggtgcacttg ggaaatttgt catgtagctt agaaggaaat gcgtggaaga |
| 2641 | acttggagac gcccttgtga cctccaagat tttccatgca ttcgtccata atgatggcaa |
| 2701 | tgggcccacg ggcggcggcc tgggcgaaga tatttctggg atcactaacg tcatagttgt |
| 2761 | gttccaggat gagatcgtca taggccattt ttacaaagcg cgggcggagg gtgccagact |
| 2821 | gcggtataat ggttccatcc ggcccagggg cgtagttacc ctcacagatt tgcatttccc |
| 2881 | acgctttgag ttcagatggg gggatcatgt ctacctgcgg ggcgatgaag aaaacggttt |
| 2941 | ccggggtagg ggagatcagc tgggaagaaa gcaggttcct gagcagctgc gacttaccgc |
| 3001 | agccggtggg cccgtaaatc acacctatta ccgggtgcaa ctggtagtta agagagctgc |
| 3061 | agctgccgtc atccctgagc aggggggcca cttcgttaag catgtccctg actcgcatgt |
| 3121 | tttccctgac caaatccgcc agaaggcgct cgccgcccag cgatagcagt tcttgcaagg |
| 3181 | aagcaaagtt tttcaacggt ttgagaccgt ccgccgtagg catgcttttg agcgtttgac |
| 3241 | caagcagttc caggcggtcc cacagctcgg tcacctgctc tacggcatct cgatccagca |
| 3301 | tatctcctcg tttcgcgggt tggggcggct ttcgctgtac ggcagtagtc ggtgctcgtc |
| 3361 | cagacgggcc agggtcatgt ctttccacgg gcgcagggtc ctcgtcagcg tagtctgggt |
| 3421 | cacggtgaag gggtgcgctc cgggctgcgc gctggccagg gtgcgcttga ggctggtcct |
| 3481 | gctggtgctg aagcgctgcc ggtcttcgcc ctgcgcgtcg gccaggtagc atttgaccat |
| 3541 | ggtgtcatag tccagcccct ccgcggcgtg gcccttggcg cgcagcttgc ccttggagga |
| 3601 | ggcgccgcac gaggggcagt gcagactttt gagggcgtag agcttgggcg cgagaaatac |
| 3661 | cgattccggg gagtaggcat ccgcgccgca ggccccgcag acggtctcgc attccacgag |
| 3721 | ccaggtgagc tctggccgtt cggggtcaaa aaccaggttt cccccatgct ttttgatgcg |
| 3781 | tttcttacct ctggtttcca tgagccggtg tccacgctcg gtgacgaaaa ggctgtccgt |
| 3841 | gtccccgtat acagacttga gaggcctgtc ctcgagcggt gttccgcggt cctcctcgta |
| 3901 | tagaaactcg gaccactctg agacaaaggc tcgcgtccag gccagcacga aggaggctaa |
| 3961 | gtgggagggg tagcggtcgt tgtccactag ggggtccact cgctccaggg tgtgaagaca |
| 4021 | catgtcgccc tcttcggcat caaggaaggt gattggtttg taggtgtagg ccacgtgacc |
| 4081 | gggtgttcct gaaggggggc tataaaaggg ggtgggggcg cgttcgtcct cactctcttc |
| 4141 | cgcatcgctg tctgcgaggg ccagctgttg gggtgagtac tccctctgaa aagcgggcat |
| 4201 | gacttctgcg ctaagattgt cagtttccaa aaacgaggag gatttgatat tcacctggcc |
| 4261 | cgcggtgatg cctttgaggg tggccgcatc catctggtca gaaaagacaa tctttttgtt |
| 4321 | gtcaagcttg gtggcaaacg acccgtagag ggcgttggac agcaacttgg cgatggagcg |
| 4381 | cagggtttgg tttttgtcgc gatcggcgcg ctccttggcc gcgatgttta gctgcacgta |
| 4441 | ttcgcgcgca acgcaccgcc attcgggaaa gacggtggtg cgctcgtcgg gcaccaggtg |
| 4501 | cacgcgccaa ccgcggttgt gcagggtgac aaggtcaacg ctggtggcta cctctccgcg |
| 4561 | taggcgctcg ttggtccagc agaggcggcc gcccttgcgc gagcagaatg gcggtagggg |
| 4621 | gtctagctgc gtctcgtccg gggggtctgc gtccacggta aagaccccgg gcagcaggcg |
| 4681 | cgcgtcgaag tagtctatct tgcatccttg caagtctagc gcctgctgcc atgcgcgggc |
| 4741 | ggcaagcgcg cgctcgtatg ggttgagtgg gggaccccat ggcatggggt gggtgagcgc |
| 4801 | ggaggcgtac atgccgcaaa tgtcgtaaac gtagaggggc tctctgagta ttccaagata |
| 4861 | tgtagggtag catcttccac cgcggatgct ggcgcgcacg taatcgtata gttcgtgcga |
| 4921 | gggagcgagg aggtcgggac cgaggttgct acgggcgggc tgctctgctc ggaagactat |
| 4981 | ctgcctgaag atggcatgtg agttggatga tatggttgga cgctggaaga cgttgaagct |
| 5041 | ggcgtctgtg agacctaccg cgtcacgcac gaaggaggcg taggagtcgc gcagcttgtt |
| 5101 | gaccagctcg gcggtgacct gcacgtctag ggcgcagtag tccagggttt ccttgatgat |
| 5161 | gtcatactta tcctgtccct tttttttcca cagctcgcgg ttgaggacaa actcttcgcg |
| 5221 | gtctttccag tactcttgga tcggaaaccc gtcggcctcc gaacggtaag agcctagcat |
| 5281 | gtagaactgg ttgacggcct ggtaggcgca gcatcccttt tctacgggta gcgcgtatgc |
| 5341 | ctgcgcggcc ttccggagcg aggtgtgggt gagcgcaaag gtgtccctga ccatgacttt |
| 5401 | gaggtactgg tatttgaagt cagtgtcgtc gcatccgccc tgctcccaga gcaaaaagtc |
| 5461 | cgtgcgcttt ttggaacgcg gatttggcag ggcgaaggtg acatcgttga agagtatctt |
| 5521 | tcccgcgcga ggcataaagt tgcgtgtgat gcggaagggt cccggcacct cggaacggtt |
| 5581 | gttaattacc tgggcggcga gcacgatctc gtcaaagccg ttgatgttgt ggcccacaat |
| 5641 | gtaaagttcc aagaagcgcg ggatgccctt gatggaaggc aattttttaa gttcctcgta |
| 5701 | ggtgagctct tcaggggagc tgagcccgtg ctctgaaagg gcccagtctg caagatgagg |
| 5761 | gttggaagcg acgaatgagc tccacaggtc acgggccatt agcatttgca ggtggtcgcg |
| 5821 | aaaggtccta aactggcgac ctatggccat tttttctggg gtgatgcagt agaaggtaag |
| 5881 | cgggtcttgt tcccagcggt cccatccaag gttcgcggct aggtctcgcg cggcagtcac |
| 5941 | tagaggctca tctccgccga acttcatgac cagcatgaag ggcacgagct gcttcccaaa |
| 6001 | ggcccccatc caagtatagg tctctacatc gtaggtgaca aagagacgct cggtgcgagg |
| 6061 | atgcgagccg atcgggaaga actggatctc ccgccaccaa ttggaggagt ggctattgat |
| 6121 | gtggtgaaag tagaagtccc tgcgacgggc cgaacactcg tgctggcttt tgtaaaaacg |
| 6181 | tgcgcagtac tggcagcggt gcacgggctg tacatcctgc acgaggttga cctgacgacc |
| 6241 | gcgcacaagg aagcagagtg ggaatttgag cccctcgcct ggcgggtttg gctggtggtc |
| 6301 | ttctacttcg gctgcttgtc cttgaccgtc tggctgctcg aggggagtta cggtggatcg |
| 6361 | gaccaccacg ccgcgcgagc ccaaagtcca gatgtccgcg cgcggcggtc ggagcttgat |
| 6421 | gacaacatcg cgcagatggg agctgtccat ggtctggagc tcccgcggcg tcaggtcagg |
| 6481 | cgggagctcc tgcaggttta cctcgcatag acgggtcagg gcgcgggcta gatccaggtg |
| 6541 | atacctaatt tccaggggct ggttggtggc ggcgtcgatg gcttgcaaga ggccgcatcc |
| 6601 | ccgcggcgcg actacggtac cgcgcggcgg gcggtgggcc gcgggggtgt ccttggatga |
| 6661 | tgcatctaaa agcggtgacg cgggcgagcc cccggaggta gggggggctc cggacccgcc |
| 6721 | gggagagggg gcaggggcac gtcggcgccg cgcgcgggca ggagctggtg ctgcgcgcgt |
| 6781 | aggttgctgg cgaacgcgac gacgcggcgg ttgatctcct gaatctggcg cctctgcgtg |
| 6841 | aagacgacgg gcccggtgag cttgagcctg aaagagagtt cgacagaatc aatttcggtg |
| 6901 | tcgttgacgg cggcctggcg caaaatctcc tgcacgtctc ctgagttgtc ttgataggcg |
| 6961 | atctcggcca tgaactgctc gatctcttcc tcctggagat ctccgcgtcc ggctcgctcc |
| 7021 | acggtggcgg cgaggtcgtt ggaaatgcgg gccatgagct gcgagaaggc gttgaggcct |
| 7081 | ccctcgttcc agacgcggct gtagaccacg cccccttcgg catcgcgggc gcgcatgacc |
| 7141 | acctgcgcga gattgagctc cacgtgccgg gcgaagacgg cgtagtttcg caggcgctga |
| 7201 | aagaggtagt tgagggtggt ggcggtgtgt tctgccacga agaagtacat aacccagcgt |
| 7261 | cgcaacgtgg attcgttgat atcccccaag gcctcaaggc gctccatggc ctcgtagaag |
| 7321 | tccacggcga agttgaaaaa ctgggagttg cgcgccgaca cggttaactc ctcctccaga |
| 7381 | agacggatga gctcggcgac agtgtcgcgc acctcgcgct caaaggctac aggggcctct |
| 7441 | tcttcttctt caatctcctc ttccataagg gcctcccctt cttcttcttc tggcggcggt |
| 7501 | gggggagggg ggacacggcg gcgacgacgg cgcaccggga ggcggtcgac aaagcgctcg |
| 7561 | atcatctccc cgcggcgacg gcgcatggtc tcggtgacgg cgcggccgtt ctcgcggggg |
| 7621 | cgcagttgga agacgccgcc cgtcatgtcc cggttatggg ttggcggggg gctgccatgc |
| 7681 | ggcagggata cggcgctaac gatgcatctc aacaattgtt gtgtaggtac tccgccgccg |
| 7741 | agggacctga gcgagtccgc atcgaccgga tcggaaaacc tctcgagaaa ggcgtctaac |
| 7801 | cagtcacagt cgcaaggtag gctgagcacc gtggcgggcg gcagcgggcg gcggtcgggg |
| 7861 | ttgtttctgg cggaggtgct gctgatgatg taattaaagt aggcggtctt gagacggcgg |
| 7921 | atggtcgaca gaagcaccat gtccttgggt ccggcctgct gaatgcgcag gcggtcggcc |
| 7981 | atgccccagg cttcgttttg acatcggcgc aggtctttgt agtagtcttg catgagcctt |
| 8041 | tctaccggca cttcttcttc tccttcctct tgtcctgcat ctcttgcatc tatcgctgcg |
| 8101 | gcggcggcgg agtttggccg taggtggcgc cctcttcctc ccatgcgtgt gaccccgaag |
| 8161 | cccctcatcg gctgaagcag ggctaggtcg gcgacaacgc gctcggctaa tatggcctgc |
| 8221 | tgcacctgcg tgagggtaga ctggaagtca tccatgtcca caaagcggtg gtatgcgccc |
| 8281 | gtgttgatgg tgtaagtgca gttggccata acggaccagt taacggtctg gtgacccggc |
| 8341 | tgcgagagct cggtgtacct gagacgcgag taagccctcg agtcaaatac gtagtcgttg |
| 8401 | caagtccgca ccaggtactg gtatcccacc aaaaagtgcg gcggcggctg gcggtagagg |
| 8461 | ggccagcgta gggtggccgg ggctccgggg gcgagatctt ccaacataag gcgatgatat |
| 8521 | ccgtagatgt acctggacat ccaggtgatg ccggcggcgg tggtggaggc gcgcggaaag |
| 8581 | tcgcggacgc ggttccagat gttgcgcagc ggcaaaaagt gctccatggt cgggacgctc |
| 8641 | tggccggtca ggcgcgcgca atcgttgacg ctctagaccg tgcaaaagga gagcctgtaa |
| 8701 | gcgggcactc ttccgtggtc tggtggataa attcgcaagg gtatcatggc ggacgaccgg |
| 8761 | ggttcgagcc ccgtatccgg ccgtccgccg tgatccatgc ggttaccgcc cgcgtgtcga |
| 8821 | acccaggtgt gcgacgtcag acaacggggg agtgctcctt ttggcttcct tccaggcgcg |
| 8881 | gcggctgctg cgctagcttt tttggccact ggccgcgcgc agcgtaagcg gttaggctgg |
| 8941 | aaagcgaaag cattaagtgg ctcgctccct gtagccggag ggttattttc caagggttga |
| 9001 | gtcgcgggac ccccggttcg agtctcggac cggccggact gcggcgaacg ggggtttgcc |
| 9061 | tccccgtcat gcaagacccc gcttgcaaat tcctccggaa acagggacga gccccttttt |
| 9121 | tgcttttccc agatgcatcc ggtgctgcgg cagatgcgcc cccctcctca gcagcggcaa |
| 9181 | gagcaagagc agcggcagac atgcagggca ccctcccctc ctcctaccgc gtcaggaggg |
| 9241 | gcgacatccg cggttgacgc ggcagcagat ggtgattacg aacccccgcg gcgccgggcc |
| 9301 | cggcactacc tggacttgga ggagggcgag ggcctggcgc ggctaggagc gccctctcct |
| 9361 | gagcggtacc caagggtgca gctgaagcgt gatacgcgtg aggcgtacgt gccgcggcag |
| 9421 | aacctgtttc gcgaccgcga gggagaggag cccgaggaga tgcgggatcg aaagttccac |
| 9481 | gcagggcgcg agctgcggca tggcctgaat cgcgagcggt tgctgcgcga ggaggacttt |
| 9541 | gagcccgacg cgcgaaccgg gattagtccc gcgcgcgcac acgtggcggc cgccgacctg |
| 9601 | gtaaccgcat acgagcagac ggtgaaccag gagattaact ttcaaaaaag ctttaacaac |
| 9661 | cacgtgcgta cgcttgtggc gcgcgaggag gtggctatag gactgatgca tctgtgggac |
| 9721 | tttgtaagcg cgctggagca aaacccaaat agcaagccgc tcatggcgca gctgttcctt |
| 9781 | atagtgcagc acagcaggga caacgaggca ttcagggatg cgctgctaaa catagtagag |
| 9841 | cccgagggcc gctggctgct cgatttgata aacatcctgc agagcatagt ggtgcaggag |
| 9901 | cgcagcttga gcctggctga caaggtggcc gccatcaact attccatgct tagcctgggc |
| 9961 | aagttttacg cccgcaagat ataccatacc ccttacgttc ccatagacaa ggaggtaaag |
| 10021 | atcgaggggt tctacatgcg catggcgctg aaggtgctta ccttgagcga cgacctgggc |
| 10081 | gtttatcgca acgagcgcat ccacaaggcc gtgagcgtga gccggcggcg cgagctcagc |
| 10141 | gaccgcgagc tgatgcacag cctgcaaagg gccctggctg gcacgggcag cggcgataga |
| 10201 | gaggccgagt cctactttga cgcgggcgct gacctgcgct gggccccaag ccgacgcgcc |
| 10261 | ctggaggcag ctggggccgg acctgggctg gcggtggcac ccgcgcgcgc tggcaacgtc |
| 10321 | ggcggcgtgg aggaatatga cgaggacgat gagtacgagc cagaggacgg cgagtactaa |
| 10381 | gcggtgatgt ttctgatcag atgatgcaag acgcaacgga cccggcggtg cgggcggcgc |
| 10441 | tgcagagcca gccgtccggc cttaactcca cggacgactg gcgccaggtc atggaccgca |
| 10501 | tcatgtcgct gactgcgcgc aatcctgacg cgttccggca gcagccgcag gccaaccggc |
| 10561 | tctccgcaat tctggaagcg gtggtcccgg cgcgcgcaaa ccccacgcac gagaaggtgc |
| 10621 | tggcgatcgt aaacgcgctg gccgaaaaca gggccatccg gcccgacgag gccggcctgg |
| 10681 | tctacgacgc gctgcttcag cgcgtggctc gttacaacag cggcaacgtg cagaccaacc |
| 10741 | tggaccggct ggtgggggat gtgcgcgagg ccgtggcgca gcgtgagcgc gcgcagcagc |
| 10801 | agggcaacct gggctccatg gttgcactaa acgccttcct gagtacacag cccgccaacg |
| 10861 | tgccgcgggg acaggaggac tacaccaact ttgtgagcgc actgcggcta atggtgactg |
| 10921 | agacaccgca aagtgaggtg taccagtctg ggccagacta ttttttccag accagtagac |
| 10981 | aaggcctgca gaccgtaaac ctgagccagg ctttcaaaaa cttgcagggg ctgtgggggg |
| 11041 | tgcgggctcc cacaggcgac cgcgcgaccg tgtctagctt gctgacgccc aactcgcgcc |
| 11101 | tgttgctgct gctaatagcg cccttcacgg acagtggcag cgtgtcccgg gacacatacc |
| 11161 | taggtcactt gctgacactg taccgcgagg ccataggtca ggcgcatgtg gacgagcata |
| 11221 | ctttccagga gattacaagt gtcagccgcg cgctggggca ggaggacacg ggcagcctgg |
| 11281 | aggcaaccct aaactacctg ctgaccaacc ggcggcagaa gatcccctcg ttgcacagtt |
| 11341 | taaacagcga ggaggagcgc attttgcgct acgtgcagca gagcgtgagc cttaacctga |
| 11401 | tgcgcgacgg ggtaacgccc agcgtggcgc tggacatgac cgcgcgcaac atggaaccgg |
| 11461 | gcatgtatgc ctcaaaccgg ccgtttatca accgcctaat ggactacttg catcgcgcgg |
| 11521 | ccgccgtgaa ccccgagtat ttcaccaatg ccatcttgaa cccgcactgg ctaccgcccc |
| 11581 | ctggtttcta caccggggga ttcgaggtgc ccgagggtaa cgatggattc ctctgggacg |
| 11641 | acatagacga cagcgtgttt tccccgcaac cgcagaccct gctagagttg caacagcgcg |
| 11701 | agcaggcaga ggcggcgctg cgaaaggaaa gcttccgcag gccaagcagc ttgtccgatc |
| 11761 | taggcgctgc ggccccgcgg tcagatgcta gtagcccatt tccaagcttg atagggtctc |
| 11821 | ttaccagcac tcgcaccacc cgcccgcgcc tgctgggcga ggaggagtac ctaaacaact |
| 11881 | cgctgctgca gccgcagcgc gaaaaaaacc tgcctccggc atttcccaac aacgggatag |
| 11941 | agagcctagt ggacaagatg agtagatgga agacgtacgc gcaggagcac agggacgtgc |
| 12001 | caggcccgcg cccgcccacc cgtcgtcaaa ggcacgaccg tcagcggggt ctggtgtggg |
| 12061 | aggacgatga ctcggcagac gacagcagcg tcctggattt gggagggagt ggcaacccgt |
| 12121 | ttgcgcacct tcgccccagg ctggggagaa tgttttaaaa aaaaaaaagc atgatgcaaa |
| 12181 | ataaaaaact caccaaggcc atggcaccga gcgttggttt tcttgtattc cccttagtat |
| 12241 | gcggcgcgcg gcgatgtatg aggaaggtcc tcctccctcc tacgagagtg tggtgagcgc |
| 12301 | ggcgccagtg gcggcggcgc tgggttctcc cttcgatgct cccctggacc cgccgtttgt |
| 12361 | gcctccgcgg tacctgcggc ctaccggggg gagaaacagc atccgttact ctgagttggc |
| 12421 | acccctattc gacaccaccc gtgtgtacct ggtggacaac aagtcaacgg atgtggcatc |
| 12481 | cctgaactac cagaacgacc acagcaactt tctgaccacg gtcattcaaa acaatgacta |
| 12541 | cagcccgggg gaggcaagca cacagaccat caatcttgac gaccggtcgc actggggcgg |
| 12601 | cgacctgaaa accatcctgc ataccaacat gccaaatgtg aacgagttca tgtttaccaa |
| 12661 | taagtttaag gcgcgggtga tggtgtcgcg cttgcctact aaggacaatc aggtggagct |
| 12721 | gaaatacgag tgggtggagt tcacgctgcc cgagggcaac tactccgaga ccatgaccat |
| 12781 | agaccttatg aacaacgcga tcgtggagca ctacttgaaa gtgggcagac agaacggggt |
| 12841 | tctggaaagc gacatcgggg taaagtttga cacccgcaac ttcagactgg ggtttgaccc |
| 12901 | cgtcactggt cttgtcatgc ctggggtata tacaaacgaa gccttccatc cagacatcat |
| 12961 | tttgctgcca ggatgcgggg tggacttcac ccacagccgc ctgagcaact tgttgggcat |
| 13021 | ccgcaagcgg caacccttcc aggagggctt taggatcacc tacgatgatc tggagggtgg |
| 13081 | taacattccc gcactgttgg atgtggacgc ctaccaggcg agcttgaaag atgacaccga |
| 13141 | acagggcggg ggtggcgcag gcggcagcaa cagcagtggc agcggcgcgg aagagaactc |
| 13201 | caacgcggca gccgcggcaa tgcagccggt ggaggacatg aacgatcatg ccattcgcgg |
| 13261 | cgacaccttt gccacacggg ctgaggagaa gcgcgctgag gccgaagcag cggccgaagc |
| 13321 | tgccgccccc gctgcgcaac ccgaggtcga gaagcctcag aagaaaccgg tgatcaaacc |
| 13381 | cctgacagag gacagcaaga aacgcagtta caacctaata agcaatgaca gcaccttcac |
| 13441 | ccagtaccgc agctggtacc ttgcatacaa ctacggcgac cctcagaccg gaatccgctc |
| 13501 | atggaccctg ctttgcactc ctgacgtaac ctgcggctcg gagcaggtct actggtcgtt |
| 13561 | gccagacatg atgcaagacc ccgtgacctt ccgctccacg cgccagatca gcaactttcc |
| 13621 | ggtggtgggc gccgagctgt tgcccgtgca ctccaagagc ttctacaacg accaggccgt |
| 13681 | ctactcccaa ctcatccgcc agtttacctc tctgacccac gtgttcaatc gctttcccga |
| 13741 | gaaccagatt ttggcgcgcc cgccagcccc caccatcacc accgtcagtg aaaacgttcc |
| 13801 | tgctctcaca gatcacggga cgctaccgct gcgcaacagc atcggaggag tccagcgagt |
| 13861 | gaccattact gacgccagac gccgcacctg cccctacgtt tacaaggccc tgggcatagt |
| 13921 | ctcgccgcgc gtcctatcga gccgcacttt ttgagcaagc atgtccatcc ttatatcgcc |
| 13981 | cagcaataac acaggctggg gcctgcgctt cccaagcaag atgtttggcg gggccaagaa |
| 14041 | gcgctccgac caacacccag tgcgcgtgcg cgggcactac cgcgcgccct ggggcgcgca |
| 14101 | caaacgcggc cgcactgggc gcaccaccgt cgatgacgcc atcgacgcgg tggtggagga |
| 14161 | ggcgcgcaac tacacgccca cgccgccacc agtgtccaca gtggacgcgg ccattcagac |
| 14221 | cgtggtgcgc ggagcccggc gctatgctaa aatgaagaga cggcggaggc gcgtagcacg |
| 14281 | tcgccaccgc cgccgacccg gcactgccgc ccaacgcgcg gcggcggccc tgcttaaccg |
| 14341 | cgcacgtcgc accggccgac gggcggccat gcgggccgct cgaaggctgg ccgcgggtat |
| 14401 | tgtcactgtg ccccccaggt ccaggcgacg agcggccgcc gcagcagccg cggccattag |
| 14461 | tgctatgact cagggtcgca ggggcaacgt gtattgggtg cgcgactcgg ttagcggcct |
| 14521 | gcgcgtgccc gtgcgcaccc gccccccgcg caactagatt gcaagaaaaa actacttaga |
| 14581 | ctcgtactgt tgtatgtatc cagcggcggc ggcgcgcaac gaagctatgt ccaagcgcaa |
| 14641 | aatcaaagaa gagatgctcc aggtcatcgc gccggagatc tatggccccc cgaagaagga |
| 14701 | agagcaggat tacaagcccc gaaagctaaa gcgggtcaaa aagaaaaaga aagatgatga |
| 14761 | tgatgaactt gacgacgagg tggaactgct gcacgctacc gcgcccaggc gacgggtaca |
| 14821 | gtggaaaggt cgacgcgtaa aacgtgtttt gcgacccggc accaccgtag tctttacgcc |
| 14881 | cggtgagcgc tccacccgca cctacaagcg cgtgtatgat gaggtgtacg gcgacgagga |
| 14941 | cctgcttgag caggccaacg agcgcctcgg ggagtttgcc tacggaaagc ggcataagga |
| 15001 | catgctggcg ttgccgctgg acgagggcaa cccaacacct agcctaaagc ccgtaacact |
| 15061 | gcagcaggtg ctgcccgcgc ttgcaccgtc cgaagaaaag cgcggcctaa agcgcgagtc |
| 15121 | tggtgacttg gcacccaccg tgcagctgat ggtacccaag cgccagcgac tggaagatgt |
| 15181 | cttggaaaaa atgaccgtgg aacctgggct ggagcccgag gtccgcgtgc ggccaatcaa |
| 15241 | gcaggtggcg ccgggactgg gcgtgcagac cgtggacgtt cagataccca ctaccagtag |
| 15301 | caccagtatt gccaccgcca cagagggcat ggagacacaa acgtccccgg ttgcctcagc |
| 15361 | ggtggcggat gccgcggtgc aggcggtcgc tgcggccgcg tccaagacct ctacggaggt |
| 15421 | gcaaacggac ccgtggatgt ttcgcgtttc agccccccgg cgcccgcgcg gttcgaggaa |
| 15481 | gtacggcgcc gccagcgcgc tactgcccga atatgcccta catccttcca ttgcgcctac |
| 15541 | ccccggctat cgtggctaca cctaccgccc cagaagacga gcaactaccc gacgccgaac |
| 15601 | caccactgga acccgccgcc gccgtcgccg tcgccagccc gtgctggccc cgatttccgt |
| 15661 | gcgcagggtg gctcgcgaag gaggcaggac cctggtgctg ccaacagcgc gctaccaccc |
| 15721 | cagcatcgtt taaaagccgg tctttgtggt tcttgcagat atggccctca cctgccgcct |
| 15781 | ccgtttcccg gtgccgggat tccgaggaag aatgcaccgt aggaggggca tggccggcca |
| 15841 | cggcctgacg ggcggcatgc gtcgtgcgcac caccggcgg cggcgcgcgt cgcaccgtcg |
| 15901 | catgcgcggc ggtatcctgc ccctccttat tccactgatc gccgcggcga ttggcgccgt |
| 15961 | gcccggaatt gcatccgtgg ccttgcaggc gcagagacac tgattaaaaa caagttgcat |
| 16021 | gtggaaaaat caaaataaaa agtctggact ctcacgctcg cttggtcctg taactatttt |
| 16081 | gtagaatgga agacatcaac tttgcgtctc tggccccgcg acacggctcg cgcccgttca |
| 16141 | tgggaaactg gcaagatatc ggcaccagca atatgagcgg tggcgccttc agctggggct |
| 16201 | cgctgtggag cggcattaaa aatttcggtt ccaccgttaa gaactatggc agcaaggcct |
| 16261 | ggaacagcag cacaggccag atgctgaggg ataagttgaa agagcaaaat ttccaacaaa |
| 16321 | aggtggtaga tggcctggcc tctggcatta gcggggtggt ggacctggcc aaccaggcag |
| 16381 | tgcaaaataa gattaacagt aagcttgatc cccgccctcc cgtagaggag cctccaccgg |
| 16441 | ccgtggagac agtgtctcca gaggggcgtg gcgaaaagcg tccgcgcccc gacagggaag |
| 16501 | aaactctggt gacgcaaata gacgagcctc cctcgtacga ggaggcacta aagcaaggcc |
| 16561 | tgcccaccac ccgtcccatc gcgcccatgg ctaccggagt gctgggccag cacacacccg |
| 16621 | taacgctgga cctgcctccc cccgccgaca cccagcagaa acctgtgctg ccaggcccga |
| 16681 | ccgccgttgt tgtaacccgt cctagccgcg cgtccctgcg ccgcgccgcc agcggtccgc |
| 16741 | gatcgttgcg gcccgtagcc agtggcaact ggcaaagcac actgaacagc atcgtgggtc |
| 16801 | tgggggtgca atccctgaag cgccgacgat gcttctgaat agctaacgtg tcgtatgtgt |
| 16861 | gtcatgtatg cgtccatgtc gccgccagag gagctgctga gccgccgcgc gcccgctttc |
| 16921 | caagatggct accccttcga tgatgccgca gtggtcttac atgcacatct cgggccagga |
| 16981 | cgcctcggag tacctgagcc ccgggctggt gcagtttgcc cgcgccaccg agacgtactt |
| 17041 | cagcctgaat aacaagttta gaaaccccac ggtggcgcct acgcacgacg tgaccacaga |
| 17101 | ccggtcccag cgtttgacgc tgcggttcat ccctgtggac cgtgaggata ctgcgtactc |
| 17161 | gtacaaggcg cggttcaccc tagctgtggg tgataaccgt gtgctggaca tggcttccac |
| 17221 | gtactttgac atccgcggcg tgctggacag gggccctact tttaagccct actctggcac |
| 17281 | tgcctacaac gccctggctc ccaagggtgc cccaaatcct tgcgaatggg atgaagctgc |
| 17341 | tactgctctt gaaataaacc tagaagaaga ggacgatgac aacgaagacg aagtagacga |
| 17401 | gcaagctgag cagcaaaaaa ctcacgtatt tgggcaggcg ccttattctg gtataaatat |
| 17461 | tacaaaggag ggtattcaaa taggtgtcga aggtcaaaca cctaaatatg ccgataaaac |
| 17521 | atttcaacct gaacctcaaa taggagaatc tcagtggtac gaaactgaaa ttaatcatgc |
| 17581 | agctgggaga gtccttaaaa agactacccc aatgaaacca tgttacggtt catatgcaaa |
| 17641 | acccacaaat gaaaatggag ggcaaggcat tcttgtaaag caacaaaatg gaaagctaga |
| 17701 | aagtcaagtg gaaatgcaat ttttctcaac tactgaggcg accgcaggca atggtgataa |
| 17761 | cttgactcct aaagtggtat tgtacagtga agatgtagat atagaaaccc cagacactca |
| 17821 | tatttcttac atgcccacta ttaaggaagg taactcacga gaactaatgg gccaacaatc |
| 17881 | tatgcccaac aggcctaatt acattgcttt tagggacaat tttattggtc taatgtatta |
| 17941 | caacagcacg ggtaatatgg gtgttctggc gggccaagca tcgcagttga atgctgttgt |
| 18001 | agatttgcaa gacagaaaca cagagctttc ataccagctt ttgcttgatt ccattggtga |
| 18061 | tagaaccagg tacttttcta tgtggaatca ggctgttgac agctatgatc cagatgttag |
| 18121 | aattattgaa aatcatggaa ctgaagatga acttccaaat tactgctttc cactgggagg |
| 18181 | tgtgattaat acagagactc ttaccaaggt aaaacctaaa acaggtcagg aaaatggatg |
| 18241 | ggaaaaagat gctacagaat tttcagataa aaatgaaata agagttggaa ataattttgc |
| 18301 | catggaaatc aatctaaatg ccaacctgtg gagaaatttc ctgtactcca acatagcgct |
| 18361 | gtatttgccc gacaagctaa agtacagtcc ttccaacgta aaaatttctg ataacccaaa |
| 18421 | cacctacgac tacatgaaca agcgagtggt ggctcccggg ttagtggact gctacattaa |
| 18481 | ccttggagca cgctggtccc ttgactatat ggacaacgtc aacccattta accaccaccg |
| 18541 | caatgctggc ctgcgctacc gctcaatgtt gctgggcaat ggtcgctatg tgcccttcca |
| 18601 | catccaggtg cctcagaagt tctttgccat taaaaacctc cttctcctgc cgggctcata |
| 18661 | cacctacgag tggaacttca ggaaggatgt taacatggtt ctgcagagct ccctaggaaa |
| 18721 | tgacctaagg gttgacggag ccagcattaa gtttgatagc atttgccttt acgccacctt |
| 18781 | cttccccatg gcccacaaca ccgcctccac gcttgaggcc atgcttagaa acgacaccaa |
| 18841 | cgaccagtcc tttaacgact atctctccgc cgccaacatg ctctacccta tacccgccaa |
| 18901 | cgctaccaac gtgcccatat ccatcccctc ccgcaactgg gcggctttcc gcggctgggc |
| 18961 | cttcacgcgc cttaagacta aggaaacccc atcactgggc tcgggctacg acccttatta |
| 19021 | cacctactct ggctctatac cctacctaga tggaaccttt tacctcaacc acacctttaa |
| 19081 | gaaggtggcc attacctttg actcttctgt cagctggcct ggcaatgacc gcctgcttac |
| 19141 | ccccaacgag tttgaaatta agcgctcagt tgacggggag ggttacaacg ttgcccagtg |
| 19201 | taacatgacc aaagactggt tcctggtaca aatgctagct aactacaaca ttggctacca |
| 19261 | gggcttctat atcccagaga gctacaagga ccgcatgtac tccttcttta gaaacttcca |
| 19321 | gcccatgagc cgtcaggtgg tggatgatac taaatacaag gactaccaac aggtgggcat |
| 19381 | cctacaccaa cacaacaact ctggatttgt tggctacctt gcccccacca tgcgcgaagg |
| 19441 | acaggcctac cctgctaact tcccctatcc gcttataggc aagaccgcag ttgacagcat |
| 19501 | tacccagaaa aagtttcttt gcgatcgcac cctttggcgc atcccattct ccagtaactt |
| 19561 | tatgtccatg ggcgcactca cagacctggg ccaaaacctt ctctacgcca actccgccca |
| 19621 | cgcgctagac atgacttttg aggtggatcc catggacgag cccacccttc tttatgtttt |
| 19681 | gtttgaagtc tttgacgtgg tccgtgtgca ccggccgcac cgcggcgtca tcgaaaccgt |
| 19741 | gtacctgcgc acgcccttct cggccggcaa cgccacaaca taaagaagca agcaacatca |
| 19801 | acaacagctg ccgccatggg ctccagtgag caggaactga aagccattgt caaagatctt |
| 19861 | ggttgtgggc catatttttt gggcacctat gacaagcgct ttccaggctt tgtttctcca |
| 19921 | cacaagctcg cctgcgccat agtcaatacg gccggtcgcg agactggggg cgtacactgg |
| 19981 | atggcctttg cctggaaccc gcactcaaaa acatgctacc tctttgagcc ctttggcttt |
| 20041 | tctgaccagc gactcaagca ggtttaccag tttgagtacg agtcactcct gcgccgtagc |
| 20101 | gccattgctt cttcccccga ccgctgtata acgctggaaa agtccaccca aagcgtacag |
| 20161 | gggcccaact cggccgcctg tggactattc tgctgcatgt ttctccacgc ctttgccaac |
| 20221 | tggccccaaa ctcccatgga tcacaacccc accatgaacc ttattaccgg ggtacccaac |
| 20281 | tccatgctca acagtcccca ggtacagccc accctgcgtc gcaaccagga acagctctac |
| 20341 | agcttcctgg agcgccactc gccctacttc cgcagccaca gtgcgcagat taggagcgcc |
| 20401 | acttcttttt gtcacttgaa aaacatgtaa aaataatgta ctagagacac tttcaataaa |
| 20461 | ggcaaatgct tttatttgta cactctcggg tgattattta cccccaccct tgccgtctgc |
| 20521 | gccgtttaaa aatcaaaggg gttctgccgc gcatcgctat gcgccactgg cagggacacg |
| 20581 | ttgcgatact ggtgtttagt gctccactta aactcaggca caaccatccg cggcagctcg |
| 20641 | gtgaagtttt cactccacag gctgcgcacc atcaccaacg cgtttagcag gtcgggcgcc |
| 20701 | gatatcttga agtcgcagtt ggggcctccg ccctgcgcgc gcgagttgcg atacacaggg |
| 20761 | ttgcagcact ggaacactat cagcgccggg tggtgcacgc tggccagcac gctcttgtcg |
| 20821 | gagatcagat ccgcgtccag gtcctccgcg ttgctcaggg cgaacggagt caactttggt |
| 20881 | agctgccttc ccaaaaaggg cgcgtgccca ggctttgagt tgcactcgca ccgtagtggc |
| 20941 | atcaaaaggt gaccgtgccc ggtctgggcg ttaggataca gcgcctgcat aaaagccttg |
| 21001 | atctgcttaa aagccacctg agcctttgcg ccttcagaga agaacatgcc gcaagacttg |
| 21061 | ccggaaaact gattggccgg acaggccgcg tcgtgcacgc agcaccttgc gtcggtgttg |
| 21121 | gagatctgca ccacatttcg gccccaccgg ttcttcacga tcttggcctt gctagactgc |
| 21181 | tccttcagcg cgcgctgccc gttttcgctc gtcacatcca tttcaatcac gtgctcctta |
| 21241 | tttatcataa tgcttccgtg tagacactta agctcgcctt cgatctcagc gcagcggtgc |
| 21301 | agccacaacg cgcagcccgt gggctcgtga tgcttgtagg tcacctctgc aaacgactgc |
| 21361 | aggtacgcct gcaggaatcg ccccatcatc gtcacaaagg tcttgttgct ggtgaaggtc |
| 21421 | agctgcaacc cgcggtgctc ctcgttcagc caggtcttgc atacggccgc cagagcttcc |
| 21481 | acttggtcag gcagtagttt gaagttcgcc tttagatcgt tatccacgtg gtacttgtcc |
| 21541 | atcagcgcgc gcgcagcctc catgcccttc tcccacgcag acacgatcgg cacactcagc |
| 21601 | gggttcatca ccgtaatttc actttccgct tcgctgggct cttcctcttc ctcttgcgtc |
| 21661 | cgcataccac gcgccactgg gtcgtcttca ttcagccgcc gcactgtgcg cttacctcct |
| 21721 | ttgccatgct tgattagcac cggtgggttg ctgaaaccca ccatttgtag cgccacatct |
| 21781 | tctctttctt cctcgctgtc cacgattacc tctggtgatg gcgggcgctc gggcttggga |
| 21841 | gaagggcgct tctttttctt cttgggcgca atggccaaat ccgccgccga ggtcgatggc |
| 21901 | cgcgggctgg gtgtgcgcgg caccagcgcg tcttgtgatg agtcttcctc gtcctcggac |
| 21961 | tcgatacgcc gcctcatccg cttttttggg ggcgcccggg gaggcggcgg cgacggggac |
| 22021 | ggggacgaca cgtcctccat ggttggggga cgtcgcgccg caccgcgtcc gcgctcgggg |
| 22081 | gtggtttcgc gctgctcctc ttcccgactg gccatttcct tctcctatag gcagaaaaag |
| 22141 | atcatggagt cagtcgagaa gaaggacagc ctaaccgccc cctctgagtt cgccaccacc |
| 22201 | gcctccaccg atgccgccaa cgcgcctacc accttccccg tcgaggcacc cccgcttgag |
| 22261 | gaggaggaag tgattatcga gcaggaccca ggttttgtaa gcgaagacga cgaggaccgc |
| 22321 | tcagtaccaa cagaggataa aaagcaagac caggacaacg cagaggcaaa cgaggaacaa |
| 22381 | gtcgggcggg gggacgaaag gcatggcgac tacctagatg tgggagacga cgtgctgttg |
| 22441 | aagcatctgc agcgccagtg cgccattatc tgcgacgcgt tgcaagagcg cagcgatgtg |
| 22501 | cccctcgcca tagcggatgt cagccttgcc tacgaacgcc acctattctc accgcgcgta |
| 22561 | ccccccaaac gccaagaaaa cggcacatgc gagcccaacc cgcgcctcaa cttctacccc |
| 22621 | gtatttgccg tgccagaggt gcttgccacc tatcacatct ttttccaaaa ctgcaagata |
| 22681 | cccctatcct gccgtgccaa ccgcagccga gcggacaagc agctggcctt gcggcagggc |
| 22741 | gctgtcatac ctgatatcgc ctcgctcaac gaagtgccaa aaatctttga gggtcttgga |
| 22801 | cgcgacgaga agcgcgcggc aaacgctctg caacaggaaa acagcgaaaa tgaaagtcac |
| 22861 | tctggagtgt tggtggaact cgagggtgac aacgcgcgcc tagccgtact aaaacgcagc |
| 22921 | atcgaggtca cccactttgc ctacccggca cttaacctac cccccaaggt catgagcaca |
| 22981 | gtcatgagtg agctgatcgt gcgccgtgcg cagcccctgg agagggatgc aaatttgcaa |
| 23041 | gaacaaacag aggagggcct acccgcagtt ggcgacgagc agctagcgcg ctggcttcaa |
| 23101 | acgcgcgagc ctgccgactt ggaggagcga cgcaaactaa tgatggccgc agtgctcgtt |
| 23161 | accgtggagc ttgagtgcat gcagcggttc tttgctgacc cggagatgca gcgcaagcta |
| 23221 | gaggaaacat tgcactacac ctttcgacag ggctacgtac gccaggcctg caagatctcc |
| 23281 | aacgtggagc tctgcaacct ggtctcctac cttggaattt tgcacgaaaa ccgccttggg |
| 23341 | caaaacgtgc ttcattccac gctcaagggc gaggcgcgcc gcgactacgt ccgcgactgc |
| 23401 | gtttacttat ttctatgcta cacctggcag acggccatgg gcgtttggca gcagtgcttg |
| 23461 | gaggagtgca acctcaagga gctgcagaaa ctgctaaagc aaaacttgaa ggacctatgg |
| 23521 | acggccttca acgagcgctc cgtggccgcg cacctggcgg acatcatttt ccccgaacgc |
| 23581 | ctgcttaaaa ccctgcaaca gggtctgcca gacttcacca gtcaaagcat gttgcagaac |
| 23641 | tttaggaact ttatcctaga gcgctcagga atcttgcccg ccacctgctg tgcacttcct |
| 23701 | agcgactttg tgcccattaa gtaccgcgaa tgccctccgc cgctttgggg ccactgctac |
| 23761 | cttctgcagc tagccaacta ccttgcctac cactctgaca taatggaaga cgtgagcggt |
| 23821 | gacggtctac tggagtgtca ctgtcgctgc aacctatgca ccccgcaccg ctccctggtt |
| 23881 | tgcaattcgc agctgcttaa cgaaagtcaa attatcggta cctttgagct gcagggtccc |
| 23941 | tcgcctgacg aaaagtccgc ggctccgggg ttgaaactca ctccggggct gtggacgtcg |
| 24001 | gcttaccttc gcaaatttgt acctgaggac taccacgccc acgagattag gttctacgaa |
| 24061 | gaccaatccc gcccgccaaa tgcggagctt accgcctgcg tcattaccca gggccacatt |
| 24121 | cttggccaat tgcaagccat caacaaagcc cgccaagagt ttctgctacg aaagggacgg |
| 24181 | ggggtttact tggaccccca gtccggcgag gagctcaacc caatcccccc gccgccgcag |
| 24241 | ccctatcagc agcagccgcg ggcccttgct tcccaggatg gcacccaaaa agaagctgca |
| 24301 | gctgccgccg ccacccacgg acgaggagga atactgggac agtcaggcag aggaggtttt |
| 24361 | ggacgaggag gaggaggaca tgatggaaga ctgggagagc ctagacgagg aagcttccga |
| 24421 | ggtcgaagag gtgtcagacg aaacaccgtc accctcggtc gcattcccct cgccggcgcc |
| 24481 | ccagaaatcg gcaaccggtt ccagcatggc tacaacctcc gctcctcagg cgccgccggc |
| 24541 | actgcccgtt cgccgaccca accgtagatg ggacaccact ggaaccaggg ccggtaagtc |
| 24601 | caagcagccg ccgccgttag cccaagagca acaacagcgc caaggctacc gctcatggcg |
| 24661 | cgggcacaag aacgccatag ttgcttgctt gcaagactgt gggggcaaca tctccttcgc |
| 24721 | ccgccgcttt cttctctacc atcacggcgt ggccttcccc cgtaacatcc tgcattacta |
| 24781 | ccgtcatctc tacagcccat actgcaccgg cggcagcggc agcggcagca acagcagcgg |
| 24841 | ccacacagaa gcaaaggcga ccggatagca agactctgac aaagcccaag aaatccacag |
| 24901 | cggcggcagc agcaggagga ggagcgctgc gtctggcgcc caacgaaccc gtatcgaccc |
| 24961 | gcgagcttag aaacaggatt tttcccactc tgtatgctat atttcaacag agcaggggcc |
| 25021 | aagaacaaga gctgaaaata aaaaacaggt ctctgcgatc cctcacccgc agctgcctgt |
| 25081 | atcacaaaag cgaagatcag cttcggcgca cgctggaaga cgcggaggct ctcttcagta |
| 25141 | aatactgcgc gctgactctt aaggactagt ttcgcgccct ttctcaaatt taagcgcgaa |
| 25201 | aactacgtca tctccagcgg ccacacccgg cgccagcacc tgtcgtcagc gccattatga |
| 25261 | gcaaggaaat tcccacgccc tacatgtgga gttaccagcc acaaatggga cttgcggctg |
| 25321 | gagctgccca agactactca acccgaataa actacatgag cgcgggaccc cacatgatat |
| 25381 | cccgggtcaa cggaatccgc gcccaccgaa accgaattct cttggaacag gcggctatta |
| 25441 | ccaccacacc tcgtaataac cttaatcccc gtagttggcc cgctgccctg gtgtaccagg |
| 25501 | aaagtcccgc tcccaccact gtggtacttc ccagagacgc ccaggccgaa gttcagatga |
| 25561 | ctaactcagg ggcgcagctt gcgggcggct ttcgtcacag ggtgcggtcg cccgggcagg |
| 25621 | gtataactca cctgacaatc agagggcgag gtattcagct caacgacgag tcggtgagct |
| 25681 | cctcgcttgg tctccgtccg gacgggacat ttcagatcgg cggcgccggc cgtccttcat |
| 25741 | tcacgcctcg tcaggcaatc ctaactctgc agacctcgtc ctctgagccg cgctctggag |
| 25801 | gcattggaac tctgcaattt attgaggagt ttgtgccatc ggtctacttt aaccccttct |
| 25861 | cgggacctcc cggccactat ccggatcaat ttattcctaa ctttgacgcg gtaaaggact |
| 25921 | cggcggacgg ctacgactga atgttaagtg gagaggcaga gcaactgcgc ctgaaacacc |
| 25981 | tggtccactg tcgccgccac aagtgctttg cccgcgactc cggtgagttt tgctactttg |
| 26041 | aattgcccga ggatcatatc gagggcccgg cgcacggcgt ccggcttacc gcccagggag |
| 26101 | agcttgcccg tagcctgatt cgggagttta cccagcgccc cctgctagtt gagcgggaca |
| 26161 | ggggaccctg tgttctcact gtgatttgca actgtcctaa ccttggatta catcaagatc |
| 26221 | tttgttgcca tctctgtgct gagtataata aatacagaaa ttaaaatata ctggggctcc |
| 26281 | tatcgccatc ctgtaaacgc caccgtcttc acccgcccaa gcaaaccaag gcgaacctta |
| 26341 | cctggtactt ttaacatctc tccctctgtg atttacaaca gtttcaaccc agacggagtg |
| 26401 | agtctacgag agaacctctc cgagctcagc tactccatca gaaaaaacac caccctcctt |
| 26461 | acctgccggg aacgtacgag tgcgtcaccg gccgctgcac cacacctacc gcctgaccgt |
| 26521 | aaaccagact ttttccggac agacctcaat aactctgttt accagaacag gaggtgagct |
| 26581 | tagaaaaccc ttagggtatt aggccaaagg cgcagctact gtggggttta tgaacaattc |
| 26641 | aagcaactct acgggctatt ctaattcagg tttctctaga aatggacgga attattacag |
| 26701 | agcagcgcct gctagaaaga cgcagggcag cggccgagca acagcgcatg aatcaagagc |
| 26761 | tccaagacat ggttaacttg caccagtgca aaaggggtat cttttgtctg gtaaagcagg |
| 26821 | ccaaagtcac ctacgacagt aataccaccg gacaccgcct tagctacaag ttgccaacca |
| 26881 | agcgtcagaa attggtggtc atggtgggag aaaagcccat taccataact cagcactcgg |
| 26941 | tagaaaccga aggctgcatt cactcacctt gtcaaggacc tgaggatctc tgcaccctta |
| 27001 | ttaagaccct gtgcggtctc aaagatctta ttccctttaa ctaataaaaa aaaataataa |
| 27061 | agcatcactt acttaaaatc agttagcaaa tttctgtcca gtttattcag cagcacctcc |
| 27121 | ttgccctcct cccagctctg gtattgcagc ttcctcctgg ctgcaaactt tctccacaat |
| 27181 | ctaaatggaa tgtcagtttc ctcctgttcc tgtccatccg cacccactat cttcatgttg |
| 27241 | ttgcagatga agcgcgcaag accgtctgaa gataccttca accccgtgta tccatatgac |
| 27301 | acggaaaccg gtcctccaac tgtgcctttt cttactcctc cctttgtatc ccccaatggg |
| 27361 | tttcaagaga gtccccctgg ggtactctct ttgcgcctat ccgaacctct agttacctcc |
| 27421 | aatggcatgc ttgcgctcaa aatgggcaac ggcctctctc tggacgaggc cggcaacctt |
| 27481 | acctcccaaa atgtaaccac tgtgagccca cctctcaaaa aaaccaagtc aaacataaac |
| 27541 | ctggaaatat ctgcacccct cacagttacc tcagaagccc taactgtggc tgccgccgca |
| 27601 | cctctaatgg tcgcgggcaa cacactcacc atgcaatcac aggccccgct aaccgtgcac |
| 27661 | gactccaaac ttagcattgc cacccaagga cccctcacag tgtcagaagg aaagctagcc |
| 27721 | ctgcaaacat caggccccct caccaccacc gatagcagta cccttactat cactgcctca |
| 27781 | ccccctctaa ctactgccac tggtagcttg ggcattgact tgaaagagcc catttataca |
| 27841 | caaaatggaa aactaggact aaagtacggg gctcctttgc atgtaacaga cgacctaaac |
| 27901 | actttgaccg tagcaactgg tccaggtgtg actattaata atacttcctt gcaaactaaa |
| 27961 | gttactggag ccttgggttt tgattcacaa ggcaatatgc aacttaatgt agcaggagga |
| 28021 | ctaaggattg attctcaaaa cagacgcctt atacttgatg ttagttatcc gtttgatgct |
| 28081 | caaaaccaac taaatctaag actaggacag ggccctcttt ttataaactc agcccacaac |
| 28141 | ttggatatta actacaacaa aggcctttac ttgtttacag cttcaaacaa ttccaaaaag |
| 28201 | cttgaggtta acctaagcac tgccaagggg ttgatgtttg acgctacagc catagccatt |
| 28261 | aatgcaggag atgggcttga atttggttca cctaatgcac caaacacaaa tcccctcaaa |
| 28321 | acaaaaattg gccatggcct agaatttgat tcaaacaagg ctatggttcc taaactagga |
| 28381 | actggcctta gttttgacag cacaggtgcc attacagtag gaaacaaaaa taatgataag |
| 28441 | ctaactttgt ggaccacacc agctccatct cctaactgta gactaaatgc agagaaagat |
| 28501 | gctaaactca ctttggtctt aacaaaatgt ggcagtcaaa tacttgctac agtttcagtt |
| 28561 | ttggctgtta aaggcagttt ggctccaata tctggaacag ttcaaagtgc tcatcttatt |
| 28621 | ataagatttg acgaaaatgg agtgctacta aacaattcct tcctggaccc agaatattgg |
| 28681 | aactttagaa atggagatct tactgaaggc acagcctata caaacgctgt tggatttatg |
| 28741 | cctaacctat cagcttatcc aaaatctcac ggtaaaactg ccaaaagtaa cattgtcagt |
| 28801 | caagtttact taaacggaga caaaactaaa cctgtaacac taaccattac actaaacggt |
| 28861 | acacaggaaa caggagacac aactccaagt gcatactcta tgtcattttc atgggactgg |
| 28921 | tctggccaca actacattaa tgaaatattt gccacatcct cttacacttt ttcatacatt |
| 28981 | gcccaagaat aaagaatcgt ttgtgttatg tttcaacgtg tttatttttc aattgcagaa |
| 29041 | aatttcgaat catttttcat tcagtagtat agccccacca ccacatagct tatacagatc |
| 29101 | accgtacctt aatcaaactc acagaaccct agtattcaac ctgccacctc cctcccaaca |
| 29161 | cacagagtac acagtccttt ctccccggct ggccttaaaa agcatcatat catgggtaac |
| 29221 | agacatattc ttaggtgtta tattccacac ggtttcctgt cgagccaaac gctcatcagt |
| 29281 | gatattaata aactccccgg gcagctcact taagttcatg tcgctgtcca gctgctgagc |
| 29341 | cacaggctgc tgtccaactt gcggttgctt aacgggcggc gaaggagaag tccacgccta |
| 29401 | catgggggta gagtcataat cgtgcatcag gatagggcgg tggtgctgca gcagcgcgcg |
| 29461 | aataaactgc tgccgccgcc gctccgtcct gcaggaatac aacatggcag tggtctcctc |
| 29521 | agcgatgatt cgcaccgccc gcagcataag gcgccttgtc ctccgggcac agcagcgcac |
| 29581 | cctgatctca cttaaatcag cacagtaact gcagcacagc accacaatat tgttcaaaat |
| 29641 | cccacagtgc aaggcgctgt atccaaagct catggcgggg accacagaac ccacgtggcc |
| 29701 | atcataccac aagcgcaggt agattaagtg gcgacccctc ataaacacgc tggacataaa |
| 29761 | cattacctct tttggcatgt tgtaattcac cacctcccgg taccatataa acctctgatt |
| 29821 | aaacatggcg ccatccacca ccatcctaaa ccagctggcc aaaacctgcc cgccggctat |
| 29881 | acactgcagg gaaccgggac tggaacaatg acagtggaga gcccaggact cgtaaccatg |
| 29941 | gatcatcatg ctcgtcatga tatcaatgtt ggcacaacac aggcacacgt gcatacactt |
| 30001 | cctcaggatt acaagctcct cccgcgttag aaccatatcc cagggaacaa cccattcctg |
| 30061 | aatcagcgta aatcccacac tgcagggaag acctcgcacg taactcacgt tgtgcattgt |
| 30121 | caaagtgtta cattcgggca gcagcggatg atcctccagt atggtagcgc gggtttctgt |
| 30181 | ctcaaaagga ggtagacgat ccctactgta cggagtgcgc cgagacaacc gagatcgtgt |
| 30241 | tggtcgtagt gtcatgccaa atggaacgcc ggacgtagtc atatttcctg aagcaaaacc |
| 30301 | aggtgcgggc gtgacaaaca gatctgcgtc tccggtctcg ccgcttagat cgctctgtgt |
| 30361 | agtagttgta gtatatccac tctctcaaag catccaggcg ccccctggct tcgggttcta |
| 30421 | tgtaaactcc ttcatgcgcc gctgccctga taacatccac caccgcagaa taagccacac |
| 30481 | ccagccaacc tacacattcg ttctgcgagt cacacacggg aggagcggga agagctggaa |
| 30541 | gaaccatgtt ttttttttta ttccaaaaga ttatccaaaa cctcaaaatg aagatctatt |
| 30601 | aagtgaacgc gctcccctcc ggtggcgtgg tcaaactcta cagccaaaga acagataatg |
| 30661 | gcatttgtaa gatgttgcac aatggcttcc aaaaggcaaa cggccctcac gtccaagtgg |
| 30721 | acgtaaaggc taaacccttc agggtgaatc tcctctataa acattccagc accttcaacc |
| 30781 | atgcccaaat aattctcatc tcgccacctt ctcaatatat ctctaagcaa atcccgaata |
| 30841 | ttaagtccgg ccattgtaaa aatctgctcc agagcgccct ccaccttcag cctcaagcag |
| 30901 | cgaatcatga ttgcaaaaat tcaggttcct cacagacctg tataagattc aaaagcggaa |
| 30961 | cattaacaaa aataccgcga tcccgtaggt cccttcgcag ggccagctga acataatcgt |
| 31021 | gcaggtctgc acggaccagc gcggccactt ccccgccagg aaccttgaca aaagaaccca |
| 31081 | cactgattat gacacgcata ctcggagcta tgctaaccag cgtagccccg atgtaagctt |
| 31141 | tgttgcatgg gcggcgatat aaaatgcaag gtgctgctca aaaaatcagg caaagcctcg |
| 31201 | cgcaaaaaag aaagcacatc gtagtcatgc tcatgcagat aaaggcaggt aagctccgga |
| 31261 | accaccacag aaaaagacac catttttctc tcaaacatgt ctgcgggttt ctgcataaac |
| 31321 | acaaaataaa ataacaaaaa aacatttaaa cattagaagc ctgtcttaca acaggaaaaa |
| 31381 | caacccttat aagcataaga cggactacgg ccatgccggc gtgaccgtaa aaaaactggt |
| 31441 | caccgtgatt aaaaagcacc accgacagct cctcggtcat gtccggagtc ataatgtaag |
| 31501 | actcggtaaa cacatcaggt tgattcacat cggtcagtgc taaaaagcga ccgaaatagc |
| 31561 | ccgggggaat acatacccgc aggcgtagag acaacattac agcccccata ggaggtataa |
| 31621 | caaaattaat aggagagaaa aacacataaa cacctgaaaa accctcctgc ctaggcaaaa |
| 31681 | tagcaccctc ccgctccaga acaacataca gcgcttccac agcggcagcc ataacagtca |
| 31741 | gccttaccag taaaaaagaa aacctattaa aaaaacacca ctcgacacgg caccagctca |
| 31801 | atcagtcaca gtgtaaaaaa gggccaagtg cagagcgagt atatatagga ctaaaaaatg |
| 31861 | acgtaacggt taaagtccac aaaaaacacc cagaaaaccg cacgcgaacc tacgcccaga |
| 31921 | aacgaaagcc aaaaaaccca caacttcctc aaatcgtcac ttccgttttc ccacgttacg |
| 31981 | tcacttccca ttttaagaaa actacaattc ccaacacata caagttactc cgccctaaaa |
| 32041 | cctacgtcac ccgccccgtt cccacgcccc gcgccacgtc acaaactcca ccccctcatt |
| 32101 | atcatattgg cttcaatcca aaataaggta tattattgat gatgttaatt aatttaaatc |
| 32161 | cgcatgcgat atcgagctct cccgggaatt cggatctgcg acgcgaggct ggatggcctt |
| 32221 | ccccattatg attcttctcg cttccggcgg catcgggatg cccgcgttgc aggccatgct |
| 32281 | gtccaggcag gtagatgacg accatcaggg acagcttcac ggccagcaaa aggccaggaa |
| 32341 | ccgtaaaaag gccgcgttgc tggcgttttt ccataggctc cgcccccctg acgagcatca |
| 32401 | caaaaatcga cgctcaagtc agaggtggcg aaacccgaca ggactataaa gataccaggc |
| 32461 | gtttccccct ggaagctccc tcgtgcgctc tcctgttccg accctgccgc ttaccggata |
| 32521 | cctgtccgcc tttctccctt cgggaagcgt ggcgctttct caatgctcac gctgtaggta |
| 32581 | tctcagttcg gtgtaggtcg ttcgctccaa gctgggctgt gtgcacgaac cccccgttca |
| 32641 | gcccgaccgc tgcgccttat ccggtaacta tcgtcttgag tccaacccgg taagacacga |
| 32701 | cttatcgcca ctggcagcag ccactggtaa caggattagc agagcgaggt atgtaggcgg |
| 32761 | tgctacagag ttcttgaagt ggtggcctaa ctacggctac actagaagga cagtatttgg |
| 32821 | tatctgcgct ctgctgaagc cagttacctt cggaaaaaga gttggtagct cttgatccgg |
| 32881 | caaacaaacc accgctggta gcggtggttt ttttgtttgc aagcagcaga ttacgcgcag |
| 32941 | aaaaaaagga tctcaagaag atcctttgat cttttctacg gggtctgacg ctcagtggaa |
| 33001 | cgaaaactca cgttaaggga ttttggtcat gagattatca aaaaggatct tcacctagat |
| 33061 | ccttttaaat caatctaaag tatatatgag taaacttggt ctgacagtta ccaatgctta |
| 33121 | atcagtgagg cacctatctc agcgatctgt ctatttcgtt catccatagt tgcctgactc |
| 33181 | cccgtcgtgt agataactac gatacgggag ggcttaccat ctggccccag tgctgcaatg |
| 33241 | ataccgcgag acccacgctc accggctcca gatttatcag caataaacca gccagccgga |
| 33301 | agggccgagc gcagaagtgg tcctgcaact ttatccgcct ccatccagtc tattaattgt |
| 33361 | tgccgggaag ctagagtaag tagttcgcca gttaatagtt tgcgcaacgt tgttgccatt |
| 33421 | gntgcaggca tcgtggtgtc acgctcgtcg tttggtatgg cttcattcag ctccggttcc |
| 33481 | caacgatcaa ggcgagttac atgatccccc atgttgtgca aaaaagcggt tagctccttc |
| 33541 | ggtcctccga tcgttgtcag aagtaagttg gccgcagtgt tatcactcat ggttatggca |
| 33601 | gcactgcata attctcttac tgtcatgcca tccgtaagat gcttttctgt gactggtgag |
| 33661 | tactcaacca agtcattctg agaatagtgt atgcggcgac cgagttgctc ttgcccggcg |
| 33721 | tcaacacggg ataataccgc gccacatagc agaactttaa aagtgctcat cattggaaaa |
| 33781 | cgttcttcgg ggcgaaaact ctcaaggatc ttaccgctgt tgagatccag ttcgatgtaa |
| 33841 | cccactcgtg cacccaactg atcttcagca tcttttactt tcaccagcgt ttctgggtga |
| 33901 | gcaaaaacag gaaggcaaaa tgccgcaaaa aagggaataa gggcgacacg gaaatgttga |
| 33961 | atactcatac tcttcctttt tcaatattat tgaagcattt atcagggtta ttgtctcatg |
| 34021 | agcggataca tatttgaatg tatttagaaa aataaacaaa taggggttcc gcgcacattt |
| 34081 | ccccgaaaag tgccacctga cgtctaagaa accattatta tcatgacatt aacctataaa |
| 34141 | aataggcgta tcacgaggcc ctttcgtctt caaggatccg aattcccggg agagctcgat |
| 34201 | atcgcatgcg gatttaaatt aattaa |

TABLE 9

Nucleotide sequence of a Sau3A fragment used to construct
vectors comprising suppressor tRNA sequences.

| | | | | | | |
|---|---|---|---|---|---|---|
| 1 | ctagaggatc | gaaaccatcc | tctgctatat | ggccgcatat | attttacttg | aagactagga |
| 61 | ccctacagaa | aaggggtttt | aaagtaggcg | tgctaaacgt | cagcggacct | gacccgtgta |
| 121 | agaatccaca | aggtatcctg | gtggaaatgc | gcatttgtag | gcttcaatat | ctgtaatcct |
| 181 | actaattagg | tgtggagagc | tttcagccag | tttcgtaggt | ttggagacca | tttaggggtt |
| 241 | ggcgtgtggc | cccctcgtaa | agtctttcgt | acttcctaca | tcagacaagt | cttgcaattt |
| 301 | gcaatatctc | ttttagccaa | tatctaaatc | tttaaaattt | tgattttgtt | ttttaaccag |
| 361 | gatgagagac | attccagagt | tgttaccttg | tcaaaataaa | caaatttaaa | gatgtctgtg |
| 421 | aaaagaaaca | tatattcctc | atgggaatat | atccaggttg | ttgaaggagg | tacactcgag |
| 481 | tctccctatc | agtgatagag | atctcgaggt | cgtagtcgtg | gccgagtggt | taaggcgatg |
| 541 | gactctaaat | ccattggggt | ctccccgcgc | aggttcgaat | cctgccgact | acggcgtgct |
| 601 | ttttttactc | tcgggtagag | gaaatccggt | gcactacctg | tgcaatcaca | cagaataaca |
| 661 | tggagtagta | ctttttattt | tcctgttatt | atctttctcc | ataaaagtgg | aaccagataa |
| 721 | ttttagttct | tttgtgtaac | aagactagag | attttttgaa | gtgttacatt | ggaaagcact |
| 781 | tgaaaacaca | agtaatttct | gacactgcta | taaaaatgat | ggaaaaacgc | tcaagttgtt |
| 841 | ttgcctttca | gtcttcttga | aatgctgtct | ccctatctga | aatccagctc | acgtctgact |
| 901 | tccaaaaccg | tgcttgcctt | taacttatgg | aataaatatc | tcaaacagat | cccc |

TABLE 10
Nucleotide sequence of pAd/PL-DEST ™.

CATCATCAATAATATACCTTATTTTGGATTGAAGCCAATATGATAATGAGGGGGTGGAGTTTGTGACGTG
GCGCGGGGCGTGGGAACGGGGCGGGTGACGTAGTAGTGTGGCGGAAGTGTGATGTTGCAAGTGTGGCGGA
ACACATGTAAGCGACGGATGTGGCAAAAGTGACGTTTTTGGTGTGCGCCGGTGTACACAGGAAGTGACAA
TTTTCGCGCGGTTTTAGGCGGATGTTGTAGTAAATTTGGGCGTAACCGAGTAAGATTTGGCCATTTTCGC
GGGAAAACTGAATAAGAGGAAGTGAAATCTGAATAATTTTGTGTTACTCATAGCGCGTAATATTTGTCTA
GGGCCGCGGGGACTTTGACCGTTTACGTGGAGACTCGCCCAGGTGTTTTTCTCAGGTGTTTTCCGCGTTC
CGGGTCAAAGTTGGCGTTTTATTATTATAGTCAGTCGAAGCTTGGATCCGGTACCTCTAGAATTCTCGAG
CGGCCGCTAGCGACATCGATCACAAGTTTGTACAAAAAAGCTGAACGAGAAACGTAAAATGATATAAATA
TCAATATATTAAATTAGATTTTGCATAAAAAACAGACTACATAATACTGTAAAACACAACATATCCAGTC
ACTATGGCGGCCGCATTAGGCACCCCAGGCTTTACACTTTATGCTTCCGGCTCGTATAATGTGTGGATTT
TGAGTTAGGATCCGGCGAGATTTTCAGGAGCTAAGGAAGCTAAAATGGAGAAAAAAATCACTGGATATAC
CACCGTTGATATATCCCAATGGCATCGTAAAGAACATTTTGAGGCATTTCAGTCAGTTGCTCAATGTACC
TATAACCAGACCGTTCAGCTGGATATTACGGCCTTTTTAAAGACCGTAAAGAAAAATAAGCACAAGTTTT
ATCCGGCCTTTATTCACATTCTTGCCCGCCTGATGAATGCTCATCCGGAATTCCGTATGGCAATGAAAGA
CGGTGAGCTGGTGATATGGGATAGTGTTCACCCTTGTTACACCGTTTTCCATGAGCAAACTGAAACGTTT
TCATCGCTCTGGAGTGAATACCACGACGATTTCCGGCAGTTTCTACACATATATTCGCAAGATGTGGCGT
GTTACGGTGAAAACCTGGCCTATTTCCCTAAAGGGTTTATTGAGAATATGTTTTTCGTCTCAGCCAATCC
CTGGGTGAGTTTCACCAGTTTTGATTTAAACGTGGCCAATATGGACAACTTCTTCGCCCCCGTTTTCACC
ATGGGCAAATATTATACGCAAGGCGACAAGGTGCTGATGCCGCTGGCGATTCAGGTTCATCATGCCGTCT
GTGATGGCTTCCATGTCGGCAGAATGCTTAATGAATTACAACAGTACTGCGATGAGTGGCAGGGCGGGGC
GTAAACGCGTGGATCCGGCTTACTAAAAGCCAGATAACAGTATGCGTATTTGCGCGCTGATTTTTGCGGT
ATAAGAATATATACTGATATGTATACCCGAAGTATGTCAAAAAGAGGTGTGCTATGAAGCAGCGTATTAC
AGTGACAGTTGACAGCGACAGCTATCAGTTGCTCAAGGCATATATGATGTCAATATCTCCGGTCTGGTAA
GCACAACCATGCAGAATGAAGCCCGTCGTCTGCGTGCCGAACGCTGGAAAGCGGAAAATCAGGAAGGGAT
GGCTGAGGTCGCCCGGTTTATTGAAATGAACGGCTCTTTTGCTGACGAGAACAGGGACTGGTGAAATGCA
GTTTAAGGTTTACACCTATAAAAGAGAGAGCCGTTATCGTCTGTTTGTGGATGTACAGAGTGATATTATT
GACACGCCCGGGCGACGGATGGTGATCCCCCTGGCCAGTGCACGTCTGCTGTCAGATAAAGTCTCCCGTG
AACTTTACCCGGTGGTGCATATCGGGGATGAAAGCTGGCGCATGATGACCACCGATATGGCCAGTGTGCC
GGTCTCCGTTATCGGGGAAGAAGTGGCTGATCTCAGCCACCGCGAAAATGACATCAAAAACGCCATTAAC
CTGATGTTCTGGGGAATATAAATGTCAGGCTCCGTTATACACAGCCAGTCTGCAGGTCGACCATAGTGAC
TGGATATGTTGTGTTTTACAGTATTATGTAGTCTGTTTTTTATGCAAAATCTAATTTAATATATTGATAT
TTATATCATTTTACGTTTCTCGTTCAGCTTTCTTGTACAAAGTGGTGATCGATTCGACAGATCACTGAAA
TGTGTGGGCGTGGCTTAAGGGTGGGAAAGAATATATAAGGTGGGGGTCTTATGTAGTTTTGTATCTGTTT
TGCAGCAGCCGCCGCCGCCATGAGCACCAACTCGTTTGATGGAAGCATTGTGAGCTCATATTTGACAACG
CGCATGCCCCCATGGGCCGGGGTGCGTCAGAATGTGATGGGCTCCAGCATTGATGGTCGCCCCGTCCTGC
CCGCAAACTCTACTACCTTGACCTACGAGACCGTGTCTGGAACGCCGTTGGAGACTGCAGCCTCCGCCGC
CGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGA
CCCGGGAACTTAATGTCGTTTCTCAGCAGCTGTTGGATCTGCGCCAGCAGGTTTCTGCCCTGAAGGCTTC
CTCCCCTCCCAATGCGGTTTAAAACATAAATAAAAAACCAGACTCTGTTTGGATTTGGATCAAGCAAGTG
TCTTGCTGTCTTTATTTAGGGGTTTTGCGCGCGCGGTAGGCCCGGGACCAGCGGTCTCGGTCGTTGAGGG
TCCTGTGTATTTTTTCCAGGACGTGGTAAAGGTGACTCTGGATGTTCAGATACATGGGCATAAGCCCGTC
TCTGGGGTGGAGGTAGCACCACTGCAGAGCTTCATGCTGCGGGGTGGTGTTGTAGATGATCCAGTCGTAG
CAGGAGCGCTGGGCGTGGTGCCTAAAAATGTCTTTCAGTAGCAAGCTGATTGCCAGGGGCAGGCCCTTGG
TGTAAGTGTTTACAAAGCGGTTAAGCTGGGATGGGTGCATACGTGGGGATATGAGATGCATCTTGGACTG
TATTTTTAGGTTGGCTATGTTCCCAGCCATATCCCTCCGGGGATTCATGTTGTGCAGAACCACCAGCACA
GTGTATCCGGTGCACTTGGGAAATTTGTCATGTAGCTTAGAAGGAAATGCGTGGAAGAACTTGGAGACGC
CCTTGTGACCTCCAAGATTTTCCATGCATTCGTCCATAATGATGGCAATGGGCCCACGGGCGGCGGCCTG
GGCGAAGATATTTCTGGGATCACTAACGTCATAGTTGTGTTCCAGGATGAGATCGTCATAGGCCATTTTT
ACAAAGCGCGGGCGGAGGGTGCCAGACTGCGGTATAATGGTTCCATCCGGCCCAGGGGCGTAGTTACCCT
CACAGATTTGCATTTCCCACGCTTTGAGTTCAGATGGGGGGATCATGTCTACCTGCGGGGCGATGAAGAA
AACGGTTTCCGGGGTAGGGGAGATCAGCTGGGAAGAAAGCAGGTTCCTGAGCAGCTGCGACTTACCGCAG
CCGGTGGGCCCGTAAATCACACCTATTACCGGGTGCAACTGGTAGTTAAGAGAGCTGCAGCTGCCGTCAT
CCCTGAGCAGGGGGGCCACTTCGTTAAGCATGTCCCTGACTCGCATGTTTTCCCTGACCAAATCCGCCAG
AAGGCGCTCGCCGCCCAGCGATAGCAGTTCTTGCAAGGAAGCAAAGTTTTTCAACGGTTTGAGACCGTCC
GCCGTAGGCATGCTTTTGAGCGTTTGACCAAGCAGTTCCAGGCGGTCCCACAGCTCGGTCACCTGCTCTA
CGGCATCTCGATCCAGCATATCTCCTCGTTTCGCGGGTTGGGGCGGCTTTCGCTGTACGGCAGTAGTCGG
TGCTCGTCCAGACGGGCCAGGGTCATGTCTTTCCACGGGCGCAGGGTCCTCGTCAGCGTAGTCTGGGTCA
CGGTGAAGGGGTGCGCTCCGGGCTGCGCGCTGGCCAGGGTGCGCTTGAGGCTGGTCCTGCTGGTGCTGAA
GCGCTGCCGGTCTTCGCCCTGCGCGTCGGCCAGGTAGCATTTGACCATGGTGTCATAGTCCAGCCCCTCC
GCGGCGTGGCCCTTGGCGCGCAGCTTGCCCTTGGAGGAGGCGCCGCACGAGGGGCAGTGCAGACTTTTGA
GGGCGTAGAGCTTGGGCGCGAGAAATACCGATTCCGGGGAGTAGGCATCCGCGCCGCAGGCCCCGCAGAC
GGTCTCGCATTCCACGAGCCAGGTGAGCTCTGGCCGTTCGGGGTCAAAAACCAGGTTTCCCCCATGCTTT
TTGATGCGTTTCTTACCTCTGGTTTCCATGAGCCGGTGTCCACGCTCGGTGACGAAAAGGCTGTCCGTGT
CCCCGTATACAGACTTGAGAGGCCTGTCCTCGAGCGGTGTTCCGCGGTCCTCCTCGTATAGAAACTCGGA
CCACTCTGAGACAAAGGCTCGCGTCCAGGCCAGCACGAAGGAGGCTAAGTGGGAGGGGTAGCGGTCGTTG
TCCACTAGGGGGTCCACTCGCTCCAGGGTGTGAAGACACATGTCGCCCTCTTCGGCATCAAGGAAGGTGA
TTGGTTTGTAGGTGTAGGCCACGTGACCGGGTGTTCCTGAAGGGGGGCTATAAAAGGGGGTGGGGGCGCG
TTCGTCCTCACTCTCTTCCGCATCGCTGTCTGCGAGGGCCAGCTGTTGGGGTGAGTACTCCCTCTGAAAA
GCGGGCATGACTTCTGCGCTAAGATTGTCAGTTTCCAAAAACGAGGAGGATTTGATATTCACCTGGCCCG
CGGTGATGCCTTTGAGGGTGGCCGCATCCATCTGGTCAGAAAAGACAATCTTTTTGTTGTCAAGCTTGGT
GGCAAACGACCCGTAGAGGGCGTTGGACAGCAACTTGGCGATGGAGCGCAGGGTTTGGTTTTTGTCGCGA
TCGGCGCGCTCCTTGGCCGCGATGTTTAGCTGCACGTATTCGCGCGCAACGCACCGCCATTCGGGAAAGA
CGGTGGTGCGCTCGTCGGGCACCAGGTGCACGCGCCAACCGCGGTTGTGCAGGGTGACAAGGTCAACGCT
GGTGGCTACCTCTCCGCGTAGGCGCTCGTTGGTCCAGCAGAGGCGGCCGCCCTTGCGCGAGCAGAATGGC
GGTAGGGGGTCTAGCTGCGTCTCGTCCGGGGGGTCTGCGTCCACGGTAAAGACCCCGGGCAGCAGGCGCG
CGTCGAAGTAGTCTATCTTGCATCCTTGCAAGTCTAGCGCCTGCTGCCATGCGCGGGCGGCAAGCGCGCG
CTCGTATGGGTTGAGTGGGGGACCCCATGGCATGGGGTGGGTGAGCGCGGAGGCGTACATGCCGCAAATG
TCGTAAACGTAGAGGGGCTCTCTGAGTATTCCAAGATATGTAGGGTAGCATCTTCCACCGCGGATGCTGG
CGCGCACGTAATCGTATAGTTCGTGCGAGGGAGCGAGGAGGTCGGGACCGAGGTTGCTACGGGCGGGCTG
CTCTGCTCGGAAGACTATCTGCCTGAAGATGGCATGTGAGTTGGATGATATGGTTGGACGCTGGAAGACG
TTGAAGCTGGCGTCTGTGAGACCTACCGCGTCACGCACGAAGGAGGCGTAGGAGTCGCGCAGCTTGTTGA
CCAGCTCGGCGGTGACCTGCACGTCTAGGGCGCAGTAGTCCAGGGTTTCCTTGATGATGTCATACTTATC
CTGTCCCTTTTTTTTCCACAGCTCGCGGTTGAGGACAAACTCTTCGCGGTCTTTCCAGTACTCTTGGATC
GGAAACCCGTCGGCCTCCGAACGGTAAGAGCCTAGCATGTAGAACTGGTTGACGGCCTGGTAGGCGCAGC
ATCCCTTTTCTACGGGTAGCGCGTATGCCTGCGCGGCCTTCCGGAGCGAGGTGTGGGTGAGCGCAAAGGT
GTCCCTGACCATGACTTTGAGGTACTGGTATTTGAAGTCAGTGTCGTCGCATCCGCCCTGCTCCCAGAGC
AAAAAGTCCGTGCGCTTTTTGGAACGCGGATTTGGCAGGGCGAAGGTGACATCGTTGAAGAGTATCTTTC
CCGCGCGAGGCATAAAGTTGCGTGTGATGCGGAAGGGTCCCGGCACCTCGGAACGGTTGTTAATTACCTG
GGCGGCGAGCACGATCTCGTCAAAGCCGTTGATGTTGTGGCCCACAATGTAAAGTTCCAAGAAGCGCGGG
ATGCCCTTGATGGAAGGCAATTTTTTAAGTTCCTCGTAGGTGAGCTCTTCAGGGGAGCTGAGCCCGTGCT
CTGAAAGGGCCCAGTCTGCAAGATGAGGGTTGGAAGCGACGAATGAGCTCCACAGGTCACGGGCCATTAG
CATTTGCAGGTGGTCGCGAAAGGTCCTAAACTGGCGACCTATGGCCATTTTTTCTGGGGTGATGCAGTAG
AAGGTAAGCGGGTCTTGTTCCCAGCGGTCCCATCCAAGGTTCGCGGCTAGGTCTCGCGCGGCAGTCACTA
GAGGCTCATCTCCGCCGAACTTCATGACCAGCATGAAGGGCACGAGCTGCTTCCCAAAGGCCCCCATCCA
AGTATAGGTCTCTACATCGTAGGTGACAAAGAGACGCTCGGTGCGAGGATGCGAGCCGATCGGGAAGAAC
TGGATCTCCCGCCACCAATTGGAGGAGTGGCTATTGATGTGGTGAAAGTAGAAGTCCCTGCGACGGGCCG
AACACTCGTGCTGGCTTTTGTAAAAACGTGCGCAGTACTGGCAGCGGTGCACGGGCTGTACATCCTGCAC
GAGGTTGACCTGACGACCGCGCACAAGGAAGCAGAGTGGGAATTTGAGCCCCTCGCCTGGCGGGTTTGGC
TGGTGGTCTTCTACTTCGGCTGCTTGTCCTTGACCGTCTGGCTGCTCGAGGGGAGTTACGGTGGATCGGA
CCACCACGCCGCGCGAGCCCAAAGTCCAGATGTCCGCGCGCGGCGGTCGGAGCTTGATGACAACATCGCG
CAGATGGGAGCTGTCCATGGTCTGGAGCTCCCGCGGCGTCAGGTCAGGCGGGAGCTCCTGCAGGTTTACC
TCGCATAGACGGGTCAGGGCGCGGGCTAGATCCAGGTGATACCTAATTTCCAGGGGCTGGTTGGTGGCGG
CGTCGATGGCTTGCAAGAGGCCGCATCCCCGCGGCGCGACTACGGTACCGCGCGGCGGGCGGTGGGCCGC
GGGGGTGTCCTTGGATGATGCATCTAAAAGCGGTGACGCGGGCGAGCCCCCGGAGGTAGGGGGGGCTCCG
GACCCGCCGGGAGAGGGGGCAGGGGCACGTCGGCGCCGCGCGCGGGCAGGAGCTGGTGCTGCGCGCGTAG
GTTGCTGGCGAACGCGACGACGCGGCGGTTGATCTCCTGAATCTGGCGCCTCTGCGTGAAGACGACGGGC
CCGGTGAGCTTGAGCCTGAAAGAGAGTTCGACAGAATCAATTTCGGTGTCGTTGACGGCGGCCTGGCGCA
AAATCTCCTGCACGTCTCCTGAGTTGTCTTGATAGGCGATCTCGGCCATGAACTGCTCGATCTCTTCCTC
CTGGAGATCTCCGCGTCCGGCTCGCTCCACGGTGGCGGCGAGGTCGTTGGAAATGCGGGCCATGAGCTGC
GAGAAGGCGTTGAGGCCTCCCTCGTTCCAGACGCGGCTGTAGACCACGCCCCCTTCGGCATCGCGGGCGC
GCATGACCACCTGCGCGAGATTGAGCTCCACGTGCCGGGCGAAGACGGCGTAGTTTCGCAGGCGCTGAAA
GAGGTAGTTGAGGGTGGTGGCGGTGTGTTCTGCCACGAAGAAGTACATAACCCAGCGTCGCAACGTGGAT
TCGTTGATATCCCCCAAGGCCTCAAGGCGCTCCATGGCCTCGTAGAAGTCCACGGCGAAGTTGAAAAACT
GGGAGTTGCGCGCCGACACGGTTAACTCCTCCTCCAGAAGACGGATGAGCTCGGCGACAGTGTCGCGCAC
CTCGCGCTCAAAGGCTACAGGGGCCTCTTCTTCTTCTTCAATCTCCTCTTCCATAAGGGCCTCCCCTTCT
TCTTCTTCTGGCGGCGGTGGGGGAGGGGGGACACGGCGGCGACGACGGCGCACCGGGAGGCGGTCGACAA
AGCGCTCGATCATCTCCCCGCGGCGACGGCGCATGGTCTCGGTGACGGCGCGGCCGTTCTCGCGGGGGCG
CAGTTGGAAGACGCCGCCCGTCATGTCCCGGTTATGGGTTGGCGGGGGGCTGCCATGCGGCAGGGATACG
GCGCTAACGATGCATCTCAACAATTGTTGTGTAGGTACTCCGCCGCCGAGGGACCTGAGCGAGTCCGCAT
CGACCGGATCGGAAAACCTCTCGAGAAAGGCGTCTAACCAGTCACAGTCGCAAGGTAGGCTGAGCACCGT
GGCGGGCGGCAGCGGGCGGCGGTCGGGGTTGTTTCTGGCGGAGGTGCTGCTGATGATGTAATTAAAGTAG
GCGGTCTTGAGACGGCGGATGGTCGACAGAAGCACCATGTCCTTGGGTCCGGCCTGCTGAATGCGCAGGC
GGTCGGCCATGCCCCAGGCTTCGTTTTGACATCGGCGCAGGTCTTTGTAGTAGTCTTGCATGAGCCTTTC
TACCGGCACTTCTTCTTCTCCTTCCTCTTGTCCTGCATCTCTTGCATCTATCGCTGCGGCGGCGGCGGAG
TTTGGCCGTAGGTGGCGCCCTCTTCCTCCCATGCGTGTGACCCCGAAGCCCCTCATCGGCTGAAGCAGGG
CTAGGTCGGCGACAACGCGCTCGGCTAATATGGCCTGCTGCACCTGCGTGAGGGTAGACTGGAAGTCATC
CATGTCCACAAAGCGGTGGTATGCGCCCGTGTTGATGGTGTAAGTGCAGTTGGCCATAACGGACCAGTTA
ACGGTCTGGTGACCCGGCTGCGAGAGCTCGGTGTACCTGAGACGCGAGTAAGCCCTCGAGTCAAATACGT
AGTCGTTGCAAGTCCGCACCAGGTACTGGTATCCCACCAAAAAGTGCGGCGGCGGCTGGCGGTAGAGGGG
CCAGCGTAGGGTGGCCGGGGCTCCGGGGGCGAGATCTTCCAACATAAGGCGATGATATCCGTAGATGTAC
CTGGACATCCAGGTGATGCCGGCGGCGGTGGTGGAGGCGCGCGGAAAGTCGCGGACGCGGTTCCAGATGT
TGCGCAGCGGCAAAAAGTGCTCCATGGTCGGGACGCTCTGGCCGGTCAGGCGCGCGCAATCGTTGACGCT
CTAGACCGTGCAAAAGGAGAGCCTGTAAGCGGGCACTCTTCCGTGGTCTGGTGGATAAATTCGCAAGGGT
ATCATGGCGGACGACCGGGGTTCGAGCCCCGTATCCGGCCGTCCGCCGTGATCCATGCGGTTACCGCCCG
CGTGTCGAACCCAGGTGTGCGACGTCAGACAACGGGGGAGTGCTCCTTTTGGCTTCCTTCCAGGCGCGGC
GGCTGCTGCGCTAGCTTTTTTGGCCACTGGCCGCGCGCAGCGTAAGCGGTTAGGCTGGAAAGCGAAAGCA
TTAAGTGGCTCGCTCCCTGTAGCCGGAGGGTTATTTTCCAAGGGTTGAGTCGCGGGACCCCCGGTTCGAG
TCTCGGACCGGCCGGACTGCGGCGAACGGGGGTTTGCCTCCCCGTCATGCAAGACCCCGCTTGCAAATTC
CTCCGGAAACAGGGACGAGCCCCTTTTTTGCTTTTCCCAGATGCATCCGGTGCTGCGGCAGATGCGCCCC
CCTCCTCAGCAGCGGCAAGAGCAAGAGCAGCGGCAGACATGCAGGGCACCCTCCCCTCCTCCTACCGCGT
CAGGAGGGGCGACATCCGCGGTTGACGCGGCAGCAGATGGTGATTACGAACCCCCGCGGCGCCGGGCCCG
GCACTACCTGGACTTGGAGGAGGGCGAGGGCCTGGCGCGGCTAGGAGCGCCCTCTCCTGAGCGGTACCCA
AGGGTGCAGCTGAAGCGTGATACGCGTGAGGCGTACGTGCCGCGGCAGAACCTGTTTCGCGACCGCGAGG
GAGAGGAGCCCGAGGAGATGCGGGATCGAAAGTTCCACGCAGGGCGCGAGCTGCGGCATGGCCTGAATCG
CGAGCGGTTGCTGCGCGAGGAGGACTTTGAGCCCGACGCGCGAACCGGGATTAGTCCCGCGCGCGCACAC
GTGGCGGCCGCCGACCTGGTAACCGCATACGAGCAGACGGTGAACCAGGAGATTAACTTTCAAAAAAGCT
TTAACAACCACGTGCGTACGCTTGTGGCGCGCGAGGAGGTGGCTATAGGACTGATGCATCTGTGGGACTT
TGTAAGCGCGCTGGAGCAAAACCCAAATAGCAAGCCGCTCATGGCGCAGCTGTTCCTTATAGTGCAGCAC
AGCAGGGACAACGAGGCATTCAGGGATGCGCTGCTAAACATAGTAGAGCCCGAGGGCCGCTGGCTGCTCG
ATTTGATAAACATCCTGCAGAGCATAGTGGTGCAGGAGCGCAGCTTGAGCCTGGCTGACAAGGTGGCCGC
CATCAACTATTCCATGCTTAGCCTGGGCAAGTTTTACGCCCGCAAGATATACCATACCCCTTACGTTCCC
ATAGACAAGGAGGTAAAGATCGAGGGGTTCTACATGCGCATGGCGCTGAAGGTGCTTACCTTGAGCGACG
ACCTGGGCGTTTATCGCAACGAGCGCATCCACAAGGCCGTGAGCGTGAGCCGGCGGCGCGAGCTCAGCGA
CCGCGAGCTGATGCACAGCCTGCAAAGGGCCCTGGCTGGCACGGGCAGCGGCGATAGAGAGGCCGAGTCC
TACTTTGACGCGGGCGCTGACCTGCGCTGGGCCCCAAGCCGACGCGCCCTGGAGGCAGCTGGGGCCGGAC
CTGGGCTGGCGGTGGCACCCGCGCGCGCTGGCAACGTCGGCGGCGTGGAGGAATATGACGAGGACGATGA
GTACGAGCCAGAGGACGGCGAGTACTAAGCGGTGATGTTTCTGATCAGATGATGCAAGACGCAACGGACC
CGGCGGTGCGGGCGGCGCTGCAGAGCCAGCCGTCCGGCCTTAACTCCACGGACGACTGGCGCCAGGTCAT
GGACCGCATCATGTCGCTGACTGCGCGCAATCCTGACGCGTTCCGGCAGCAGCCGCAGGCCAACCGGCTC
TCCGCAATTCTGGAAGCGGTGGTCCCGGCGCGCGCAAACCCCACGCACGAGAAGGTGCTGGCGATCGTAA
ACGCGCTGGCCGAAAACAGGGCCATCCGGCCCGACGAGGCCGGCCTGGTCTACGACGCGCTGCTTCAGCG
CGTGGCTCGTTACAACAGCGGCAACGTGCAGACCAACCTGGACCGGCTGGTGGGGGATGTGCGCGAGGCC
GTGGCGCAGCGTGAGCGCGCGCAGCAGCAGGGCAACCTGGGCTCCATGGTTGCACTAAACGCCTTCCTGA
GTACACAGCCCGCCAACGTGCCGCGGGGACAGGAGGACTACACCAACTTTGTGAGCGCACTGCGGCTAAT
GGTGACTGAGACACCGCAAAGTGAGGTGTACCAGTCTGGGCCAGACTATTTTTTCCAGACCAGTAGACAA
GGCCTGCAGACCGTAAACCTGAGCCAGGCTTTCAAAAACTTGCAGGGGCTGTGGGGGGTGCGGGCTCCCA
CAGGCGACCGCGCGACCGTGTCTAGCTTGCTGACGCCCAACTCGCGCCTGTTGCTGCTGCTAATAGCGCC
CTTCACGGACAGTGGCAGCGTGTCCCGGGACACATACCTAGGTCACTTGCTGACACTGTACCGCGAGGCC
ATAGGTCAGGCGCATGTGGACGAGCATACTTTCCAGGAGATTACAAGTGTCAGCCGCGCGCTGGGGCAGG
AGGACACGGGCAGCCTGGAGGCAACCCTAAACTACCTGCTGACCAACCGGCGGCAGAAGATCCCCTCGTT
GCACAGTTTAAACAGCGAGGAGGAGCGCATTTTGCGCTACGTGCAGCAGAGCGTGAGCCTTAACCTGATG
CGCGACGGGGTAACGCCCAGCGTGGCGCTGGACATGACCGCGCGCAACATGGAACCGGGCATGTATGCCT
CAAACCGGCCGTTTATCAACCGCCTAATGGACTACTTGCATCGCGCGGCCGCCGTGAACCCCGAGTATTT
CACCAATGCCATCTTGAACCCGCACTGGCTACCGCCCCCTGGTTTCTACACCGGGGGATTCGAGGTGCCC
GAGGGTAACGATGGATTCCTCTGGGACGACATAGACGACAGCGTGTTTTCCCCGCAACCGCAGACCCTGC
TAGAGTTGCAACAGCGCGAGCAGGCAGAGGCGGCGCTGCGAAAGGAAAGCTTCCGCAGGCCAAGCAGCTT
GTCCGATCTAGGCGCTGCGGCCCCGCGGTCAGATGCTAGTAGCCCATTTCCAAGCTTGATAGGGTCTCTT
ACCAGCACTCGCACCACCCGCCCGCGCCTGCTGGGCGAGGAGGAGTACCTAAACAACTCGCTGCTGCAGC
CGCAGCGCGAAAAAAACCTGCCTCCGGCATTTCCCAACAACGGGATAGAGAGCCTAGTGGACAAGATGAG
TAGATGGAAGACGTACGCGCAGGAGCACAGGGACGTGCCAGGCCCGCGCCCGCCCACCCGTCGTCAAAGG
CACGACCGTCAGCGGGGTCTGGTGTGGGAGGACGATGACTCGGCAGACGACAGCAGCGTCCTGGATTTGG
GAGGGAGTGGCAACCCGTTTGCGCACCTTCGCCCCAGGCTGGGGAGAATGTTTTAAAAAAAAAAAAGCAT
GATGCAAAATAAAAAACTCACCAAGGCCATGGCACCGAGCGTTGGTTTTCTTGTATTCCCCTTAGTATGC
GGCGCGCGGCGATGTATGAGGAAGGTCCTCCTCCCTCCTACGAGAGTGTGGTGAGCGCGGCGCCAGTGGC
GGCGGCGCTGGGTTCTCCCTTCGATGCTCCCCTGGACCCGCCGTTTGTGCCTCCGCGGTACCTGCGGCCT
ACCGGGGGGAGAAACAGCATCCGTTACTCTGAGTTGGCACCCCTATTCGACACCACCCGTGTGTACCTGG
TGGACAACAAGTCAACGGATGTGGCATCCCTGAACTACCAGAACGACCACAGCAACTTTCTGACCACGGT
CATTCAAAACAATGACTACAGCCCGGGGGAGGCAAGCACACAGACCATCAATCTTGACGACCGGTCGCAC
TGGGGCGGCGACCTGAAAACCATCCTGCATACCAACATGCCAAATGTGAACGAGTTCATGTTTACCAATA
AGTTTAAGGCGCGGGTGATGGTGTCGCGCTTGCCTACTAAGGACAATCAGGTGGAGCTGAAATACGAGTG
GGTGGAGTTCACGCTGCCCGAGGGCAACTACTCCGAGACCATGACCATAGACCTTATGAACAACGCGATC
GTGGAGCACTACTTGAAAGTGGGCAGACAGAACGGGGTTCTGGAAAGCGACATCGGGGTAAAGTTTGACA
CCCGCAACTTCAGACTGGGGTTTGACCCCGTCACTGGTCTTGTCATGCCTGGGGTATATACAAACGAAGC
CTTCCATCCAGACATCATTTTGCTGCCAGGATGCGGGGTGGACTTCACCCACAGCCGCCTGAGCAACTTG
TTGGGCATCCGCAAGCGGCAACCCTTCCAGGAGGGCTTTAGGATCACCTACGATGATCTGGAGGGTGGTA
ACATTCCCGCACTGTTGGATGTGGACGCCTACCAGGCGAGCTTGAAAGATGACACCGAACAGGGCGGGGG
TGGCGCAGGCGGCAGCAACAGCAGTGGCAGCGGCGCGGAAGAGAACTCCAACGCGGCAGCCGCGGCAATG
CAGCCGGTGGAGGACATGAACGATCATGCCATTCGCGGCGACACCTTTGCCACACGGGCTGAGGAGAAGC
GCGCTGAGGCCGAAGCAGCGGCCGAAGCTGCCGCCCCCGCTGCGCAACCCGAGGTCGAGAAGCCTCAGAA
GAAACCGGTGATCAAACCCCTGACAGAGGACAGCAAGAAACGCAGTTACAACCTAATAAGCAATGACAGC
ACCTTCACCCAGTACCGCAGCTGGTACCTTGCATACAACTACGGCGACCCTCAGACCGGAATCCGCTCAT
GGACCCTGCTTTGCACTCCTGACGTAACCTGCGGCTCGGAGCAGGTCTACTGGTCGTTGCCAGACATGAT
GCAAGACCCCGTGACCTTCCGCTCCACGCGCCAGATCAGCAACTTTCCGGTGGTGGGCGCCGAGCTGTTG
CCCGTGCACTCCAAGAGCTTCTACAACGACCAGGCCGTCTACTCCCAACTCATCCGCCAGTTTACCTCTC
TGACCCACGTGTTCAATCGCTTTCCCGAGAACCAGATTTTGGCGCGCCCGCCAGCCCCCACCATCACCAC
CGTCAGTGAAAACGTTCCTGCTCTCACAGATCACGGGACGCTACCGCTGCGCAACAGCATCGGAGGAGTC
CAGCGAGTGACCATTACTGACGCCAGACGCCGCACCTGCCCCTACGTTTACAAGGCCCTGGGCATAGTCT
CGCCGCGCGTCCTATCGAGCCGCACTTTTTGAGCAAGCATGTCCATCCTTATATCGCCCAGCAATAACAC
AGGCTGGGGCCTGCGCTTCCCAAGCAAGATGTTTGGCGGGGCCAAGAAGCGCTCCGACCAACACCCAGTG
CGCGTGCGCGGGCACTACCGCGCGCCCTGGGGCGCGCACAAACGCGGCCGCACTGGGCGCACCACCGTCG
ATGACGCCATCGACGCGGTGGTGGAGGAGGCGCGCAACTACACGCCCACGCCGCCACCAGTGTCCACAGT
GGACGCGGCCATTCAGACCGTGGTGCGCGGAGCCCGGCGCTATGCTAAAATGAAGAGACGGCGGAGGCGC
GTAGCACGTCGCCACCGCCGCCGACCCGGCACTGCCGCCCAACGCGCGGCGGCGGCCCTGCTTAACCGCG
CACGTCGCACCGGCCGACGGGCGGCCATGCGGGCCGCTCGAAGGCTGGCCGCGGGTATTGTCACTGTGCC
CCCCAGGTCCAGGCGACGAGCGGCCGCCGCAGCAGCCGCGGCCATTAGTGCTATGACTCAGGGTCGCAGG
GGCAACGTGTATTGGGTGCGCGACTCGGTTAGCGGCCTGCGCGTGCCCGTGCGCACCCGCCCCCCGCGCA
ACTAGATTGCAAGAAAAAACTACTTAGACTCGTACTGTTGTATGTATCCAGCGGCGGCGGCGCGCAACGA
AGCTATGTCCAAGCGCAAAATCAAAGAAGAGATGCTCCAGGTCATCGCGCCGGAGATCTATGGCCCCCCG
AAGAAGGAAGAGCAGGATTACAAGCCCCGAAAGCTAAAGCGGGTCAAAAAGAAAAAGAAAGATGATGATG
ATGAACTTGACGACGAGGTGGAACTGCTGCACGCTACCGCGCCCAGGCGACGGGTACAGTGGAAAGGTCG
ACGCGTAAAACGTGTTTTGCGACCCGGCACCACCGTAGTCTTTACGCCCGGTGAGCGCTCCACCCGCACC
TACAAGCGCGTGTATGATGAGGTGTACGGCGACGAGGACCTGCTTGAGCAGGCCAACGAGCGCCTCGGGG
AGTTTGCCTACGGAAAGCGGCATAAGGACATGCTGGCGTTGCCGCTGGACGAGGGCAACCCAACACCTAG
CCTAAAGCCCGTAACACTGCAGCAGGTGCTGCCCGCGCTTGCACCGTCCGAAGAAAAGCGCGGCCTAAAG
CGCGAGTCTGGTGACTTGGCACCCACCGTGCAGCTGATGGTACCCAAGCGCCAGCGACTGGAAGATGTCT
TGGAAAAAATGACCGTGGAACCTGGGCTGGAGCCCGAGGTCCGCGTGCGGCCAATCAAGCAGGTGGCGCC
GGGACTGGGCGTGCAGACCGTGGACGTTCAGATACCCACTACCAGTAGCACCAGTATTGCCACCGCCACA
GAGGGCATGGAGACACAAACGTCCCCGGTTGCCTCAGCGGTGGCGGATGCCGCGGTGCAGGCGGTCGCTG
CGGCCGCGTCCAAGACCTCTACGGAGGTGCAAACGGACCCGTGGATGTTTCGCGTTTCAGCCCCCCGGCG
CCCGCGCGGTTCGAGGAAGTACGGCGCCGCCAGCGCGCTACTGCCCGAATATGCCCTACATCCTTCCATT
GCGCCTACCCCCGGCTATCGTGGCTACACCTACCGCCCCAGAAGACGAGCAACTACCCGACGCCGAACCA
CCACTGGAACCCGCCGCCGCCGTCGCCGTCGCCAGCCCGTGCTGGCCCCGATTTCCGTGCGCAGGGTGGC
TCGCGAAGGAGGCAGGACCCTGGTGCTGCCAACAGCGCGCTACCACCCCAGCATCGTTTAAAAGCCGGTC
TTTGTGGTTCTTGCAGATATGGCCCTCACCTGCCGCCTCCGTTTCCCGGTGCCGGGATTCCGAGGAAGAA
TGCACCGTAGGAGGGGCATGGCCGGCCACGGCCTGACGGGCGGCATGCGTCGTGCGCACCACCGGCGGCG
GCGCGCGTCGCACCGTCGCATGCGCGGCGGTATCCTGCCCCTCCTTATTCCACTGATCGCCGCGGCGATT
GGCGCCGTGCCCGGAATTGCATCCGTGGCCTTGCAGGCGCAGAGACACTGATTAAAAACAAGTTGCATGT
GGAAAAATCAAAATAAAAAGTCTGGACTCTCACGCTCGCTTGGTCCTGTAACTATTTTGTAGAATGGAAG
ACATCAACTTTGCGTCTCTGGCCCCGCGACACGGCTCGCGCCCGTTCATGGGAAACTGGCAAGATATCGG
CACCAGCAATATGAGCGGTGGCGCCTTCAGCTGGGGCTCGCTGTGGAGCGGCATTAAAAATTTCGGTTCC
ACCGTTAAGAACTATGGCAGCAAGGCCTGGAACAGCAGCACAGGCCAGATGCTGAGGGATAAGTTGAAAG
AGCAAAATTTCCAACAAAAGGTGGTAGATGGCCTGGCCTCTGGCATTAGCGGGGTGGTGGACCTGGCCAA
CCAGGCAGTGCAAAATAAGATTAACAGTAAGCTTGATCCCCGCCCTCCCGTAGAGGAGCCTCCACCGGCC
GTGGAGACAGTGTCTCCAGAGGGGCGTGGCGAAAAGCGTCCGCGCCCCGACAGGGAAGAAACTCTGGTGA
CGCAAATAGACGAGCCTCCCTCGTACGAGGAGGCACTAAAGCAAGGCCTGCCCACCACCCGTCCCATCGC
GCCCATGGCTACCGGAGTGCTGGGCCAGCACACACCCGTAACGCTGGACCTGCCTCCCCCCGCCGACACC
CAGCAGAAACCTGTGCTGCCAGGCCCGACCGCCGTTGTTGTAACCCGTCCTAGCCGCGCGTCCCTGCGCC
GCGCCGCCAGCGGTCCGCGATCGTTGCGGCCCGTAGCCAGTGGCAACTGGCAAAGCACACTGAACAGCAT
CGTGGGTCTGGGGGTGCAATCCCTGAAGCGCCGACGATGCTTCTGAATAGCTAACGTGTCGTATGTGTGT
CATGTATGCGTCCATGTCGCCGCCAGAGGAGCTGCTGAGCCGCCGCGCGCCCGCTTTCCAAGATGGCTAC
CCCTTCGATGATGCCGCAGTGGTCTTACATGCACATCTCGGGCCAGGACGCCTCGGAGTACCTGAGCCCC
GGGCTGGTGCAGTTTGCCCGCGCCACCGAGACGTACTTCAGCCTGAATAACAAGTTTAGAAACCCCACGG
TGGCGCCTACGCACGACGTGACCACAGACCGGTCCCAGCGTTTGACGCTGCGGTTCATCCCTGTGGACCG
TGAGGATACTGCGTACTCGTACAAGGCGCGGTTCACCCTAGCTGTGGGTGATAACCGTGTGCTGGACATG
GCTTCCACGTACTTTGACATCCGCGGCGTGCTGGACAGGGGCCCTACTTTTAAGCCCTACTCTGGCACTG
CCTACAACGCCCTGGCTCCCAAGGGTGCCCCAAATCCTTGCGAATGGGATGAAGCTGCTACTGCTCTTGA
AATAAACCTAGAAGAAGAGGACGATGACAACGAAGACGAAGTAGACGAGCAAGCTGAGCAGCAAAAAACT
CACGTATTTGGGCAGGCGCCTTATTCTGGTATAAATATTACAAAGGAGGGTATTCAAATAGGTGTCGAAG
GTCAAACACCTAAATATGCCGATAAAACATTTCAACCTGAACCTCAAATAGGAGAATCTCAGTGGTACGA
AACTGAAATTAATCATGCAGCTGGGAGAGTCCTTAAAAAGACTACCCCAATGAAACCATGTTACGGTTCA
TATGCAAAACCCACAAATGAAAATGGAGGGCAAGGCATTCTTGTAAAGCAACAAAATGGAAAGCTAGAAA
GTCAAGTGGAAATGCAATTTTTCTCAACTACTGAGGCGACCGCAGGCAATGGTGATAACTTGACTCCTAA
AGTGGTATTGTACAGTGAAGATGTAGATATAGAPACCCCAGACACTCATATTTCTTACATGCCCACTATT
AAGGAAGGTAACTCACGAGAACTAATGGGCCAACAATCTATGCCCAACAGGCCTAATTACATTGCTTTTA
GGGACAATTTTATTGGTCTAATGTATTACAACAGCACGGGTAATATGGGTGTTCTGGCGGGCCAAGCATC
GCAGTTGAATGCTGTTGTAGATTTGCAAGACAGAAACACAGAGCTTTCATACCAGCTTTTGCTTGATTCC
ATTGGTGATAGAACCAGGTACTTTTCTATGTGGAATCAGGCTGTTGACAGCTATGATCCAGATGTTAGAA
TTATTGAAAATCATGGAACTGAAGATGAACTTCCAAATTACTGCTTTCCACTGGGAGGTGTGATTAATAC
AGAGACTCTTACCAAGGTAAAACCTAAAACAGGTCAGGAAAATGGATGGGAAAAAGATGCTACAGAATTT
TCAGATAAAAATGAAATAAGAGTTGGAAATAATTTTGCCATGGAAATCAATCTAAATGCCAACCTGTGGA
GAAATTTCCTGTACTCCAACATAGCGCTGTATTTGCCCGACAAGCTAAAGTACAGTCCTTCCAACGTAAA
AATTTCTGATAACCCAAACACCTACGACTACATGAACAAGCGAGTGGTGGCTCCCGGGTTAGTGGACTGC
TACATTAACCTTGGAGCACGCTGGTCCCTTGACTATATGGACAACGTCAACCCATTTAACCACCACCGCA
ATGCTGGCCTGCGCTACCGCTCAATGTTGCTGGGCAATGGTCGCTATGTGCCCTTCCACATCCAGGTGCC
TCAGAAGTTCTTTGCCATTAAAAACCTCCTTCTCCTGCCGGGCTCATACACCTACGAGTGGAACTTCAGG
AAGGATGTTAACATGGTTCTGCAGAGCTCCCTAGGAAATGACCTAAGGGTTGACGGAGCCAGCATTAAGT
TTGATAGCATTTGCCTTTACGCCACCTTCTTCCCCATGGCCCACAACACCGCCTCCACGCTTGAGGCCAT
GCTTAGAAACGACACCAACGACCAGTCCTTTAACGACTATCTCTCCGCCGCCAACATGCTCTACCCTATA
CCCGCCAACGCTACCAACGTGCCCATATCCATCCCCTCCCGCAACTGGGCGGCTTTCCGCGGCTGGGCCT
TCACGCGCCTTAAGACTAAGGAAACCCCATCACTGGGCTCGGGCTACGACCCTTATTACACCTACTCTGG
CTCTATACCCTACCTAGATGGAACCTTTTACCTCAACCACACCTTTAAGAAGGTGGCCATTACCTTTGAC
TCTTCTGTCAGCTGGCCTGGCAATGACCGCCTGCTTACCCCCAACGAGTTTGAAATTAAGCGCTCAGTTG
ACGGGGAGGGTTACAACGTTGCCCAGTGTAACATGACCAAAGACTGGTTCCTGGTACAAATGCTAGCTAA
CTACAACATTGGCTACCAGGGCTTCTATATCCCAGAGAGCTACAAGGACCGCATGTACTCCTTCTTTAGA
AACTTCCAGCCCATGAGCCGTCAGGTGGTGGATGATACTAAATACAAGGACTACCAACAGGTGGGCATCC
TACACCAACACAACAACTCTGGATTTGTTGGCTACCTTGCCCCCACCATGCGCGAAGGACAGGCCTACCC
TGCTAACTTCCCCTATCCGCTTATAGGCAAGACCGCAGTTGACAGCATTACCCAGAAAAAGTTTCTTTGC
GATCGCACCCTTTGGCGCATCCCATTCTCCAGTAACTTTATGTCCATGGGCGCACTCACAGACCTGGGCC
AAAACCTTCTCTACGCCAACTCCGCCCACGCGCTAGACATGACTTTTGAGGTGGATCCCATGGACGAGCC
CACCCTTCTTTATGTTTTGTTTGAAGTCTTTGACGTGGTCCGTGTGCACCGGCCGCACCGCGGCGTCATC
GAAACCGTGTACCTGCGCACGCCCTTCTCGGCCGGCAACGCCACAACATAAAGAAGCAAGCAACATCAAC
AACAGCTGCCGCCATGGGCTCCAGTGAGCAGGAACTGAAAGCCATTGTCAAAGATCTTGGTTGTGGGCCA
TATTTTTTGGGCACCTATGACAAGCGCTTTCCAGGCTTTGTTTCTCCACACAAGCTCGCCTGCGCCATAG
TCAATACGGCCGGTCGCGAGACTGGGGGCGTACACTGGATGGCCTTTGCCTGGAACCCGCACTCAAAAAC
ATGCTACCTCTTTGAGCCCTTTGGCTTTTCTGACCAGCGACTCAAGCAGGTTTACCAGTTTGAGTACGAG
TCACTCCTGCGCCGTAGCGCCATTGCTTCTTCCCCCGACCGCTGTATAACGCTGGAAAAGTCCACCCAAA
GCGTACAGGGGCCCAACTCGGCCGCCTGTGGACTATTCTGCTGCATGTTTCTCCACGCCTTTGCCAACTG
GCCCCAAACTCCCATGGATCACAACCCCACCATGAACCTTATTACCGGGGTACCCAACTCCATGCTCAAC
AGTCCCCAGGTACAGCCCACCCTGCGTCGCAACCAGGAACAGCTCTACAGCTTCCTGGAGCGCCACTCGC
CCTACTTCCGCAGCCACAGTGCGCAGATTAGGAGCGCCACTTCTTTTTGTCACTTGAAAAACATGTAAAA
ATAATGTACTAGAGACACTTTCAATAAAGGCAAATGCTTTTATTTGTACACTCTCGGGTGATTATTTACC
CCCACCCTTGCCGTCTGCGCCGTTTAAAAATCAAAGGGGTTCTGCCGCGCATCGCTATGCGCCACTGGCA
GGGACACGTTGCGATACTGGTGTTTAGTGCTCCACTTAAACTCAGGCACAACCATCCGCGGCAGCTCGGT
GAAGTTTTCACTCCACAGGCTGCGCACCATCACCAACGCGTTTAGCAGGTCGGGCGCCGATATCTTGAAG
TCGCAGTTGGGGCCTCCGCCCTGCGCGCGCGAGTTGCGATACACAGGGTTGCAGCACTGGAACACTATCA
GCGCCGGGTGGTGCACGCTGGCCAGCACGCTCTTGTCGGAGATCAGATCCGCGTCCAGGTCCTCCGCGTT
GCTCAGGGCGAACGGAGTCAACTTTGGTAGCTGCCTTCCCAAAAAGGGCGCGTGCCCAGGCTTTGAGTTG
CACTCGCACCGTAGTGGCATCAAAAGGTGACCGTGCCCGGTCTGGGCGTTAGGATACAGCGCCTGCATAA
AAGCCTTGATCTGCTTAAAAGCCACCTGAGCCTTTGCGCCTTCAGAGAAGAACATGCCGCAAGACTTGCC
GGAAAACTGATTGGCCGGACAGGCCGCGTCGTGCACGCAGCACCTTGCGTCGGTGTTGGAGATCTGCACC
ACATTTCGGCCCCACCGGTTCTTCACGATCTTGGCCTTGCTAGACTGCTCCTTCAGCGCGCGCTGCCCGT
TTTCGCTCGTCACATCCATTTCAATCACGTGCTCCTTATTTATCATAATGCTTCCGTGTAGACACTTAAG
CTCGCCTTCGATCTCAGCGCAGCGGTGCAGCCACAACGCGCAGCCCGTGGGCTCGTGATGCTTGTAGGTC
ACCTCTGCAAACGACTGCAGGTACGCCTGCAGGAATCGCCCCATCATCGTCACAAAGGTCTTGTTGCTGG
TGAAGGTCAGCTGCAACCCGCGGTGCTCCTCGTTCAGCCAGGTCTTGCATACGGCCGCCAGAGCTTCCAC
TTGGTCAGGCAGTAGTTTGAAGTTCGCCTTTAGATCGTTATCCACGTGGTACTTGTCCATCAGCGCGCGC
GCAGCCTCCATGCCCTTCTCCCACGCAGACACGATCGGCACACTCAGCGGGTTCATCACCGTAATTTCAC
TTTCCGCTTCGCTGGGCTCTTCCTCTTCCTCTTGCGTCCGCATACCACGCGCCACTGGGTCGTCTTCATT
CAGCCGCCGCACTGTGCGCTTACCTCCTTTGCCATGCTTGATTAGCACCGGTGGGTTGCTGAAACCCACC
ATTTGTAGCGCCACATCTTCTCTTTCTTCCTCGCTGTCCACGATTACCTCTGGTGATGGCGGGCGCTCGG
GCTTGGGAGAAGGGCGCTTCTTTTTCTTCTTGGGCGCAATGGCCAAATCCGCCGCCGAGGTCGATGGCCG
CGGGCTGGGTGTGCGCGGCACCAGCGCGTCTTGTGATGAGTCTTCCTCGTCCTCGGACTCGATACGCCGC
CTCATCCGCTTTTTTGGGGGCGCCCGGGGAGGCGGCGGCGACGGGGACGGGGACGACACGTCCTCCATGG
TTGGGGGACGTCGCGCCGCACCGCGTCCGCGCTCGGGGGTGGTTTCGCGCTGCTCCTCTTCCCGACTGGC
CATTTCCTTCTCCTATAGGCAGAAAAAGATCATGGAGTCAGTCGAGAAGAAGGACAGCCTAACCGCCCCC
TCTGAGTTCGCCACCACCGCCTCCACCGATGCCGCCAACGCGCCTACCACCTTCCCCGTCGAGGCACCCC
CGCTTGAGGAGGAGGAAGTGATTATCGAGCAGGACCCAGGTTTTGTAAGCGAAGACGACGAGGACCGCTC
AGTACCAACAGAGGATAAAAAGCAAGACCAGGACAACGCAGAGGCAAACGAGGAACAAGTCGGGCGGGGG
GACGAAAGGCATGGCGACTACCTAGATGTGGGAGACGACGTGCTGTTGAAGCATCTGCAGCGCCAGTGCG
CCATTATCTGCGACGCGTTGCAAGAGCGCAGCGATGTGCCCCTCGCCATAGCGGATGTCAGCCTTGCCTA
CGAACGCCACCTATTCTCACCGCGCGTACCCCCCAAACGCCAAGAAAACGGCACATGCGAGCCCAACCCG
CGCCTCAACTTCTACCCCGTATTTGCCGTGCCAGAGGTGCTTGCCACCTATCACATCTTTTTCCAAAACT
GCAAGATACCCCTATCCTGCCGTGCCAACCGCAGCCGAGCGGACAAGCAGCTGGCCTTGCGGCAGGGCGC
TGTCATACCTGATATCGCCTCGCTCAACGAAGTGCCAAAAATCTTTGAGGGTCTTGGACGCGACGAGAAG
CGCGCGGCAAACGCTCTGCAACAGGAAAACAGCGAAAATGAAAGTCACTCTGGAGTGTTGGTGGAACTCG
AGGGTGACAACGCGCGCCTAGCCGTACTAAAACGCAGCATCGAGGTCACCCACTTTGCCTACCCGGCACT
TAACCTACCCCCCAAGGTCATGAGCACAGTCATGAGTGAGCTGATCGTGCGCCGTGCGCAGCCCCTGGAG
AGGGATGCAAATTTGCAAGAACAAACAGAGGAGGGCCTACCCGCAGTTGGCGACGAGCAGCTAGCGCGCT
GGCTTCAAACGCGCGAGCCTGCCGACTTGGAGGAGCGACGCAAACTAATGATGGCCGCAGTGCTCGTTAC
CGTGGAGCTTGAGTGCATGCAGCGGTTCTTTGCTGACCCGGAGATGCAGCGCAAGCTAGAGGAAACATTG
CACTACACCTTTCGACAGGGCTACGTACGCCAGGCCTGCAAGATCTCCAACGTGGAGCTCTGCAACCTGG
TCTCCTACCTTGGAATTTTGCACGAAAACCGCCTTGGGCAAAACGTGCTTCATTCCACGCTCAAGGGCGA
GGCGCGCCGCGACTACGTCCGCGACTGCGTTTACTTATTTCTATGCTACACCTGGCAGACGGCCATGGGC
GTTTGGCAGCAGTGCTTGGAGGAGTGCAACCTCAAGGAGCTGCAGAAACTGCTAAAGCAAAACTTGAAGG
ACCTATGGACGGCCTTCAACGAGCGCTCCGTGGCCGCGCACCTGGCGGACATCATTTTCCCCGAACGCCT
GCTTAAAACCCTGCAACAGGGTCTGCCAGACTTCACCAGTCAAAGCATGTTGCAGAACTTTAGGAACTTT
ATCCTAGAGCGCTCAGGAATCTTGCCCGCCACCTGCTGTGCACTTCCTAGCGACTTTGTGCCCATTAAGT
ACCGCGAATGCCCTCCGCCGCTTTGGGGCCACTGCTACCTTCTGCAGCTAGCCAACTACCTTGCCTACCA
CTCTGACATAATGGAAGACGTGAGCGGTGACGGTCTACTGGAGTGTCACTGTCGCTGCAACCTATGCACC
CCGCACCGCTCCCTGGTTTGCAATTCGCAGCTGCTTAACGAAAGTCAAATTATCGGTACCTTTGAGCTGC
AGGGTCCCTCGCCTGACGAAAAGTCCGCGGCTCCGGGGTTGAAACTCACTCCGGGGCTGTGGACGTCGGC
TTACCTTCGCAAATTTGTACCTGAGGACTACCACGCCCACGAGATTAGGTTCTACGAAGACCAATCCCGC
CCGCCAAATGCGGAGCTTACCGCCTGCGTCATTACCCAGGGCCACATTCTTGGCCAATTGCAAGCCATCA
ACAAAGCCCGCCAAGAGTTTCTGCTACGAAAGGGACGGGGGGTTTACTTGGACCCCCAGTCCGGCGAGGA
GCTCAACCCAATCCCCCCGCCGCCGCAGCCCTATCAGCAGCAGCCGCGGGCCCTTGCTTCCCAGGATGGC
ACCCAAAAAGAAGCTGCAGCTGCCGCCGCCACCCACGGACGAGGAGGAATACTGGGACAGTCAGGCAGAG
GAGGTTTTGGACGAGGAGGAGGAGGACATGATGGAAGACTGGGAGAGCCTAGACGAGGAAGCTTCCGAGG
TCGAAGAGGTGTCAGACGAAACACCGTCACCCTCGGTCGCATTCCCCTCGCCGGCGCCCCAGAAATCGGC
AACCGGTTCCAGCATGGCTACAACCTCCGCTCCTCAGGCGCCGCCGGCACTGCCCGTTCGCCGACCCAAC
CGTAGATGGGACACCACTGGAACCAGGGCCGGTAAGTCCAAGCAGCCGCCGCCGTTAGCCCAAGAGCAAC
AACAGCGCCAAGGCTACCGCTCATGGCGCGGGCACAAGAACGCCATAGTTGCTTGCTTGCAAGACTGTGG
GGGCAACATCTCCTTCGCCCGCCGCTTTCTTCTCTACCATCACGGCGTGGCCTTCCCCCGTAACATCCTG
CATTACTACCGTCATCTCTACAGCCCATACTGCACCGGCGGCAGCGGCAGCGGCAGCAACAGCAGCGGCC
ACACAGAAGCAAAGGCGACCGGATAGCAAGACTCTGACAAAGCCCAAGAAATCCACAGCGGCGGCAGCAG
CAGGAGGAGGAGCGCTGCGTCTGGCGCCCAACGAACCCGTATCGACCCGCGAGCTTAGAAACAGGATTTT
TCCCACTCTGTATGCTATATTTCAACAGAGCAGGGGCCAAGAACAAGAGCTGAAAATAAAAAACAGGTCT
CTGCGATCCCTCACCCGCAGCTGCCTGTATCACAAAAGCGAAGATCAGCTTCGGCGCACGCTGGAAGACG
CGGAGGCTCTCTTCAGTAAATACTGCGCGCTGACTCTTAAGGACTAGTTTCGCGCCCTTTCTCAAATTTA
AGCGCGAAAACTACGTCATCTCCAGCGGCCACACCCGGCGCCAGCACCTGTCGTCAGCGCCATTATGAGC
AAGGAAATTCCCACGCCCTACATGTGGAGTTACCAGCCACAAATGGGACTTGCGGCTGGAGCTGCCCAAG
ACTACTCAACCCGAATAAACTACATGAGCGCGGGACCCCACATGATATCCCGGGTCAACGGAATCCGCGC
CCACCGAAACCGAATTCTCTTGGAACAGGCGGCTATTACCACCACACCTCGTAATAACCTTAATCCCCGT
AGTTGGCCCGCTGCCCTGGTGTACCAGGAAAGTCCCGCTCCCACCACTGTGGTACTTCCCAGAGACGCCC
AGGCCGAAGTTCAGATGACTAACTCAGGGGCGCAGCTTGCGGGCGGCTTTCGTCACAGGGTGCGGTCGCC
CGGGCAGGGTATAACTCACCTGACAATCAGAGGGCGAGGTATTCAGCTCAACGACGAGTCGGTGAGCTCC
TCGCTTGGTCTCCGTCCGGACGGGACATTTCAGATCGGCGGCGCCGGCCGTCCTTCATTCACGCCTCGTC
AGGCAATCCTAACTCTGCAGACCTCGTCCTCTGAGCCGCGCTCTGGAGGCATTGGAACTCTGCAATTTAT
TGAGGAGTTTGTGCCATCGGTCTACTTTAACCCCTTCTCGGGACCTCCCGGCCACTATCCGGATCAATTT
ATTCCTAACTTTGACGCGGTAAAGGACTCGGCGGACGGCTACGACTGAATGTTAAGTGGAGAGGCAGAGC
AACTGCGCCTGAAACACCTGGTCCACTGTCGCCGCCACAAGTGCTTTGCCCGCGACTCCGGTGAGTTTTG
CTACTTTGAATTGCCCGAGGATCATATCGAGGGCCCGGCGCACGGCGTCCGGCTTACCGCCCAGGGAGAG
CTTGCCCGTAGCCTGATTCGGGAGTTTACCCAGCGCCCCCTGCTAGTTGAGCGGGACAGGGGACCCTGTG
TTCTCACTGTGATTTGCAACTGTCCTAACCTTGGATTACATCAAGATCTTTGTTGCCATCTCTGTGCTGA
GTATAATAAATACAGAAATTAAAATATACTGGGGCTCCTATCGCCATCCTGTAAACGCCACCGTCTTCAC
CCGCCCAAGCAAACCAAGGCGAACCTTACCTGGTACTTTTAACATCTCTCCCTCTGTGATTTACAACAGT
TTCAACCCAGACGGAGTGAGTCTACGAGAGAACCTCTCCGAGCTCAGCTACTCCATCAGAAAAAACACCA
CCCTCCTTACCTGCCGGGAACGTACGAGTGCGTCACCGGCCGCTGCACCACACCTACCGCCTGACCGTAA
ACCAGACTTTTTCCGGACAGACCTCAATAACTCTGTTTACCAGAACAGGAGGTGAGCTTAGAAAACCCTT
AGGGTATTAGGCCAAAGGCGCAGCTACTGTGGGGTTTATGAACAATTCAAGCAACTCTACGGGCTATTCT
AATTCAGGTTTCTCTAGAAATGGACGGAATTATTACAGAGCAGCGCCTGCTAGAAAGACGCAGGGCAGCG
GCCGAGCAACAGCGCATGAATCAAGAGCTCCAAGACATGGTTAACTTGCACCAGTGCAAAAGGGGTATCT
TTTGTCTGGTAAAGCAGGCCAAAGTCACCTACGACAGTAATACCACCGGACACCGCCTTAGCTACAAGTT
GCCAACCAAGCGTCAGAPATTGGTGGTCATGGTGGGAGAAAAGCCCATTACCATAACTCAGCACTCGGTA
GAAACCGAAGGCTGCATTCACTCACCTTGTCAAGGACCTGAGGATCTCTGCACCCTTATTAAGACCCTGT
GCGGTCTCAAAGATCTTATTCCCTTTAACTAATAAAAAAAAATAATAAAGCATCACTTACTTAAAATCAG
TTAGCAAATTTCTGTCCAGTTTATTCAGCAGCACCTCCTTGCCCTCCTCCCAGCTCTGGTATTGCAGCTT
CCTCCTGGCTGCAAACTTTCTCCACAATCTAAATGGAATGTCAGTTTCCTCCTGTTCCTGTCCATCCGCA
CCCACTATCTTCATGTTGTTGCAGATGAAGCGCGCAAGACCGTCTGAAGATACCTTCAACCCCGTGTATC
CATATGACACGGAAACCGGTCCTCCAACTGTGCCTTTTCTTACTCCTCCCTTTGTATCCCCCAATGGGTT
TCAAGAGAGTCCCCCTGGGGTACTCTCTTTGCGCCTATCCGAACCTCTAGTTACCTCCAATGGCATGCTT
GCGCTCAAAATGGGCAACGGCCTCTCTCTGGACGAGGCCGGCAACCTTACCTCCCAAAATGTAACCACTG
TGAGCCCACCTCTCAAAAAAACCAAGTCAAACATAAACCTGGAAATATCTGCACCCCTCACAGTTACCTC
AGAAGCCCTAACTGTGGCTGCCGCCGCACCTCTAATGGTCGCGGGCAACACACTCACCATGCAATCACAG
GCCCCGCTAACCGTGCACGACTCCAAACTTAGCATTGCCACCCAAGGACCCCTCACAGTGTCAGAAGGAA
AGCTAGCCCTGCAAACATCAGGCCCCCTCACCACCACCGATAGCAGTACCCTTACTATCACTGCCTCACC
CCCTCTAACTACTGCCACTGGTAGCTTGGGCATTGACTTGAAAGAGCCCATTTATACACAAAATGGAAAA
CTAGGACTAAAGTACGGGGCTCCTTTGCATGTAACAGACGACCTAAACACTTTGACCGTAGCAACTGGTC
CAGGTGTGACTATTAATAATACTTCCTTGCAAACTAAAGTTACTGGAGCCTTGGGTTTTGATTCACAAGG
CAATATGCAACTTAATGTAGCAGGAGGACTAAGGATTGATTCTCAAAACAGACGCCTTATACTTGATGTT
AGTTATCCGTTTGATGCTCAAAACCAACTAAATCTAAGACTAGGACAGGGCCCTCTTTTTATAAACTCAG
CCCACAACTTGGATATTAACTACAACAAAGGCCTTTACTTGTTTACAGCTTCAAACAATTCCAAAAAGCT
TGAGGTTAACCTAAGCACTGCCAAGGGGTTGATGTTTGACGCTACAGCCATAGCCATTAATGCAGGAGAT
GGGCTTGAATTTGGTTCACCTAATGCACCAAACACAAATCCCCTCAAAACAAAAATTGGCCATGGCCTAG
AATTTGATTCAAACAAGGCTATGGTTCCTAAACTAGGAACTGGCCTTAGTTTTGACAGCACAGGTGCCAT
TACAGTAGGAAACAAAAATAATGATAAGCTAACTTTGTGGACCACACCAGCTCCATCTCCTAACTGTAGA
CTAAATGCAGAGAAAGATGCTAAACTCACTTTGGTCTTAACAAAATGTGGCAGTCAAATACTTGCTACAG
TTTCAGTTTTGGCTGTTAAAGGCAGTTTGGCTCCAATATCTGGAACAGTTCAAAGTGCTCATCTTATTAT
AAGATTTGACGAAAATGGAGTGCTACTAAACAATTCCTTCCTGGACCCAGAATATTGGAACTTTAGAAAT
GGAGATCTTACTGAAGGCACAGCCTATACAAACGCTGTTGGATTTATGCCTAACCTATCAGCTTATCCAA
AATCTCACGGTAAAACTGCCAAAAGTAACATTGTCAGTCAAGTTTACTTAAACGGAGACAAAACTAAACC
TGTAACACTAACCATTACACTAAACGGTACACAGGAAACAGGAGACACAACTCCAAGTGCATACTCTATG
TCATTTTCATGGGACTGGTCTGGCCACAACTACATTAATGAAATATTTGCCACATCCTCTTACACTTTTT
CATACATTGCCCAAGAATAAAGAATCGTTTGTGTTATGTTTCAACGTGTTTATTTTTCAATTGCAGAAAA
TTTCGAATCATTTTTCATTCAGTAGTATAGCCCCACCACCACATAGCTTATACAGATCACCGTACCTTAA
TCAAACTCACAGAACCCTAGTATTCAACCTGCCACCTCCCTCCCAACACACAGAGTACACAGTCCTTTCT
CCCCGGCTGGCCTTAAAAAGCATCATATCATGGGTAACAGACATATTCTTAGGTGTTATATTCCACACGG
TTTCCTGTCGAGCCAAACGCTCATCAGTGATATTAATAAACTCCCCGGGCAGCTCACTTAAGTTCATGTC
GCTGTCCAGCTGCTGAGCCACAGGCTGCTGTCCAACTTGCGGTTGCTTAACGGGCGGCGAAGGAGAAGTC
CACGCCTACATGGGGGTAGAGTCATAATCGTGCATCAGGATAGGGCGGTGGTGCTGCAGCAGCGCGCGAA
TAAACTGCTGCCGCCGCCGCTCCGTCCTGCAGGAATACAACATGGCAGTGGTCTCCTCAGCGATGATTCG
CACCGCCCGCAGCATAAGGCGCCTTGTCCTCCGGGCACAGCAGCGCACCCTGATCTCACTTAAATCAGCA
CAGTAACTGCAGCACAGCACCACAATATTGTTCAAAATCCCACAGTGCAAGGCGCTGTATCCAAAGCTCA
TGGCGGGGACCACAGAACCCACGTGGCCATCATACCACAAGCGCAGGTAGATTAAGTGGCGACCCCTCAT
AAACACGCTGGACATAAACATTACCTCTTTTGGCATGTTGTAATTCACCACCTCCCGGTACCATATAAAC
CTCTGATTAAACATGGCGCCATCCACCACCATCCTAAACCAGCTGGCCAAAACCTGCCCGCCGGCTATAC
ACTGCAGGGAACCGGGACTGGAACAATGACAGTGGAGAGCCCAGGACTCGTAACCATGGATCATCATGCT
CGTCATGATATCAATGTTGGCACAACACAGGCACACGTGCATACACTTCCTCAGGATTACAAGCTCCTCC
CGCGTTAGAACCATATCCCAGGGAACAACCCATTCCTGAATCAGCGTAAATCCCACACTGCAGGGAAGAC
CTCGCACGTAACTCACGTTGTGCATTGTCAAAGTGTTACATTCGGGCAGCAGCGGATGATCCTCCAGTAT
GGTAGCGCGGGTTTCTGTCTCAAAAGGAGGTAGACGATCCCTACTGTACGGAGTGCGCCGAGACAACCGA
GATCGTGTTGGTCGTAGTGTCATGCCAATGGAAACGCCGGACGTAGTCATATTTCCTGAAGCAAAACCAG
GTGCGGGCGTGACAAACAGATCTGCGTCTCCGGTCTCGCCGCTTAGATCGCTCTGTGTAGTAGTTGTAGT
ATATCCACTCTCTCAAAGCATCCAGGCGCCCCCTGGCTTCGGGTTCTATGTAAACTCCTTCATGCGCCGC
TGCCCTGATAACATCCACCACCGCAGAATAAGCCACACCCAGCCAACCTACACATTCGTTCTGCGAGTCA
CACACGGGAGGAGCGGGAAGAGCTGGAAGAACCATGTTTTTTTTTTTATTCCAAAAGATTATCCAAAACC
TCAAAATGAAGATCTATTAAGTGAACGCGCTCCCCTCCGGTGGCGTGGTCAAACTCTACAGCCAAAGAAC
AGATAATGGCATTTGTAAGATGTTGCACAATGGCTTCCAAAAGGCAAACGGCCCTCACGTCCAAGTGGAC
GTAAAGGCTAAACCCTTCAGGGTGAATCTCCTCTATAAACATTCCAGCACCTTCAACCATGCCCAAATAA
TTCTCATCTCGCCACCTTCTCAATATATCTCTAAGCAAATCCCGAATATTAAGTCCGGCCATTGTAAAAA
TCTGCTCCAGAGCGCCCTCCACCTTCAGCCTCAAGCAGCGAATCATGATTGCAAAAATTCAGGTTCCTCA
CAGACCTGTATAAGATTCAAAAGCGGAACATTAACAAAAATACCGCGATCCCGTAGGTCCCTTCGCAGGG
CCAGCTGAACATAATCGTGCAGGTCTGCACGGACCAGCGCGGCCACTTCCCCGCCAGGAACCTTGACAAA
AGAACCCACACTGATTATGACACGCATACTCGGAGCTATGCTAACCAGCGTAGCCCCGATGTAAGCTTTG
TTGCATGGGCGGCGATATAAAATGCAAGGTGCTGCTCAAAAAATCAGGCAAAGCCTCGCGCAAAAAAGAA
AGCACATCGTAGTCATGCTCATGCAGATAAAGGCAGGTAAGCTCCGGAACCACCACAGAAAAAGACACCA
TTTTTCTCTCAAACATGTCTGCGGGTTTCTGCATAAACACAAAATAAAATAACAAAAAAACATTTAAACA
TTAGAAGCCTGTCTTACAACAGGAAAAACAACCCTTATAAGCATAAGACGGACTACGGCCATGCCGGCGT
GACCGTAAAAAAACTGGTCACCGTGATTAAAAAGCACCACCGACAGCTCCTCGGTCATGTCCGGAGTCAT
AATGTAAGACTCGGTAAACACATCAGGTTGATTCACATCGGTCAGTGCTAAAAAGCGACCGAAATAGCCC
GGGGGAATACATACCCGCAGGCGTAGAGACAACATTACAGCCCCCATAGGAGGTATAACAAAATTAATAG
GAGAGAAAAACACATAAACACCTGAAAAACCCTCCTGCCTAGGCAAAATAGCACCCTCCCGCTCCAGAAC
AACATACAGCGCTTCCACAGCGGCAGCCATAACAGTCAGCCTTACCAGTAAAAAAGAAAACCTATTAAAA
AAACACCACTCGACACGGCACCAGCTCAATCAGTCACAGTGTAAAAAAGGGCCAAGTGCAGAGCGAGTAT
ATATAGGACTAAAAAATGACGTAACGGTTAAAGTCCACAAAAAACACCCAGAAAACCGCACGCGAACCTA
CGCCCAGAAACGAAAGCCAAAAAACCCACAACTTCCTCAAATCGTCACTTCCGTTTTCCCACGTTACGTC
ACTTCCCATTTTAAGAAAACTACAATTCCCAACACATACAAGTTACTCCGCCCTAAAACCTACGTCACCC
GCCCCGTTCCCACGCCCCGCGCCACGTCACAAACTCCACCCCCTCATTATCATATTGGCTTCAATCCAAA
ATAAGGTATATTATTGATGATGTTAATTAATTTAAATCCGCATGCGATATCGAGCTCTCCCGGGAATTCG
GATCTGCGACGCGAGGCTGGATGGCCTTCCCCATTATGATTCTTCTCGCTTCCGGCGGCATCGGGATGCC
CGCGTTGCAGGCCATGCTGTCCAGGCAGGTAGATGACGACCATCAGGGACAGCTTCACGGCCAGCAAAAG
GCCAGGAACCGTAAAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCGCCTTTCTCCCTTCG
GGAAGCGTGGCGCTTTCTCAATGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATGA
GATTATCAAAAAGGATCTTCACCTAGATCCTTTTAAATCAATCTAAAGTATATATGAGTAAACTTGGTCT
GACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGNTGCAGGCATCGTGGTGTCACGCTCGTCGTT
TGGTATGGCTTCATTCAGCTCCGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAACACGGGAT
AATACCGCGCCACATAGCAGAACTTTAAAAGTGCTCATCATTGGAAAACGTTCTTCGGGGCGAAAACTCT
CAAGGATCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGTTATT
GTCTCATGAGCGGATACATATTTGAATGTATTTAGAAAAATAAACAAATAGGGGTTCCGCGCACATTTCC
CCGAAAAGTGCCACCTGACGTCTAAGAAACCATTATTATCATGACATTAACCTATAAAAATAGGCGTATC
ACGAGGCCCTTTCGTCTTCAAGGATCCGAATTCCCGGGAGAGCTCGATATCGCATGCGGATTTAAATTAA
TTAA

TABLE 11
Nucleotide sequence of pAd/CMV/V5-GW/lacZ.PL-DEST ™.

CATCATCAATAATATACCTTATTTTGGATTGAAGCCAATATGATAATGAGGGGGTGGAGTTTGTGACGTG
GCGCGGGGCGTGGGAACGGGGCGGGTGACGTAGTAGTGTGGCGGAAGTGTGATGTTGCAAGTGTGGCGGA
ACACATGTAAGCGACGGATGTGGCAAAAGTGACGTTTTTGGTGTGCGCCGGTGTACACAGGAAGTGACAA
TTTTCGCGCGGTTTTAGGCGGATGTTGTAGTAAATTTGGGCGTAACCGAGTAAGATTTGGCCATTTTCGC
GGGAAAACTGAATAAGAGGAAGTGAAATCTGAATAATTTTGTGTTACTCATAGCGCGTAATATTTGTCTA
GGGCCGCGGGGACTTTGACCGTTTACGTGGAGACTCGCCCAGGTGTTTTTCTCAGGTGTTTTCCGCGTTC
CGGGTCAAAGTTGGCGTTTTATTATTATAGTCAGTCGAAGCTTGGATCCGGTACCTCTAGAATTCTCGAG
CGGCCGCTAGCGACATCGGATCTCCCGATCCCCTATGGTCGACTCTCAGTACAATCTGCTCTGATGCCGC
ATAGTTAAGCCAGTATCTGCTCCCTGCTTGTGTGTTGGAGGTCGCTGAGTAGTGCGCGAGCAAAATTTAA
GCTACAACAAGGCAAGGCTTGACCGACAATTGCATGAAGAATCTGCTTAGGGTTAGGCGTTTTGCGCTGC
TTCGCGATGTACGGGCCAGATATACGCGTTGACATTGATTATTGACTAGTTATTAATAGTAATCAATTAC
GGGGTCATTAGTTCATAGCCCATATATGGAGTTCCGCGTTACATAACTTACGGTAAATGGCCCGCCTGGC
TGACCGCCCAACGACCCCCGCCCATTGACGTCAATAATGACGTATGTTCCCATAGTAACGCCAATAGGGA
CTTTCCATTGACGTCAATGGGTGGACTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTGATGCGGT
TTTGGCAGTACATCAATGGGCGTGGATAGCGGTTTGACTCACGGGGATTTCCAAGTCTCCACCCCATTGA
CGTCAATGGGAGTTTGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGTTAAG
CTATCAACAAGTTTGTACAAAAAAGCAGGCTCCGCGGCCGCCCCCTTCACCATGATAGATCCCGTCGTTT
TACAACGTCGTGACTGGGAAAACCCTGGCGTTACCCAACTTAATCGCCTTGCAGCACATCCCCCTTTCGC
CAGCTGGCGTAATAGCGAAGAGGCCCGCACCGATCGCCCTTCCCAACAGTTGCGCAGCCTGAATGGCGAA
TGGCGCTTTGCCTGGTTTCCGGCACCAGAAGCGGTGCCGGAAAGCTGGCTGGAGTGCGATCTTCCTGAGG
CCGATACTGTCGTCGTCCCCTCAAACTGGCAGATGCACGGTTACGATGCGCCCATCTACACCAACGTAAC
CTATCCCATTACGGTCAATCCGCCGTTTGTTCCCACGGAGAATCCGACGGGTTGTTACTCGCTCACATTT
AATGTTGATGAAAGCTGGCTACAGGAAGGCCAGACGCGAATTATTTTTGATGGCGTTAACTCGGCGTTTC
ATCTGTGGTGCAACGGGCGCTGGGTCGGTTACGGCCAGGACAGTCGTTTGCCGTCTGAATTTGACCTGAG
CGCATTTTTACGCGCCGGAGAAAACCGCCTCGCGGTGATGGTGCTGCGTTGGAGTGACGGCAGTTATCTG
GAAGATCAGGATATGTGGCGGATGAGCGGCATTTTCCGTGACGTCTCGTTGCTGCATAAACCGACTACAC
AAATCAGCGATTTCCATGTTGCCACTCGCTTTAATGATGATTTCAGCCGCGCTGTACTGGAGGCTGAAGT
TCAGATGTGCGGCGAGTTGCGTGACTACCTACGGGTAACAGTTTCTTTATGGCAGGGTGAAACGCAGGTC
GCCAGCGGCACCGCGCCTTTCGGCGGTGAAATTATCGATGAGCGTGGTGGTTATGCCGATCGCGTCACAC
TACGTCTGAACGTCGAAAACCCGAAACTGTGGAGCGCCGAAATCCCGAATCTCTATCGTGCGGTGGTTGA
ACTGCACACCGCCGACGGCACGCTGATTGAAGCAGAAGCCTGCGATGTCGGTTTCCGCGAGGTGCGGATT
GAAAATGGTCTGCTGCTGCTGAACGGCAAGCCGTTGCTGATTCGAGGCGTTAACCGTCACGAGCATCATC
CTCTGCATGGTCAGGTCATGGATGAGCAGACGATGGTGCAGGATATCCTGCTGATGAAGCAGAACAACTT
TAACGCCGTGCGCTGTTCGCATTATCCGAACCATCCGCTGTGGTACACGCTGTGCGACCGCTACGGCCTG
TATGTGGTGGATGAAGCCAATATTGAAACCCACGGCATGGTGCCAATGAATCGTCTGACCGATGATCCGC
GCTGGCTACCGGCGATGAGCGAACGCGTAACGCGAATGGTGCAGCGCGATCGTAATCACCCGAGTGTGAT
CATCTGGTCGCTGGGGAATGAATCAGGCCACGGCGCTAATCACGACGCGCTGTATCGCTGGATCAAATCT
GTCGATCCTTCCCGCCCGGTGCAGTATGAAGGCGGCGGAGCCGACACCACGGCCACCGATATTATTTGCC
CGATGTACGCGCGCGTGGATGAAGACCAGCCCTTCCCGGCTGTGCCGAAATGGTCCATCAAAAAATGGCT
TTCGCTACCTGGAGAGACGCGCCCGCTGATCCTTTGCGAATACGCCCACGCGATGGGTAACAGTCTTGGC
GGTTTCGCTAAATACTGGCAGGCGTTTCGTCAGTATCCCCGTTTACAGGGCGGCTTCGTCTGGGACTGGG
TGGATCAGTCGCTGATTAAATATGATGAAAACGGCAACCCGTGGTCGGCTTACGGCGGTGATTTTGGCGA
TACGCCGAACGATCGCCAGTTCTGTATGAACGGTCTGGTCTTTGCCGACCGCACGCCGCATCCAGCGCTG
ACGGAAGCAAAACACCAGCAGCAGTTTTTCCAGTTCCGTTTATCCGGGCAAACCATCGAAGTGACCAGCG
AATACCTGTTCCGTCATAGCGATAACGAGCTCCTGCACTGGATGGTGGCGCTGGATGGTAAGCCGCTGGC
AAGCGGTGAAGTGCCTCTGGATGTCGCTCCACAAGGTAAACAGTTGATTGAACTGCCTGAACTACCGCAG
CCGGAGAGCGCCGGGCAACTCTGGCTCACAGTACGCGTAGTGCAACCGAACGCGACCGCATGGTCAGAAG
CCGGGCACATCAGCGCCTGGCAGCAGTGGCGTCTGGCGGAAAACCTCAGTGTGACGCTCCCCGCCGCGTC
CCACGCCATCCCGCATCTGACCACCAGCGAAATGGATTTTTGCATCGAGCTGGGTAATAAGCGTTGGCAA
TTTAACCGCCAGTCAGGCTTTCTTTCACAGATGTGGATTGGCGATAAAAAACAACTGCTGACGCCGCTGC
GCGATCAGTTCACCCGTGCACCGCTGGATAACGACATTGGCGTAAGTGAAGCGACCCGCATTGACCCTAA
CGCCTGGGTCGAACGCTGGAAGGCGGCGGGCCATTACCAGGCCGAAGCAGCGTTGTTGCAGTGCACGGCA
GATACACTTGCTGATGCGGTGCTGATTACGACCGCTCACGCGTGGCAGCATCAGGGGAAAACCTTATTTA
TCAGCCGGAAAACCTACCGGATTGATGGTAGTGGTCAAATGGCGATTACCGTTGATGTTGAAGTGGCGAG
CGATACACCGCATCCGGCGCGGATTGGCCTGAACTGCCAGCTGGCGCAGGTAGCAGAGCGGGTAAACTGG
CTCGGATTAGGGCCGCAAGAAAACTATCCCGACCGCCTTACTGCCGCCTGTTTTGACCGCTGGGATCTGC
CATTGTCAGACATGTATACCCCGTACGTCTTCCCGAGCGAAAACGGTCTGCGCTGCGGGACGCGCGAATT
GAATTATGGCCCACACCAGTGGCGCGGCGACTTCCAGTTCAACATCAGCCGCTACAGTCAACAGCAACTG
ATGGAAACCAGCCATCGCCATCTGCTGCACGCGGAAGAAGGCACATGGCTGAATATCGACGGTTTCCATA
TGGGGATTGGTGGCGACGACTCCTGGAGCCCGTCAGTATCGGCGGAGTTCCAGCTGAGCGCCGGTCGCTA
CCATTACCAGTTGGTCTGGTGTCAAAAAACTAAGGGTGGGCGCGCCGACCCAGCTTTCTTGTACAAAGTG
GTTGATCTAGAGGGCCCGCGGTTCGAAGGTAAGCCTATCCCTAACCCTCTCCTCGGTCTCGATTCTACGC
GTACCGGTTAGTAATGAGTTTAAACGGGGGAGGCTAACTGAAACACGGAAGGAGACAATACCGGAAGGAA
CCCGCGCTATGACGGCAATAAAAAGACAGAATAAAACGCACGGGTGTTGGGTCGTTTGTTCATAAACGCG
GGGTTCGGTCCCAGGGCTGGCACTCTGTCGATACCCCACCGAGACCCCATTGGGGCCAATACGCCCGCGT
TTCTTCCTTTTCCCCACCCCACCCCCCAAGTTCGGGTGAAGGCCCAGGGCTCGCAGCCAACGTCGGGGCG
GCAGGCCCTGCCATAGCAGATCCGATTCGACAGATCACTGAAATGTGTGGGCGTGGCTTAAGGGTGGGAA
AGAATATATAAGGTGGGGGTCTTATGTAGTTTTGTATCTGTTTTGCAGCAGCCGCCGCCGCCATGAGCAC
CAACTCGTTTGATGGAAGCATTGTGAGCTCATATTTGACAACGCGCATGCCCCCATGGGCCGGGGTGCGT
CAGAATGTGATGGGCTCCAGCATTGATGGTCGCCCCGTCCTGCCCGCAAACTCTACTACCTTGACCTACG
AGACCGTGTCTGGAACGCCGTTGGAGACTGCAGCCTCCGCCGCCGCTTCAGCCGCTGCAGCCACCGCCCG
CGGGATTGTGACTGACTTTGCTTTCCTGAGCCCGCTTGCAAGCAGTGCAGCTTCCCGTTCATCCGCCCGC
GATGACAAGTTGACGGCTCTTTTGGCACAATTGGATTCTTTGACCCGGGAACTTAATGTCGTTTCTCAGC
AGCTGTTGGATCTGCGCCAGCAGGTTTCTGCCCTGAAGGCTTCCTCCCCTCCCAATGCGGTTTAAAACAT
AAATAAAAAACCAGACTCTGTTTGGATTTGGATCAAGCAAGTGTCTTGCTGTCTTTATTTAGGGGTTTTG
CGCGCGCGGTAGGCCCGGGACCAGCGGTCTCGGTCGTTGAGGGTCCTGTGTATTTTTTCCAGGACGTGGT
AAAGGTGACTCTGGATGTTCAGATACATGGGCATAAGCCCGTCTCTGGGGTGGAGGTAGCACCACTGCAG
AGCTTCATGCTGCGGGGTGGTGTTGTAGATGATCCAGTCGTAGCAGGAGCGCTGGGCGTGGTGCCTAAAA
ATGTCTTTCAGTAGCAAGCTGATTGCCAGGGGCAGGCCCTTGGTGTAAGTGTTTACAAAGCGGTTAAGCT
GGGATGGGTGCATACGTGGGGATATGAGATGCATCTTGGACTGTATTTTTAGGTTGGCTATGTTCCCAGC
CATATCCCTCCGGGGATTCATGTTGTGCAGAACCACCAGCACAGTGTATCCGGTGCACTTGGGAAATTTG
TCATGTAGCTTAGAAGGAAATGCGTGGAAGAACTTGGAGACGCCCTTGTGACCTCCAAGATTTTCCATGC
ATTCGTCCATAATGATGGCAATGGGCCCACGGGCGGCGGCCTGGGCGAAGATATTTCTGGGATCACTAAC
GTCATAGTTGTGTTCCAGGATGAGATCGTCATAGGCCATTTTTACAAAGCGCGGGCGGAGGGTGCCAGAC
TGCGGTATAATGGTTCCATCCGGCCCAGGGGCGTAGTTACCCTCACAGATTTGCATTTCCCACGCTTTGA
GTTCAGATGGGGGGATCATGTCTACCTGCGGGGCGATGAAGAAAACGGTTTCCGGGGTAGGGGAGATCAG
CTGGGAAGAAAGCAGGTTCCTGAGCAGCTGCGACTTACCGCAGCCGGTGGGCCCGTAAATCACACCTATT
ACCGGGTGCAACTGGTAGTTAAGAGAGCTGCAGCTGCCGTCATCCCTGAGCAGGGGGGCCACTTCGTTAA
GCATGTCCCTGACTCGCATGTTTTCCCTGACCAAATCCGCCAGAAGGCGCTCGCCGCCCAGCGATAGCAG
TTCTTGCAAGGAAGCAAAGTTTTTCAACGGTTTGAGACCGTCCGCCGTAGGCATGCTTTTGAGCGTTTGA
CCAAGCAGTTCCAGGCGGTCCCACAGCTCGGTCACCTGCTCTACGGCATCTCGATCCAGCATATCTCCTC
GTTTCGCGGGTTGGGGCGGCTTTCGCTGTACGGCAGTAGTCGGTGCTCGTCCAGACGGGCCAGGGTCATG
TCTTTCCACGGGCGCAGGGTCCTCGTCAGCGTAGTCTGGGTCACGGTGAAGGGGTGCGCTCCGGGCTGCG
CGCTGGCCAGGGTGCGCTTGAGGCTGGTCCTGCTGGTGCTGAAGCGCTGCCGGTCTTCGCCCTGCGCGTC
GGCCAGGTAGCATTTGACCATGGTGTCATAGTCCAGCCCCTCCGCGGCGTGGCCCTTGGCGCGCAGCTTG
CCCTTGGAGGAGGCGCCGCACGAGGGGCAGTGCAGACTTTTGAGGGCGTAGAGCTTGGGCGCGAGAAATA
CCGATTCCGGGGAGTAGGCATCCGCGCCGCAGGCCCCGCAGACGGTCTCGCATTCCACGAGCCAGGTGAG
CTCTGGCCGTTCGGGGTCAAAAACCAGGTTTCCCCCATGCTTTTTGATGCGTTTCTTACCTCTGGTTTCC
ATGAGCCGGTGTCCACGCTCGGTGACGAAAAGGCTGTCCGTGTCCCCGTATACAGACTTGAGAGGCCTGT
CCTCGAGCGGTGTTCCGCGGTCCTCCTCGTATAGAAACTCGGACCACTCTGAGACAAAGGCTCGCGTCCA
GGCCAGCACGAAGGAGGCTAAGTGGGAGGGGTAGCGGTCGTTGTCCACTAGGGGGTCCACTCGCTCCAGG
GTGTGAAGACACATGTCGCCCTCTTCGGCATCAAGGAAGGTGATTGGTTTGTAGGTGTAGGCCACGTGAC
CGGGTGTTCCTGAAGGGGGGCTATAAAAGGGGGTGGGGGCGCGTTCGTCCTCACTCTCTTCCGCATCGCT
GTCTGCGAGGGCCAGCTGTTGGGGTGAGTACTCCCTCTGAAAAGCGGGCATGACTTCTGCGCTAAGATTG
TCAGTTTCCAAAAACGAGGAGGATTTGATATTCACCTGGCCCGCGGTGATGCCTTTGAGGGTGGCCGCAT
CCATCTGGTCAGAAAAGACAATCTTTTTGTTGTCAAGCTTGGTGGCAAACGACCCGTAGAGGGCGTTGGA
CAGCAACTTGGCGATGGAGCGCAGGGTTTGGTTTTTGTCGCGATCGGCGCGCTCCTTGGCCGCGATGTTT
AGCTGCACGTATTCGCGCGCAACGCACCGCCATTCGGGAAAGACGGTGGTGCGCTCGTCGGGCACCAGGT
GCACGCGCCAACCGCGGTTGTGCAGGGTGACAAGGTCAACGCTGGTGGCTACCTCTCCGCGTAGGCGCTC
GTTGGTCCAGCAGAGGCGGCCGCCCTTGCGCGAGCAGAATGGCGGTAGGGGGTCTAGCTGCGTCTCGTCC
GGGGGGTCTGCGTCCACGGTAAAGACCCCGGGCAGCAGGCGCGCGTCGAAGTAGTCTATCTTGCATCCTT
GCAAGTCTAGCGCCTGCTGCCATGCGCGGGCGGCAAGCGCGCGCTCGTATGGGTTGAGTGGGGGACCCCA
TGGCATGGGGTGGGTGAGCGCGGAGGCGTACATGCCGCAAATGTCGTAAACGTAGAGGGGCTCTCTGAGT
ATTCCAAGATATGTAGGGTAGCATCTTCCACCGCGGATGCTGGCGCGCACGTAATCGTATAGTTCGTGCG
AGGGAGCGAGGAGGTCGGGACCGAGGTTGCTACGGGCGGGCTGCTCTGCTCGGAAGACTATCTGCCTGAA
GATGGCATGTGAGTTGGATGATATGGTTGGACGCTGGAAGACGTTGAAGCTGGCGTCTGTGAGACCTACC
GCGTCACGCACGAAGGAGGCGTAGGAGTCGCGCAGCTTGTTGACCAGCTCGGCGGTGACCTGCACGTCTA
GGGCGCAGTAGTCCAGGGTTTCCTTGATGATGTCATACTTATCCTGTCCCTTTTTTTTCCACAGCTCGCG
GTTGAGGACAAACTCTTCGCGGTCTTTCCAGTACTCTTGGATCGGAAACCCGTCGGCCTCCGAACGGTAA
GAGCCTAGCATGTAGAACTGGTTGACGGCCTGGTAGGCGCAGCATCCCTTTTCTACGGGTAGCGCGTATG
CCTGCGCGGCCTTCCGGAGCGAGGTGTGGGTGAGCGCAAAGGTGTCCCTGACCATGACTTTGAGGTACTG
GTATTTGAAGTCAGTGTCGTCGCATCCGCCCTGCTCCCAGAGCAAAAAGTCCGTGCGCTTTTTGGAACGC
GGATTTGGCAGGGCGAAGGTGACATCGTTGAAGAGTATCTTTCCCGCGCGAGGCATAAAGTTGCGTGTGA
TGCGGAAGGGTCCCGGCACCTCGGAACGGTTGTTAATTACCTGGGCGGCGAGCACGATCTCGTCAAAGCC
GTTGATGTTGTGGCCCACAATGTAAAGTTCCAAGAAGCGCGGGATGCCCTTGATGGAAGGCAATTTTTTA
AGTTCCTCGTAGGTGAGCTCTTCAGGGGAGCTGAGCCCGTGCTCTGAAAGGGCCCAGTCTGCAAGATGAG
GGTTGGAAGCGACGAATGAGCTCCACAGGTCACGGGCCATTAGCATTTGCAGGTGGTCGCGAAAGGTCCT
AAACTGGCGACCTATGGCCATTTTTTCTGGGGTGATGCAGTAGAAGGTAAGCGGGTCTTGTTCCCAGCGG
TCCCATCCAAGGTTCGCGGCTAGGTCTCGCGCGGCAGTCACTAGAGGCTCATCTCCGCCGAACTTCATGA
CCAGCATGAAGGGCACGAGCTGCTTCCCAAAGGCCCCCATCCAAGTATAGGTCTCTACATCGTAGGTGAC
AAAGAGACGCTCGGTGCGAGGATGCGAGCCGATCGGGAAGAACTGGATCTCCCGCCACCAATTGGAGGAG
TGGCTATTGATGTGGTGAAAGTAGAAGTCCCTGCGACGGGCCGAACACTCGTGCTGGCTTTTGTAAAAAC
GTGCGCAGTACTGGCAGCGGTGCACGGGCTGTACATCCTGCACGAGGTTGACCTGACGACCGCGCACAAG
GAAGCAGAGTGGGAATTTGAGCCCCTCGCCTGGCGGGTTTGGCTGGTGGTCTTCTACTTCGGCTGCTTGT
CCTTGACCGTCTGGCTGCTCGAGGGGAGTTACGGTGGATCGGACCACCACGCCGCGCGAGCCCAAAGTCC
AGATGTCCGCGCGCGGCGGTCGGAGCTTGATGACAACATCGCGCAGATGGGAGCTGTCCATGGTCTGGAG
CTCCCGCGGCGTCAGGTCAGGCGGGAGCTCCTGCAGGTTTACCTCGCATAGACGGGTCAGGGCGCGGGCT
AGATCCAGGTGATACCTAATTTCCAGGGGCTGGTTGGTGGCGGCGTCGATGGCTTGCAAGAGGCCGCATC
CCCGCGGCGCGACTACGGTACCGCGCGGCGGGCGGTGGGCCGCGGGGGTGTCCTTGGATGATGCATCTAA
AAGCGGTGACGCGGGCGAGCCCCCGGAGGTAGGGGGGGCTCCGGACCCGCCGGGAGAGGGGGCAGGGGCA
CGTCGGCGCCGCGCGCGGGCAGGAGCTGGTGCTGCGCGCGTAGGTTGCTGGCGAACGCGACGACGCGGCG
GTTGATCTCCTGAATCTGGCGCCTCTGCGTGAAGACGACGGGCCCGGTGAGCTTGAGCCTGAAAGAGAGT
TCGACAGAATCAATTTCGGTGTCGTTGACGGCGGCCTGGCGCAAAATCTCCTGCACGTCTCCTGAGTTGT
CTTGATAGGCGATCTCGGCCATGAACTGCTCGATCTCTTCCTCCTGGAGATCTCCGCGTCCGGCTCGCTC
CACGGTGGCGGCGAGGTCGTTGGAAATGCGGGCCATGAGCTGCGAGAAGGCGTTGAGGCCTCCCTCGTTC
CAGACGCGGCTGTAGACCACGCCCCCTTCGGCATCGCGGGCGCGCATGACCACCTGCGCGAGATTGAGCT
CCACGTGCCGGGCGAAGACGGCGTAGTTTCGCAGGCGCTGAAAGAGGTAGTTGAGGGTGGTGGCGGTGTG
TTCTGCCACGAAGAAGTACATAACCCAGCGTCGCAACGTGGATTCGTTGATATCCCCCAAGGCCTCAAGG
CGCTCCATGGCCTCGTAGAAGTCCACGGCGAAGTTGAAAAACTGGGAGTTGCGCGCCGACACGGTTAACT
CCTCCTCCAGAAGACGGATGAGCTCGGCGACAGTGTCGCGCACCTCGCGCTCAAAGGCTACAGGGGCCTC
TTCTTCTTCTTCAATCTCCTCTTCCATAAGGGCCTCCCCTTCTTCTTCTTCTGGCGGCGGTGGGGGAGGG
GGGACACGGCGGCGACGACGGCGCACCGGGAGGCGGTCGACAAAGCGCTCGATCATCTCCCCGCGGCGAC
GGCGCATGGTCTCGGTGACGGCGCGGCCGTTCTCGCGGGGGCGCAGTTGGAAGACGCCGCCCGTCATGTC
CCGGTTATGGGTTGGCGGGGGGCTGCCATGCGGCAGGGATACGGCGCTAACGATGCATCTCAACAATTGT
TGTGTAGGTACTCCGCCGCCGAGGGACCTGAGCGAGTCCGCATCGACCGGATCGGAAAACCTCTCGAGAA
AGGCGTCTAACCAGTCACAGTCGCAAGGTAGGCTGAGCACCGTGGCGGGCGGCAGCGGGCGGCGGTCGGG
GTTGTTTCTGGCGGAGGTGCTGCTGATGATGTAATTAAAGTAGGCGGTCTTGAGACGGCGGATGGTCGAC
AGAAGCACCATGTCCTTGGGTCCGGCCTGCTGAATGCGCAGGCGGTCGGCCATGCCCCAGGCTTCGTTTT
GACATCGGCGCAGGTCTTTGTAGTAGTCTTGCATGAGCCTTTCTACCGGCACTTCTTCTTCTCCTTCCTC
TTGTCCTGCATCTCTTGCATCTATCGCTGCGGCGGCGGCGGAGTTTGGCCGTAGGTGGCGCCCTCTTCCT
CCCATGCGTGTGACCCCGAAGCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCACAAAGCGGTGGTATGCGCC
CGTGTTGATGGTGTAAGTGCAGTTGGCCATAACGGACCAGTTAACGGTCTGGTGACCCGGCTGCGAGAGC
TCGGTGTACCTGAGACGCGAGTAAGCCCTCGAGTCAAATACGTAGTCGTTGCAAGTCCGCACCAGGTACT
GGTATCCCACCAAAAAGTGCGGCGGCGGCTGGCGGTAGAGGGGCCAGCGTAGGGTGGCCGGGGCTCCGGG
GGCGAGATCTTCCAACATAAGGCGATGATATCCGTAGATGTACCTGGACATCCAGGTGATGCCGGCGGCG
GTGGTGGAGGCGCGCGGAAAGTCGCGGACGCGGTTCCAGATGTTGCGCAGCGGCAAAAAGTGCTCCATGG
TCGGGACGCTCTGGCCGGTCAGGCGCGCGCAATCGTTGACGCTCTAGACCGTGCAAAAGGAGAGCCTGTA
AGCGGGCACTCTTCCGTGGTCTGGTGGATAAATTCGCAAGGGTATCATGGCGGACGACCGGGGTTCGAGC
CCCGTATCCGGCCGTCCGCCGTGATCCATGCGGTTACCGCCCGCGTGTCGAACCCAGGTGTGCGACGTCA
GACAACGGGGGAGTGCTCCTTTTGGCTTCCTTCCAGGCGCGGCGGCTGCTGCGCTAGCTTTTTTGGCCAC
TGGCCGCGCGCAGCGTAAGCGGTTAGGCTGGAAAGCGAAAGCATTAAGTGGCTCGCTCCCTGTAGCCGGA
GGGTTATTTTCCAAGGGTTGAGTCGCGGGACCCCCGGTTCGAGTCTCGGACCGGCCGGACTGCGGCGAAC
GGGGGTTTGCCTCCCCGTCATGCAAGACCCCGCTTGCAAATTCCTCCGGAAACAGGGACGAGCCCCTTTT
TTGCTTTTCCCAGATGCATCCGGTGCTGCGGCAGATGCGCCCCCCTCCTCAGCAGCGGCAAGACCAAGAG
CAGCGGCAGACATGCAGGGCACCCTCCCCTCCTCCTACCGCGTCAGGAGGGGCGACATCCGCGGTTGACG
CGGCAGCAGATGGTGATTACGAACCCCCGCGGCGCCGGGCCCGGCACTACCTGGACTTGGAGGAGGGCGA
GGGCCTGGCGCGGCTAGGAGCGCCCTCTCCTGAGCGGTACCCAAGGGTGCAGCTGAAGCGTGATACGCGT
GAGGCGTACGTGCCGCGGCAGAACCTGTTTCGCGACCGCGAGGGAGAGGAGCCCGAGGAGATGCGGGATC
GAAAGTTCCACGCAGGGCGCGAGCTGCGGCATGGCCTGAATCGCGAGCGGTTGCTGCGCGAGGAGGACTT
TGAGCCCGACGCGCGAACCGGGATTAGTCCCGCGCGCGCACACGTGGCGGCCGCCGACCTGGTAACCGCA
TACGAGCAGACGGTGAACCAGGAGATTAACTTTCAAAAAAGCTTTAACAACCACGTGCGTACGCTTGTGG
CGCGCGAGGAGGTGGCTATAGGACTGATGCATCTGTGGGACTTTGTAAGCGCGCTGGAGCAAAACCCAAA
TAGCAAGCCGCTCATGGCGCAGCTGTTCCTTATAGTGCAGCACAGCAGGGACAACGAGGCATTCAGGGAT
GCGCTGCTAAACATAGTAGAGCCCGAGGGCCGCTGGCTGCTCGATTTGATAAACATCCTGCAGAGCATAG
TGGTGCAGGAGCGCAGCTTGAGCCTGGCTGACAAGGTGGCCGCCATCAACTATTCCATGCTTAGCCTGGG
CAAGTTTTACGCCCGCAAGATATACCATACCCCTTACGTTCCCATAGACAAGGAGGTAAAGATCGAGGGG
TTCTACATGCGCATGGCGCTGAAGGTGCTTACCTTGAGCGACGACCTGGGCGTTTATCGCAACGAGCGCA
TCCACAAGGCCGTGAGCGTGAGCCGGCGGCGCGAGCTCAGCGACCGCGAGCTGATGCACAGCCTGCAAAG
GGCCCTGGCTGGCACGGGCAGCGGCGATAGAGAGGCCGAGTCCTACTTTGACGCGGGCGCTGACCTGCGC
TGGGCCCCAAGCCGACGCGCCCTGGAGGCAGCTGGGGCCGGACCTGGGCTGGCGGTGGCACCCGCGCGCG
CTGGCAACGTCGGCGGCGTGGAGGAATATGACGAGGACGATGAGTACGAGCCAGAGGACGGCGAGTACTA
AGCGGTGATGTTTCTGATCAGATGATGCAAGACGCAACGGACCCGGCGGTGCGGGCGGCGCTGCAGAGCC
AGCCGTCCGGCCTTAACTCCACGGACGACTGGCGCCAGGTCATGGACCGCATCATGTCGCTGACTGCGCG
CAATCCTGACGCGTTCCGGCAGCAGCCGCAGGCCAACCGGCTCTCCGCAATTCTGGAAGCGGTGGTCCCG
GCGCGCGCAAACCCCACGCACGAGAAGGTGCTGGCGATCGTAAACGCGCTGGCCGAAPACAGGGCCATCC
GGCCCGACGAGGCCGGCCTGGTCTACGACGCGCTGCTTCAGCGCGTGGCTCGTTACAACAGCGGCAACGT
GCAGACCAACCTGGACCGGCTGGTGGGGGATGTGCGCGAGGCCGTGGCGCAGCGTGAGCGCGCGCAGCAG
CAGGGCAACCTGGGCTCCATGGTTGCACTAAACGCCTTCCTGAGTACACAGCCCGCCAACGTGCCGCGGG
GACAGGAGGACTACACCAACTTTGTGAGCGCACTGCGGCTAATGGTGACTGAGACACCGCAAAGTGAGGT
GTACCAGTCTGGGCCAGACTATTTTTTCCAGACCAGTAGACAAGGCCTGCAGACCGTAAACCTGAGCCAG
GCTTTCAAAAACTTGCAGGGGCTGTGGGGGGTGCGGGCTCCCACAGGCGACCGCGCGACCGTGTCTAGCT
TGCTGACGCCCAACTCGCGCCTGTTGCTGCTGCTAATAGCGCCCTTCACGGACAGTGGCAGCGTGTCCCG
GGACACATACCTAGGTCACTTGCTGACACTGTACCGCGAGGCCATAGGTCAGGCGCATGTGGACGAGCAT
ACTTTCCAGGAGATTACAAGTGTCAGCCGCGCGCTGGGGCAGGAGGACACGGGCAGCCTGGAGGCAACCC
TAAACTACCTGCTGACCAACCGGCGGCAGAAGATCCCCTCGTTGCACAGTTTAAACAGCGAGGAGGAGCG
CATTTTGCGCTACGTGCAGCAGAGCGTGAGCCTTAACCTGATGCGCGACGGGGTAACGCCCAGCGTGGCG
CTGGACATGACCGCGCGCAACATGGAACCGGGCATGTATGCCTCAAACCGGCCGTTTATCAACCGCCTAA
TGGACTACTTGCATCGCGCGGCCGCCGTGAACCCCGAGTATTTCACCAATGCCATCTTGAACCCGCACTG
GCTACCGCCCCCTGGTTTCTACACCGGGGGATTCGAGGTGCCCGAGGGTAACGATGGATTCCTCTGGGAC
GACATAGACGACAGCGTGTTTTCCCCGCAACCGCAGACCCTGCTAGAGTTGCAACAGCGCGAGCAGGCAG
AGGCGGCGCTGCGAAAGGAAAGCTTCCGCAGGCCAAGCAGCTTGTCCGATCTAGGCGCTGCGGCCCCGCG
GTCAGATGCTAGTAGCCCATTTCCAAGCTTGATAGGGTCTCTTACCAGCACTCGCACCACCCGCCCGCGC
CTGCTGGGCGAGGAGGAGTACCTAAACAACTCGCTGCTGCAGCCGCAGCGCGAAAAAAACCTGCCTCCGG
CATTTCCCAACAACGGGATAGAGAGCCTAGTGGACAAGATGAGTAGATGGAAGACGTACGCGCAGGAGCA
CAGGGACGTGCCAGGCCCGCGCCCGCCCACCCGTCGTCAAAGGCACGACCGTCAGCGGGGTCTGGTGTGG
GAGGACGATGACTCGGCAGACGACAGCAGCGTCCTGGATTTGGGAGGGAGTGGCAACCCGTTTGCGCACC
TTCGCCCCAGGCTGGGGAGAATGTTTTAAAAAAAAAAAAGCATGATGCAAAATAAAAAACTCACCAAGGC
CATGGCACCGAGCGTTGGTTTTCTTGTATTCCCCTTAGTATGCGGCGCGCGGCGATGTATGAGGAAGGTC
CTCCTCCCTCCTACGAGAGTGTGGTGAGCGCGGCGCCAGTGGCGGCGGCGCTGGGTTCTCCCTTCGATGC
TCCCCTGGACCCGCCGTTTGTGCCTCCGCGGTACCTGCGGCCTACCGGGGGGAGAAACAGCATCCGTTAC
TCTGAGTTGGCACCCCTATTCGACACCACCCGTGTGTACCTGGTGGACAACAAGTCAACGGATGTGGCAT
CCCTGAACTACCAGAACGACCACAGCAACTTTCTGACCACGGTCATTCAAAACAATGACTACAGCCCGGG
GGAGGCAAGCACACAGACCATCAATCTTGACGACCGGTCGCACTGGGGCGGCGACCTGAAAACCATCCTG
CATACCAACATGCCAAATGTGAACGAGTTCATGTTTACCAATAAGTTTAAGGCGCGGGTGATGGTGTCGC
GCTTGCCTACTAAGGACAATCAGGTGGAGCTGAAATACGAGTGGGTGGAGTTCACGCTGCCCGAGGGCAA
CTACTCCGAGACCATGACCATAGACCTTATGAACAACGCGATCGTGGAGCACTACTTGAAAGTGGGCAGA
CAGAACGGGGTTCTGGAAAGCGACATCGGGGTAAAGTTTGACACCCGCAACTTCAGACTGGGGTTTGACC
CCGTCACTGGTCTTGTCATGCCTGGGGTATATACAAACGAAGCCTTCCATCCAGACATCATTTTGCTGCC
AGGATGCGGGGTGGACTTCACCCACAGCCGCCTGAGCAACTTGTTGGGCATCCGCAAGCGGCAACCCTTC
CAGGAGGGCTTTAGGATCACCTACGATGATCTGGAGGGTGGTAACATTCCCGCACTGTTGGATGTGGACG
CCTACCAGGCGAGCTTGAAAGATGACACCGAACAGGGCGGGGGTGGCGCAGGCGGCAGCAACAGCAGTGG
CAGCGGCGCGGAAGAGAACTCCAACGCGGCAGCCGCGGCAATGCAGCCGGTGGAGGACATGAACGATCAT
GCCATTCGCGGCGACACCTTTGCCACACGGGCTGAGGAGAAGCGCGCTGAGGCCGAAGCAGCGGCCGAAG
CTGCCGCCCCCGCTGCGCAACCCGAGGTCGAGAAGCCTCAGAAGAAACCGGTGATCAAACCCCTGACAGA
GGACAGCAAGAAACGCAGTTACAACCTAATAAGCAATGACAGCACCTTCACCCAGTACCGCAGCTGGTAC
CTTGCATACAACTACGGCGACCCTCAGACCGGAATCCGCTCATGGACCCTGCTTTGCACTCCTGACGTAA
CCTGCGGCTCGGAGCAGGTCTACTGGTCGTTGCCAGACATGATGCAAGACCCCGTGACCTTCCGCTCCAC
GCGCCAGATCAGCAACTTTCCGGTGGTGGGCGCCGAGCTGTTGCCCGTGCACTCCAAGAGCTTCTACAAC
GACCAGGCCGTCTACTCCCAACTCATCCGCCAGTTTACCTCTCTGACCCACGTGTTCAATCGCTTTCCCG
AGAACCAGATTTTGGCGCGCCCGCCAGCCCCCACCATCACCACCGTCAGTGAAAACGTTCCTGCTCTCAC
AGATCACGGGACGCTACCGCTGCGCAACAGCATCGGAGGAGTCCAGCGAGTGACCATTACTGACGCCAGA
CGCCGCACCTGCCCCTACGTTTACAAGGCCCTGGGCATAGTCTCGCCGCGCGTCCTATCGAGCCGCACTT
TTTGAGCAAGCATGTCCATCCTTATATCGCCCAGCAATAACACAGGCTGGGGCCTGCGCTTCCCAAGCAA
GATGTTTGGCGGGGCCAAGAAGCGCTCCGACCAACACCCAGTGCGCGTGCGCGGGCACTACCGCGCGCCC
TGGGGCGCGCACAAACGCGGCCGCACTGGGCGCACCACCGTCGATGACGCCATCGACGCGGTGGTGGAGG
AGGCGCGCAACTACACGCCCACGCCGCCACCAGTGTCCACAGTGGACGCGGCCATTCAGACCGTGGTGCG
CGGAGCCCGGCGCTATGCTAAAATGAAGAGACGGCGGAGGCGCGTAGCACGTCGCCACCGCCGCCGACCC
GGCACTGCCGCCCAACGCGCGGCGGCGGCCCTGCTTAACCGCGCACGTCGCACCGGCCGACGGGCGGCCA
TGCGGGCCGCTCGAAGGCTGGCCGCGGGTATTGTCACTGTGCCCCCCAGGTCCAGGCGACGAGCGGCCGC
CGCAGCAGCCGCGGCCATTAGTGCTATGACTCAGGGTCGCAGGGGCAACGTGTATTGGGTGCGCGACTCG
GTTAGCGGCCTGCGCGTGCCCGTGCGCACCCGCCCCCCGCGCAACTAGATTGCAAGAAAAAACTACTTAG
ACTCGTACTGTTGTATGTATCCAGCGGCGGCGGCGCGCAACGAAGCTATGTCCAAGCGCAAAATCAAAGA
AGAGATGCTCCAGGTCATCGCGCCGGAGATCTATGGCCCCCCGAAGAAGGAAGAGCAGGATTACAAGCCC
CGAAAGCTAAAGCGGGTCAAAAAGAAAAAGAAAGATGATGATGATGAACTTGACGACGAGGTGGAACTGC
TGCACGCTACCGCGCCCAGGCGACGGGTACAGTGGAAAGGTCGACGCGTAAAACGTGTTTTGCGACCCGG
CACCACCGTAGTCTTTACGCCCGGTGAGCGCTCCACCCGCACCTACAAGCGCGTGTATGATGAGGTGTAC
GGCGACGAGGACCTGCTTGAGCAGGCCAACGAGCGCCTCGGGGAGTTTGCCTACGGAAAGCGGCATAAGG
ACATGCTGGCGTTGCCGCTGGACGAGGGCAACCCAACACCTAGCCTAAAGCCCGTAACACTGCAGCAGGT
GCTGCCCGCGCTTGCACCGTCCGAAGAAAAGCGCGGCCTAAAGCGCGAGTCTGGTGACTTGGCACCCACC
GTGCAGCTGATGGTACCCAAGCGCCAGCGACTGGAAGATGTCTTGGAAAAAATGACCGTGGAACCTGGGC
TGGAGCCCGAGGTCCGCGTGCGGCCAATCAAGCAGGTGGCGCCGGGACTGGGCGTGCAGACCGTGGACGT
TCAGATACCCACTACCAGTAGCACCAGTATTGCCACCGCCACAGAGGGCATGGAGACACAAACGTCCCCG
GTTGCCTCAGCGGTGGCGGATGCCGCGGTGCAGGCGGTCGCTGCGGCCGCGTCCAAGACCTCTACGGAGG
TGCAAACGGACCCGTGGATGTTTCGCGTTTCAGCCCCCCGGCGCCCGCGCGGTTCGAGGAAGTACGGCGC
CGCCAGCGCGCTACTGCCCGAATATGCCCTACATCCTTCCATTGCGCCTACCCCCGGCTATCGTGGCTAC
ACCTACCGCCCCAGAAGACGAGCAACTACCCGACGCCGAACCACCACTGGAACCCGCCGCCGCCGTCGCC
GTCGCCAGCCCGTGCTGGCCCCGATTTCCGTGCGCAGGGTGGCTCGCGAAGGAGGCAGGACCCTGGTGCT
GCCAACAGCGCGCTACCACCCCAGCATCGTTTAAAAGCCGGTCTTTGTGGTTCTTGCAGATATGGCCCTC
ACCTGCCGCCTCCGTTTCCCGGTGCCGGGATTCCGAGGAAGAATGCACCGTAGGAGGGGCATGGCCGGCC
ACGGCCTGACGGGCGGCATGCGTCGTGCGCACCACCGGCGGCGGCGCGCGTCGCACCGTCGCATGCGCGG
CGGTATCCTGCCCCTCCTTATTCCACTGATCGCCGCGGCGATTGGCGCCGTGCCCGGAATTGCATCCGTG
GCCTTGCAGGCGCAGAGACACTGATTAAAAACAAGTTGCATGTGGAAAAATCAAAATAAAAAGTCTGGAC
TCTCACGCTCGCTTGGTCCTGTAACTATTTTGTAGAATGGAAGACATCAACTTTGCGTCTCTGGCCCCGC
GACACGGCTCGCGCCCGTTCATGGGAAACTGGCAAGATATCGGCACCAGCAATATGAGCGGTGGCGCCTT
CAGCTGGGGCTCGCTGTGGAGCGGCATTAAAAATTTCGGTTCCACCGTTAAGAACTATGGCAGCAAGGCC
TGGAACAGCAGCACAGGCCAGATGCTGAGGGATAAGTTGAAAGAGCAAAATTTCCAACAAAAGGTGGTAG
ATGGCCTGGCCTCTGGCATTAGCGGGGTGGTGGACCTGGCCAACCAGGCAGTGCAAAATAAGATTAACAG
TAAGCTTGATCCCCGCCCTCCCGTAGAGGAGCCTCCACCGGCCGTGGAGACAGTGTCTCCAGAGGGGCGT
GGCGAAAAGCGTCCGCGCCCCGACAGGGAAGAAACTCTGGTGACGCAAATAGACGAGCCTCCCTCGTACG
AGGAGGCACTAAAGCAAGGCCTGCCCACCACCCGTCCCATCGCGCCCATGGCTACCGGAGTGCTGGGCCA
GCACACACCCGTAACGCTGGACCTGCCTCCCCCCGCCGACACCCAGCAGAAACCTGTGCTGCCAGGCCCG
ACCGCCGTTGTTGTAACCCGTCCTAGCCGCGCGTCCCTGCGCCGCGCCGCCAGCGGTCCGCGATCGTTGC
GGCCCGTAGCCAGTGGCAACTGGCAAAGCACACTGAACAGCATCGTGGGTCTGGGGGTGCAATCCCTGAA
GCGCCGACGATGCTTCTGAATAGCTAACGTGTCGTATGTGTGTCATGTATGCGTCCATGTCGCCGCCAGA
GGAGCTGCTGAGCCGCCGCGCGCCCGCTTTCCAAGATGGCTACCCCTTCGATGATGCCGCAGTGGTCTTA
CATGCACATCTCGGGCCAGGACGCCTCGGAGTACCTGAGCCCCGGGCTGGTGCAGTTTGCCCGCGCCACC
GAGACGTACTTCAGCCTGAATAACAAGTTTAGAAACCCCACGGTGGCGCCTACGCACGACGTGACCACAG
ACCGGTCCCAGCGTTTGACGCTGCGGTTCATCCCTGTGGACCGTGAGGATACTGCGTACTCGTACAAGGC
GCGGTTCACCCTAGCTGTGGGTGATAACCGTGTGCTGGACATGGCTTCCACGTACTTTGACATCCGCGGC
GTGCTGGACAGGGGCCCTACTTTTAAGCCCTACTCTGGCACTGCCTACAACGCCCTGGCTCCCAAGGGTG
CCCCAAATCCTTGCGAATGGGATGAAGCTGCTACTGCTCTTGAAATAAACCTAGAAGAAGAGGACGATGA
CAACGAAGACGAAGTAGACGAGCAAGCTGAGCAGCAAAAAACTCACGTATTTGGGCAGGCGCCTTATTCT
GGTATAAATATTACAAAGGAGGGTATTCAAATAGGTGTCGAAGGTCAAACACCTAAATATGCCGATAAAA
CATTTCAACCTGAACCTCAAATAGGAGAATCTCAGTGGTACGAAACTGAAATTAATCATGCAGCTGGGAG
AGTCCTTAAAAAGACTACCCCAATGAAACCATGTTACGGTTCATATGCAAAACCCACAAATGAAAATGGA
GGGCAAGGCATTCTTGTAAAGCAACAAAATGGAAAGCTAGAAAGTCAAGTGGAAATGCAATTTTTCTCAA
CTACTGAGGCGACCGCAGGCAATGGTGATAACTTGACTCCTAAAGTGGTATTGTACAGTGAAGATGTAGA
TATAGAAACCCCAGACACTCATATTTCTTACATGCCCACTATTAAGGAAGGTAACTCACGAGAACTAATG
GGCCAACAATCTATGCCCAACAGGCCTAATTACATTGCTTTTAGGGACAATTTTATTGGTCTAATGTATT
ACAACAGCACGGGTAATATGGGTGTTCTGGCGGGCCAAGCATCGCAGTTGAATGCTGTTGTAGATTTGCA
AGACAGAAACACAGAGCTTTCATACCAGCTTTTGCTTGATTCCATTGGTGATAGAACCAGGTACTTTTCT
ATGTGGAATCAGGCTGTTGACAGCTATGATCCAGATGTTAGAATTATTGAAAATCATGGAACTGAAGATG
AACTTCCAAATTACTGCTTTCCACTGGGAGGTGTGATTAATACAGAGACTCTTACCAAGGTAAAACCTAA
AACAGGTCAGGAAAATGGATGGGAAAAAGATGCTACAGAATTTTCAGATAAAAATGAAATAAGAGTTGGA
AATAATTTTGCCATGGAAATCAATCTAAATGCCAACCTGTGGAGAAATTTCCTGTACTCCAACATAGCGC
TGTATTTGCCCGACAAGCTAAAGTACAGTCCTTCCAACGTAAAAATTTCTGATAACCCAAACACCTACGA
CTACATGAACAAGCGAGTGGTGGCTCCCGGGTTAGTGGACTGCTACATTAACCTTGGAGCACGCTGGTCC
CTTGACTATATGGACAACGTCAACCCATTTAACCACCACCGCAATGCTGGCCTGCGCTACCGCTCAATGT
TGCTGGGCAATGGTCGCTATGTGCCCTTCCACATCCAGGTGCCTCAGAAGTTCTTTGCCATTAAAAACCT
CCTTCTCCTGCCGGGCTCATACACCTACGAGTGGAACTTCAGGAAGGATGTTAACATGGTTCTGCAGAGC
TCCCTAGGAAATGACCTAAGGGTTGACGGAGCCAGCATTAAGTTTGATAGCATTTGCCTTTACGCCACCT
TCTTCCCCATGGCCCACAACACCGCCTCCACGCTTGAGGCCATGCTTAGAAACGACACCAACGACCAGTC
CTTTAACGACTATCTCTCCGCCGCCAACATGCTCTACCCTATACCCGCCAACGCTACCAACGTGCCCATA
TCCATCCCCTCCCGCAACTGGGCGGCTTTCCGCGGCTGGGCCTTCACGCGCCTTAAGACTAAGGAAACCC
CATCACTGGGCTCGGGCTACGACCCTTATTACACCTACTCTGGCTCTATACCCTACCTAGATGGAACCTT
TTACCTCAACCACACCTTTAAGAAGGTGGCCATTACCTTTGACTCTTCTGTCAGCTGGCCTGGCAATGAC
CGCCTGCTTACCCCCAACGAGTTTGAAATTAAGCGCTCAGTTGACGGGGAGGGTTACAACGTTGCCCAGT
GTAACATGACCAAAGACTGGTTCCTGGTACAAATGCTAGCTAACTACAACATTGGCTACCAGGGCTTCTA
TATCCCAGAGAGCTACAAGGACCGCATGTACTCCTTCTTTAGAAACTTCCAGCCCATGAGCCGTCAGGTG
GTGGATGATACTAAATACAAGGACTACCAACAGGTGGGCATCCTACACCAACACAACAACTCTGGATTTG
TTGGCTACCTTGCCCCCACCATGCGCGAAGGACAGGCCTACCCTGCTAACTTCCCCTATCCGCTTATAGG
CAAGACCGCAGTTGACAGCATTACCCAGAAAAAGTTTCTTTGCGATCGCACCCTTTGGCGCATCCCATTC
TCCAGTAACTTTATGTCCATGGGCGCACTCACAGACCTGGGCCAAAACCTTCTCTACGCCAACTCCGCCC
ACGCGCTAGACATGACTTTTGAGGTGGATCCCATGGACGAGCCCACCCTTCTTTATGTTTTGTTTGAAGT
CTTTGACGTGGTCCGTGTGCACCGGCCGCACCGCGGCGTCATCGAAACCGTGTACCTGCGCACGCCCTTC
TCGGCCGGCAACGCCACAACATAAAGAAGCAAGCAACATCAACAACAGCTGCCGCCATGGGCTCCAGTGA
GCAGGAACTGAAAGCCATTGTCAAAGATCTTGGTTCTGGGCCATATTTTTTGGGCACCTATGACAAGCGC
TTTCCAGGCTTTGTTTCTCCACACAAGCTCGCCTGCGCCATAGTCAATACGGCCGGTCGCGAGACTGGGG
GCGTACACTGGATGGCCTTTGCCTGGAACCCGCACTCAAAAACATGCTACCTCTTTGAGCCCTTTGGCTT
TTCTGACCAGCGACTCAAGCAGGTTTACCAGTTTGAGTACGAGTCACTCCTGCGCCGTAGCGCCATTGCT
TCTTCCCCCGACCGCTGTATAACGCTGGAAAAGTCCACCCAAAGCGTACAGGGGCCCAACTCGGCCGCCT
GTGGACTATTCTGCTGCATGTTTCTCCACGCCTTTGCCAACTGGCCCCAAACTCCCATGGATCACAACCC
CACCATGAACCTTATTACCGGGGTACCCAACTCCATGCTCAACAGTCCCCAGGTACAGCCCACCCTGCGT
CGCAACCAGGAACAGCTCTACAGCTTCCTGGAGCGCCACTCGCCCTACTTCCGCAGCCACAGTGCGCAGA
TTAGGAGCGCCACTTCTTTTTGTCACTTGAAAAACATGTAAAAATAATGTACTAGAGACACTTTCAATAA
AGGCAAATGCTTTTATTTGTACACTCTCGGGTGATTATTTACCCCCACCCTTGCCGTCTGCGCCGTTTAA
AAATCAAAGGGGTTCTGCCGCGCATCGCTATGCGCCACTGGCAGGGACACGTTGCGATACTGGTGTTTAG
TGCTCCACTTAAACTCAGGGACPACCATCCGCGGCAGCTCGGTGAAGTTTTCACTCCACAGGCTGCGCAC
CATCACCAACGCGTTTAGCAGGTCGGGCGCCGATATCTTGAAGTCGCAGTTGGGGCCTCCGCCCTGCGCG
CGCGAGTTGCGATACACAGGGTTGCAGCACTGGAACACTATCAGCGCCGGGTGGTGCACGCTGGCCAGCA
CGCTCTTGTCGGAGATCAGATCCGCGTCCAGGTCCTCCGCGTTGCTCAGGGCGAACGGAGTCAACTTTGG
TAGCTGCCTTCCCAAAAAGGGCGCGTGCCCAGGCTTTGAGTTGCACTCGCACCGTAGTGGCATCAAAAGG
TGACCGTGCCCGGTCTGGGCGTTAGGATACAGCGCCTGCATAAAAGCCTTGATCTGCTTAAAAGCCACCT
GAGCCTTTGCGCCTTCAGAGAAGAACATGCCGCAAGACTTGCCGGAAAACTGATTGGCCGGACAGGCCGC
GTCGTGCACGCAGCACCTTGCGTCGGTGTTGGAGATCTGCACCACATTTCGGCCCCACCGGTTCTTCACG
ATCTTGGCCTTGCTAGACTGCTCCTTCAGCGCGCGCTGCCCGTTTTCGCTCGTCACATCCATTTCAATCA
CGTGCTCCTTATTTATCATAATGCTTCCGTGTAGACACTTAAGCTCGCCTTCGATCTCAGCGCAGCGGTG
CAGCCACAACGCGCAGCCCGTGGGCTCGTGATGCTTGTAGGTCACCTCTGCAAACGACTGCAGGTACGCC
TGCAGGAATCGCCCCATCATCGTCACAAAGGTCTTGTTGCTGGTGAAGGTCAGCTGCAACCCGCGGTGCT
CCTCGTTCAGCCAGGTCTTGCATACGGCCGCCAGAGCTTCCACTTGGTCAGGCAGTAGTTTGAAGTTCGC
CTTTAGATCGTTATCCACGTGGTACTTGTCCATCAGCGCGCGCGCAGCCTCCATGCCCTTCTCCCACGCA
GACACGATCGGCACACTCAGCGGGTTCATCACCGTAATTTCACTTTCCGCTTCGCTGGGCTCTTCCTCTT
CCTCTTGCGTCCGCATACCACGCGCCACTGGGTCGTCTTCATTCAGCCGCCGCACTGTGCGCTTACCTCC
TTTGCCATGCTTGATTAGCACCGGTGGGTTGCTGAAACCCACCATTTGTAGCGCCACATCTTCTCTTTCT
TCCTCGCTGTCCACGATTACCTCTGGTGATGGCGGGCGCTCGGGCTTGGGAGAAGGGCGCTTCTTTTTCT
TCTTGGGCGCAATGGCCAAATCCGCCGCCGAGGTCGATGGCCGCGGGCTGGGTGTGCGCGGCACCAGCGC
GTCTTGTGATGAGTCTTCCTCGTCCTCGGACTCGATACGCCGCCTCATCCGCTTTTTTGGGGGCGCCCGG
GGAGGCGGCGGCGACGGGGACGGGGACGACACGTCCTCCATGGTTGGGGGACGTCGCGCCGCACCGCGTC
CGCGCTCGGGGGTGGTTTCGCGCTGCTCCTCTTCCCGACTGGCCATTTCCTTCTCCTATAGGCAGAAAAA
GATCATGGAGTCAGTCGAGAAGAAGGACAGCCTAACCGCCCCCTCTGAGTTCGCCACCACCGCCTCCACC
GATGCCGCCAACGCGCCTACCACCTTCCCCGTCGAGGCACCCCCGCTTGAGGAGGAGGAAGTGATTATCG
AGCAGGACCCAGGTTTTGTAAGCGAAGACGACGAGGACCGCTCAGTACCAACAGAGGATAAAAAGCAAGA
CCAGGACAACGCAGAGGCAAACGAGGAACAAGTCGGGCGGGGGGACGAAAGGCATGGCGACTACCTAGAT
GTGGGAGACGACGTGCTGTTGAAGCATCTGCAGCGCCAGTGCGCCATTATCTGCGACGCGTTGCAAGAGC
GCAGCGATGTGCCCCTCGCCATAGCGGATGTCAGCCTTGCCTACGAACGCCACCTATTCTCACCGCGCGT
ACCCCCCAAACGCCAAGAkAACGGCACATGCGAGCCCAACCCGCGCCTCAACTTCTACCCCGTATTTGCC
GTGCCAGAGGTGCTTGCCACCTATCACATCTTTTTCCAAAACTGCAAGATACCCCTATCCTGCCGTGCCA
ACCGCAGCCGAGCGGACAAGCAGCTGGCCTTGCGGCAGGGCGCTGTCATACCTGATATCGCCTCGCTCAA
CGAAGTGCCAAAAATCTTTGAGGGTCTTGGACGCGACGAGAAGCGCGCGGCAAACGCTCTGCAACAGGAA
AACAGCGAAAATGAAAGTCACTCTGGAGTGTTGGTGGAACTCGAGGGTGACAACGCGCGCCTAGCCGTAC
TAAAACGCAGCATCGAGGTCACCCACTTTGCCTACCCGGCACTTAACCTACCCCCCAAGGTCATGAGCAC
AGTCATGAGTGAGCTGATCGTGCGCCGTGCGCAGCCCCTGGAGAGGGATGCAAATTTGCAAGAACAAACA
GAGGAGGGCCTACCCGCAGTTGGCGACGAGCAGCTAGCGCGCTGGCTTCAAACGCGCGAGCCTGCCGACT
TGGAGGAGCGACGCAAACTAATGATGGCCGCAGTGCTCGTTACCGTGGAGCTTGAGTGCATGCAGCGGTT
CTTTGCTGACCCGGAGATGCAGCGCAAGCTAGAGGAAACATTGCACTACACCTTTCGACAGGGCTACGTA
CGCCAGGCCTGCAAGATCTCCAACGTGGAGCTCTGCAACCTGGTCTCCTACCTTGGAATTTTGCACGAAA
ACCGCCTTGGGCAAAACGTGCTTCATTCCACGCTCAAGGGCGAGGCGCGCCGCGACTACGTCCGCGACTG
CGTTTACTTATTTCTATGCTACACCTGGCAGACGGCCATGGGCGTTTGGCAGCAGTGCTTGGAGGAGTGC
AACCTCAAGGAGCTGCAGAAACTGCTAAAGCAAAACTTGAAGGACCTATGGACGGCCTTCAACGAGCGCT
CCGTGGCCGCGCACCTGGCGGACATCATTTTCCCCGAACGCCTGCTTAAAACCCTGCAACAGGGTCTGCC
AGACTTCACCAGTCAAAGCATGTTGCAGAACTTTAGGAACTTTATCCTAGAGCGCTCAGGAATCTTGCCC
GCCACCTGCTGTGCACTTCCTAGCGACTTTGTGCCCATTAAGTACCGCGAATGCCCTCCGCCGCTTTGGG
GCCACTGCTACCTTCTGCAGCTAGCCAACTACCTTGCCTACCACTCTGACATAATGGAAGACGTGAGCGG
TGACGGTCTACTGGAGTGTCACTGTCGCTGCAACCTATGCACCCCGCACCGCTCCCTGGTTTGCAATTCG
CAGCTGCTTAACGAAAGTCAAATTATCGGTACCTTTGAGCTGCAGGGTCCCTCGCCTGACGAAAAGTCCG
CGGCTCCGGGGTTGAAACTCACTCCGGGGCTGTGGACGTCGGCTTACCTTCGCAAATTTGTACCTGAGGA
CTACCACGCCCACGAGATTAGGTTCTACGAAGACCAATCCCGCCCGCCAAATGCGGAGCTTACCGCCTGC
GTCATTACCCAGGGCCACATTCTTGGCCAATTGCAAGCCATCAACAAAGCCCGCCAAGAGTTTCTGCTAC
GAAAGGGACGGGGGGTTTACTTGGACCCCCAGTCCGGCGAGGAGCTCAACCCAATCCCCCCGCCGCCGCA
GCCCTATCAGCAGCAGCCGCGGGCCCTTGCTTCCCAGGATGGCACCCAAAAAGAAGCTGCAGCTGCCGCC
GCCACCCACGGACGAGGAGGAATACTGGGACAGTCAGGCAGAGGAGGTTTTGGACGAGGAGGAGGAGGAC
ATGATGGAAGACTGGGAGAGCCTAGACGAGGAAGCTTCCGAGGTCGAAGAGGTGTCAGACGAAACACCGT
CACCCTCGGTCGCATTCCCCTCGCCGGCGCCCCAGAAATCGGCAACCGGTTCCAGCATGGCTACAACCTC
CGCTCCTCAGGCGCCGCCGGCACTGCCCGTTCGCCGACCCAACCGTAGATGGGACACCACTGGAACCAGG
GCCGGTAAGTCCAAGCAGCCGCCGCCGTTAGCCCAAGAGCAACAACAGCGCCAAGGCTACCGCTCATGGC
GCGGGCACAAGAACGCCATAGTTGCTTGCTTGCAAGACTGTGGGGGCAACATCTCCTTCGCCCGCCGCTT
TCTTCTCTACCATCACGGCGTGGCCTTCCCCCGTAACATCCTGCATTACTACCGTCATCTCTACAGCCCA
TACTGCACCGGCGGCAGCGGCAGCGGCAGCAACAGCAGCGGCCACACAGAAGCAAAGGCGACCGGATAGC
AAGACTCTGACAAAGCCCAAGAAATCCACAGCGGCGGCAGCAGCAGGAGGAGGAGCGCTGCGTCTGGCGC
CCAACGAACCCGTATCGACCCGCGAGCTTAGAAACAGGATTTTTCCCACTCTGTATGCTATATTTCAACA
GAGCAGGGGCCAAGAACAAGAGCTGAAAATAAAAAACAGGTCTCTGCGATCCCTCACCCGCAGCTGCCTG
TATCACAAAAGCGAAGATCAGCTTCGGCGCACGCTGGAAGACGCGGAGGCTCTCTTCAGTAAATACTGCG
CGCTGACTCTTAAGGACTAGTTTCGCGCCCTTTCTCAAATTTAAGCGCGAAAACTACGTCATCTCCAGCG
GCCACACCCGGCGCCAGCACCTGTCGTCAGCGCCATTATGAGCAAGGAAATTCCCACGCCCTACATGTGG
AGTTACCAGCCACAAATGGGACTTGCGGCTGGAGCTGCCCAAGACTACTCAACCCGAATAAACTACATGA
GCGCGGGACCCCACATGATATCCCGGGTCAACGGAATCCGCGCCCACCGAAACCGAATTCTCTTGGAACA
GGCGGCTATTACCACCACACCTCGTAATAACCTTAATCCCCGTAGTTGGCCCGCTGCCCTGGTGTACCAG
GAAAGTCCCGCTCCCACCACTGTGGTACTTCCCAGAGACGCCCAGGCCGAAGTTCAGATGACTAACTCAG
GGGCGCAGCTTGCGGGCGGCTTTCGTCACAGGGTGCGGTCGCCCGGGCAGGGTATAACTCACCTGACAAT
CAGAGGGCGAGGTATTCAGCTCAACGACGAGTCGGTGAGCTCCTCGCTTGGTCTCCGTCCGGACGGGACA
TTTCAGATCGGCGGCGCCGGCCGTCCTTCATTCACGCCTCGTCAGGCAATCCTAACTCTGCAGACCTCGT
CCTCTGAGCCGCGCTCTGGAGGCATTGGAACTCTGCAATTTATTGAGGAGTTTGTGCCATCGGTCTACTT
TAACCCCTTCTCGGGACCTCCCGGCCACTATCCGGATCAATTTATTCCTAACTTTGACGCGGTAAAGGAC
TCGGCGGACGGCTACGACTGAATGTTAAGTGGAGAGGCAGAGCAACTGCGCCTGAAACACCTGGTCCACT
GTCGCCGCCACAAGTGCTTTGCCCGCGACTCCGGTGAGTTTTGCTACTTTGAATTGCCCGAGGATCATAT
CGAGGGCCCGGCGCACGGCGTCCGGCTTACCGCCCAGGGAGAGCTTGCCCGTAGCCTGATTCGGGAGTTT
ACCCAGCGCCCCCTGCTAGTTGAGCGGGACAGGGGACCCTGTGTTCTCACTGTGATTTGCAACTGTCCTA
ACCTTGGATTACATCAAGATCTTTGTTGCCATCTCTGTGCTGAGTATAATAAATACAGAAATTAAAATAT
ACTGGGGCTCCTATCGCCATCCTGTAAACGCCACCGTCTTCACCCGCCCAAGCAAACCAAGGCGAACCTT
ACCTGGTACTTTTAACATCTCTCCCTCTGTGATTTACAACAGTTTCAACCCAGACGGAGTGAGTCTACGA
GAGAACCTCTCCGAGCTCAGCTACTCCATCAGAAAAAACACCACCCTCCTTACCTGCCGGGPACGTACGA
GTGCGTCACCGGCCGCTGCACCACACCTACCGCCTGACCGTAAACCAGACTTTTTCCGGACAGACCTCAA
TAACTCTGTTTACCAGAACAGGAGGTGAGCTTAGAAAACCCTTAGGGTATTAGGCCAAAGGCGCAGCTAC
TGTGGGGTTTATGAACAATTCAAGCAACTCTACGGGCTATTCTAATTCAGGTTTCTCTAGAAATGGACGG
AATTATTACAGAGCAGCGCCTGCTAGAAAGACGCAGGGCAGCGGCCGAGCAACAGCGCATGAATCAAGAG
CTCCAAGACATGGTTAACTTGCACCAGTGCAAAAGGGGTATCTTTTGTCTGGTAAAGCAGGCCAAAGTCA
CCTACGACAGTAATACCACCGGACACCGCCTTAGCTACAAGTTGCCAACCAAGCGTCAGAAATTGGTGGT
CATGGTGGGAGAAAAGCCCATTACCATAACTCAGCACTCGGTAGAAACCGAAGGCTGCATTCACTCACCT
TGTCAAGGACCTGAGGATCTCTGCACCCTTATTAAGACCCTGTGCGGTCTCAAAGATCTTATTCCCTTTA
ACTAATAAAAAAAAATAATAAAGCATCACTTACTTAAAATCAGTTAGCAAATTTCTGTCCAGTTTATTCA
GCAGCACCTCCTTGCCCTCCTCCCAGCTCTGGTATTGCAGCTTCCTCCTGGCTGCAAACTTTCTCCACAA
TCTAAATGGAATGTCAGTTTCCTCCTGTTCCTGTCCATCCGCACCCACTATCTTCATGTTGTTGCAGATG
AAGCGCGCAAGACCGTCTGAAGATACCTTCAACCCCGTGTATCCATATGACACGGAAACCGGTCCTCCAA
CTGTGCCTTTTCTTACTCCTCCCTTTGTATCCCCCAATGGGTTTCAAGAGAGTCCCCCTGGGGTACTCTC
TTTGCGCCTATCCGAACCTCTAGTTACCTCCAATGGCATGCTTGCGCTCAAAATGGGCAACGGCCTCTCT
CTGGACGAGGCCGGCAACCTTACCTCCCAAAATGTAACCACTGTGAGCCCACCTCTCAAAAAAACCAAGT
CAAACATAAACCTGGAAATATCTGCACCCCTCACAGTTACCTCAGAAGCCCTAACTGTGGCTGCCGCCGC
ACCTCTAATGGTCGCGGGCAACACACTCACCATGCAATCACAGGCCCCGCTAACCGTGCACGACTCCAAA
CTTAGCATTGCCACCCAAGGACCCCTCACAGTGTCAGAAGGAAAGCTAGCCCTGCAAACATCAGGCCCCC
TCACCACCACCGATAGCAGTACCCTTACTATCACTGCCTCACCCCCTCTAACTACTGCCACTGGTAGCTT
GGGCATTGACTTGAAAGAGCCCATTTATACACAAAATGGAAAACTAGGACTAAAGTACGGGGCTCCTTTG
CATGTAACAGACGACCTAAACACTTTGACCGTAGCAACTGGTCCAGGTGTGACTATTAATAATACTTCCT
TGCAAACTAAAGTTACTGGAGCCTTGGGTTTTGATTCACAAGGCAATATGCAACTTAATGTAGCAGGAGG
ACTAAGGATTGATTCTCAAAACAGACGCCTTATACTTGATGTTAGTTATCCGTTTGATGCTCAAAACCAA
CTAAATCTAAGACTAGGACAGGGCCCTCTTTTTATAAACTCAGCCCACAACTTGGATATTAACTACAACA
AAGGCCTTTACTTGTTTACAGCTTCAAACAATTCCAAAAAGCTTGAGGTTAACCTAAGCACTGCCAAGGG
GTTGATGTTTGACGCTACAGCCATAGCCATTAATGCAGGAGATGGGCTTGAATTTGGTTCACCTAATGCA
CCAAACACAAATCCCCTCAAAACAAAAATTGGCCATGGCCTAGAATTTGATTCAAACAAGGCTATGGTTC
CTAAACTAGGAACTGGCCTTAGTTTTGACAGCACAGGTGCCATTACAGTAGGAAACAAAAATAATGATAA
GCTAACTTTGTGGACCACACCAGCTCCATCTCCTAACTGTAGACTAAATGCAGAGAAAGATGCTAAACTC
ACTTTGGTCTTAACAAAATGTGGCAGTCAAATACTTGCTACAGTTTCAGTTTTGGCTGTTAAAGGCAGTT
TGGCTCCAATATCTGGAACAGTTCAAAGTGCTCATCTTATTATAAGATTTGACGAAAATGGAGTGCTACT
AAACAATTCCTTCCTGGACCCAGAATATTGGAACTTTAGAAATGGAGATCTTACTGAAGGCACAGCCTAT
ACAAACGCTGTTGGATTTATGCCTAACCTATCAGCTTATCCAAAATCTCACGGTAAAACTGCCAAAAGTA
ACATTGTCAGTCAAGTTTACTTAAACGGAGACAAAACTAAACCTGTAACACTAACCATTACACTAAACGG
TACACAGGAAACAGGAGACACAACTCCAAGTGCATACTCTATGTCATTTTCATGGGACTGGTCTGGCCAC
AACTACATTAATGAAATATTTGCCACATCCTCTTACACTTTTTCATACATTGCCCAAGAATAAAGAATCG
TTTGTGTTATGTTTCAACGTGTTTATTTTTCAATTGCAGAAAATTTCGAATCATTTTTCATTCAGTAGTA
TAGCCCCACCACCACATAGCTTATACAGATCACCGTACCTTAATCAAACTCACAGPACCCTAGTATTCPA
CCTGCCACCTCCCTCCCAACACACAGAGTACACAGTCCTTTCTCCCCGGCTGGCCTTAAAAAGCATCATA
TCATGGGTAACAGACATATTCTTAGGTGTTATATTCCACACGGTTTCCTGTCGAGCCAAACGCTCATCAG
TGATATTAATAAACTCCCCGGGCAGCTCACTTAAGTTCATGTCGCTGTCCAGCTGCTGAGCCACAGGCTG
CTGTCCAACTTGCGGTTGCTTAACGGGCGGCGAAGGAGAAGTCCACGCCTACATGGGGGTAGAGTCATAA
TCGTGCATCAGGATAGGGCGGTGGTGCTGCAGCAGCGCGCGAATAAACTGCTGCCGCCGCCGCTCCGTCC
TGCAGGAATACAACATGGCAGTGGTCTCCTCAGCGATGATTCGCACCGCCCGCAGCATAAGGCGCCTTGT
CCTCCGGGCACAGCAGCGCACCCTGATCTCACTTAAATCAGCACAGTAACTGCAGCACAGCACCACAATA
TTGTTCAAAATCCCACAGTGCAAGGCGCTGTATCCAAAGCTCATGGCGGGGACCACAGAACCCACGTGGC
CATCATACCACAAGCGCAGGTAGATTAAGTGGCGACCCCTCATAAACACGCTGGACATAAACATTACCTC
TTTTGGCATGTTGTAATTCACCACCTCCCGGTACCATATAAACCTCTGATTAAACATGGCGCCATCCACC
ACCATCCTAAACCAGCTGGCCAAAACCTGCCCGCCGGCTATACACTGCAGGGAACCGGGACTGGAACAAT
GACAGTGGAGAGCCCAGGACTCGTAACCATGGATCATCATGCTCGTCATGATATCAATGTTGGCACAACA
CAGGCACACGTGCATACACTTCCTCAGGATTACAAGCTCCTCCCGCGTTAGAACCATATCCCAGGGAACA
ACCCATTCCTGAATCAGCGTAAATCCCACACTGCAGGGAAGACCTCGCACGTAACTCACGTTGTGCATTG
TCAAAGTGTTACATTCGGGCAGCAGCGGATGATCCTCCAGTATGGTAGCGCGGGTTTCTGTCTCAAAAGG
AGGTAGACGATCCCTACTGTACGGAGTGCGCCGAGACAACCGAGATCGTGTTGGTCGTAGTGTCATGCCA
AATGGAACGCCGGACGTAGTCATATTTCCTGAAGCAAAACCAGGTGCGGGCGTGACAAACAGATCTGCGT
CTCCGGTCTCGCCGCTTAGATCGCTCTGTGTAGTAGTTGTAGTATATCCACTCTCTCAAAGCATCCAGGC
GCCCCCTGGCTTCGGGTTCTATGTAAACTCCTTCATGCGCCGCTGCCCTGATAACATCCACCACCGCAGA
ATAAGCCACACCCAGCCAACCTACACATTCGTTCTGCGAGTCACACACGGGAGGAGCGGGAAGAGCTGGA
AGAACCATGTTTTTTTTTTTATTCCAAAAGATTATCCAAAACCTCAAAATGAAGATCTATTAAGTGAACG
CGCTCCCCTCCGGTGGCGTGGTCAAACTCTACAGCCAAAGAACAGATAATGGCATTTGTAAGATGTTGCA
CAATGGCTTCCAAAAGGCAAACGGCCCTCACGTCCAAGTGGACGTAAAGGCTAAACCCTTCAGGGTGAAT
CTCCTCTATAAACATTCCAGCACCTTCAACCATGCCCAAATAATTCTCATCTCGCCACCTTCTCAATATA
TCTCTAAGCAAATCCCGAATATTAAGTCCGGCCATTGTAAAAATCTGCTCCAGAGCGCCCTCCACCTTCA
GCCTCAAGCAGCGAATCATGATTGCAAAAATTCAGGTTCCTCACAGACCTGTATAAGATTCAAAAGCGGA
ACATTAACAAAAATACCGCGATCCCGTAGGTCCCTTCGCAGGGCCAGCTGAACATAATCGTGCAGGTCTG
CACGGACCAGCGCGGCCACTTCCCCGCCAGGAACCTTGACAAAAGAACCCACACTGATTATGACACGCAT
ACTCGGAGCTATGCTAACCAGCGTAGCCCCGATGTAAGCTTTGTTGCATGGGCGGCGATATAAAATGCAA
GGTGCTGCTCAAAAAATCAGGCAPAGCCTCGCGCAAAAAAGAAAGCACATCGTAGTCATGCTCATGCAGA
TAAAGGCAGGTAAGCTCCGGAACCACCACAGAAAAAGACACCATTTTTCTCTCAAACATGTCTGCGGGTT
TCTGCATAAACACAAAATAAAATAACAAAAAAACATTTAAACATTAGAAGCCTGTCTTACAACAGGAAAA
ACAACCCTTATAAGCATAAGACGGACTACGGCCATGCCGGCGTGACCGTAAAAAAACTGGTCACCGTGAT
TAAAAAGCACCACCGACAGCTCCTCGGTCATGTCCGGAGTCATAATGTAAGACTCGGTAAACACATCAGG
TTGATTCACATCGGTCAGTGCTAAAAAGCGACCGAAATAGCCCGGGGGAATACATACCCGCAGGCGTAGA
GACAACATTACAGCCCCCATAGGAGGTATAACAAAATTAATAGGAGAGAAAAACACATAAACACCTGAAA
AACCCTCCTGCCTAGGCAAAATAGCACCCTCCCGCTCCAGAACAACATACAGCGCTTCCACAGCGGCAGC
CATAACAGTCAGCCTTACCAGTAAAAAAGAAAACCTATTAAAAAAACACCACTCGACACGGCACCAGCTC
AATCAGTCACAGTGTAAAAAAGGGCCAAGTGCAGAGCGAGTATATATAGGACTAAAAAATGACGTAACGG
TTAAAGTCCACAAAAAACACCCAGAAAACCGCACGCGAACCTACGCCCAGAAACGAAAGCCAAAAAACCC
ACAACTTCCTCAAATCGTCACTTCCGTTTTCCCACGTTACGTCACTTCCCATTTTAAGAAAACTACAATT
CCCAACACATACAAGTTACTCCGCCCTAAAACCTACGTCACCCGCCCCGTTCCCACGCCCCGCGCCACGT
CACAAACTCCACCCCCTCATTATCATATTGGCTTCAATCCAAAATAAGGTATATTATTGATGATGTTAAT
TAATTTAAATCCGCATGCGATATCGAGCTCTCCCGGGAATTCGGATCTGCGACGCGAGGCTGGATGGCCT
TCCCCATTATGATTCTTCTCGCTTCCGGCGGCATCGGGATGCCCGCGTTGCAGGCCATGCTGTCCAGGCA
GGTAGATGACGACCATCAGGGACAGCTTCACGGCCAGCAAAAGGCCAGGAACCGTAAAAAGGCCGCGTTG
CTGGCGTTTTTCCATAGGCTCCGCCCCCCTGACGAGCATCACAAAAATCGACGCTCAAGTCAGAGGTGGC
GAAACCCGACAGGACTATAPAGATACCAGGCGTTTCCCCCTGGAAGCTCCCTCGTGCGCTCTCCTGTTCC
GACCCTGCCGCTTACCGGATACCTGTCCGCCTTTCTCCCTTCGGGAAGCGTGGCGCTTTCTCAATGCTCA
CGCTGTAGGTATCTCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTACAGAGTTCTTGAAG
TGGTGGCCTAACTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTGAC
GCTCAGTGGAACGAAAACTCACGTTAAGGGATTTTGGTCATGAGATTATCAAAAAGGATCTTCACCTAGA
TCCTTTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGATAACTA
CGATACGGGAGGGCTTACCATCTGGCCCCAGTGCTGCAATGATACCGCGAGACCCACGCTCACCGGCTCC
AGATTTATCAGCAATAAACCAGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCCAGTTAATAGTTTGCGCAACG
TTGTTGCCATTGNTGCAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTG
TATGCGGCGACCGAGTTGCTCTTGCCCGGCGTCAACACGGGATAATACCGCGCCACATAGCAGAACTTTA
AAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGAAATGTTGAATACTCATA
CTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTCTTCAAGGATCC
GAATTCCCGGGAGAGCTCGATATCGCATGCGGATTTAAATTAATTAA

TABLE 12
Nucleotide sequence of pIB/V5-His-DEST.

OpIE-2 pr

| | | | | | |
|---|---|---|---|---|---|
| 1 | CATGATGATA | AACAATGTAT | GGTGCTAATG | TTGCTTCAAC | AACAATTCTG |
| | GTACTACTAT | TTGTTACATA | CCACGATTAC | AACGAAGTTG | TTGTTAAGAC |
| 51 | TTGAACTGTG | TTTTCATGTT | TGCCAACAAG | CACCTTTATA | CTCGGTGGCC |
| | AACTTGACAC | AAAAGTACAA | ACGGTTGTTC | GTGGAAATAT | GAGCCACCGG |
| 101 | TCCCCACCAC | CAACTTTTTT | GCACTGCAAA | AAAACACGCT | TTTGCACGCG |
| | AGGGGTGGTG | GTTGAAAAAA | CGTGACGTTT | TTTTGTGCGA | AAACGTGCGC |
| 151 | GGCCCATACA | TAGTACAAAC | TCTACGTTTC | GTAGACTATT | TTACATAAAT |
| | CCGGGTATGT | ATCATGTTTG | AGATGCAAAG | CATCTGATAA | AATGTATTTA |
| 201 | AGTCTACACC | GTTGTATACG | CTCCAAATAC | ACTACCACAC | ATTGAACCTT |
| | TCAGATGTGG | CAACATATGC | GAGGTTTATG | TGATGGTGTG | TAACTTGGAA |
| 251 | TTTGCAGTGC | AAAAAAGTAC | GTGTCGGCAG | TCACGTAGGC | CGGCCTTATC |
| | AAACGTCACG | TTTTTTCATG | CACAGCCGTC | AGTGCATCCG | GCCGGAATAG |
| 301 | GGGTCGCGTC | CTGTCACGTA | CGAATCACAT | TATCGGACCG | GACGAGTGTT |
| | CCCAGCGCAG | GACAGTGCAT | GCTTAGTGTA | ATAGCCTGGC | CTGCTCACAA |
| 351 | GTCTTATCGT | GACAGGACGC | CAGCTTCCTG | TGTTGCTAAC | CGCAGCCGGA |
| | CAGAATAGCA | CTGTCCTGCG | GTCGAAGGAC | ACAACGATTG | GCGTCGGCCT |
| 401 | CGCAACTCCT | TATCGGAACA | GGACGCGCCT | CCATATCAGC | CGCGCGTTAT |
| | GCGTTGAGGA | ATAGCCTTGT | CCTGCGCGGA | GGTATAGTCG | GCGCGCAATA |
| 451 | CTCATGCACG | TGACCGGACA | CGAGGCGCCC | GTCCCGCTTA | TCGCGCCTAT |
| | GAGTACGTGC | ACTGGCCTGT | GCTCCGCGGG | CAGGGCGAAT | AGCGCGGATA |

OpIE2FOR
OpIE-2 pr

| | | | | | |
|---|---|---|---|---|---|
| 501 | AAATACAGCC | CGCAACGATC | TGGTAAACAC | AGTTGAACAG | CATCTGTTCG |
| | TTTATGTCGG | GCGTTGCTAG | ACCATTTGTG | TCAACTTGTC | GTAGACAAGC |
| 551 | AATTTAAAGC | TTGATATCGA | ATTCCTGCAG | CCCAGCGCTG | GATCCTCGAT |
| | TTAAATTTCG | AACTATAGCT | TAAGGACGTC | GGGTCGCGAC | CTAGGAGCTA |

attR1

| | | | | | |
|---|---|---|---|---|---|
| 601 | CACAAGTTTG | TACAAAAAAG | CTGAACGAGA | AACGTAAAAT | GATATAAATA |
| | GTGTTCAAAC | ATGTTTTTTC | GACTTGCTCT | TTGCATTTTA | CTATATTTAT |
| 651 | TCAATATATT | AAATTAGATT | TTGCATAAAA | AACAGACTAC | ATAATACTGT |
| | AGTTATATAA | TTTAATCTAA | AACGTATTTT | TTGTCTGATG | TATTATGACA |
| 701 | AAAACACAAC | ATATCCAGTC | ACTATGGCGG | CCGCATTAGG | CACCCCAGGC |
| | TTTTGTGTTG | TATAGGTCAG | TGATACCGCC | GGCGTAATCC | GTGGGGTCCG |
| 751 | TTTACACTTT | ATGCTTCCGG | CTCGTATAAT | GTGTGGATTT | TGAGTTAGGA |
| | AAATGTGAAA | TACGAAGGCC | GAGCATATTA | CACACCTAAA | ACTCAATCCT |

Cmr

| | | | | | |
|---|---|---|---|---|---|
| 801 | TCCGTCGAGA | TTTTCAGGAG | CTAAGGAAGC | TAAAATGGAG | AAAAAAATCA |
| | AGGCAGCTCT | AAAAGTCCTC | GATTCCTTCG | ATTTTACCTC | TTTTTTTAGT |
| 851 | CTGGATATAC | CACCGTTGAT | ATATCCCAAT | GGCATCGTAA | AGAACATTTT |
| | GACCTATATG | GTGGCAACTA | TATAGGGTTA | CCGTAGCATT | TCTTGTAAAA |
| 901 | GAGGCATTTC | AGTCAGTTGC | TCAATGTACC | TATAACCAGA | CCGTTCAGCT |
| | CTCCGTAAAG | TCAGTCAACG | AGTTACATGG | ATATTGGTCT | GGCAAGTCGA |
| 951 | GGATATTACG | GCCTTTTTAA | AGACCGTAAA | GAAAAATAAG | CACAAGTTTT |
| | CCTATAATGC | CGGAAAAATT | TCTGGCATTT | CTTTTTATTC | GTGTTCAAAA |
| 1001 | ATCCGGCCTT | TATTCACATT | CTTGCCCGCC | TGATGAATGC | TCATCCGGAA |
| | TAGGCCGGAA | ATAAGTGTAA | GAACGGGCGG | ACTACTTACG | AGTAGGCCTT |
| 1051 | TTCCGTATGG | CAATGAAAGA | CGGTGAGCTG | GTGATATGGG | ATAGTGTTCA |
| | AAGGCATACC | GTTACTTTCT | GCCACTCGAC | CACTATACCC | TATCACAAGT |
| 1101 | CCCTTGTTAC | ACCGTTTTCC | ATGAGCAAAC | TGAAACGTTT | TCATCGCTCT |
| | GGGAACAATG | TGGCAAAAGG | TACTCGTTTG | ACTTTGCAAA | AGTAGCGAGA |
| 1151 | GGAGTGAATA | CCACGACGAT | TTCCGGCAGT | TTCTACACAT | ATATTCGCAA |
| | CCTCACTTAT | GGTGCTGCTA | AAGGCCGTCA | AAGATGTGTA | TATAAGCGTT |
| 1201 | GATGTGGCGT | GTTACGGTGA | AAACCTGGCC | TATTTCCCTA | AAGGGTTTAT |
| | CTACACCGCA | CAATGCCACT | TTTGGACCGG | ATAAAGGGAT | TTCCCAAATA |
| 1251 | TGAGAATATG | TTTTTCGTCT | CAGCCAATCC | CTGGGTGAGT | TTCACCAGTT |
| | ACTCTTATAC | AAAAAGCAGA | GTCGGTTAGG | GACCCACTCA | AAGTGGTCAA |
| 1301 | TTGATTTAAA | CGTGGCCAAT | ATGGACAACT | TCTTCGCCCC | CGTTTTCACC |
| | AACTAAATTT | GCACCGGTTA | TACCTGTTGA | AGAAGCGGGG | GCAAAAGTGG |
| 1351 | ATGGGCAAAT | ATTATACGCA | AGGCGACAAG | GTGCTGATGC | CGCTGGCGAT |
| | TACCCGTTTA | TAATATGCGT | TCCGCTGTTC | CACGACTACG | GCGACCGCTA |
| 1401 | TCAGGTTCAT | CATGCCGTTT | GTGATGGCTT | CCATGTCGGC | AGAATGCTTA |
| | AGTCCAAGTA | GTACGGCAAA | CACTACCGAA | GGTACAGCCG | TCTTACGAAT |
| 1451 | ATGAATTACA | ACAGTACTGC | GATGAGTGGC | AGGGCGGGGC | GTAAACGCGT |
| | TACTTAATGT | TGTCATGACG | CTACTCACCG | TCCCGCCCCG | CATTTGCGCA |
| 1501 | GGATCCGGCT | TACTAAAAGC | CAGATAACAG | TATGCGTATT | TGCGCGCTGA |
| | CCTAGGCCGA | ATGATTTTCG | GTCTATTGTC | ATACGCATAA | ACGCGCGACT |
| 1551 | TTTTTGCGGT | ATAAGAATAT | ATACTGATAT | GTATACCCGA | AGTATGTCAA |
| | AAAAACGCCA | TATTCTTATA | TATGACTATA | CATATGGGCT | TCATACAGTT |
| 1601 | AAAGAGGTAT | GCTATGAAGC | AGCGTATTAC | AGTGACAGTT | GACAGCGACA |
| | TTTCTCCATA | CGATACTTCG | TCGCATAATG | TCACTGTCAA | CTGTCGCTGT |
| 1651 | GCTATCAGTT | GCTCAAGGCA | TATATGATGT | CAATATCTCC | GGTCTGGTAA |
| | CGATAGTCAA | CGAGTTCCGT | ATATACTACA | GTTATAGAGG | CCAGACCATT |
| 1701 | GCACAACCAT | GCAGAATGAA | GCCCGTCGTC | TGCGTGCCGA | ACGCTGGAAA |
| | CGTGTTGGTA | CGTCTTACTT | CGGGCAGCAG | ACGCACGGCT | TGCGACCTTT |
| 1751 | GCGGAAAATC | AGGAAGGGAT | GGCTGAGGTC | GCCCGGTTTA | TTGAAATGAA |
| | CGCCTTTTAG | TCCTTCCCTA | CCGACTCCAG | CGGGCCAAAT | AACTTTACTT |

ccdB

| | | | | | |
|---|---|---|---|---|---|
| 1801 | CGGCTCTTTT | GCTGACGAGA | ACAGGGGCTG | GTGAAATGCA | GTTTAAGGTT |
| | GCCGAGAAAA | CGACTGCTCT | TGTCCCCGAC | CACTTTACGT | CAAATTCCAA |
| 1851 | TACACCTATA | AAAGAGAGAG | CCGTTATCGT | CTGTTTGTGG | ATGTACAGAG |
| | ATGTGGATAT | TTTCTCTCTC | GGCAATAGCA | GACAAACACC | TACATGTCTC |
| 1901 | TGATATTATT | GACACGCCCG | GGCGACGGAT | GGTGATCCCC | CTGGCCAGTG |
| | ACTATAATAA | CTGTGCGGGC | CCGCTGCCTA | CCACTAGGGG | GACCGGTCAC |
| 1951 | CACGTCTGCT | GTCAGATAAA | GTCTCCCGTG | AACTTTACCC | GGTGGTGCAT |
| | GTGCAGACGA | CAGTCTATTT | CAGAGGGCAC | TTGAAATGGG | CCACCACGTA |
| 2001 | ATCGGGGATG | AAAGCTGGCG | CATGATGACC | ACCGATATGG | CCAGTGTGCC |
| | TAGCCCCTAC | TTTCGACCGC | GTACTACTGG | TGGCTATACC | GGTCACACGG |
| 2051 | GGTCTCCGTT | ATCGGGGAAG | AAGTGGCTGA | TCTCAGCCAC | CGCGAAAATG |
| | CCAGAGGCAA | TAGCCCCTTC | TTCACCGACT | AGAGTCGGTG | GCGCTTTTAC |
| 2101 | ACATCAAAAA | CGCCATTAAC | CTGATGTTCT | GGGGAATATA | AATGTCAGGC |
| | TGTAGTTTTT | GCGGTAATTG | GACTACAAGA | CCCCTTATAT | TTACAGTCCG |

attR2

| | | | | | |
|---|---|---|---|---|---|
| 2151 | TCCCTTATAC | ACAGCCAGTC | TGCAGGTCGA | CCATAGTGAC | TGGATATGTT |
| | AGGGAATATG | TGTCGGTCAG | ACGTCCAGCT | GGTATCACTG | ACCTATACAA |
| 2201 | GTGTTTTACA | GTATTATGTA | GTCTGTTTTT | TATGCAAAAT | CTAATTTAAT |
| | CACAAAATGT | CATAATACAT | CAGACAAAAA | ATACGTTTTA | GATTAAATTA |
| 2251 | ATATTGATAT | TTATATCATT | TTACGTTTCT | CGTTCAGCTT | TCTTGTACAA |
| | TATAACTATA | AATATAGTAA | AATGCAAAGA | GCAAGTCGAA | AGAACATGTT |

V5 tag

| | | | | | |
|---|---|---|---|---|---|
| 2301 | AGTGGTGATC | GACCCGGGTC | TAGAGGGCCC | GCGGTTCGAA | GGTAAGCCTA |
| | TCACCACTAG | CTGGGCCCAG | ATCTCCCGGG | CGCCAAGCTT | CCATTCGGAT |

Poly His 6 tag

| | | | | | |
|---|---|---|---|---|---|
| 2351 | TCCCTAACCC | TCTCCTCGGT | CTCGATTCTA | CGCGTACCGG | TCATCATCAC |
| | AGGGATTGGG | AGAGGAGCCA | GAGCTAAGAT | GCGCATGGCC | AGTAGTAGTG |

OpIE-2 PolyA

| | | | | | |
|---|---|---|---|---|---|
| 2401 | CATCACCATT | GAGTTTATCT | GACTAAATCT | TAGTTTGTAT | TGTCATGTTT |
| | GTAGTGGTAA | CTCAAATAGA | CTCATTTAGA | ATCAAACATA | ACAGTACAAA |
| 2451 | TAATACAATA | TGTTATGTTT | AAATATGTTT | TTAATAAATT | TTATAAAATA |
| | ATTATGTTAT | ACAATACAAA | TTTATACAAA | AATTATTTAA | AATATTTTAT |
| 2501 | ATTTCAACTT | TTATTGTAAC | AACATTGTCC | ATTTACACAC | TCCTTTCAAG |
| | TAAAGTTGAA | AATAACATTG | TTGTAACAGG | TAAATGTGTG | AGGAAAGTTC |
| 2551 | CGCGTGGGAT | CGATGCTCAC | TCAAAGGCGG | TAATACGGTT | ATCCACAGAA |
| | GCGCACCCTA | GCTACCAGTG | AGTTTCCGCC | ATTATGCCAA | TAGGTGTCTT |

pMB1 ori

| | | | | | |
|---|---|---|---|---|---|
| 2601 | TCAGGGGATA | ACGCAGGAAA | GAACATGTGA | GCAAAAGGCC | AGCAAAAGGC |
| | AGTCCCCTAT | TGCGTCCTTT | CTTGTACACT | CGTTTTCCGG | TCGTTTTCCG |
| 2651 | CAGGAACCGT | AAAAAGGCCG | CGTTGCTGGC | GTTTTTCCAT | AGGCTCCGCC |
| | GTCCTTGGCA | TTTTTCCGGC | GCAACGACCG | CAAAAAGGTA | TCCGAGGCGG |
| 2701 | CCCCTGACGA | GCATCACATA | AATCGACGCT | CAAGTCAGAG | GTGGCGAAAC |
| | GGGGACTGCT | CGTAGTGTTT | TTAGCTGCGA | GTTCAGTCTC | CACCGCTTTG |
| 2751 | CCGACAGGAC | TATPAAGATA | CCAGGCGTTT | CCCCCTGGAA | GCTCCCTCGT |
| | GGCTGTCCTG | ATATTTCTAT | GGTCCGCAAA | GGGGGACCTT | CGAGGGAGCA |
| 2801 | GCGCTCTCCT | GTTCCGACCC | TGCCGCTTAC | CGGATACCTG | TCCGCCTTTC |
| | CGCGAGAGGA | CAAGGCTGGG | ACGGCGAATG | GCCTATGGAC | AGGCGGAAAG |
| 2851 | TCCCTTCGGG | AAGCGTGGCG | CTTTCTCATA | GCTCACGCTG | TAGGTATCTC |
| | AGGGAAGCCC | TTCGCACCGC | GAAAGAGTAT | CGAGTGCGAC | ATCCATAGAG |
| 2901 | AGTTCGGTGT | AGGTCGTTCG | CTCCAAGCTG | GGCTGTGTGC | ACGAACCCCC |
| | TCAAGCCACA | TCCAGCAAGC | GAGGTTCGAC | CCGACACACG | TGCTTGGGGG |
| 2951 | CGTTCAGCCC | GACCGCTGCG | CCTTATCCGG | TAACTATCGT | CTTGAGTCCA |
| | GCAAGTCGGG | CTGGCGACGC | GGAATAGGCC | ATTGATAGCA | GAACTCAGGT |
| 3001 | ACCCGGTAAG | ACACGACTTA | TCGCCACTGG | CAGCAGCCAC | TGGTAACAGG |
| | TGGGCCATTC | TGTGCTGAAT | AGCGGTGACC | GTCGTCGGTG | ACCATTGTCC |
| 3051 | ATTAGCAGAG | CGAGGTATGT | AGGCGGTGCT | ACAGAGTTCT | TGAAGTGGTG |
| | TAATCGTCTC | GCTCCATACA | TCCGCCACGA | TGTCTCAAGA | ACTTCACCAC |
| 3101 | GCCTAACTAC | GGCTACACTA | GAAGAACAGT | ATTTGGTATC | TGCGCTCTGC |
| | CGGATTGATG | CCGATGTGAT | CTTCTTGTCA | TAAACCATAG | ACGCGAGACG |
| 3151 | TGAAGCCAGT | TACCTTCGGA | AAAAGAGTTG | GTAGCTCTTG | ATCCGGCAAA |
| | ACTTCGGTCA | ATGGAAGCCT | TTTTCTCAAC | CATCGAGAAC | TAGGCCGTTT |
| 3201 | CAAACCACCG | CTGGTAGCGG | TGGTTTTTTT | GTTTGCAAGC | AGCAGATTAC |
| | GTTTGGTGGC | GACCATCGCC | ACCAAAAAAA | CAAACGTTCG | TCGTCTAATG |
| 3251 | GCGCAGAAAA | AAAGGATCTC | AAGAAGATCC | TTTGATCTTT | TCTACGGGGT |
| | CGCGTCTTTT | TTTCCTAGAG | TTCTTCTAGG | AAACTAGAAA | AGATGCCCCA |
| 3301 | CTGACGCTCA | GTGGAACGAA | AACTCACGTT | AAGGGATTTT | GGTCATGCCC |
| | GACTGCGAGT | CACCTTGCTT | TTGAGTGCAA | TTCCCTAAAA | CCAGTACGGG |

GP64 promoter

| | | | | | |
|---|---|---|---|---|---|
| 3351 | TTGTTCCGAA | GGGTTGTGTC | ACGTAGGCCA | GATAACGGTC | GGGTATATAA |
| | AACAAGGCTT | CCCAACACAG | TGCATCCGGT | CTATTGCCAG | CCCATATATT |
| 3401 | GATGCCTCAA | TGCTACTAGT | AAATCAGTCA | CACCAAGGCT | TCAATAAGGA |
| | CTACGGAGTT | ACGATGATCA | TTTAGTCAGT | GTGGTTCCGA | AGTTATTCCT |

EM7

| | | | | | |
|---|---|---|---|---|---|
| 3451 | ACACACAAGC | AAGCCCTTTG | AGTCAAGGGC | TGCCGGGCTG | CAGCACGTGT |
| | TGTGTGTTCG | TTCGGGAAAC | TCAGTTCCCG | ACGGCCCGAC | GTCGTGCACA |
| 3501 | TGACAATTAA | TCATCGGCAT | AGTATATCGG | CATAGTATAA | TACGACPAGG |
| | ACTGTTAATT | AGTAGCCGTA | TCATATAGCC | GTATCATATT | ATGCTGTTCC |

Blasticidin (r)

| | | | | | |
|---|---|---|---|---|---|
| 3551 | TGAGGAACTA | AACCATGGCC | AAGCCTTTGT | CTCAAGAAGA | ATCCACCCTC |
| | ACTCCTTGAT | TTGGTACCGG | TTCGGAAACA | GAGTTCTTCT | TAGGTGGGAG |
| 3601 | ATTGAAAGAG | CAACGGCTAC | AATCAACAGC | ATCCCCATCT | CTGAAGACTA |
| | TAACTTTCTC | GTTGCCGATG | TTAGTTGTCG | TAGGGGTAGA | GACTTCTGAT |
| 3651 | CAGCGTCGCC | GGCGCAGCTC | TCTCTAGCGA | CGGCCGCATC | TTCACTGGTG |
| | GTCGCAGCGG | CCGCGTCGAG | AGAGATCGCT | GCCGGCGTAG | AAGTGACCAC |
| 3701 | TCAATGTATA | TCATTTTACT | GGGGGACCTT | GCGCAGAACT | CGTGGTGCTG |
| | AGTTACATAT | AGTAAAATGA | CCCCCTGGAA | CGCGTCTTGA | GCACCACGAC |
| 3751 | GGCACTGCTG | CTGCTGCGGC | AGCTGGCAAC | CTGACTTGTA | TCGTCGCGAT |
| | CCGTGACGAC | GACGACGCCG | TCGACCGTTG | GACTGAACAT | AGCAGCGCTA |
| 3801 | CGGAAATGAG | AACAGGGGCA | TCTTGAGCCC | CTGCGGACGG | TGCCGACAGG |
| | GCCTTTACTC | TTGTCCCCGT | AGAACTCGGG | GACGCCTGCC | ACGGCTGTCC |
| 3851 | TTCTTCTCGA | TCTGCATCCT | GGGATCAAAG | CCATAGTGAA | GGACAGTGAT |
| | AAGAAGAGCT | AGACGTAGGA | CCCTAGTTTC | GGTATCACTT | CCTGTCACTA |
| 3901 | GGACAGCCGA | CGGCAGTTGG | GATTCGTGAA | TTGCTGCCCT | CTGGTTATGT |
| | CCTGTCGGCT | GCCGTCAACC | CTAAGCACTT | AACGACGGGA | GACCAATACA |
| 3951 | GTGGGAGGGC | TAAGCACTTC | GTGGCCGAGG | AGCAGGACTG | ACACGTCCCG |
| | CACCCTCCCG | ATTCGTGAAG | CACCGGCTCC | TCGTCCTGAC | TGTGCAGGGC |
| 4001 | GGAGATCTGC | ATGTCTACTA | AACTCACAAA | TTAGAGCTTC | AATTTAATTA |
| | CCTCTAGACG | TACAGATGAT | TTGAGTGTTT | AATCTCGAAG | TTAAATTAAT |

Amp (r)

| | | | | | |
|---|---|---|---|---|---|
| 4051 | TATCAGTTAT | TACCCATTGA | AAAAGGAAGA | GTATGAGTAT | TCAACATTTC |
| | ATAGTCAATA | ATGGGTAACT | TTTTCCTTCT | CATACTCATA | AGTTGTAAAG |
| 4101 | CGTGTCGCCC | TTATTCCCTT | TTTTGCGGCA | TTTTGCCTTC | CTGTTTTTGC |
| | GCACAGCGGG | AATAAGGGAA | AAAACGCCGT | AAAACGGAAG | GACAAAAACG |
| 4151 | TCACCCAGAA | ACGCTGGTGA | AAGTAAAAGA | TGCTGAAGAT | CACTTGGGTG |
| | AGTGGGTCTT | TGCGACCACT | TTCATTTTCT | ACGACTTCTA | GTCAACCCAC |
| 4201 | CACGAGTGGG | TTACATCGAA | CTGGATCTCA | ACAGCGGTAA | GATCCTTGAG |
| | GTGCTCACCC | AATGTAGCTT | GACCTAGAGT | TGTCGCCATT | CTAGGAACTC |
| 4251 | AGTTTTCGCC | CCGAAGAACG | TTTTCCAATG | ATGAGCACTT | TTAAAGTTCT |
| | TCAAAAGCGG | GGCTTCTTGC | AAAAGGTTAC | TACTCGTGAA | AATTTCAAGA |
| 4301 | GCTATGTGGC | GCGGTATTAT | CCCGTATTGA | CGCCGGGCAA | GAGCAACTCG |
| | CGATACACCG | CGCCATAATA | GGGCATAACT | GCGGCCCGTT | CTCGTTGAGC |
| 4351 | GTCGCCGCAT | ACACTATTCT | CAGAATGACT | TGGTTGAGTA | CTCACCAGTC |
| | CAGCGGCGTA | TGTGATAAGA | GTCTTACTGA | ACCAACTCAT | GAGTGGTCAG |
| 4401 | ACAGAAAAGC | ATCTTACGGA | TGGCATGACA | GTAAGAGAAT | TATGCAGTGC |
| | TGTCTTTTCG | TAGAATGCCT | ACCGTACTGT | CATTCTCTTA | ATACGTCACG |
| 4451 | TGCCATAACC | ATGAGTGATA | ACACTGCGGC | CAACTTACTT | CTGACAACGA |
| | ACGGTATTGG | TACTCACTAT | TGTGACGCCG | GTTGAATGAA | GACTGTTGCT |
| 4501 | TCGGAGGACC | GAAGGAGCTA | ACCGCTTTTT | TGCACAACAT | GGGGGATCAT |
| | AGCCTCCTGG | CTTCCTCGAT | TGGCGAAAAA | ACGTGTTGTA | CCCCCTAGTA |
| 4551 | GTAACTCGCC | TTGATCGTTG | GGAACCGGAG | CTGAATGAAG | CCATACCAAA |
| | CATTGAGCGG | AACTAGCAAC | CCTTGGCCTC | GACTTACTTC | GGTATGGTTT |
| 4601 | CGACGAGCGT | GACACCACGA | TGCCTGTAGC | AATGGCAACA | ACGTTCCGCA |
| | GCTGCTCGCA | CTGTGGTGCT | ACGGACATCG | TTACCGTTGT | TGCAACGCGT |
| 4651 | AACTATTAAC | TGGCGAACTA | CTTACTCTAG | CTTCCCGGCA | ACAATTAATA |
| | TTGATAATTG | ACCGCTTGAT | GAATGAGATC | GAAGGGCCGT | TGTTAATTAT |
| 4701 | GACTGGATGG | AGGCGGATAA | AGTTGCAGGA | CCACTTCTGC | GCTCGGCCCT |
| | CTGACCTACC | TCCGCCTATT | TCAACGTCCT | GGTGAAGACG | CGAGCCGGGA |
| 4751 | TCCGGCTGGC | TGGTTTATTG | CTGATAAATC | TGGAGCCGGT | GAGCGTGGGT |
| | AGGCCGACCG | ACCAAATAAC | GACTATTTAG | ACCTCGGCCA | CTCGCACCCA |
| 4801 | CTCGCGGTAT | CATTGCAGCA | CTGGGGCCAG | ATGGTAAGCC | CTCCCGTATC |
| | GAGCGCCATA | GTAACGTCGT | GACCCCGGTC | TACCATTCGG | GAGGGCATAG |
| 4851 | GTAGTTATCT | ACACGACGGG | GAGTCAGGCA | ACTATGGATG | AACGAAATAG |
| | CATCAATAGA | TGTGCTGCCC | CTCAGTCCGT | TGATACCTAC | TTGCTTTATC |
| 4901 | ACAGATCGCT | GAGATAGGTG | CCTCACTGAT | TAAGCATTGG | TAACTGTCAG |
| | TGTCTAGCGA | CTCTATCCAC | GGAGTGACTA | ATTCGTAACC | ATTGACAGTC |
| 4951 | ACCAAGTTTA | CTCATATATA | CTTTAGATTG | ATTTAAAACT | TCATTTTTAA |
| | TGGTTCAAAT | GAGTATATAT | GAAATCTAAC | TAAATTTTGA | AGTAAAAATT |
| 5001 | TTTAAAAGGA | TCTAGGTGAA | GATCCTTTTT | GATAATCT | |
| | AAATTTTCCT | AGATCCACTT | CTAGGAAAAA | CTATTAGA | |

TABLE 13
Nucleotide sequence of the V5-His DEST cassette.

| | | | | | |
|---|---|---|---|---|---|
| | ph promoter | | | | |
| | ----- | | | | |
| 1 | ATAAGTATTT | TACTGTTTTC | GTAACAGTTT | TGTAATAAAA | AAACCTATAA |
| | TATTCATAAA | ATGACAAAAG | CATTGTCAAA | ACATTATTTT | TTTGGATATT |
| 51 | ATATTCCGGA | TTATTCATAC | CGTCCCACCA | TCGGGCGCGG | ATCCCCGGGT |
| | TATAAGGCCT | AATAAGTATG | GCAGGGTGGT | AGCCCGCGCC | TAGGGGCCCA |

att R1

---------------------------------------------

| | | | | | |
|---|---|---|---|---|---|
| 101 | ACCGATATCA | CAAGTTTGTA | CAAAAAAGCT | GAACGAGAAA | CGTAAAATGA |
| | TGGCTATAGT | GTTCAAACAT | GTTTTTTCGA | CTTGCTCTTT | GCATTTTACT |

att R1

------------------------------------------------------

| | | | | | |
|---|---|---|---|---|---|
| 151 | TATAAATATC | AATATATTAA | ATTAGATTTT | GCATAAAAAA | CAGACTACAT |
| | ATATTTATAG | TTATATAATT | TAATCTAAAA | CGTATTTTTT | GTCTGATGTA |
| | att R1 | | | | |
| | ------- | | | | |
| 201 | AATACTGTAA | AACACAACAT | ATCCAGTCAC | TATGGCGGCC | GCTCCCTAAC |
| | TTATGACATT | TTGTGTTGTA | TAGGTCAGTG | ATACCGCCGG | CGAGGGATTG |
| 251 | CCACGGGGCC | CGTGGCTATG | GCAGGGCTTG | CCGCCCCGAC | GTTGGCTGCG |
| | GGTGCCCCGG | GCACCGATAC | CGTCCCGAAC | GGCGGGGCTG | CAACCGACGC |
| 301 | AGCCCTGGGC | CTTCACCCGA | ACTTGGGGGT | TGGGGTGGGG | AAAAGGAAGA |
| | TCGGGACCCG | GAAGTGGGCT | TGAACCCCCA | ACCCCACCCC | TTTTCCTTCT |
| 351 | AACGCGGGCG | TATTGGTCCC | AATGGGGTCT | CGGTGGGGTA | TCGACAGAGT |
| | TTGCGCCCGC | ATAACCAGGG | TTACCCCAGA | GCCACCCCAT | AGCTGTCTCA |
| 401 | GCCAGCCCTG | GGACCGAACC | CCGCGTTTAT | GAACAAACGA | CCCAACACCC |
| | CGGTCGGGAC | CCTGGCTTGG | GGCGCAAATA | CTTGTTTGCT | GGGTTGTGGG |
| 451 | GTGCGTTTTA | TTCTGTCTTT | TTATTGCCGT | CATAGCGCGG | GTTCCTTCCG |
| | CACGCAAAAT | AAGACAGAAA | AATAACGGCA | GTATCGCGCC | CAAGGAAGGC |
| 501 | GTATTGTCTC | CTTCCGTGTT | TCAGTTAGCC | TCCCCCATCT | CCCGGGCAAA |
| | CATAACAGAG | GAAGGCACAA | AGTCAATCGG | AGGGGGTAGA | GGGCCCGTTT |

---------------------------

tk gene

N A E G M E R A F

| | | | | | |
|---|---|---|---|---|---|
| 551 | CGTGCGCGCC | AGGTCGCAGA | TCGTCGGTAT | GGAGCCTGGG | GTGGTGACGT |
| | GCACGCGCGG | TCCAGCGTCT | AGCAGCCATA | CCTCGGACCC | CACCACTGCA |

------------------------------------------------------

tk gene

T R A L D C I T P I S G P T T V H .

| | | | | | |
|---|---|---|---|---|---|
| 601 | GGGTCTGGAC | CATCCCGGAG | GTAAGTTGCA | GCAGGGCGTC | CCGGCAGCCG |
| | CCCAGACCTG | GTAGGGCCTC | CATTCAACGT | CGTCCCGCAG | GGCCGTCGGC |

------------------------------------------------------

tk gene

. T Q V M G S T L Q L L A D R C G A .

| | | | | | |
|---|---|---|---|---|---|
| 651 | GCGGGCGATT | GGTCGTAATC | CAGGATAAAG | ACATGCATGG | GACGGAGGCG |
| | CGCCCGCTAA | CCAGCATTAG | GTCCTATTTC | TGTACGTACC | CTGCCTCCGC |

------------------------------------------------------

tk gene

.. P S Q D Y D L I F V H M P R L R

| | | | | | |
|---|---|---|---|---|---|
| 701 | TTTGGCCAAG | ACGTCCAAAG | CCCAGGCAAA | CACGTTATAC | AGGTCGCCGT |
| | AAACCGGTTC | TGCAGGTTTC | GGGTCCGTTT | GTGCAATATG | TCCAGCGGCA |

------------------------------------------------------

tk gene

K A L V D L A W A F V N Y L D G N .

| | | | | | |
|---|---|---|---|---|---|
| 751 | TGGGGGCCAG | CAACTCGGGG | GCCCGAAACA | GGGTAAATAA | CGTGTCCCCG |
| | ACCCCCGGTC | GTTGAGCCCC | CGGGCTTTGT | CCCATTTATT | GCACAGGGGC |

------------------------------------------------------

tk gene

. P A L L E P A R F L T F L T D G I .

| | | | | | |
|---|---|---|---|---|---|
| 801 | ATATGGGGTC | GTGGGCCCGC | GTTGCTCTGG | GGCTCGGCAC | CCTGGGGCGG |
| | TATACCCCAG | CACCCGGGCG | CAACGAGACC | CCGAGCCGTG | GGACCCCGCC |

------------------------------------------------------

tk gene

.. H P R P G A N S Q P E A G Q P P

| | | | | | |
|---|---|---|---|---|---|
| 851 | CACGGCCGCC | CCCGAAAGCT | GTCCCCAATC | CTCCCGCCAC | GACCCGCCGC |
| | GTGCCGGCGG | GGGCTTTCGA | CAGGGGTTAG | GAGGGCGGTG | CTGGGCGGCG |

------------------------------------------------------

tk gene

V A A G S L Q G W D E R W S G G G .

| | | | | | |
|---|---|---|---|---|---|
| 901 | CCTGCAGATA | CCGCACCGTA | TTGGCAAGCA | GCCCATAAAC | GCGGCGAATC |
| | GGACGTCTAT | GGCGTGGCAT | AACCGTTCGT | CGGGTATTTG | CGCCGCTTAG |

------------------------------------------------------

tk gene

. Q L Y R V T N A L L G Y V R R I A .

| | | | | | |
|---|---|---|---|---|---|
| 951 | GCGGCCAGCA | TAGCCAGGTC | AAGCCGCTCG | CCGGGGCGCT | GGCGTTTGGC |
| | CGCCGGTCGT | ATCGGTCCAG | TTCGGCGAGC | GGCCCCGCGA | CCGCAAACCG |

------------------------------------------------------

tk gene

.. A L M A L D L R E G P R Q R K A

| | | | | | |
|---|---|---|---|---|---|
| 1001 | CAGGCGGTCG | ATGTGTCTGT | CCTCCGGAAG | GGCCCCCAAC | ACGATGTTTG |
| | GTCCGCCAGC | TACACAGACA | GGAGGCCTTC | CCGGGGGTTG | TGCTACAAAC |

------------------------------------------------------

tk gene

L R D I H R D E P L A G L V I N T .

| | | | | | |
|---|---|---|---|---|---|
| 1051 | TGCCGGGCAA | GGTCGGCGGG | ATGAGGGCCA | CGAACGCCAG | CACGGCCTGG |
| | ACGGCCCGTT | CCAGCCGCCC | TACTCCCGGT | GCTTGCGGTC | GTGCCGGACC |

------------------------------------------------------

tk gene

. G P L T P P I L A V F A L V A Q P .

| | | | | | |
|---|---|---|---|---|---|
| 1101 | GGGGTCATGC | TGCCCATAAG | GTATCGCGCG | GCCGGGTAGC | ACAGGAGGGC |
| | CCCCAGTACG | ACGGGTATTC | CATAGCGCGC | CGGCCCATCG | TGTCCTCCCG |

------------------------------------------------------

tk gene

.. T M S G M L Y R A A P Y C L L A

| | | | | | |
|---|---|---|---|---|---|
| 1151 | GGCGATGGGA | TGGCGGTCGA | AGATGAGGGT | GAGGGCCGGG | GGCGGGGCAT |
| | CCGCTACCCT | ACCGCCAGCT | TCTACTCCCA | CTCCCGGCCC | CCGCCCCGTA |

------------------------------------------------------

tk gene

A I P H R D F I L T L A P P P A H .

| | | | | | |
|---|---|---|---|---|---|
| 1201 | GTGAGCTCCC | AGCCTCCCCC | CCGATATGAG | GAGCCAGAAC | GGCGTCGGTC |
| | CACTCGAGGG | TCGGAGGGGG | GGCTATACTC | CTCGGTCTTG | CCGCAGCCAG |

------------------------------------------------------

tk gene

. S S G A E G G I H P A L V A D T V .

| | | | | | |
|---|---|---|---|---|---|
| 1251 | ACGGCATAAG | GCATGCCCAT | TGTTATCTGG | GCGCTTGTCA | TTACCACCGC |
| | TGCCGTATTC | CGTACGGGTA | ACAATAGACC | CGCGAACAGT | AATGGTGGCG |

------------------------------------------------------

tk gene

.. A Y P M G M T I Q A S T M V V A

| | | | | | |
|---|---|---|---|---|---|
| 1301 | CGCGTCCCCG | GCCGATATCT | CACCCTGGTC | GAGGCGGTGT | TGTGTGGTGT |
| | GCGCAGGGGC | CGGCTATAGA | GTGGGACCAG | CTCCGCCACA | ACACACCACA |

------------------------------------------------------

tk gene

A D G A S I E G Q D L R H Q T T Y .

| | | | | | |
|---|---|---|---|---|---|
| 1351 | AGATGTTCGC | GATTGTCTCG | GAAGCCCCCA | ACACCCGCCA | GTAAGTCATC |
| | TCTACAAGCG | CTAACAGAGC | CTTCGGGGGT | TGTGGGCGGT | CATTCAGTAG |

------------------------------------------------------

tk gene

. I N A I T E S A G L V R W Y T M P .

| | | | | | |
|---|---|---|---|---|---|
| 1401 | GGCTCGGGTA | CGTAGACGAT | ATCGTCGCGC | GAACCCAGGG | CCACCAGCAG |
| | CCGAGCCCAT | GCATCTGCTA | TAGCAGCGCG | CTTGGGTCCC | GGTGGTCGTC |

------------------------------------------------------

tk gene

.. E P V Y V I D D R S G L A V L L

| | | | | | |
|---|---|---|---|---|---|
| 1451 | TTGCGTGGTG | GTGGTTTTCC | CCATCCCGTG | GGGACCGTCT | ATATAAACCC |
| | AACGCACCAC | CACCAAAAGG | GGTAGGGCAC | CCCTGGCAGA | TATATTTGGG |

------------------------------------------------------

tk gene

Q T T T T K G M G H P G D I Y V R .

| | | | | | |
|---|---|---|---|---|---|
| 1501 | GCAGTAGCGT | GGGCATTTTC | TGCTCCAGGC | GGACTTCCGT | GGCTTTTTGT |
| | CGTCATCGCA | CCCGTAAAAG | ACGAGGTCCG | CCTGAAGGCA | CCGAAAAACA |

------------------------------------------------------

tk gene

. L L T P M K Q E L R V E T A K Q Q .

| | | | | | |
|---|---|---|---|---|---|
| 1551 | TGCCGGCGAG | GGCGCAACGC | CGTACGTCGG | TTGTTATGGC | CGCGAGAACG |
| | ACGGCCGCTC | CCGCGTTGCG | GCATGCAGCC | AACAATACCG | GCGCTCTTGC |

------------------------------------------------------

tk gene

.. R R P R L A T R R N N H G R S R

| | | | | | |
|---|---|---|---|---|---|
| 1601 | CGCAGCCTGG | TCGAACGCAG | ACGCGTGTTG | ATGGCAGGGG | TACGAAGCCA |
| | GCGTCGGACC | AGCTTGCGTC | TGCGCACAAC | TACCGTCCCC | ATGCTTCGGT |

------------------------------------------------------

tk gene

A A Q D F A S A H Q H C P Y S A M .

| | | | | | |
|---|---|---|---|---|---|
| 1651 | TAGATCCCGT | TATCAATTAC | TTATACTATC | CGGCGCGCAA | GCGAGCGTGT |
| | ATCTAGGGCA | ATAGTTAATG | AATATGATAG | GCCGCGCGTT | CGCTCGCACA |

--------------------

ie-0 promoter

| | | | | | |
|---|---|---|---|---|---|
| 1701 | GCGCCGGAGC | ACAATTGATA | CTGATTTACG | AGTTGGGCAA | ACGGGCTTTA |
| | CGCGGCCTCG | TGTTAACTAT | GACTAAATGC | TCAACCCGTT | TGCCCGAAAT |

------------------------------------------------------

ie-0 promoter

| | | | | | |
|---|---|---|---|---|---|
| 1751 | TATAGCCTGT | CCCCTCCACA | GCCCTAGTGC | CGTGCGCAAA | GTGCCTACGT |
| | ATATCGGACA | GGGGAGGTGT | CGGGATCACG | GCACGCGTTT | CACGGATGCA |

------------------------------------------------------

ie-0 promoter

| | | | | | |
|---|---|---|---|---|---|
| 1801 | GACCAGGCTC | TCCTACGCAT | ATACAATCTT | ATCTCTATAG | ATAAGGTTTC |
| | CTGGTCCGAG | AGGATGCGTA | TATGTTAGAA | TAGAGATATC | TATTCCAAAG |

------------------------------------------------------

ie-0 promoter

| | | | | | |
|---|---|---|---|---|---|
| 1851 | CATATATAAA | GCCTCTCGAT | GGCTGAACGT | GCACAGTATC | GTGTTGATTT |
| | GTATATATTT | CGGAGAGCTA | CCGACTTGCA | CGTGTCATAG | CACAACTAAA |

------------------------------------------------------

ie-0 promoter

| | | | | | |
|---|---|---|---|---|---|
| 1901 | CTGAGTGCTA | ACTAACAGTT | ACAATGAACC | GTTTTTTTCG | AGAGAATAAC |
| | GACTCACGAT | TGATTGTCAA | TGTTACTTGG | CAAAAAAAGC | TCTCTTATTG |

------------------------------------------------------

ie-0 promoter

| | | | | | |
|---|---|---|---|---|---|
| 1951 | ATTTTTGACG | CGCCAAGGAC | CGGGGGCAAG | GGTCGTGCCA | AATCTTTGCC |
| | TAAAAACTGC | GCGGTTCCTG | GCCCCCGTTC | CCAGCACGGT | TTAGAAACGG |

------------------------------------------------------

ie-0 promoter

| | | | | | |
|---|---|---|---|---|---|
| 2001 | AGCGCCTGCC | GCCAACTCGC | CGCCGTCGCC | TGTTCGTCCG | CCGCCAAAAT |
| | TCGCGGACGG | CGGTTGAGCG | GCGGCAGCGG | ACAAGCAGGC | GGCGGTTTTA |

------------------------------------------------------

ie-0 promoter

| | | | | | |
|---|---|---|---|---|---|
| 2051 | CTAACATCAA | ACCACCTACG | CGCATCTCTC | CGCCTAAACA | GCCTATGTGC |
| | GATTGTAGTT | TGGTGGATGC | GCGTAGAGAG | GCGGATTTGT | CGGATACACG |

------------------------------------------------------

ie-0 promoter

| | | | | | |
|---|---|---|---|---|---|
| 2101 | ACCTCTCCGG | CCAAGCCGTT | GGAGCACAGC | AGCATTGTAA | GTAAAAAACC |
| | TGGAGAGGCC | GGTTCGGCAA | CCTCGTGTCG | TCGTAACATT | CATTTTTTGG |

------------------------------------------------------

ie-0 promoter

| | | | | | |
|---|---|---|---|---|---|
| 2151 | AGTCGTCAAC | AGAAAAGATG | GATATTTTGT | GCCGCCCGAG | TTTGGGAACA |
| | TCAGCAGTTG | TCTTTTCTAC | CTATAAAACA | CGGCGGGCTC | AAACCCTTGT |

-----------------------------------------------------

ie-0 promoter

| | | | | | |
|---|---|---|---|---|---|
| 2201 | AGTTTGAAGG | TTTGCCCGCG | TACAGCGACA | AACTGGATTT | CAAACAAGAG |
| | TCAAACTTCC | AAACGGGCGC | ATGTCGCTGT | TTGACCTAAA | GTTTGTTCTC |

----------------------------------
ie-0 promoter

p10 promoter

------------------------

| | | | | | |
|---|---|---|---|---|---|
| 2251 | CGCGATCTAC | GTACCTGCAG | GCCCGGGCTC | AACCCAACAC | AATATATTAT |
| | GCGCTAGATG | CATGGACGTC | CGGGCCCGAG | TTGGGTTGTG | TTATATAATA |

p10 promoter

------------------------------------------------------

| | | | | | |
|---|---|---|---|---|---|
| 2301 | AGTTAAATAA | GAATTATTAT | CAAATCATTT | GTATATTAAT | TAAAATACTA |
| | TCAATTTATT | CTTAATAATA | GTTTAGTAAA | CATATAATTA | ATTTTATGAT |

| | |
|---|---|
| p10 promoter | lacZ |

| | |
|---|---|
| ---------------------------- | -------------- |

M T M I T .

| | | | | | |
|---|---|---|---|---|---|
| 2351 | TACTGTAAAT | TACATTTTAT | TTACAATTCA | CTCTAGAATG | ACCATGATTA |
| | ATGACATTTA | ATGTAAAATA | AATGTTAAGT | GAGATCTTAC | TGGTACTAAT |

lacZ

------------------------------------------------------
. D S L A V V L Q R R D W E N P G

| | | | | | |
|---|---|---|---|---|---|
| 2401 | CGGATTCACT | GGCCGTCGTT | TTACAACGTC | GTGACTGGGA | AAACCCTGGC |
| | GCCTAAGTGA | CCGGCAGCAA | AATGTTGCAG | CACTGACCCT | TTTGGGACCG |

lacZ

------------------------------------------------------
V T Q L N R L A A H P P F A S W R .

| | | | | | |
|---|---|---|---|---|---|
| 2451 | GTTACCCAAC | TTAATCGCCT | TGCAGCACAT | CCCCCTTTCG | CCAGCTGGCG |
| | CAATGGGTTG | AATTAGCGGA | ACGTCGTGTA | GGGGGAAAGC | GGTCGACCGC |

lacZ

------------------------------------------------------
. N S E E A R T D R P S Q Q L R S L .

| | | | | | |
|---|---|---|---|---|---|
| 2501 | TAATAGCGAA | GAGGCCCGCA | CCGATCGCCC | TTCCCAACAG | TTGCGCAGCC |
| | ATTATCGCTT | CTCCGGGCGT | GGCTAGCGGG | AAGGGTTGTC | AACGCGTCGG |

lacZ

------------------------------------------------------
. N G E W R F A W F P A P E A V P

| | | | | | |
|---|---|---|---|---|---|
| 2551 | TGAATGGCGA | ATGGCGCTTT | GCCTGGTTTC | CGGCACCAGA | AGCGGTGCCG |
| | ACTTACCGCT | TACCGCGAAA | CGGACCAAAG | GCCGTGGTCT | TCGCCACGGC |

lacZ

------------------------------------------------------

```
        Bsu36I
        --------
```

E S W L E C D L P E A D T V V V P .

| | | | | | |
|---|---|---|---|---|---|
| 2601 | GAAAGCTGGC | TGGAGTGCGA | TCTTCCTGAG | GCCGATACTG | TCGTCGTCCC |
| | CTTTCGACCG | ACCTCACGCT | AGAAGGACTC | CGGCTATGAC | AGCAGCAGGG |

lacZ

------------------------------------------------------
. S N W Q M H G Y D A P I Y T N V T .

| | | | | | |
|---|---|---|---|---|---|
| 2651 | CTCAAACTGG | CAGATGCACG | GTTACGATGC | GCCCATCTAC | ACCAACGTAA |
| | GAGTTTGACC | GTCTACGTGC | CAATGCTACG | CGGGTAGATG | TGGTTGCATT |

lacZ

------------------------------------------------------
. Y P I T V N P P F V P T E N P T

| | | | | | |
|---|---|---|---|---|---|
| 2701 | CCTATCCCAT | TACGGTCAAT | CCGCCGTTTG | TTCCCACGGA | GAATCCGACG |
| | GGATAGGGTA | ATGCCAGTTA | GGCGGCAAAC | AAGGGTGCCT | CTTAGGCTGC |

lacZ

------------------------------------------------------
G C Y S L T F N V D E S W L Q E G .

| | | | | | |
|---|---|---|---|---|---|
| 2751 | GGTTGTTACT | CGCTCACATT | TAATGTTGAT | GAAAGCTGGC | TACAGGAAGG |
| | CCAACAATGA | GCGAGTGTAA | ATTACAACTA | CTTTCGACCG | ATGTCCTTCC |

lacZ

------------------------------------------------------
. Q T R I I F D G V N S A F H L W C .

| | | | | | |
|---|---|---|---|---|---|
| 2801 | CCAGACGCGA | ATTATTTTTG | ATGGCGTTAA | CTCGGCGTTT | CATCTGTGGT |
| | GGTCTGCGCT | TAATAAAAAC | TACCGCAATT | GAGCCGCAAA | GTAGACACCA |

lacZ

------------------------------------------------------
. N G R W V G Y G Q D S R L P S E

| | | | | | |
|---|---|---|---|---|---|
| 2851 | GCAACGGGCG | CTGGGTCGGT | TACGGCCAGG | ACAGTCGTTT | GCCGTCTGAA |
| | CGTTGCCCGC | GACCCAGCCA | ATGCCGGTCC | TGTCAGCAAA | CGGCAGACTT |

lacZ

------------------------------------------------------
F D L S A F L R A G E N R L A V M .

| | | | | | |
|---|---|---|---|---|---|
| 2901 | TTTGACCTGA | GCGCATTTTT | ACGCGCCGGA | GAAAACCGCC | TCGCGGTGAT |
| | AAACTGGACT | CGCGTAAAAA | TGCGCGGCCT | CTTTTGGCGG | AGCGCCACTA |

lacZ

------------------------------------------------------
. V L R W S D G S Y L E D Q D M W R .

| | | | | | |
|---|---|---|---|---|---|
| 2951 | GGTGCTGCGT | TGGAGTGACG | GCAGTTATCT | GGAAGATCAG | GATATGTGGC |
| | CCACGACGCA | ACCTCACTGC | CGTCAATAGA | CCTTCTAGTC | CTATACACCG |

lacZ

------------------------------------------------------
. M S G I F R D V S L L H K P T T

| | | | | | |
|---|---|---|---|---|---|
| 3001 | GGATGAGCGG | CATTTTCCGT | GACGTCTCGT | TGCTGCATAA | ACCGACTACA |
| | CCTACTCGCC | GTAAAAGGCA | CTGCAGAGCA | ACGACGTATT | TGGCTGATGT |

lacZ

------------------------------------------------------
Q I S D F H V A T R F N D D F S R .

| | | | | | |
|---|---|---|---|---|---|
| 3051 | CAAATCAGCG | ATTTCCATGT | TGCCACTCGC | TTTAATGATG | ATTTCAGCCG |
| | GTTTAGTCGC | TAAAGGTACA | ACGGTGAGCG | AAATTACTAC | TAAAGTCGGC |

lacZ

------------------------------------------------------
. A V L E A E V Q M C G E L R D Y L .

| | | | | | |
|---|---|---|---|---|---|
| 3101 | CGCTGTACTG | GAGGCTGAAG | TTCAGATGTG | CGGCGAGTTG | CGTGACTACC |
| | GCGACATGAC | CTCCGACTTC | AAGTCTACAC | GCCGCTCAAC | GCACTGATGG |

lacZ

------------------------------------------------------
. R V T V S L W Q G E T Q V A S G

| | | | | | |
|---|---|---|---|---|---|
| 3151 | TACGGGTAAC | AGTTTCTTTA | TGGCAGGGTG | AAACGCAGGT | CGCCAGCGGC |
| | ATGCCCATTG | TCAAAGAAAT | ACCGTCCCAC | TTTGCGTCCA | GCGGTCGCCG |

lacZ

------------------------------------------------------
T A P F G G E I I D E R G G Y A D .

| | | | | | |
|---|---|---|---|---|---|
| 3201 | ACCGCGCCTT | TCGGCGGTGA | AATTATCGAT | GAGCGTGGTG | GTTATGCCGA |
| | TGGCGCGGAA | AGCCGCCACT | TTAATAGCTA | CTCGCACCAC | CAATACGGCT |

lacZ

------------------------------------------------------
. R V T L R L N V E N P K L W S A E .

| | | | | | |
|---|---|---|---|---|---|
| 3251 | TCGCGTCACA | CTACGTCTGA | ACGTCGAAAA | CCCGAAACTG | TGGAGCGCCG |
| | AGCGCAGTGT | GATGCAGACT | TGCAGCTTTT | GGGCTTTGAC | ACCTCGCGGC |

lacZ

------------------------------------------------------
. I P N L Y R A V V E L H T A D G

| | | | | | |
|---|---|---|---|---|---|
| 3301 | AAATCCCGAA | TCTCTATCGT | GCGGTGGTTG | AACTGCACAC | CGCCGACGGC |
| | TTTAGGGCTT | AGAGATAGCA | CGCCACCAAC | TTGACGTGTG | GCGGCTGCCG |

lacZ

------------------------------------------------------
T L I E A E A C D V G F R E V R I .

| | | | | | |
|---|---|---|---|---|---|
| 3351 | ACGCTGATTG | AAGCAGAAGC | CTGCGATGTC | GGTTTCCGCG | AGGTGCGGAT |
| | TGCGACTAAC | TTCGTCTTCG | GACGCTACAG | CCAAAGGCGC | TCCACGCCTA |

lacZ

------------------------------------------------------
. E N G L L L L N G K P L L I R G V .

| | | | | | |
|---|---|---|---|---|---|
| 3401 | TGAAAATGGT | CTGCTGCTGC | TGAACGGCAA | GCCGTTGCTG | ATTCGAGGCG |
| | ACTTTTACCA | GACGACGACG | ACTTGCCGTT | CGGCAACGAC | TAAGCTCCGC |

lacZ

------------------------------------------------------
. N R H E H H P L H G Q V M D L Q

| | | | | | |
|---|---|---|---|---|---|
| 3451 | TTAACCGTCA | CGAGCATCAT | CCTCTGCATG | GTCAGGTCAT | GGATGAGCAG |
| | AATTGGCAGT | GCTCGTAGTA | GGAGACGTAC | CAGTCCAGTA | CCTACTCGTC |

lacZ

------------------------------------------------------
T M V Q D I L L M K Q N N F N A V .

| | | | | | |
|---|---|---|---|---|---|
| 3501 | ACGATGGTGC | AGGATATCCT | GCTGATGAAG | CAGAACAACT | TTAACGCCGT |
| | TGCTACCACG | TCCTATAGGA | CGACTACTTC | GTCTTGTTGA | AATTGCGGCA |

lacZ

------------------------------------------------------
. R C S H Y P N H P L W Y T L C D R .

| | | | | | |
|---|---|---|---|---|---|
| 3551 | GCGCTGTTCG | CATTATCCGA | ACCATCCGCT | GTGGTACACG | CTGTGCGACC |
| | CGCGACAAGC | GTAATAGGCT | TGGTAGGCGA | CACCATGTGC | GACACGCTGG |

lacZ

------------------------------------------------------
. Y G L Y V V D E A N I E T H G M

| | | | | | |
|---|---|---|---|---|---|
| 3601 | GCTACGGCCT | GTATGTGGTG | GATGAAGCCA | ATATTGAAAC | CCACGGCATG |
| | CGATGCCCGA | CATACACCAC | CTACTTCGGT | TATAACTTTG | GGTGCCGTAC |

lacZ

------------------------------------------------------
V P M N R L T D D P R W L P A M S .

| | | | | | |
|---|---|---|---|---|---|
| 3651 | GTGCCAATGA | ATCGTCTGAC | CGATGATCCG | CGCTGGCTAC | CGGCGATGAG |
| | CACGGTTACT | TAGCAGACTG | GCTACTAGGC | GCGACCGATG | GCCGCTACTC |

lacZ

------------------------------------------------------
. E R V T R M V Q R D R N H P S V I .

| | | | | | |
|---|---|---|---|---|---|
| 3701 | CGAACGCGTA | ACGCGAATGG | TGCAGCGCGA | TCGTAATCAC | CCGAGTGTGA |
| | GCTTGCGCAT | TGCGCTTACC | ACGTCGCGCT | AGCATTAGTG | GGCTCACACT |

lacZ

------------------------------------------------------
. I W S L G N E S G H G A N H D A

| | | | | | |
|---|---|---|---|---|---|
| 3751 | TCATCTGGTC | GCTGGGGAAT | GAATCAGGCC | ACGGCGCTAA | TCACGACGCG |
| | AGTAGACCAG | CGACCCCTTA | CTTAGTCCGG | TGCCGCGATT | AGTGCTGCGC |

lacZ

------------------------------------------------------
L Y R W I K S V D P S R P V Q Y E .

| | | | | | |
|---|---|---|---|---|---|
| 3801 | CTGTATCGCT | GGATCAAATC | TGTCGATCCT | TCCCGCCCGG | TGCAGTATGA |
| | GACATAGCGA | CCTAGTTTAG | ACAGCTAGGA | AGGGCGGGCC | ACGTCATACT |

lacZ

------------------------------------------------------
. G G G A D T T A T D I I C P M Y A .

| | | | | | |
|---|---|---|---|---|---|
| 3851 | AGGCGGCGGA | GCCGACACCA | CGGCCACCGA | TATTATTTGC | CCGATGTACG |
| | TCCGCCGCCT | CGGCTGTGGT | GCCGGTGGCT | ATAATAAACG | GGCTACATGC |

lacZ

------------------------------------------------------
. R V D E D Q P F P A V P K W S I

| | | | | | |
|---|---|---|---|---|---|
| 3901 | CGCGCGTGGA | TGAAGACCAG | CCCTTCCCGG | CTGTGCCGAA | ATGGTCCATC |
| | GCGCGCACCT | ACTTCTGGTC | GGGAAGGGCC | GACACGGCTT | TACCAGGTAG |

lacZ

------------------------------------------------------
K K W L S L P G E T R P L I L C E .

| | | | | | |
|---|---|---|---|---|---|
| 3951 | AAAAAATGGC | TTTCGCTACC | TGCAGAGACG | CCCCCGCTGA | TCCTTTGCCA |
| | TTTTTTACCG | AAAGCGATGG | ACCTCTCTGC | GCGGGCCACT | AGGAAACGCT |

lacZ

------------------------------------------------------
. Y A H A M G N S L G G F A K Y W Q .

| | | | | | |
|---|---|---|---|---|---|
| 4001 | ATACGCCCAC | GCGATGCGTA | ACAGTCTTGG | CGGTTTCGCT | AAATACTGGC |
| | TATGCGGGTG | CGCTACCCAT | TGTCAGAACC | GCCAAAGCGA | TTTATGACCG |

lacZ

------------------------------------------------------
. A F R Q Y P R L Q G G F V W D W

| | | | | | |
|---|---|---|---|---|---|
| 4051 | AGGCGTTTCG | TCAGTATCCC | CGTTTACAGG | GCGGCTTCGT | CTGGGACTCG |
| | TCCGCAAAGC | AGTCATACGG | GCAAATGTCC | CGCCGAACCA | CACCCTGACC |

lacZ

------------------------------------------------------
V D Q S L I K Y D E N G N P W S A .

| | | | | | |
|---|---|---|---|---|---|
| 4101 | GTGCATCAGT | CGCTGATTAA | ATATGATCAA | AACGCCAACC | CGTGGTCGGC |
| | CACCTAGTCA | GCCACTAATT | TATACTACTT | TTGCCCTTGC | GCACCAGCCC |

lacZ

------------------------------------------------------
. Y G G D F G D T P N D R Q F C M N .

| | | | | | |
|---|---|---|---|---|---|
| 4151 | TTACCCCCCT | CATTTTCCCG | ATACCCCCAA | CCATCCCCAC | TTCTCTATCA |
| | AATCCCCCCA | CTAAAACCCC | TATCCCCCTT | CCTAGCCCTC | AACACATACT |

lacZ

------------------------------------------------------
. G L V F A D R T P H P A L T E A

| | | | | | |
|---|---|---|---|---|---|
| 4201 | ACCCTCTCCT | CTTTGCCCAC | CCCACCCCCC | ATCCACCCCT | CACCCAAGCA |
| | TGCCAGACCA | CAAACCCCTG | CCCTCCCCCC | TACCTCGCCA | CTCCCTTCCT |

lacZ

------------------------------------------------------
K H Q Q Q F F Q F R L S G Q T I E .

| | | | | | |
|---|---|---|---|---|---|
| 4251 | AAACACCACC | ACCAGTTTTT | CCACTTCCCT | TTATCCGCCC | AAACCATCCA |
| | TTTGTCCTCG | TCGTCAAAAA | CCTCAACCCA | AATACCCCCC | TTTCCTACCT |

lacZ

------------------------------------------------------
. V T S E Y L F R H S D N E L L H W .

| | | | | | |
|---|---|---|---|---|---|
| 4301 | ACTCACCACC | CAATACCTCT | TCCCTCATAC | CGATAACCAC | CTCCTCCACT |
| | TCACTCCTCG | CTTATCCACA | AGCCACTATC | CCTATTGCTC | CACCACCTCA |

lacZ

------------------------------------------------------
. M V A L D G K P L A S G E V P L

| | | | | | |
|---|---|---|---|---|---|
| 4351 | GGATCCTCCC | CCTCCATCCT | AACCCCCTCC | CAAGCCCTCA | ACTCCCTCTG |
| | CCTACCACCC | CCACCTACCA | TTCCCCCACC | CTTCCCCACT | TCACCCACAC |

lacZ

------------------------------------------------------
D V A P Q G K Q L I E L P E L P Q .

| | | | | | |
|---|---|---|---|---|---|
| 4401 | GATGTCGCTC | CACAAGGTAA | ACAGTTGATT | GAACTGCCTG | AACTACCGCA |
| | CTACAGCGAG | GTGTTCCATT | TGTCAACTAA | CTTGACGGAC | TTGATGGCGT |

lacZ

------------------------------------------------------
. P E S A G Q L W L T V R V V Q P N .

| | | | | | |
|---|---|---|---|---|---|
| 4451 | GCCGGAGAGC | GCCGGGCAAC | TCTGGCTCAC | AGTACGCGTA | GTGCAACCGA |
| | CGGCCTCTCG | CGGCCCGTTG | AGACCGAGTG | TCATGCGCAT | CACGTTGGCT |

lacZ

------------------------------------------------------
. A T A W S E A G H I S A W Q Q W

| | | | | | |
|---|---|---|---|---|---|
| 4501 | ACGCGACCGC | ATGGTCAGAA | GCCGGGCACA | TCAGCGCCTG | GCAGCAGTGG |
| | TGCGCTGGCG | TACCAGTCTT | CGGCCCGTGT | AGTCGCGGAC | CGTCGTCACC |

lacZ

------------------------------------------------------
R L A E N L S V T L P A A S H A I .

| | | | | | |
|---|---|---|---|---|---|
| 4551 | CGTCTGGCGG | AAAACCTCAG | TGTGACGCTC | CCCGCCGCGT | CCCACGCCAT |
| | GCAGACCGCC | TTTTGGAGTC | ACACTGCGAG | GGGCGGCGCA | GGGTGCGGTA |

lacZ

------------------------------------------------------
. P H L T T S E M D F C I E L G N K .

| | | | | | |
|---|---|---|---|---|---|
| 4601 | CCCGCATCTG | ACCACCAGCG | AAATGGATTT | TTGCATCGAG | CTGGGTAATA |
| | GGGCGTAGAC | TGGTGGTCGC | TTTACCTAAA | AACGTAGCTC | GACCCATTAT |

lacZ

------------------------------------------------------
. R W Q F N R Q S G F L S Q M W I

| | | | | | |
|---|---|---|---|---|---|
| 4651 | AGCGTTGGCA | ATTTAACCGC | CAGTCAGGCT | TTCTTTCACA | GATGTGGATT |
| | TCGCAACCGT | TAAATTGGCG | GTCAGTCCGA | AAGAAAGTGT | CTACACCTAA |

lacZ

------------------------------------------------------
G D K K Q L L T P L R D Q F T R A .

| | | | | | |
|---|---|---|---|---|---|
| 4701 | GGCGATAAAA | AACAACTGCT | GACGCCGCTG | CGCGATCAGT | TCACCCGTGC |
| | CCGCTATTTT | TTGTTGACGA | CTGCGGCGAC | GCGCTAGTCA | AGTGGGCACG |

lacZ

------------------------------------------------------
. P L D N D I G V S E A T R I D P N .

| | | | | | |
|---|---|---|---|---|---|
| 4751 | ACCGCTGGAT | AACGACATTG | GCGTAAGTGA | AGCGACCCGC | ATTGACCCTA |
| | TGGCGACCTA | TTGCTGTAAC | CGCATTCACT | TCGCTGGGCG | TAACTGGGAT |

lacZ

------------------------------------------------------
. A W V E R W K A A G H Y Q A E A

| | | | | | |
|---|---|---|---|---|---|
| 4801 | ACGCCTGGGT | CGAACGCTGG | AAGGCGGCGG | GCCATTACCA | GGCCGAAGCA |
| | TGCGGACCCA | GCTTGCGACC | TTCCGCCGCC | CGGTAATGGT | CCGGCTTCGT |

lacZ

------------------------------------------------------
A L L Q C T A D T L A D A V L I T .

| | | | | | |
|---|---|---|---|---|---|
| 4851 | GCGTTGTTGC | AGTGCACGGC | AGATACACTT | GCTGATGCGG | TGCTGATTAC |
| | CGCAACAACG | TCACGTGCCG | TCTATGTGAA | CGACTACGCC | ACGACTAATG |

lacZ

------------------------------------------------------
. T A H A W Q H Q G K T L F I S R K .

| | | | | | |
|---|---|---|---|---|---|
| 4901 | GACCGCTCAC | GCGTGGCAGC | ATCAGGGGAA | AACCTTATTT | ATCAGCCGGA |
| | CTGGCGAGTG | CGCACCGTCG | TAGTCCCCTT | TTGGAATAAA | TAGTCGGCCT |

lacZ

------------------------------------------------------
. T Y R I D G S G Q M A I T V D V

| | | | | | |
|---|---|---|---|---|---|
| 4951 | AAACCTACCG | GATTGATGGT | AGTGGTCAAA | TGGCGATTAC | CGTTGATGTT |
| | TTTGGATGGC | CTAACTACCA | TCACCAGTTT | ACCGCTAATG | GCAACTACAA |

lacZ

------------------------------------------------------
E V A S D T P H P A R I G L N C Q .

| | | | | | |
|---|---|---|---|---|---|
| 5001 | GAAGTGGCGA | GCGATACACC | GCATCCGGCG | CGGATTGGCC | TGAACTGCCA |
| | CTTCACCGCT | CGCTATGTGG | CGTAGGCCGC | GCCTAACCGG | ACTTGACGGT |

lacZ

------------------------------------------------------
. L A Q V A E R V N W L G L G P Q E .

| | | | | | |
|---|---|---|---|---|---|
| 5051 | GCTGGCGCAG | GTACCAGAGC | GGGTAAACTC | GCTCGGATTA | GGGCCGCAAG |
| | CGACCGCGTC | CATCGTCTCG | CCCATTTGAC | CGACCCTAAT | CCCCGCGTTC |

lacZ

------------------------------------------------------
. N Y P D R L T A A C F D R W D L

| | | | | | |
|---|---|---|---|---|---|
| 5101 | AAAACTATCC | CGACCGCCTT | ACTCCCGCCT | GTTTTCACCG | CTGGGATCTG |
| | TTTTGATAGG | CCTGGCGGAA | TGACGGCCGA | CAAAACTGGC | GACCCTAGAC |

lacZ

------------------------------------------------------
P L S D M Y T P Y V F P S E N G L .

| | | | | | |
|---|---|---|---|---|---|
| 5151 | CCATTCTCAG | ACATGTATAC | CCCGTACGTC | TTCCCGAGCC | AAAACGGTCT |
| | GGTAACAGTC | TCTACATATC | GCGCATGCAC | AAGGCCTCCC | TTTTGCCAGA |

lacZ

------------------------------------------------------
. R C G T R E L N Y G P H Q W R G D .

| | | | | | |
|---|---|---|---|---|---|
| 5201 | GCGCTGCCGG | ACGCGCGAAT | TGAATTATGG | CCCACACCAG | TGCCGCCGCG |
| | CGCCACGCCC | TGCCCGCTTA | ACTTAATACC | CGCTGTGGTC | ACCGCGCCGC |

lacZ

------------------------------------------------------
. F Q F N I S R Y S Q Q Q L M E T

| | | | | | |
|---|---|---|---|---|---|
| 5251 | ACTTCCAGTT | CAACATCAGC | CGCTACACTC | AACAGCAACT | GATCGAAACC |
| | TCAAGCTCAA | GTTGTAGTCG | CCGATGTCAG | TTGTCGTTCA | CTACCTTTGG |

lacZ

------------------------------------------------------
S H R H L L H A E E G T W L N I D .

| | | | | | |
|---|---|---|---|---|---|
| 5301 | AGCCATCGCC | ATCTGCTGCA | CGCGGAAGAA | GGCACATGGC | TGAATATCGA |
| | TCGGTAGCGG | TAGACGACGT | GCGCCTTCTT | CCGTGTACCG | ACTTATAGCT |

lacZ

------------------------------------------------------
. G F H M G I G G D D S W S P S V S .

| | | | | | |
|---|---|---|---|---|---|
| 5351 | CGGTTTCCAT | ATGGGGATTG | GTGGCGACGA | CTCCTGGAGC | CCGTCAGTAT |
| | GCCAAAGGTA | TACCCCTAAC | CACCGCTGCT | GAGGACCTCG | GGCAGTCATA |

lacZ

------------------------------------------------------
. A E F Q L S A G R Y H Y Q L V W

| | | | | | |
|---|---|---|---|---|---|
| 5401 | CGGCGGAATT | CCAGCTGAGC | GCCGGTCGCT | ACCATTACCA | GTTGGTCTGG |
| | GCCGCCTTAA | GGTCGACTCG | CGGCCAGCGA | TGGTAATGGT | CAACCAGACC |

| | |
|---|---|
| lacZ | AttR2 |

| | |
|---|---|
| --------- | ------------------------- |
| C Q K | |

| | | | | | |
|---|---|---|---|---|---|
| 5451 | TGTCAAAAAT | AATGACTGCA | GGTCGACCAT | AGTGACTGGA | TATGTTGTGT |
| | ACAGTTTTTA | TTACTGACGT | CCAGCTGGTA | TCACTGACCT | ATACAACACA |

AttR2

------------------------------------------------------

| | | | | | |
|---|---|---|---|---|---|
| 5501 | TTTACAGTAT | TATGTAGTCT | GTTTTTTATG | CAAAATCTAA | TTTAATATAT |
| | AAATGTCATA | ATACATCAGA | CAAAAAATAC | GTTTTAGATT | AAATTATATA |

AttR2

------------------------------------------------------

| | | | | | |
|---|---|---|---|---|---|
| 5551 | TGATATTTAT | ATCATTTTAC | GTTTCTCGTT | CAGCTTTCTT | GTACAAAGTG |
| | ACTATAAATA | TAGTAAAATG | CAAAGAGCAA | GTCGAAAGAA | CATGTTTCAC |

| | |
|---|---|
| AttR2 | V5/His |

| | |
|---|---|
| -- | ------------------------------- |

G K P I P N P L L G .

| | | | | | |
|---|---|---|---|---|---|
| 5601 | GTGAGAATGA | ATGAAGATCT | GGGGAAGCCT | ATCCCTAACC | CTCTCCTCGG |
| | CACTCTTACT | TACTTCTAGA | CCCCTTCGGA | TAGGGATTGG | GAGAGGAGCC |

V5/His

--------------------------------------------
. L D S T R T G H H H H H H

| | | | | | |
|---|---|---|---|---|---|
| 5651 | TCTCGATTCT | ACGCGTACCG | GTCATCATCA | CCATCACCAT | TGA |
| | AGAGCTAAGA | TGCGCATGGC | CAGTAGTAGT | GGTAGTGGTA | ACT |

TABLE 14
Nucleotide sequence of the Mel/V5-His DEST cassette.

ph promoter
-----

| | | | | | |
|---|---|---|---|---|---|
| 1 | ATAAGTATTT | TACTGTTTTC | GTAACAGTTT | TGTAATAAAA | AAACCTATAA |
| | TATTCATAAA | ATGACAAAAG | CATTGTCAAA | ACATTATTTT | TTTGGATATT |
| 51 | ATATTCCGGA | TTATTCATAC | CGTCCCACCA | TCGGGCGCGG | ATCCTATAAA |
| | TATAAGGCCT | AATAAGTATG | GCAGGGTGGT | AGCCCGCGCC | TAGGATATTT |

Melittin signal

```
  -----------------------------------------------------
    M K F L V N V A L V F M V V Y I S .
```

| | | | | | |
|---|---|---|---|---|---|
| 101 | TATGAAATTC | TTAGTCAACG | TTGCCCTTGT | TTTTATGGTC | GTATACATTT |
| | ATACTTTAAG | AATCAGTTGC | AACGGGAACA | AAAATACCAG | CATATGTAAA |

| | |
|---|---|
| Melittin signal | attR1 |

| | |
|---|---|
| --------------- | ----------------------- |
| . Y I Y A | |

| | | | | | |
|---|---|---|---|---|---|
| 151 | CTTACATCTA | TGCGGCATGG | TCGAATCAAA | CAAGTTTGTA | CAAAAAAGCT |
| | GAATGTAGAT | ACGCCGTACC | AGCTTAGTTT | GTTCAAACAT | GTTTTTTCGA |

attR1

------------------------------------------------------

| | | | | | |
|---|---|---|---|---|---|
| 201 | GAACGAGAAA | CGTAAAATGA | TATAAATATC | AATATATTAA | ATTAGATTTT |
| | CTTGCTCTTT | GCATTTTACT | ATATTTATAG | TTATATAATT | TAATCTAAAA |

attR1

-----------------------------

| | | | | | |
|---|---|---|---|---|---|
| 251 | GCATAAAAAA | CAGACTACAT | AATACTGTAA | AACACAACAT | ATCCAGTCAC |
| | CGTATTTTTT | GTCTGATGTA | TTATGACATT | TTGTGTTGTA | TAGGTCAGTG |
| 301 | TATGGCGGCC | GCTCCCTAAC | CCACGGGGCC | CGTGGCTATG | GCAGGGCTTG |
| | ATACCGCCGG | CGAGGGATTG | GGTGCCCCGG | GCACCGATAC | CGTCCCGAAC |
| 351 | CCGCCCCGAC | GTTGGCTGCG | AGCCCTGGGC | CTTCACCCGA | ACTTGGGGGT |
| | GGCGGGGCTG | CAACCGACGC | TCGGGACCCG | GAAGTGGGCT | TGAACCCCCA |
| 401 | TGGGGTGGGG | AAAAGGAAGA | AACGCGGGCG | TATTGGTCCC | AATGGGGTCT |
| | ACCCCACCCC | TTTTCCTTCT | TTGCGCCCGC | ATAACCAGGG | TTACCCCAGA |
| 451 | CGGTGGGGTA | TCGACAGAGT | GCCAGCCCTG | GGACCGAACC | CCGCGTTTAT |
| | GCCACCCCAT | AGCTGTCTCA | CGGTCGGGAC | CCTGGCTTGG | GGCGCAAATA |
| 501 | GAACAAACGA | CCCAACACCC | GTGCGTTTTA | TTCTGTCTTT | TTATTGCCGT |
| | CTTGTTTGCT | GGGTTGTGGG | CACGCAAAAT | AAGACAGAAA | AATAACGGCA |
| 551 | CATAGCGCGG | GTTCCTTCCG | GTATTGTCTC | CTTCCGTGTT | TCAGTTAGCC |
| | GTATCGCGCC | CAAGGAAGGC | CATAACAGAG | GAAGGCACAA | AGTCAATCGG |

```
      ---
      tk gene
        N A E .
```

| | | | | | |
|---|---|---|---|---|---|
| 601 | TCCCCCATCT | CCCGGGCAAA | CGTGCGCGCC | AGGTCGCAGA | +L,32 TCGTCGGTAT |
| | AGGGGGTAGA | GGGCCCGTTT | GCACGCGCGG | TCCAGCGTCT | AGCAGCCATA |

------------------------------------------------------

tk gene

.. G M E R A F T R A L D C I T P I

| | | | | | |
|---|---|---|---|---|---|
| 651 | GGAGCCTGGG | GTGGTGACGT | GGGTCTGGAC | CATCCCGGAG | GTAAGTTGCA |
| | CCTCGGACCC | CACCACTGCA | CCCAGACCTG | GTAGGGCCTC | CATTCAACGT |

------------------------------------------------------

tk gene

S G P T T V H T Q V M G S T L Q L .

| | | | | | |
|---|---|---|---|---|---|
| 701 | GCAGGGCGTC | CCGGCAGCCG | GCGGGCGATT | GGTCGTAATC | CAGGATAAAG |
| | CGTCCCGCAG | GGCCGTCGGC | CGCCCGCTAA | CCAGCATTAG | GTCCTATTTC |

------------------------------------------------------

tk gene

. L A D R C G A P S Q D Y D L I F V .

| | | | | | |
|---|---|---|---|---|---|
| 751 | ACATGCATGG | GACGGAGGCG | TTTGGCCAAG | ACGTCCAAAG | CCCAGGCAAA |
| | TGTACGTACC | CTGCCTCCGC | AAACCGGTTC | TGCAGGTTTC | GGGTCCGTTT |

------------------------------------------------------

tk gene

.. H M P R L R K A L V D L A W A F

| | | | | | |
|---|---|---|---|---|---|
| 801 | CACGTTATAC | AGGTCGCCGT | TGGGGGCCAG | CAACTCGGGG | GCCCGAAACA |
| | GTGCAATATG | TCCAGCGGCA | ACCCCCGGTC | GTTGAGCCCC | CGGGCTTTGT |

------------------------------------------------------

tk gene

V N Y L D G N P A L L E P A R F L .

| | | | | | |
|---|---|---|---|---|---|
| 851 | GGGTAAATAA | CGTGTCCCCG | ATATGGGGTC | GTGGGCCCGC | GTTGCTCTGG |
| | CCCATTTATT | GCACAGGGGC | TATACCCCAG | CACCCGGGCG | CAACGAGACC |

------------------------------------------------------

tk gene

. T F L T D G I H P R P G A N S Q P .

| | | | | | |
|---|---|---|---|---|---|
| 901 | GGCTCGGCAC | CCTGGGGCGG | CACGGCCGCC | CCCGAAAGCT | GTCCCCAATC |
| | CCGAGCCGTG | GGACCCCGCC | GTGCCGGCGG | GGGCTTTCGA | CAGGGGTTAG |

------------------------------------------------------

tk gene

.. E A G Q P P V A A G S L Q G W D

| | | | | | |
|---|---|---|---|---|---|
| 951 | CTCCCGCCAC | GACCCGCCGC | CCTGCAGATA | CCGCACCGTA | TTGGCAAGCA |
| | GAGGGCGGTG | CTGGGCGGCG | GGACGTCTAT | GGCGTGGCAT | AACCGTTCGT |

------------------------------------------------------

tk gene

E R W S G G G Q L Y R V T N A L L .

| | | | | | |
|---|---|---|---|---|---|
| 1001 | GCCCATAAAC | GCGGCGAATC | GCGGCCAGCA | TAGCCAGGTC | AAGCCGCTCG |
| | CGGGTATTTG | CGCCGCTTAG | CGCCGGTCGT | ATCGGTCCAG | TTCGGCGAGC |

------------------------------------------------------

tk gene

. G Y V R R I A A L M A L D L R E G .

| | | | | | |
|---|---|---|---|---|---|
| 1051 | CCGGGGCGCT | GGCGTTTGGC | CAGGCGGTCG | ATGTGTCTGT | CCTCCGGAAG |
| | GGCCCCGCGA | CCGCAAACCG | GTCCGCCAGC | TACACAGACA | GGAGGCCTTC |

------------------------------------------------------

tk gene

.. P R Q R K A L R D I H R D E P L

| | | | | | |
|---|---|---|---|---|---|
| 1101 | GGCCCCCAAC | ACGATGTTTG | TGCCGGGCAA | GGTCGGCGGG | ATGAGGGCCA |
| | CCGGGGGTTG | TGCTACAAAC | ACGGCCCGTT | CCAGCCGCCC | TACTCCCGGT |

------------------------------------------------------

tk gene

A G L V I N T G P L T P P I L A V .

| | | | | | |
|---|---|---|---|---|---|
| 1151 | CGAACGCCAG | CACGGCCTGG | GGGGTCATGC | TGCCCATAAG | GTATCGCGCG |
| | GCTTGCGGTC | GTGCCGGACC | CCCCAGTACG | ACGGGTATTC | CATAGCGCGC |

------------------------------------------------------

tk gene

. F A L V A Q P T M S G M L Y R A A .

| | | | | | |
|---|---|---|---|---|---|
| 1201 | GCCGGGTAGC | ACAGGAGGGC | GGCGATGGGA | TGGCGGTCGA | AGATGAGGGT |
| | CGGCCCATCG | TGTCCTCCCG | CCGCTACCCT | ACCGCCAGCT | TCTACTCCCA |

------------------------------------------------------

tk gene

.. P Y C L L A A I P H R D F I L T

| | | | | | |
|---|---|---|---|---|---|
| 1251 | GAGGGCCGGG | GGCGGGGCAT | GTGAGCTCCC | AGCCTCCCCC | CCGATATGAG |
| | CTCCCGGCCC | CCGCCCCGTA | CACTCGAGGG | TCGGAGGGGG | GGCTATACTC |

------------------------------------------------------

tk gene

L A P P P A H S S G A E G G I H P .

| | | | | | |
|---|---|---|---|---|---|
| 1301 | GAGCCAGAAC | GGCGTCGGTC | ACGGCATAAG | GCATGCCCAT | TGTTATCTGG |
| | CTCGGTCTTG | CCGCAGCCAG | TGCCGTATTC | CGTACGGGTA | ACAATAGACC |

------------------------------------------------------

tk gene

. A L V A D T V A Y P M G M T I Q A .

| | | | | | |
|---|---|---|---|---|---|
| 1351 | GCGCTTGTCA | TTACCACCGC | CGCGTCCCCG | GCCGATATCT | CACCCTGGTC |
| | CGCGAACAGT | AATGGTGGCG | GCGCAGGGGC | CGGCTATAGA | GTGGGACCAG |

------------------------------------------------------

tk gene

.. S T M V V A A D G A S I E G Q D

| | | | | | |
|---|---|---|---|---|---|
| 1401 | GAGGCGGTGT | TGTGTGGTGT | AGATGTTCGC | GATTGTCTCG | GAAGCCCCCA |
| | CTCCGCCACA | ACACACCACA | TCTACAAGCG | CTAACAGAGC | CTTCGGGGGT |

------------------------------------------------------

tk gene

L R H Q T T Y I N A I T E S A G L .

| | | | | | |
|---|---|---|---|---|---|
| 1451 | ACACCCGCCA | GTAAGTCATC | GGCTCGGGTA | CGTAGACGAT | ATCGTCGCGC |
| | TGTGGGCGGT | CATTCAGTAG | CCGAGCCCAT | GCATCTGCTA | TAGCAGCGCG |

------------------------------------------------------

tk gene

. V R W Y T M P E P V Y V I D D R S .

| | | | | | |
|---|---|---|---|---|---|
| 1501 | GAACCCAGGG | CCACCAGCAG | TTGCGTGGTG | GTGGTTTTCC | CCATCCCGTG |
| | CTTGGGTCCC | GGTGGTCGTC | AACGCACCAC | CACCAAAAGG | GGTAGGGCAC |

------------------------------------------------------

tk gene

.. G L A V L L Q T T T T K G M G H

| | | | | | |
|---|---|---|---|---|---|
| 1551 | GGGACCGTCT | ATATAAACCC | GCAGTAGCGT | GGGCATTTTC | TGCTCCAGGC |
| | CCCTGGCAGA | TATATTTGGG | CGTCATCGCA | CCCGTAAAAG | ACGAGGTCCG |

------------------------------------------------------

tk gene

P G D I Y V R L L T P M K Q E L R .

| | | | | | |
|---|---|---|---|---|---|
| 1601 | GGACTTCCGT | GGCTTTTTGT | TGCCGGCGAG | GGCGCAACGC | CGTACGTCGG |
| | CCTGAAGGCA | CCGAAAAACA | ACGGCCGCTC | CCGCGTTGCG | GCATGCAGCC |

------------------------------------------------------

tk gene

. V E T A K Q Q R R P R L A T R R N .

| | | | | | |
|---|---|---|---|---|---|
| 1651 | TTGTTATGGC | CGCGAGAACG | CGCAGCCTGG | TCGAACGCAG | ACGCGTGTTG |
| | AACAATACCG | GCGCTCTTGC | GCGTCGGACC | AGCTTGCGTC | TGCGCACAAC |

------------------------------------------------------

tk gene

.. N H G R S R A A Q D F A S A H Q

| | | | | | |
|---|---|---|---|---|---|
| 1701 | ATGGCAGGGG | TACGAAGCCA | TAGATCCCGT | TATCAATTAC | TTATACTATC |
| | TACCGTCCCC | ATGCTTCGGT | ATCTAGGGCA | ATAGTTAATG | AATATGATAG |

-----------------------

| | |
|---|---|
| tk gene | ie-0 |

```
    pr
  H C P Y S A M
```

| | | | | | |
|---|---|---|---|---|---|
| 1751 | CGGCGCGCAA | GCGAGCGTGT | GCGCCGGAGC | ACAATTGATA | CTGATTTACG |
| | GCCGCGCGTT | CGCTCGCACA | CGCGGCCTCG | TGTTAACTAT | GACTAAATGC |

------------------------------------------------------

ie-0 pr

| | | | | | |
|---|---|---|---|---|---|
| 1801 | AGTTGGGCAA | ACGGGCTTTA | TATAGCCTGT | CCCCTCCACA | GCCCTAGTGC |
| | TCAACCCGTT | TGCCCGAAAT | ATATCGGACA | GGGGAGGTGT | CGGGATCACG |

------------------------------------------------------

ie-0 pr

| | | | | | |
|---|---|---|---|---|---|
| 1851 | CGTGCGCAAA | GTGCCTACGT | GACCAGGCTC | TCCTACGCAT | ATACAATCTT |
| | GCACGCGTTT | CACGGATGCA | CTGGTCCGAG | AGGATGCGTA | TATGTTAGAA |

------------------------------------------------------

ie-0 pr

| | | | | | |
|---|---|---|---|---|---|
| 1901 | ATCTCTATAG | ATAAGGTTTC | CATATATAAA | GCCTCTCGAT | GGCTGAACGT |
| | TAGAGATATC | TATTCCAAAG | GTATATATTT | CGGAGAGCTA | CCGACTTGCA |

------------------------------------------------------

ie-0 pr

| | | | | | |
|---|---|---|---|---|---|
| 1951 | GCACAGTATC | GTGTTGATTT | CTGAGTGCTA | ACTAACAGTT | ACAATGAACC |
| | CGTGTCATAG | CACAACTAAA | GACTCACGAT | TGATTGTCAA | TGTTACTTGG |

------------------------------------------------------

ie-0 pr

| | | | | | |
|---|---|---|---|---|---|
| 2001 | GTTTTTTTCG | AGAGAATAAC | ATTTTTGACG | CGCCAAGGAC | CGGGGGCAAG |
| | CAAAAAAAGC | TCTCTTATTG | TAAAAACTGC | GCGGTTCCTG | GCCCCCGTTC |

------------------------------------------------------

ie-0 pr

| | | | | | |
|---|---|---|---|---|---|
| 2051 | GGTCGTGCCA | AATCTTTGCC | AGCGCCTGCC | GCCAACTCGC | CGCCGTCGCC |
| | CCAGCACGGT | TTAGAAACGG | TCGCGGACGG | CGGTTGAGCG | GCGGCAGCGG |

------------------------------------------------------

ie-0 pr

| | | | | | |
|---|---|---|---|---|---|
| 2101 | TGTTCGTCCG | CCGCCAAAAT | CTAACATCAA | ACCACCTACG | CGCATCTCTC |
| | ACAAGCAGGC | GGCGGTTTTA | GATTGTAGTT | TGGTGGATGC | GCGTAGAGAG |

------------------------------------------------------

ie-0 pr

| | | | | | |
|---|---|---|---|---|---|
| 2151 | CGCCTAAACA | GCCTATGTGC | ACCTCTCCGG | CCAAGCCGTT | GGAGCACAGC |
| | GCGGATTTGT | CGGATACACG | TGGAGAGGCC | GGTTCGGCAA | CCTCGTGTCG |

------------------------------------------------------

ie-0 pr

| | | | | | |
|---|---|---|---|---|---|
| 2201 | AGCATTGTAA | GTAAAAAACC | AGTCGTCAAC | AGAAAAGATG | GATATTTTGT |
| | TCGTAACATT | CATTTTTTGG | TCAGCAGTTG | TCTTTTCTAC | CTATAAAACA |

------------------------------------------------------

ie-0 pr

| | | | | | |
|---|---|---|---|---|---|
| 2251 | GCCGCCCGAG | TTTGGGAACA | AGTTTGAAGG | TTTGCCCGCG | TACAGCGACA |
| | CGGCGGGCTC | AAACCCTTGT | TCAAACTTCC | AAACGGGCGC | ATGTCGCTGT |

------------------------------------------------------

ie-0 pr

p10

pr

--

| | | | | | |
|---|---|---|---|---|---|
| 2301 | AACTGGATTT | CAAACAAGAG | CGCGATCTAC | GTACCTGCAG | GCCCGGGCTC |
| | TTGACCTAAA | GTTTGTTCTC | GCGCTAGATG | CATGGACGTC | CGGGCCCGAG |

--------------------------------------------

```
          ie-0 pr
                    p10 pr
```

------------------------------------------------------

| | | | | | |
|---|---|---|---|---|---|
| 2351 | AACCCAACAC | AATATATTAT | AGTTAAATAA | GAATTATTAT | CAAATCATTT |
| | TTGGGTTGTG | TTATATAATA | TCAATTTATT | CTTAATAATA | GTTTAGTAAA |

p10 pr

---------------------------------------------

| | | | | | |
|---|---|---|---|---|---|
| 2401 | GTATATTAAT | TAAAATACTA | TACTGTAAAT | TACATTTTAT | TTACAATTCA |
| | CATATAATTA | ATTTTATGAT | ATGACATTTA | ATGTAAAATA | AATGTTAAGT |

lacZ

```
              -----------------------------------------------
                M T M I T D S L A V V L Q R R .
```

| | | | | | |
|---|---|---|---|---|---|
| 2451 | CTCTAGAATG | ACCATGATTA | CGGATTCACT | GGCCGTCGTT | TTACAACGTC |
| | GAGATCTTAC | TGGTACTAAT | GCCTAAGTGA | CCGGCAGCAA | AATGTTGCAG |

lacZ

------------------------------------------------------
. D W E N P G V T Q L N R L A A H

| | | | | | |
|---|---|---|---|---|---|
| 2501 | GTGACTGGGA | AAACCCTGGC | GTTACCCAAC | TTAATCGCCT | TGCAGCACAT |
| | CACTGACCCT | TTTGGGACCG | CAATGGGTTG | AATTAGCGGA | ACGTCGTGTA |

lacZ

------------------------------------------------------
P P F A S W R N S E E A R T D R P .

| | | | | | |
|---|---|---|---|---|---|
| 2551 | CCCCCTTTCG | CCAGCTGGCG | TAATAGCGAA | GAGGCCCGCA | CCGATCGCCC |
| | GGGGGAAAGC | GGTCGACCGC | ATTATCGCTT | CTCCGGGCGT | GGCTAGCGGG |

lacZ

------------------------------------------------------
. S Q Q L R S L N G E W R F A W F P .

| | | | | | |
|---|---|---|---|---|---|
| 2601 | TTCCCAACAG | TTGCGCAGCC | TGAATGGCGA | ATGGCGCTTT | GCCTGGTTTC |
| | AAGGGTTGTC | AACGCGTCGG | ACTTACCGCT | TACCGCGAAA | CGGACCAAAG |

lacZ

------------------------------------------------------

```
        Bsu36I
        ------
```

. A P E A V P E S W L E C D L P E

| | | | | | |
|---|---|---|---|---|---|
| 2651 | CGGCACCAGA | AGCGGTGCCG | GAAAGCTGGC | TGGAGTGCGA | TCTTCCTGAG |
| | GCCGTGGTCT | TCGCCACGGC | CTTTCGACCG | ACCTCACGCT | AGAAGGACTC |

lacZ

------------------------------------------------------
Bsu36I
-
A D T V V V P S N W Q M H G Y D A .

| | | | | | |
|---|---|---|---|---|---|
| 2701 | GCCGATACTG | TCGTCGTCCC | CTCAAACTCG | CAGATGCACG | GTTACGATGC |
| | CGGCTATGAC | AGCAGCAGGG | GAGTTTCACC | GTCTACGTGC | CAATGCTACG |

lacZ

------------------------------------------------------
. P I Y T N V T Y P I T V N P P F V .

| | | | | | |
|---|---|---|---|---|---|
| 2751 | GCCCATCTAC | ACCAACCTAA | CCTATCCCAT | TACGGTCAAT | CCGCCGTTTG |
| | CGGGTAGATG | TGGTTGCATT | GGATAGGGTA | ATGCCAGTTA | GGCGGCAAAC |

lacZ

------------------------------------------------------
. P T E N P T G C Y S L T F N V D

| | | | | | |
|---|---|---|---|---|---|
| 2801 | TTCCCACCGA | GAATCCGACG | GGTTGTTACT | CGCTCACATT | TAATGTTGAT |
| | AAGGGTGCCT | CTTAGGCTGC | CCAACAATGA | GCGAGTGTAA | ATTACAACTA |

lacZ

------------------------------------------------------
E S W L Q E C Q T R I I F D G V N .

| | | | | | |
|---|---|---|---|---|---|
| 2851 | GAAAGCTGGC | TACAGGAAGG | CCAGACGCCA | ATTATTTTTG | ATGGCGTTAA |
| | CTTTCGACCG | ATGTCCTTCC | GGTCTGCCCT | TAATAAAAAC | TACCCCAATT |

lacZ

------------------------------------------------------
. S A F H L W C N G R W V G Y G Q D .

| | | | | | |
|---|---|---|---|---|---|
| 2901 | CTCGGCGTTT | CATCTGTGGT | GCAACGGGCG | CTGGGTCGGT | TACCGCCAGG |
| | GAGCCGCAAA | GTAGACACCA | CCTTGCCCCC | CACCCAGCCA | ATCCCCCTCC |

lacZ

------------------------------------------------------
. S R L P S E F D L S A F L R A G

| | | | | | |
|---|---|---|---|---|---|
| 2951 | ACACTCGTTT | GCCGTCTGAA | TTTGACCTCA | GCCCATTTTT | ACGCGCCGGA |
| | TCTCACCAAA | CGCCAGACTT | AAACTGGACT | CCCGTAAAAA | TCCGCGGCCT |

lacZ

------------------------------------------------------
E N R L A V M V L R W S D G S Y L .

| | | | | | |
|---|---|---|---|---|---|
| 3001 | GAAAACCCCC | TCCCCCTCAT | GCTGCTCCCT | TCCAGTCACC | CCAGTTATCT |
| | CTTTTGGCCC | AGCGCCACTA | CCACCACCCA | ACCTCACTCC | CCTCAATACA |

lacZ

------------------------------------------------------
. E D Q D M W R M S G I F R D V S L .

| | | | | | |
|---|---|---|---|---|---|
| 3051 | CCAAGATCAC | GATATCTCGC | CCATCACCGG | CATTTTCCCT | CACCTCTCGT |
| | CCTTCTACTC | CTATACACCG | CCTACTCCCC | GTAAAAGGCA | CTCCACACCA |

lacZ

------------------------------------------------------
. L H K P T T Q I S D F H V A T R

| | | | | | |
|---|---|---|---|---|---|
| 3101 | TCCTCCATAA | ACCGACTACA | CAAATCAGCC | ATTTCCATCT | TCCCACTCGC |
| | ACCACCTATT | TGCCTCATCT | GTTTACTCCC | TAAACCTACA | ACCGTCAGCC |

lacZ

------------------------------------------------------
F N D D F S R A V L E A E V Q M C .

| | | | | | |
|---|---|---|---|---|---|
| 3151 | TTTAATGATG | ATTTCAGCCG | CGCTGTACTG | GAGGCTGAAG | TTCAGATGTG |
| | AAATTACTAC | TAAAGTCGGC | GCGACATGAC | CTCCGACTTC | AAGTCTACAC |

lacZ

------------------------------------------------------
. G E L R D Y L R V T V S L W Q G E .

| | | | | | |
|---|---|---|---|---|---|
| 3201 | CGGCGAGTTG | CGTGACTACC | TACGGGTAAC | AGTTTCTTTA | TGGCAGGGTG |
| | GCCGCTCAAC | GCACTGATGG | ATGCCCATTG | TCAAAGAAAT | ACCGTCCCAC |

lacZ

------------------------------------------------------
. T Q V A S G T A P F G G E I I D

| | | | | | |
|---|---|---|---|---|---|
| 3251 | AAACGCAGGT | CGCCAGCGGC | ACCGCGCCTT | TCGGCGGTGA | AATTATCGAT |
| | TTTGCGTCCA | GCGGTCGCCG | TGGCGCGGAA | AGCCGCCACT | TTAATAGCTA |

lacZ

------------------------------------------------------
E R G G Y A D R V T L R L N V E N .

| | | | | | |
|---|---|---|---|---|---|
| 3301 | GAGCGTGGTG | GTTATGCCGA | TCGCGTCACA | CTACGTCTGA | ACGTCGAAAA |
| | CTCGCACCAC | CAATACGGCT | AGCGCAGTGT | GATGCAGACT | TGCAGCTTTT |

lacZ

------------------------------------------------------
. P K L W S A E I P N L Y R A V V E .

| | | | | | |
|---|---|---|---|---|---|
| 3351 | CCCGAAACTG | TGGAGCGCCG | AAATCCCGAA | TCTCTATCGT | GCGGTGGTTG |
| | GGGCTTTGAC | ACCTCGCGGC | TTTAGGGCTT | AGAGATAGCA | CGCCACCAAC |

lacZ

------------------------------------------------------
. L H T A D G T L I E A E A C D V

| | | | | | |
|---|---|---|---|---|---|
| 3401 | AACTGCACAC | CGCCGACGGC | ACGCTGATTG | AAGCAGAAGC | CTGCGATGTC |
| | TTGACGTGTG | GCGGCTGCCG | TGCGACTAAC | TTCGTCTTCG | GACGCTACAG |

lacZ

------------------------------------------------------
G F R E V R I E N G L L L L N G K .

| | | | | | |
|---|---|---|---|---|---|
| 3451 | GGTTTCCGCG | AGGTGCGGAT | TGAAAATGGT | CTGCTGCTGC | TGAACGGCAA |
| | CCAAAGGCGC | TCCACCCCTA | ACTTTTACCA | GACGACGACG | ACTTGCCGTT |

lacZ

------------------------------------------------------
. P L L I R G V N R H E H H P L H G .

| | | | | | |
|---|---|---|---|---|---|
| 3501 | GCCGTTGCTG | ATTCCAGGCG | TTAACCGTCA | CGAGCATCAT | CCTCTGCATG |
| | CCGCAACCAC | TAAGCTCCGC | AATTGGCACT | CCTCGTACTA | GGAGACGTAC |

lacZ

------------------------------------------------------
. Q V M D E Q T M V Q D I L L M K

| | | | | | |
|---|---|---|---|---|---|
| 3551 | GTCAGGTCAT | CGATGACCAC | ACGATGGTGC | ACGATATCCT | GCTGATGAAG |
| | CAGTCCAGTA | CCTACTCGTC | TGCTACCACG | TCCTATAGGA | CGACTACTTC |

lacZ

------------------------------------------------------
Q N N F N A V R C S H Y P N H P L .

| | | | | | |
|---|---|---|---|---|---|
| 3601 | CAGAACAACT | TTAACGCCGT | GCGCTGTTCG | CATTATCCGA | ACCATCCGCT |
| | GTCTTGTTGA | AATTGCGGCA | CGCGACAAGC | GTAATAGGCT | TGGTAGGCGA |

lacZ

------------------------------------------------------
. W Y T L C D R Y G L Y V V D E A N .

| | | | | | |
|---|---|---|---|---|---|
| 3651 | GTGGTACACG | CTGTGCGACC | GCTACGGCCT | GTATGTGGTG | GATGAAGCCA |
| | CACCATGTGC | GACACGCTGG | CGATGCCGGA | CATACACCAC | CTACTTCGGT |

lacZ

------------------------------------------------------
. I E T H G M V P M N R L T D D P

| | | | | | |
|---|---|---|---|---|---|
| 3701 | ATATTGAAAC | CCACGGCATG | GTGCCAATGA | ATCGTCTGAC | CGATGATCCG |
| | TATAACTTTG | GGTGCCGTAC | CACGGTTACT | TAGCAGACTG | GCTACTAGGC |

lacZ

------------------------------------------------------
R W L P A M S E R V T R M V Q R D .

| | | | | | |
|---|---|---|---|---|---|
| 3751 | CGCTGGCTAC | CGGCGATGAG | CGAACGCGTA | ACGCGAATGG | TGCAGCGCGA |
| | GCGACCGATG | GCCGCTACTC | GCTTGCGCAT | TGCGCTTACC | ACGTCGCGCT |

lacZ

------------------------------------------------------
. R N H P S V I I W S L G N E S G H .

| | | | | | |
|---|---|---|---|---|---|
| 3801 | TCGTAATCAC | CCGAGTGTGA | TCATCTGGTC | GCTGGGGAAT | GAATCAGGCC |
| | AGCATTAGTG | GGCTCACACT | AGTAGACCAG | CGACCCCTTA | CTTAGTCCGG |

lacZ

------------------------------------------------------
. G A N H D A L Y R W I K S V D P

| | | | | | |
|---|---|---|---|---|---|
| 3851 | ACGGCGCTAA | TCACGACGCG | CTGTATCGCT | GGATCAAATC | TGTCGATCCT |
| | TGCCGCGATT | AGTGCTGCGC | GACATAGCGA | CCTAGTTTAG | ACAGCTAGGA |

lacZ

------------------------------------------------------
S R P V Q Y E G G G A D T T A T D .

| | | | | | |
|---|---|---|---|---|---|
| 3901 | TCCCGCCCGG | TGCAGTATGA | AGGCGGCGGA | GCCGACACCA | CGGCCACCGA |
| | AGGGCGGGCC | ACGTCATACT | TCCGCCGCCT | CGGCTGTGGT | GCCGGTGGCT |

lacZ

------------------------------------------------------
. I I C P M Y A R V D E D Q P F P A .

| | | | | | |
|---|---|---|---|---|---|
| 3951 | TATTATTTGC | CCGATGTACG | CGCGCGTGGA | TGAAGACCAG | CCCTTCCCGG |
| | ATAATAAACG | GGCTACATGC | GCGCGCACCT | ACTTCTGGTC | GGGAAGGGCC |

lacZ

------------------------------------------------------
. V P K W S I K K W L S L P G E T

| | | | | | |
|---|---|---|---|---|---|
| 4001 | CTGTGCCGAA | ATGGTCCATC | AAAAAATGGC | TTTCGCTACC | TGGAGAGACG |
| | GACACGGCTT | TACCAGGTAG | TTTTTTACCG | AAAGCGATGG | ACCTCTCTGC |

lacZ

------------------------------------------------------
R P L I L C E Y A H A M G N S L G .

| | | | | | |
|---|---|---|---|---|---|
| 4051 | CGCCCGCTGA | TCCTTTGCGA | ATACGCCCAC | GCGATGGGTA | ACAGTCTTGG |
| | GCGGGCGACT | AGGAAACGCT | TATGCGGGTG | CGCTACCCAT | TGTCAGAACC |

lacZ

------------------------------------------------------
. G F A K Y W Q A F R Q Y P R L Q G .

| | | | | | |
|---|---|---|---|---|---|
| 4101 | CGGTTTCGCT | AAATACTGGC | AGGCGTTTCG | TCAGTATCCC | CGTTTACAGG |
| | GCCAAAGCGA | TTTATGACCG | TCCGCAAAGC | AGTCATAGGG | GCAAATGTCC |

lacZ

------------------------------------------------------
. G F V W D W V D Q S L I K Y D E

| | | | | | |
|---|---|---|---|---|---|
| 4151 | GCGGCTTCGT | CTGGGACTGG | GTGGATCAGT | CGCTGATTAA | ATATGATGAA |
| | CGCCGAAGCA | GACCCTGACC | CACCTAGTCA | GCGACTAATT | TATACTACTT |

lacZ

------------------------------------------------------
N G N P W S A Y G G D F G D T P N .

| | | | | | |
|---|---|---|---|---|---|
| 4201 | AACGGCAACC | CGTGGTCGGC | TTACGGCGGT | GATTTTGGCG | ATACGCCGAA |
| | TTGCCGTTGG | GCACCAGCCG | AATGCCGCCA | CTAAAACCGC | TATGCGGCTT |

lacZ

------------------------------------------------------
. D R Q F C M N G L V F A D R T P H .

| | | | | | |
|---|---|---|---|---|---|
| 4251 | CGATCGCCAG | TTCTGTATGA | ACGGTCTGGT | CTTTGCCGAC | CGCACGCCGC |
| | GCTAGCGGTC | AAGACATACT | TGCCAGACCA | GAAACGGCTG | GCGTGCGGCG |

lacZ

------------------------------------------------------
. P A L T E A K H Q Q Q F F Q F R

| | | | | | |
|---|---|---|---|---|---|
| 4301 | ATCCAGCGCT | GACGGAAGCA | AAACACCAGC | AGCAGTTTTT | CCAGTTCCGT |
| | TAGGTCGCGA | CTGCCTTCGT | TTTGTGGTCG | TCGTCAAAAA | GGTCAAGGCA |

lacZ

------------------------------------------------------
L S G Q T I E V T S E Y L F R H S .

| | | | | | |
|---|---|---|---|---|---|
| 4351 | TTATCCGGGC | AAACCATCGA | AGTGACCAGC | GAATACCTCT | TCCGTCATAG |
| | AATAGGCCCG | TTTCGTACCT | TCACTGGTCC | CTTATGGACA | AGGCAGTATC |

lacZ

------------------------------------------------------
. D N E L L H W M V A L D G K P L A .

| | | | | | |
|---|---|---|---|---|---|
| 4401 | CGATAACGAG | CTCCTGCACT | GCATGGTGGC | CCTGGATGGT | AAGCCGCTGG |
| | GCTATTGCTC | GACCACGTGA | CCTACCACCG | CCACCTACCA | TTCGGCGACC |

lacZ

------------------------------------------------------
. S G E V P L D V A P Q G K Q L I

| | | | | | |
|---|---|---|---|---|---|
| 4451 | CAAGCGGTGA | AGTGCCTCTG | GATGTCGCTC | CACAAGGTAA | ACAGTTGATT |
| | GTTCGCCACT | TCACGGAGAC | CTACAGCGAG | GTGTTCCATT | TGTCAACTAA |

lacZ

------------------------------------------------------
E L P E L P Q P E S A G Q L W L T .

| | | | | | |
|---|---|---|---|---|---|
| 4501 | GAACTGCCTG | AACTACCGCA | GCCGGAGAGC | GCCGGGCAAC | TCTGGCTCAC |
| | CTTGACGGAC | TTGATGGCGT | CGGCCTCTCG | CGGCCCGTTG | AGACCGAGTG |

lacZ

------------------------------------------------------
. V R V V Q P N A T A W S E A G H I .

| | | | | | |
|---|---|---|---|---|---|
| 4551 | AGTACGCGTA | GTGCAACCGA | ACGCGACCGC | ATGGTCAGAA | GCCGGGCACA |
| | TCATGCGCAT | CACGTTGGCT | TGCGCTGGCG | TACCAGTCTT | CGGCCCGTGT |

lacZ

------------------------------------------------------
. S A W Q Q W R L A E N L S V T L

| | | | | | |
|---|---|---|---|---|---|
| 4601 | TCAGCGCCTG | GCAGCAGTGG | CGTCTGGCGG | AAAACCTCAG | TGTGACGCTC |
| | AGTCGCGGAC | CGTCGTCACC | GCAGACCGCC | TTTTGGAGTC | ACACTGCGAG |

lacZ

------------------------------------------------------
P A A S H A I P H L T T S E M D F .

| | | | | | |
|---|---|---|---|---|---|
| 4651 | CCCGCCGCGT | CCCACGCCAT | CCCGCATCTG | ACCACCAGCG | AAATGGATTT |
| | GGGCGGCGCA | GGGTGCGGTA | GGGCGTAGAC | TGGTGGTCGC | TTTACCTAAA |

lacZ

------------------------------------------------------
. C I E L G N K R W Q F N R Q S G F .

| | | | | | |
|---|---|---|---|---|---|
| 4701 | TTGCATCGAG | CTGGGTAATA | AGCGTTGGCA | ATTTAACCGC | CAGTCAGGCT |
| | AACGTAGCTC | GACCCATTAT | TCGCAACCGT | TAAATTGGCG | GTCAGTCCGA |

lacZ

------------------------------------------------------
. L S Q M W I G D K K Q L L T P L

| | | | | | |
|---|---|---|---|---|---|
| 4751 | TTCTTTCACA | GATGTGGATT | GGCGATAAAA | AACAACTGCT | GACGCCGCTG |
| | AAGAAAGTGT | CTACACCTAA | CCGCTATTTT | TTGTTGACGA | CTGCGGCGAC |

lacZ

------------------------------------------------------
R D Q F T R A P L D N D I G V S E .

| | | | | | |
|---|---|---|---|---|---|
| 4801 | CGCGATCAGT | TCACCCGTGC | ACCGCTGGAT | AACGACATTG | GCGTAAGTGA |
| | GCGCTAGTCA | AGTGGGCACG | TGGCGACCTA | TTGCTGTAAC | CGCATTCACT |

lacZ

------------------------------------------------------
. A T R I D P N A W V E R W K A A G .

| | | | | | |
|---|---|---|---|---|---|
| 4851 | AGCGACCCGC | ATTGACCCTA | ACGCCTGGGT | CGAACGCTGG | AAGGCGGCGG |
| | TCGCTGGGCG | TAACTGGGAT | TGCGGACCCA | GCTTGCGACC | TTCCGCCGCC |

lacZ

------------------------------------------------------
. H Y Q A E A A L L Q C T A D T L

| | | | | | |
|---|---|---|---|---|---|
| 4901 | GCCATTACCA | GGCCGAAGCA | GCGTTGTTGC | AGTGCACGGC | AGATACACTT |
| | CGGTAATGGT | CCGGCTTCGT | CGCAACAACG | TCACGTGCCG | TCTATGTGAA |

lacZ

------------------------------------------------------
A D A V L I T T A H A W Q H Q G K .

| | | | | | |
|---|---|---|---|---|---|
| 4951 | CCTGATGCGG | TCCTGATTAC | GACCGCTCAC | GCGTGGCACC | ATCAGGGGAA |
| | CGACTACGCC | ACGACTAATG | CTGCCGAGTG | CGCACCGTCG | TAGTCCCCTT |

lacZ

------------------------------------------------------
. T L F I S R K T Y R I D G S G Q M .

| | | | | | |
|---|---|---|---|---|---|
| 5001 | AACCTTATTT | ATCAGCCGGA | AAACCTACCG | GATTGATCGT | AGTGGTCAAA |
| | TTGCAATAAA | TAGTCGGCCT | TTTGGATGGC | CTAACTACCA | TCACCAGTTT |

lacZ

------------------------------------------------------
. A I T V D V E V A S D T P H P A

| | | | | | |
|---|---|---|---|---|---|
| 5051 | TCGCGATTAC | CGTTGATGTT | GAAGTGGCGA | CCGATACACC | GCATCCGGCG |
| | ACCGCTAATG | GCAACTACAA | CTTCACCGCT | CGCTATGTGG | CGTAGCCCGC |

lacZ

------------------------------------------------------
R I G L N C Q L A Q V A E R V N W .

| | | | | | |
|---|---|---|---|---|---|
| 5101 | CGGATTGGCC | TGAACTGCCA | CCTGGCGCAG | GTAGCAGAGC | GGGTAAACTG |
| | GCCTAACCGG | ACTTGACGGT | CGACCGCGTC | CATCGTCTCG | CCCATTTGAC |

lacZ

------------------------------------------------------
. L G L G P Q E N Y P D R L T A A C .

| | | | | | |
|---|---|---|---|---|---|
| 5151 | GCTCGGATTA | GGCCCGCAAG | AAAACTATCC | CGACCGCCTT | ACTGCCGCCT |
| | CGAGCCTAAT | CCCGGCGTTC | TTTTGATAGC | GCTGGCGGAA | TGACGGCCGA |

lacZ

------------------------------------------------------
. F D R W D L P L S D M Y T P Y V

| | | | | | |
|---|---|---|---|---|---|
| 5201 | GTTTTGACCG | CTCGGATCTG | CCATTGTCAG | ACATGTATAC | CCCGTACGTC |
| | CAAAACTCGC | CACCGTAGAC | GGTAACACTC | TGTACATATG | GGGCATGCAG |

lacZ

------------------------------------------------------
F P S E N G L R C G T R E L N Y G .

| | | | | | |
|---|---|---|---|---|---|
| 5251 | TTCCCGAGCC | AAAACCGTCT | GCGCTGCGGG | ACGCCCGAAT | TGAATTATGG |
| | AAGGGCTCGC | TTTTGCCAGA | CCCCACGCCC | TGCGCGCTTA | ACTTAATACC |

lacZ

------------------------------------------------------
. P H Q W R G D F Q F N I S R Y S Q .

| | | | | | |
|---|---|---|---|---|---|
| 5301 | CCCACACCAG | TGGCGCGGCG | ACTTCCACTT | CAACATCAGC | CGCTACAGTC |
| | GGGTGTCGTC | ACCCCGCCCC | TCAAGCTCAA | GTTGTAGTCC | GCGATGTCAG |

lacZ

------------------------------------------------------
. Q Q L M E T S H R H L L H A E E

| | | | | | |
|---|---|---|---|---|---|
| 5351 | AACACCAACT | GATCCAAACC | ACCCATCCCC | ATCTCCTCCA | CCCCCAACAA |
| | TTGTCCTTCA | CTACCTTTGG | TCCGTACCGC | TAGACGACCT | GCCCCTTCTT |

lacZ

------------------------------------------------------
G T W L N I D G F H M G I G G D D .

| | | | | | |
|---|---|---|---|---|---|
| 5401 | GGCACATGGC | TGAATATCGA | CGGTTTCCAT | ATGGGGATTG | GTGGCGACGA |
| | CCGTGTACCG | ACTTATAGCT | GCCAAAGGTA | TACCCCTAAC | CACCGCTGCT |

lacZ

------------------------------------------------------
. S W S P S V S A E F Q L S A G R Y .

| | | | | | |
|---|---|---|---|---|---|
| 5451 | CTCCTGGAGC | CCGTCAGTAT | CGGCGGAATT | CCAGCTGAGC | GCCGGTCGCT |
| | GAGGACCTCG | GGCAGTCATA | GCCGCCTTAA | GGTCGACTCG | CGGCCAGCGA |

| | |
|---|---|
| lacZ | AttR2 |

| | |
|---|---|
| ------------------------------- | --- |
| . H Y Q L V W C Q K | |

| | | | | | |
|---|---|---|---|---|---|
| 5501 | ACCATTACCA | GTTGGTCTGG | TGTCAAAAAT | AATGACTGCA | GGTCGACCAT |
| | TGGTAATGGT | CAACCAGACC | ACAGTTTTTA | TTACTGACGT | CCAGCTGGTA |

AttR2

------------------------------------------------------

| | | | | | |
|---|---|---|---|---|---|
| 5551 | AGTGACTGGA | TATGTTGTGT | TTTACAGTAT | TATGTAGTCT | GTTTTTTATG |
| | TCACTGACCT | ATACAACACA | AAATGTCATA | ATACATCAGA | CAAAAAATAC |

AttR2

------------------------------------------------------

| | | | | | |
|---|---|---|---|---|---|
| 5601 | CAAAATCTAA | TTTAATATAT | TGATATTTAT | ATCATTTTAC | GTTTCTCGTT |
| | GTTTTAGATT | AAATTATATA | ACTATAAATA | TAGTAAAATG | CAAAGAGCAA |

| | |
|---|---|
| AttR2 | V5/His |
| ------------------------ | --------- |

G K P

| | | | | | |
|---|---|---|---|---|---|
| 5651 | CAGCTTTCTT | GTACAAAGTG | GTGAGAATGA | ATGAAGATCT | GGGGAAGCCT |
| | GTCGAAAGAA | CATGTTTCAC | CACTCTTACT | TACTTCTAGA | CCCCTTCGGA |

V5/His

------------------------------------------------------
I P N P L L G L D S T R T G H H H .

| | | | | | |
|---|---|---|---|---|---|
| 5701 | ATCCCTAACC | CTCTCCTCGG | TCTCGATTCT | ACGCGTACCG | GTCATCATCA |
| | TAGGGATTGG | GAGAGGAGCC | AGAGCTAAGA | TGCGCATGGC | CAGTAGTAGT |

stop codon
---

V5/His
-----------
. H H H

| | | |
|---|---|---|
| 5751 | CCATCACCAT | TGA |
| | GGTAGTGGTA | ACT |

TABLE 15
Baculoviral promoter sequences.

AcMNPV ORF 25 promoter sequence
Ggtgtcttcattagtatgccaatcacgtacgcaacagtcgcaaaagaaacacacagtttcgtctccgcgacccgtgtaaaaaagtcccg
cttccgcaatgtttgtaatcatgtcacgcaatgcggcaggccaaaagttaacaaacgtatccatacgcgactgtaaattggacatgcatct
gtacacacacttgggtttgccttctttcactagtacagcgttgatggtaatgttgtcgccaaacgattcacgctcggcgatcttgttagcata
cgcgcaatacggcgacaaggttacgtgtgcatattcaatacactcgtcttcggaccaattttttatttctgcttcgcaatactcgcacacaa
cgtgatcgtcaacttgattgtatttaaacccgttaacgatcaagctgttaataaacgccgtgttttcaatgggataattttcaaacgaactatg
tctttctattaacatgtcgaatacgtgttcggcggtgttgtcgcgaaagttgtcacacacgctgataaaataaaacgggggcgtgtcctcg
ttcattttagctcgttaaagttacggtcaaaatgagcacgtttgcgtcgttttggtttagcgacacgtttatatggcccagtttggtttttgtttc
ggcgttaatgacgtgcactgtggacaaatcgtgttctaaaactacaaactcgtactcgaaaatgtttgatatgtagttggttagccgatcta
tcttaaaattaaacttttgcaactcgctgatagagcacacgtccacatacttgtcgataaacccgttgctcaaccgcttcaaaacggtgtaa
ttttgtagcttgaaaggggcgcatttggaatgactaaaaggaatatttttcaataaatcgtcagtagtgtacgcaaacgcgttgtctacgca
catgctggcaacagagtcgtccatatttattatatatcttatattctgtgaaacacttcaattagacttgaaccacagcagacagcgcacgt
cggtagc
AcMNPV lef 3 promoter
Ccgagaagaaggcggtttgtataaaacccatttttcgaaatggttaacaaacttgtttagcatttggatcgtttcgtgttcaaacgcgtcga
aaacttttaaaacgcaattgccgccgggacgcaggcaaattaaaattagctgcgtctcgcacatgatcaaatcaaagttgagacgttctt
gttcgttttcgcgtccattaacgtcaaccgagccatctgccaacaccagatcgcacgcgttgccacacttgatgctaatctcaaatacaac
atttttatcaaacacgtcgcctgacttgtcgggccccgtaatggttgtgaaatttttgcgtttgcgcactgtcggtttgtacacgcacaccga
gttgtttgtcaacgtgacgccatacgctttgcaaagcgggttcaacgacatggtatagttggcaaactcgcccggtccgccgcacaaat
ccaaaaacgtgtcaacgtgtcggcaaacgtgaaactttttgtcgatctctgatagttttcgccaacatctaggtctgcgcgttgggcgtttg
tcaaataattttgagcgagcgcaaaccaccgacttgctgctgaacgtgttcaaaccatctttgagtttatttaatttttgctgcaacatttttac
tcttcgtgtcggtcgcaatgtttgtgtcgaaaaagacggccaacacgctcagcaaaactatacaaataaagaacaaaaatacgtacgca
atattaacattgaccgtttgatcgttaaatcggacgggtctgttcagagccgctcttattctctcgttgtacattgttaaagtttttgtttttaaatt
gtacacaatcggcgtgttgtagtcgaaattttcaaaatcggctttttgaaacattgttctgaacgtgttgtcgagcggcgtgttgctggcca
cgtttataatcaactccctccacgctaacgaacggtgctctggcgacacttcgatttcgtcgccattcagtatttgccatcggatagattcc
cacatatcgacaacagcaat
AcMNPV TLP promoter
tgctagcccaattggccactgttgtacgaaatatcgtcgtcaacgtgtttgaatacatgttggcccgtaccgttgggtaaatctatgcatct
ggagtcgccggaacactcgtactggttgtcagagtttctgatccggttgatgcacgttatcagttgtgactcgttattattcaaacatttgaa
atattgcgtgtcgccgatatcggccgttatgtacgtgtgtccggcgccgttaaacgcgcacggatgcgcttccacgcacgacattaagtt
gcgatcaaatattttattcgcggggcattcgcccaccacgtggcgcccatttacgcactgcataaactggttgacgagcaaattggagg
gaaagtatgatagtatatagccgtctggcctgttttcacacaattcgttaactttacactggccggtttccgcgtcaaacgtgtaattatctg
gacattcttcgactgcgtgcgctccgtttgcaaaacacctaagatagaacgtgggatgatacaagtgcgcgttggtagaataatctttgtc
caagtgttggttcaacaccaacgtgtccagcaaacgctcgtccatgggataaagaccggcagacttgttgtcgcacggcggcacggg
aacacattttagttgtgcgtaatcaaagttaaaatatgcggggcatttcatggtcacgtcggccttgtcgccgctcaaaataaactcgttgg
gattttcatcatttgctctaacgcgatcgtgtacgattcgatcaacaggttgaaatttttgatttaagaaatcaaaaatttcaatccggtcatca
tgcacgctttcgtgataggtggaaaggtcgacggtgttgaaccacgttacaatataagtgttttgcataatatccgacacgtagcctattac
gtcgggtgtgggttcgtctgcgttggtgcgcttcacatattcagtcatcacttggagccgcttggtgaaagtcgtttcgtcaaattcaaaat
aaattgccaaatacattaaagtaaacgctattataagaaaaaagctt
AcMNPV hr5 sequence
Gttttacgcgtagaattctacccgtaaagcgagtttagttatgagccatgtgcaaaacatgacatcagcttttatttttataacaaatgacat
catttcttgattgtgttttacacgtagaattctactcgtaaagcgagttcagttttgaaaaacaaatgacatcatctttttgattgtgctttacaa
gtagaattctacccgtaaatcaagttcggttttgaaaaacaaatgagtcatattgtatgatatcatattgcaaacaaatgactcatcaatcga
tcgtgcgtacacgtagaattctactcgtaaagcgagtttatgagccgtgtgcaaaacatgacatcatctcgatttgaaaaacaaatgacat
catccactgatcgtgcgttacaagtagaattctactcgtaaagccagttcggttatgagccgtgtgcaaaacatgacatcagcttatgact
cgtacttgattgtgttttacgcgtagaattctactcgtaaagc

TABLE 16
IE-1 promoter, coding, and polypeptide sequence.

AcMNPV IE-1 promoter
Gttttacgcgtagaattctacccgtaaagcgagtttagttatgagccatgtgcaaaacatgacatcagcttttatttttataacaaatgacat
catttcttgattgtgttttacacgtagaattctactcgtaaagcgagttcagttttgaaaaacaaatgacatcatctttttgattgtgctttacaa
gtagaattctacccgtaaatcaagttcggttttgaaaaacaaatgagtcatattgtatgatatcatattgcaaacaaatgactcatcaatcga
tcgtgcgtacacgtagaattctactcgtaaagcgagtttatgagccgtgtgcaaaacatgacatcatctcgatttgaaaaacaaatgacat
catccactgatcgtgcgttacaagtagaattctactcgtaaagccagttcggttatgagccgtgtgcaaaacatgacatcagcttatgact
cgtacttgattgtgttttacgcgtagaattctactcgtaaagc
AcMPNV IE-1 coding sequence
atgacgcaaattaattttaacgcgtcgtacaccagcgcttcgacgccgtcccgagcgtcgttcgacaacagctattcagagttttgtgata
aacaacccaacgactatttaagttattataaccatcccaccccggatggagccgacacggtgatatctgacagcgagactgcggcagc
ttcaaactttttggcaagcgtcaactcgttaactgataatgatttagtggaatgtttgctcaagaccactgataatctcgaagaagcagttag
ttctgcttattattcggaatcccttgagcagcctgttgtggagcaaccatcgcccagttctgcttatcatgcggaatcttttgagcattctgct
ggtgtgaaccaaccatcggcaactggaactaaacggaagctggacgaatacttggacaattcacaaggtgtggtgggccagtttaac
aaaattaaattgaggcctaaatacaagaaaagcacaattcaaagctgtgcaacccttgaacagacaattaatcacaacacgaacatttg
cacggtcgcttcaactcaagaaattacgcattattttactaatgattttgcgccgtatttaatgcgtttcgacgacaacgactacaattccaa
caggttctccgaccatatgtccgaaactggttattacatgtttgtggttaaaaaaagtgaagtgaagccgtttgaaattatatttgccaagta
cgtgagcaatgtggtttacgaatatacaaacaattattacatggtagataatcgcgtgtttgtggtaacttttgataaaattaggtttatgattt
cgtacaatttggttaaagaaaccggcatagaaattcctcattctcaagatgtgtgcaacgacgagacggctgcacaaaattgtaaaaaat
gccatttcgtcgatgtgcaccacacgtttaaagctgctctgacttcatattttaatttagatatgtattacgcgcaaaccacatttgtgactttg
ttacaatcgttgggcgaaagaaaatgtgggtttcttttgagcaagttgtacgaaatgtatcaagataaaaatttatttactttgcctattatgct
tagtcgtaaagagagtaatgaaattgagactgcatctaataatttctttgtatcgccgtatgtgagtcaaatattaaagtattcggaaagtgt
gcagtttcccgacaatcccccaaacaaatatgtggtggacaatttaaatttaattgttaacaaaaaaagtacgctcacgtacaaatacagc
agcgtcgctaatcttttgtttaataattataaatatcatgacaatattgcgagtaataataacgcagaaaatttaaaaaaggttaagaaggag
gacggcagcatgcacattgtcgaacagtatttgactcagaatgtagataatgtaaagggtcacaattttatagtattgtctttcaaaaacga
ggagcgattgactatagctaagaaaaacaaagagttttattggatttctggcgaaattaaagatgtagacgttagtcaagtaattcaaaaa
tataatagatttaagcatcacatgtttgtaatcggtaaagtgaaccgaagagagagcactacattgcacaataatttgttaaaattgttagct
ttaatattacagggtctggttccgttgtccgacgctataacgtttgcggaacaaaaactaaattgtaaatataaaaaattcgaatttaat
AcMNPV IE-1 protein sequence
Mtqinfnasytsastpsrasfdnsysefcdkqpndylsyynhptpdgadtvisdsetaaasnflasvnsltdndlvecllkttdnlee
avssayysesleqpvveqpspssayhaesfehsagvnqpsatgtkrkldeyldnsqgvvgqfnkiklrpkykkstiqscatleqti
nhntnictvastqeithyftndfapylmrfddndynsnrfsdhmsetgyymfvvkksevkpfeiifakyvsnvvyeytnnyym
vdnrvfvvtfdkirfmisynlvketgieiphsqdvcndetaaqnckkchfvdvhhtfkaaltsyfnldmyyaqttfvtllqslgerk
cgfllsklyemyqdknlftlpimlsrkesneietasnnffvspyvsqilkysesvqfpdnppnkyvvdnlnlivnkkstltykyssv
anllfnnykyhdniasnnnaenlkkvkkedgsmhiveqyltqnvdnvkghnfivlsfkneerltiakknkefywisgeikdvdv
sqviqkynrfkhhmfvigkvnrresttlhnnllkllalilqglvplsdaitfaeqklnckykkfefn

TABLE 17
Nucleotide sequence of plasmid pLenti6/V5-DEST.

AATGTAGTCTTATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTTATTAGGAAGGC
AACAGACGGGTCTGACATGGATTGGACGAACCACTGAATTGCCGCATTGCAGAGATATTGTATTTAAGTG
CCTAGCTCGATACATAAACGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCGGTTA
AGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCT
TCAGACAGGATCAGAAGAACTTAGATCATTATATAATACAGTAGCAACCCTCTATTGTGTGCATCAAAGG
ATAGAGATAAAAGACACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTGGAGAAGTGAATTAT
ATAAATATAAAGTAGTAAAAATTGAACCATTAGGAGTAGCACCCACCAAGGCAAAGAGAAGAGTGGTGCA
GAGAGAAAAAAGAGCAGTGGGAATAGGAGCTTTGTTCCTTGGGTTCTTGGGAGCAGCAGGAAGCACTATG
GGCGCAGCGTCAATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTGCTCTGGA
AAACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTGA
AGAATCGCAAAACCAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCATTATCGTT
TCAGACCCACCTCCCAACCCCGAGGGGACCCGACAGGCCCGAAGGAATAGAAGAAGAAGGTGGAGAGAGA
GACAGAGACAGATCCATTCGATTAGTGAACGGATCTCGACGGTATCGATAAGCTTGGGAGTTCCGCGTTA
CATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGTACATCTACGTATT
AGTCATCGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTGACCTCCAT
AGAAGACACCGACTCTAGAGGATCCACTAGTCCAGTGTGGTGGAATTCTGCAGATATCAACAAGTTTGTA
CAAAAAAGCTGAACGAGAAACGTAAAATGATATAAATATCAATATATTAAATTAGATTTTGCATAAAAAA
CAGACTACATAATACTGTAAAACACAACATATCCAGTCACTATGGCGGCCGCATTAGGCACCCCAGGCTT
TACACTTTATGCTTCCGGCTCGTATAATGTGTGGATTTTGAGTTAGGATCCGGCGAGATTTTCAGGAGCT
AAGGAAGCTAAAATGGAGAAAAAAATCACTGGATATACCACCGTTGATATATCCCAATGGCATCGTAAAG
AACATTTTGAGGCATTTCAGTCAGTTGCTCAATGTACCTATAACCAGACCGTTCAGCTGGATATTACGGC
CTTTTTAAAGACCGTAAAGAAAAATAAGCACAAGTTTTATCCGGCCTTTATTCACATTCTTGCCCGCCTG
ATGAATGCTCATCCGGAATTCCGTATGGCAATGAAAGACGGTGAGCTGGTGATATGGGATAGTGTTCACC
CTTGTTACACCGTTTTCCATGAGCAAACTGAAACGTTTTCATCGCTCTGGAGTGAATACCACGACGATTT
CCGGCAGTTTCTACACATATATTCGCAAGATGTGGCGTGTTACGGTGAAAACCTGGCCTATTTCCCTAAA
GGGTTTATTGAGAATATGTTTTTCGTCTCAGCCAATCCCTGGGTGAGTTTCACCAGTTTTGATTTAAACG
TGGCCAATATGGACAACTTCTTCGCCCCCGTTTTCACCATGGGCAAATATTATACGCAAGGCGACAAGGT
GCTGATGCCGCTGGCGATTCAGGTTCATCATGCCGTCTGTGATGGCTTCCATGTCGGCAGAATGCTTAAT
GAATTACAACAGTACTGCGATGAGTGGCAGGGCGGGGCGTAAAGATCTGGATCCGGCTTACTAAAAGCCA
GATAACAGTATGCGTATTTGCGCGCTGATTTTTGCGGTATAAGAATATATACTGATATGTATACCCGAAG
TATGTCAAAAAGAGGTGTGCTATGAAGCAGCGTATTACAGTGACAGTTGACAGCGACAGCTATCAGTTGC
TCAAGGCATATATGATGTCAATATCTCCGGTCTGGTAAGCACAACCATGCAGAATGAAGCCCGTCGTCTG
CGTGCCGAACGCTGGAAAGCGGAAAATCAGGAAGGGATGGCTGAGGTCGCCCGGTTTATTGAAATGAACG
GCTCTTTTGCTGACGAGAACAGGGACTGGTGAAATGCAGTTTAAGGTTTACACCTATAAAAGAGAGAGCC
GTTATCGTCTGTTTGTGGATGTACAGAGTGATATTATTGACACGCCCGGGCGACGGATGGTGATCCCCCT
GGCCAGTGCACGTCTGCTGTCAGATAAAGTCTCCCGTGAACTTTACCCGGTGGTGCATATCGGGGATGAA
AGCTGGCGCATGATGACCACCGATATGGCCAGTGTGCCGGTCTCCGTTATCGGGGAAGAAGTGGCTGATC
TCAGCCACCGCGAAAATGACATCAAAAACGCCATTAACCTGATGTTCTGGGGAATATAAATGTCAGGCTC
CGTTATACACAGCCAGTCTGCAGGTCGACCATAGTGACTGGATATGTTGTGTTTTACAGTATTATGTAGT
CTGTTTTTTATGCAAAATCTAATTTAATATATTGATATTTATATCATTTTACGTTTCTCGTTCAGCTTTC
TTGTACAAAGTGGTTGATATCCAGCACAGTGGCGGCCGCTCGAGTCTAGAGGGCCCGCGGTTCGAAGGTA
AGCCTATCCCTAACCCTCTCCTCGGTCTCGATTCTACGCGTACCGGTTAGTAATGAGTTTGGAATTAATT
CTGTGGAATGTGTGTCAGTTAGGGTGTGGAAAGTCCCCAGGCTCCCCAGGCAGGCAGAAGTATGCAAAGC
ATGCATCTCAATTAGTCAGCAACCAGGTGTGGAAAGTCCCCAGGCTCCCCAGCAGGCAGAAGTATGCAAA
GCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCT
GCCTCTGAGCTATTCCAGAAGTAGTGAGGAGGCTTTTTTGGAGGCCTAGGCTTTTGCAAAAAGCTCCCGG
GAGCTTGTATATCCATTTTCGGATCTGATCAGCACGTGTTGACAATTAATCATCGGCATAGTATATCGGC
ATAGTATAATACGACAAGGTGAGGAACTAAACCATGGCCAAGCCTTTGTCTCAAGAAGAATCCACCCTCA
TTGAAAGAGCAACGGCTACAATCAACAGCATCCCCATCTCTGAAGACTACAGCGTCGCCAGCGCAGCTCT
CTCTAGCGACGGCCGCATCTTCACTGGTGTCAATGTATATCATTTTACTGGGGGACCTTGTGCAGAACTC
GTGGTGCTGGGCACTGCTGCTGCTGCGGCAGCTGGCAACCTGACTTGTATCGTCGCGATCGGAAATGAGA
ACAGGGGCATCTTGAGCCCCTGCGGACGGTGCCGACAGGTGCTTCTCGATCTGCATCCTGGGATCAAAGC
CATAGTGAAGGACAGTGATGGACAGCCGACGGCAGTTGGGATTCGTGAATTGCTGCCCTCTGGTTATGTG
TGGGAGGGCTAAGCACAATTCGAGCTCGGTACCTTTAAGACCAATGACTTACAAGGCAGCTGTAGATCTT
AGCCACTTTTTAAAAGAAAAGGGGGGACTGGAAGGGCTAATTCACTCCCAACGAAGACAAGATCTGCTTT
TTGCTTGTACTGGGTCTCTCTGGTTAGACCAGATCTGAGCCTGGGAGCTCTCTGGCTAACTAGGGAACCC
ACTGCTTAAGCCTCAATAAAGCTTGCCTTGAGTGCTTCAAGTAGTGTGTGCCCGTCTGTTGTGTGACTCT
GGTAACTAGAGATCCCTCAGACCCTTTTAGTCAGTGTGGAAAATCTCTAGCAGTAGTAGTTCATGTCATC
TTATTATTCAGTATTTATAACTTGCAAAGAAATGAATATCAGAGAGTGAGAGGAACTTGTTTATTGCAGC
TTATAATGGTTACAAATAAAGCAATAGCATCACAAATTTCACAAATAAAGCATTTTTTTCACTGCATTCT
AGTTGTGGTTTGTCCAAACTCATCAATGTATCTTATCATGTCTGGCTCTAGCTATCCCGCCCCTAACTCC
GCCCATCCCGCCCCTAACTCCGCCCAGTTCCGCCCATTCTCCGCCCCATGGCTGACTAATTTTTTTTATT
TATGCAGAGGCCGAGGCCGCCTCGGCCTCTGAGCTATTCCAGAAGTAGTGAGGAGGCTTTTTTGGAGGCC
TAGGGACGTACCCAATTCGCCCTATAGTGAGTCGTATTACGCGCGCTCACTGGCCGTCGTTTTACAACGT
CGTGACTGGGAAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGCGAATGGGACGC
GCCCTGTAGCGGCGCATTAAGCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCGGTCTATTCTTTTG
ATTTATAAGGGATTTTGCCGATTTCGGCCTATTGGTTAAAAAATGAGCTGATTTAACAAAAATTTAACGC
GAATTTTAACAAAATATTAACGCTTACAATTTAGGTGGCACTTTTCGGGGAAATGTGCGCGGAACCCCTA
TTTGTTTATTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTGGGTG
CACGAGTGGGTTACATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGTGACACCACGA
TGCCTGTAGCAATGGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGGCCAG
ATGGTAAGCCCTCCCGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATCCTTTTTGATAATCTCA
TGACCAAAATCCCTTAACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTCAGTGAGCGACGAAGCGG
AAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCC
CAGGCTTTACACTTTATGCTTCCGGCTCGTATGTTGTGTGGAATTGTGAGCGGATAACAATTTCACACAG
GAAACAGCTATGACCATGATTACGCCAAGCGCGCAATTAACCCTCACTAAAGGGAACAAAAGCTGGAGCT
GCAAGCTT

TABLE 18
Nucleotide sequence of plasmid pLenti6/V5-D-TOPO ™.

AATGTAGTCTTATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATcGTGCCTTATTAGGAAGGC
AACAGACGGGTCTGACATGGATTGGACGAACCACTGAATTGCCGCATTGCAGAGATATTGTATTTAAGTG
CCTAGCTCGATACATAAACGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCGGTTA
AGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCT
TCAGACAGGATCAGAAGAACTTAGATCATTATATAATACAGTAGCAACCCTCTATTGTGTGCATCAAAGG
ATAGAGATAAAAGACACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTGGAGAAGTGAATTAT
ATAAATATAAAGTAGTAAAAATTGAACCATTAGGAGTAGCACCCACCAAGGCAAAGAGAAGAGTGGTGCA
GAGAGAAAAAAGAGCAGTGGGAATAGGAGCTTTGTTCCTTGGGTTCTTGGGAGCAGCAGGAAGCACTATG
GGCGCAGCGTCAATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTGCTCTGGA
AAACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTGA
AGAATCGCAAAACCAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCATTATCGTT
TCAGACCCACCTCCCAACCCCGAGGGGACCCGACAGGCCCGAAGGAATAGAAGAAGAAGGTGGAGAGAGA
GACAGAGACAGATCCATTCGATTAGTGAACGGATCTCGACGGTATCGATAAGCTTGGGAGTTCCGCGTTA
CATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGTACATCTACGTATT
AGTCATCGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAPAATCAACGGGACT
TTCCAAAATGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTGACCTCCAT
AGAAGACACCGACTCTAGAGGATCCACTAGTCCAGTGTGGTGGAATTGATCCCTTCACCAAGGGCTCGAG
TCTAGAGGGCCCGCGGTTCGAAGGTAAGCCTATCCCTAACCCTCTCCTCGGTCTCGATTCTACGCGTACC
GGTTAGTAATGAGTTTGGAATTAATTCTGTGGAATGTGTGTCAGTTAGGGTGTGGAAAGTCCCCAGGCTC
CCCAGGCAGGCAGAAGTATGCAAAGCATGCATCTCAATTAGTCAGCAACCAGGTGTGGAAAGTCCCCAGG
CTCCCCAGCAGGCAGAAGTATGCAAAGCATGCATCTCAATTAGTCAGCAACCATAGTCCCGCCCCTAACT
CCGCCCATCCCGCCCCTAACTCCGCCCAGTTCCGCCCATTCTCCGCCCCATGGCTGACTAATTTTTTTTA
TTTATGCAGAGGCCGAGGCCGCCTCTGCCTCTGAGCTATTCCAGAAGTAGTGAGGAGGCTTTTTTGGAGG
CCTAGGCTTTTGCAAAAAGCTCCCGGGAGCTTGTATATCCATTTTCGGATCTGATCAGCACGTGTTGACA
ATTAATCATCGGCATAGTATATCGGCATAGTATAATACGACAAGGTGAGGAACTAAACCATGGCCAAGCC
TTTGTCTCAAGAAGAATCCACCCTCATTGAAAGAGCAACGGCTACAATCAACAGCATCCCCATCTCTGAA
GACTACAGCGTCGCCAGCGCAGCTCTCTCTAGCGACGGCCGCATCTTCACTGGTGTCAATGTATATCATT
TTACTGGGGGACCTTGTGCAGAACTCGTGGTGCTGGGCACTGCTGCTGCTGCGGCAGCTGGCAACCTGAC
TTGTATCGTCGCGATCGGAAATGAGAACAGGGGCATCTTGAGCCCCTGCGGACGGTGCCGACAGGTGCTT
CTCGATCTGCATCCTGGGATCAAAGCCATAGTGAAGGACAGTGATGGACAGCCGACGGCAGTTGGGATTC
GTGAATTGCTGCCCTCTGGTTATGTGTGGGAGGGCTAAGCACAATTCGAGCTCGGTACCTTTAAGACCAA
TGACTTACAAGGCAGCTGTAGATCTTAGCCACTTTTTAAAAGAAAAGGGGGGACTGGAAGGGCTAATTCA
CTCCCAACGAAGACAAGATCTGCTTTTTGCTTGTACTGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAG
TGTGTGCCCGTCTGTTGTGTGACTCTGGTAACTAGAGATCCCTCAGACCCTTTTAGTCAGTGTGGAAAAT
CTCTAGCAGTAGTAGTTCATGTCATCTTATTATTCAGTATTTATAACTTGCAAAGAAATGAATATCAGAG
AGTGAGAGGAACTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTATCATGTCTG
GCTCTAGCTATCCCGCCCCTAACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCGGCCTCTGAGCTATTCCAGAA
GTAGTGAGGAGGCTTTTTTGGAGGCCTAGGGACGTACCCAATTCGCCCTATAGTGAGTCGTATTACGCGC
GCTCACTGGCCGTCGTTTTACAACGTCGTGACTGGGAAAACCCTGGCGTTACCCAACTTAATCGCCTTGC
AGCACATCCCCCTTTCGCCAGCTGGCGTAATAGCGAAGAGGCCCGCACCGATCGCCCTTCCCAACAGTTG
CGCAGCCTGAATGGCGAATGGGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCCTTCCTTTCTCGC
CACGTTCGCCGGCTTTCCCCGTCAAGCTCTAAATCGGGGGCTCCCTTTAGGGTTCCGATTTAGTGCTTTA
CGGCACCTCGACCCCAAAAPACTTGATTAGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTGGAACAACACT
CAACCCTATCTCGGTCTATTCTTTTGATTTATAAGGGATTTTGCCGATTTCGGCCTATTGGTTAAAAAAT
GAGCTGATTTAACAAAAATTTAACGCGAATTTTAACAAAATATTAACGCTTACAATTTAGGTGGCACTTT
TCGGGGAAATGTGCGCGGAACCCCTATTTGTTTATTTTTCTAAATACATTCAAATATGTATCCGCTCATG
AGACAATAACCCTGATAAATGCTTCAATAATATTGAAAAAGGAAGAGTATGAGTATTCAACATTTCCGTG
TCGCCCTTATTCCCTTTTTTGCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAATGACTTGGT
TGAGTACTCACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCGCAAACTATTAACTGGC
GAACTACTTACTCTAGCTTCCCGGCAACAATTAATAGACTGGATGGAGGCGGATAAAGTTGCAGGACCAC
TTCTGCGCTCGGCCCTTCCGGCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAGTTATCTACACGACGGGGAGT
CAGGCAACTATGGATGAACGAAATAGACAGATCGCTGAGATAGGTGCCTCACTGATTAAGCATTGGTAAC
TGTCAGACCAAGTTTACTCATATATACTTTAGATTGATTTAAAACTTCATTTTTAATTTAAAAGGATCTA
GGTGAAGATCCTTTTTGATAATCTCATGACCAAAATCCCTTAACGTGAGTTTTCGTTCCACTGAGCGTCA
GACCCCGTAGAAAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTTTTTCCGAAGGT
AACTGGCTTCAGCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGGAAAAACGCCAGCAAC
GCGGCCTTTTTACGGTTCCTGGCCTTTTGCTGGCCTTTTGCTCACATGTTCTTTCCTGCGTTATCCCCTG
ATTCTGTGGATAACCGTATTACCGCCTTTGAGTGAGCTGATACCGCTCGCCGCAGCCGAACGACCGAGCG
CAGCGAGTCAGTGAGCGAGGAAGCGGAAGAGCGCCCAATACGCAAACCGCCTCTCCCCGCGCGTTGGCCG
ATTCATTAATGCAGCTGGCACGACAGGTTTCCCGACTGGAAAGCGGGCAGTGAGCGCAACGCAATTAATG
TGAGTTAGCTCACTCATTAGGCACCCCAGGCTTTACACTTTATGCTTCCGGCTCGTATGTTGTGTGGAAT
TGTGAGCGGATAACAATTTCACACAGGAAACAGCTATGACCATGATTACGCCAAGCGCGCAATTAACCCT
CACTAAAGGGAACAAAAGCTGGAGCTGCAAGCTT

TABLE 19
Nucleotide sequence of pLenti4/V5-DEST.

AATGTAGTCTTATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTTATTAGGAAGGC
AACAGACGGGTCTGACATGGATTGGACGAACCACTGAATTGCCGCATTGCAGAGATATTGTATTTAAGTG
CCTAGCTCGATACATAAACGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCGGTTA
AGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCT
TCAGACAGGATCAGAAGAACTTAGATCATTATATAATACAGTAGCAACCCTCTATTGTGTGCATCAAAGG
ATAGAGATAAAAGACACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTGGAGAAGTGAATTAT
ATAAATATAAAGTAGTAAAAATTGAACCATTAGGAGTAGCACCCACCAAGGCAAAGAGAAGAGTGGTGCA
GAGAGAAAAAAGAGCAGTGGGAATAGGAGCTTTGTTCCTTGGGTTCTTGGGAGCAGCAGGAAGCACTATG
GGCGCAGCGTCAATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCPAGCAGCTCCA
GGCAAGAATCCTGGCTGTGGAAAGATACCTAAAGGATCAACAGCTCCTGGGGATTTGGGGTTGCTCTGGA
AAACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTGA
AGAATCGCAAAACCAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCATTATCGTT
TCAGACCCACCTCCCAACCCCGAGGGGACCCGACAGGCCCGAAGGAATAGAAGAAGAAGGTGGAGAGAGA
GACAGAGACAGATCCATTCGATTAGTGAACGGATCTCGACGGTATCGATAAGCTTGGGAGTTCCGCGTTA
CATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGTACATCTACGTATT
AGTCATCGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTGACCTCCAT
AGAAGACACCGACTCTAGAGGATCCACTAGTCCAGTGTGGTGGAATTCTGCAGATATCAACAAGTTTGTA
CAAAAAAGCTGAACGAGAAACGTAAAATGATATAAATATCAATATATTAAATTAGATTTTGCATAAAAAA
CAGACTACATAATACTGTAAAACACAACATATCCAGTCACTATGGCGGCCGCATTAGGCACCCCAGGCTT
TACACTTTATGCTTCCGGCTCGTATAATGTGTGGATTTTGAGTTAGGATCCGGCGAGATTTTCAGGAGCT
AAGGAAGCTAAAATGGAGAAAAAAATCACTGGATATACCACCGTTGATATATCCCAATGGCATCGTAAAG
AACATTTTGAGGCATTTCAGTCAGTTGCTCAATGTACCTATAACCAGACCGTTCAGCTGGATATTACGGC
CTTTTTAAAGACCGTAAAGAAAAATAAGCACAAGTTTTATCCGGCCTTTATTCACATTCTTGCCCGCCTG
ATGAATGCTCATCCGGAATTCCGTATGGCAATGAAAGACGGTGAGCTGGTGATATGGGATAGTGTTCACC
CTTGTTACACCGTTTTCCATGAGCAAACTGAAACGTTTTCATCGCTCTGGAGTGAATACCACGACGATTT
CCGGCAGTTTCTACACATATATTCGCAAGATGTGGCGTGTTACGGTGAAAACCTGGCCTATTTCCCTAAA
GGGTTTATTGAGAATATGTTTTTCGTCTCAGCCAATCCCTGGGTGAGTTTCACCAGTTTTGATTTAAACG
TGGCCAATATGGACAACTTCTTCGCCCCCGTTTTCACCATGGGCAAATATTATACGCAAGGCGACAAGGT
GCTGATGCCGCTGGCGATTCAGGTTCATCATGCCGTCTGTGATGGCTTCCATGTCGGCAGAATGCTTAAT
GAATTACAACAGTACTGCGATGAGTGGCAGGGCGGGGCGTAAAGATCTGGATCCGGCTTACTAAAAGCCA
GATAACAGTATGCGTATTTGCGCGCTGATTTTTGCGGTATAAGAATATATACTGATATGTATACCCGAAG
TATGTCAAAAAGAGGTGTGCTATGAAGCAGCGTATTACAGTGACAGTTGACAGCGACAGCTATCAGTTGC
TCAAGGCATATATGATGTCAATATCTCCGGTCTGGTAAGCACAACCATGCAGAATGAAGCCCGTCGTCTG
CGTGCCGAACGCTGGAAAGCGGAAAATCAGGAAGGGATGGCTGAGGTCGCCCGGTTTATTGAAATGAACG
GCTCTTTTGCTGACGAGAACAGGGACTGGTGAAATGCAGTTTAAGGTTTACACCTATAAAAGAGAGAGCC
GTTATCGTCTGTTTGTGGATGTACAGAGTGATATTATTGACACGCCCGGGCGACGGATGGTGATCCCCCT
GGCCAGTGCACGTCTGCTGTCAGATAAAGTCTCCCGTGAACTTTACCCGGTGGTGCATATCGGGGATGAA
AGCTGGCGCATGATGACCACCGATATGGCCAGTGTGCCGGTCTCCGTTATCGGGGAAGAAGTGGCTGATC
TCAGCCACCGCGAAAATGACATCAAAAACGCCATTAACCTGATGTTCTGGGGAATATAAATGTCAGGCTC
CGTTATACACAGCCAGTCTGCAGGTCGACCATAGTGACTGGATATGTTGTGTTTTACAGTATTATGTAGT
CTGTTTTTTATGCAAAATCTAATTTAATATATTGATATTTATATCATTTTACGTTTCTCGTTCAGCTTTC
TTGTACAAAGTGGTTGATATCCAGCACAGTGGCGGCCGCTCGAGTCTAGAGGGCCCGCGGTTCGAAGGTA
AGCCTATCCCTAACCCTCTCCTCGGTCTCGATTCTACGCGTACCGGTTAGTAATGAGTTTGGAATTAATT
CTGTGGAATGTGTGTCAGTTAGGGTGTGGAAAGTCCCCAGGCTCCCCAGGCAGGCAGAAGTATGCAAAGC
ATGCATCTCAATTAGTCAGCAACCAGGTGTGGAAAGTCCCCAGGCTCCCCAGCAGGCAGAAGTATGCAAA
GCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCT
GCCTCTGAGCTATTCCAGAAGTAGTGAGGAGGCTTTTTTGGAGGCCTAGGCTTTTGCAAAAAGCTCCCCC
TGTTGACAATTAATCATCGGCATAGTATATCGGCATAGTATAATACGACAAGGTGAGGAACTAAACCATG
GCCAAGTTGACCAGTGCCGTTCCGGTGCTCACCGCGCGCGACGTCGCCGGAGCGGTCGAGTTCTGGACCG
ACCGGCTCGGGTTCTCCCGGGACTTCGTGGAGGACGACTTCGCCGGTGTGGTCCGGGACGACGTGACCCT
GTTCATCAGCGCGGTCCAGGACCAGGTGGTGCCGGACAACACCCTGGCCTGGGTGTGGGTGCGCGGCCTG
GACGAGCTGTACGCCGAGTGGTCGGAGGTCGTGTCCACGAACTTCCGGGACGCCTCCGGGCCGGCCATGA
CCGAGATCGGCGAGCAGCCGTGGGGGCGGGAGTTCGCCCTGCGCGACCCGGCCGGCAACTGCGTGCACTT
CGTGGCCGAGGAGCAGGACTGACACGTGCTACGAGATTTAAATGGTACCTTTAAGACCAATGACTTACAA
GGCAGCTGTAGATCTTAGCCACTTTTTAAAAGAAAAGGGGGGACTGGAAGGGCTAATTCACTCCCAACGA
AGACAAGATCTGCTTTTTGCTTGTACTGGGTCTCTCTGGTTAGACCAGATCTGAGCCTGGGAGCTCTCTG
GCTAACTAGGGAACCCACTGCTTAAGCCTCAATAAAGCTTGCCTTGAGTGCTTCAAGTAGTGTGTGCCCG
TCTGTTGTGTGACTCTGGTAACTAGAGATCCCTCAGACCCTTTTAGTCAGTGTGGAAAATCTCTAGCAGT
AGTAGTTCATGTCATCTTATTATTCAGTATTTATAACTTGCAAAGAAATGAATATCAGAGAGTGAGAGGA
ACTTGTTTATTGCAGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTATCATGTCTGGCTCTAGCTA
TCCCGCCCCTAACTCCGCCCATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGCTATTCCAGAAGTAGTGAGGA
GGCTTTTTTGGAGGCCTAGGGACGTACCCAATTCGCCCTATAGTGAGTCGTATTACGCGCGCTCACTGGC
CGTCGTTTTACAACGTCGTGACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCCCAACAGTTGCGCAGCCTGA
ATGGCGAATGGGACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTTTACGGCACCTCG
ACCCCAAAAAACTTGATTAGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACAACACTCAACCCTATC
TCGGTCTATTCTTTTGATTTATAAGGGATTTTGCCGATTTCGGCCTATTGGTTAAAAAATGAGCTGATTT
AACAAAAATTTAACGCGAATTTTAACAAAATATTAACGCTTACAATTTAGGTGGCACTTTTCGGGGAAAT
GTGCGCGGAACCCCTATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTAACTGGCGAACTACTTA
CTCTAGCTTCCCGGCAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCA
AGTTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAACTGGCTTC
AGCAGAGCGCAGATACCAAATACTGTTCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCTGATTCTGTGGA
TAACCGTATTACCGCCTTTGAGTGAGCTGATACCGCTCGCCGCAGCCGAACGACCGAGCGCAGCGAGTCA
GTGAGCGAGGAAGCGGAAGAGCGCCCAATACGCAAACCGCCTCTCCCCGCGCGTTGGCCGATTCATTAAT
GCAGCTGGCACGACAGGTTTCCCGACTGGAAAGCGGGCAGTGAGCGCAACGCAATTAATGTGAGTTAGCT
CACTCATTAGGCACCCCAGGCTTTACACTTTATGCTTCCGGCTCGTATGTTGTGTGGAATTGTGAGCGGA
TAACAATTTCACACAGGAAACAGCTATGACCATGATTACGCCAAGCGCGCAATTAACCCTCACTAAAGGG
AACAAAAGCTGGAGCTGCAAGCTT

TABLE 20
Nucleotide sequence of pLenti6/UbC/V5-DEST.

AATGTAGTCTTATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTTATTAGGAAGGC
AACAGACGGGTCTGACATGGATTGGACGAACCACTGAATTGCCGCATTGCAGAGATATTGTATTTAAGTG
CCTAGCTCGATACATAAACGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCGGTTA
AGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCT
TCAGACAGGATCAGAAGAACTTAGATCATTATATAATACAGTAGCAACCCTCTATTGTGTGCATCAAAGG
ATAGAGATAAAAGACACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTGGAGAAGTGAATTAT
ATAAATATAAAGTAGTAAAAATTGAACCATTAGGAGTAGCACCCACCAAGGCAAAGAGAAGAGTGGTGCA
GAGAGAAAAAAGAGCAGTGGGAATAGGAGCTTTGTTCCTTGGGTTCTTGGGAGCAGCAGGAAGCACTATG
GGCGCAGCGTCAATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTGCTCTGGA
AAACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTGA
AGAATCGCAAAACCAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCATTATCGTT
TCAGACCCACCTCCCAACCCCGAGGGGACCCGACAGGCCCGAAGGAATAGAAGAAGAAGGTGGAGAGAGA
GACAGAGACAGATCCATTCGATTAGTGAACGGATCTCGACGGTATCGGATCTGGCCTCCGCGCCGGGTTT
TGGCGCCTCCCGCGGGCGCCCCCCTCCTCACGGCGAGCGCTGCCACGTCAGACGAAGGGCGCAGGAGCGT
CCTGATCCTTCCGCCCGGACGCTCAGGACAGCGGCCCGCTGCTCATAAGACTCGGCCTTAGAACCCCAGT
ATCAGCAGAAGGACATTTTAGGACGGGACTTGGGTGACTCTAGGGCACTCGTTTTCTTTCCAGAGAGCGG
AACAGGCGAGGAAAAGTAGTCCCTTCTCGGCGATTCTGCGGAGGGATCTCCGTGGGGCGGTGAACGCCGA
TGATTATATAAGGACGCGCCGGGTGTGGCACAGCTAGTTCCGTCGCAGCCGGGATTTGGGTCGCGGTTCT
TGTTTGTGGATCGCTGTGATCGTCACTTGGTGAGTAGCGGGCTGCTGGGCTGGCCGGGGCTTTCGTGGCC
GCCGGGCCGCTCGGTGGGACGGAAGCGTGTGGAGAGACCGCCAAGGGCTGTAGTCTGGGTCCGCGAGCAA
GGTTGCCCTGAACTGGGGGTTGGGGGGAGCGCAGCAAAATGGCGGCTGTTCCCGAGTCTTGAATGGAAGA
CGCTTGTGAGGCGGGCTGTGAGGTCGTTGAAACAAGGTGGGGGGCATGGTGGGCGGCAAGAACCCAAGGT
CTTGAGGCCTTCGCTAATGCGGGAAAGCTCTTATTCGGGTGAGATGGGCTGGGGCACCATCTGGGGACCC
TGACGTGAAGTTTGTCACTGACTGGAGAACTCGGTTTGTCGTCTGTTGCGGGGGCGGCAGTTATGCGGTG
CCGTTGGGCAGTGCACCCGTACCTTTGGGAGCGCGCGCCCTCGTCGTGTCGTGACGTCACCCGTTCTGTT
GGCTTATAATGCAGGGTGGGGCCACCTGCCGGTAGGTGTGCGGTAGGCTTTTCTCCGTCGCAGGACGCAG
GGTTCGGGCCTAGGGTAGGCTCTCCTGAATCGACAGGCGCCGGACCTCTGGTGAGGGGAGGGATAAGTGA
GGCGTCAGTTTCTTTGGTCGGTTTTATGTACCTATCTTCTTAAGTAGCTGAAGCTCCGGTTTTGAACTAT
GCGCTCGGGGTTGGCGAGTGTGTTTTGTGAAGTTTTTTAGGCACCTTTTGAAATGTAATCATTTGGGTCA
ATATGTAATTTTCAGTGTTAGACTAGTAAATTGTCCGCTAAATTCTGGCCGTTTTTGGCTTTTTTGTTAG
ACGAAGCTTGGTACCGAGCTCGGATCCACTAGTCCAGTGTGGTGGAATTCTGCAGATATCAACAAGTTTG
TACAAAAAAGCTGAACGAGAAACGTAAAATGATATAAATATCAATATATTAAATTAGATTTTGCATAAAA
AACAGACTACATAATACTGTAAAACACAACATATCCAGTCACTATGGCGGCCGCATTAGGCACCCCAGGC
TTTACACTTTATGCTTCCGGCTCGTATAATGTGTGGATTTTGAGTTAGGATCCGGCGAGATTTTCAGGAG
CTAAGGAAGCTAAAATGGAGAAAAAAATCACTGGATATACCACCGTTGATATATCCCAATGGCATCGTAA
AGAACATTTTGAGGCATTTCAGTCAGTTGCTCAATGTACCTATAACCAGACCGTTCAGCTGGATATTACG
GCCTTTTTAAAGACCGTAAAGAAAAATAAGCACAAGTTTTATCCGGCCTTTATTCACATTCTTGCCCGCC
TGATGAATGCTCATCCGGAATTCCGTATGGCAATGAAAGACGGTGAGCTGGTGATATGGGATAGTGTTCA
CCCTTGTTACACCGTTTTCCATGAGCAAACTGAAACGTTTTCATCGCTCTGGAGTGAATACCACGACGAT
TTCCGGCAGTTTCTACACATATATTCGCAAGATGTGGCGTGTTACGGTGAAAACCTGGCCTATTTCCCTA
AAGGGTTTATTGAGAATATGTTTTTCGTCTCAGCCAATCCCTGGGTGAGTTTCACCAGTTTTGATTTAAA
CGTGGCCAATATGGACAACTTCTTCGCCCCCGTTTTCACCATGGGCAAATATTATACGCAAGGCGACAAG
GTGCTGATGCCGCTGGCGATTCAGGTTCATCATGCCGTCTGTGATGGCTTCCATGTCGGCAGAATGCTTA
ATGAATTACAACAGTACTGCGATGAGTGGCAGGGCGGGGCGTAAAGATCTGGATCCGGCTTACTAAAAGC
CAGATAACAGTATGCGTATTTGCGCGCTGATTTTTGCGGTATAAGAATATATACTGATATGTATACCCGA
AGTATGTCAAAAAGAGGTGTGCTATGAAGCAGCGTATTACAGTGACAGTTGACAGCGACAGCTATCAGTT
GCTCAAGGCATATATGATGTCAATATCTCCGGTCTGGTAAGCACAACCATGCAGAATGAAGCCCGTCGTC
TGCGTGCCGAACGCTGGAAAGCGGAAAATCAGGAAGGGATGGCTGAGGTCGCCCGGTTTATTGAAATGAA
CGGCTCTTTTGCTGACGAGAACAGGGACTGGTGAAATGCAGTTTAAGGTTTACACCTATAAAAGAGAGAG
CCGTTATCGTCTGTTTGTGGATGTACAGAGTGATATTATTGACACGCCCGGGCGACGGATGGTGATCCCC
CTGGCCAGTGCACGTCTGCTGTCAGATAAAGTCTCCCGTGAACTTTACCCGGTGGTGCATATCGGGGATG
AAAGCTGGCGCATGATGACCACCGATATGGCCAGTGTGCCGGTCTCCGTTATCGGGGAAGAAGTGGCTGA
TCTCAGCCACCGCGAAAATGACATCAAAAACGCCATTAACCTGATGTTCTGGGGAATATAAATGTCAGGC
TCCGTTATACACAGCCAGTCTGCAGGTCGACCATAGTGACTGGATATGTTGTGTTTTACAGTATTATGTA
GTCTGTTTTTTATGCAAAATCTAATTTAATATATTGATATTTATATCATTTTACGTTTCTCGTTCAGCTT
TCTTGTACAAAGTGGTTGATATCCAGCACAGTGGCGGCCGCTCGAGTCTAGAGGGCCCGCGGTTCGAAGG
TAAGCCTATCCCTAACCCTCTCCTCGGTCTCGATTCTACGCGTACCGGTTAGTAATGAGTTTGGAATTAA
TTCTGTGGAATGTGTGTCAGTTAGGGTGTGGAAAGTCCCCAGGCTCCCCAGGCAGGCAGAAGTATGCAAA
GCATGCATCTCAATTAGTCAGCAACCAGGTGTGGAAAGTCCCCAGGCTCCCCAGCAGGCAGAAGTATGCA
AAGCATGCATCTCAATTAGTCAGCAACCATAGTCCCGCCCCTAACTCCGCCCATCCCGCCCCTAACTCCG
CCCAGTTCCGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAAAAGCTCCC
GGGAGCTTGTATATCCATTTTCGGATCTGATCAGCACGTGTTGACAATTAATCATCGGCATAGTATATCG
GCATAGTATAATACGACAAGGTGAGGAACTAAACCATGGCCAAGCCTTTGTCTCAAGAAGAATCCACCCT
CATTGAAAGAGCAACGGCTACAATCAACAGCATCCCCATCTCTGAAGACTACAGCGTCGCCAGCGCAGCT
CTCTCTAGCGACGGCCGCATCTTCACTGGTGTCAATGTATATCATTTTACTGGGGGACCTTGTGCAGAAC
TCGTGGTGCTGGGCACTGCTGCTGCTGCGGCAGCTGGCAACCTGACTTGTATCGTCGCGATCGGAAATGA
GAACAGGGGCATCTTGAGCCCCTGCGGACGGTGCCGACAGGTGCTTCTCGATCTGCATCCTGGGATCAAA
GCCATAGTGAAGGACAGTGATGGACAGCCGACGGCAGTTGGGATTCGTGAATTGCTGCCCTCTGGTTATG
TGTGGGAGGGCTAAGCACAATTCGAGCTCGGTACCTTTAAGACCAATGACTTACAAGGCAGCTGTAGATC
TTAGCCACTTTTTAAAAGAAAAGGGGGGACTGGAAGGGCTAATTCACTCCCAACGAAGACAAGATCTGCT
TTTTGCTTGTACTGGGTCTCTCTGGTTAGACCAGATCTGAGCCTGGGAGCTCTCTGGCTAACTAGGGAAC
CCACTGCTTAAGCCTCAATAAAGCTTGCCTTGAGTGCTTCAAGTAGTGTGTGCCCGTCTGTTGTGTGACT
CTGGTAACTAGAGATCCCTCAGACCCTTTTAGTCAGTGTGGAAAATCTCTAGCAGTAGTAGTTCATGTCA
TCTTATTATTCAGTATTTATAACTTGCAAAGAAATGAATATCAGAGAGTGAGAGGAACTTGTTTATTGCA
GCTTATAATGGTTACAAATAAAGCAATAGCATCACAAATTTCACAAATAPAGCATTTTTTTCACTGCATT
CTAGTTGTGGTTTGTCCAAACTCATCAATGTATCTTATCATGTCTGGCTCTAGCTATCCCGCCCCTAACT
CCGCCCATCCCGCCCCTAACTCCGCCCAGTTCCGCCCATTCTCCGCCCCATGGCTGACTAATTTTTTTTA
TTTATGCAGAGGCCGAGGCCGCCTCGGCCTCTGAGCTATTCCAGAAGTAGTGAGGAGGCTTTTTTGGAGG
CCTAGGGACGTACCCAATTCGCCCTATAGTGAGTCGTATTACGCGCGCTCACTGGCCGTCGTTTTACAAC
GTCGTGACTGGGAAAACCCTGGCGTTACCCAACTTAATCGCCTTGCAGCACATCCCCCTTTCGCCAGCTG
GCGTAATAGCGAAGAGGCCCGCACCGATCGCCCTTCCCAACAGTTGCGCAGCCTGAATGGCGAATGGGAC
GCGCCCTGTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGACCCCAAAAAACTT
GATTAGGGTGATGGTTCACGTAGTGGGCCATCGCCCTGATAGACGGTTTTTCGCCCTTTGACGTTGGAGT
CCACGTTCTTTAATAGTGGACTCTTGTTCCAAACTGGAACAACACTCAACCCTATCTCGGTCTATTCTTT
TGATTTATAAGGGATTTTGCCGATTTCGGCCTATTGGTTAAAAAATGAGCTGATTTAACAAAAATTTAAC
GCGAATTTTAACAAAATATTAACGCTTACAATTTAGGTGGCACTTTTCGGGGAAATGTGCGCGGAACCCC
TATTTGTTTATTTTTCTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTATTCCCTTTTTTGCGG
CATTTTGCCTTCCTGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGACGCCGGGC
AAGAGCAACTCGGTCGCCGCATACACTATTCTCAGAATGACTTGGTTGAGTACTCACCAGTCACAGAAAA
GCATCTTACGGATGGCATGACAGTAAGAGAATTATGCAGTGCTGCCATAACCATGAGTGATAACACTGCG
GCCAACTTACTTCTGACAACGATCGGAGGACCGAAGGAGCTAACCGCTTTTTTGCACAACATGGGGGATC
ATGTAACTCGCCTTGATCGTTGGGAACCGGAGCTGAATGAAGCCATACCAAACGACGAGCGTGACACCAC
GATGCCTGTAGCAATGGCAACAACGTTGCGCAAACTATTAACTGGCGAACTACTTACTCTAGCTTCCCGG
CAACAATTAATAGACTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTGCAGCACTGGGGCC
AGATGGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCATATA
TACTTTAGATTGATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTTGG
ACTCAAGACGATAGTTACCGGATAAGGCGCAGCGGTCGGGCTGAACGGGGGGTTCGTGCACACAGCCCAG
CTTGGAGCGAACGACCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGATAACCGTATTACCG
CCTTTGAGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAGTTAGCTCACTCATTAGGCAC
CCCAGGCTTTACACTTTATGCTTCCGGCTCGTATGTTGTGTGGAATTGTGAGCGGATAACAATTTCACAC
AGGAAACAGCTATGACCATGATTACGCCAAGCGCGCAATTAACCCTCACTAAAGGGAACAAAAGCTGGAG
CTGCAAGCTT

TABLE 21
Nucleotide sequence of plasmid pLP1.

TTGGCCCATTGCATACGTTGTATCCATATCATAATATGTACATTTATATTGGCTCATGTCCAACATTACC
GCCATGTTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAATGGGCG
TGGATAGCGGTTTGACTCACGGGGATTTCCAAGTCTCCACCCCATTGACGTCAATGGGAGTTTGTTTTGG
CACCAAAATCAACGGCACTTTCCAAAATGTCGTAACAACTCCGCCCCATTGACGCAAATGGGCGGTAGGC
GTGTACGGTGGGAGGTCTATATAAGCAGAGCTCGTTTAGTGAACCGTCAGATCGCCTGGAGACGCCATCC
ACGCTGTTTTGACCTCCATAGAAGACACCGGGACCGATCCAGCCTCCCCTCGAAGCTTACATGTGGTACC
GAGCTCGGATCCTGAGAACTTCAGGGTGAGTCTATGGGACCCTTGATGTTTTCTTTCCCCTTCTTTTCTA
TGGTTAAGTTCATGTCATAGGAAGGGGAGAAGTAACAGGGTACACATATTGACCAAATCAGGGTAATTTT
GCATTTGTAATTTTAAAAAATGCTTTCTTCTTTTAATATACTTTTTTGTTTATCTTATTTCTAATACTTT
CCCTAATCTCTTTCTTTCAGGGCAATAATGATACAATGTATCATGCCTCTTTGCACCATTCTAAAGAATA
ACAGTGATAATTTCTGGGTTAAGGCAATAGCAATATTTCTGCATATAAATATTTCTGCATATAAATTGTA
ACTGATGTAAGAGGTTTCATATTGCTAATAGCAGCTACAATCCAGCTACCATTCTGCTTTTATTTTATGG
TTGGGATAAGGCTGGATTATTCTGAGTCCAAGCTAGGCCCTTTTGCTAATCATGTTCATACCTCTTATCT
TCCTCCCACAGCTCCTGGGCAACGTGCTGGTCTGTGTGCTGGCCCATCACTTTGGCAAAGCACGTGAGAT
CTGAATTCGAGATCTGCCGCCGCCATGGGTGCGAGAGCGTCAGTATTAAGCGGGGGAGAATTAGATCGAT
GGGAAAAAATTCGGTTAAGGCCAGGGGGAAAGAAAAAATATAAATTAAAACATATAGTATGGGCAAGCAG
GGAGCTAGAACGATTCGCAGTTAATCCTGGCCTGTTAGAAACATCAGAAGGCTGTAGACAAATACTGGGA
CAGCTACAACCATCCCTTCAGACAGGATCAGAAGAACTTAGATCATTATATAATACAGTAGCAACCCTCT
ATTGTGTGCATCAAAGGATAGAGATAAAAGACACCAAGGAAGCTTTAGACAAGATAGAGGAAGAGCAAAA
CAAAAGTAAGAAAAAAGCACAGCAAGCAGCAGCTGACACAGGACACAGCAATCAGGTCAGCCAAAATTAC
CCTATAGTGCAGAACATCCAGGGGCAAATGGTACATCAGGCCATATCACCTAGAACTTTAAATGCATGGG
TAAAAGTAGTAGAAGAGAAGGCTTTCAGCCCAGAAGTGATACCCATGTTTTCAGCATTATCAGAAGGAGC
CACCCCACAAGATTTAAACACCATGCTAAACACAGTGGGGGGACATCAAGCAGCCATGCAAATGTTAAAA
GAGACCATCAATGAGGAAGCTGCAGAATGGGATAGAGTGCATCCAGTGCATGCAGGGCCTATTGCACCAG
GCCAGATGAGAGAACCAAGGGGAAGTGACATAGCAGGAACTACTAGTACCCTTCAGGAACAAATAGGATG
GATGACACATAATCCACCTATCCCAGTAGGAGAAATCTATAAAAGATGGATAATCCTGGGATTAAATAAA
ATAGTAAGAATGTATAGCCCTACCAGCATTCTGGACATAAGACAAGGACCAAAGGAACCCTTTAGAGACT
ATGTAGACCGATTCTATAAAACTCTAAGAGCCGAGCAAGCTTCACAAGAGGTAAAAAATTGGATGACAGA
AACCTTGTTGGTCCAAAATGCGAACCCAGATTGTAAGACTATTTTAAAAGCATTGGGACCAGGAGCGACA
CTAGAAGAAATGATGACAGCATGTCAGGGAGTGGGGGGACCCGGCCATAAAGCAAGAGTTTTGGCTGAAG
CAATGAGCCAAGTAACAAATCCAGCTACCATAATGATACAGAAAGGCAATTTTAGGAACCAAAGAAAGAC
TGTTAAGTGTTTCAATTGTGGCAAAGAAGGGCACATAGCCAAAAATTGCAGGGCCCCTAGGAAAAAGGGC
TGTTGGAAATGTGGAAAGGAAGGACACCAAATGAAAGATTGTACTGAGAGACAGGCTAATTTTTTAGGGA
AGATCTGGCCTTCCCACAAGGGAAGGCCAGGGAATTTTCTTCAGAGCAGACCAGAGCCAACAGCCCCACC
AGAAGAGAGCTTCAGGTTTGGGGAAGAGACAACAACTCCCTCTCAGAAGCAGGAGCCGATAGACAAGGAA
CTGTATCCTTTAGCTTCCCTCAGATCACTCTTTGGCAGCGACCCCTCGTCACAATAAAGATAGGGGGGCA
ATTAAAGGAAGCTCTATTAGATACAGGAGCAGATGATACAGTATTAGAAGAAATGAATTTGCCAGGAAGA
TGGAAACCAAAAATGATAGGGGGAATTGGAGGTTTTATCAAAGTAAGACAGTATGATCAGATACTCATAG
AAATCTGCGGACATAAAGCTATAGGTACAGTATTAGTAGGACCTACACCTGTCAACATAATTGGAAGAAA
TCTGTTGACTCAGATTGGCTGCACTTTAAATTTTCCCATTAGTCCTATTGAGACTGTACCAGTAAAATTA
AAGCCAGGAATGGATGGCCCAAAAGTTAAACAATGGCCATTGACAGAAGAAAAAATAAAAGCATTAGTAG
AAATTTGTACAGAAATGGAAAAGGAAGGAAAAATTTCAAAAATTGGGCCTGAAAATCCATACAATACTCC
AGTATTTGCCATAAAGAAAAAAGACAGTACTAAATGGAGAAAATTAGTAGATTTCAGAGAACTTAATAAG
AGAACTCAAGATTTCTGGGAAGTTCAATTAGGAATACCACATCCTGCAGGGTTAAAACAGAAAAAATCAG
TAACAGTACTGGATGTGGGCGATGCATATTTTTCAGTTCCCTTAGATAAAGACTTCAGGAAGTATACTGC
ATTTACCATACCTAGTATAAACAATGAGACACCAGGGATTAGATATCAGTACAATGTGCTTCCACAGGGA
TGGAAAGGATCACCAGCAATATTCCAGTGTAGCATGACAAAAATCTTAGAGCCTTTTAGAAAACAAAATC
CAGACATAGTCATCTATCAATACATGGATGATTTGTATGTAGGATCTGACTTAGAAATAGGGCAGCATAG
AACAAAAATAGAGGAACTGAGACAACATCTGTTGAGGTGGGGATTTACCACACCAGACAAAAAACATCAG
AAAGAACCTCCATTCCTTTGGATGGGTTATGAACTCCATCCTGATAAATGGACAGTACAGCCTATAGTGC
TGCCAGAAAAGGACAGCTGGACTGTCAATGACATACAGAAATTAGTGGGAAAATTGAATTGGGCAAGTCA
GATTTATGCAGGGATTAAAGTAAGGCAATTATGTAAACTTCTTAGGGGAACCAAAGCACTAACAGAAGTA
GTACCACTAACAGAAGAAGCAGAGCTAGAACTGGCAGAAAACAGGGAGATTCTAAAAGAACCGGTACATG
GAGTGTATTATGACCCATCAAAAGACTTAATAGCAGAAATACAGAAGCAGGGGCAAGGCCAATGGACATA
TCAAATTTATCAAGAGCCATTTAAAAATCTGAAAACAGGAAAGTATGCAAGAATGAAGGGTGCCCACACT
AATGATGTGAAACAATTAACAGAGGCAGTACAAAAAATAGCCACAGAAAGCATAGTAATATGGGGAAAGA
CTCCTAAATTTAAATTACCCATACAAAAGGAAACATGGGAAGCATGGTGGACAGAGTATTGGCAAGCCAC
CTGGATTCCTGAGTGGGAGTTTGTCAATACCCCTCCCTTAGTGAAGTTATGGTACCAGTTAGAGAAAGAA
CCCATAATAGGAGCAGAAACTTTCTATGTAGATGGGGCAGCCAATAGGGAAACTAAATTAGGAAAAGCAG
GATATGTAACTGACAGAGGAAGACAAAAAGTTGTCCCCCTAACGGACACAACAAATCAGAAGACTGAGTT
ACAAGCAATTCATCTAGCTTTGCAGGATTCGGGATTAGAAGTAAACATAGTGACAGACTCACAATATGCA
TTGGGAATCATTCAAGCACAACCAGATAAGAGTGAATCAGAGTTAGTCAGTCAAATAATAGAGCAGTTAA
TAAAAAAGGAAAAAGTCTACCTGGCATGGGTACCAGCACACAAAGGAATTGGAGGAAATGAACAAGTAGA
TAAATTGGTCAGTGCTGGAATCAGGAAAGTACTATTTTTAGATGGAATAGATAAGGCCCAAGAAGAACAT
GAGAAATATCACAGTAATTGGAGAGCPATGGCTAGTGATTTTAACCTACCACCTGTAGTAGCAAAAGAAA
TAGTAGCCAGCTGTGATAAATGTCAGCTAAAAGGGGAAGCCATGCATGGACAAGTAGACTGTAGCCCAGG
AATATGGCAGCTAGATTGTACACATTTAGAAGGAAAAGTTATCTTGGTAGCAGTTCATGTAGCCAGTGGA
TATATAGAAGCAGAAGTAATTCCAGCAGAGACAGGGCAAGAAACAGCATACTTCCTCTTAAAATTAGCAG
GAAGATGGCCAGTAAAAACAGTACATACAGACAATGGCAGCAATTTCACCAGTACTACAGTTAAGGCCGC
CTGTTGGTGGGCGGGGATCAAGCAGGAATTTGGCATTCCCTACAATCCCCAAAGTCAAGGAGTAATAGAA
TCTATGAATAAAGAATTAAAGAAAATTATAGGACAGGTAAGAGATCAGGCTGAACATCTTAAGACAGCAG
TACAAATGGCAGTATTCATCCACAATTTTAAAAGAAAAGGGGGGATTGGGGGGTACAGTGCAGGGGAAAG
AATAGTAGACATAATAGCAACAGACATACAAACTAAAGAATTACAAAAACAAATTACAAAAATTCAAAAT
TTTCGGGTTTATTACAGGGACAGCAGAGATCCAGTTTGGAAAGGACCAGCAAAGCTCCTCTGGAAAGGTG
AAGGGGCAGTAGTAATACAAGATAATAGTGACATAAAAGTAGTGCCAAGAAGAAAAGCAAAGATCATCAG
GGATTATGGAAAACAGATGGCAGGTGATGATTGTGTGGCAAGTAGACAGGATGAGGATTAACACATGGAA
TTCCGGAGCGGCCGCAGGAGCTTTGTTCCTTGGGTTCTTGGGAGCAGCAGGAAGCACTATGGGCGCAGCG
TCAATGACGCTGACGGTACAGGCCAGACAATTATTGTCTGGTATAGTGCAGCAGCAGAACAATTTGCTGA
GGGCTATTGAGGCGCAACAGCATCTGTTGCAACTCACAGTCTGGGGCATCAAGCAGCTCCAGGCAAGAAT
CCTGGCTGTGGAAAGATACCTAAAGGATCAACAGCTCCTGGGGATTTGGGGTTGCTCTGGAAAACTCATT
TGCACCACTGCTGTGCCTTGGAATGCTAGTTGGAGTAATAAATCTCTGGAACAGATTTGGAATCACACGA
CCTGGATGGAGTGGGACAGAGAAATTAACAATTACACAAGCTTCCGCGGAATTCACCCCACCAGTGCAGG
CTGCCTATCAGAAAGTGGTGGCTGGTGTGGCTAATGCCCTGGCCCACAAGTATCACTAAGCTCGCTTTCT
TGCTGTCCAATTTCTATTAAAGGTTCCTTTGTTCCCTAAGTCCAACTACTAAACTGGGGGATATTATGAA
GGGCCTTGAGCATCTGGATTCTGCCTAATAAAAAACATTTATTTTCATTGCAATGATGTATTTAAATTAT
TTCTGAATATTTTACTAAAAAGGGAATGTGGGAGGTCAGTGCATTTAAAACATAAAGAAATGAAGAGCTA
GTTCAAACCTTGGGAAAATACACTATATCTTAAACTCCATGAAAGAAGGTGAGGCTGCAAACAGCTAATG
cACATTGGCAACAGCCCCTGATGCCTATGCCTTATTCATCCCTCAGAAAAGGATTCAAGTAGAGGCTTGA
TTTGGAGGTTAAAGTTTTGCTATGCTGTATTTTACATTACTTATTGTTTTAGCTGTCCTCATGAATGTCT
TTTCACTACCCATTTGCTTATCCTGCATCTCTCAGCCTTGACTCCACTCAGTTCTCTTGCTTAGAGATAC
CACCTTTCCCCTGAAGTGTTCCTTCCATGTTTTACGGCGAGATGGTTTCTCCTCGCCTGGCCACTCAGCC
TTAGTTGTCTCTGTTGTCTTATAGAGGTCTACTTGAAGAAGGAAAAACAGGGGGCATGGTTTGACTGTCC
TGTGAGCCCTTCTTCCCTGCCTCCCCCACTCACAGTGACCCGGAATCCCTCGACATGGCAGTCTAGCACT
AGTGCGGCCGCAGATCTGCTTCCTCGCTCACTGACTCGCTGCGCTCGGTCGTTCGGCTGCGGCGAGCGGT
ATCAGCTCACTCAAAGGCGGTAATACGGTTATCCACAGAATCAGGGGATAACGCAGGAAAGAACATGTGA
GCAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGCAACGTTGTTG
CCATTGCTACAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGTTCGA
TGTAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACATATTTGAATGTATTT
AGAAAAATAAACAAATAGGGGTTCCGCGCACATTTCCCCGAAAAGTGCCACCTGACGGGATCCCCTGAGG
GGGCCCCCATGGGCTAGAGGATCCGGCCTCGGCCTCTGCATAAATAAAAAAAATTAGTCAGCCATGAGC

TABLE 22
Nucleotide sequence of plasmid pLP2.

AATGTAGTCTTATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTTATTAGGAAGGC
AACAGACGGGTCTGACATGGATTGGACGAACCACTGAATTCCGCATTGCAGAGATATTGTATTTAAGTGC
CTAGCTCGATACAATAAACGCCATTTGACCATTCACCACATTGGTGTGCACCTCCAAGCTCGAGCTCGTT
TAGTGAACCGTCAGATCGCCTGGAGACGCCATCCACGCTGTTTTGACCTCCATAGAAGACACCGGGACCG
ATCCAGCCTCCCCTCGAAGCTAGTCGATTAGGCATCTCCTATGGCAGGAAGAAGCGGAGACAGCGACGAA
GACCTCCTCAAGGCAGTCAGACTCATCAAGTTTCTCTATCAAAGCAACCCACCTCCCAATCCCGAGGGGA
CCCGACAGGCCCGAAGGAATAGAAGAAGAAGGTGGAGAGAGAGACAGAGACAGATCCATTCGATTAGTGA
ACGGATCCTTAGCACTTATCTGGGACGATCTGCGGAGCCTGTGCCTCTTCAGCTACCACCGCTTGAGAGA
CTTACTCTTGATTGTAACGAGGATTGTGGAACTTCTGGGACGCAGGGGGTGGGAAGCCCTCAAATATTGG
TGGAATCTCCTACAATATTGGAGTCAGGAGCTAAAGAATAGTGCTGTTAGCTTGCTCAATGCCACAGCTA
TAGCAGTAGCTGAGGGGACAGATAGGGTTATAGAAGTAGTACAAGAAGCTTGGCACTGGCCGTCGTTTTA
CAACGTCGTGATCTGAGCCTGGGAGATCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAGATCAGGAAAAC
CCTGGCGTTACCCAACTTAATCGCCTTGCAGCACATCCCCCTTTCGCCAGCTGGCGTAATAGCGAAGAGG
CCCGCACCGATCGCCCTTCCCAACAGTTGCGCAGCCTGAATGGCGAATGGCGCCTGATGCGGTATTTTCT
CCTTACGCATCTGTGCGGTATTTCACACCGCATACGTCAAAGCAACCATAGTACGCGCCCTGTAGCGGCG
CATTAAGCGCGGCGGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTGATTTGGGTGATGGTT
CACGTAGTGGGCCATCGCCCTGATAGACGGTTTTTCGCCCTTTGACGTTGGAGTCCACGTTCTTTAATAG
TGGACTCTTGTTCCAAACTGGAACAACACTCAACCCTATCTCGGGCTATTCTTTTGATTTATAAGGGATT
TTGCCGATTTCGGCCTATTGGTTAAAAAATGAGCTGATTTAACAAAAATTTAACGCGAATTTTAACAAAA
TATTAACGTTTACAATTTTATGGTGCACTCTCAGTACAATCTGCTCTGATGCCGCATAGTTAAGCCAGCC
CCGACACCCGCCAACACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCGAAACGCGCGAGACGAA
AGGGCCTCGTGATACGCCTATTTTTATAGGTTAATGTCATGATAATAATGGTTTCTTAGACGTCAGGTGG
CACTTTTCGGGGAAATGTGCGCGGAACCCCTATTTGTTTATTTTTCTAAATACATTCAAATATGTATCCG
CTCATGAGACAATAACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCGAACTGGATCTCAACAGCGGT
AAGATCCTTGAGAGTTTTCGCCCCGAAGAACGTTTTCCAATGATGAGCACTTTTAAAGTTCTGCTATGTG
GCGCGGTATTATCCCGTATTGACGCCGGGCAAGAGCAACTCGGTCGCCGCATACACTATTCTCAGAATGA
CTTGGTTGAGTACTCACCAGTCACAGAAAAGCATCTTACGGATGGCATGACAGTAAGAGAATTATGCAGT
GCTGCCATAACCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGAGCTGAATGA
AGCCATACCAAACGACGAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGGCGGATAAAGTTGCAG
GACCACTTCTGCGCTCGGCCCTTCCGGCTGGCTGGTTTATTGCTGATAAATCTGGAGCCGGTGAGCGTGG
GTCTCGCGGTATCATTGCAGCACTGGGGCCAGATGGTAAGCCCTCCCGTATCGTAGTTATCTACACGACG
GGGAGTCAGGCAACTATGGATGAACGAAATAGACAGATCGCTGAGATAGGTGCCTCACTGATTAAGCATT
GGTAACTGTCAGACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTACCAACTCTTTTTCC
GAAGGTAACTGGCTTCAGCAGAGCGCAGATACCAAATACTGTTCTTCTAGTGTAGCCGTAGTTAGGCCAC
CACTTCAAGAACTCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGTCGGGTT
TCGCCACCTCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTTCCTGCGTTAT
CCCCTGATTCTGTGGATAACCGTATTACCGCCTTTGAGTGAGCTGATACCGCTCGCCGCAGCCGAACGAC
CGAGCGCAGCGAGTCAGTGAGCGAGGAAGCGGAAGAGCGCCCAATACGCAAACCGCCTCTCCCCGCGCGT
TGGCCGATTCATTAATGCAGCTGGCACGACAGGTTTCCCGACTGGAAAGCGGGCAGTGAGCGCAACGCAA
TTAATGTGAGTTAGCTCACTCATTAGGCACCCCAGGCTTTACACTTTATGCTTCCGGCTCGTATGTTGTG
TGGAATTGTGAGCGGATAACAATTTCACACAGGAAACAGCTATGACATGATTACGAATTCGATGTACGGG
CCAGATATACGCGTATCTGAGGGGACTAGGGTGTGTTTAGGCGAAAAGCGGGGCTTCGGTTGTACGCGGT
TAGGAGTCCCCTCAGGATATAGTAGTTTCGCTTTTGCATAGGGAGGGGGA

TABLE 23
Nucleotide sequence of plasmid pLP/VSVG.

TTGGCCCATTGCATACGTTGTATCCATATCATAATATGTACATTTATATTGGCTCATGTCCAACATTACC
GCCATGTTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAATGGGCG
TGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAACAACTCCGCCCCATTGACGCAAATGGGCGGTAGGC
GTGTACGGTGGGAGGTCTATATAAGCAGAGCTCGTTTAGTGAACCGTCAGATCGCCTGGAGACGCCATCC
ACGCTGTTTTGACCTCCATAGAAGACACCGGGACCGATCCAGCCTCCCCTCGAAGCTTACATGTGGTACC
GAGCTCGGATCCTGAGAACTTCAGGGTGAGTCTATGGGACCCTTGATGTTTTCTTTCCCCTTCTTTTCTA
TGGTTAAGTTCATGTCATAGGAAGGGGAGAAGTAACAGGGTACACATATTGACCAAATCAGGGTAATTTT
GCATTTGTAATTTTAAAAAATGCTTTCTTCTTTTAATATACTTTTTTGTTTATCTTATTTCTAATACTTT
CCCTAATCTCTTTCTTTCAGGGCAATAATGATACAATGTATCATGCCTCTTTGCACCATTCTAAAGAATA
ACAGTGATAATTTCTGGGTTAAGGCAATAGCAATATTTCTGCATATAAATATTTcTGCATATAAATTGTA
ACTGATGTAAGAGGTTTCATATTGCTAATAGCAGCTACAATCCAGCTACCATTCTGCTTTTATTTTATGG
TTGGGATAAGGCTGGATTATTCTGAGTCCAAGCTAGGCCCTTTTGCTAATCATGTTCATACCTCTTATCT
TCCTCCCACAGCTCCTGGGCAACGTGCTGGTCTGTGTGCTGGCCCATCACTTTGGCAAAGCACGTGAGAT
CTGAATTCTGACACTATGAAGTGCCTTTTGTACTTAGCCTTTTTATTCATTGGGGTGAATTGCAAGTTCA
CCATAGTTTTTCCACACAACCAAAAAGGAAACTGGAAAAATGTTCCTTCTAATTACCATTATTGCCCGTC
AAGCTCAGATTTAAATTGGCATAATGACTTAATAGGCACAGCCTTACAAGTCAAAATGCCCAAGAGTCAC
AAGGCTATTCAAGCAGACGGTTGGATGTGTCATGCTTCCAAATGGGTCACTACTTGTGATTTCCGCTGGT
ATGGACCGAAGTATATAACACATTCCATCCGATCCTTCACTCCATCTGTAGAACAATGCAAGGAAAGCAT
TGAACAAACGAAACAAGGAACTTGGCTGAATCCAGGCTTCCCTCCTCAAAGTTGTGGATATGCAACTGTG
ACGGATGCCGAAGCAGTGATTGTCCAGGTGACTCCTCACCATGTGCTGGTTGATGAATACACAGGAGAAT
GGGTTGATTCACAGTTCATCAACGGAAAATGCAGCAATTACATATGCCCCACTGTCCATAACTCTACAAC
CTGGCATTCTGACTATAAGGTCAAAGGGCTATGTGATTCTAACCTCATTTCCATGGACATCACCTTCTTC
TCAGAGGACGGAGAGCTATCATCCCTGGGAAAGGAGGGCACAGGGTTCAGAAGTAACTACTTTGCTTATG
AAACTGGAGGCAAGGCCTGCAAAATGCAATACTGCAAGCATTGGGGAGTCAGACTCCCATCAGGTGTCTG
GTTCGAGATGGCTGATAAGGATCTCTTTGCTGCAGCCAGATTCCCTGAATGCCCAGAAGGGTCAAGTATC
TCTGCTCCATCTCAGACCTCAGTGGATGTAAGTCTAATTCAGGACGTTGAGAGGATCTTGGATTATTCCC
TCTGCCAAGAAACCTGGAGCAAAATCAGAGCGGGTCTTCCAATCTCTCCAGTGGATCTCAGCTATCTTGC
TCCTAAAAACCCAGGAACCGGTCCTGCTTTCACCATAATCAATGGTACCCTAAAATACTTTGAGACCAGA
TACATCAGAGTCGATATTGCTGCTCCAATCCTCTCAAGAATGGTCGGAATGATCAGTGGAACTACCACAG
AAAGGGAACTGTGGGATGACTGGGCACCATATGAAGACGTGGAAATTGGACCCAATGGAGTTCTGAGGAC
CAGTTCAGGATATAAGTTTCCTTTATACATGATTGGACATGGTATGTTGGACTCCGATCTTCATCTTAGC
TCAAAGGCTCAGGTGTTCGAACATCCTCACATTCAAGACGCTGCTTCGCAACTTCCTGATGATGAGAGTT
TATTTTTTGGTGATACTGGGCTATCCAAAPATCCAATCGAGCTTGTAGAAGGTTGGTTCAGTAGTTGGAA
AAGCTCTATTGCCTCTTTTTTCTTTATCATAGGGTTAATCATTGGACTATTCTTGGTTCTCCGAGTTGGT
ATCCATCTTTGCATTAAATTAAAGCACACCAAGAAAAGACAGATTTATACAGACATAGAGATGAACCGAC
TTGGAAAGTAACTCAAATCCTGCACAACAGATTCTTCATGTTTGGACCAAATCAACTTGTGATACCATGC
TCAAAGAGGCCTCAATTATATTTGAGTTTTTAATTTTTATGAAAAAAAAAAAAAAAAACGGAATTCACCC
CACCAGTGCAGGCTGCCTATCAGAAAGTGGTGGCTGGTGTGGCTAATGCCCTGGCCCACAAGTATCACTA
AGCTCGCTTTCTTGCTGTCCAATTTCTATTAAAGGTTCCTTTGTTCCCTAAGTCCAACTACTAAACTGGG
GGATATTATGAAGGGCCTTGAGCATCTGGATTCTGCCTAATAAAAAACATTTATTTTCATTGCAATGATG
TATTTAAATTATTTCTGAATATTTTACTAAAAAGGGAATGTGGGAGGTCAGTGCATTTAAAACATAAAGA
AATGAAGAGCTAGTTCAAACCTTGGGAAAATACACTATATCTTAAACTCCATGAAAGAAGGTGAGGCTGC
AAACAGCTAATGCACATTGGCAACAGCCCCTGATGCCTATGCCTTATTCATCCCTCAGAAAAGGATTCAA
GTAGAGGCTTGATTTGGAGGTTAAAGTTTTGCTATGCTGTATTTTACATTACTTATTGTTTTAGCTGTCC
TCATGAATGTCTTTTCACTACCCATTTGCTTATCCTGCATCTCTCAGCCTTGACTCCACTCAGTTCTCTT
GCTTAGAGATACCACCTTTCCCCTGAAGTGTTCCTTCCATGTTTTACGGCGAGATGGTTTCTCCTCGCCT
GGCCACTCAGCCTTAGTTGTCTCTGTTGTCTTATAGAGGTCTACTTGAAGAAGGAAAAACAGGGGGCATG
GTTTGACTGTCCTGTGAGCCCTTCTTCCCTGCCTCCCCCACTCACAGTGACCCGGAATCCCTCGACATGG
CAGTCTAGCACTAGTGCGGCCGCAGATCTGCTTCCTCGCTCACTGACTCGCTGCGCTCGGTCGTTCGGCT
GCGGCGAGCGGTATCAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATC
AGTGAGGCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCATTCTGAGA
ATAGTGTATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAATA
CTCATACTCTTCCTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCACCTGACGG
GATCCCCTGAGGGGGCCCCCATGGGCTAGAGGATCCGGCCTCGGCCTCTGCATAAATAAAAAAAATTAGT
CAGCCATGAGC

TABLE 28
Nucleotide sequence of plasmid pcDNA ™6.2/V5-DEST.

GACGGATCGGGAGATCTCCCGATCCCCTATGGTGCACTCTCAGTACAATCTGCTCTGATGCCGCATAGTT
AAGCCAGTATCTGCTCCCTGCTTGTGTGTTGGAGGTCGCTGAGTAGTGCGCGAGCAAAATTTAAGCTACA
ACAAGGCAAGGCTTGACCGACAATTGCATGAAGAATCTGCTTAGGGTTAGGCGTTTTGCGCTGCTTCGCG
ATGTACGGGCCAGATATACGCGTTGACATTGATTATTGACTAGTTATTAATAGTAATCAATTACGGGGTC
ATTAGTTCATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGAGCTCTCTGGCTAACTAGAGAACCCA
CTGCTTACTGGCTTATCGAAATTAATACGACTCACTATAGGGAGACCCAAGCTGGCTAGTTAAGCTATCA
ACAAGTTTGTACAAAAAAGCTGAACGAGAAACGTAAAATGATATAAATATCAATATATTAAATTAGATTT
TGCATAAAAAACAGACTACATAATACTGTAAAACACAACATATCCAGTCACTATGATTCAACTACTTAGA
TGGTATTAGTGACCTGTAGTCGACCGACAGCCTTCCAAATGTTCTTCGGGTGATGCTGCCAACTTAGTCG
ACCGACAGCCTTCCAAATGTTCTTCTCAAACGGAATCGTCGTATCCAGCCTACTCGCTATTGTCCTCAAT
GCCGTATTAAATCATAAAAAGAAATAAGAAAAAGAGGTGCGAGCCTCTTTTTTGTGTGACAAAATAAAAA
CATCTACCTATTCATATACGCTAGTGTCATAGTCCTGAAAATCATCTGCATCAAGAACAATTTCACAACT
CTTATACTTTTCTCTTACAAGTCGTTCGGCTTCATCTGGATTTTCAGCCTCTATACTTACTAAACGTGAT
AAAGTTTCTGTAATTTCTACTGTATCGACCTGCAGACTGGCTGTGTATAAGGGAGCCTGACATTTATATT
CCCCAGAACATCAGGTTAATGGCGTTTTTGATGTCATTTTCGCGGTGGCTGAGATCAGCCACTTCTTCCC
CGATAACGGAGACCGGCACACTGGCCATATCGGTGGTCATCATGCGCCAGCTTTCATCCCCGATATGCAC
CACCGGGTAAAGTTCACGGGAGACTTTATCTGACAGCAGACGTGCACTGGCCAGGGGGATCACCATCCGT
CGCCCGGGCGTGTCAATAATATCACTCTGTACATCCACAAACAGACGATAACGGCTCTCTCTTTTATAGG
TGTAAACCTTAAACTGCATTTCACCAGTCCCTGTTCTCGTCAGCAAAAGAGCCGTTCATTTCAATAAACC
GGGCGACCTCAGCCATCCCTTCCTGATTTTCCGCTTTCCAGCGTTCGGCACGCAGACGACGGGCTTCATT
CTGCATGGTTGTGCTTACCAGACCGGAGATATTGACATCATATATGCCTTGAGCAACTGATAGCTGTCGC
TGTCAACTGTCACTGTAATACGCTGCTTCATAGCACACCTCTTTTTGACATACTTCGGGTATACATATCA
GTATATATTCTTATACCGCAAAAATCAGCGCGCAAATACGCATACTGTTATCTGGCTTTTAGTAAGCCGG
ATCCACGCGATTACGCCCCGCCCTGCCACTCATCGCAGTACTGTTGTAATTCATTAAGCATTCTGCCGAC
ATGGAAGCCATCACAGACGGCATGATGAACCTGAATCGCCAGCGGCATCAGCACCTTGTCGCCTTGCGTA
TAATATTTGCCCATGGTGAAAACGGGGGCGAAGAAGTTGTCCATATTGGCCACGTTTAAATCAAAACTGG
TGAAACTCACCCAGGGATTGGCTGAGACGAAAAACATATTCTCAATAAACCCTTTAGGGAAATAGGCCAG
GTTTTCACCGTAACACGCCACATCTTGCGAATATATGTGTAGAAACTGCCGGAAATCGTCGTGGTATTCA
CTCCAGAGCGATGAAAACGTTTCAGTTTGCTCATGGAAAACGGTGTAACAAGGGTGAACACTATCCCATA
TCACCAGCTCACCGTCTTTCATTGCCATACGGAATTCCGGATGAGCATTCATCAGGCGGGCAAGAATGTG
AATAAAGGCCGGATAAAACTTGTGCTTATTTTTCTTTACGGTCTTTAAAAAGGCCGTAATATCCAGCTGA
ACGGTCTGGTTATAGGTACATTGAGCAACTGACTGAAATGCCTCAAAATGTTCTTTACGATGCCATTGGG
ATATATCAACGGTGGTATATCCAGTGATTTTTTTCTCCATTTTAGCTTCCTTAGCTCCTGAAAATCTCGA
TAACTCAAAAAATACGCCCGGTAGTGATCTTATTTCATTATGCTGAAAGTTGGAACCTCTTACGTGCCGA
TCAACGTCTCATTTTCGCCAAAAGTTGGCCCAGGGCTTCCCGGTATCAACAGGGACACCAGGATTTATTT
ATTCTGCGAAGTGATCTTCCGTCACAGGTATTTATTCGGCGCAAAGTGCGTCGGGTGATGCTGCCAACTT
AGTCGACTACAGGTCACTAATACCATCTAAGTAGTTGATTCATAGTGACTGGATATGTTGTGTTTTACAG
TATTATGTAGTCTGTTTTTTATGCAAAATCTAATTTAATATATTGATATTTATATCATTTTACGTTTCTC
GTTCAGCTTTCTTGTACAAAGTGGTTGATCTAGAGGGCCCGCGGTTCGAAGGTAAGCCTATCCCTAACCC
TCTCCTCGGTCTCGATTCTACGCGTACCGGTTAGTAATGAGTTTAAACGGGGGAGGCTAACTGAAACACG
GAAGGAGACAATACCGGAAGGAACCCGCGCTATGACGGCAATAAAAAGACAGAATAAAACGCACGGGTGT
TGGGTCGTTTGTTCATAAACGCGGGGTTCGGTCCCAGGGCTGGCACTCTGTCGATACCCCACCGAGACCC
CATTGGGGCCAATACGCCCGCGTTTCTTCCTTTTCCCCACCCCACCCCCCAAGTTCGGGTGAAGGCCCAG
GGCTCGCAGCCAACGTCGGGGCGGCAGGCCCTGCCATAGCAGATCTGCGCAGCTGGGGCTCTAGGGGGTA
TCCCCACGCGCCCTGTAGCGGCGCATTAAGCGCGGCGGGTGTGGTGGTTACGCGCAGCGTGAcCGCTACA
CTTGCCAGCGCCCTAGCGCCCGCTCCTTTCGCTTTCTTCCCTTCCTTTCTCGCCACGTTCGCCGGCTTTC
CCCGTCAAGCTCTAAATCGGGGCATCCCTTTAGGGTTCCGATTTAGTGCTTTACGGCACCTCGACCCCAA
AAAACTTGATTAGGGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAACCCTATCTCGGTCT
ATTCTTTTGATTTATAAGGGATTTTGGGGATTTCGGCCTATTGGTTAAAAAATGAGCTGATTTAACAAAA
ATTTAACGCGAATTAATTCTGTGGAATGTGTGTCAGTTAGGGTGTGGAAAGTCCCCAGGCTCCCCAGCAG
GCAGAAGTATGCAAAGCATGCATCTCAATTAGTCAGCAACCAGGTGTGGAAAGTCCCCAGGCTCCCCAGC
AGGCAGAAGTATGCAAAGCATGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTTTTATTTATGCAG
AGGCCGAGGCCGCCTCTGCCTCTGAGCTATTCCAGAAGTAGTGAGGAGGCTTTTTTGGAGGCCTAGGCTT
TTGCAAAAAGCTCCCGGGAGCTTGTATATCCATTTTCGGATCTGATCAGCACGTGTTGACAATTAATCAT
CGGCATAGTATATCGGCATAGTATAATACGACAAGGTGAGGAACTAAACCATGGCCAAGCCTTTGTCTCA
AGAAGAATCCACCCTCATTGAAAGAGCAACGGCTACAATCAACAGCATCCCCATCTCTGAAGACTACAGC
GTCGCCAGCGCAGCTCTCTCTAGCGACGGCCGCATCTTCACTGGTGTCAATGTATATCATTTTACTGGGG
GACCTTGTGCAGAACTCGTGGTGCTGGGCACTGCTGCTGCTGCGGCAGCTGGCAACCTGACTTGTATCGT
CGCGATCGGAAATGAGAACAGGGGCATCTTGAGCCCCTGCGGACGGTGCCGACAGGTGCTTCTCGATCTG
CATCCTGGGATCAAAGCCATAGTGAAGGACAGTGATGGACAGCCGACGGCAGTTGGGATTCGTGAATTGC
TGCCCTCTGGTTATGTGTGGGAGGGCTAAGCACTTCGTGGCCGAGGAGCAGGACTGACACGTGCTACGAG
ATTTCGATTCCACCGCCGCCTTCTATGAAAGGTTGGGCTTCGGAATCGTTTTCCGGGACGCCGGCTGGAT
GATCCTCCAGCGCGGGGATCTCATGCTGGAGTTCTTCGCCCACCCCAACTTGTTTATTGCAGCTTATAAT
GGTTACAAATAAAGCAATAGCATCACAAATTTCACAAATAAAGCATTTTTTTCACTGCATTCTAGTTGTG
GTTTGTCCAAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTA
TCCGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGCTA
CACTAGAAGAACAGTATTTGGTATCTGCGCTCTGCTGAAGCCAGTTACCTTCGGAAAAAGAGTTGGTAGC
TCTTGATCCGGCAAACAAACCACCGCTGGTAGCGGTTTTTTTGTTTGCAAGCAGCAGATTACGCGCAGAA
AAAAAGGATCTCAAGAAGATCCTTTGATCTTTTCTACGGGGTCTGACGCTCAGTGGAACGAAAACTCACG
TTAAGGGATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACTTTAAAAG
TGCTCATCATTGGAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTTCTGGGTGAGCA
AAAACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTC

TABLE 29
Nucleotide sequence of plasmid pcDNA ™6.2/GFP-DEST.

GACGGATCGGGAGATCTCCCGATCCCCTATGGTGCACTCTCAGTACAATCTGCTCTGATGCCGCATAGTT
AAGCCAGTATCTGCTCCCTGCTTGTGTGTTGGAGGTCGCTGAGTAGTGCGCGAGCAAAATTTAAGCTACA
ACAAGGCAAGGCTTGACCGACAATTGCATGAAGAATCTGCTTAGGGTTAGGCGTTTTGCGCTGCTTCGCG
ATGTACGGGCCAGATATACGCGTTGACATTGATTATTGACTAGTTATTAATAGTAATCAATTACGGGGTC
ATTAGTTCATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGAGCTCTCTGGCTAACTAGAGAACCCA
CTGCTTACTGGCTTATCGAAATTAATACGACTCACTATAGGGAGACCCAAGCTGGCTAGTTAAGCTATCA
ACAAGTTTGTACAAAAAAGCTGAACGAGAAACGTAAAATGATATAAATATCAATATATTAAATTAGATTT
TGCATAAAAAACAGACTACATAATACTGTAAAACACAACATATCCAGTCACTATGAATCAACTACTTAGA
TGGTATTAGTGACCTGTAGTCGACCGACAGCCTTCCAAATGTTCTTCGGGTGATGCTGCCAACTTAGTCG
ACCGACAGCCTTCCAAATGTTCTTCTCAAACGGAATCGTCGTATCCAGCCTACTCGCTATTGTCCTCAAT
GCCGTATTAAATCATAAAAAGAAATAAGAAAAAGAGGTGCGAGCCTCTTTTTTGTGTGACAAAATAAAAA
CATCTACCTATTCATATACGCTAGTGTCATAGTCCTGAAAATCATCTGCATCAAGAACAATTTCACAACT
CTTATACTTTTCTCTTACAAGTCGTTCGGCTTCATCTGGATTTTCAGCCTCTATACTTACTAAACGTGAT
AAAGTTTCTGTAATTTCTACTGTATCGACCTGCAGACTGGCTGTGTATAAGGGAGCCTGACATTTATATT
CCCCAGAACATCAGGTTAATGGCGTTTTTGATGTCATTTTCGCGGTGGCTGAGATCAGCCACTTCTTCCC
CGATAACGGAGACCGGCACACTGGCCATATCGGTGGTCATCATGCGCCAGCTTTCATCCCCGATATGCAC
CACCGGGTAAAGTTCACGGGAGACTTTATCTGACAGCAGACGTGCACTGGCCAGGGGGATCACCATCCGT
CGCCCGGGCGTGTCAATAATATCACTCTGTACATCCACAAACAGACGATAACGGCTCTCTCTTTTATAGG
TGTAAACCTTAAACTGCATTTCACCAGTCCCTGTTCTCGTCAGCAAAAGAGCCGTTCATTTCAATAAACC
GGGCGACCTCAGCCATCCCTTCCTGATTTTCCGCTTTCCAGCGTTCGGCACGCAGACGACGGGCTTCATT
CTGCATGGTTGTGCTTACCAGACCGGAGATATTGACATCATATATGCCTTGAGCAACTGATAGCTGTCGC
TGTCAACTGTCACTGTAATACGCTGCTTCATAGCACACCTCTTTTTGACATACTTCGGGTATACATATCA
GTATATATTCTTATACCGCAAAAATCAGCGCGCAAATACGCATACTGTTATCTGGCTTTTAGTAGGCCGG
ATCCACGCGATTACGCCCCGCCCTGCCACTCATCGCAGTACTGTTGTAATTCATTAAGCATTCTGCCGAC
ATGGAAGCCATCACAGACGGCATGATGAACCTGAATCGCCAGCGGCATCAGCACCTTGTCGCCTTGCGTA
TAATATTTGCCCATGGTGAAAACGGGGGCGAAGAAGTTGTCCATATTGGCCACGTTTAAATCAAAACTGG
TGAAACTCACCCAGGGATTGGCTGAGACGAAAAACATATTCTCAATAAACCCTTTAGGGAAATAGGCCAG
GTTTTCACCGTAACACGCCACATCTTGCGAATATATGTGTAGAAACTGCCGGAAATCGTCGTGGTATTCA
CTCCAGAGCGATGAAAACGTTTCAGTTTGCTCATGGAAAACGGTGTAACAAGGGTGAACACTATCCCATA
TCACCAGCTCACCGTCTTTCATTGCCATACGGAATTCCGGATGAGCATTCATCAGGCGGGCAAGAATGTG
AATAAAGGCCGGATAAAACTTGTGCTTATTTTTCTTTACGGTCTTTAAAAAGGCCGTAATATCCAGCTGA
ACGGTCTGGTTATAGGTACATTGAGCAACTGACTGAAATGCCTCAAAATGTTCTTTACGATGCCATTGGG
ATATATCAACGGTGGTATATCCAGTGATTTTTTTCTCCATTTTAGCTTCCTTAGCTCCTGAAAATCTCGA
TAACTCAAAAAATACGCCCGGTAGTGATCTTATTTCATTATGGTGAAAGTTGGAACCTCTTACGTGCCGA
TCAACGTCTCATTTTCGCCAAAAGTTGGCCCAGGGCTTCCCGGTATCAACAGGGACACCAGGATTTATTT
ATTCTGCGAAGTGATCTTCCGTCACAGGTATTTATTCGGCGCAAAGTGCGTCGGGTGATGCTGCCAACTT
AGTCGACTACAGGTCACTAATACCATCTAAGTAGTTGATTCATAGTGACTGGATATGTTGTGTTTTACAG
TATTATGTAGTCTGTTTTTTATGCAAAATCTAATTTAATATATTGATATTTATATCATTTTACGTTTCTC
GTTCAGCTTTCTTGTACAAAGTGGTTGATCTAGAGGGCCCCGCGGCTAGCAAAGGAGAAGAACTTTTCAC
TGGAGTTGTCCCAATTCTTGTTGAATTAGATGGTGATGTTAATGGGCACAAATTTTCTGTCAGTGGAGAG
GGTGAAGGTGATGCTACATACGGAAAGCTTACCCTTAAATTTATTTGCACTACTGGAAAACTACCTGTTC
CATGGCCAACACTTGTCACTACTTTCTCTTATGGTGTTCAATGCTTTTCCCGTTATCCGGATCATATGAA
ACGGCATGACTTTTTCAAGAGTGCCATGCCCGAAGGTTATGTACAGGAACGCACTATATCTTTCAAAGAT
GACGGGAACTACAAGACGCGTGCTGAAGTCAAGTTTGAAGGTGATACCCTTGTTAATCGTATCGAGTTAA
AAGGTATTGATTTTAAAGAAGATGGAAACATTCTCGGACACAAACTCGAGTACAACTATAACTCACACAA
TGTATACATCACGGCAGACAAACAAAAGAATGGAATCAAAGCTAACTTCAAAATTCGTCACAACATTGAA
GATGGATCCGTTCAACTAGCAGACCATTATCAACAAAATACTCCAATTGGCGATGGCCCTGTCCTTTTAC
CAGACAACCATTACCTGTCGACACAATCTGCCCTTTCGAAAGATCCCAACGAAAAGCGTGACCACATGGT
CCTTCTTGAGTTTGTAACTGCTGCTGGGATTACACATGGCATGGATGAATAGTAATGAGTCCACGTTTAA
ACGGGGGAGGCTAACTGAAACACGGAAGGAGACAATACCGGAAGGAACCCGCGCTATGACGGCAATAAAA
AGACAGAATAAAACGCACGGGTGTTGGGTCGTTTGTTCATAAACGCGGGGTTCGGTCCCAGGGCTGGCAC
TCTGTCGATACCCCACCGAGACCCCATTGGGGCCAATACGCCCGCGTTTCTTCCTTTTCCCCACCCCACC
CCCCAAGTTCGGGTGAAGGCCCAGGGCTCGCAGCCAACGTCGGGGCGGCAGGCCCTGCCATAGCAGATCT
GCGCAGCTGGGGCTCTAGGGGGTATCCCCACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGCATCCCTTTAGGGTTCCGATTTAG
TGCTTTACGGCACCTCGACCCCAAAAAACTTGATTAGGGTGATGGTTCACGTAGTGGGCCATCGCCCTGA
TAGACGGTTTTTCGCCCTTTGACGTTGGAGTCCACGTTCTTTAATAGTGGACTCTTGTTCCAAACTGGAA
CAACACTCAACCCTATCTCGGTCTATTCTTTTGATTTATAAGGGATTTTGGGGATTTCGGCCTATTGGTT
AAAAAATGAGCTGATTTAACAAAAATTTAACGCGAATTAATTCTGTGGAATGTGTGTCAGTTAGGGTGTG
GAAAGTCCCCAGGCTCCCCAGCAGGCAGAAGTATGCAAAGCATGCATCTCAATTAGTCAGCAACCAGGTG
TGGAAAGTCCCCAGGCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCTCCGCCCCATGGCT
GACTAATTTTTTTTATTTATGCAGAGGCCGAGGCCGCCTCTGCCTCTGAGCTATTCCAGAAGTAGTGAGG
AGGCTTTTTTGGAGGCCTAGGCTTTTGCAAAAAGCTCCCGGGAGCTTGTATATCCATTTTCGGATCTGAT
CAGCACGTGTTGACAATTAATCATCGGCATAGTATATCGGCATAGTATAATACGACAAGGTGAGGAACTA
AACCATGGCCAAGCCTTTGTCTCAAGAAGAATCCACCCTCATTGAAAGAGCAACGGCTACAATCAACAGC
ATCCCCATCTCTGAAGACTACAGCGTCGCCAGCGCAGCTCTCTCTAGCGACGGCCGCATCTTCACTGGTG
TCAATGTATATCATTTTACTGGGGGACCTTGTGCAGAACTCGTGGTGCTGGGCACTGCTGCTGCTGCGGC
AGCTGGCAACCTGACTTGTATCGTCGCGATCGGAAATGAGAACAGGGGCATCTTGAGCCCCTGCGGACGG
TGCCGACAGGTGCTTCTCGATCTGCATCCTGGGATCAAAGCCATAGTGAAGGACAGTGATGGACAGCCGA
CGGCAGTTGGGATTCGTGAATTGCTGCCCTCTGGTTATGTGTGGGAGGGCTAAGCACTTCGTGGCCGAGG
AGCAGGACTGACACGTGCTACGAGATTTCGATTCCACCGCCGCCTTCTATGAAAGGTTGGGCTTCGGAAT
CGTTTTCCGGGACGCCGGCTGGATGATCCTCCAGCGCGGGGATCTCATGCTGGAGTTCTTCGCCCACCCC
AACTTGTTTATTGCAGCTTATAATGGTTACAAATAAAGCAATAGCATCACAAATTTCACAAATAAAGCAT
TTTTTTCACTGCATTCTAGTTGTGGTTTGTCCAAACTCATCAATGTATCTTATCATGTCTGTATACCGTC
GACCTCTAGCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGCTACAGAGTTCTT
GAAGTGGTGGCCTAACTACGGCTACACTAGAAGAACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTTTTTTTGTTT
GCAAGCAGCAGATTACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTGCAATGATACCGCGA
GACCCACGCTCACCGGCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATACCG
CGCCACATAGCAGAACTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCTTCAGCATCTTTTACT
TTCACCAGCGTTTCTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCACATTTCCCCGAAAA
GTGCCACCTGACGTC

TABLE 30
Amino acid sequence of a polypeptide having
β-lactamase activity.

```
Met Gly His Pro Glu Thr Leu Val Lys Val Lys Asp Ala Glu Asp Gln
    1 5 10 15
Leu Gly Ala Arg Val Gly Tyr Ile Glu Leu Asp Leu Asn Ser Gly Lys
                          20 25 30
Ile Leu Glu Ser Phe Arg Pro Glu Glu Arg Phe Pro Met Met Ser Thr
                  35 40 45
Phe Lys Val Leu Leu Cys Gly Ala Val Leu Ser Arg Asp Asp Ala Gly
          50 55 60
Gln Glu Gln Leu Gly Arg Arg Ile His Tyr Ser Gln Asn Asp Leu Val
  65 70 75 80
Glu Tyr Ser Pro Val Thr Glu Lys His Leu Thr Asp Gly Met Thr Val
                                  85 90 95
Arg Glu Leu Cys Ser Ala Ala Ile Thr Met Ser Asp Asn Thr Ala Ala
                        100 105 110
Asn Leu Leu Leu Thr Thr Ile Gly Gly Pro Lys Glu Leu Thr Ala Phe
                115 120 125
Leu His Asn Met Gly Asp His Val Thr Arg Leu Asp His Trp Glu Pro
        130 135 140
Glu Leu Asn Glu Ala Ile Pro Asn Asp Glu Arg Asp Thr Thr Met Pro
145 150 155 160
Val Ala Met Ala Thr Thr Leu Arg Lys Leu Leu Thr Gly Glu Leu Leu
                                165 170 175
Thr Leu Ala Ser Arg Gln Gln Leu Ile Asp Trp Met Glu Ala Asp Lys
                        180 185 190
Val Ala Gly Pro Leu Leu Arg Ser Ala Leu Pro Ala Gly Trp Phe Ile
                195 200 205
Ala Asp Lys Ser Gly Ala Gly Glu Arg Gly Ser Arg Gly Ile Ile Ala
        210 215 220
Ala Leu Gly Pro Asp Gly Lys Pro Ser Arg Ile Val Val Ile Tyr Thr
225 230 235 240
Thr Gly Ser Gln Ala Thr Met Asp Glu Arg Asn Arg Gln Ile Ala Glu
                                245 250 255
Ile Gly Ala Ser Leu Ile Lys His Trp
        260 265
```

TABLE 31
Nucleotide sequence of pLenti4TO/V5-DEST.

aatgtagtcttatgcaatactcttgtagtcttgcaacatggtaacgatgagttagcaacatgccttacaaggaga
gaaaaagcaccgtgcatgccgattggtggaagtaaggtggtacgatcgtgccttattaggaaggcaacagacggg
tctgacatggattggacgaaccactgaattgccgcattgcagagatattgtatttaagtgcctagctcgatacat
aaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaagggaaacca
gaggagctctctcgacgcaggactcggcttgctgaagcgcgcacggcaagaggcgaggggcggcgactggtgagt
acgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgg
gcaagcagggagctagaacgattcgcagttaatcctggcctgttagaaacatcagaaggctgtagacaaatactg
ggacagctacaaccatcccttcagacaggatcagaagaacttagatcattatataatacagtagcaaccctctat
tgtgtgcatcaaaggatagagataaaagacaccaaggaagctttagacaagatagaggaagagcaaaacaaaagt
aagaccaccgcacagcaagcggccgctgatcttcagacctggaggaggagatatgagggacaattggagaagtga
attatataaatataaagtagtaaaaattgaaccattaggagtagcacccaccaaggcaaagagaagagtggtgca
gagagaaaaaagagcagtgggaataggagctttgttccttgggttcttgggagcagcaggaagcactatgggcgc
agcgtcaatgacgctgacggtacaggccagacaattattgtctggtatagtgcagcagcagaacaatttgctgag
ggctattgaggcgcaacagcatctgttgcaactcacagtctggggcatcaagcagctccaggcaagaatcctggc
tgtggaaagatacctaaaggatcaacagctcctggggatttggggttgctctggaaaactcatttgcaccactgc
tgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatga
acaagaattattggaattagataaatgggcaagtttgtggaattggtttaacataacaaattggctgtggtatat
aaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaagg
aatagaagaagaaggtggagagagagacagagacagatccattcgattagtgaacggatctcgacggtatcgata
agcttgggagttccgcgttacataacttacggtaaatggcccgcctggctgaccgcccaacgacccccgcccatt
gacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacgggg
atttccaagtctccaccccattgacgtcaatgggagtttgttttggcaccaaaatcaacgggactttccaaaatg
tcgtaacaactccgccccattgacgcaaatgggcggtaggcgtgtacggtgggaggtctatataagcagagctct
ccctatcagtgatagagatctccctatcagtgatagagatcgtcgactagtccagtgtggtggaattctgcagat
atcaacaagtttgtacaaaaaagctgaacgagaaacgtaaaatgatataaatatcaatatattaaattagatttt
gcataaaaaacagactacataatactgtaaaacacaacatatccagtcactatggcggccgcattaggcacccca
ggctttacactttatgcttccggctcgtataatgtgtggattttgagttaggatccggcgagattttcaggagct
aaggaagctaaaatggagaaaaaaatcactggatataccaccgttgatatatcccaatggcatcgtaaagaacat
tttgaggcatttcagtcagttgctcaatgtacctataaccagaccgttcagctggatattacggcctttttaaag
accgtaaagaaaaataagcacaagttttatccggcctttattcacattcttgcccgcctgatgaatgctcatccg
gaattccgtatggcaatgaaagacggtgagctggtgatatgggatagtgttcacccttgttacaccgttttccat
gagcaaactgaaacgttttcatcgctctggagtgaataccacgacgatttccggcagtttctacacatatattcg
caagatgtggcgtgttacggtgaaaacctggcctatttccctaaagggtttattgagaatatgtttttcgtctca
gccaatccctgggtgagtttcaccagttttgatttaaacgtggccaatatggacaacttcttcgcccccgttttc
accatgggcaaatattatacgcaaggcgacaaggtgctgatgccgctggcgattcaggttcatcatgccgtctgt
gatggcttccatgtcggcagaatgcttaatgaattacaacagtactgcgatgagtggcagggcggggcgtaaaga
tctggatccggcttactaaaagccagataacagtatgcgtatttgcgcgctgatttttgcggtataagaatatat
actgatatgtatacccgaagtatgtcaaaaagaggtgtgctatgaagcagcgtattacagtgacagttgacagcg
acagctatcagttgctcaaggcatatatgatgtcaatatctccggtctggtaagcacaaccatgcagaatgaagc
ccgtcgtctgcgtgccgaacgctggaaagcggaaaatcaggaagggatggctgaggtcgcccggtttattgaaat
gaacggctcttttgctgacgagaacagggactggtgaaatgcagtttaaggtttacacctataaaagagagagcc
gttatcgtctgtttgtggatgtacagagtgatattattgacacgcccgggcgacggatggtgatccccctggcca
gtgcacgtctgctgtcagataaagtctcccgtgaactttacccggtggtgcatatcggggatgaaagctggcyca
tgatgaccaccgatatggccagtgtgccggtctccgttatcggggaagaagtggctgatctcagccaccgcgaaa
atgacatcaaaaacgccattaacctgatgttctggggaatataaatgtcaggctccgttatacacagccagtctg
caggtcgaccatagtgactggatatgttgtgttttacagtattatgtagtctgttttttatgcaaaatctaattt
aatatattgatatttatatcattttacgtttctcgttcagctttcttgtacaaagtggttgatatccagcacagt
ggcggccgctcgagtctagagggcccgcggttcgaaggtaagcctatccctaaccctctcctcggtctcgattct
acgcgtaccggttagtaatgagtttggaattaattctgtggaatgtgtgtcagttagggtgtggaaagtccccag
gctccccaggcaggcagaagtatgcaaagcatgcatctcaattagtcagcaaccaggtgtggaaagtccccaggc
tccccagcaggcagaagtatgcaaagcatgcatctcaattagtcagcaaccatagtcccgcccctaactccgccc
atcccgcccctaactccgcccagttccgcccattctccgccccatggctgactaattttttttatttatgcagag
gccgaggccgcctctgcctctgagctattccagaagtagtgaggaggcttttttggaggcctaggcttttgcaaa
aagctccccctgttgacaattaatcatcggcatagtatatcggcatagtataatacgacaaggtgaggaactaaa
ccatggccaagttgaccagtgccgttccggtgctcaccgcgcgcgacgtcgccggagcggtcgagttctggaccg
accggctcgggttctcccgggacttcgtggaggacgacttcgccggtgtggtccgggacgacgtgaccctgttca
tcagcgcggtccaggaccaggtggtgccggacaacaccctggcctgggtgtgggtgcgcggcctggacgagctgt
acgccgagtggtcggaggtcgtgtccacgaacttccgggacgcctccgggccggccatgaccgagatcggcgagc
agccgtgggggcgggagttcgccctgcgcgacccggccggcaactgcgtgcacttcgtggccgaggagcaggact
gacacgtgctacgagatttaaatggtacctttaagaccaatgacttacaaggcagctgtagatcttagccacttt
ttaaaagaaaaggggggactggaagggctaattcactcccaacgaagacaagatctgctttttgcttgtactggg
tctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagaccc
ttttagtcagtgtggaaaatctctagcagtagtagttcatgtcatcttattattcagtatttataacttgcaaag
aaatgaatatcagagagtgagaggaacttgtttattgcagcttataatggttacaaataaagcaatagcatcaca
aatttcacaaataaagcatttttttcactgcattctagttgtggtttgtccaaactcatcaatgtatcttatcat
gtctggctctagctatcccgcccctaactccgcccatcccgcccctaactccgcccagttccgcccattctccgc
cccatggctgactaattttttttatttatgcagaggccgaggccgcctcggcctctgagctattccagaagtagt
gaggaggcttttttggaggcctagggacgtacccaattcgccctatagtgagtcgtattacgcgcgctcactggc
cgtcgttttacaacgtcgtgactgggaaaaccctggcgttacccaacttaatcgccttgcagcacatcccccttt
cgccagctggcgtaatagcgaagaggcccgcaccgatcgcccttcccaacagttgcgcagcctgaatggcgaatg
ggacgcgccctgtagcggcgcattaagcgcggcgggtgtggtggttacgcgcagcgtgaccgctacacttgccag
cgccctagcgcccgctcctttcgctttcttcccttcctttctcgccacgttcgccggctttccccgtcaagctct
aaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatag
tggactcttgttccaaactggaacaacactcaaccctatctcggtctattcttttgatttataagggattttgcc
gatttcggcctattggttaaaaaatgagctgatttaacaaaaatttaacgcgaattttaacaaaatattaacgct
tacaatttaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaa
catttccgtgtcgcccttattcccttttttgcggcattttgccttcctgtttttgctcacccagaaacgctggtg
aaagtaaaagatgctgaagatcagttgggtgcacgagtgggttacatcgaactggatctcaacagcggtaagatc
cttgagagttttcgccccgaagaacgttttccaatgatgagcacttttaaagttctgctatgtggcgcggtatta
tcccgtattgacgccgggcaagagcaactcggtcgccgcatacactattctcagaatgacttggttgagtactca
ccagtcacagaaaagcatcttacggatggcatgacagtaagagaattatgcagtgctgccataaccatgagtgat
aacactgcggccaacttacttctgacaacgatcggaggaccgaaggagctaaccgcttttttgcacaacatgggg
gatcatgtaactcgccttgatcgttgggaaccggagctgaatgaagccataccaaacgacgagcgtgacaccacg
atgcctgtagcaatggcaacaacgttgcgcaaactattaactggcgaactacttactctagcttcccggcaacaa
ttaatagactggatggaggcggataaagttgcaggaccacttctgcgctcggcccttccggctggctggtttatt
gctgataaatctggagccggtgagcgtgggtctcgcggtatcattgcagcactggggccagatggtaagccctcc
cgtatcgtagttatctacacgacggggagtcaggcaactatggatgaacgaaatagacagatcgctgagataggt
gcctcactgattaagcattggtaactgtcagaccaagtttactcatatatactttagattgatttaaaacttcat
ttttaatttaaaaggatctaggtgaagatcctttttgataatctcatgaccaaaatcccttaacgtgagttttcg
ttccactgagcgtcagaccccgtagaaaagatcaaaggatcttcttgagatcctttttttctgcgcgtaatctgc
tgcttgcaaacaaaaaaaccaccgctaccagcggtggtttgtttgccggatcaagagctaccaactctttttccg
aaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataag
tcgtgtcttaccgggttggactcaagacgatagttaccggataaggcgcagcggtcgggctgaacggggggttcg
tgcacacagcccagcttggagcgaacgacctacaccgaactgagatacctacagcgtgagctatgagaaagcgcc
acgcttcccgaagggagaaaggcggacaggtatccggtaagcggcagggtcggaacaggagagcgcacgagggag
cttccagggggaaacgcctggtatctttatagtcctgtcgggtttcgccacctctgacttgagcgtcgatttttg
tgatgctcgtcaggggggcggagcctatggaaaaacgccagcaacgcggcctttttacggttcctggccttttgc
tggccttttgctcacatgttctttcctgcgttatcccctgattctgtggataaccgtattaccgcctttgagtga
gctgataccgctcgccgcagccgaacgaccgagcgcagcgagtcagtgagcgaggaagcggaagagcgcccaata
cgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaag
cgcgcaattaaccctcactaaagggaacaaaagctggagctgcaagctt

TABLE 32
Nucleotide sequence of pLenti6/TR.

aatgtagtcttatgcaatactcttgtagtcttgcaacatggtaacgatgagttagcaacatgccttacaaggaga
gaaaaagcaccgtgcatgccgattggtggaagtaaggtggtacgatcgtgccttattaggaaggcaacagacggg
tctgacatggattggacgaaccactgaattgccgcattgcagagatattgtatttaagtgcctagctcgatacat
aaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaagggaaacca
gaggagctctctcgacgcaggactcggcttgctgaagcgcgcacggcaagaggcgaggggcggcgactggtgagt
acgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgg
gcaagcagggagctagaacgattcgcagttaatcctggcctgttagaaacatcagaaggctgtagacaaatactg
ggacagctacaaccatcccttcagacaggatcagaagaacttagatcattatataatacagtagcaaccctctat
tgtgtgcatcaaaggatagagataaaagacaccaaggaagctttagacaagatagaggaagagcaaaacaaaagt
aagaccaccgcacagcaagcggccgctgatcttcagacctggaggaggagatatgagggacaattggagaagtga
attatataaatataaagtagtaaaaattgaaccattaggagtagcacccaccaaggcaaagagaagagtggtgca
gagagaaaaaagagcagtgggaataggagctttgttccttgggttcttgggagcagcaggaagcactatgggcgc
agcgtcaatgacgctgacggtacaggccagacaattattgtctggtatagtgcagcagcagaacaatttgctgag
ggctattgaggcgcaacagcatctgttgcaactcacagtctggggcatcaagcagctccaggcaagaatcctggc
tgtggaaagatacctaaaggatcaacagctcctggggatttggggttgctctggaaaactcatttgcaccactgc
tgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatga
acaagaattattggaattagataaatgggcaagtttgtggaattggtttaacataacaaattggctgtggtatat
aaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaagg
aatagaagaagaaggtggagagagagacagagacagatccattcgattagtgaacggatctcgacggtatcgata
agcttgggagttccgcgttacataacttacggtaaatggcccgcctggctgaccgcccaacgacccccgcccatt
gacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacgggg
atttccaagtctccaccccattgacgtcaatgggagtttgttttggcaccaaaatcaacgggactttccaaaatg
tcgtaacaactccgccccattgacgcaaatgggcggtaggcgtgtacggtgggaggtctatataagcagagctcg
tttagtgaaccgtcagatcgcctggagacgccatccacgctgttttgacctccatagaagacaccgactctagag
gatccactagtccagtgtggtggaattctgcagatagcttggtacccggggatcctctagggcctctgagctatt
ccagaagtagtgaagaggcttttttggaggcctaggcttttgcaaaaagctccggatcgatcctgagaacttcag
ggtgagtttggggacccttgattgttctttctttttcgctattgtaaaattcatgttatatggagggggcaaagt
tttcagggtgttgtttagaatgggaagatgtcccttgtatcaccatggaccctcatgataattttgtttctttca
ctttctactctgttgacaaccattgtctcctcttattttcttttcattttctgtaactttttcgttaaactttag
cttgcatttgtaacgaatttttaaattcacttttgtttatttgtcagattgtaagtactttctctaatcactttt
ttttcaaggcaatcagggtatattatattgtacttcagcacagttttagagaacaattgttataattaaatgata
aggtagaatatttctgcatataaattctggctggcgtggaaatattcttattggtagaaacaactacatcctggt
catcatcctgcctttctctttatggttacaatgatatacactgtttgagatgaggataaaatactctgagtccaa
accgggcccctctgctaaccatgttcatgccttcttctttttcctacagctcctgggcaacgtgctggttattgt
gctgtctcatcattttggcaaagaattgtaatacgactcactatagggcgaattgatatgtctagattagataaa
agtaaagtgattaacagcgcattagagctgcttaatgaggtcggaatcgaaggtttaacaacccgtaaactcgcc
cagaagctaggtgtagagcagcctacattgtattggcatgtaaaaaataagcgggctttgctcgacgccttagcc
attgagatgttagataggcaccatactcacttttgccctttagaaggggaaagctggcaagattttttacgtaat
aacgctaaaagttttagatgtgctttactaagtcatcgcgatggagcaaaagtacatttaggtacacggcctaca
gaaaaacagtatgaaactctcgaaaatcaattagcctttttatgccaacaaggtttttcactagagaatgcatta
tatgcactcagcgctgtggggcattttactttaggttgcgtattggaagatcaagagcatcaagtcgctaaagaa
gaaagggaaacacctactactgatagtatgccgccattattacgacaagctatcgaattatttgatcaccaaggt
gcagagccagccttcttattcggccttgaattgatcatatgcggattagaaaaacaacttaaatgtgaaagtggg
tccgcgtacagcggatcccgggaattctagagggcccgcggttcgaacaaaaactcatctcagaagaggatctga
atatgcataccggttagtaatgagtttggaattaattctgtggaatgtgtgtcagttagggtgtggaaagtcccc
aggctccccaggcaggcagaagtatgcaaagcatgcatctcaattagtcagcaaccaggtgtggaaagtccccag
gctccccagcaggcagaagtatgcaaagcatgcatctcaattagtcagcaaccatagtcccgcccctaactccgc
ccatcccgcccctaactccgcccagttccgcccattctccgccccatggctgactaattttttttatttatgcag
aggccgaggccgcctctgcctctgagctattccagaagtagtgaggaggcttttttggaggcctaggcttttgca
aaaagctcccgggagcttgtatatccattttcggatctgatcagcacgtgttgacaattaatcatcggcatagta
tatcggcatagtataatacgacaaggtgaggaactaaaccatggccaagcctttgtctcaagaagaatccaccct
cattgaaagagcaacggctacaatcaacagcatccccatctctgaagactacagcgtcgccagcgcagctctctc
tagcgacggccgcatcttcactggtgtcaatgtatatcattttactgggggaccttgtgcagaactcgtggtgct
gggcactgctgctgctgcggcagctggcaacctgacttgtatcgtcgcgatcggaaatgagaacaggggcatctt
gagcccctgcggacggtgccgacaggtgcttctcgatctgcatcctgggatcaaagccatagtgaaggacagtga
tggacagccgacggcagttgggattcgtgaattgctgccctctggttatgtgtgggagggctaagcacaattcga
gctcggtacctttaagaccaatgacttacaaggcagctgtagatcttagccactttttaaaagaaaaggggggac
tggaagggctaattcactcccaacgaagacaagatctgctttttgcttgtactgggtctctctggttagaccaga
tctgagcctgggagctctctggctaactagggaacccactgcttaagcctcaataaagcttgccttgagtgcttc
aagtagtgtgtgcccgtctgttgtgtgactctggtaactagagatccctcagacccttttagtcagtgtggaaaa
tctctagcagtagtagttcatgtcatcttattattcagtatttataacttgcaaagaaatgaatatcagagagtg
agaggaacttgtttattgcagcttataatggttacaaataaagcaatagcatcacaaatttcacaaataaagcat
ttttttcactgcattctagttgtggtttgtccaaactcatcaatgtatcttatcatgtctggctctagctatccc
gcccctaactccgcccatcccgcccctaactccgcccagttccgcccattctccgccccatggctgactaatttt
ttttatttatgcagaggccgaggccgcctcggcctctgagctattccagaagtagtgaggaggcttttttggagg
cctagggacgtacccaattcgccctatagtgagtcgtattacgcgcgctcactggccgtcgttttacaacgtcgt
gactgggaaaaccctggcgttacccaacttaatcgccttgcagcacatccccctttcgccagctggcgtaatagc
gaagaggcccgcaccgatcgcccttcccaacagttgcgcagcctgaatggcgaatgggacgcgccctgtagcggc
gcattaagcgcggcgggtgtggtggttacgcgcagcgtgaccgctacacttgccagcgccctagcgcccgctcct
ttcgctttcttcccttcctttctcgccacgttcgccggctttccccgtcaagctctaaatcgggggctcccttta
gggttccgatttagtgctttacggcacctcgaccccaaaaaacttgattagggtgatggttcacgtagtgggcca
tcgccctgatagacggtttttcgccctttgacgttggagtccacgttctttaatagtggactcttgttccaaact
ggaacaacactcaaccctatctcggtctattcttttgatttataagggattttgccgatttcggcctattggtta
aaaaatgagctgatttaacaaaaatttaacgcgaattttaacaaaatattaacgcttacaatttaggtggcactt
ttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgccctta
ttcccttttttgcggcattttgccttcctgtttttgctcacccagaaacgctggtgaaagtaaaagatgctgaag
atcagttgggtgcacgagtgggttacatcgaactggatctcaacagcggtaagatccttgagagttttcgccccg
aagaacgttttccaatgatgagcacttttaaagttctgctatgtggcgcggtattatcccgtattgacgccgggc
aagagcaactcggtcgccgcatacactattctcagaatgacttggttgagtactcaccagtcacagaaaagcatc
ttacggatggcatgacagtaagagaattatgcagtgctgccataaccatgagtgataacactgcggccaacttac
ttctgacaacgatcggaggaccgaaggagctaaccgcttttttgcacaacatgggggatcatgtaactcgccttg
atcgttgggaaccggagctgaatgaagccataccaaacgacgagcgtgacaccacgatgcctgtagcaatggcaa
caacgttgcgcaaactattaactggcgaactacttactctagcttcccggcaacaattaatagactggatggagg
cggataaagttgcaggaccacttctgcgctcggcccttccggctggctggtttattgctgataaatctggagccg
gtgagcgtgggtctcgcggtatcattgcagcactggggccagatggtaagccctcccgtatcgtagttatctaca
cgacggggagtcaggcaactatggatgaacgaaatagacagatcgctgagataggtgcctcactgattaagcatt
ggtaactgtcagaccaagtttactcatatatactttagattgatttaaaacttcatttttaatttaaaaggatct
aggtgaagatcctttttgataatctcatgaccaaaatcccttaacgtgagttttcgttccactgagcgtcagacc
ccgtagaaaagatcaaaggatcttcttgagatcctttttttctgcgcgtaatctgctgcttgcaaacaaaaaaac
caccgctaccagcggtggtttgtttgccggatcaagagctaccaactctttttccgaaggtaactggcttcagca
gagcgcagataccaaatactgttcttctagtgtagccgtagttaggccaccacttcaagaactctgtagcaccgc
ctacatacctcgctctgctaatcctgttaccagtggctgctgccagtggcgataagtcgtgtcttaccgggttgg
actcaagacgatagttaccggataaggcgcagcggtcgggctgaacggggggttcgtgcacacagcccagcttgg
agcgaacgacctacaccgaactgagatacctacagcgtgagctatgagaaagcgccacgcttcccgaagggagaa
aggcggacaggtatccggtaagcggcagggtcggaacaggagagcgcacgagggagcttccagggggaaacgcct
ggtatctttatagtcctgtcgggtttcgccacctctgacttgagcgtcgatttttgtgatgctcgtcaggggggc
ggagcctatggaaaaacgccagcaacgcggcctttttacggttcctggccttttgctggccttttgctcacatgt
tctttcctgcgttatcccctgattctgtggataaccgtattaccgcctttgagtgagctgataccgctcgccgca
gccgaacgaccgagcgcagcgagtcagtgagcgaggaagcggaagagcgcccaatacgcaaaccgcctctccccg
cgcgttggccgattcattaatgcagctggcacgacaggtttcccgactggaaagcgggcagtgagcgcaacgcaa
ttaatgtgagttagctcactcattaggcaccccaggctttacactttatgcttccggctcgtatgttgtgtggaa
ttgtgagcggataacaatttcacacaggaaacagctatgaccatgattacgccaagcgcgcaattaaccctcact
aaagggaacaaaagctggagctgcaagctt

TABLE 33
Nucleotide sequence of pLenti6/V5.

aatgtagtcttatgcaatactcttgtagtcttgcaacatggtaacgatgagttagcaacatgccttacaaggaga
gaaaaagcaccgtgcatgccgattggtggaagtaaggtggtacgatcgtgccttattaggaaggcaacagacggg
tctgacatggattggacgaaccactgaattgccgcattgcagagatattgtatttaagtgcctagctcgatacat
aaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaagggaaacca
gaggagctctctcgacgcaggactcggcttgctgaagcgcgcacggcaagaggcgaggggcggcgactggtgagt
acgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgg
gcaagcagggagctagaacgattcgcagttaatcctggcctgttagaaacatcagaaggctgtagacaaatactg
ggacagctacaaccatcccttcagacaggatcagaagaacttagatcattatataatacagtagcaaccctctat
tgtgtgcatcaaaggatagagataaaagacaccaaggaagctttagacaagatagaggaagagcaaaacaaaagt
aagaccaccgcacagcaagcggccgctgatcttcagacctggaggaggagatatgagggacaattggagaagtga
attatataaatataaagtagtaaaaattgaaccattaggagtagcacccaccaaggcaaagagaagagtggtgca
gagagaaaaaagagcagtgggaataggagctttgttccttgggttcttgggagcagcaggaagcactatgggcgc
agcgtcaatgacgctgacggtacaggccagacaattattgtctggtatagtgcagcagcagaacaatttgctgag
ggctattgaggcgcaacagcatctgttgcaactcacagtctggggcatcaagcagctccaggcaagaatcctggc
tgtggaaagatacctaaaggatcaacagctcctggggatttggggttgctctggaaaactcatttgcaccactgc
tgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatga
acaagaattattggaattagataaatgggcaagtttgtggaattggtttaacataacaaattggctgtggtatat
aaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaagg
aatagaagaagaaggtggagagagagacagagacagatccattcgattagtgaacggatctcgacggtatcgata
agcttgggagttccgcgttacataacttacggtaaatggcccgcctggctgaccgcccaacgacccccgcccatt
gacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacgggg
atttccaagtctccaccccattgacgtcaatgggagtttgttttggcaccaaaatcaacgggactttccaaaatg
tcgtaacaactccgccccattgacgcaaatgggcggtaggcgtgtacggtgggaggtctatataagcagagctcg
tttagtgaaccgtcagatcgcctggagacgccatccacgctgttttgacctccatagaagacaccgactctagag
gatccactagtccagtgtggtggaattctgcagatatccagcacagtggcggccgctcgagtctagagggcccgc
ggttcgaaggtaagcctatccctaaccctctcctcggtctcgattctacgcgtaccggttagtaatgagtttgga
attaattctgtggaatgtgtgtcagttagggtgtggaaagtccccaggctccccaggcaggcagaagtatgcaaa
gcatgcatctcaattagtcagcaaccaggtgtggaaagtccccaggctccccagcaggcagaagtatgcaaagca
tgcatctcaattagtcagcaaccatagtcccgcccctaactccgcccatcccgcccctaactccgcccagttccg
cccattctccgccccatggctgactaattttttttatttatgcagaggccgaggccgcctctgcctctgagctat
tccagaagtagtgaggaggcttttttggaggcctaggcttttgcaaaaagctcccgggagcttgtatatccattt
tcggatctgatcagcacgtgttgacaattaatcatcggcatagtatatcggcatagtataatacgacaaggtgag
gaactaaaccatggccaagcctttgtctcaagaagaatccaccctcattgaaagagcaacggctacaatcaacag
catccccatctctgaagactacagcgtcgccagcgcagctctctctagcgacggccgcatcttcactggtgtcaa
tgtatatcattttactgggggaccttgtgcagaactcgtggtgctgggcactgctgctgctgcggcagctggcaa
cctgacttgtatcgtcgcgatcggaaatgagaacaggggcatcttgagcccctgcggacggtgccgacaggtgct
tctcgatctgcatcctgggatcaaagccatagtgaaggacagtgatggacagccgacggcagttgggattcgtga
attgctgccctctggttatgtgtgggagggctaagcacaattcgagctcggtacctttaagaccaatgacttaca
aggcagctgtagatcttagccactttttaaaagaaaaggggggactggaagggctaattcactcccaacgaagac
aagatctgctttttgcttgtactgggtctctctggttagaccagatctgagcctgggagctctctggctaactag
ggaacccactgcttaagcctcaataaagcttgccttgagtgcttcaagtagtgtgtgcccgtctgttgtgtgact
ctggtaactagagatccctcagacccttttagtcagtgtggaaaatctctagcagtagtagttcatgtcatctta
ttattcagtatttataacttgcaaagaaatgaatatcagagagtgagaggaacttgtttattgcagcttataatg
gttacaaataaagcaatagcatcacaaatttcacaaataaagcatttttttcactgcattctagttgtggtttgt
ccaaactcatcaatgtatcttatcatgtctggctctagctatcccgcccctaactccgcccatcccgcccctaac
tccgcccagttccgcccattctccgccccatggctgactaattttttttatttatgcagaggccgaggccgcctc
ggcctctgagctattccagaagtagtgaggaggcttttttggaggcctagggacgtacccaattcgccctatagt
gagtcgtattacgcgcgctcactggccgtcgttttacaacgtcgtgactgggaaaaccctggcgttacccaactt
aatcgccttgcagcacatccccctttcgccagctggcgtaatagcgaagaggcccgcaccgatcgcccttcccaa
cagttgcgcagcctgaatggcgaatgggacgcgccctgtagcggcgcattaagcgcggcgggtgtggtggttacg
cgcagcgtgaccgctacacttgccagcgccctagcgcccgctcctttcgctttcttcccttcctttctcgccacg
ttcgccggctttccccgtcaagctctaaatcgggggctccctttagggttccgatttagtgctttacggcacctc
gaccccaaaaaacttgattagggtgatggttcacgtagtgggccatcgccctgatagacggtttttcgccctttg
acgttggagtccacgttctttaatagtggactcttgttccaaactggaacaacactcaaccctatctcggtctat
tcttttgatttataagggattttgccgatttcggcctattggttaaaaaatgagctgatttaacaaaaatttaac
gcgaattttaacaaaatattaacgcttacaatttaggtggcacttttcggggaaatgtgcgcggaacccctattt
gtttatttttctaaatacattcaaatatgtatccgctcatgagacaataaccctgataaatgcttcaataatatt
gaaaaaggaagagtatgagtattcaacatttccgtgtcgcccttattcccttttttgcggcattttgccttcctg
tttttgctcacccagaaacgctggtgaaagtaaaagatgctgaagatcagttgggtgcacgagtgggttacatcg
aactggatctcaacagcggtaagatccttgagagttttcgccccgaagaacgttttccaatgatgagcactttta
aagttctgctatgtggcgcggtattatcccgtattgacgccgggcaagagcaactcggtcgccgcatacactatt
ctcagaatgacttggttgagtactcaccagtcacagaaaagcatcttacggatggcatgacagtaagagaattat
gcagtgctgccataaccatgagtgataacactgcggccaacttacttctgacaacgatcggaggaccgaaggagc
taaccgcttttttgcacaacatgggggatcatgtaactcgccttgatcgttgggaaccggagctgaatgaagcca
taccaaacgacgagcgtgacaccacgatgcctgtagcaatggcaacaacgttgcgcaaactattaactggcgaac
tacttactctagcttcccggcaacaattaatagactggatggaggcggataaagttgcaggaccacttctgcgct
cggcccttccggctggctggtttattgctgataaatctggagccggtgagcgtgggtctcgcggtatcattgcag
cactggggccagatggtaagccctcccgtatcgtagttatctacacgacggggagtcaggcaactatggatgaac
gaaatagacagatcgctgagataggtgcctcactgattaagcattggtaactgtcagaccaagtttactcatata
tactttagattgatttaaaacttcatttttaatttaaaaggatctaggtgaagatcctttttgataatctcatga
ccaaaatcccttaacgtgagttttcgttccactgagcgtcagaccccgtagaaaagatcaaaggatcttcttgag
atcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgttcttctag
tgtagccgtagttaggccaccacttcaagaactctgtagcaccgcctacatacctcgctctgctaatcctgttac
cagtggctgctgccagtggcgataagtcgtgtcttaccgggttggactcaagacgatagttaccggataaggcgc
agcggtcgggctgaacggggggttcgtgcacacagcccagcttggagcgaacgacctacaccgaactgagatacc
tacagcgtgagctatgagaaagcgccacgcttcccgaagggagaaaggcggacaggtatccggtaagcggcaggg
tcggaacaggagagcgcacgagggagcttccagggggaaacgcctggtatctttatagtcctgtcgggtttcgcc
acctctgacttgagcgtcgatttttgtgatgctcgtcaggggggcggagcctatggaaaaacgccagcaacgcgg
cctttttacggttcctggccttttgctggccttttgctcacatgttctttcctgcgttatcccctgattctgtgg
ataaccgtattaccgcctttgagtgagctgataccgctcgccgcagccgaacgaccgagcgcagcgagtcagtga
gcgaggaagcggaagagcgcccaatacgcaaaccgcctctccccgcgcgttggccgattcattaatgcagctggc
acgacaggtttcccgactggaaagcgggcagtgagcgcaacgcaattaatgtgagttagctcactcattaggcac
cccaggctttacactttatgcttccggctcgtatgttgtgtggaattgtgagcggataacaatttcacacaggaa
acagctatgaccatgattacgccaagcgcgcaattaaccctcactaaagggaacaaaagctggagctgcaagctt

TABLE 34
Nucleotide sequence of pLenti3/V5-TREx.

aatgtagtcttatgcaatactcttgtagtcttgcaacatggtaacgatgagttagcaacatgccttacaaggaga
gaaaaagcaccgtgcatgccgattggtggaagtaaggtggtacgatcgtgccttattaggaaggcaacagacggg
tctgacatggattggacgaaccactgaattgccgcattgcagagatattgtatttaagtgcctagctcgatacat
aaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaagggaaacca
gaggagctctctcgacgcaggactcggcttgctgaagcgcgcacggcaagaggcgaggggcggcgactggtgagt
acgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgg
gcaagcagggagctagaacgattcgcagttaatcctggcctgttagaaacatcagaaggctgtagacaaatactg
ggacagctacaaccatcccttcagacaggatcagaagaacttagatcattatataatacagtagcaaccctctat
tgtgtgcatcaaaggatagagataaaagacaccaaggaagctttagacaagatagaggaagagcaaaacaaaagt
aagaccaccgcacagcaagcggccgctgatcttcagacctggaggaggagatatgagggacaattggagaagtga
attatataaatataaagtagtaaaaattgaaccattaggagtagcacccaccaaggcaaagagaagagtggtgca
gagagaaaaaagagcagtgggaataggagctttgttccttgggttcttgggagcagcaggaagcactatgggcgc
agcgtcaatgacgctgacggtacaggccagacaattattgtctggtatagtgcagcagcagaacaatttgctgag
ggctattgaggcgcaacagcatctgttgcaactcacagtctggggcatcaagcagctccaggcaagaatcctggc
tgtggaaagatacctaaaggatcaacagctcctggggatttggggttgctctggaaaactcatttgcaccactgc
tgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatga
acaagaattattggaattagataaatgggcaagtttgtggaattggtttaacataacaaattggctgtggtatat
aaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaagg
aatagaagaagaaggtggagagagagacagagacagatccattcgattagtgaacggatctcgacggtatcgata
agcttgggagttccgcgttacataacttacggtaaatggcccgcctggctgaccgcccaacgacccccgcccatt
gacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacgggg
atttccaagtctccaccccattgacgtcaatgggagtttgttttggcaccaaaatcaacgggactttccaaaatg
tcgtaacaactccgccccattgacgcaaatgggcggtaggcgtgtacggtgggaggtctatataagcagagctct
ccctatcagtgatagagatctccctatcagtgatagagatcgtcgacgagctcgtttagtgaaccgtcagatcgc
ctggagacgccatccacgctgttttgacctccatagaagacaccgggaccgatccagcctccggactctagagga
tccctaccggtgatatcctcgagtctagagggcccgcggttcgaaggtaagcctatccctaaccctctcctcggt
ctcgattctacgcgtaccggttagtaatgagtttggaattaattctgtggaatgtgtgtcagttagggtgtggaa
agtccccaggctccccaggcaggcagaagtatgcaaagcatgcatctcaattagtcagcaaccaggtgtggaaag
tccccaggctccccagcaggcagaagtatgcaaagcatgcatctcaattagtcagcaaccatagtcccgccccta
actccgcccatcccgcccctaactccgcccagttccgcccattctccgccccatggctgactaattttttttatt
tatgcagaggccgaggccgcctctgcctctgagctattccagaagtagtgaggaggcttttttggaggcctaggc
ttttgcaaaaagctccccctgttgacaattaatcatcggcatagtatatcggcatagtataatacgacaaggtga
ggaactaaaccatggcctcaattgaacaagatggattgcacgcaggttctccggccgcttgggtggagaggctat
tcggctatgactgggcacaacagacaatcggctgctctgatgccgccgtgttccggctgtcagcgcaggggcgcc
cggttctttttgtcaagaccgacctgtccggtgccctgaatgaactgcaggacgaggcagcgcggctatcgtggc
tggccacgacgggcgttccttgcgcagctgtgctcgacgttgtcactgaagcgggaagggactggctgctattgg
gcgaagtgccggggcaggatctcctgtcatctcaccttgctcctgccgagaaagtatccatcatggctgatgcaa
tgcggcggctgcatacgcttgatccggctacctgcccattcgaccaccaagcgaaacatcgcatcgagcgagcac
gtactcggatggaagccggtcttgtcgatcaggatgatctggacgaagagcatcaggggctcgcgccagccgaac
tgttcgccaggctcaaggcgcgcatgcccgacggcgaggatctcgtcgtgacccatggcgatgcctgcttgccga
atatcatggtggaaaatggccgcttttctggattcatcgactgtggccggctgggtgtggcggaccgctatcagg
acatagcgttggctacccgtgatattgctgaagagcttggcggcgaatgggctgaccgcttcctcgtgctttacg
gtatcgccgctcccgattcgcagcgcatcgccttctatcgccttcttgacgagttcttctgagcgggactctggg
gttcgaaatgaccgaccaagcgacgcccaacctgccatcacgagtttaaactggtacctttaagaccaatgactt
acaaggcagctgtagatcttagccactttttaaaagaaaaggggggactggaagggctaattcactcccaacgaa
gacaagatctgctttttgcttgtactgggtctctctggttagaccagatctgagcctgggagctctctggctaac
tagggaacccactgcttaagcctcaataaagcttgccttgagtgcttcaagtagtgtgtgcccgtctgttgtgtg
actctggtaactagagatccctcagacccttttagtcagtgtggaaaatctctagcagtagtagttcatgtcatc
ttattattcagtatttataacttgcaaagaaatgaatatcagagagtgagaggaacttgtttattgcagcttata
atggttacaaataaagcaatagcatcacaaatttcacaaataaagcatttttttcactgcattctagttgtggtt
tgtccaaactcatcaatgtatcttatcatgtctggctctagctatcccgcccctaactccgcccatcccgcccct
aactccgcccagttccgcccattctccgccccatggctgactaattttttttatttatgcagaggccgaggccgc
ctcggcctctgagctattccagaagtagtgaggaggcttttttggaggcctagggacgtacccaattcgccctat
agtgagtcgtattacgcgcgctcactggccgtcgttttacaacgtcgtgactgggaaaaccctggcgttacccaa
cttaatcgccttgcagcacatccccctttcgccagctggcgtaatagcgaagaggcccgcaccgatcgcccttcc
caacagttgcgcagcctgaatggcgaatgggacgcgccctgtagcggcgcattaagcgcggcgggtgtggtggtt
acgcgcagcgtgaccgctacacttgccagcgccctagcgcccgctcctttcgctttcttcccttcctttctcgcc
acgttcgccggctttccccgtcaagctctaaatcgggggctccctttagggttccgatttagtgctttacggcac
ctcgaccccaaaaaacttgattagggtgatggttcacgtagtgggccatcgccctgatagacggtttttcgccct
ttgacgttggagtccacgttctttaatagtggactcttgttccaaactggaacaacactcaaccctatctcggtc
tattcttttgatttataagggattttgccgatttcggcctattggttaaaaaatgagctgatttaacaaaaattt
aacgcgaattttaacaaaatattaacgcttacaatttaggtggcacttttcggggaaatgtgcgcggaaccccta
tttgtttatttttctaaatacattcaaatatgtatccgctcatgagacaataaccctgataaatgcttcaataat
attgaaaaaggaagagtatgagtattcaacatttccgtgtcgcccttattcccttttttgcggcattttgccttc
ctgtttttgctcacccagaaacgctggtgaaagtaaaagatgctgaagatcagttgggtgcacgagtgggttaca
tcgaactggatctcaacagcggtaagatccttgagagttttcgccccgaagaacgttttccaatgatgagcactt
ttaaagttctgctatgtggcgcggtattatcccgtattgacgccgggcaagagcaactcggtcgccgcatacact
attctcagaatgacttggttgagtactcaccagtcacagaaaagcatcttacggatggcatgacagtaagagaat
tatgcagtgctgccataaccatgagtgataacactgcggccaacttacttctgacaacgatcggaggaccgaagg
agctaaccgcttttttgcacaacatgggggatcatgtaactcgccttgatcgttgggaaccggagctgaatgaag
ccataccaaacgacgagcgtgacaccacgatgcctgtagcaatggcaacaacgttgcgcaaactattaactggcg
aactacttactctagcttcccggcaacaattaatagactggatggaggcggataaagttgcaggaccacttctgc
gctcggcccttccggctggctggtttattgctgataaatctggagccggtgagcgtgggtctcgcggtatcattg
cagcactggggccagatggtaagccctcccgtatcgtagttatctacacgacggggagtcaggcaactatggatg
aacgaaatagacagatcgctgagataggtgcctcactgattaagcattggtaactgtcagaccaagtttactcat
atatactttagattgatttaaaacttcatttttaatttaaaaggatctaggtgaagatcctttttgataatctca
tgaccaaaatcccttaacgtgagttttcgttccactgagcgtcagaccccgtagaaaagatcaaaggatcttctt
gagatcctttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatactgttcttc
tagtgtagccgtagttaggccaccacttcaagaactctgtagcaccgcctacatacctcgctctgctaatcctgt
taccagtggctgctgccagtggcgataagtcgtgtcttaccgggttggactcaagacgatagttaccggataagg
cgcagcggtcgggctgaacggggggttcgtgcacacagcccagcttggagcgaacgacctacaccgaactgagat
acctacagcgtgagctatgagaaagcgccacgcttcccgaagggagaaaggcggacaggtatccggtaagcggca
gggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacg
cggcctttttacggttcctggccttttgctggccttttgctcacatgttctttcctgcgttatcccctgattctg
tggataaccgtattaccgcctttgagtgagctgataccgctcgccgcagccgaacgaccgagcgcagcgagtcag
tgagcgaggaagcggaagagcgcccaatacgcaaaccgcctctccccgcgcgttggccgattcattaatgcagct
ggcacgacaggtttcccgactggaaagcgggcagtgagcgcaacgcaattaatgtgagttagctcactcattagg
caccccaggctttacactttatgcttccggctcgtatgttgtgtggaattgtgagcggataacaatttcacacag
gaaacagctatgaccatgattacgccaagcgcgcaattaaccctcactaaagggaacaaaagctggagctgcaag
ctt

TABLE 35

Nucleotide sequence of a nucleic acid fragment containing the tetracycline
repressor coding sequence.

agcttggtacccggggatcctctagggcctctgagctattccagaagtagtgaagaggcttttttggaggcctag
gcttttgcaaaaagctccggatcgatcctgagaacttcagggtgagtttggggacccttgattgttctttctttt
tcgctattgtaaaattcatgttatatggagggggcaaagttttcagggtgttgtttagaatgggaagatgtccct
tgtatcaccatggaccctcatgataattttgtttctttcactttctactctgttgacaaccattgtctcctctta
ttttcttttcattttctgtaactttttcgttaaactttagcttgcatttgtaacgaatttttaaattcacttttg
tttatttgtcagattgtaagtactttctctaatcacttttttttcaaggcaatcagggtatattatattgtactt
cagcacagttttagagaacaattgttataattaaatgataaggtagaatatttctgcatataaattctggctggc
gtggaaatattcttattggtagaaacaactacatcctggtcatcatcctgcctttctctttatggttacaatgat
atacactgtttgagatgaggataaaatactctgagtccaaaccgggcccctctgctaaccatgttcatgccttct
tctttttcctacagctcctgggcaacgtgctggttattgtgctgtctcatcattttggcaaagaattgtaatacg
actcactatagggcgaattgatatgtctagattagataaaagtaaagtgattaacagcgcattagagctgcttaa
tgaggtcggaatcgaaggtttaacaacccgtaaactcgcccagaagctaggtgtagagcagcctacattgtattg
gcatgtaaaaaataagcgggctttgctcgacgccttagccattgagatgttagataggcaccatactcacttttg
ccctttagaaggggaaagctggcaagattttttacgtaataacgctaaaagttttagatgtgctttactaagtca
tcgcgatggagcaaaagtacatttaggtacacggcctacagaaaaacagtatgaaactctcgaaaatcaattagc
ctttttatgccaacaaggtttttcactagagaatgcattatatgcactcagcgctgtggggcattttactttagg
ttgcgtattggaagatcaagagcatcaagtcgctaaagaagaaagggaaacacctactactgatagtatgccgcc
attattacgacaagctatcgaattatttgatcaccaaggtgcagagccagccttcttattcggccttgaattgat
catatgcggattagaaaaacaacttaaatgtgaaagtgggtccgcgtacagcggatcccgggaattctagagggc
ccgcggttcgaacaaaaactcatctcagaagaggatctgaatatgcata

TABLE 36
Nucleotide sequence of pRRL6/V5 also referred to as pLenti6/V5.

| | |
|---|---|
| 1 | aatgtagtct tatgcaatac tcttgtagtc ttgcaacatg gtaacgatga gttagcaaca |
| 61 | tgccttacaa ggagagaaaa agcaccgtgc atgccgattg gtggaagtaa ggtggtacga |
| 121 | tcgtgcctta ttaggaaggc aacagacggg tctgacatgg attggacgaa ccactgaatt |
| 181 | gccgcattgc agagatattg tatttaagtg cctagctcga tacaataaac gggtctctct |
| 241 | ggttagacca gatctgagcc tgggagctct ctggctaact agggaaccca ctgcttaagc |
| 301 | ctcaataaag cttgccttga gtgcttcaag tagtgtgtgc ccgtctgttg tgtgactctg |
| 361 | gtaactagag atccctcaga cccttttagt cagtgtggaa aatctctagc agtggcgccc |
| 421 | gaacagggac ctgaaagcga aagggaaacc agagctctct cgacgcagga ctcggcttgc |
| 481 | tgaagcgcgc acggcaagag gcgaggggcg gcgactggtg agtacgccaa aaattttgac |
| 541 | tagcggaggc tagaaggaga gagatgggtg cgagagcgtc agtattaagc gggggagaat |
| 601 | tagatcgcga tgggaaaaaa ttcggttaag gccaggggga aagaaaaaat ataaattaaa |
| 661 | acatatagta tgggcaagca gggagctaga acgattcgca gttaatcctg gcctgttaga |
| 721 | aacatcagaa ggctgtagac aaatactggg acagctacaa ccatcccttc agacaggatc |
| 781 | agaagaactt agatcattat ataatacagt agcaaccctc tattgtgtgc atcaaaggat |
| 841 | agagataaaa gacaccaagg aagctttaga caagatagag gaagagcaaa acaaaagtaa |
| 901 | gaccaccgca cagcaagcgg ccgctgatct tcagacctgg aggaggagat atgagggaca |
| 961 | attggagaag tgaattatat aaatataaag tagtaaaaat tgaaccatta ggagtagcac |
| 1021 | ccaccaaggc aaagagaaga gtggtgcaga gagaaaaaag agcagtggga ataggagctt |
| 1081 | tgttccttgg gttcttggga gcagcaggaa gcactatggg cgcagcctca atgacgctga |
| 1141 | cggtacaggc cagacaatta ttgtctggta tagtgcagca gcagaacaat ttgctgaggg |
| 1201 | ctattgaggc gcaacagcat ctgttgcaac tcacagtctg gggcatcaag cagctccagg |
| 1261 | caagaatcct ggctgtggaa agatacctaa aggatcaaca gctcctgggg atttggggtt |
| 1321 | gctctggaaa actcatttgc accactgctg tgccttggaa tgctagttgg agtaataaat |
| 1381 | ctctggaaca gattggaatc acacgacctg gatggagtgg gacagagaaa ttaacaatta |
| 1441 | cacaagctta atacactcct taattgaaga atcgcaaaac cagcaagaaa agaatgaaca |
| 1501 | agaattattg gaattagata aatgggcaag tttgtggaat tggtttaaca taacaaattg |
| 1561 | gctgtggtat ataaaattat tcataatgat agtaggaggc ttggtaggtt taagaatagt |
| 1621 | ttttgctgta ctttctatag tgaatagagt taggcaggga tattcaccat tatcgtttca |
| 1681 | gacccacctc ccaaccccga ggggacccga caggcccgaa ggaatagaag aagaaggtgg |
| 1741 | agagagagac agagacagat ccattcgatt agtgaacgga tctcgacggt atcgataagc |
| 1801 | ttgggagttc cgcgttacat aacttacggt aaatggcccg cctggctgac cgcccaacga |
| 1861 | cccccgccca ttgacgtcaa taatgacgta tgttcccata gtaacgccaa tagggacttt |
| 1921 | ccattgacgt caatgggtgg agtatttacg gtaaactgcc cacttggcag tacatcaagt |
| 1981 | gtatcatatg ccaagtacgc cccctattga cgtcaatgac ggtaaatggc ccgcctggca |
| 2041 | ttatgcccag tacatgacct tatgggactt tcctacttgg cagtacatct acgtattagt |
| 2101 | catcgctatt accatggtga tgcggttttg gcagtacatc aatgggcgtg gatagcggtt |
| 2161 | tgactcacgg ggatttccaa gtctccaccc cattgacgtc aatgggagtt tgttttggca |
| 2221 | ccaaaatcaa cgggactttc caaaatgtcg taacaactcc gccccattga cgcaaatggg |
| 2281 | cggtaggcgt gtacggtggg aggtctatat aagcagagct cgtttagtga accgtcagat |
| 2341 | cgcctggaga cgccatccac gctgttttga cctccataga agacaccgac tctagaggat |
| 2401 | ccactagtcc agtgtggtgg aattctgcag atatccagca cagtggcggc cgctcgagtc |
| 2461 | tagagggccc gcggttcgaa ggtaagccta tccctaaccc tctcctcggt ctcgattcta |
| 2521 | cgcgtaccgg ttagtaatga gtttggcctg ctgccggctc tgcggcctct tccgcgtctt |
| 2581 | cgccttcgcc ctcagacgag tcggatctcc ctttgggccg cctccccgcc tggaattaat |
| 2641 | tctgtggaat gtgtgtcagt tagggtgtgg aaagtcccca ggctccccag gcaggcagaa |
| 2701 | gtatgcaaag catgcatctc aattagtcag caaccaggtg tggaaagtcc ccaggctccc |
| 2761 | cagcaggcag aagtatgcaa agcatgcatc tcaattagtc agcaaccata gtcccgcccc |
| 2821 | taactccgcc catcccgccc ctaactccgc ccagttccgc ccattctccg ccccatggct |
| 2881 | gactaatttt ttttatttat gcagaggccg aggccgcctc tgcctctgag ctattccaga |
| 2941 | agtagtgagg aggctttttt ggaggcctag gcttttgcaa aaagctcccg ggagcttgta |
| 3001 | tatccatttt cggatctgat cagcacgtgt tgacaattaa tcatcggcat agtatatcgg |
| 3061 | catagtataa tacgacaagg tgaggaacta aaccatggcc aagcctttgt ctcaagaaga |
| 3121 | atccaccctc attgaaagag caacggctac aatcaacagc atccccatct ctgaagacta |
| 3181 | cagcgtcgcc agcgcagctc tctctagcga cggccgcatc ttcactggtg tcaatgtata |
| 3241 | tcattttact gggggacctt gtgcagaact cgtggtgctg ggcactgctg ctgctgcggc |
| 3301 | agctggcaac ctgacttgta tcgtcgcgat cggaaatgag aacaggggca tcttgagccc |
| 3361 | ctgcggacgg tgccgacagg tgcttctcga tctgcatcct gggatcaaag ccatagtgaa |
| 3421 | ggacagtgat ggacagccga cggcagttgg gattcgtgaa ttgctgccct ctggttatgt |
| 3481 | gtgggagggc taagcacaat tcgagctcgg tacctttaag accaatgact tacaaggcag |
| 3541 | ctgtagatct tagccacttt ttaaaagaaa aggggggact ggaagggcta attcactccc |
| 3601 | aacgaagaca agatctgctt tttgcttgta ctgggtctct ctggttagac cagatctgag |
| 3661 | cctgggagct ctctggctaa ctagggaacc cactgcttaa gcctcaataa agcttgcctt |
| 3721 | gagtycttca agtagtgtgt gcccgtctgt tgtgtgactc tggtaactag agatccctca |
| 3781 | gaccctttta gtcagtgtgg aaaatctcta gcagtagtag ttcatgtcat cttattattc |
| 3841 | agtatttata acttgcaaag aaatgaatat cagagagtga gaggaacttg tttattgcag |
| 3901 | cttataatgg ttacaaataa agcaatagca tcacaaattt cacaaataaa gcattttttt |
| 3961 | cactgcattc tagttgtggt ttgtccaaac tcatcaatgt atcttatcat gtctggctct |
| 4021 | agctatcccg cccctaactc cgcccagttc cgcccattct ccgccccatg gctgactaat |
| 4081 | tttttttatt tatgcagagg ccgaggccgc ctcggcctct gagctattcc agaagtagtg |
| 4141 | aggaggcttt tttggaggcc taggcttttg cgtcgagacg tacccaattc gccctatagt |
| 4201 | gagtcgtatt acgcgcgctc actggccgtc gttttacaac gtcgtgactg ggaaaaccct |
| 4261 | ggcgttaccc aacttaatcg ccttgcagca catccccctt tcgccagctg gcgtaatagc |
| 4321 | gaagaggccc gcaccgatcg cccttcccaa cagttgcgca gcctgaatgg cgaatggcgc |
| 4381 | gacgcgccct gtagcggcgc attaagcgcg gcgggtgtgg tggttacgcg cagcgtgacc |
| 4441 | gctacacttg ccagcgccct agcgcccgct cctttcgctt tcttcccttc ctttctcgcc |
| 4501 | acgttcgccg gctttccccg tcaagctcta aatcgggggc tccctttagg gttccgattt |
| 4561 | agtgctttac ggcacctcga ccccaaaaaa cttgattagg gtgatggttc acgtagtggg |
| 4621 | ccatcgccct gatagacggt ttttcgccct ttgacgttgg agtccacgtt ctttaatagt |
| 4681 | ggactcttgt tccaaactgg aacaacactc aaccctatct cggtctattc ttttgattta |
| 4741 | taagggattt tgccgatttc ggcctattgg ttaaaaaatg agctgattta acaaaaattt |
| 4801 | aacgcgaatt ttaacaaaat attaacgttt acaatttccc aggtggcact tttcggggaa |
| 4861 | atgtgcgcgg aacccctatt tgtttatttt tctaaataca ttcaaatatg tatccgctca |
| 4921 | tgagacaata accctgataa atgcttcaat aatattgaaa aaggaagagt atgagtattc |
| 4981 | aacatttccg tgtcgccctt attccctttt ttgcggcatt ttgccttcct gtttttgctc |
| 5041 | acccagaaac gctggtgaaa gtaaaagatg ctgaagatca gttgggtgca cgagtgggtt |
| 5101 | acatcgaact ggatctcaac agcggtaaga tccttgagag ttttcgcccc gaagaacgtt |
| 5161 | ttccaatgat gagcactttt aaagttctgc tatgtggcgc ggtattatcc cgtattgacg |
| 5221 | ccgggcaaga gcaactcggt cgccgcatac actattctca gaatgacttg gttgagtact |
| 5281 | caccagtcac agaaaagcat cttacggatg gcatgacagt aagagaatta tgcagtgctg |
| 5341 | ccataaccat gagtgataac actgcggcca acttacttct gacaacgatc ggaggaccga |
| 5401 | aggagctaac cgcttttttg cacaacatgg gggatcatgt aactcgcctt gatcgttggg |
| 5461 | aaccggagct gaatgaagcc ataccaaacg acgagcgtga caccacgatg cctgtagcaa |
| 5521 | tggcaacaac gttgcgcaaa ctattaactg gcgaactact tactctagct tcccggcaac |
| 5581 | aattaataga ctggatggag gcggataaag ttgcaggacc acttctgcgc tcggcccttc |
| 5641 | cggctggctg gtttattgct gataaatctg gagccggtga gcgtgggtct cgcggtatca |
| 5701 | ttgcagcact ggggccagat ggtaagccct cccgtatcgt agttatctac acgacgggga |
| 5761 | gtcaggcaac tatggatgaa cgaaatagac agatcgctga gataggtgcc tcactgatta |
| 5821 | agcattggta actgtcagac caagtttact catatatact ttagattgat ttaaaacttc |
| 5881 | atttttaatt taaaaggatc taggtgaaga tcctttttga taatctcatg accaaaatcc |
| 5941 | cttaacgtga gttttcgttc cactgagcgt cagaccccgt agaaaagatc aaaggatctt |
| 6001 | cttgagatcc tttttttctg cgcgtaatct gctgcttgca aacaaaaaaa ccaccgctac |
| 6061 | cagcggtggt ttgtttgccg gatcaagagc taccaactct ttttccgaag gtaactggct |
| 6121 | tcagcagagc gcagatacca aatactgtcc ttctagtgta gccgtagtta ggccaccact |
| 6181 | tcaagaactc tgtagcaccg cctacatacc tcgctctgct aatcctgtta ccagtggctg |
| 6241 | ctgccagtgg cgataagtcg tgtcttaccg ggttggactc aagacgatag ttaccggata |
| 6301 | aggcgcagcg gtcgggctga acggggggtt cgtgcacaca gcccagcttg gagcgaacga |
| 6361 | cctacaccga actgagatac ctacagcgtg agctatgaga aagcgccacg cttcccgaag |
| 6421 | ggagaaaggc ggacaggtat ccggtaagcg gcagggtcgg aacaggagag cgcacgaggg |
| 6481 | agcttccagg gggaaacgcc tggtatcttt atagtcctgt cgggtttcgc cacctctgac |
| 6541 | ttgagcgtcg atttttgtga tgctcgtcag gggggcggag cctatggaaa aacgccagca |
| 6601 | acgcggcctt tttacggttc ctggcctttt gctggccttt tgctcacatg ttctttcctg |
| 6661 | cgttatcccc tgattctgtg gataaccgta ttaccgcctt tgagtgagct gataccgctc |
| 6721 | gccgcagccg aacgaccgag cgcagcgagt cagtgagcga ggaagcggaa gagcgcccaa |
| 6781 | tacgcaaacc gcctctcccc gcgcgttggc cgattcatta atgcagctgg cacgacaggt |
| 6841 | ttcccgactg gaaagcgggc agtgagcgca acgcaattaa tgtgagttag ctcactcatt |
| 6901 | aggcacccca ggctttacac tttatgcttc cggctcgtat gttgtgtgga attgtgagcg |
| 6961 | gataacaatt tcacacagga aacagctatg accatgatta cgccaagcgc gcaattaacc |
| 7021 | ctcactaaag ggaacaaaag ctggagctgc aagctt |

What is claimed is:

1. A nucleic acid molecule comprising all or a portion of at least one viral genome and further comprising at least two recombination sites that do not substantially recombine with each other, wherein at least one recombination site is capable of undergoing recombination with a compatible recombination site in the presence of at least one protein active in lambda recombination and wherein the nucleic acid molecule replicates in prokaryotic and eukaryotic cells.

2. A nucleic acid molecule according to claim 1, wherein the nucleic acid molecule comprises all or a portion of at least one viral genome selected from the group consisting of an adenovirus genome and an adeno-associate virus genome.

3. A nucleic acid molecule according to claim 1, wherein the nucleic acid molecule comprises all or a portion of at least one retroviral genome.

4. A nucleic acid molecule according to claim 3, wherein the retroviral genome is a lentiviral genome.

5. A nucleic acid molecule according to claim 1, wherein the nucleic acid molecule comprises all or a portion of at least one viral genome selected from the group consisting of a herpesvirus genome and a pox virus genome.

6. A nucleic acid molecule according to claim 1, wherein the nucleic acid molecule comprises all or a portion of at least one RNA virus genome.

7. A nucleic acid molecule according to claim 6, wherein the RNA virus is selected from a group consisting of a flavivirus genome, a togavirus genome, and an alphavirus genome.

8. A nucleic acid molecule according to claim 1, wherein the nucleic acid molecule is a plasmid or a bacmid comprising a prokaryotic origin of replication and a selectable marker.

9. A nucleic acid molecule according to claim 1, further comprising one or more features selected from the group consisting of a promoter, a viral terminal repeat, a splice site, a packaging signal, a nucleic acid sequence responsive to one or more viral proteins, a recognition site, a recombination site, a sequences encoding one or more marker proteins or polypeptides, a sequence encoding one or more epitopes recognizable by an antibody, an origin of replication, an intervening sequence, an internal ribosome entry sequences, and a polyadenylation signal.

10. A nucleic acid molecule according to claim 1, further comprising a nucleotide sequence of interest between the two recombination sites.

11. A nucleic acid molecule according to claim 10, wherein the sequence of interest comprises one or more sequences selected from the group consisting a sequence encoding one or more polypeptides, a sequence encoding one or more tRNA sequences, a sequence encoding one or more ribozyme sequences, one or more promoter sequences, one or more enhancer sequences, and one or more repressor sequences.

12. A nucleic acid molecule comprising all or a portion of a baculoviral genome and further comprising one or more recombination sites, wherein at least one recombination site is capable of undergoing recombination with a compatible recombination site in the presence of at least one protein active in lambda recombination.

13. A nucleic acid molecule according to claim 12, wherein the molecule comprises two recombination sites that do not substantially recombine with each other.

14. A nucleic acid molecule according to claim 13, wherein the sequence of interest comprises one or more sequences selected from the group consisting a sequence encoding one or more polypeptides, a sequence encoding one or more tRNA sequences, a sequence encoding one or more ribozyme sequences, one or more promoter sequences, one or more enhancer sequences, and one or more repressor sequences.

15. A composition comprising the nucleic acid molecule of claim 1.

16. A composition comprising the nucleic acid molecule of claim 12.

17. A method of constructing a recombinant virus, comprising:
(a) providing a first nucleic acid molecule comprising all or a portion of at least one viral genome and at least a first and a second recombination site that do not substantially recombine with each other;
(b) contacting the first nucleic acid molecule with a second nucleic acid molecule comprising a sequence of interest flanked by at least a third and a fourth recombination site under conditions such that recombination occurs between the first and third recombination site and between the second and fourth recombination site; and
(c) introducing the nucleic acid molecule of step (b) into a cell that packages the nucleic acid molecule of step (b).

18. A method according to claim 17, wherein the first nucleic acid molecule comprises all or a portion of at least one viral genome selected from the group consisting of an adenovirus genome and an adeno-associate virus genome.

19. A method according to claim 17, wherein the first nucleic acid molecule comprises all or a portion of at least one retroviral genome.

20. A method according to claim 19, wherein the retroviral genome is a lentiviral genome.

21. A method according to claim 17, wherein the first nucleic acid molecule comprises all or a portion of at least one viral genome selected from the group consisting of a herpesvirus genome and a pox virus genome.

22. A method according to claim 17, wherein the first nucleic acid molecule comprises all or a portion of at least one RNA virus genome.

23. A method according to claim 22, wherein the RNA virus is selected from a group consisting of a flavivirus genome, a togavirus genome, and an alphavirus genome.

24. A method according to claim 17, wherein the first nucleic acid molecule is a plasmid or a bacmid comprising an origin of replication and a selectable marker.

25. A method according to claim 17, wherein the portion of the second nucleic acid between the recombination site comprises a nucleotide sequence of interest.

26. A method according to claim 25, wherein the sequence of interest comprises one or more sequences selected from a group consisting a sequence encoding one or more polypeptides, a sequence encoding one or more tRNA sequences, a sequence encoding one or more ribozyme sequences, one or more promoter sequences, one or more enhancer sequences, and one or more repressor sequences.

27. A method according to claim 17, further comprising digesting the first nucleic acid molecule with a restriction enzyme that cleaves the first nucleic acid at a site between the recombination sites.

28. A recombinant virus produced by the method of claim 17.

29. A composition comprising the recombinant virus of claim 28.

30. A method of producing a fusion polypeptide, comprising:
providing a host cell comprising a first nucleic acid sequence encoding the fusion polypeptide, wherein the sequence comprises at least a first coding region and a second coding region separated by a sequence comprising a stop codon;
expressing in the cell a second nucleic acid sequence comprising one or more suppressor tRNAs that suppress the stop codon; and
incubating the host cell under conditions sufficient to express the fusion polypeptide comprising the first coding region and the second coding region, wherein at least one of the first and/or second nucleic acid sequences is present on a nucleic acid molecule comprising all or a portion of at least one viral genome.

31. A method according to claim 30, further comprising introducing nucleic acid molecules comprising the first and the second nucleic acid sequences into the host cell.

32. A method according to claim 30, wherein the nucleic acid molecule comprising the second nucleic sequence comprises all or a portion of an adenoviral genome.

33. A method according to claim 30, wherein the first coding region and stop codon are flanked by recombination sites that do not substantially recombine with each other.

34. A method of expressing a polypeptide, comprising:
contacting a cell with a nucleic acid molecule comprising a sequence encoding the polypeptide operably linked to a promoter and a repressor sequence, wherein the nucleic acid molecule comprises all or a portion of a viral genome;
contacting the cell with a nucleic acid molecule encoding a protein that binds to the repressor sequence; and
incubating the cell under conditions sufficient to express the polypeptide.

35. A method according to claim 34, wherein the viral genome is a lentivirus genome.

36. The method according to claim 34, wherein the viral genome is an HIV-1 genome.

37. The method according to claim 34, wherein the repressor sequence is the tetracycline operator sequence and the protein is the tetracycline repressor protein.

38. The method according to claim 37, wherein conditions sufficient to express the polypeptide comprise incubating the cell in the presence of tetracycline.

39. A method of expressing a polypeptide, comprising:
contacting a cell with a nucleic acid molecule comprising a sequence encoding the polypeptide operably linked to a promoter and a repressor sequence, wherein the nucleic acid molecule comprises all or a portion of a viral genome and wherein the cell expresses a protein that binds to the repressor sequence; and
incubating the cell under conditions sufficient to express the polypeptide.

40. The method according to claim 39, wherein the viral genome is a lentiviral genome.

41. The method according to claim 39, wherein the viral genome is an HIV-1 genome.

42. The method according to claim 39, wherein the repressor sequence is the tetracycline operator sequence and the protein is the tetracycline repressor protein.

43. The method according to claim 42, wherein conditions sufficient to express the polypeptide comprise incubating the cell in the presence of tetracycline.
